(19) United States
(12) Patent Application Publication
(10) Pub. No.: US 2005/0144669 A1
(43) Pub. Date: Jun. 30, 2005

(54) MicroRNAs in plants

(75) Inventors: Brenda J. Reinhart, Palo Alto, CA (US); Earl G. Weinstein, Cambridge, MA (US); Matthew W. Rhoades, Somerville, MA (US); Bonnie Bartel, Houston, TX (US); David P. Bartel, Brookline, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignees: Whitehead Institute for Biomedical Research, Cambridge, MA (US); William M. Rice University, Houston, TX (US)

(21) Appl. No.: 10/884,374

(22) Filed: Jul. 1, 2004

Related U.S. Application Data
(60) Provisional application No. 60/484,481, filed on Jul. 1, 2003.

(51) Int. Cl. C12N 15/87; C12N 15/82; C07H 21/04

(52) U.S. Cl. 800/285; 536/23.1

(57) ABSTRACT

The present invention generally relates to the production and expression of microRNA (miRNA) in plants. In some cases, production and expression of miRNA can be used to at least partially inhibit or alter gene expression in plants. For instance, in some embodiments, a nucleotide sequence, which may encode a sequence substantially complementary to a gene to be inhibited or otherwise altered, may be prepared and inserted into a plant cell. Expression of the nucleotide sequence may cause the formation of precursor miRNA, which may, in turn, be cleaved (for example, with Dicer or other nucleases, including, for example, nucleases associated with RNA interference), to produce an miRNA sequence substantially complementary to the gene. The miRNA sequence may then interact with the gene (e.g., complementary binding) to inhibit the gene. In some cases, the nucleotide sequence may be an isolated nucleotide sequence. Other embodiments of the invention are directed to the precursor miRNA and/or the final miRNA sequence, as well as methods of making, promoting, and use thereof.

### RELATED APPLICATIONS

This application claims the benefit of U.S. Provisional Patent Application Ser. No. 60/484,481, filed Jul. 1, 2003, entitled “Micro RNAs in Plants,” by Reinhart, et al., incorporated herein by reference in its entirely.

### GOVERNMENT FUNDING

Certain aspects of the invention were developed using government funding under NIH RO1 HG02439. The Government may have certain rights in the invention.

### FIELD OF THE INVENTION

The present invention generally relates to the production and expression of microRNA (miRNA) in plants.

### BACKGROUND OF THE INVENTION

Although most genes use RNA in the form of mRNA as a coding intermediate for protein production, there are many genes whose final products are RNA. These noncoding RNAs range from the familiar transfer and ribosomal RNAs, to the more recently discovered regulatory RNAs. One type of regulatory RNA was first discovered during the study of nematode larval development. Two approximately 22-nucleotide (“nt”) RNAs (the lin-4 and let-7 RNAs) control developmental timing by binding to their respective mRNA targets and preventing productive use of these messages, perhaps by attenuating translation. The let-7 RNA was found broadly throughout bilateral animals, including humans, suggesting that these two riboregulators were more than oddities of worm larval development.

When these RNAs are expressed, they pair to sites within the 3′ untranslated region (“UTR”) of target mRNAs, triggering the translational repression of the mRNA targets. The mature lin-4 and let-7 RNAs are processed from the double-stranded region of RNA precursor transcripts by Dicer, a molecule with an N-terminal helicase and tandem C-terminal ribonuclease III domains. Argonaute homologs also influence the accumulation of the lin-4 and let-7 RNAs, but their biochemical roles are unclear. Argonaute family members have a PAZ domain, which may allow protein-protein interaction with Dicer, as well as a Piwi domain, whose function is unknown.

In 2001, it was discovered that these two RNAs are members of a large class of 21- to 24-nucleotide noncoding RNAs, called microRNAs (“miRNAs”), found in nematodes, fruitflies (Drosophila melanogaster), and humans. The abundance of the miRNA genes, their intriguing expression patterns in different tissues or in different stages of development, and their evolutionary conservation imply that, as a class, miRNAs have broad regulatory functions in addition to the known roles of lin-4 and let-7 RNAs in the temporal control of developmental events.

MicroRNAs are not the only small RNAs processed by Dicer. Dicer was originally identified as a nuclease involved in the RNA interference (“RNAi”) pathway of animals. This method of RNA silencing is triggered by long double-stranded RNA (“dsRNA”), typically introduced by injection or expression from a transgene. The dsRNA trigger is cleaved by Dicer into approximately 22-nucleotide RNAs. These nucleotide RNAs, known as small interfering RNAs (“siRNAs”), act as guide RNAs to target homologous mRNA sequences for destruction. RNAs of approximately 25 nucleotides in length are also associated with posttranscriptional gene silencing (“PTGS”) in plants, and it has been suggested that Dicer-like activity also produces these small RNAs.

### SUMMARY OF THE INVENTION

The present invention generally relates to the production, expression, and activity of microRNA (miRNA) in plants. The subject matter of the present invention involves, in some cases, interrelated products, alternative solutions to a particular problem, and/or a plurality of different uses of one or more systems and/or articles.

One aspect of the invention provides a composition. In one set of embodiments, the composition includes an isolated nucleotide sequence able to be transcribed by a plant cell into precursor miRNA that is cleavable by the plant cell to produce miRNA substantially complementary to at least a portion of an mRNA sequence encoding a gene. In another set of embodiments, the composition includes an isolated precursor miRNA able to inhibit a gene in a plant cell. The composition, in yet another set of embodiments, includes isolated plant-derived miRNA.

Another aspect of the invention provides a method of inhibiting a gene. The method, according to one set of embodiments, includes acts of replacing at least a portion of a nucleotide sequence, able to be transcribed by a plant cell into precursor miRNA cleavable by the plant cell to produce miRNA, with a sequence substantially complementary to a gene to be inhibited, and contacting the plant cell with the nucleotide to inhibit gene expression. According to another set of embodiments, the method includes acts of replacing a portion of a precursor miRNA taken from a plant cell with a sequence substantially complementary to a gene to be inhibited, and contacting the plant cell with the precursor miRNA to inhibit gene expression. In one set of embodiments, the method includes an act of altering expression of miRNA in a plant cell by altering an environmental condition surrounding the plant cell.

In another aspect, the present invention is directed to a method of making one or more of the embodiments described herein. In yet another aspect, the present invention is directed to a method of using one or more of the embodiments described herein. In still another aspect, the present invention is directed to a method of promoting one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more applications incorporated by reference include conflicting and/or inconsistent disclosure with respect to each other, then the later-filed application shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIGS. 1A-1KK are the structures of various precursor miRNAs cloned from Arabidopsis, according to one embodiment of the invention;

FIG. 2 is a table of miRNAs cloned from Arabidopsis, according to one embodiment of the invention;

FIGS. 3A-3BB are the structures of various precursor miRNAs cloned from Oryza, according to another embodiment of the invention;

FIG. 4 illustrates certain secondary structures of predicted Arabidopsis miRNA precursors, in accordance with one embodiment of the invention;

FIG. 5 is a scanned image of RNA blots demonstrating developmental expression of Arabidopsis miRNAs, in one embodiment of the invention;

FIG. 6 is a scanned image of RNA blots demonstrating a dependency of the expression of miR169 on CARPEL FACTORY, in one embodiment of the invention;

FIGS. 7A-7B illustrate conservation between predicted Arabidopsis and Oryza stem-loop precursors, in one embodiment of the invention;

FIG. 8 is a schematic diagram showing a cluster of small RNAs derived from Chromosome 2 in Arabidopsis, according to another embodiment of the invention;

FIG. 9 is a bar graph depicting antisense hits between Arabidopsis miRNAs and annotated mRNAs, in accordance with one embodiment of the invention;

FIG. 10 is a table showing certain potential regulatory targets of Arabidopsis miRNAs;

FIGS. 11A-11B are schematic diagrams of sequence context of certain miRNA complementary sites, in accordance with certain embodiments of the invention;

FIG. 12 is a table of miRNA complementary sites in potential mRNA targets conserved between Arabidopsis and Oryza, in accordance with one embodiment of the invention;

FIGS. 13A-13B is a schematic diagram of a model for the biogenesis, action, and roles of miRNAs in plants, according to another embodiment of the invention;

FIGS. 14A-14L are photocopies of photographs of certain Arabidopsis mutants exhibiting various developmental defects, in an embodiment of the invention;

FIGS. 15A-15L are bar graphs indicating steady-state levels of miRNA targets, in accordance with another embodiment of the invention;

FIGS. 16A-16B are scanned image of RNA blots demonstrating miRNA accumulation in Arabidopsis, according to one embodiment of the invention;

FIGS. 17A-17E illustrate certain silent mutations in the miR168 complementary site of AGO1 mRNA of Arabidopsis, according to yet another embodiment of the invention;

FIGS. 18A-18U are photocopies of photographs of certain transformed Arabidopsis mutants, according to another embodiment of the invention;

FIGS. 19A-19E illustrate certain compensatory mutations in the MIR168a gene, as rescued using an embodiment of the invention;

FIGS. 20A-20C illustrate an example method of determining an miRNA sequence, and examples of sequences so determined, in accordance with one embodiment of the invention;

FIG. 21 is a table of miRNAs cloned from Arabidopsis, according to another embodiment of the invention;

FIG. 22A-22B are graphs illustrating the number of mRNAs substantially complementary to certain conserved miRNAs, in one embodiment of the invention;

FIG. 23 is a table of miRNAs cloned from Arabidopsis, according to yet another embodiment of the invention;

FIGS. 24A-24C are scanned images of RNA blots demonstrating certain miRNAs of an embodiment of the invention;

FIG. 25 illustrates certain miRNA targets, according to certain embodiments of the invention;

FIG. 26 illustrates targeting of certain miRNA and complementary miRNA, according to 1o one embodiment of the invention;

FIG. 27 is a table illustrating the sensitivity of computational identification of certain miRNA, according to one embodiment of the invention;

FIGS. 28A-28G are tables illustrating certain miRNA found in Arabidopsis, according to one embodiment of the invention;

FIGS. 29A-29I are tables illustrating certain miRNA found in Oryza, according to another embodiment of the invention;

FIGS. 30A-30B illustrate MIR395 clustering in Arabidopsis and Oryza, according to certain embodiments of the invention;

FIGS. 31A-31O illustrate certain miRNA found in various species, according to certain embodiments of the invention; and

FIGS. 32A-32N are tables illustrating certain miRNA found in various species, according to various embodiments of the invention.

### BRIEF DESCRIPTION OF THE SEQUENCES

SEQ ID NO: 1 is UGACAGAAGAGAGUGAGCAC, an miRNA sequence arising from Arabidopsis thaliana;

SEQ ID NO: 2 is UUGACAGAAGAUAGAGAGCAC, an miRNA sequence arising from A. thaliana;

SEQ ID NO: 3 is UCCCAAAUGUAGACAAAGCA, an miRNA sequence arising from A. thaliana;

SEQ ID NO: 4 is UUUGGAUUGAAGGGAGCUCUA, an miRNA sequence arising from A. thaliana;

SEQ ID NO: 5 is UGCCUGGCUCCCUGUAUGCCA, an miRNA sequence arising from A. thaliana;

SEQ ID NO: 6 is UUGAAAGUGACUACAUCGGGG, an miRNA sequence arising from A. thaliana;

SEQ ID NO: 7 is UCGAUAAACCUCUGCAUCCAG, an miRNA sequence arising from A. thaliana;

SEQ ID NO: 8 is UUGAAGAGGACUUGGAACUUCGAU, an miRNA sequence arising from A. thaliana;

SEQ ID NO: 9 is UGGAGAAGCAGGGCACGUGCA, an miRNA sequence arising from A. thaliana;

SEQ ID NO: 10 is UCGGACCAGGCUUCAUCCCCC, an miRNA sequence arising from A. thaliana;

SEQ ID NO: 11 is UCGGACCAGGCUUCAUUCCCC, an miRNA sequence arising from A. thaliana;

SEQ ID NO: 12 is UGAAGCUGCCAGCAUGAUCUA, an miRNA sequence arising from A. thaliana;

SEQ ID NO: 13 is UCGCUUGGUGCAGGUCGGGAA, an miRNA sequence arising from A. thaliana;

SEQ ID NO: 14 is CAGCCAAGGAUGACUUGCCGA, an miRNA sequence arising from A. thaliana;

SEQ ID NO: 15 is UGAUUGAGCCGUGUCAAUAUC, an miRNA sequence arising from A. thaliana;

SEQ ID NO: 16 is UGAUUGAGCCGCGCCAAUAUC, an miRNA sequence arising from A. thaliana;

SEQ ID NO: 17 is ACAAAGGCAAUUUGCAUAUCAUUGCACUUGCUUCUCUUGC, an RNA fragment encoding a stem-loop motif, the fragment arising from A. thaliana;

SEQ ID NO: 18 is AUGCAGGCACUGUUAUGUGUCUAUAACUUUGCGUGUGC, an RNA fragment encoding a stem-loop motif, the fragment arising from A. thaliana;

SEQ ID NO: 19 is ACAAAGGCACUUUGCAUGUUCGAUGCAUUUGCUUCUCUUGC, an RNA fragment encoding a stem-loop motif, the fragment arising from A. thaliana;

SEQ ID NO: 20 is ACAAAGGGGAAGUUGUAUAAAAGUUUUGUAUAUGGUUGCUUUUGC, an RNA fragment encoding a stem-loop motif, the fragment arising from A. thaliana;

SEQ ID NO: 21 is ACAUGGUGGUUUCUUGCAUGCUUUUUUGAUUAGGGUUUCAUGCUUGAAGCUAUG U, an RNA fragment encoding a stem-loop motif, the fragment arising from A. thaliana;

SEQ ID NO: 22 is ACAUGGUGGCUUUCUUGCAUAUUUGAAGGUUCCAUGCUUGAAGCUAUGU, an RNA fragment encoding a stem-loop motif, the fragment arising from A. thaliana;

SEQ ID NO: 23 is AGAUGAUGAGAUACAAUUCGGAGCAUGUUCUUUGCAUCUUACUCCUUU, an RNA fragment encoding a stem-loop motif, the fragment arising from A. thaliana;

SEQ ID NO: 24 is AGAUGAUAAGAUACAAUUCCUCGCAGCUUCUUUGCAUCUUACUCCUUU, an RNA fragment encoding a stem-loop motif, the fragment arising from A. thaliana;

SEQ ID NO: 25 is UAAGGAUGACAUGCAAGUACAUACAUAUAUAUCAUCACACCGCAUGUGGAUGAU AAAAUAUGUAUAACAAAUUCAAAGAAAGAGAGGGAGAGAAAGAGAGAGAACCUG CAUCUCUACUCUUUU, an RNA fragment encoding a stem-loop motif, the fragment arising from A. thaliana;

SEQ ID NO: 26 is UAAGGAUGCUAUUGCAAAACAGACACAGAUAUGUGUUUCUAAUUGUAUUCAUAC UUUAACCUCAAAGUUGAUAUAAAAAAAGAAAGAAAGAUAGAAGAGCUAGAAGAC UAUCUGCAUCUCUAUUCCUAU, an RNA fragment encoding a stem-loop motif, the fragment arising from A. thaliana;

SEQ ID NO: 27 is AAAAGUGAUGACGCCAUUGCUCUU, an RNA fragment encoding a stem-loop motif, the fragment arising from A. thaliana;

SEQ ID NO: 28 is CAUGAGUUGAGCAGGGUAAAGAAAAGCUGCUAAGCUAUGGAUCCCAUAAGCCCU AAUCCUUGUAAAGUAAAAAAGGAUUUGGUUAUAUGGAUUGCAUAUCUCAGGAGC UUUAACUUGCCCUUUAAUGGCUUUUACUCUUC, an RNA fragment encoding a stem-loop motif, the fragment arising from A. thaliana;

SEQ ID NO: 29 is UAUGCUGAGCCCAUCGAGUAUCGAUGACCUCCGUGGA, an RNA fragment encoding a stem-loop motif, the fragment arising from A. thaliana;

SEQ ID NO: 30is CAAGAAAACAUCGAUUUAGUUUCAAAAUCGAUCACUAG, an RNA fragment encoding a stem-loop motif, the fragment arising from A. thaliana;

SEQ ID NO: 31 is CGAGUGGAUACCGAUUUUGGUUUUAAAAUCGGCUGCCGG, an RNA fragment encoding a stem-loop motif, the fragment arising from A. thaliana;

SEQ ID NO: 32is UUCCGAUUUUUUUUGUUCUUCAUAUGAUGAAGCGGAAACAGUAAUCAA, an RNA fragment encoding a stem-loop motif, the fragment arising from A. thaliana;

SEQ ID NO: 33 is UCUCUUCCUGUGAACACAUUAAAAAUGUAAAAGCAUGAAUAGA, an RNA fragment encoding a stem-loop motif, the fragment arising from A. thaliana;

SEQ ID NO: 34is UCAAUUCCUGUGAAUAUUUAUUUUUGUUUACAAAAGCAAGAAUCGA, an RNA fragment encoding a stem-loop motif, the fragment arising from A. thaliana;

SEQ ID NO:35 is CGACAACGAUUUCAACACUCUCUUCCAGGAACAACUUCCUCCAGGCAGAUGAUAC UAAAGUGCUGGAGUUCCCGGUUCCUGAGAGUGAGUCCAUAUCAAAAUGCGCAUU CGUUAUCACUUGGUUGAACCCAUUUGGGGAUUUAAAUUUGGAGGUGAAAUGGAA CGCGUAAUUGAUGACUCCUACGUGGAACCUCUUCUUAGGAAGAGCACGGUCGAA GAGUAACUGCGCAGUGCUUAAAUCGUAGAUGCUAAAGUCG, an RNA fragment encoding a stem-loop motif, the fragment arising from A. thaliana;

SEQ ID NO: 36 is AACCAACAAACACGAAAUCCGUCUCAUUUGCUUAUU, an RNA fragment encoding a stem-loop motif, the fragment arising from A. thaliana;

SEQ ID NO: 37 is UUACUAGCUCAUAUAUACACUCUCACCAUAAAUGCGUGUAUAUAUGCGGAAUUU UGUGAUAUAGAUGUGUGUGUGUGUUGAGUGUGAUGAUAUGGAUGAGUUAGUUC, an RNA fragment encoding a stem-loop motif, the fragment arising from A. thaliana;

SEQ ID NO: 38 is GGAUAUUAUAGAUAUAUACAUGUGUAUGUUAAUGAUUCAAGUGAUCAUAGAGA GUAUCC, an RNA fragment encoding a stem-loop motif, the fragment arising from A. thaliana;

SEQ ID NO: 39 is GGAUAUCAUAAACGCAUACACAUGUUUAUAUGUUAUGAUGCAUUAUAUGACUGA UGUAAUGUACAUAUAUAUACAUACAUGCCACAUGGUAUCG, an RNA fragment encoding a stem-loop motif, the fragment arising from A. thaliana;

SEQ ID NO: 40 is GGACUCUGGCUCGCUCUAUUCAUGUUGGAUCUCUUUCGAUCUAACAAUCGAAUU GAACCUUCAGAUUUCAGAUUUGAUUAGGGUUUUAGCGUCU, an RNA fragment encoding a stem-loop motif, the fragment arising from A. thaliana;

SEQ ID NO: 41 is GGACUCUUAUUCUAAUACAAUCUCAUUUGAAUACAUUCAGAUCUGAUGAUUGAU UAGGGUUUUAGUGUCG, an RNA fragment encoding a stem-loop motif, the fragment arising from A. thaliana;

SEQ ID NO: 42 is GGUCAUGAAGAAGAGAAUCACUCGAAUUAAUUUGGAAGAACAAAUUAAGAAAAC CCUAGAUGAUUC, an RNA fragment encoding a stem-loop motif, the fragment arising from A. thaliana;

SEQ ID NO: 43 is GGUCAUGAAGAAGAUCGGUAGAUUGAUUCAUUUUAAAGAGUGAAAUCCCUAAAU GAUUC, an RNA fragment encoding a stem-loop motif, the fragment arising from A. thaliana;

SEQ ID NO: 44 is GGCCCUUAACUUAGAUCUAUAUUUGAUUAUAUAUAUAUGUCUCUUCUUUAUUCA UUAGUCUAUACAUGAAUGAUCAUUUUACGGUUAAUGACG, an RNA fragment encoding a stem-loop motif, the fragment arising from A. thaliana;

SEQ ID NO: 45 is GACCAUUCAAUCUCAUGAUCUCAUGAUUAUAACGAUGAUGAUGAUGAUG, an RNA fragment encoding a stem-loop motif, the fragment arising from A. thaliana;

SEQ ID NO: 46 is GGUCAUGGAGAGUAAUUCGUUAACCCAACUCAAAACUCUAAAUGAUUC, an RNA fragment encoding a stem-loop motif, the fragment arising from A. thaliana;

SEQ ID NO: 47 is AUUAGCUUUCUUUAUCCUUUGUUGUGUUUCAUGACGAUGGUUAAGAGAUCAGUC UCGAU, an RNA fragment encoding a stem-loop motif, the fragment arising from A. thaliana;

SEQ ID NO: 48 is UCUUUGGUUAAGAGAUGAAUGUGGAAACAUAUUGCUUAAACCCAAGC, an RNA fragment encoding a stem-loop motif, the fragment arising from A. thaliana;

SEQ ID NO: 49 is CCAAUUCGGCUGACACAGCCGACUUUUAAACCUUUAUUGGUUUGUGAGCAUGGU CGGAUUGG, an RNA fragment encoding a stem-loop motif, the fragment arising from A. thaliana;

SEQ ID NO: 50 is CUGAUUGGCUGACACCGACACGUGUCUUGUCAUGGUUGGUUUGUGAGC, an RNA fragment encoding a stem-loop motif, the fragment arising from A. thaliana;

SEQ ID NO: 51 is UUUAAAUGAUCUUUCUUUAUACUCUAUUAAGACAAUUUAGUUUCAAACUUUUUU UUUUUUUUUUUUUUGAAGGAUUCAGGAAGAAAUUAGGAUAUAUUAUUCCGUAU AAAAUACAAGAUAUAUAAAACCAAAAGAAAAAGUAACAUGA, an RNA fragment encoding a stem-loop motif, the fragment arising from A. thaliana;

SEQ ID NO: 52 is GAUUCUCUUUUAUCAACUCAUC, an RNA fragment encoding a stem-loop motif, the fragment arising from A. thaliana;

SEQ ID NO: 53 is GAUCUUACCUGACCACACACGUAGAUAUACAUUAUUCUCUCUAGAUUAUC, an RNA fragment encoding a stem-loop motif, the fragment arising from A. thaliana;

SEQ ID NO: 54 is UGACAGAAGAGAGUGAGCAC, an miRNA sequence arising from Oryza sativa;

SEQ ID NO: 55 is UGCCUGGCUCCCUGUAUGCCA, an miRNA sequence arising from O. sativa;

SEQ ID NO: 56 is UCGAUAAACCUCUGCAUCCAG, an miRNA sequence arising from O. sativa;

SEQ ID NO: 57 is UGGAGAAGCAGGGCACGUGCA, an miRNA sequence arising from O. sativa;

SEQ ID NO: 58 is UCGGACCAGGCUUCAUUCCCC, an miRNA sequence arising from O. sativa;

SEQ ID NO: 59 is UGAAGCUGCCAGCAUGAUCUA, an miRNA sequence arising from O. sativa;

SEQ ID NO: 60 is CAGCCAAGGAUGACUUGCCGA, an miRNA sequence arising from O. sativa;

SEQ ID NO: 61 is UGAUUGAGCCGCGCCAAUAUC, an miRNA sequence arising from O. sativa;

SEQ ID NO: 62 is ACGUGGUUGUUUCCUUGCAUAAAUGAUGCCUAUGCUUGGAGCUACGC, an RNA fragment encoding a stem-loop motif, the fragment arising from O. sativa;

SEQ ID NO: 63 is ACACGGUGCUUUCUUAGCAUGCAAGAGCCAUGCUGGGAGCUGUGC, an RNA fragment encoding a stem-loop motif, the fragment arising from O. sativa;

SEQ ID NO: 64 is ACAUGGUGACUUUCUUGCAUGCUGAAUGGACUCAUGCUUGAAGCUAUGU, an RNA fragment encoding a stem-loop motif, the fragment arising from O. sativa;

SEQ ID NO: 65 is ACAGCGUGAUGGCCGGCAUAAAAUCUAUCCCGUCCUCGCCGC, an RNA fragment encoding a stem-loop motif, the fragment arising from O. sativa;

SEQ ID NO: 66 is ACGGCCGGGCGUGACGGCACCGGCGGGCGUGCCGUCGCGGCCGC, an RNA fragment encoding a stem-loop motif, the fragment arising from O. sativa;

SEQ ID NO: 67 is ACAGCGGCCAGACUGCAUCGAUCUAUCAAUCUUCCCUUCGACAGGAUAACUAGG UAGAAAGAAAGAGAGGCCGUCGGCGGCCAUGGAAGAGAOAGAGAAGAGAGAGAGA UGAAUGAUGAUGAUGAUACAGCUGCCGCUCGC, an RNA fragment encoding a stem-loop motif, the fragment arising from O. sativa;

SEQ ID NO:68 is ACAGCGGGCAGACUGCAUCUGAAAUAAACUGGUGACGACGAAGAAGACGACGGA CGCAGCUUGCCGUUGC, an RNA fragment encoding a stem-loop motif, the fragment arising from O. sativa;

SEQ ID NO: 69 is ACGGCGCGGCGGCUAGCCAUCGGCGGGAUGCCUGCCCCCGCCGC, an RNA fragment encoding a stem-loop motif, the fragment arising from O. sativa;

SEQ ID NO: 70 is CGCCGGCGCUGCCGUGUAGGCGGCCACGGCAAGGCGGGCCGGCA, an RNA fragment encoding a stem-loop motif, the fragment arising from O. sativa;

SEQ ID NO: 71 is CGCCGCCCCCGUCCGUAGGGCGGCUACCGAUCGGCGGCGCGGCA, an RNA fragment encoding a stem-loop motif, the fragment arising from O. sativa;

SEQ ID NO: 72 is CACAUGUAGACCAACCCAUGGUGUCUGGUUGCCUACUGGG, an RNA fragment encoding a stem-loop motif, the fragment arising from O. sativa;

SEQ ID NO: 73 is CUCAUGUAGCCCAAUCCAUGGUGUGUUUGGAUGCUGUGGG, an RNA fragment encoding a stem-loop motif, the fragment arising from O. sativa;

SEQ ID NO: 74 is CUCAUCUAGAGCAACAAACUUCUGCGAGAGGUUGCCUAUGAUGGA, an RNA fragment encoding a stem-loop motif, the fragment arising from O. sativa;

SEQ ID NO: 75 is CUCGCGUAGCUGCCAAACUCAGUUGAAACAACUGCCUUCUCCCGGCGAGAUUCAG GCAUUGUGUUCGUACGUUUGGCUCUACUGCGGA, an RNA fragment encoding a stem-loop motif, the fragment arising from O. sativa;

SEQ ID NO: 76 is UCCCUUCCCUGCCUUGUGGCGCUGAUCCAGGAGCGGCGAAUUUCUUUGAGAGGG UGUUCUUUUUUUUUUCUUCCUUUUGGUCCUUGUUGCAGCCAACGACAACGCGGG AAUCGA, an RNA fragment encoding a stem-loop motif, the fragment arising from O. sativa;

SEQ ID NO: 77 is UGCAUAUGUUCAUCAUCAUCUUCUUCCUCCUCCUCUAGCUCCAGCCUUGUGUGGG UUGGAAGUUUAGAUAGAACUCGCA, an RNA fragment encoding a stem-loop motif, the fragment arising from O. sativa;

SEQ ID NO: 78 is UUACCAUCCACUCGCCUGCCGGCCGCCGGCCGCCAUUGCCAUGGAUGGUUC, an RNA fragment encoding a stem-loop motif, the fragment arising from O. sativa;

SEQ ID NO: 79 is GGUCUCAUACACCUUGUGGUUUUGAGGAUGAUUUGUGCAAGGUUUUUCAUUCCU CUCAUCCGUGGGAUC, an RNA fragment encoding a stem-loop motif, the fragment arising from O. sativa;

SEQ ID NO: 80 is GGCUACUUUUAAUUUCUCUCUCUUUUGAUAUCUUCUUUUCUCGAUCUCCUAGCU UGAUCYUUUUGAUCUCUCAAAUCGAUCUUAAGAAAAAGAUCAGUCAAAGAGAUG AGAGUAGAUGUCUGUAGAUC, an RNA fragment encoding a stem-loop motif, the fragment arising from O. sativa;

SEQ ID NO: 81 is GACCUAACACCGGGCGGAAUGGCGGAUUCAGCUGCAGCUAAGCAAGCUAGGUGG GGGGUU, an RNA fragment encoding a stem-loop motif, the fragment arising from O. sativa;

SEQ ID NO: 82 is GGUGCAUGGAGAAACCUCUGAUCGAUCAGGUUUGAUCUGUAGAGACUGAUC, an RNA fragment encoding a stem-loop motif, the fragment arising from O. sativa;

SEQ ID NO: 83 is GGCCCCUUAGGAUGUGUGAUUUUUGAUGGUUUAUGCAUUCAUCUUGAUGCGAAC AUCUAUCUCGGAUCUUUGGGUUC, an RNA fragment encoding a stem-loop motif, the fragment arising from O. sativa;

SEQ ID NO: 84 is GAUCGUACCAUAGUGGUGGGUACACGUGGACGGUC, an RNA fragment encoding a stem-loop motif, the fragment arising from O. sativa;

SEQ ID NO:85is GCUCUGAUUAAUCGGCACUGUUGGCGUACAGUCGAUUGACUAAUCGUCAGAUCU GUGUGUGUAAAUCACUGU, an RNA fragment encoding a stem-loop motif, the fragment arising from O. sativa;

SEQ ID NO: 86is ACUUGCAGACAAGAAAUCAGCUCAGCUCGCUGGUUUCGAACAGGAAGGCGGCUA GCUGAGGCUUCUUCUGAGUACGUGAUGGU, an RNA fragment encoding a stem-loop motif, the fragment arising from O. sativa;

SEQ ID NO: 87is GCUCUGAAUGAUCAACAAGAUGUGCUCCCACACUGCCUUCCUGUGGAUCUUGAG CUGUUGCUAGUCUUGUGGUCAUGCCUUGC, an RNA fragment encoding a stem-loop motif, the fragment arising from O. sativa;

SEQ ID NO: 88 is UCGAUCGAUCUAUCUAUGAAGCUAAGCUAGCUGGCCAUGGAUCCAUCCAUCAA, an RNA fragment encoding a stem-loop motif, the fragment arising from O. sativa;

SEQ ID NO: 89 is GAUGAUUGGYUUUACAGCAGUGGUAAAAUCAGUAUC, an RNA fragment encoding a stem-loop motif, the fragment arising from O. sativa;

SEQ ID NO: 90 is UUCAAUAAAUAAUUGGUUCUA, an RNA fragment arising from Chromosome 2 of A. thaliana;

SEQ ID NO: 91 is GAACUAGAAAAGACAUUGGAC, an RNA fragment arising from Chromosome 2 of A. thaliana;

SEQ ID NO: 92 is UCCAAUGUCUUUUCUAGUUCGU, an RNA fragment arising from Chromosome 2 of A. thaliana;

SEQ ID NO: 93 is AGAGUAAGAUGGAUCUUGAUAA, an RNA fragment arising from Chromosome 2 of A. thaliana;

SEQ ID NO: 94 is UAUAUCCCAUUUCUACCAUCUG, an RNA fragment arising from Chromosome 2 of A. thaliana;

SEQ ID NO: 95 is UCCAAGCGAAUGAUGAUACUU, an RNA fragment arising from Chromosome 2 of A. thaliana;

SEQ ID NO: 96 is TLAEFLSKATGTAVDWVQMPGMKPGPDSVGIFAISQRCNGVAARAC, an miR165 complementary site that lies within the START domain of HDZip transcription factor REV in A. thaliana;

SEQ ID NO: 97 is TLTEFISKATGTAVEWVQMPGMKPGPDSIGIVAISHGCTGIAARAC, an miR165 complementary site that lies within the START domain of HDZip transcription factor ATHB-8 in A. thaliana;

SEQ ID NO: 98 is TLAEFLCKATGTAVDWVQMIGMKPGPDSIGIVAVSRNCSGIAARAC, an miR165 complementary site that lies within the START domain of HDZip transcription factor PHV in A. thaliana;

SEQ ID NO: 99 is ALAEFLSKATGTAVDWVQMIGMKPGPDSIGIVAISRNCSGIAARAC, an miR165 complementary site that lies within the START domain of HDZip transcription factor PHB in A. thaliana;

SEQ ID NO: 100 IS SSRTASLCERMTSCIHDSDCALSLLSSSSSSVPHLLQPPLSLSQEA, a fragment of a protein encoded by gene At5g50570, containing an miR156 complementary site, found in A. thaliana;

SEQ ID NO: 101 IS SSRTASLCERMTSCIHDSDCALSLLSSSSSSVPHLLQPPLSLSQEA, a fragment of a protein encoded by gene At5g50670, containing an miR156 complementary site, found in A. thaliana;

SEQ ID NO: 102 IS PDKGVGECSGGLHESHDFYSALSLLSTTSDSQGIKHTPVAEPPPIF, a fragment of a protein encoded by gene At1g27370, containing an miR156 complementary site, found in A. thaliana; SEQ ID NO: 103 IS HGEDVGEYSGVLHESQDIHRALSLLSTSSDPLAQPHVQPFSLLCSY, a fragment of a protein encoded by gene At1g27360, containing an miR156 complementary site, found in A. thaliana;

SEQ ID NO: 104 IS FSKEKVTISSSHMGASQDLDGALSLLSNSTTWVSSSDQPRRFTLDHH, a fragment of a protein encoded by gene At5g43270, containing an miR156 complementary site, found in A. thaliana;

SEQ ID NO: 105 is SSSFTTCPEMINNNSTDSSCALSLLSNSYPIHQQQLQTPTNTWRPS, a fragment of a protein encoded by gene At3g57920, containing an miR156 complementary site, found in A. thaliana;

SEQ ID NO: 106 is SPEIMDTKLESYKGIGDSNCALSLLSNPHQPHDNNNNNNNNNNNNN, a fragment of a protein encoded by gene At2g42200, containing an miR156 complementary site, found in A. thaliana;

SEQ ID NO: 107 is STEVSSIWDLHETAASRSTRALSLLSAQSQQHLSKFPNTTFSITQP, a fragment of a protein encoded by gene At1g69170, containing an miR156 complementary site, found in A. thaliana;

SEQ ID NO: 108 is UGUGCUCACUCUCUUCUGUCA, an RNA sequence complementary to miR156; SEQ ID NO: 109 is UGUGCUCUCUCUCUUCUGUCA, an RNA fragment of gene At5g50570, containing a site substantially complementary to miR156;

SEQ ID NO: 110 is UGUGCUCUCUCUCUUCUGUCA, an RNA fragment of gene At5g50670, containing a site substantially complementary to miR156;

SEQ ID NO: 111 is UGUGCUCUCUCUCUUCUGUCA, an RNA fragment of gene At3g57920, containing a site substantially complementary to miR156;

SEQ ID NO: 112 is UGUGCUCUCUCUCUUCUGUCA, an RNA fragment of gene At2g42200, containing a site substantially complementary to miR156;

SEQ ID NO: 113 is AGUGCUCUCUCUCUUCUGUCA, an RNA fragment of gene At1g27370, containing a site substantially complementary to miR156;

SEQ ID NO: 114 is CGUGCUCUCUCUCUUCUGUCA, an RNA fragment of gene At1g27360, containing a site substantially complementary to miR156;

SEQ ID NO: 115 is GGUGCUCUCUCUCUUCUGUCA, an RNA fragment of gene At5g43270, containing a site substantially complementary to miR156;

SEQ ID NO: 116 is CGUGCUCUCUCUCUUCUGUCA, an RNA fragment of gene At1g69170, containing a site substantially complementary to miR156;

SEQ ID NO: 117 is UUUGCUUACUCUCUUCUGUCA, an RNA fragment of gene At2g33810, containing a site substantially complementary to miR156;

SEQ ID NO: 118 is UCUGCUCUCUCUCUUCUGUCA, an RNA fragment of gene At1g53160, containing a site substantially complementary to miR156;

SEQ ID NO: 119 is UGUGCUCUCUCUCUUCUGUCA, an RNA fragment of gene Os 20095, containing a site substantially complementary to miR156;

SEQ ID NO: 120 is UGUGCUCUCUCUCUUCUGUCA, an RNA fragment of gene Os 06618, containing a site substantially complementary to miR156;

SEQ ID NO: 121 is UGUGCUCUCUCUCUUCUGUCA, an RNA fragment of gene Os 02878, containing a site substantially complementary to miR156;

SEQ ID NO: 122 is GGUGCUCUCUCUCUUCUGUCA, an RNA fragment of gene Os 25470, containing a site substantially complementary to miR156;

SEQ ID NO: 123 is CALSLLS, a protein fragment encoded by At5g50570;

SEQ ID NO: 124 is CALSLLS, a protein fragment encoded by At5g50670;

SEQ ID NO: 125 is CALSLLS, a protein fragment encoded by At3g57920;

SEQ ID NO: 126 is CALSLLS, a protein fragment encoded by At2g42200;

SEQ ID NO: 127 is SALSLLS, a protein fragment encoded by At1g27370;

SEQ ID NO: 128 is RALSLLS, a protein fragment encoded by At1g27360;

SEQ ID NO: 129 is GALSLLS, a protein fragment encoded by At5g43270;

SEQ ID NO: 130 is RALSLLS, a protein fragment encoded by At1g69170;

SEQ ID NO: 131 is CALSLLS, a protein fragment encoded by Os 20095;

SEQ ID NO: 132 is CALSLLS, a protein fragment encoded by Os 06618;

SEQ ID NO: 133 is CALSLLS, a protein fragment encoded by Os 02878;

SEQ ID NO: 134 is GALSLLS, a protein fragment encoded by Os 25470;

SEQ ID NO: 135 is UGGCAUACAGGGAGCCAGGCA, an RNA sequence complementary to miR160;

SEQ ID NO: 136 is UGGCAUGCAGGGAGCCAGGCA, an RNA fragment of gene At1g77850, containing a site substantially complementary to miR160;

SEQ ID NO: 137 is AGGAAUACAGGGAGCCAGGCA, an RNA fragment of gene At2g28350, containing a site substantially complementary to miR160;

SEQ ID NO: 138 is GGGUUUACAGGGAGCCAGGCA, an RNA fragment of gene At4g30080, containing a site substantially complementary to miR160;

SEQ ID NO: 139 is AGGCAUACAGGGAGCCAGGCA, an RNA fragment of gene OsTC73519, containing a site substantially complementary to miR160;

SEQ ID NO: 140 is AGGCAUACAGGGAGCCAGGCA, an RNA fragment of gene OsTC7063 1, containing a site substantially complementary to miR160;

SEQ ID NO: 141 is AGGCAUACAGGGAGCCAGGCA, an RNA fragment of gene Os 17478, containing a site substantially complementary to miR160;

SEQ ID NO: 142 is AGGCAUACAGGGAGCCAGGCA, an RNA fragment of gene Os 02679, containing a site substantially complementary to miR160;

SEQ ID NO: 143 is AGMQGARQ, a protein fragment encoded by At1g77850;

SEQ ID NO: 144 is AGIQGARQ, a protein fragment encoded by At2g28350;

SEQ ID NO: 145 is VGLQGARH, a protein fragment encoded by At4g30080;

SEQ ID NO: 146 is AGIQGARH, a protein fragment encoded by OsTC73519;

SEQ ID NO: 147 is AGIQGARH, a protein fragment encoded by OsTC7063 1;

SEQ ID NO: 148 is AGIQGARH, a protein fragment encoded by Os 17478;

SEQ ID NO: 149 is AGIQGARH, a protein fragment encoded by Os 02679;

SEQ ID NO: 150 is UGCACGUGCCCUGCUUCUCCA, an RNA sequence complementary to miR164;

SEQ ID NO: 151 is AGCACGUACCCUGCUUCUCCA, an RNA fragment of gene At1g56010, containing a site substantially complementary to miR164;

SEQ ID NO: 152 is UUUACGUGCCCUGCUUCUCCA, an RNA fragment of gene At5g07680, containing a site substantially complementary to miR164;

SEQ ID NO: 153 is UCUACGUGCCCUGCUUCUCCA, an RNA fragment of gene At5g61430, containing a site substantially complementary to miR164;

SEQ ID NO: 154 is AGCACGUGUCCUGUUUCUCCA, an RNA fragment of gene At3g15170, containing a site substantially complementary to miR164;

SEQ ID NO: 155 is AGCACGUGUCCUGUUUCUCCA, an RNA fragment of gene At5g53950, containing a site substantially complementary to miR164;

SEQ ID NO: 156 is CGCACGUGACCUGCUUCUCCA, an RNA fragment of gene Os 00116, containing a site substantially complementary to miR164;

SEQ ID NO: 157 is EHVPCFSN, a protein fragment encoded by At1g56010;

SEQ ID NO: 158 is VYVPCFSN, a protein fragment encoded by At5gO7680;

SEQ ID NO: 159 is VYVPCFSN, a protein fragment encoded by At5g61430;

SEQ ID NO: 160 is EHVSCFSN, a protein fragment encoded by At3g15170;

SEQ ID NO: 161 is EHVSCFST, a protein fragment encoded by At5g53950;

SEQ ID NO: 162 is AHVTCFSN, a protein fragment encoded by Os 00116;

SEQ ID NO: 163 is UAGAUCAUGCUGGCAGCUUCA, an RNA sequence complementary to miR167;

SEQ ID NO: 164 is UAGAUCAGGCUGGCAGCUUGU, an RNA fragment of gene At5g37020, containing a site substantially complementary to miR167;

SEQ ID NO: 165 is UAGAUCAGGCUGGCAGCUUGU, an RNA fragment of gene OsTC79868, containing a site substantially complementary to miR167;

SEQ ID NO: 166 is LRSGWQLV, a protein fragment encoded by At5g37020;

SEQ ID NO: 167 is DRSGWQLV, a protein fragment encoded by OsTC79868;

SEQ ID NO: 168 is UCGGCAAGUCAUCCUUGGCUG, an RNA sequence complementary to miR169;

SEQ ID NO: 169 is AAGGGAAGUCAUCCUUGGCUG, an RNA fragment of gene At1g17590, containing a site substantially complementary to miR169;

SEQ ID NO: 170 is ACGGGAAGUCAUCCUUGGCUA, an RNA fragment of gene At1g54160, containing a site substantially complementary to miR169;

SEQ ID NO: 171 is UAGGCAACUCAUUCUUGGCUG, an RNA fragment of gene Os 04048, containing a site substantially complementary to miR169;

SEQ ID NO: 172 is UAGGCAAUUCAUCCUUGGCUU, an RNA fragment of gene Os 09843, containing a site substantially complementary to miR169;

SEQ ID NO: 173 is GAUAUUGGCGCGGCUCAAUCA, an RNA sequence complementary to miR171;

SEQ ID NO: 174 is GAUAUUGGCGCGGCUCAAUCA, an RNA fragment of gene At2g45160, containing a site substantially complementary to miR171;

SEQ ID NO: 175 is GAUAUUGGCGCGGCUCAAUCA, an RNA fragment of gene At3g60630, containing a site substantially complementary to miR171;

SEQ ID NO: 176 is GAUAUUGGCGCGGCUCAAUCA, an RNA fragment of gene At4g00150, containing a site substantially complementary to miR171;

SEQ ID NO: 177 is GAUAUUGGCGCGGCUCAAUCA, an RNA fragment of gene OsTC76755, containing a site substantially complementary to miR171;

SEQ ID NO: 178 is GAUAUUGGCGCGGCUCAAUCA, an RNA fragment of gene OsTC81772, containing a site substantially complementary to miR171;

SEQ ID NO: 179 is GAUAUUGGCGCGGCUCAAUCA, an RNA fragment of gene Os 00711, containing a site substantially complementary to miR171;

SEQ ID NO: 180 is GAUAUUGGCGCGGCUCAAUCA, an RNA fragment of gene Os 12185, containing a site substantially complementary to miR171;

SEQ ID NO: 181 is GAUAUUGGCGCGGCUCAAUUA, an RNA fragment of gene OsTC75254, containing a site substantially complementary to miR171;

SEQ ID NO: 182 is GILARLNH, a protein fragment encoded by At2g45160;

SEQ ID NO: 183 is GILARLNH, a protein fragment encoded by At3g60630;

SEQ ID NO: 184 is GILARLNQ, a protein fragment encoded by At4g00150;

SEQ ID NO: 185 is EILARLNQ, a protein fragment encoded by OsTC76755;

SEQ ID NO: 186 is EILARLNH, a protein fragment encoded by OsTC81772;

SEQ ID NO: 187 is EILARLNQ, a protein fragment encoded by Os 00711;

SEQ ID NO: 188 is EILARLNQ, a protein fragment encoded by Os 12185;

SEQ ID NO: 189 is EILARLNY, a protein fragment encoded by OsTC7525;

SEQ ID NO: 190 is UUGGGAUGAAGCCUGGUCCGG, a fragment of PHV wild-type mRNA, containing an miR165/166 complementary region;

SEQ ID NO: 191 is UCGGACCAGGCUUCAUUCCCC, an mRNA sequence substantially complementary to SEQ ID NO: 190;

SEQ ID NO: 192 is UCGGACCAGGCUUCAUCCCCC, an mRNA sequence substantially complementary to SEQ ID NO: 190;

SEQ ID NO: 193 is CCGGACCAGGCUUCAUCCCAA, an mRNA sequence substantially complementary to SEQ ID NO: 190;

SEQ ID NO: 194 is CCGGAUCAGGCUUCAUCCCAA, an mRNA sequence substantially complementary to SEQ ID NO: 190;

SEQ ID NO: 195 is UUGGGAUGAAGCCUGAUCCGG, a fragment of PHV mRNA having a G→A mutation, containing an miR165/166 complementary region;

SEQ ID NO: 196 is GATCCATTCCTAAGCGAAGTTTCAGAG, a primer useful in PCR;

SEQ ID NO: 197 is GCCCGAGCAACATAAAGATCCATAG, a primer useful in PCR;

SEQ ID NO: 198 is AGACCTTGGCGGAGTTCCTTTG, a primer useful in PCR;

SEQ ID NO: 199 is GTTGCGTGAAACAGCTACGATACC, a primer useful in PCR;

SEQ ID NO: 200 is TCTTTCCCTGCTCAATGCTCCTC, a primer useful in PCR;

SEQ ID NO: 201 is TTTCGCCACTGTCTCTCCTCTAAC, a primer useful in PCR;

SEQ ID NO: 202 is CTGGAGGTTTTGAGGCTGGTAT, a primer useful in PCR;

SEQ ID NO: 203 is CCAAGGGTGAAAGCAAGAAGA, a primer useful in PCR;

SEQ ID NO: 204 is CTCGACTCTCGAGGTAGTATTAATTAACGAGTTCTAAGTTCTTCTTCCGTTATGAG, a primer useful in PCR;

SEQ ID NO: 205 is GGTTCTGGTACCTGGGTAGGACTCACCTCAGACAGTGTAGGCTGAGAAGACACCGC, a primer useful in PCR;

SEQ ID NO: 206 is CCACCGCAGAGACAATCAGTGCCGGAGCTCCATCAGGCTACCTCACCTACTTATCA AGCG, a primer useful in PCR;

SEQ ID NO: 207 is CGCTTGATAAGTAGGTGAGGTAGCCTGATGGAGCTCCGGCACTGATTGTCTCTGCG GTGG, a primer useful in PCR;

SEQ ID NO: 208 is CACCATCGGGCTCGGATTCGCCTGGTGGAGGTCCGGCACCAATTCGGCTGACACAG CC, a primer useful in PCR;

SEQ ID NO: 209 is GGCTGTGTCAGCCGAATTGGTGCCGGACCTCCACCAGGCGAATCCGAGCCCGATGG TG, a primer useful in PCR;

SEQ ID NO: 210 is TTGGTTTGTGAGCAGGGATTGGAGCCGGCCTrCCATCAGCTGAATCGGATCCTCGAG GTGTA, a primer useful in PCR;

SEQ ID NO: 211 is TACACCTCGAGGATCCGATTCAGCTGATGGAAGGCCGGCTCCAATCCCTGCTCACA AACCAA, a primer useful in PCR;

SEQ ID NO: 212 is GUUCCCGAGCUGCAUCAAGCUACC, an mRNA fragment of gene AGO1;

SEQ ID NO: 213 is VPELHQAT, a peptide fragment corresponding to SEQ ID NO: 212;

SEQ ID NO: 214 is UCGCUUGGUGCAGGUCGGGAA, an RNA fragment of miR168;

SEQ ID NO: 215 is GUUCCCGAGCUCCAUCAGGCUACC, an mRNA fragment of gene 2m-AG1;

SEQ ID NO: 216 is VPELHQAT, a peptide fragment corresponding to SEQ ID NO: 215;

SEQ ID NO: 217 is UCGCUUGGUGCAGGUCGGGAA, an RNA fragment of miR168;

SEQ ID NO: 218 is GUGCCGGAGCUCCAUCAGGCUACC, an mRNA fragment of gene 4m-AGO1;

SEQ ID NO: 219 is VPELHQAT, a peptide fragment corresponding to SEQ ID NO: 218;

SEQ ID NO: 220 is UCGCUUGGUGCAGGUCGGGAA, an RNA fragment of miR168;

SEQ ID NO: 221 is GUUCCCGAGCUGCAUCAAGCUACC, an mRNA fragment of gene AGO1;

SEQ ID NO: 222 is AAGGGCUGGACGUGGUUCCCU, an RNA fragment of miR168;

SEQ ID NO: 223 is UCGCUUGGUGCAGGUCGGGAA, a first portion of an RNA fragment of a MIR168 primary transcript;

SEQ ID NO: 224 is GAUCCCGCCUUGCAUCAACUGAAU, a second portion of an RNA fragment of a MIR168 primary transcript, substantially complementary to SEQ ID NO: 223;

SEQ ID NO: 225 is GUGCCGGAGCUCCAUCAGGCUACC, an mRNA fragment of gene 4m-AGO1;

SEQ ID NO: 226 is UCGCUUGGUGCAGGUCGGGAA, an RNA fragment of miR168;

SEQ ID NO: 227 is GUGCCGGAGCUCCAUCAGGCUACC, an mRNA fragment of gene 4m-AGO1;

SEQ ID NO: 228 is CGCCUGGUGGAGGUCCGGCA, an RNA fragment of a modified miR168, 4m-miR168;

SEQ ID NO: 229 is AUUCGCCUGGUGGAGGUCCGGCAG, a first portion of an RNA fragment of a 4m-MIR168 primary transcript;

SEQ ID NO: 230 is GAGCCGGCCUUCCAUCAGCUGAAU, a second portion of an RNA fragment of a 4m-MIR168 primary transcript, substantially complementary to SEQ ID NO: 229;

SEQ ID NO: 231 is UGACAGAAGAGAGUGAGCACA, an miRNA sequence arising from A. thaliana;

SEQ ID NO: 232 is UUGACAGAAGAUAGAGAGCAC, an miRNA sequence arising from A. thaliana;

SEQ ID NO: 233 is UCCCAAAUGUAGACAAAGCA, an miRNA sequence arising from A. thaliana;

SEQ ID NO: 234 is UUUGGAUUGAAGGGAGCUCUA, an miRNA sequence arising from A. thaliana;

SEQ ID NO: 235 is UUUGGAUUGAAGGGAGCUCUU, an miRNA sequence arising from A. thaliana;

SEQ ID NO: 236 is UUUGGAUUGAAGGGAGCUCCU, an miRNA sequence arising from A. thaliana;

SEQ ID NO: 237 is UGCCUGGCUCCCUGUAUGCCA, an miRNA sequence arising from A. thaliana;

SEQ ID NO: 238 is UUGAAAGUGACUACAUCGGGG, an miRNA sequence arising from A. thaliana;

SEQ ID NO: 239 is UCGAUAAACCUCUGCAUCCAG, an miRNA sequence arising from A. thaliana;

SEQ ID NO: 240 is UUGAAGAGGACUUGGAACUUCGAU, an miRNA sequence arising from A. thaliana;

SEQ ID NO: 241 is UGGAGAAGCAGGGCACGUGCA, an miRNA sequence arising from A. thaliana;

SEQ ID NO: 242 is UCGGACCAGGCUUCAUCCCCC, an miRNA sequence arising from A. thaliana;

SEQ ID NO: 243 is UCGGACCAGGCUUCAUUCCCC, an miRNA sequence arising from A. thaliana;

SEQ ID NO: 244 is UGAAGCUGCCAGCAUGAUCUA, an miRNA sequence arising from A. thaliana;

SEQ ID NO: 245 is UGAAGCUGCCAGCAUGAUCUG, an miRNA sequence arising from A. thaliana;

SEQ ID NO: 246 is UCGCUUGGUGCAGGUCGGGAA, an miRNA sequence arising from A. thaliana;

SEQ ID NO: 247 is CAGCCAAGGAUGACUUGCCGA, an miRNA sequence arising from A. thaliana;

SEQ ID NO: 248 is UGAUUGAGCCGUGUCAAUAUC, an miRNA sequence arising from A. thaliana;

SEQ ID NO: 249 is UGAUUGAGCCGCGCCAAUAUC, an miRNA sequence arising from A. thaliana;

SEQ ID NO: 250 is AGAAUCUUGAUGAUGCUGCAU, an miRNA sequence arising from A. thaliana;

SEQ ID NO: 251 is AGAAUCUUGAUGAUGCUGCAG, an miRNA sequence arising from A. thaliana;

SEQ ID NO: 252 is GGAAUCUUGAUGAUGCUGCAU, an miRNA sequence arising from A. thaliana;

SEQ ID NO: 253 is UUCGCUUGCAGAGAGAAAUCAC, an miRNA sequence arising from A. thaliana;

SEQ ID NO: 254 is UUGGACUGAAGGGAGCUCCC, an miRNA sequence arising from A. thaliana;

SEQ ID NO: 255 is UCCAAAGGGAUCGCAUUGAUC, an miRNA sequence arising from A. thaliana;

SEQ ID NO: 256 is UCCAAAGGGAUCGCAUUGAUC, an miRNA sequence arising from A. thaliana;

SEQ ID NO: 257 is UUCUUUGGCAUUCUGUCCACC, an miRNA sequence arising from A. thaliana;

SEQ ID NO: 258 is UUCUUUGGCAUUCUGUCCACC, an miRNA sequence arising from A. thaliana;

SEQ ID NO: 259 is CUGAAGUGUUUGGGGGAACUC, an miRNA sequence arising from A. thaliana;

SEQ ID NO: 260 is CUGAAGUGUUUGGGGGAACUC, an miRNA sequence arising from A. thaliana;

SEQ ID NO: 261 is CUGAAGUGUUUGGGGGAACUC, an miRNA sequence arising from A. thaliana;

SEQ ID NO: 262 is CUGAAGUGUUUGGGGGGACUC, an miRNA sequence arising from A. thaliana;

SEQ ID NO: 263 is CUGAAGUGUUUGGGGGGACUC, an miRNA sequence arising from A. thaliana;

SEQ ID NO: 264 is CUGAAGUGUUUGGGGGGACUC, an miRNA sequence arising from A. thaliana;

SEQ ID NO: 265 is UUCCACAGCUUUCUUGAACUG, an miRNA sequence arising from A. thaliana;

SEQ ID NO: 266 is UUCCACAGCUUUCUUGAACUU, an miRNA sequence arising from A. thaliana;

SEQ ID NO: 267 is UCAUUGAGUGCAGCGUUGAUG, an miRNA sequence arising from A. thaliana;

SEQ ID NO: 268 is UCAUUGAGUGCAUCGUUGAUG, an miRNA sequence arising from A. thaliana;

SEQ ID NO: 269 is UGUGUUCUCAGGUCACCCCUU, an miRNA sequence arising from A. thaliana;

SEQ ID NO: 270 is UGUGUUCUCAGGUCACCCCUG, an miRNA sequence arising from A. thaliana;

SEQ ID NO: 271 is UGUGUUCUCAGGUCACCCCUG, an miRNA sequence arising from A. thaliana;

SEQ ID NO: 272 is UGCCAAAGGAGAUUUGCCCUG, an miRNA sequence arising from A. thaliana;

SEQ ID NO: 273 is CCUGCCAAAGGAGAGUUGCCCUG, an miRNA sequence arising from A. thaliana;

SEQ ID NO: 274 is CCUGCCAAAGGAGAGUUGCCCUG, an miRNA sequence arising from A. thaliana;

SEQ ID NO: 275 is UGCCAAAGGAGAUUUGCCCCG, an miRNA sequence arising from A. thaliana;

SEQ ID NO: 276 is UGCCAAAGGAGAUUUGCCUCG, an miRNA sequence arising from A. thaliana;

SEQ ID NO: 277 is UGCCAAAGGAGAUUUGCCCGG, an miRNA sequence arising from A. thaliana;

SEQ ID NO: 278 is UGACAGAAGAGAGUGAGCACA, an miRNA sequence arising from A. thaliana;

SEQ ID NO: 279 is UGACAGAAGAGAGUGAGCACA, an miRNA sequence arising from A. thaliana;

SEQ ID NO: 280 is UGACAGAAGAGAGUGAGCACA, an miRNA sequence arising from A. thaliana;

SEQ ID NO: 281 is UGACAGAAGAGAGUGAGCACA, an miRNA sequence arising from A. thaliana;

SEQ ID NO: 282 is UGACAGAAGAGAGUGAGCACA, an miRNA sequence arising from A. thaliana;

SEQ ID NO: 283 is UGACAGAAGAGAGUGAGCACA, an miRNA sequence arising from A. thaliana;

SEQ ID NO: 284 is UUGACAGAAGAAAGAGAGCAC, an miRNA sequence arising from A. thaliana;

SEQ ID NO: 285 is CGACAGAAGAGAGUGAGCACA, an miRNA sequence arising from A. thaliana;

SEQ ID NO: 286 is UUGACAGAAGAUAGAGAGCAC, an miRNA sequence arising from A. thaliana;

SEQ ID NO: 287 is UUGACAGAAGAUAGAGAGCAC, an miRNA sequence arising from A. thaliana;

SEQ ID NO: 288 is UUGACAGAAGAUAGAGAGCAC, an miRNA sequence arising from A. thaliana;

SEQ ID NO: 289 is CUGACAGAAGAUAGAGAGCAC, an miRNA sequence arising from A. thaliana;

SEQ ID NO: 290 is UCCCAAAUGUAGACAAAGCA, an miRNA sequence arising from A. thaliana;

SEQ ID NO: 291 is CCCCAAAUGUAGACAAAGCA, an miRNA sequence arising from A. thaliana;

SEQ ID NO: 292 is UUUGGAUUGAAGGGAGCUCUA, an miRNA sequence arising from A. thaliana;

SEQ ID NO: 293 is UUUGGAUUGAAGGGAGCUCUU, an miRNA sequence arising from A. thaliana;

SEQ ID NO: 294 is UUUGGAUUGAAGGGAGCUCCU, an miRNA sequence arising from A. thaliana;

SEQ ID NO: 295 is UUGGACUGAAGGGAGCUCCC, an miRNA sequence arising from A. thaliana;

SEQ ID NO: 296 is UUGGACUGAAGGGAGCUCCC, an miRNA sequence arising from A. thaliana;

SEQ ID NO: 297 is UUGGACUGAAGGGAGCUCCU, an miRNA sequence arising from A. thaliana;

SEQ ID NO: 298 is UGCCUGGCUCCCUGUAUGCCA, an miRNA sequence arising from A. thaliana;

SEQ ID NO: 299 is UGCCUGGCUCCCUGUAUGCCA, an miRNA sequence arising from A. thaliana;

SEQ ID NO: 300 is UGCCUGGCUCCCUGUAUGCCA, an miRNA sequence arising from A. thaliana;

SEQ ID NO: 301 is UUGAAAGUGACUACAUCGGGG, an miRNA sequence arising from A. thaliana;

SEQ ID NO: 302 is UCGAUAAACCUCUGCAUCCAG, an miRNA sequence arising from A. thaliana;

SEQ ID NO: 303 is UCGAUAAACCUCUGCAUCCAG, an miRNA sequence arising from A. thaliana;

SEQ ID NO: 304 is UUGAAGAGGACUUGGAACUUCGAU, an miRNA sequence arising from A. thaliana;

SEQ ID NO: 305 is UGGAGAAGCAGGGCACGUGCA, an miRNA sequence arising from A. thaliana;

SEQ ID NO: 306 is UGGAGAAGCAGGGCACGUGCA, an miRNA sequence arising from A. thaliana;

SEQ ID NO: 307 is UGGAGAAGCAGGGCACGUGCG, an miRNA sequence arising from A. thaliana;

SEQ ID NO: 308 is UCGGACCAGGCUUCAUCCCCC, an miRNA sequence arising from A. thaliana;

SEQ ID NO: 309 is UCGGACCAGGCUUCAUCCCCC, an miRNA sequence arising from A. thaliana;

SEQ ID NO: 310 is UCGGACCAGGCUUCAUUCCCC, an miRNA sequence arising from A. thaliana;

SEQ ID NO: 311 is UCGGACCAGGCUUCAUUCCCC, an miRNA sequence arising from A. thaliana;

SEQ ID NO: 312 is UCGGACCAGGCUUCAUUCCCC, an miRNA sequence arising from A. thaliana;

SEQ ID NO: 313 is UCGGACCAGGCUUCAUUCCCC, an miRNA sequence arising from A. thaliana;

SEQ ID NO: 314 is UCGGACCAGGCUUCAUUCCCC, an miRNA sequence arising from A. thaliana;

SEQ ID NO: 315 is UCGGACCAGGCU-UCAUUCCCC, an miRNA sequence arising from A. thaliana;

SEQ ID NO: 316 is UCGGACCAGGCUUCAUUCCCC, an miRNA sequence arising from A. thaliana;

SEQ ID NO: 317 is UGAAGCUGCCAGCAUGAUCUA, an miRNA sequence arising from A. thaliana;

SEQ ID NO: 318 is UGAAGCUGCCAGCAUGAUCUA, an miRNA sequence arising from A. thaliana;

SEQ ID NO: 319 is UUAAGCUGCCAGCAUGAUCUU, an miRNA sequence arising from A. thaliana;

SEQ ID NO: 320 is UGAAGCUGCCAGCAUGAUCUG, an miRNA sequence arising from A. thaliana;

SEQ ID NO: 321 is UCGCUUGGUGCAGGUCGGGAA, an miRNA sequence arising from A. thaliana;

SEQ ID NO: 322 is UCGCUUGGUGCAGGUCGGGAA, an miRNA sequence arising from A. thaliana;

SEQ ID NO: 323 is CAGCCAAGGAUGACUUGCCGA, an miRNA sequence arising from A. thaliana;

SEQ ID NO: 324 is CAGCCAAGGAUGACUUGCCGG, an miRNA sequence arising from A. thaliana;

SEQ ID NO: 325 is CAGCCAAGGAUGACUUGCCGG, an miRNA sequence arising from A. thaliana;

SEQ ID NO: 326 is GAGCCAAGGAUGACUUGCCGA, an miRNA sequence arising from A. thaliana;

SEQ ID NO: 327 is GAGCCAAGGAUGACUUGCCGG, an miRNA sequence arising from A. thaliana;

SEQ ID NO: 328 is GAGCCAAGGAUGACUUGCCGA, an miRNA sequence arising from A. thaliana;

SEQ ID NO: 329 is GAGCCAAGGAUGACUUGCCGG, an miRNA sequence arising from A. thaliana;

SEQ ID NO: 330 is UAGCCAAGGAUGACUUGCCUG, an miRNA sequence arising from A. thaliana;

SEQ ID NO: 331 is UAGCCAAGGAUGACUUGCCUG, an miRNA sequence arising from A. thaliana;

SEQ ID NO: 332 is UAGCCAAGGAUGACUUGCCUG, an miRNA sequence arising from A. thaliana;

SEQ ID NO: 333 is UAGCCAAGGAUGACUUGCCUG, an miRNA sequence arising from A. thaliana;

SEQ ID NO: 334 is UAGCCAAGGAUGACUUGCCUG, an miRNA sequence arising from A. thaliana;

SEQ ID NO: 335 is UAGCCAAGGAUGACUUGCCUG, an miRNA sequence arising from A. thaliana;

SEQ ID NO: 336 is UAGCCAAGGAUGACUUGCCUG, an miRNA sequence arising from A. thaliana;

SEQ ID NO: 337 is UGAUUGAGCCGUGUCAAUAUC, an miRNA sequence arising from A. thaliana;

SEQ ID NO: 338 is UGAUUGAGCCGCGCCAAUAUC, an miRNA sequence arising from A. thaliana;

SEQ ID NO: 339 is CGAUUGAGCCGUGCCAAUAUC, an miRNA sequence arising from A. thaliana;

SEQ ID NO: 340 is UGAUUGAGCCGUGCCAAUAUC, an miRNA sequence arising from A. thaliana;

SEQ ID NO: 341 is AGAAUCUUGAUGAUGCUGCAU, an miRNA sequence arising from A. thaliana;

SEQ ID NO: 342 is AGAAUCUUGAUGAUGCUGCAU, an miRNA sequence arising from A. thaliana;

SEQ ID NO: 343 is AGAAUCUUGAUGAUGCUGCAG, an miRNA sequence arising from A. thaliana;

SEQ ID NO: 344 is AGAAUCUUGAUGAUGCUGCAG, an miRNA sequence arising from A. thaliana;

SEQ ID NO: 345 is GGAAUCUUGAUGAUGCUGCAU, an miRNA sequence arising from A. thaliana;

SEQ ID NO: 346 is UUCGCUUGCAGAGAGAAAUCAC, an miRNA sequence arising from A. thaliana;

SEQ ID NO: 347 is UCCAAAGGGAUCGCAUUGAUC, an miRNA sequence arising from A. thaliana;

SEQ ID NO: 348 is UCCAAAGGGAUCGCAUUGAUC, an miRNA sequence arising from A. thaliana;

SEQ ID NO: 349 is UUGGCAUUCUGUCCACCUCC, an miRNA sequence arising from A. thaliana;

SEQ ID NO: 350 is UUGGCAUUCUGUCCACCUCC, an miRNA sequence arising from A. thaliana;

SEQ ID NO: 351 is CUGAAGUGUUUGGGGGAACUC, an miRNA sequence arising from A. thaliana;

SEQ ID NO: 352 is CUGAAGUGUUUGGGGGGACUC, an miRNA sequence arising from A. thaliana;

SEQ ID NO: 353 is CUGAAGUGUUUGGGGGGACUC, an miRNA sequence arising from A. thaliana;

SEQ ID NO: 354 is CUGAAGUGUUUGGGGGAACUC, an miRNA sequence arising from A. thaliana;

SEQ ID NO: 355 is CUGAAGUGUUUGGGGGAACUC, an miRNA sequence arising from A. thaliana;

SEQ ID NO: 356 is CUGAAGUGUUUGGGGGGACUC, an miRNA sequence arising from A. thaliana;

SEQ ID NO: 357 is UUCCACAGCUUUCUUGAACUG, an miRNA sequence arising from A. thaliana;

SEQ ID NO: 358 is UUCCACAGCUUUCUUGAACUU, an miRNA sequence arising from A. thaliana;

SEQ ID NO: 359 is UCAUUGAGUGCAGCGUUGAUG, an miRNA sequence arising from A. thaliana;

SEQ ID NO: 360 is UCAUUGAGUGCAUCGUUGAUG, an miRNA sequence arising from A. thaliana;

SEQ ID NO: 361 is UGUGUUCUCAGGUCACCCCUU, an miRNA sequence arising from A. thaliana;

SEQ ID NO: 362 is UGUGUUCUCAGGUCACCCCUG, an miRNA sequence arising from A. thaliana;

SEQ ID NO: 363 is UGUGUUCUCAGGUCACCCCUG, an miRNA sequence arising from A. thaliana;

SEQ ID NO: 364 is UGCCAAAGGAGAUUUGCCCUG, an miRNA sequence arising from A. thaliana;

SEQ ID NO: 365 is UGCCAAAGGAGAGUUGCCCUG, an miRNA sequence arising from A. thaliana;

SEQ ID NO: 366 is UGCCAAAGGAGAGUUGCCCUG, an miRNA sequence arising from A. thaliana;

SEQ ID NO: 367 is UGCCAAAGGAGAUUUGCCCCG, an miRNA sequence arising from A. thaliana;

SEQ ID NO: 368 is UGCCAAAGGAGAUUUGCCUCG, an miRNA sequence arising from A. thaliana;

SEQ ID NO: 369 is LJGCCAAAGGAGAUUUGCCCGG, an miRNA sequence arising from A. thaliana;

SEQ ID NO: 370 is AACUGACAGAAGAGAGUGAGCACACAA, a sequence expected to encode miRNA an arising from A. thaliana;

SEQ ID NO: 371 is AACUGACAGAAGAGAGUGAGCACAUGC, a sequence expected to encode miRNA an arising from A. thaliana;

SEQ ID NO: 372 is AACUGACAGAAGAGAGUGAGCACACAA, a sequence expected to encode miRNA an arising from A. thaliana;

SEQ ID NO: 373 is UGACAGAAGAGAGUGAGCACACAAAGGG, a sequence expected to encode miRNA an arising from A. thaliana;

SEQ ID NO: 374 is GGUGACAGAAGAGAGUGAGCACACAUGGUGG, a sequence expected to encode miRNA an arising from A. thaliana;

SEQ ID NO: 375 is UGGUGACAGAAGAGAGUGAGCACACAUGGUGG, a sequence expected to encode miRNA an arising from A. thaliana;

SEQ ID NO: 376 is UUGACAGAAGAAAGAGAGCAC, a sequence expected to encode miRNA an arising from A. thaliana;

SEQ ID NO: 377 is GGCGACAGAAGAGAGUGAGCACACAUGGCU, a sequence expected to encode miRNA an arising from A. thaliana;

SEQ ID NO: 378 is GUUGACAGAAGAUAGAGAGCACA, a sequence expected to encode miRNA an arising from A. thaliana;

SEQ ID NO: 379 is UGUUGACAGAAGAUAGAGAGCACA, a sequence expected to encode miRNA an arising from A. thaliana;

SEQ ID NO: 380 is UUGUUGACAGAAGAUAGAGAGCAC, a sequence expected to encode miRNA an arising from A. thaliana;

SEQ ID NO: 381 is UGACAGAAGAUAGAGAGCAC, a sequence expected to encode miRNA an arising from A. thaliana;

SEQ ID NO: 382 is CGUUUGGAUUGAAGGGAGCUCCUU, a sequence expected to encode miRNA an arising from A. thaliana;

SEQ ID NO: 383 is UUGAUUGGACUGAAGGGAGCUCCC, a sequence expected to encode miRNA an arising from A. thaliana;

SEQ ID NO: 384 is CUAUGCUUGGACUGAAGGGAGCUCCC, a sequence expected to encode miRNA an arising from A. thaliana;

SEQ ID NO: 385 is GCCUGGCUCCCUGUAUGCCAUAU, a sequence expected to encode miRNA an arising from A. thaliana;

SEQ ID NO: 386 is GUCGUGCCUGGCUCCCUGUAUGCCACAAG, a sequence expected to encode miRNA an arising from A. thaliana;

SEQ ID NO: 387 is CGUUAUGCCUGGCUCCCUGUAUGCCACGAG, a sequence expected to encode miRNA an arising from A. thaliana;

SEQ ID NO: 388 is UGAAUAGAUCGAUAAACCUCUGCAUCCAGC, a sequence expected to encode miRNA an arising from A. thaliana;

SEQ ID NO: 389 is GAAUCGAUCGAUAAACCUCUGCAUCCAGC, a sequence expected to encode miRNA an arising from A. thaliana;

SEQ ID NO: 390 is CCAUGUUGGAGAAGCAGGGCACGUGCAAAC, a sequence expected to encode miRNA an arising from A. thaliana;

SEQ ID NO:.391 is AAGAUGGAGAAGCAGGGCACGUGCA, a sequence expected to encode miRNA an arising from A. thaliana;

SEQ ID NO: 392 is CUUGAUGGAGAAGCAGGGCACGUGCGA, a sequence expected to encode miRNA an arising from A. thaliana;

SEQ ID NO: 393 is GUAUCCUCGGACCAGGCUUCAUCCCCC, a sequence expected to encode miRNA an arising from A. thaliana;

SEQ ID NO: 394 is UCGGACCAGGCUUCAUCCCCC, a sequence expected to encode miRNA an arising from A. thaliana;

SEQ ID NO: 395 is UCGGACCAGGCUUCAUUCCCC, a sequence expected to encode miRNA an arising from A. thaliana;

SEQ ID NO: 396 is UCGGACCAGGCUUCAUUCCCC, a sequence expected to encode miRNA an arising from A. thaliana;

SEQ ID NO: 397 is UCGGACCAGGCUUCAUUCCCC, a sequence expected to encode miRNA an arising from A. thaliana;

SEQ ID NO: 398 is UCGGACCAGGCUUCAUUCCCCUCAA, a sequence expected to encode miRNA an arising from A. thaliana;

SEQ ID NO: 399 is UCGGACCAGGCUUCAUUCCCCUCAACU, a sequence expected to encode miRNA an arising from A. thaliana;

SEQ ID NO: 400 is UCGGACCAGGCUUCAUUCCCCUCAAC, a sequence expected to encode miRNA an arising from A. thaliana;

SEQ ID NO: 401 is CUGAUGAAGCUGCCAGCAUGAUCUAAUUA, a sequence expected to encode miRNA an arising from A. thaliana;

SEQ ID NO: 402 is AAGUGAAGCUGCCAGCAUGAUCUAUCUUUG, a sequence expected to encode miRNA an arising from A. thaliana;

SEQ ID NO: 403 is CAGUUAAGCUGCCAGCAUGAUCUUGUC, a sequence expected to encode miRNA an arising from A. thaliana;

SEQ ID NO: 404 is CUCGGAUUCGCUUGGUGCAGGUCGGGAACC, a sequence expected to encode miRNA an arising from A. thaliana;

SEQ ID NO: 405 is CUCGGAUUCGCUUGGUGCAGGUCGGGAAC, a sequence expected to encode miRNA an arising from A. thaliana;

SEQ ID NO: 406 is AGUGUGCAGCCAAGGAUGACUUGCCGAUU, a sequence expected to encode miRNA an arising from A. thaliana;

SEQ ID NO: 407 is AUAAUGCAGCCAAGGAUGACUUGCCGGAAC, a sequence expected to encode miRNA an arising from A. thaliana;

SEQ ID NO: 408 is GUUCAGCCAAGGAUGACUUGCCGGUA, a sequence expected to encode miRNA an arising from A. thaliana;

SEQ ID NO: 409 is GAUUGAGCCAAGGAUGACUUGCCGAU, a sequence expected to encode miRNA an arising from A. thaliana;

SEQ ID NO: 410 is UUGAGCCAAGGAUGACUUGCCGGUU, a sequence expected to encode miRNA an arising from A. thaliana;

SEQ ID NO: 411 is GAUUGAGCCAAGGAUGACUUGCCGAU, a sequence expected to encode miRNA an arising from A. thaliana;

SEQ ID NO: 412 is GGUUGAGCCAAGGAUGACUUGCCGGGUU, a sequence expected to encode miRNA an arising from A. thaliana;

SEQ ID NO: 413 is GUGUGGUAGCCAAGGAUGACUU, a sequence expected to encode miRNA an arising from A. thaliana;

SEQ ID NO: 414 is AUUUGGUAGCCAAGGAUGACUU, a sequence expected to encode miRNA an arising from A. thaliana;

SEQ ID NO: 415 is UUUAGUAGCCAAGGAUGACUU, a sequence expected to encode miRNA an arising from A. thaliana;

SEQ ID NO: 416 is AUUUGGUAGCCAAGGAUGACUU, a sequence expected to encode miRNA an arising from A. thaliana;

SEQ ID NO: 417 is UUAAUAGCCAAGGAUGACUU, a sequence expected to encode miRNA an arising from A. thaliana;

SEQ ID NO: 418 is AUUUGGUAGCCAAGGAUGACUU, a sequence expected to encode miRNA an arising from A. thaliana;

SEQ ID NO: 419 is UUUAGUAGCCAAGGAUGACUU, a sequence expected to encode miRNA an arising from A. thaliana;

SEQ ID NO: 420 is UGAUUGAGCCGUGUCAAUAUCUC, a sequence expected to encode miRNA an arising from A. thaliana;

SEQ ID NO: 421 is UUAUCUGAUUGAGCCGCGCCAAUAUCUCAGU, a sequence expected to encode miRNA an arising from A. thaliana;

SEQ ID NO: 422 is UGUUCGAUUGAGCCGUGCCAAUAUCACGCG, a sequence expected to encode miRNA an arising from A. thaliana;

SEQ ID NO: 423 is UUAUUUGAUUGAGCCGUGCCAAUAUCAC, a sequence expected to encode miRNA an arising from A. thaliana;

SEQ ID NO: 424 is AAUGAGAAUCUUGAUGAUGCUGCAUCGGCA, a sequence expected to encode miRNA an arising from A. thaliana;

SEQ ID NO: 425is UAUGAGAAUCUUGAUGAUGCUGCAUC, a sequence expected to encode miRNA an arising from A. thaliana;

SEQ ID NO: 426 is UAUGAGAAUCUUGAUGAUGCUGCAGCUGCAA, a sequence expected to encode miRNA an arising from A. thaliana;

SEQ ID NO: 427 is GUUUGAGAAUCUUGAUGAUGCUGCAGCGGCAA, a sequence expected to encode miRNA an arising from A. thaliana;

SEQ ID NO: 428 is GAAUCUUGAUGAUGCUGCAUC, a sequence expected to encode miRNA an arising from A. thaliana;

SEQ ID NO: 429 is GAGGAAGGAUCCAAAGGGAUCGCAUUGAUCCUAA, a sequence expected to encode miRNA an arising from A. thaliana;

SEQ ID NO: 430 is GAAAGGAUCCAAAGGGAUCGCAUUGAUCCU, a sequence expected to encode miRNA an arising from A. thaliana;

SEQ ID NO: 431 is AUCUUUGGCAUUCUGUCCACCUCCUUC, a sequence expected to encode miRNA an arising from A. thaliana;

SEQ ID NO: 432 is CAGAGAUCUUUGGCAUUCUGUCCACCUCCUCU, a sequence expected to encode miRNA an arising from A. thaliana;

SEQ ID NO: 433 is CACUGAAGUGUUUGGGGGAACUCCCGGA, a sequence expected to encode miRNA an arising from A. thaliana;

SEQ ID NO: 434 is ACUGAAGUGUUUGGGGGGACUCUUG, a sequence expected to encode miRNA an arising from A. thaliana;

SEQ ID NO: 435 is ACUGAAGUGUUUGGGGGGACUCUU, a sequence expected to encode miRNA an arising from A. thaliana;

SEQ ID NO: 436 is CACUGAAGUGUUUGGGGGAACUCCCGA, a sequence expected to encode miRNA an arising from A. thaliana;

SEQ ID NO: 437 is CUACUGAAGUGUUUGGGGGAACUCCC, a sequence expected to encode miRNA an arising from A. thaliana;

SEQ ID NO: 438 is ACUGAAGUGUUUGGGGGGACUCUAGGUGACA, a sequence expected to encode miRNA an arising from A. thaliana;

SEQ ID NO: 439 is UUCCACAGCUUUCUUGAACU, a sequence expected to encode miRNA an arising from A. thaliana;

SEQ ID NO: 440 is CAUACUUUUCCACAGCUUUCUUGAACUUUC, a sequence expected to encode miRNA an arising from A. thaliana;

SEQ ID NO: 441 is ACAUCAUUGAGUGCAUCGUUGAUGUA, a sequence expected to encode miRNA an arising from A. thaliana;

SEQ ID NO: 442 is UUGUGUUCUCAGGUCACCCCUUUGAA, a sequence expected to encode miRNA an arising from A. thaliana;

SEQ ID NO: 443 is CAUGUGUUCUCAGGUCACCCCUGCUG, a sequence expected to encode miRNA an arising from A. thaliana;

SEQ ID NO: 444 is AUGUGUUCUCAGGUCACCCCUGCUG, a sequence expected to encode miRNA an arising from A. thaliana;

SEQ ID NO: 445 is AUCUGCCAAAGGAGAUUUGCCCUGU, a sequence expected to encode miRNA an arising from A. thaliana;

SEQ ID NO: 446 is ACCUGCCAAAGGAGAGUUGCCCUGAAACUGGU, a sequence expected to encode miRNA an arising from A. thaliana;

SEQ ID NO: 447 is CUUGCCAAAGGAGAGUUGCCCUGUCA, a sequence expected to encode miRNA an arising from A. thaliana;

SEQ ID NO: 448 is CUCUGCCAAAGGAGAUUUGCCCCGCAAUUCA, a sequence expected to encode miRNA an arising from A. thaliana;

SEQ ID NO: 449 is UCCUCUGCCAAAGGAGAUUUGCCUCGC, a sequence expected to encode miRNA an arising from A. thaliana;

SEQ ID NO: 450 is UGAGCUCUCUGCCAAAGGAGAUUUGCCCGGUAA, a sequence expected to encode miRNA an arising from A. thaliana;

SEQ ID NO: 451 is GCAAAGAAACUGACAGAAGAGAGUGAGCACACAAAGGCAAUUUGCAUAUCAUUG CACUUGCUUCUCUUGCGUGCUCACUGCUCUUUCUGUCAGAUUCCGGUG, an RNA fragment encoding a hairpin motif, the fragment arising from A. thaliana;

SEQ ID NO: 452 is CAGAGAAAACUGACAGAAGAGAGUGAGCACAUGCAGGCACUGUUAUGUGUCUAU AACUUUGCGUGUGCGUGCUCACCUCUCUUUCUGUCAGUUGCCUAUC, an RNA fragment encoding a hairpin motif, the fragment arising from A. thaliana;

SEQ ID NO: 453 is GCAUAGAAACUGACAGAAGAGAGUGAGCACACAAAGGCACUUUGCAUGUUCGAU GCAUUUGCUUCUCUUGCGUGCUCACUGCUCUAUCUGUCAGAUUCCGGCU, an RNA fragment encoding a hairpin motif, the fragment arising from A. thaliana;

SEQ ID NO: 454 is GAAAAGAAGUUGACAGAAGAGAGUGAGCACACAAAGGGGAAGUUUGUAUAAAAGU UUUGUAUAUGGUUGCUUUUGCGUGCUCACUCUCUUUUUGUCAUAACUUCUCC, an RNA fragment encoding a hairpin motif, the fragment arising from A. thaliana;

SEQ ID NO: 455 is AAUUAGGAGGUGACAGAAGAGAGUGAGCACACAUGGUGGUUUCUUGCAUGCUUU UUUGAUUAGGGUUUCAUGCUUGAAGCUAUGUGUGCUUACUCUCUCUCUGUCACC CCUUCUCU, an RNA fragment encoding a hairpin motif, the fragment arising from A. thaliana;

SEQ ID NO: 456 is GAAUUGAUGGUGACAGAAGAGAGUGAGCACACAUGGUGGCUUUCUUGCAUAUUU GAAGGUUCCAUGCUUGAAGCUAUGUGUGCUCACUCUCUAUCCGUCACCCCCUUCU C, an RNA fragment encoding a hairpin motif, the fragment arising from A. thaliana;

SEQ ID NO: 457 is AUGAAAAAUGUUGACAGAAGAAAGAGAGCACAACCUGGGAUUAGCAAAAAGAUA GUUUUGCCCUUGUCGGGAGUGUGCUCUCUUUCCUUCUGCCACCAUCAUUGCG, an RNA fragment encoding a hairpin motif, the fragment arising from A. thaliana;

SEQ ID NO: 458 is AUAACGAAGGCGACAGAAGAGAGUGAGCACACAUGGCUCUUUUUCUAGCAUGCU CAUGCUCGAAAGCUCUGCGUGCUUACUCUCUUCUUGUCUCCUGCUCUCU, an RNA fragment encoding a hairpin motif, the fragment arising from A. thaliana;

SEQ ID NO: 459 is AUUGAUAGUGUUGACAGAAGAUAGAGAGCACAGAUGAUGAGAUACAAUUCGGAG CAUGUUCUUUGCAUCUUACUCCUUUGUGCUCUCUAGCCUUCUGUCAUCACCUUUU AU, an RNA fragment encoding a hairpin motif, the fragment arising from A. thaliana;

SEQ ID NO: 460 is AUUGAUAGUGUUGACAGAAGAUAGAGAGCACAGAUGAUAAGAUACAAUUCCUCG CAGCUUCUUUGCAUCUUACUCCUUUGUGCUCUCUAGCCUUCUGUCAUCACCCGUU AU, an RNA fragment encoding a hairpin motif, the fragment arising from A. thaliana;

SEQ ID NO: 461 is AUGUUGGUUGUUGACAGAAGAUAGAGAGCACUAAGGAUGACAUGCAAGUACAUA CAUAUAUAUCAUCACACCGCAUGUGGAUGAUAAAAUAUGUAUAACAAAUUCAAA GAAAGAGAGGGAGAGAAAGAGAGAGAACCUGCAUCUCUACUCUUUUGUGCUCUC UAUACUUCUGUCACCACCUUUAU, an RNA fragment encoding a hairpin motif, the fragment arising from A. thaliana;

SEQ ID NO: 462 is AGUGUGGUUGCUGACAGAAGAUAGAGAGCACUAAGGAUGCUAUGCAAAACAGAC ACAGAUAUGUGUUUCUAAUUGUAUUUCAUACUUUAACCUCAAAGUUGAUAUAAA AAAAGAAAGAAAGAUAGAAGAGCUAGAAGACUAUCUGCAUCUCUAUUCCUAUGU GCUCUCUAUGCUUCUGUCAUCACCUUUCUU, an RNA fragment encoding a hairpin motif, the fragment arising from A. thaliana;

SEQ ID NO: 463 is AUCUCUGUGCUUCUUUGUCUACAAUUUUGGAAAAAGUGAUGACGCCAUUGCUCU UUCCCAAAUGUAGACAAAGCAAUACCGUGAU, an RNA fragment encoding a hairpin motif, the fragment arising from A. thaliana;

SEQ ID NO: 464 is AUCUCUGUGCUUCUUUGUCUACACUUUUGGAAAAGGUGAUGAUAUCAUUGCUUU UCCCCAAAUGUAGACAAAGCAAUACCGUGAU, an RNA fragment encoding a hairpin motif, the fragment arising from A. thaliana;

SEQ ID NO: 465 is ACGAUGGAAGUAGAGCUCCUUAAAGUUCAAACAUGAGUUGAGCAGGGUAAAGAA AAGCUGCUAAGCUAUGGAUCCCAUAAGCCCUAAUCCUUGUAAAGUAAAAAAGGA UUUGGUUAUAUGGAUUGCAUAUCUCAGGAGCUUUAACUUGCCCUUUAAUGGCUU UUACUCUUCUUUGGAUUGAAGGGAGCUCUACAUCUUCUUU, an RNA fragment encoding a hairpin motif, the fragment arising from A. thaliana;

SEQ ID NO: 466 is GAAGAAGAGGAAGAGCUCCUUGAAGUUCAAUGGAGGGUUUAGCAGGGUGAAGUA AAGCUGCUAAGCUAUGGAUCCCAUAAGCCUUAUCAAAUUCAAUAUAAUUGAUGA UAAGGUUUUUUUUAUGGAUGCCAUAUCUCAGGAGCUUUCACUUACCCCUUUAAU GGCUUCACUCUUCUUUGGAUUGAAGGGAGCUCUUCAUCUCUCCA, an RNA fragment encoding a hairpin motif, the fragment arising from A. thaliana;

SEQ ID NO: 467 is GUGUAACAGAAGGAGCUCCCUUCCUCCAAAACGAAGAGGACAAGAUUUGAGGAA CUAAAAUGCAGAAUCUAAGAGUUCAUGUCUUCCUCAUAGAGAGUGCGCGGUGUU AAAAGCUUGAAGAAAGCACACUUUAAGGGGAUUGCACGACCUCUUAGAUUCUCC CUCUUUCUCUACAUAUCAUUCUCUUCUCUUCGUUUGGAUUGAAGGGAGCUCCUU UUCUUCUUC, an RNA fragment encoding a hairpin motif, the fragment arising from A. thaliana;

SEQ ID NO: 468 is UAUAUGUAGAGAGAGCUUCCUUGAGUCCAUUCACAGGUCGUGAUAUGAUUCAAU UAGCUUCCGACUCAUUCAUCCAAAUACCGAGUCGCCAAAAUUCAAACUAGACUCG UUAAAUGAAUGAAUGAUGCGGUAGACAAAUUGGAUCAUUGAUUCUCUUUGAUUG GACUGAAGGGAGCUCCCUCUCUCUUUU, an RNA fragment encoding a hairpin motif, the fragment arising from A. thaliana;

SEQ ID NO: 469 is GGUGGAGGAAGAGAGCUUUCUUCGGUCCACUCAUGGAGUAAUAUGUGAGAUUUA AUUGACUCUCGACUCAUUCAUCCAAAUACCAAAUGAAAGAAUUUGUUCUCAUAU GGUAAAUGAAUGAAUGAUGCGAGAGACAAAUUGAGUCUUCACUUCUCUAUGCUU GGACUGAAGGGAGCUCCCUAUUUUUAUC, an RNA fragment encoding a hairpin motif, the fragment arising from A. thaliana;

SEQ ID NO: 470 is UAGAUAUAGAAGGAGAUUCUUUCAGUCCAGUCAUGGAUAGAAAAAGAAGAGGGU AGAAAUAUCUGCCGACUCAUCCAUCCAAACACUCGUGGUAGAGAAACGAUAAAU UUAAACCGCAGUGACUGUGUGAAUGAUGCGGGAGAUAUUUUUGAUCCUUCUUUA UCUGUGUUUGGACUGAAGGGAGCUCCUUCUUUUUCUA, an RNA fragment encoding a hairpin motif, the fragment arising from A. thaliana;

SEQID NO: 471is UAUAUAUGUAUGCCUGGCUCCCUGUAUGCCAUAUGCUGAGCCCAUCGAGUAUCG AUGACCUCCGUGGAUGGCGUAUGAGGAGCCAUGCAUAUCCUCAUA, an RNA fragment encoding a hairpin motif, the fragment arising from A. thaliana;

SEQ ID NO: 472 is AUAAUAGUCGUGCCUGGCUCCCUGUAUGCCACAAGAAAACAUCGAUUUAGUUUC AAAAUCGAUCACUAGUGGCGUACAGAGUAGUCAAGCAUGACCAAAGC, an RNA fragment encoding a hairpin motif, the fragment arising from A. thaliana;

SEQ ID NO: 473 is UUUGUCGUUAUGCCUGGCUCCCUGUAUGCCACGAGUGGAUACCGAUUUUGGUUU UAAAAUCGGCUGCCGGUGGCGUACAAGGAGUCAAGCAUGACCAGAAG, an RNA fragment encoding a hairpin motif, the fragment arising from A. thaliana;

SEQ ID NO: 474 is GAUCAAUGCAUUGAAAGUGACUACAUCGGGGUUCCGAUUUUUUUUGUUCUUCAU AUGAUGAAGCGGAAACAGUAAUCAACCCUGGUUUAGUCACUUUCACUGCAUUAA UC, an RNA fragment encoding a hairpin motif, the fragment arising from A. thaliana;

SEQ ID NO: 475 is GUGAGAGUCGCUGGAGGCAGCGGUUCAUCGAUCUCUUCCUGUGAACACAUUAAA AAUGUAAAAGCAUGAAUAGAUCGAUAAACCUCUGCAUCCAGCGUUUGCCUC, an RNA fragment encoding a hairpin motif, the fragment arising from A. thaliana;

SEQ ID NO: 476 is AGUGAAGUCGCUGGAGGCAGCGGUUCAUCGAUCAAUUCCUGUGAAUAUUUAUUU UUGUUUACAAAAGCAAGAAUCGAUCGAUAAACCUCUGCAUCCAGCGCUGCUUGC, an RNA fragment encoding a hairpin motif, the fragment arising from A. thaliana;

SEQ ID NO:477is GGUGGAUAAAAUCGAGUUCCAACCUCUUCAACGACAACGAUUUCAACACUCUCU UCCAGGAACAACUUCCUCCAGGCAGAUGAUACUAAAGUGCUGGAGUUCCCGGUU CCUGAGAGUGAGUCCAUAUCAAAAUGCGCAUUCGUUAUCACUUGGUUGAACCCA UUUGGGGAUUUAAAUUUGGAGGUGAAAUGGAACGCGUAAUUGAUGACUCCUACG UGGAACCUCUUCUUAGGAAGAGCACGGUCGAAGAAGUAACUGCGCAGUGCUUAA AUCGUAGAUGCUAAAGUCGUUGAAGAGGACUUGGAACUUCGAUAUUAUCCCCC, an RNA fragment encoding a hairpin motif, the fragment arising from A. thaliana;

SEQ ID NO: 478 is AUCUCCAUGUUGGAGAAGCAGGGCACGUGCAAACCAACAAACACGAAAUCCGUC UCAUUUGCUUAUUUGCACGUACUUAACUUCUCCAACAUGAGCUC, an RNA fragment encoding a hairpin motif, the fragment arising from A. thaliana;

SEQ ID NO: 479 is AUGAGCAAGAUGGAGAAGCAGGGCACGUGCAUUACUAGCUCAUAUAUACACUCU CACCACAAAUGCGUGUAUAUAUGCGGAAUUUUGUGAUAUAGAUGUGUGUGUGUG UUGAGUGUGAUGAUAUGGAUGAGUUAGUUCUUCAUGUGCCCAUCUUCACCAUCA UGACCAC, an RNA fragment encoding a hairpin motif, the fragment arising from A. thaliana;

SEQ ID NO: 480 is UAACACUUGAUGGAGAAGCAGGGCACGUGCGAACACAAAUGAAAUCGAUCGGUA CUUGUUGAUCAUAUUUUCGCACGUGUUCUACUACUCCAACACGUGUCU, an RNA fragment encoding a hairpin motif, the fragment arising from A. thaliana;

SEQ ID NO: 481 is UUUCAGUUGAGGGGAAUGUUGUCUGGAUCGAGGAUAUUAUAGAUAUAUACAUGU GUAUGUUAAUGAUUCAAGUGAUCAUAGAGAGUAUCCUCGGACCAGGCUUCAUCC CCCCCAACAUGUU, an RNA fragment encoding a hairpin motif, the fragment arising from A. thaliana;

SEQ ID NO: 482 is UUUCUGUUGUGGGGAAUGUUGUUUGGAUCGAGGAUAUCAUAAACGCAUACACAU GUUUAUAUGUUAUGAUGCAUUAUAUGACUGAUGUAAUGUACAUAUAUAUACAUA CAUGCCACAUGGUAUCGUCGGACCAGGCUUCAUCCCCCUCAACAUGUU, an RNA fragment encoding a hairpin motif, the fragment arising from A. thaliana;

SEQ ID NO: 483 is UCUCUUUUGAGGGGACUGUUGUCUGGCUCGAGGACUCUGGCUCGCUCUAUUCAU GUUGGAUCUCUUUCGAUCUAACAAUCGAAUUGAACCUUCAGAUUUCAGAUUUGA UUAGGGUUUUAGCGUCUUCGGACCAGGCUUCAUUCCCCCCAAUUGUUG, an RNA fragment encoding a hairpin motif, the fragment arising from A. thaliana;

SEQ ID NO: 484 is UUUCUUUUGAGGGGACUGUUGUCUGGCUCGAGGACUCUUAUUCUAAUACAAUCU CAUUUGAAUACAUUCAGAUCUGAUGAUUGAUUAGGGUUUUAGUGUCGUCGGACC AGGCUUCAUUCCCCCCAAUUAUCA, an RNA fragment encoding a hairpin motif, the fragment arising from A. thaliana;

SEQ ID NO: 485 is UUAGUGUUGAGAGGAUUGUUGUCUGGCUCGAGGUCAUGAAGAAGAGAAUCACUC GAAUUAAUUUGGAAGAACAAAUUAAGAAAACCCUAGAUGAUUCUCGGACCAGGC UUCAUUCCCCCUAACCUACU, an RNA fragment encoding a hairpin motif, the fragment arising from A. thaliana;

SEQ ID NO: 486 is UUAGGGUUGAGAGGAAUAUUGUCUGGCUCGAGGUCAUGAAGAAGAUCGGUAGAU UGAUUCAUUUUAAAGAGUGAAAUCCCUAAAUGAUUCUCGGACCAGGCUUCAUUC CCCCCAACCGACA, an RNA fragment encoding a hairpin motif, the fragment arising from A. thaliana;

SEQ ID NO: 487 is UUCCUUUUGAGGGGAAUGUUGUCUGGCACGAGGCCCUUAACUUAGAUCUAUAUU UGAUUAUAUAUAUAUGUCUCUUCUUUAUUCAUUAGUCUAUACAUGAAUGAUCAU UUUACGGUUAAUGACGUCGGACCAGGCUUCAUUCCCCUCAAUUAUAU, an RNA fragment encoding a hairpin motif, the fragment arising from A. thaliana;

SEQ ID NO: 488 is CAAAAGUUCAGGUGAAUGAUGCCUGGCUCGAGACCAUUCAAUCUCAUGAUCUCA UGAUUAUAACGAUGAUGAUGAUGAUGUCGGACCAGGCUUCAUUCCCCUCAACUU ACA, an RNA fragment encoding a hairpin motif, the fragment arising from A. thaliana;

SEQ ID NO: 489 is UUAGGGUUUAGAGGAAUGUUGUUUGGCUCGAGGUCAUGGAGAGUAAUUCGUUAA CCCAACUCAAAACUCUAAAUGAUUCUCGGACCAGGCUUCAUUCCCCUCAACCUAU U, an RNA fragment encoding a hairpin motif, the fragment arising from A. thaliana;

SEQ ID NO: 490 is CGGCAUCUGAUGAAGCUGCCAGCAUGAUCUAAUUAGCUUUCUUUAUCCUUUGUU GUGUUUCAUGACGAUGGUUAAGAGAUCAGUCUCGAUUAGAUCAUGUUCGCAGUU UCACCCGUUGACU, an RNA fragment encoding a hairpin motif, the fragment arising from A. thaliana;

SEQ ID NO: 491 is AGGGAACAAGUGAAGCUGCCAGCAUGAUCUAUCUUUGGUUAAGAGAUGAAUGUG GAAACAUAUUGCUUAAACCCAAGCUAGGUCAUGCUCUGACAGCCUCACUCCUUCC UG, an RNA fragment encoding a hairpin motif, the fragment arising from A. thaliana;

SEQ ID NO: 492 is CCAGUAGCAGUUAAGCUGCCAGCAUGAUCUUGUCUUCCUCUCUUAGGUUUCAUA UAUAGUUAAUAAAUAUUUUAUAUAUUUCUUGUUCUUACAAGAUUAUAUGAUCAU AGCUUAGAGAGAGAGAGAGACUAGGUCAUGCUGGUAGUUUCACCUGCUAAUG, an RNA fragment encoding a hairpin motif, the fragment arising from A. thaliana;

SEQ ID NO: 493 is UUUUAGAAGCUGAAGCUGCCAGCAUGAUCUGGUAAUCGCUACAUACGACAUACA CACAUCACUAAACUUCUUUAUAAUUUAUGCACACACAUACAGCUCUUAAUGGCC ACAACUCAAAGUUAUAAUUAGUGCAUGAUCUCUAGUUAUUUGACUGCUUUUAAU AUAUGUUUAUGGAUUCACGCAUGUGUGUGUAUGUACAUAAUUUACAUGCAUGCA CUUUGUGUAUGGUACACAUCAAUUUGAACCCGUUCAAAAUUCUGUUUUUAUUAG UAUAUAUAUAGAUGUAUGUGGUGUGUGUGUCAGUGUGUGUGUGUGUUUAUAGA UAGUAGUACUAGGUCAUCCUGCAGCUUCAGUCACUAAA, an RNA fragment encoding a hairpin motif, the fragment arising from A. thaliana;

SEQ ID NO: 494 is GGGCUCGGAUUCGCUUGGUGCAGGUCGGGAACCAAUUCGGCUGACACAGCCUCG UGACUUUUAAACCUUUAUUGGUUUGUGAGCAGGGAUUGGAUCCCGCCUUGCAUC AACUGAAUCGGAUCCU, an RNA fragment encoding a hairpin motif, the fragment arising from A. thaliana;

SEQ ID NO: 495 is GGUCUCGGAUUCGCUUGGUGCAGGUCGGGAACUGAUUGGCUGACACCGACACGU GUCUUGUCAUGGUUGGUUUGUGAGCUCCCGUCUUGUAUCAACUGAAUCGGAGUC C, an RNA fragment encoding a hairpin motif, the fragment arising from A. thaliana;

SEQ ID NO: 496 is AAGUAGUGUGCAGCCAAGGAUGACUUGCCGAUUUAAAUGAUCUUUCUUUAUACU CUAUUAAGACAAUUUAGUUUCAAACUUUUUUUUUUUUUUUUUUUUGAAGGAUUC AGGAAGAAAUUAGGAUAUAUUAUUCCGUAUAAAAUACAAGAUAUAUAAAACCAA AAAGAAAAAGUAACAUGAUCGGCAAGUUGUCCUUGGCUACACGUUACUU, an RNA fragment encoding a hairpin motif, the fragment arising from A. thaliana;

SEQ ID NO: 497 is GAGUAUAAUGCAGCCAAGGAUGACUUGCCGGAACGUUGUUAACCAUGCAUAUGA AUAAUGUGAUGAUUAAUUAUGUGAUGAACAUAUUUCUGGCAAGUUGUCCUUCGG CUACAUUUUGCUC, an RNA fragment encoding a hairpin motif, the fragment arising from A. thaliana;

SEQ ID NO: 498 is CAUUGUUGUUCAGCCAAGGAUGACUUGCCGGUAGCUUGUAUUAUGAUUACUCUA UAUUCGAUUUAUAUUAUGGAGAUGAUGGUUUAUAUAUAUUUACUUAUCUACAUA GUUUUAGUUGAUUUUUUUUCGUACGUAAUAUAAUACGAAAAAGUAUUUACUUAU UUAUAUAUGUGUGUUGGGGCAAGAAGUGUAACCAAGCUAGCCCGGCAAGUCAUC UAUGGCUAUGCAACUGUC, an RNA fragment encoding a hairpin motif, the fragment arising from A. thaliana;

SEQ ID NO: 499 is AAAUGAGAUUGAGCCAAGGAUGACUUGCCGAUUUUCUCAACGAAUCUUACUGAU UAUGGUAUCCGGCAAGUUGACUUUGGCUCUGUUUCCUUC, an RNA fragment encoding a hairpin motif, the fragment arising from A. thaliana;

SEQ ID NO: 500 is GAAUGGAAUUGAGCCAAGGAUGACUUGCCGGUUUAAACCCAACCGGUUUAUGAC CAUUGAUUUGGUCUCAUUCACAAUCUGUUGAUUCGUGUCUGGCAAGUUGACCUU GGCUCUGCUUCGUUC, an RNA fragment encoding a hairpin motif, the fragment arising from A. thaliana;

SEQ ID NO: 501 is GAAUGAGAUUGAGCCAAGGAUGACUUGCCGAUGUUAUCAACAAAUCUUAACUGA UUUUGGUGUCCGGCAAGUUGACCUUGGCUCUGUUUCCUUC, an RNA fragment encoding a hairpin motif, the fragment arising from A. thaliana;

SEQ ID NO: 502 is GAAUGAGGUUGAGCCAAGGAUGACUUGCCGGGUUUUUUUACCAAUGAAUCUAAU UAACUGAUUCUGGUGUCCGGCAAGUUGACCUUGGCUCUGUUUCCUUC, an RNA fragment encoding a hairpin motif, the fragment arising from A. thaliana;

SEQ ID NO: 503 is ACUUGUGUGGUAGCCAAGGAUGACUUGCCUGCGUUUUAGACCAUAUAUAUCAAA GACUCACUCGAUCGAUAGUCUUAGAGUUGGUUGGUCGUCAGGCAGUCUCCUUGG CUAUUCAAACAAU, an RNA fragment encoding a hairpin motif, the fragment arising from A. thaliana;

SEQ ID NO: 504 is UCAUAUUUGGUAGCCAAGGAUGACUUGCCUGACUCUUUGUGUAAAAUGUUUAGU GUCUUGUUUGAAGUCACUAUAAGUUGUAUCAAGCAAUGACCAUUUUGCUUAUAA AAAAGAUAUCAGGCAGUCUCCUUGGCUAUCCUUAUAUG, an RNA fragment encoding a hairpin motif, the fragment arising from A. thaliana;

SEQ ID NO: 505 is UCAUGUUUAGUAGCCAAGGAUGACUUGCCUGAUCUUUUUCACCUCCAUGAUUCA AUUUGUAAUUCAUGGGUUUUGGAUUAUUAUACAUUCAAAAGUAUAAUAAUUUG AAAUCAUGUUGAAUCUUGCGGGUUAGGUUUCAGGCAGUCUCCUUGGCUAUCUUG ACAUG, an RNA fragment encoding a hairpin motif, the fragment arising from A. thaliana;

SEQ ID NO: 506 is CAAUAUUUGGUAGCCAAGGAUGACUUGCCUGCUUCUCUGAACAAAAUGGUCGAU GUCAUGUUUUGAAGUGACUAUAAGUUAUACCAAGAAAUGACCAUUUUGUUUAUA AAUAGACAUCAGGCAGUCUCCUUGGCUAUCCUUAUAUG, an RNA fragment encoding a hairpin motif, the fragment arising from A. thaliana;

SEQ ID NO: 507 is UCAUGUUUAAUAGCCAAGGAUGACUUGCCUGAUCUUUUUCACCUCCAUGAUUCA AUUUUAAGUUCGUGGAUUUUGGAUUAUUAUGCGUUUAAAAGGUAUAAUAAUUU GAGAUCAUGUUGAAUCUUGCGGGUUAGGUUUCAGGCAGUCUCUUUGGCUAUCUU GACAUG, an RNA fragment encoding a hairpin motif, the fragment arising from A. thaliana;

SEQ ID NO: 508 is UCAUAUUUGGUAGCCAAGGAUGACUUGCCUGUUUCUUUGAGUAAAAUGGGUUAG UGUCAUGUUUGACAAGUGACUAUAAGUUAUAUCAAGCAAUGACCAUUUUACUCA UCAAAAGACAUCAGGCAGUCUCCUUGGCUAUCCUUAUAUG, an RNA fragment encoding a hairpin motif, the fragment arising from A. thaliana;

SEQ ID NO: 509 is UCAUGUUUAGUAGCCAAGGAUGACUUGCCUGAUCUUUUUCGCCUCCACGAUUCA AUUUCAAAUUCAUGCAUUUUGGAUUAUUAUACCUUUUAAAGUAUAAUAGGUCAA AUAUCAUGUUGAAUCUUGCGGGUUAGGUUUCAGGCAGUCUCUUUGGCUAUCUUG ACAUG, an RNA fragment encoding a hairpin motif, the fragment arising from A. thaliana;

SEQ ID NO: 510 is GAGUCCCUCUGAUAUUGGCCUGGUUCACUCAGAUUCUCUUUUACUAACUCAUCU GAUUGAGCCGUGUCAAUAUCUCAGUCCUCU, an RNA fragment encoding a hairpin motif, the fragment arising from A. thaliana;

SEQ ID NO: 511 is GAGUCCCUUUGAUAUUGGCCUGGUUCACUCAGAUCUUACCUGACCACACACGUA GAUAUACAUUAUUCUCUCUAGAUUAUCUGAUUGAGCCGCGCCAAUAUCUCAGUA CUCU, an RNA fragment encoding a hairpin motif, the fragment arising from A. thaliana;

SEQ ID NO: 512 is GGUAACGCGAGAUAUUAGUGCGGUUCAAUCAAAUAGUCGUCCUCUUAACUCAUG GAGAACGGUGUUGUUCGAUUGAGCCGUGCCAAUAUCACGCGGUAAA, an RNA fragment encoding a hairpin motif, the fragment arising from A. thaliana;

SEQ ID NO: 513 is UCAAAUACGAGAUAUUGGUGCGGUUCAAUCAGAAAACCGUACUCUUUUGUUUUA AAGAUCGGUUUAUUUGAUUGAGCCGUGCCAAUAUCACGCGUUUAA, an RNA fragment encoding a hairpin motif, the fragment arising from A. thaliana;

SEQ ID NO: 514 is UUGUUGGCUGCUGUGGCAUCAUCAAGAUUCACAUCUGUUGAUGGACGGUGGUGA UUCACUCUCCACAAAGUUCUCUAUGAAAAUGAGAAUCUUGAUGAUGCUGCAUCG GCAAUCAA, an RNA fragment encoding a hairpin motif, the fragment arising from A. thaliana;

SEQ ID NO: 515 is UUGUUUGUAGGCGCAGCACCAUUAAGAUUCACAUGGAAAUUGAUAAAUACCCUA AAUUAGGGUUUUGAUAUGUAUAUGAGAAUCUUGAUGAUGCUGCAUCAACAAUCG A, an RNA fragment encoding a hairpin motif, the fragment arising from A. thaliana;

SEQ ID NO: 516 is CUGUUCGCUGUUGGAGCAUCAUCAAGAUUCACAAAUCAUCAAGUAUUCGUGUAA AUAAACCCAUUUAUGAUUAGAUUUUUGAUGUAUGUAUGAGAAUCUUGAUGAUGC UGCAGCUGCAAUCAG, an RNA fragment encoding a hairpin motif, the fragment arising from A. thaliana;

SEQ ID NO: 517 is UUGUUUGCUAUUGCAACAUCUUCAAGAUUCAGAAAUCAGAUUCUCUUAUGGGUU UUCUUUUGAGCCUUUAUUUUUUGGUUUGAGAAUCUUGAUGAUGCUGCAGCGGCA AUUAA, an RNA fragment encoding a hairpin motif, the fragment arising from A. thaliana;

SEQ ID NO: 518 is GUAGUCGCAGAUGCAGCACCAUUAAGAUUCACAAGAGAUGUGGUUCCCUUUGCU UUCGCCUCUCGAUCCGCAGAAAAGGGUUCCUUAUCGAGUGGGAAUCUUGAUGAU GCUGCAUCAGCAAAUAC, an RNA fragment encoding a hairpin motif, the fragment arising from A. thaliana;

SEQ ID NO: 519 is AUUAAGUACUUUCGCUUGCAGAGAGAAAUCACAGUGGUCAAAAAAGUUGUAGUU UUCUUAAAGUCUCUUUCCUCUGUGAUUCUCUGUGUAAGCGAAAGAGCUUGCU, an RNA fragment encoding a hairpin motif, the fragment arising from A. thaliana;

SEQ ID NO: 520 is AGAGGAAGGAUCCAAAGGGAUCGCAUUGAUCCUAAUUAAGGUGAAUUCUCCCCA UAUUUUCUUUAUAAUUGGCAAAUAAAUCACAAAAAUUUGCUUGGUUUUGGAUCA UGCUAUCUCUUUGGAUUCAUCCUUC, an RNA fragment encoding a hairpin motif, the fragment arising from A. thaliana;

SEQ ID NO: 521 is AGAGAAAGGAUCCAAAGGGAUCGCAUUGAUCCUAAUUAAGCUGAUUUAUUCCCC AAUAAUUGUUUUUUUUUUCCUUCUCAAUCGAAAGAUGGAAGAAAAACAAAUUCC AAACAUUUUGCUUACUUUUCCGGAUCAUGCGAUCUCUUUGGAUUCAUUCUUU, an RNA fragment encoding a hairpin motif, the fragment arising from A. thaliana;

SEQ ID NO: 522 is CUUACAGUCAUCUUUGGCAUUCUGUCCACCUCCUUCUAUACAUAUAUGCAUGUG UAUAUAUAUAUGCGUUUCGUGUGAAAGAAGGAGGUGGGUAUACUGCCAAUAGAG AUCUGUUAG, an RNA fragment encoding a hairpin motif, the fragment arising from A. thaliana;

SEQ ID NO: 523 is CUUACAGAGAUCUUUGGCAUUCUGUCCACCUCCUCUCUCUAUAUUUAUGUGUAA UAAGUGUACGUAUCUACGGUGUGUUUCGUAAGAGGAGGUGGGCAUACUGCCAAU AGAGAUCUGUUAG, an RNA fragment encoding a hairpin motif, the fragment arising from A. thaliana;

SEQ ID NO: 524 is AUGUCUCCUAGAGUUCCUCUGAGCACUUCAUUGGGGAUACAAUUUUUCUAAAUG AUUAUCCACUGAAGUGUUUGGGGGAACUCCCGGACCCAU, an RNA fragment encoding a hairpin motif, the fragment arising from A. thaliana;

SEQ ID NO: 525 is AUGUCCCCAUGAGUUCCCUUUAACGCUUCAUUGUUAAAUACUCAAAGCCACAUU GGUUUGUAUACAACACUGAAGUGUUUGGGGGGACUCUUGGUGUCAU, an RNA fragment encoding a hairpin motif, the fragment arising from A. thaliana;

SEQ ID NO: 526 is AUGUCCACAUGAGUUCCCUUUAACGCUUCAUUGUUGAAUACUCAAAGCCACAUU GGUUUGUAUAUAACACUGAAGUGUUUGGGGGGACUCUUGGUGUCAU, an RNA fragment encoding a hairpin motif, the fragment arising from A. thaliana;

SEQ ID NO: 527 is AUGUCCUCUAGAGUUCUCCUGAACACUUCAUUGGAAAUUUGUUAUUCAGUAAGC UAACAGUUAAUUCCACUGAAGUGUUUGGGGGAACUCCCGAUGUCAU, an RNA fragment encoding a hairpin motif, the fragment arising from A. thaliana;

SEQ ID NO: 528 is AUGUUUUCUAGAGUUCCUCUGAGCACUUCAUUGGAGAUACAAUUUUUUAUAAAA UAGUUUUCUACUGAAGUGUUUGGGGGAACUCCCGGGCUGAU, an RNA fragment encoding a hairpin motif, the fragment arising from A. thaliana;

SEQ ID NO: 529 is AUGUCCCCUUGAGUUCCCUUAAACGCUUCAUUGUUCAUACUUUGUUAUCAUCUA UCGAUCGAUCAAUCAAUCUGAUGAACACUGAAGUGUUUGGGGGGACUCUAGGUG ACAU, an RNA fragment encoding a hairpin motif, the fragment arising from A. thaliana;

SEQ ID NO:530is CUCUGUAUUCUUCCACAGCUUUCUUGAACUGCAAAACUUCUUCAGAUUUUUUUU UUUUUCUUUUGAUAUCUCUUACGCAUAAAAUAGUGAUUUUCUUCAUAUCUCUGC UCGAUUGAUUUGCGGUUCAAUAAAGCUGUGGGAAGAUACAGAC, an RNA fragment encoding a hairpin motif, the fragment arising from A. thaliana;

SEQ ID NO: 531 is GGUCAUACUUUUCCACAGCUUUCUUGAACUUUCUUUUUCAUUUCCAUUGUUUUU UUCUUAAACAAAAGUAAGAAGAAAAAAAACUUUAAGAUUAAGCAUUUUGGAAGC UCAAGAAAGCUGUGGGAAAACAUGACA, an RNA fragment encoding a hairpin motif, the fragment arising from A. thaliana;

SEQ ID NO: 532 is UGAAUGAACAUCAUUGAGUGCAGCGUUGAUGUAAUUUCGUUUUGUUUUUCAUUG UUGAAUGGAUUAAAAGAAUUUAUACCAGCGUUGCGCUCAAUUAUGUUUUUCUA, an RNA fragment encoding a hairpin motif, the fragment arising from A. thaliana;

SEQ ID NO: 533 is UGAAUGAACAUCAUUGAGUGCAUCGUUGAUGUAAUUUUACUUAUUUUAUUCCAU UGUUGAAUUAAUUAAAGAAGUAUAUAUCAGCGUUGCAUUCAAUUAUGUUUUUCU A, an RNA fragment encoding a hairpin motif, the fragment arising from A. thaliana;

SEQ ID NO: 534 is UGAAAUUUCAAAGGAGUGGCAUGUGAACACAUAUCCUAUGGUUUCUUCAAAUUU CCAUUGAAACCAUUGAGUUUUGUGUUCUCAGGUCACCCCUUUGAAUCUCCC, an RNA fragment encoding a hairpin motif, the fragment arising from A. thaliana;

SEQ ID NO:535is UGGAUCUCGACAGGGUUGAUAUGAGAACACACGAGUAAUCAACGGCUGUAAUGA CGCUACGUCAUUGUUACAGCUCUCGUUUUCAUGUGUUCUCAGGUCACCCCUGCUG AGCUCUU, an RNA fragment encoding a hairpin motif, the fragment arising from A. thaliana;

SEQ ID NO: 536 is UGGAUCUCGACAGGGUUGAUAUGAGAACACACGAGCAAUCAACGGCUAUAACGA CGCUACGUCAUUGUUACAGCUCUCGUUCAUGUGUUCUCAGGUCACCCCUGCUGA GCUCUU, an RNA fragment encoding a hairpin motif, the fragment arising from A. thaliana;

SEQ ID NO: 537 is AAAUGCAUUACAGGGUAAGAUCUCUAUUGGCAGGAAACCAUUACUUAGAUCUUU GCAUCUCUUUAUGCAUUGCUUUUAAUUAGUGAGUUAUCUGCCAAAGGAGAUUUG CCCUGUAAUUCUUCU, an RNA fragment encoding a hairpin motif, the fragment arising from A. thaliana;

SEQ ID NO: 538 is UCACUAGUUUUAGGGCGCCUCUCCAUUGGCAGGUCCUUUACUUCCAAAUAUACA CAUACAUAUAUGAAUAUCGAAAAUUUCCGAUGAUCGAUUUAUAAAUGACCUGCC AAAGGAGAGUUGCCCUGAAACUGGUUC, an RNA fragment encoding a hairpin motif, the fragment arising from A. thaliana;

SEQ ID NO: 539 is GGAGCAGUAAUAGGGCAUCUUUCUAUUGGCAGGCGACUUGGCUAUUUGUAUCUU UUGUGUUCUUGACUAUUGGCUAUGUCACUUGCCAAAGGAGAGUUGCCCUGUCAC UGCUUC, an RNA fragment encoding a hairpin motif, the fragment arising from A. thaliana;

SEQ ID NO: 540 is GGUUGGAUUACUGGGCGAAUACUCCUAUGGCAGAUCGCAUUGGCUAGAUAUGCA AGUAAAAUGCUUCUCUGCCAAAGGAGAUUUGCCCCGCAAUUCAUCC, an RNA fragment encoding a hairpin motif, the fragment arising from A. thaliana;

SEQ ID NO: 541 is GAAAGCAUUACAGGGCGAAUCCUCUAUUGGCAGUGGAAGUUGAUGACCCUUAUA UGUUAUUUUCUCAUCAUUUUCCUCUGCCAAAGGAGAUUUGCCUCGCAAUGCUUC A, an RNA fragment encoding a hairpin motif, the fragment arising from A. thaliana;

SEQ ID NO: 542 is AUAUGCAUUACAGGGCAAGAUCACCAUUGGCAGAGAUCUAUUACUUCAUUCUUG CAUCAUAUGCAUAAAUGUUUGUGGUGAGCUCUCUGCCAAAGGAGAUUUGCCCGG UAAUUCUCUU, an RNA fragment encoding a hairpin motif, the fragment arising from A. thaliana;

SEQ ID NO: 543 is UGACAGAAGAGAGUGAGCACA, an miRNA sequence arising from O. sativa;

SEQ ID NO: 544 is UGACAGAAGAGAGUGAGCACA, an miRNA sequence arising from O. sativa;

SEQ ID NO: 545 is UGACAGAAGAGAGUGAGCACA, an miRNA sequence arising from O. sativa;

SEQ ID NO: 546 is UGACAGAAGAGAGUGAGCACA, an miRNA sequence arising from O. sativa;

SEQ ID NO: 547 is UGACAGAAGAGAGUGAGCACA, an miRNA sequence arising from O. sativa;

SEQ ID NO: 548 is UGACAGAAGAGAGUGAGCACA, an miRNA sequence arising from O. sativa;

SEQ ID NO: 549 is UGACAGAAGAGAGUGAGCACA, an miRNA sequence arising from O. sativa;

SEQ ID NO: 550 is UGACAGAAGAGAGUGAGCACA, an miRNA sequence arising from O. sativa;

SEQ ID NO: 551 is UGACAGAAGAGAGUGAGCACA, an miRNA sequence arising from O. sativa;

SEQ ID NO: 552 is UGACAGAAGAGAGUGAGCACA, an miRNA sequence arising from O. sativa;

SEQ ID NO: 553 is UGACAGAAGAGAGAGAGCACA, an miRNA sequence arising from O. sativa;

SEQ ID NO: 554 is CGACAGAAGAGAGUGAGCAUA, an miRNA sequence arising from O. sativa;

SEQ ID NO: 555 is UUUGGAUUGAAGGGAGCUCUG, an miRNA sequence arising from O. sativa;

SEQ ID NO: 556 is UUUUGGAUUGAAGGGAGCUCUG, an miRNA sequence arising from O. sativa;

SEQ ID NO: 557 is AUUGGAUUGAAGGGAGCUCCA, an miRNA sequence arising from O. sativa;

SEQ ID NO: 558 is AUUGGAUUGAAGGGAGCUCCG, an miRNA sequence arising from O. sativa;

SEQ ID NO: 559 is AUUGGAUUGAAGGGAGCUCCU, an miRNA sequence arising from O. sativa;

SEQ ID NO: 560 is CUUGGAUUGAAGGGAGCUCUA, an miRNA sequence arising from O. sativa;

SEQ ID NO: 561 is UUGGACUGAAGGGUGCUCCC, an miRNA sequence arising from O. sativa;

SEQ ID NO: 562 is UUGGACUGAAGGGUGCUCCC, an miRNA sequence arising from O. sativa;

SEQ ID NO: 563 is UGCCUGGCUCCCUGUAUGCCA, an miRNA sequence arising from O. sativa;

SEQ ID NO: 564 is UGCCUGGCUCCCUGUAUGCCA, an miRNA sequence arising from O. sativa;

SEQ ID NO: 565 is UGCCUGGCUCCCUGUAUGCCA, an miRNA sequence arising from O. sativa;

SEQ ID NO: 566 is UGCCUGGCUCCCUGUAUGCCA, an miRNA sequence arising from O. sativa;

SEQ ID NO: 567 is UGCCUGGCUCCCUGUAUGCCG, an miRNA sequence arising from O. sativa;

SEQ ID NO: 568 is UGCCUGGCUCCCUGAAUGCCA, an miRNA sequence arising from O. sativa;

SEQ ID NO: 569 is UCGAUAAACCUCUGCAUCCAG, an miRNA sequence arising from O. sativa;

SEQ ID NO: 570 is UCGAUAAGCCUCUGCAUCCAG, an miRNA sequence arising from O. sativa;

SEQ ID NO: 571 is UGGAGAAGCAGGGCACGUGCA, an miRNA sequence arising from O. sativa;

SEQ ID NO: 572 is UGGAGAAGCAGGGCACGUGCA, an miRNA sequence arising from O. sativa;

SEQ ID NO: 573 is UGGAGAAGCAGGGUACGUGCA, an miRNA sequence arising from O. sativa;

SEQ ID NO: 574 is UGGAGAAGCAGGGCACGUGCU, an miRNA sequence arising from O. sativa;

SEQ ID NO: 575 is UGGAGAAGCAGGGCACGUGAG, an miRNA sequence arising from O. sativa;

SEQ ID NO: 576 is UCGGACCAGGCUUCAUUCCCC, an miRNA sequence arising from O. sativa;

SEQ ID NO: 577 is UCGGACCAGGCUUCAUUCCCC, an miRNA sequence arising from O. sativa;

SEQ ID NO: 578 is UCGGACCAGGCUUCAUUCCCC, an miRNA sequence arising from O. sativa;

SEQ ID NO: 579 is UCGGACCAGGCUUCAUUCCCC, an miRNA sequence arising from O. sativa;

SEQ ID NO: 580 is UCGGACCAGGCUUCAUUCCCC, an miRNA sequence arising from O. sativa;

SEQ ID NO: 581 is UCGGACCAGGCUUCAUUCCCC, an miRNA sequence arising from O. sativa;

SEQ ID NO: 582 is UCGGACCAGGCUUCAUUCCUC, an miRNA sequence arising from O. sativa;

SEQ ID NO: 583 is UCGGACCAGGCUUCAUUCCUC, an miRNA sequence arising from O. sativa;

SEQ ID NO: 584 is UCGGAUCAGGCUUCAUUCCUC, an miRNA sequence arising from O. sativa;

SEQ ID NO: 585 is UCGGAUCAGGCUUCAUUCCUC, an miRNA sequence arising from O. sativa;

SEQ ID NO: 586 is UCGGACCAGGCUUCAAUCCCU, an miRNA sequence arising from O. sativa;

SEQ ID NO: 587 is UCGGACCAGGCUUCAAUCCCU, an miRNA sequence arising from O. sativa;

SEQ ID NO: 588 is UGAAGCUGCCAGCAUGAUCUA, an miRNA sequence arising from O. sativa;

SEQ ID NO: 589 is UGAAGCUGCCAGCAUGAUCUA, an miRNA sequence arising from O. sativa;

SEQ ID NO: 590 is UGAAGCUGCCAGCAUGAUCUA, an miRNA sequence arising from O. sativa;

SEQ ID NO: 591 is UGAAGCUGCCAGCAUGAUCUG, an miRNA sequence arising from O. sativa;

SEQ ID NO: 592 is UGAAGCUGCCAGCAUGAUCUG, an miRNA sequence arising from O. sativa;

SEQ ID NO: 593 is UGAAGCUGCCAGCAUGAUCUG, an miRNA sequence arising from O. sativa;

SEQ ID NO: 594 is UGAAGCUGCCAGCAUGAUCUG, an miRNA sequence arising from O. sativa;

SEQ ID NO: 595 is UGAAGCUGCCAGCAUGAUCUG, an miRNA sequence arising from O. sativa;

SEQ ID NO: 596 is UGAAGCUGCCAGCAUGAUCUG, an miRNA sequence arising from O. sativa;

SEQ ID NO: 597 is UCGCUUGGUGCAGAUCGGGAC, an miRNA sequence arising from O. sativa;

SEQ ID NO: 598 is AGGCUUGGUGCAGCUCGGGAA, an miRNA sequence arising from O. sativa;

SEQ ID NO: 599 is CAGCCAAGGAUGACUUGCCGA, an miRNA sequence arising from O. sativa;

SEQ ID NO: 600 is CAGCCAAGGAUGACUUGCCGG, an miRNA sequence arising from O. sativa;

SEQ ID NO: 601 is CAGCCAAGGAUGACUUGCCGG, an miRNA sequence arising from O. sativa;

SEQ ID NO: 602 is UAGCCAAGGAUGAAUUGCCGG, an miRNA sequence arising from O. sativa;

SEQ ID NO: 603 is UAGCCAAGGAUGACUUGCCGG, an miRNA sequence arising from O. sativa;

SEQ ID NO: 604 is UAGCCAAGGAUGACUUGCCUA, an miRNA sequence arising from O. sativa;

SEQ ID NO: 605 is UAGCCAAGGAUGACUUGCCUA, an miRNA sequence arising from O. sativa;

SEQ ID NO: 606 is UAGCCAAGGAUGACUUGCCUG, an miRNA sequence arising from O. sativa;

SEQ ID NO: 607 is UAGCCAAGGAUGACUUGCCUG, an miRNA sequence arising from O. sativa;

SEQ ID NO: 608 is UAGCCAAGGAUGACUUGCCUG, an miRNA sequence arising from O. sativa;

SEQ ID NO: 609 is UAGCCAAGGAUGACUUGCCUG, an miRNA sequence arising from O. sativa;

SEQ ID NO: 610 is UAGCCAAGGAUGACUUGCCUG, an miRNA sequence arising from O. sativa;

SEQ ID NO: 611 is UAGCCAAGGAUGACUUGCCUG, an miRNA sequence arising from O. sativa;

SEQ ID NO: 612 is UAGCCAAGAAUGACUUGCCUA, an miRNA sequence arising from O. sativa;

SEQ ID NO: 613 is UAGCCAAGAAUGACUUGCCUA, an miRNA sequence arising from O. sativa;

SEQ ID NO: 614 is UAGCCAAGGACAAACUUGCCGG, an miRNA sequence arising from O. sativa;

SEQ ID NO: 615 is UAGCCAAGGAGACUGCCCAUG, an miRNA sequence arising from O. sativa;

SEQ ID NO: 616 is UGAUUGAGCCGCGCCAAUAUC, an miRNA sequence arising from O. sativa;

SEQ ID NO: 617 is UGAUUGAGCCGUGCCAAUAUC, an miRNA sequence arising from O. sativa;

SEQ ID NO: 618 is UGAUUGAGCCGUGCCAAUAUC, an miRNA sequence arising from O. sativa;

SEQ ID NO: 619 is UGAUUGAGCCGUGCCAAUAUC, an miRNA sequence arising from O. sativa;

SEQ ID NO: 620 is UGAUUGAGCCGUGCCAAUAUC, an miRNA sequence arising from O. sativa;

SEQ ID NO: 621 is UGAUUGAGCCGUGCCAAUAUC, an miRNA sequence arising from O. sativa;

SEQ ID NO: 622 is GAGGUGAGCCGAGCCAAUAUC, an miRNA sequence arising from O. sativa;

SEQ ID NO: 623 is AGAAUCUUGAUGAUGCUGCAU, an miRNA sequence arising from O. sativa;

SEQ ID NO: 624 is GGAAUCUUGAUGAUGCUGCAU, an miRNA sequence arising from O. sativa;

SEQ ID NO: 625 is UGAAUCUUGAUGAUGCUGCAC, an miRNA sequence arising from O. sativa;

SEQ ID NO: 626 is UCCAAAGGGAUCGCAUUGAUC, an miRNA sequence arising from O. sativa;

SEQ ID NO: 627 is UUGGCAUUCUGUCCACCUCC, an miRNA sequence arising from O. sativa;

SEQ ID NO: 628 is GUGAAGUGCUUGGGGGAACUC, an miRNA sequence arising from O. sativa;

SEQ ID NO: 629 is GUGAAGUGUUUGGGGGAACUC, an miRNA sequence arising from O. sativa;

SEQ ID NO: 630 is GUGAAGUGUUUGGAGGAACUC, an miRNA sequence arising from O. sativa;

SEQ ID NO: 631 is GUGAAGUGUUUGGGGGAACUC, an miRNA sequence arising from O. sativa;

SEQ ID NO: 632 is GUGAAGUGUUUGGGGGAACUC, an miRNA sequence arising from O. sativa;

SEQ ID NO: 633 is GUGAAGUAUUUGGGGGAACUC, an miRNA sequence arising from O. sativa;

SEQ ID NO: 634 is GUGAAGUGUUUGGGGGAACUC, an miRNA sequence arising from O. sativa;

SEQ ID NO: 635 is GUGAAGUGUUUGGGGGAACUC, an miRNA sequence arising from O. sativa;

SEQ ID NO: 636 is GUGAAGUGUUUGGGGGAACUC, an miRNA sequence arising from O. sativa;

SEQ ID NO: 637 is GUGAAGUGUUUGGGGGAACUC, an miRNA sequence arising from O. sativa;

SEQ ID NO: 638 is GUGAAGUGUUUGGGGGAACUC, an miRNA sequence arising from O. sativa;

SEQ ID NO: 639 is GUGAAGUGUUUGGGGGAACUC, an miRNA sequence arising from O. sativa;

SEQ ID NO: 640 is GUGAAGUGUUUGGGGGAACUC, an miRNA sequence arising from O. sativa;

SEQ ID NO: 641 is GUGAAGUGUUUGGGGGAACUC, an miRNA sequence arising from O. sativa;

SEQ ID NO: 642 is GUGAAGUGUUUGGGGAAACUC, an miRNA sequence arising from O. sativa;

SEQ ID NO: 643 is GUGAAGCGUUUGGGGGAAAUC, an miRNA sequence arising from O. sativa;

SEQ ID NO: 644 is GUGAAGUGUUUGGGGAAACUC, an miRNA sequence arising from O. sativa;

SEQ ID NO: 645 is GUGAAGUGUUUGGGGAAACUC, an miRNA sequence arising from O. sativa;

SEQ ID NO: 646 is GUGAAGCGUUUGGGGGAAAUC, an miRNA sequence arising from O. sativa;

SEQ ID NO: 647 is UUCCACAGCUUUCUUGAACUG, an miRNA sequence arising from O. sativa;

SEQ ID NO: 648 is UUCCACAGCUUUCUUGAACUG, an miRNA sequence arising from O. sativa;

SEQ ID NO: 649 is UUCCACAGCUUUCUUGAACUU, an miRNA sequence arising from O. sativa;

SEQ ID NO: 650 is UCAUUGAGUGCAGCGUUGAUG, an miRNA sequence arising from O. sativa;

SEQ ID NO: 651 is UUAUUGAGUGCAGCGUUGAUG, an miRNA sequence arising from O. sativa;

SEQ ID NO: 652 is UGUGUUCUCAGGUCACCCCUU, an miRNA sequence arising from O. sativa;

SEQ ID NO: 653 is UGUGUUCUCAGGUCGCCCCUG, an miRNA sequence arising from O. sativa;

SEQ ID NO: 654 is UGCCAAAGGAGAAUUGCCCUG, an miRNA sequence arising from O. sativa;

SEQ ID NO: 655 is UGCCAAAGGAGAAUUGCCCUG, an miRNA sequence arising from O. sativa;

SEQ ID NO: 656 is UGCCAAAGGAGAAUUGCCCUG, an miRNA sequence arising from O. sativa;

SEQ ID NO: 657 is UGCCAAAGGAGAGUUGCCCUG, an miRNA sequence arising from O. sativa;

SEQ ID NO: 658 is UGCCAAAGGAGAUUUGCCCAG, an miRNA sequence arising from O. sativa;

SEQ ID NO: 659 is UGCCAAAGGAGAUUUGCCCAG, an miRNA sequence arising from O. sativa;

SEQ ID NO: 660 is UGCCAAAGGAGAUUUGCCCGG, an miRNA sequence arising from O. sativa;

SEQ ID NO: 661 is UGCCAAAGGAGACUUGCCCAG, an miRNA sequence arising from O. sativa;

SEQ ID NO: 662 is UGCCAAAGGAGAGCUGCCCUG, an miRNA sequence arising from O. sativa;

SEQ ID NO: 663 is UGCCAAAGGAGAGUUGCCCUA, an miRNA sequence arising from O. sativa;

SEQ ID NO: 664 is UGCCAAAGGAAAUUUGCCCCG, an miRNA sequence arising from O. sativa;

SEQ ID NO: 665 is GGUGACAGAAGAGAGUGAGCACACGUGGUUG, a sequence expected to encode miRNA an arising from O. sativa;

SEQ ID NO: 666 is GUCUGACAGAAGAGAGUGAGCACACACGGUGC, a sequence expected to encode miRNA an arising from O. sativa;

SEQ ID NO: 667 is GGCUGACAGAAGAGAGUGAGCACACAUGGUGA, a sequence expected to encode miRNA an arising from O. sativa;

SEQ ID NO: 668 is UUGACAGAAGAGAGUGAGCACACAGCGUG, a sequence expected to encode miRNA an arising from O. sativa;

SEQ ID NO: 669 is GGUGACAGAAGAGAGUGAGCACACGG, a sequence expected to encode miRNA an arising from O. sativa;

SEQ ID NO: 670 is AGUUGACAGAAGAGAGUGAGCACAC, a sequence expected to encode miRNA an arising from O. sativa;

SEQ ID NO: 671 is GGCUGACAGAAGAGAGUGAGCACACAGCGGG, a sequence expected to encode miRNA an arising from O. sativa;

SEQ ID NO: 672 is UUGUUGACAGAAGAGAGUGAGCACACGG, a sequence expected to encode miRNA an arising from O. sativa;

SEQ ID NO: 673 is GGUGACAGAAGAGAGUGAGCACACGG, a sequence expected to encode miRNA an arising from O. sativa;

SEQ ID NO: 674 is UUGUUGACAGAAGAGAGUGAGCACACGG, a sequence expected to encode miRNA an arising from O. sativa;

SEQ ID NO: 675 is UGACAGAAGAGAGAGAGCAC, a sequence expected to encode miRNA an arising from O. sativa;

SEQ ID NO: 676 is GCCGACAGAAGAGAGUGAGCAUAUAU, a sequence expected to encode miRNA an arising from O. sativa;

SEQ ID NO: 677 is UCUUUGGAUUGAAGGGAGCUCUG, a sequence expected to encode miRNA an arising from O. sativa;

SEQ ID NO: 678 is ACUCUUUGGAUUGAAGGGAGCUCUG, a sequence expected to encode miRNA an arising from O. sativa;

SEQ ID NO: 679 is UGAUUGGAUUGAAGGGAGCUCCAC, a sequence expected to encode miRNA an arising from O. sativa;

SEQ ID NO: 680 is UGAUUGGAUUGAAGGGAGCUCC, a sequence expected to encode miRNA an arising from O. sativa;

SEQ ID NO: 681 is UUGAUUGGAUUGAAGGGAGCUCCU, a sequence expected to encode miRNA an arising from O. sativa;

SEQ ID NO: 682 is UUAUGCUUGGAUUGAAGGGAGCUCUA, a sequence expected to encode miRNA an arising from O. sativa;

SEQ ID NO: 683 is UGGUUGGACUGAAGGGUGCUC, a sequence expected to encode miRNA an arising from O. sativa;

SEQ ID NO: 684 is GUGUGCCUGGCUCCCUGUAUGCCACACA, a sequence expected to encode miRNA an arising from O. sativa;

SEQ ID NO: 685 is AGCGUGCCUGGCUCCCUGUAUGCCACUC, a sequence expected to encode miRNA an arising from O. sativa;

SEQ ID NO: 686 is AUGUGCCUGGCUCCCUGUAUGCCACUC, a sequence expected to encode miRNA an arising from O. sativa;

SEQ ID NO: 687 is GAUAUGCCUGGCUCCCUGUAUGCCACUCG, a sequence expected to encode miRNA an arising from O. sativa;

SEQ ID NO: 688 is GGAUAUGCCUGGCUCCCUGUAUGCCGC, a sequence expected to encode miRNA an arising from O. saliva;

SEQ ID NO: 689 is CUGCCUGGCUCCCUGAAUGCCAUC, a sequence expected to encode miRNA an arising from O. saliva;

SEQ ID NO: 690 is GGAAUCGAUCGAUAAACCUCUGCAUCCAGU, a sequence expected to encode miRNA an arising from O. saliva;

SEQ ID NO: 691 is GAAUCGAUCGAUAAGCCUCUGCAUCCAGA, a sequence expected to encode miRNA an arising from O. saliva;

SEQ ID NO: 692 is ACGGUGGAGAAGCAGGGCACGUGCA, a sequence expected to encode miRNA an arising from O. saliva;

SEQ ID NO: 693 is CCGCGUUGGAGAAGCAGGGCACGUGCAUGC, a sequence expected to encode miRNA an arising from O. saliva;

SEQ ID NO: 694 is UUGUUGGGAGAAGCAGGGUACGUGCAA, a sequence expected to encode miRNA an arising from O. saliva;

SEQ ID NO: 695 is CCGUGCUGGAGAAGCAGGGCACGUGCUC, a sequence expected to encode miRNA an arising from O. saliva;

SEQ ID NO: 696 is AGGGUGGAGAAGCAGGGCACGUGAGC, a sequence expected to encode miRNA an arising from O. saliva;

SEQ ID NO: 697 is UCUCGGACCAGGCUUCAUUCCCCUCAGA, a sequence expected to encode miRNA an arising from O. saliva;

SEQ ID NO: 698 is UCUCGGACCAGGCUUCAUUCCCCCC, a sequence expected to encode miRNA an arising from O. saliva;

SEQ ID NO: 699 is UUCCGGACCAGGCUUCAUUCCCCCC, a sequence expected to encode miRNA an arising from O. saliva;

SEQ ID NO: 700 is UCUCGGACCAGGCUUCAUUCCCCUCAAGU, a sequence expected to encode miRNA an arising from O. saliva;

SEQ ID NO: 701 is UCUCGGACCAGGCUUCAUUCCCCUCAGA, a sequence expected to encode miRNA an arising from O. saliva;

SEQ ID NO: 702 is UCUCGGACCAGGCUUCAUUCCCCUCAACA, a sequence expected to encode miRNA an arising from O. saliva;

SEQ ID NO: 703 is UCUCGGACCAGGCUUCAUUCCUCACA, a sequence expected to encode miRNA an arising from O. saliva;

SEQ ID NO: 704 is UCGGACCAGGCUUCAUUCCUCGCAA, a sequence expected to encode miRNA an arising from O. saliva;

SEQ ID NO: 705 is GGAGCCUCGGACCAGGCUUCAAUCCCUU, a sequence expected to encode miRNA an arising from O. sativa;

SEQ ID NO: 706 is CUCGGACCAGGCUUCAAUCCCUU, a sequence expected to encode miRNA an arising from O. sativa;

SEQ ID NO: 707 is GAGUGAAGCUGCCAGCAUGAUCUAGCUCUG, a sequence expected to encode miRNA an arising from O. sativa;

SEQ ID NO: 708 is CGUGAAGCUGCCAGCAUGAUCUAACUU, a sequence expected to encode miRNA an arising from O. sativa;

SEQ ID NO: 709 is GAGUGAAGCUGCCAGCAUGAUCUAGCUC, a sequence expected to encode miRNA an arising from O. sativa;

SEQ ID NO: 710 is AGCUGAAGCUGCCAGCAUGAUCUGAUGA, a sequence expected to encode miRNA an arising from O. sativa;

SEQ ID NO: 711 is AUGAAGCUGCCAGCAUGAUCUGGU, a sequence expected to encode miRNA an arising from O. sativa;

SEQ ID NO: 712 is UGGAUGAAGCUGCCAGCAUGAUCUGAUCA, a sequence expected to encode miRNA an arising from O. sativa;

SEQ ID NO: 713 is GGUGAAGCUGCCAGCAUGAUCU, a sequence expected to encode miRNA an arising from O. sativa;

SEQ ID NO: 714 is UUGGUGAAGCUGCCAGCAUGAUCUGAUGA, a sequence expected to encode miRNA an arising from O. sativa;

SEQ ID NO: 715 is GGCUGAAGCUGCCAGCAUGAUCUGGU, a sequence expected to encode miRNA an arising from O. sativa;

SEQ ID NO: 716 is CUCGGGCUCGCUUGGUGCAGAUCGGGACCC, a sequence expected to encode miRNA an arising from O. sativa;

SEQ ID NO: 717 is AUGGUGCAGCCAAGGAUGACUUGCCGAUC, a sequence expected to encode miRNA an arising from O. sativa;

SEQ ID NO: 718 is AAUGCAGCCAAGGAUGACUUGCCGGU, a sequence expected to encode miRNA an arising from O. sativa;

SEQ ID NO: 719 is GGAUGCAGCCAAGGAUGACUUGCCGGCUC, a sequence expected to encode miRNA an arising from O. sativa;

SEQ ID NO: 720 is GUGUAGCCAAGGAUGAAUUGCCGGC, a sequence expected to encode miRNA an arising from O. sativa;

SEQ ID NO: 721 is UUCGGUAGCCAAGGAUGACUUGCCU, a sequence expected to encode miRNA an arising from O. sativa;

SEQ ID NO: 722 is CUCUGGUAGCCAAGGAUGACUU, a sequence expected to encode miRNA an arising from O. saliva;

SEQ ID NO: 723 is CUCUGGUAGCCAAGGAUGACUU, a sequence expected to encode miRNA an arising from O. saliva;

SEQ ID NO: 724 is CUCUGGUAGCCAAGGAUGACUU, a sequence expected to encode miRNA an arising from O. saliva;

SEQ ID NO: 725 is AUCUGGUAGCCAAGGAUGACUU, a sequence expected to encode miRNA an arising from O. saliva;

SEQ ID NO: 726 is UAGAUAGCCAAGGAUGACUU, a sequence expected to encode miRNA an arising from O. saliva;

SEQ ID NO: 727 is CCUGGUAGCCAAGGAUGACUU, a sequence expected to encode miRNA an arising from O. saliva;

SEQ ID NO: 728 is UUUGGUAGCCAAGAAUGACUU, a sequence expected to encode miRNA an arising from O. saliva;

SEQ ID NO: 729 is UUUGGUAGCCAAGAAUGACUU, a sequence expected to encode miRNA an arising from O. saliva;

SEQ ID NO: 730 is AGCAAGGUGUAGCCAAGGACA, a sequence expected to encode miRNA an arising from O. saliva;

SEQ ID NO: 731 is UCAGGCUAGCCAAGGAGACUG, a sequence expected to encode miRNA an arising from O. saliva;

SEQ ID NO: 732 is GUAUCUGAUUGAGCCGCGCCAAUAUCUC, a sequence expected to encode miRNA an arising from O. saliva;

SEQ ID NO: 733 is UCUUUUGAUUGAGCCGUGCCAAUAUCACGUC, a sequence expected to encode miRNA an arising from O. saliva;

SEQ ID NO: 734 is CUCUUUGAUUGAGCCGUGCCAAUAUCACGUC, a sequence expected to encode miRNA an arising from O. sativa;

SEQ ID NO: 735 is UUCUGAUUGAGCCGUGCCAAUAUCUCAGC, a sequence expected to encode miRNA an arising from O. saliva;

SEQ ID NO: 736 is UUUCUGAUUGAGCCGUGCCAAUAUCUUAG, a sequence expected to encode miRNA an arising from O. saliva;

SEQ ID NO: 737 is GUCUGAUUGAGCCGUGCCAAUAUCAC, a sequence expected to encode miRNA an arising from O. saliva;

SEQ ID NO: 738 is GUGAGCCGAGCCAAUAUCAC, a sequence expected to encode miRNA an arising from O. saliva;

SEQ ID NO: 739 is GGCUGAGAAUCUUGAUGAUGCUGCAUCCGCA, a sequence expected to encode miRNA an arising from O. sativa;

SEQ ID NO: 740 is GGGAAUCUUGAUGAUGCUGCAUCGGAA, a sequence expected to encode miRNA an arising from O. sativa;

SEQ ID NO: 741 is UGCGUGAAUCUUGAUGAUGCUGCACCAGCAA, a sequence expected to encode miRNA an arising from O. sativa;

SEQ ID NO: 742 is GGGGAAGCAUCCAAAGGGAUCGCAUUGAUCCUUC, a sequence expected to encode miRNA an arising from O. sativa;

SEQ ID NO: 743 is GAGAGUUCUUUGGCAUUCUGUCCACCUCCUUG, a sequence expected to encode miRNA an arising from O. sativa;

SEQ ID NO: 744 is AGUGAAGUGCUUGGGGGAACUCCAG, a sequence expected to encode miRNA an arising from O. sativa;

SEQ ID NO: 745 is CGUGAAGUGUUUGGGGGAACUCUUA, a sequence expected to encode miRNA an arising from O. sativa;

SEQ ID NO: 746 is GUGAAGUGUUUGGAGGAACUCUCGG, a sequence expected to encode miRNA an arising from O. sativa;

SEQ ID NO: 747 is UGUGAAGUGUUUGGGGGAACUCUCGGU, a sequence expected to encode miRNA an arising from O. sativa;

SEQ ID NO: 748 is UGUGAAGUGUUUGGGGGAACUCUCGA, a sequence expected to encode miRNA an arising from O. sativa;

SEQ ID NO: 749 is GUGAAGUAUUUGGGGGAACUCUCGA, a sequence expected to encode miRNA an arising from O. sativa;

SEQ ID NO: 750 is UGUGAAGUGUUUGGGGGAACUCUCG, a sequence expected to encode miRNA an arising from O. sativa;

SEQ ID NO: 751 is GUAGUGAAGUGUUUGGGGGAACUCUAGGUGGCA, a sequence expected to encode miRNA an arising from O. sativa;

SEQ ID NO: 752 is UGUGAAGUGUUUGGGGGAACUCUUGGU, a sequence expected to encode miRNA an arising from O. sativa;

SEQ ID NO: 753 is CUGUGAAGUGUUUGGGGGAACUCUAGGUGGCA, a sequence expected to encode miRNA an arising from O. sativa;

SEQ ID NO: 754 is UGUGAAGUGUUUGJGGGGAACUCUUGA, a sequence expected to encode miRNA an arising from O. sativa;

SEQ ID NO: 755 is UUGUGAAGUGUUUGGGGGAACUCUUG, a sequence expected to encode miRNA an arising from O. sativa;

SEQ ID NO: 756 is GUGUUUGGGGGAACUCUCGA, a sequence expected to encode miRNA an arising from O. sativa;

SEQ ID NO: 757 is AGUGAAGUGUUUGGGGAAACUCCGG, a sequence expected to encode miRNA an arising from O. sativa;

SEQ ID NO: 758 is AGUGAAGUGUUUGGGGAAACUCCGG, a sequence expected to encode miRNA an arising from O. sativa;

SEQ ID NO: 759 is AGUGAAGUGUUUGGGGAAACUCCGG, a sequence expected to encode miRNA an arising from O. sativa;

SEQ ID NO: 760 is AUCUUCCACAGCUUUCUUGAACUGC, a sequence expected to encode miRNA an arising from O. sativa;

SEQ ID NO: 761 is GUCUUCCACAGCUUUCUUGAACUGC, a sequence expected to encode miRNA an arising from O. sativa;

SEQ ID NO: 762 is CAUGCCUUUCCACAGCUUUCUUGAACUUCU, a sequence expected to encode miRNA an arising from O. sativa;

SEQ ID NO: 763 is GCAUCAUUGAGUGCAGCGUUGAUGAA, a sequence expected to encode miRNA an arising from O. sativa;

SEQ ID NO: 764 is ACUGUGUUCUCAGGUCACCCCUUUGGG, a sequence expected to encode miRNA an arising from O. sativa;

SEQ ID NO: 765 is CGUGUGUUCUCAGGUCGCCCCUGCCG, a sequence expected to encode miRNA an arising from O. sativa;

SEQ ID NO: 766 is GUGCCAAAGGAGAAUUGCCCUGC, a sequence expected to encode miRNA an arising from O. sativa;

SEQ ID NO: 767 is CGUGCCAAAGGAGAAUUGCCCUGCC, a sequence expected to encode miRNA an arising from O. sativa;

SEQ ID NO: 768 is CGUGCCAAAGGAGAAUUGCCCUGC, a sequence expected to encode miRNA an arising from O. sativa;

SEQ ID NO: 769 is ACCACUGCCAAAGGAGAUUUGCCCAG, a sequence expected to encode miRNA an arising from O. sativa;

SEQ ID NO: 770 is UGUUCUCUCUGCCAAAGGAGAUUUGCCCAG, a sequence expected to encode miRNA an arising from O. sativa;

SEQ ID NO: 771 is UCUGCCAAAGGAGAUUUGCCCGGCGAU, a sequence expected to encode miRNA an arising from O. sativa;

SEQ ID NO: 772 is CCACUGCCAAAGGAGACUUGCCCAGCAA, a sequence expected to encode miRNA an arising from O. sativa;

SEQ ID NO: 773 is CCCUGCCAAAGGAGAGCUGCCCUGCCA, a sequence expected to encode miRNA an arising from O. sativa;

SEQ ID NO: 774 is GGAGAGUUGCCCUAAAACUGGA, a sequence expected to encode miRNA an arising from O. sativa;

SEQ ID NO: 775 is ACUGCCAAAGGAAAUUUGCCCCGGAAUUCA, a sequence expected to encode miRNA an arising from O. sativa;

SEQ ID NO: 776 is ACUAGGAGGGUGACAGAAGAGAGUGAGCACACGUGGUUGUUUCCUUGCAUAAAU GAUGCCUAUGCUUGGAGCUACGCGUGCUCACUUCUCUCUCUGUCACCUCCACCCC, an RNA fragment encoding a hairpin motif, the fragment arising from O. sativa;

SEQ ID NO: 777 is UUUGGAGGUCUGACAGAAGAGAGUGAGCACACACGGUGCUUUCUUAGCAUGCAA GAGCCAUGCUGGGAGCUGUGCGUGCUCACUCUCUAUCUGUCAGCCGUUCACC, an RNA fragment encoding a hairpin motif, the fragment arising from O. sativa;

SEQ ID NO: 778 is GAGGUGAGGCUGACAGAAGAGAGUGAGCACACAUGGUGACUUUCUUGCAUGCUG AAUGGACUCAUGCUUGAAGCUAUGUGUGCUCACUUCUCUCUCUGUCAGCCAUUU GAU, an RNA fragment encoding a hairpin motif, the fragment arising from O. sativa;

SEQ ID NO: 779 is CUCAUGAGAUUGACAGAAGAGAGUGAGCACACAGCGUGAUGGCCGGCAUAAAAU CUAUCCCGUCCUCGCCGCGUGCUCACUCCUCUUUCUGUCACCCUCUUUCU, an RNA fragment encoding a hairpin motif, the fragment arising from O. sativa;

SEQ ID NO: 780 is UGGCGCGAGGUGACAGAAGAGAGUGAGCACACGGCCGGGCGUGACGGCACCGGC GGGCGUGCCGUCGCGGCCGCGUGCUCACUGCUCUUUCUGUCAUCCGGUGCCG, an RNA fragment encoding a hairpin motif, the fragment arising from O. sativa;

SEQ ID NO: 781 is UGGUGGCAGUUGACAGAAGAGAGUGAGCACACAGCGGCCAGACUGCAUCGAUCU AUCAAUCUCCCUUCGACAGGAUAGCUAGAUAGAAAGAAAGAGAGGCCGUCGGC GGCCAUGGAAGAGAGAGAGAGAGAGAGAGAUGAAAUGAUGAUGAUGAUACAGCU GCCGCUCGCGUGCUCACUUCUCUUUCUGUCAGCUCUCCCUG, an RNA fragment encoding a hairpin motif, the fragment arising from O. sativa;

SEQ ID NO: 782 is CGCGGCUGGCUGACAGAAGAGAGUGAGCACACAGCGGGCAGACUGCAUCUGAAA UAAACUGGUGACGACGAAGAAGACGACGGACGCAGCUUGCCGUUGCGUGCUCAC UUCUCUCUCUGUCAGCUCUCUCUG, an RNA fragment encoding a hairpin motif, the fragment arising from O. sativa;

SEQ ID NO: 783 is GCGAGAUUGUUGACAGAAGAGAGUGAGCACACGGCGCGGCGGCUAGCCAUCGGC GGGAUGCCUGCCCCCGCCGCGUGCUCGCUCCUCUUUCUGUCAGCAUCUCUCA, an RNA fragment encoding a hairpin motif, the fragment arising from O. sativa;

SEQ ID NO: 784 is CGCUGGGCGGUGACAGAAGAGAGUGAGCACACGGCCGGGCGGAACGGCACCGGC GGAUGUGCCGUCGCGGCCGCGUGCUCACUGCUCUGUCUGUCAUCCACUCCUC, an RNA fragment encoding a hairpin motif, the fragment arising from O. sativa;

SEQ ID NO: 785 is GCGAGAUUGUUGACAGAAGAGAGUGAGCACACGGCGCGGCGGCUAGCCAUCGGC GGGAUGCCUGCCCCCGCCGCGUGCUCGCUCCUCUUUCUGUCAGCAUCUCUCA, an RNA fragment encoding a hairpin motif, the fragment arising from O. sativa;

SEQ ID NO: 786 is UUGAGAGUGAUGACAGAAGAGAGAGAGCACAACCCGACAGCAGCGACGACGGCG GUCGCUUCUGCCAGGGCCGUGUGCUCUCUGAUCUAUCUGUCAUCGCCGUCCA, an RNA fragment encoding a hairpin motif, the fragment arising from O. sativa;

SEQ ID NO:787is GCUAGGGAGCCGACAGAAGAGAGUGAGCAUAUAUAGUUCUUUCCUUGCAUAUGU GGUCAUAUGUGUGUUGACUGAAGAGAUACAUAUAUAUAGAGAGAGAGAGUUCAU GUGCUUGAAGCUAUAUGUGCUCACUUCUCUUUCUGUCAGCAAAUUAUC, an RNA fragment encoding a hairpin motif, the fragment arising from O. sativa;

SEQ ID NO: 788 is GUUGUGGACGUUGAGCUCCUUUCGGUCCAAAAAGGGGUGUUGCUGUGGGUCGAU UGAGCUGCUGGGUCAUGGAUCCCGUUAGCCUACUCCAUGUUCAUCAUUCAGCUC GAGAUCUGAAAGAAACUACUCCAAUUUAUACUAAUAGUAUGUGUGUAGAUAGGA AAAUGAUGGAGUACUCGUUGUUGGGAUAGGCUUAUGGCUUGCAUGCCCCAGGAG CUGCAUCAACCCUACAUGGACCCUCUUUGGAUUGAAGGGAGCUCUGCAUCUUUG GU, an RNA fragment encoding a hairpin motif, the fragment arising from O. sativa;

SEQ ID NO: 789 is GGUUAUGAAGUGGAGCUCCUUUCGUUCCAAUGAAAGGUUUAUCUGAAGGGUGAU ACAGCUGCUUGUUCAUGGUUCCCACUAUUCUAUCUCAUAGGAAAAGAGAUAGGC UUGUGGUUUGCAUGACCAAGGAGCCGAAUCAACUCCUUGCUGACCACUCUUUGG AUUGAAGGGAGCUCUGCAUCUUGAUC, an RNA fragment encoding a hairpin motif, the fragment arising from O. sativa;

SEQ ID NO: 790 is GAGGAGGAAGAGGAGCUCCUUUCGAUCCAAUUCAGGAGAGGAAGUGGUAGGAUG CAGCUGCCGAUUCAUGGAUACCUCUGGAGUGCGUGGCAGCAAUGCUGUAGGCCU GCACUUGCAUGGGUUUGCAUGACCCGGGAGAUGAACCCACCAUUGUCUUCCUCU AUUGAUUGGAUUGAAGGGAGCUCCACAUCUCUCUC, an RNA fragment encoding a hairpin motif, the fragment arising from O. sativa;

SEQ ID NO: 791 is UGAUGUGAGGAGGAGCUCCUUUCGAUCCAAUUCAGGAGAGGAAGUGGUGGGAUG CAGCUGCCGGUUCAUGGAUACCUCUGCAGUUCAUGCCGGUAGGCCUGCACUUGCA UGGGUUUGCAUGACCUGGGAGAUGAACCUGCCAUUGUGUUCCUCUAUUGAUUGG AUUGAAGGGAGCUCCGGCUACACCUA, an RNA fragment encoding a hairpin motif, the fragment arising from O. sativa;

SEQ ID NO: 792 is GAUGAAGAAGAAGAGCUCCCUUUCGAUCCAAUUCAGGAGAGGAAGUGGUAGGAU GCAGCUGCCGGUUCAUGGAUACCUCUGGAGUGCAGGGCAAAUAGUCCUACCCUU UCAUGGGUUUGCAUGACUCGGGAGAUGAACCCGCCAUUGUCUUCCUCUAUUGAU UGGAUUGAAGGGAGCUCCUCUAGCUACAU, an RNA fragment encoding a hairpin motif, the fragment arising from O. sativa;

SEQ ID NO: 793 is GAAGAAGAAGACGAGCUCCCUUCGAUCCAAUCCAGGAGAGGAAGUGGUAGGAUG CAGCUGCCGGUUCAUGGAUACCUCUGCAGUGCAUGUCGUAGGCUUGCACUUGCA UGGGUUUGCAUGACCCGGGAGAUGAACCCACCAUUGUCUUCCUCUUAUGCUUGG AUUGAAGGGAGCUCUACACCUCUCUC, an RNA fragment encoding a hairpin motif, the fragment arising from O. sativa;

SEQ ID NO: 794 is UGUGUAAGAAGAGAGCUCUCUUCAGUCCACUCUCAGAUGGCUGUAGGGUUUUAU UAGCUGCCGAAUCAUCCAUUCACCUACCAAGAAAGUUGCAGGAGUGUAUCUCUU GGUAGCGGACUGGAUGACGCGGGAGCUAAAAUUUAGCUCUGCGCCGUUUGUGGU UGGACUGAAGGGUGCUCCCUUGCUCAAGC, an RNA fragment encoding a hairpin motif, the fragment arising from O. sativa;

SEQ ID NO: 795 is GAUGGAUGGAAGAGAGCGUCCUUCAGUCCACUCAUGGGCGGUGCUAGGGUCGAA UUAGCUGCCGACUCAUUCACCCACAUGCCAAGCAAGAAACGCUUGAGAUAGCGA AGCUUAGCAGAUGAGUGAAUGAAGCGGGAGGUAACGUUCCGAUCUCGCGCCGUC UUUGCUUGGACUGAAGGGUGCUCCCUCCUCCUCGA, an RNA fragment encoding a hairpin motif, the fragment arising from O. sativa;

SEQ ID NO: 796 is GUGUAGUGUGUGCCUGGCUCCCUGUAUGCCACACAUGUAGACCAACCCAUGGUG UCUGGUUGCCUACUGGGUGGCGUGCAAGGAGCCAAGCAUGCAUGCCUG, an RNA fragment encoding a hairpin motif, the fragment arising from O. sativa;

SEQ ID NO: 797 is CUUGAGAGCGUGCCUGGCUCCCUGUAUGCCACUCAUGUAGCCCAAUCCAUGGUGU GUUUGGAUGCUGUGGGUGGCGUGCAAGGAGCCAAGCAUGCGUGCCAU, an RNA fragment encoding a hairpin motif, the fragment arising from O. sativa;

SEQ ID NO: 798 is AUUGGGAAUGUGCCUGGCUCCCUGUAUGCCACUCAUCUAGAGCAACAAACUUCU GCGAGAGGUUGCCUAUGAUGGAUGGCGUGCACGGAGCCAAGCAUAUUCCCUCC, an RNA fragment encoding a hairpin motif, the fragment arising from O. sativa;

SEQ ID NO: 799 is AAAGGGGAUAUGCCUGGCUCCCUGUAUGCCACUCGCGUAGCUGCCAAACUCAGU UGAAACAACUGCCUUCUCCCGGCGAGAUUCAGGCAUUGUGUUCGUACGUUUGGC UCUACUGCGGAUGGCGUGCGAGGAGCCAAGCAUGACCGUCUC, an RNA fragment encoding a hairpin motif, the fragment arising from O. sativa;

SEQ ID NO: 800 is GUAGGGGAUAUGCCUGGCUCCCUGUAUGCCGCUCGCAUGGCUGCCAACCCAAUGA ACUCGAUCUCGUUGUUGGCCGCUGCGUACGGCGUGCGAGGUGCCAAGCAUGGCCC UCUU, an RNA fragment encoding a hairpin motif, the fragment arising from O. sativa;

SEQ ID NO: 801 is GGAUUAACGCUGCCUGGCUCCCUGAAUGCCAUCCGAGAAGCGUGCCGCUGUGGCC GGCUGCUUCCUGGUUGGCAUUGAGGGAGUCAUGCAGGGUUUGCUC, an RNA fragment encoding a hairpin motif, the fragment arising from O. sativa;

SEQ ID NO: 802 is GUGGUGAUGCCUGGGCGCAGUGGUUUAUCGAUCCCUUCCCUGCCUUGUGGCGCU GAUCCAGGAGCGGCGAAUUUCUUUGAGAGGGUGUUCUUUUUUUUUUCUUCCUUU UGGUCCUUGUUGCAGCCAACGACAACGCGGGAAUCGAUCGAUAAACCUCUGCAU CCAGUUCUCGCCUU, an RNA fragment encoding a hairpin motif, the fragment arising from O. sativa;

SEQ ID NO: 803 is UGGGUGAUGCCUGGGCGCAGUGGUUUAUCGAUCUCUUCCCUGCCUUGUGCUGCU CCGAUCGAUGCCCGUGCUGAUUCUUGAUAAUAUACAACGCAGGAAUCGAUCGAU AAGCCUCUGCAUCCAGAUCUCACUUU, an RNA fragment encoding a hairpin motif, the fragment arising from O. sativa;

SEQ ID NO: 804 is CCGUGCACGGUGGAGAAGCAGGGCACGUGCAUUACCAUCCACUCGCCUGCCGGCC GCCGGCCGCCAUUGCCAUGGAUGGUUCUUCAUGUGCCCGUCUUCUCCACCGAGCA CUA, an RNA fragment encoding a hairpin motif, the fragment arising from O. sativa;

SEQ ID NO: 805 is AGGACCGCGUUGGAGAAGCAGGGCACGUGCAUGCAUAUGUUCAUCAUCAUCUUC UUCCUCCUCCUCUAGCUCCAGCCUUGUGUGGGUUGGAAGUUUAGAUAGAACUCG CACUGCACGUGGUCUCCUUCUCCAUCCCGGUCUU, an RNA fragment encoding a hairpin motif, the fragment arising from O. sativa;

SEQ ID NO: 806 is AGGUUCUUGUUGGAGAAGCAGGGUACGUGCAAAAUGCACACCGGUUGGUCGAGC UAAUUAACAAGCUCUGACGACCAUGGUGAUCGAAUGCACGUGCUCCCCUUCUCCA CCAUGGCCUU, an RNA fragment encoding a hairpin motif, the fragment arising from 0. sativa;

SEQ ID NO: 807 is CAAACCGUGCUGGAGAAGCAGGGCACGUGCUCGACGGCGGGGCUGGCUGGCCGG CCGGCUUGCAGCAUGUGCGCUCCUUCUCCAGCAUGGCUUC, an RNA fragment encoding a hairpin motif, the fragment arising from O. sativa;

SEQ ID NO: 808 is UUGUGCAGGGUGGAGAAGCAGGGCACGUGAGCGGCCAUCCAGUGUAGCUUCGCU GCGCGUCCAUGGCGGCGAACGCGCGUGAUCUGGAGUUUGGAUGGUCGUUCAUGU GUCCGUCUUCUCCACCGAGCACUG, an RNA fragment encoding a hairpin motif, the fragment arising from O. sativa;

SEQ ID NO: 809 is UUGCUUCUGAGUGGAAUGUUGUCUGGUUCAAGGUCUCAUACACCUUGUGGUUUU GAGGAUGAUUUGUGCAAGGUUUUUCAUUCCUCUCAUCCGUGGGAUCUCGGACCA GGCUUCAUUCCCCUCAGAGAUAG, an RNA fragment encoding a hairpin motif, the fragment arising from O. sativa;

SEQ ID NO: 810 is UUCAUUUUGAGGGGAAUGUUGUCUGGCUCGGGGCUACUUUUAAUUUCUCUCUCU UUUGAUAUCUUCUUUUCUCGAUCUCCUAGCUUGAUCUUUUUGAUCUCUCAAAUC GAUCUUAAGAAAAAGAUCAGUCAAAGAGAUGAGAGUAGAUGUCUGUAGAUCUCG GACCAGGCUUCAUUCCCCCCAAACAGAA, an RNA fragment encoding a hairpin motif, the fragment arising from O. sativa;

SEQ ID NO: 811 is UGGCAGUUGAGGGGAAUGUUGUCUGGUCCGAGACCUAACACCGGGCGGAAUGGC GGAUUCAGCUGCAGCUAAGCAAGCUAGGUGGGGGGUUUCGGACCAGGCUUCAUU CCCCCCAACUCAAC, an RNA fragment encoding a hairpin motif, the fragment arising from O. sativa;

SEQ ID NO: 812 is UUCACUUUGAGGGGAAUGUUGUCUGGCUCGAGGUGCAUGGAGAAACCUCUGAUC GAUCAGGUUUGAUCUGUAGAGACUGAUCUCGGACCAGGCUUCAUUCCCCUCAAG UAAAG, an RNA fragment encoding a hairpin motif, the fragment arising from O. sativa;

SEQ ID NO: 813 is UUGUUUUUGGGUGGAAUGUUGUCUGGUUCAAGGCCCCUUAGGAUGUGUGAUUUU UGAUGGUUUAUGCAUUCAUCUUGAUGCGAACAUCUAUCUCGGAUCUUUGGGUUC UCGGACCAGGCUUCAUUCCCCUCAGAGAUAG, an RNA fragment encoding a hairpin motif, the fragment arising from O. sativa;

SEQ ID NO: 814 is AUCUUGUUGAGAGGAAUGUUGUCUGGCCUGAGAUCGUACCAUAGUGGUGGGUAC ACGUGGACGGUCUCGGACCAGGCUUCAUUCCCCUCAACAACUC, an RNA fragment encoding a hairpin motif, the fragment arising from O. sativa;

SEQ ID NO: 815 is AGCAUGGUGUCUGGAAUGGAGGCUGAUCCAAGAUCCUUGCUUGGUGCAAAAUAC UAGGGCAUUGUUGUAAGUGCCAUUAGUUCUUUUUUGUUUCCGAGUUUGUUAUCG AGGAUCUCGGACCAGGCUUCAUUCCUCACACCGUGCU, an RNA fragment encoding a hairpin motif, the fragment arising from O. sativa;

SEQ ID NO: 816 is GGUGGCUUGUGGGGAAUGUUGGCUGGCUCGAGGCAUCCACAUCUUAAUUCCUCU CCGGCGAUCGAGCCGGCUCGGGCGUGUGGAGGCGUCGGACCAGGCUUCAUUCCUC GCAAGCCGAU, an RNA fragment encoding a hairpin motif, the fragment arising from 0. sativa;

SEQ ID NO: 817 is AGAUAGGUGUUUGGAAUGCAGUUUGAUCCAAGAUCUGCCUAUAUAUAUGGUGUG UAUAUCAUAUCUUGUGAUAUGGGGGAUAUGCAACAAGUGUGUGACAGGGGUGGG UAGAUCUCGGAUCAGGCUUCAUUCCUCACACCAAUAC, an RNA fragment encoding a hairpin motif, the fragment arising from O. sativa;

SEQ ID NO: 818 is AGAUAGGUGUUUGGAAUGCAGUUUGAUCCAAGAUCUGCCUAUAUAUAUGGUGUG UAUAUCAUAUCUUGUGAUAUGGGGGAUAUGCAACAAGUGUGUGACAGGGGUGGG UAGAUCUCGGAUCAGGCUUCAUUCCUCACACC, an RNA fragment encoding a hairpin motif, the fragment arising from O. sativa;

SEQ ID NO: 819 is AUUAGGUUAAGGGGUUUGUUGUCUGGCUCGAGGCAUCCGGGACUCCGGUUUCUC CUUACCUACUGGAGGCGCCUAGCUUCCGGCGAGCUCGGAGCCUCGGACCAGGCUU CAAUCCCUUUAACCAUGC, an RNA fragment encoding a hairpin motif, the fragment arising from O. sativa;

SEQ ID NO: 820 is GUUAGGUUAAGGGGAUUGUUGUCUGGUUCAAGGUCUCCACAUUGUGCAAAAUGU UCAUUCAUGGAGGCACAGGAUGCUUGGUGAUCUCGGACCAGGCUUCAAUCCCUU UAACCAGCA, an RNA fragment encoding a hairpin motif, the fragment arising from O. sativa;

SEQ ID NO: 821 is UGUGAAUGAGUGAAGCUGCCAGCAUGAUCUAGCUCUGAUUAAUCGGCACUGUUG GCGUACAGUCGAUUGACUAAUCGUCAGAUCUGUGUGUGUAAAUCACUGUUAGAU CAUGCAUGACAGCCUCAUUUCUUCACA, an RNA fragment encoding a hairpin motif, the fragment arising from O. sativa;

SEQ ID NO: 822 is AGAGAAAGCGUGAAGCUGCCAGCAUGAUCUAACUUGCAGACAAGAAAUCAGCUC AGCUCGCUGGUUUCGAACAGGAAGGCGGCUAGCUGAGGCUUCUUCUGAGUACGU GAUGGUUAGAUCAUGCUGUGACAGUUUCACUCCUUCCCU, an RNA fragment encoding a hairpin motif, the fragment arising from O. sativa;

SEQ ID NO: 823 is AGGGAACGAGUGAAGCUGCCAGCAUGAUCUAGCUCUGAAUGAUCAACAAGAUGU GCUCCCACACUGCCUUCCUGUGGAUCUUGAGCUGUUGCUAGUCUUGUGGUCAUG CCUUGCUAGGUCAUGCUGCGGCAGCCUCACUUCUUCCCA, an RNA fragment encoding a hairpin motif, the fragment arising from O. saliva;

SEQ ID NO: 824 is CAUUAGGAGCUGAAGCUGCCAGCAUGAUCUGAUGAGUGCUUAUUAGGUGAGGGC AGAAUUGACUGCCAAAACAAAGAUCAGAUCAUGCUGUGCAGUUUCAUCUGCUUG UG, an RNA fragment encoding a hairpin motif, the fragment arising from O. saliva;

SEQ ID NO: 825 is UGUGAGAGAAUGAAGCUGCCAGCAUGAUCUGGUUGUCAGGCAUGAGCCAAAUCU AUCCAUGGUGUUGGUGGUACUGAAAUUACCGCGUUUUCGAGGUUUUUCGUCGUG UCAACUUGCGAAGGGAAUUACGGGUUCUUGAUGAGCAUUGGUGAUAGGAGGUGU GGGCUUGGUUAGUAGAGGUAGAAUUAUGAUUGUUCUUGUGAGUUUCAGUAAGA GGUGGGAGUGAUUGGAAUUUGGCUCCAUCAGAUCAUGUUGCAGCUUCACUCUCU CACC, an RNA fragment encoding a hairpin motif, the fragment arising from O. saliva;

SEQ ID NO: 826 is CACAAGUGGAUGAAGCUGCCAGCAUGAUCUGAUCACAGUAGUUCUCUAGCUGAU GAUGAUUUACAAAACCUAGAGACAUGCAUCAGAUCAUCUGGCAGUUUCAUCUUC UCAUG, an RNA fragment encoding a hairpin motif, the fragment arising from O. saliva;

SEQ ID NO: 827 is CAUAAGCAGGUGAAGCUGCCAGCAUGAUCUGAAAGCAUCUCAAACCAGCGAUCA GAUCAUCCGGCAGCUUCAUCUUCUCAUG, an RNA fragment encoding a hairpin motif, the fragment arising from O. saliva;

SEQ ID NO: 828 is CACAAGUUGGUGAAGCUGCCAGCAUGAUCUGAUGAUGAUGAUGAUCCACCUCUC UCAUCUGUGUUCUUGAUUAAUUACGGAUCAAUCGAUCAGGUCAUGCUGUAGUUU CAUCUGCUGGUU, an RNA fragment encoding a hairpin motif, the fragment arising from O. saliva;

SEQ ID NO: 829 is UGUGAGAGGCUGAAGCUGCCAGCAUGAUCUGGUCCAUGAGUUGCACUGCUGAAU AUAUUGAAUUCAGCCAGGAGCUGCUACUGCAGUUCUGAUCUCGAUCUGCAUUCG UUGUUCUGAGCUAUGUAUGGAUUUGAUCGGUUUGAAGGCAUCCAUGUCUUUAAU UUCAUCGAUCAGAUCAUGUUGCAGCUUCACUCUCUCACU, an RNA fragment encoding a hairpin motif, the fragment arising from O. saliva;

SEQ ID NO: 830 is CGCCUCGGGCUCGCUUGGUGCAGAUCGGGACCCGCCGCCGCCGCUGCCGGGGCCG GAUCCCGCCUUGCACCAAGUGAAUCGGAGCCG, an RNA fragment encoding a hairpin motif, the fragment arising from O. sativa;

SEQ ID NO: 831 is UGGUCUUGUGAGGCUUGGUGCAGCUCGGGAACUGUUCUUGAUGGACUGGCAGGA ACUCCAUGUCCACCACUGCCACUCCUGUGUUGUGGCAUUCCUCCUUGCCGUU, an RNA fragment encoding a hairpin motif, the fragment arising from O. sativa;

SEQ ID NO: 832 is GGCCAUGGUGCAGCCAAGGAUGACUUGCCGAUCGAUCGAUCUAUCUAUGAAGCU AAGCUAGCUGGCCAUGGAUCCAUCCAUCAAUUGGCAAGUUGUUCUUGGCUACAU CUUGGCC, an RNA fragment encoding a hairpin motif, the fragment arising from O. sativa;

SEQ ID NO: 833 is GAACGGAAUGCAGCCAAGGAUGACUUGCCGGUACGUGUAUGCAUGUUUCAAGGU ACUAUAUGUGCCCCCAACUGUUUUAGAUCCAUGCUGACAUUUUCCGGCAAGUUG UCCUUGGCUACGUCUUGUUC, an RNA fragment encoding a hairpin motif, the fragment arising from O. sativa;

SEQ ID NO: 834 is GAACGGGAUGCAGCCAAGGAUGACUUGCCGGCUCCUGGUAUUGGGGGAAUCUCA GCUUUGCUGAAGCGCCUUGGAGUUAGCCGGCAAGUCUGUCCUUGGCUACACCUA GCUC, an RNA fragment encoding a hairpin motif, the fragment arising from O. sativa;

SEQ ID NO: 835 is AUUUAUCGUGUAGCCAAGGAUGAAUUGCCGGCGUUUCACGCUGUUGAUGGUGCG UGCAUAUAUAAGUUGGCGCCGGCAAGUCAUUUCAGGCUACAUGUUUGCC, an RNA fragment encoding a hairpin motif, the fragment arising from O. sativa;

SEQ ID NO: 836 is GCUGGUUGUGUAGCCAAGGAUGACUUGCCGGCCUGAUUUGUGUUCAUCAGCAAU CCAGCAUAUGCUGUAUUGCCGUGUGUGAUCGAUCGAUGCAUGGACCGGCAAGUU AUUUUCUUUGGCUACAUUACAACC, an RNA fragment encoding a hairpin motif, the fragment arising from O. sativa;

SEQ ID NO: 837 is GCUGAUUCGGUAGCCAAGGAUGACUUGCCUAAUGCCUAUGUGCAUGUGUUUAUA CGCUGCUCAUCUGCAUUUUGAUUAUCCCCUGAUCAGUCCUGUCGUCAAUAUAUG UGUGUGUAGUACUCUGUACUCAUACAUAUAUAGGCAUGUCUUCCUUGGCUAUUC GGAGCGG, an RNA fragment encoding a hairpin motif, the fragment arising from O. sativa;

SEQ ID NO: 838 is CUGCCUCUGGUAGCCAAGGAUGACUUGCCUAUUGUGCUCUUCUGAAUGAUGCAG UGCCAUGAUCAGUGUGGCCUGGCUGGUUCAGAUGAGCCGAGAUAGGCAGUCUCC UUGGCUAGCCUGAGUGGC, an RNA fragment encoding a hairpin motif, the fragment arising from O. sativa;

SEQ ID NO: 839 is UUAGCUCUGGUAGCCAAGGAUGACUUGCCUGUGUCCUUGUGUGUAAGGAUCAUU AAUUAUUAUUCAGAAAAUGAUCCUUUCAGCAGGUUUCAUGGGCAGUCUCCUUGG CUAGCCUGAGUGAU, an RNA fragment encoding a hairpin motif, the fragment arising from O. sativa;

SEQ ID NO: 840 is GUAGCUCUGGUAGCCAAGGAUGACUUGCCUGUGUCCUUGUGUAGAGGAUCAUUC AGAAAAUGAGCCUUGAACUGGUUCAUAGGCAGUCUCCUUGGCUAGUCUGAGUCG, an RNA fragment encoding a hairpin motif, the fragment arising from O. sativa;

SEQ ID NO: 841 is UCGCAUCUGGUAGCCAAGGAUGACUUGCCUGUGUCUCUGCUCAUGUGCAGUAGA AGAAGAUGCAUUUCUAGCUGCUUUCUGCAUAUGUGAUCUCACAGGCAGUCUCCU UGGCUAGCCUGAGCGGC, an RNA fragment encoding a hairpin motif, the fragment arising from O. sativa;

SEQ ID NO: 842 is UCUGUCUAGAUAGCCAAGGAUGACUUGCCUGUGGCCUCUUGGAGAGAGAGGUOU AGCUUAAUUAGCAGCAUGGUUUGAGCAUUGCUUGAUCGGUUGAUCGCUUCGCUU GCUCUGCAUGAGAUCUUACAGGCAGUCUCCUUGGCUAGUCUGGGCGGC, an RNA fragment encoding a hairpin motif, the fragment arising from O. sativa;

SEQ ID NO: 843 is UUAUCUCUGAUAGCCAAGGAUGACUUGCCUGUGUCCUCCCUGAAGGAUUAGCAA UUUAAUGAUCCUUUAAGCUGGUUCAUGGGCAGUCUCCUUGGCUAGCCUGAGUGG, an RNA fragment encoding a hairpin motif, the fragment arising from O. sativa;

SEQ ID NO: 844 is UGAGUCCUGGUAGCCAAGGAUGACUUGCCUGUAUAUCUAUAUAUAUAUGUGUGU GUGAUCAAUGGAUGGAUUGAUCAAGCUGCUUGCAGGCUCAUGCAUAUAUAUGUA CAGGCAGUCUCCUUGGCUAGCCCGGCUACC, an RNA fragment encoding a hairpin motif, the fragment arising from O. sativa;

SEQ ID NO: 845 is CUCCCUUUGGUAGCCAAGAAUGACUUGCCUAUGCGUUUUGCCUUGUGUUGGCUC AUCCAUCCGUCUAUCAGCCGUUGCAGAUUUGCAGUGACAGAUUAAAGGGUUUCU GAAAGAAAUUCUUGUGAUGGAUGUGCAAUGUGGCUGCAUGGGCCGGUCUUCUUG GCUAGCCAGAGUGGC, an RNA fragment encoding a hairpin motif, the fragment arising from O. sativa;

SEQ ID NO: 846 is CUCCCUUUGGUAGCCAAGAAUGACUUGCCUAUGCUUUUGCCCUCUGUUGGCUCA UCCAUCCAUCUAUCUAUCUGCCAUGGCAGAUUAAGGGUUUUUGAAAGAAAUUCU UGUGAUAGGAUGUGCAAUGUGGCUGCAUGGGCCGGUCUUCUUGGCUAGCCAGAG UGGC, an RNA fragment encoding a hairpin motif, the fragment arising from O. sativa;

SEQ ID NO: 847 is GAGCAAGGUGUAGCCAAGGACAAACUUGCCGGAUCAACAGAGAAGGACUGCCAG UCUCCGGCCAAUUAAUUAACCUCGCCGUCGGCCAUCGCCGGCCGGCAAGUCAUCC UUGGCUGCAUCCUGCUC, an RNA fragment encoding a hairpin motif, the fragment arising from O. sativa;

SEQ ID NO: 848 is CCACUCAGGCUAGCCAAGGAGACUGCCCAUGAACCAGCUUAAAGGAUCAUUAAA UUGCUAAUCCUUCAGGGAGGACACAGGCAAGUCAUCCUUGGCUAUCAGAGAUAA, an RNA fragment encoding a hairpin motif, the fragment arising from O. sativa;

SEQ ID NO: 849 is UGGAAAGAGCGAUAUUGGUGAGGUUCAAUCCGAUGAUUGGUUUUACAGCAGUGG UAAAAUCAGUAUCUGAUUGAGCCGCGCCAAUAUCUCUUCCUCUC, an RNA fragment encoding a hairpin motif, the fragment arising from O. sativa;

SEQ ID NO: 850 is GCGACGACGGGAUAUUGGGGCGGUUCAAUCAGAAAGCUUGUGCUCCAGAAGCGA GGAGCUCUACUCUUUUGAUUGAGCCGUGCCAAUAUCACGUCGCAUC, an RNA fragment encoding a hairpin motif, the fragment arising from O. sativa;

SEQ ID NO: 851 is GUGGGAACGGGAUAUUGGUGCGGUUCAAUCAGAAAGCUUGUGCUCCGAAGGCGA GGGGCUCCACUCUUUGAUUGAGCCGUGCCAAUAUCACGUCGCCUU, an RNA fragment encoding a hairpin motif, the fragment arising from O. sativa;

SEQ ID NO: 852 is UUGUAGCUAUGAUGUUGGCCCGGCUCACUCAGAUGGAUCAUCGGUGCAGAAGAG UGCAUGAAUCUGAUGCAGUCUCAGUGUAGUAUGCUCCAUGCUGGAACUUCUGAU UGAGCCGUGCCAAUAUCUCAGCACCAU, an RNA fragment encoding a hairpin motif, the fragment arising from O. sativa;

SEQ ID NO: 853 is UGGUAGCUAUGAUGUUGGCUCGGCUCACUCAGACGGCAUUGGCGUGAUGCAAAG CAUGCAUGCGUGCUCGCUAGCUCACUUGUGUUUCUGAUUGAGCCGUGCCAAUAU CUUAGUGCUCU, an RNA fragment encoding a hairpin motif, the fragment arising from O. sativa;

SEQ ID NO: 854 is GGGAGAGUGCGAUGUUGGCAUGGUUCAAUCAAACCGGGCAAACUUAUGCACUAG CUAAGCAAGAUGCAGGGAUACGCAGUAUGGUUUUGUUUGGUCUGAUUGAGCCGU GCCAAUAUCACAAGCUUGC, an RNA fragment encoding a hairpin motif, the fragment arising from O. sativa;

SEQ ID NO: 855 is GACAUGGCAUGGUAUUGACUUGGCUCAUCUCAGCAACAGCAAACUGCAUGCAGC GCUGGAGGUGAGCCGAGCCAAUAUCACUUCAUGUC, an RNA fragment encoding a hairpin motif, the fragment arising from O. sativa;

SEQ ID NO:856is GUGUUUGCGGGCGUGGCAUCAUCAAGAUUCACAUCCAUGCAUAUAUCACAAGAC GCACAUAUACAUCCGAUUUGGCUGAGAAUCUUGAUGAUGCUGCAUCCGCAGACA A, an RNA fragment encoding a hairpin motif, the fragment arising from O. sativa;

SEQ ID NO: 857 is GUGAUUUCUGACGUGGCAUCAUCAAGAUUCACACAUUACAUUGCAUGCAUGCAU AUGUCUAUGCAUCUUUGAGCUUGUUGUUCUGAUCUCAACAACCUAGCUAGCUAA UAUUUCUCUCCUGGCCCUGACCUGCAUGAUGCAUGGUUGCACGCAUGGAGAGAG AAGAGAGAGAUCGAAGCUAAUUAAGCGCAUGUGUAUAUAUGUGUGGGAAUCUUG AUGAUGCUGCAUCGGAAAUUAA, an RNA fragment encoding a hairpin motif, the fragment arising from O. sativa;

SEQ ID NO: 858 is CUUGUUGCGGGUGCAGCGUCAUCAAGAUUCACGUGUGCCGCACGGCACACGUAU CGGUUUUCAAGUGUAGUCAUCGUGCGUGAAUCUUGAUGAUGCUGCACCAGCAAA GAG, an RNA fragment encoding a hairpin motif, the fragment arising from O. sativa;

SEQ ID NO: 859 is UGGGGAAGCAUCCAAAGGGAUCGCAUUGAUCCUUCAUCGCUCUCGCUCGCUUCCA UGGCGGUCGUCGUCUACAAGCAGCUUGACGGAUCAUGCGAUCCUUUUGGAGGCU UCCUCU, an RNA fragment encoding a hairpin motif, the fragment arising from O. sativa;

SEQ ID NO: 860 is UACUGAGAGUUCUUUGGCAUUCUGUCCACCUCCUUGUCGAAUCCUCAGAGACAG AAAUCUCAUAUCUGUUGAUCUUGGAGGUGGGCAUACUGCCAAUGGAGCUGUGUA GG, an RNA fragment encoding a hairpin motif, the fragment arising from O. sativa;

SEQ ID NO: 861 is UUGUCCACUGGAGUUCUCCUCAAUCCACUUCAGUAGAUAGCUAUGGCUAGGCCU CAUUGCAUUGCACUGUUACAUAACUGUGAUCAUGGGGCCAAAAGCUAGCUAUGU AUAGUGAAGUGCUUGGGGGAACUCCAGUUGACAC, an RNA fragment encoding a hairpin motif, the fragment arising from O. sativa;

SEQ ID NO: 862 is GAGUCCCUAGGAGUUCCUUUCAAGCACUUUACGACACACUGUAUUGAGAGUUGU CGUGAAGUGUUUGGGGGAACUCUUAGUGUCGC, an RNA fragment encoding a hairpin motif, the fragment arising from O. sativa;

SEQ ID NO: 863 is GUAUUAUCAAGAGUUCUCUUUAAGCACUUCAUACGACACCAUUAUUUAUAGGGU UGUUGUGAAGUGUUUGGAGGAACUCUCGGUGUCUU, an RNA fragment encoding a hairpin motif, the fragment arising from O. sativa;

SEQ ID NO: 864 is GUAUUGUCGUGAGUUCCCUUCAAGCACUUCACGUGGCACUAUCUCAAUGCCUAC UAUGUGAAGUGUUUGGGGGAACUCUCGGUAUCAC, an RNA fragment encoding a hairpin motif, the fragment arising from O. sativa;

SEQ ID NO: 865 is GUAUUAUCGAGAGUUCCCUUCAACCACUUCACGUGGCACUGUUUCAAGGCCUAU UGUGUGAAGUGUUUGGGGGAACUCUCGAUAUCAC, an RNA fragment encoding a hairpin motif, the fragment arising from O. sativa;

SEQ ID NO: 866 is GUAUUAUCGCGGGUUCCCUUCAAUCACUUCACAUGGUACUAUUUCAAGGCCUAC UAUGUGAAGUAUUUGGGGGAACUCUCGAUGUCAC, an RNA fragment encoding a hairpin motif, the fragment arising from O. sativa;

SEQ ID NO: 867 is GUAUCACCGUGAGUUCCCUUCGAACACUUCACGUGGCACUAUUUCAAUGCCUAU UGUGAAGUGUUUGGGGGAACUCUCGAUGUCAU, an RNA fragment encoding a hairpin motif, the fragment arising from O. sativa;

SEQ ID NO: 868 is UUGUUACCUGGAGUUUCCUCAACACACUUCACAUCUGCUAGGCCCUAUUACAAU UGCGCAAUGUGGGGUCUGCAAUUGGUAGUGAAGUGUUUGGGGGAACUCUAGGUG GCAC, an RNA fragment encoding a hairpin motif, the fragment arising from O. sativa;

SEQ ID NO: 869 is GUUUUACCGGGAGUUCCCUACAAGCACUUCACGUAGAGCUUUCUAUUGACAUGG AGCUUUAGAACAAUGUGAAGUGUUUGGGGGAACUCUUGGUACCAA, an RNA fragment encoding a hairpin motif, the fragment arising from O. sativa;

SEQ ID NO: 870 is GUGUUCCCAAGAGUUCCUUGCAAGCACUUCACAUAGAACUUCUGUUACUCUCAU GUAACAUUGGGAACUUGAGAAGCUACUGUGAAGUGUUUGGGGGAACUCUAGGUG GCAC, an RNA fragment encoding a hairpin motif, the fragment arising from O. sativa;

SEQ ID NO: 871 is GUUUUAUCGGGAGUUUCCUUCAAGCACUUCACGUAGAGCUUUCUAUUGAUAUGG AGCUUUGGAACAAUGUGAAGUGUUUGGGGGAACUCUUGAUACCAA, an RNA fragment encoding a hairpin motif, the fragment arising from O. sativa;

SEQ ID NO: 872 is GUGGCCCCAGGAGUUCCUUGCAAGCACUUCACAUAGAACUUCAGUUACUCUCAU ACAACAUUGUGAUUUUGAGAAGCUAUUGUGAAGUGUUUGGGGGAACUCUCGGUG CCAA, an RNA fragment encoding a hairpin motif, the fragment arising from O. sativa;

SEQ ID NO: 873 is AUGUCCCUAAGCGUUCCUUCCAAGCACUUCACACAGAGCUUUUAUUUCUCUCACA UCGAUUGAGAACUUAAUUAGAAGCUUUUGUGAAGUGUUUGGGGGAACUCUUGGU GCCAC, an RNA fragment encoding a hairpin motif, the fragment arising from O. sativa;

SEQ ID NO: 874 is GUAUCACCGUGAGUUCCCUUCAAGCACUUCACGUGGCACUAUUUCAAUGCCUAU UGUGAAGUGUUUGGGGGAACUCUCGAUGUUCC, an RNA fragment encoding a hairpin motif, the fragment arising from O. sativa;

SEQ ID NO: 875 is UUAUCCACUGGAGUUCUCCUCAAACCACUUCAGCAGAUAGCUAGCUAGGCCUCAU UGCAUUGCACCACUGUUGCAUAACUAUGAGCAUGGGGCCAAAAGUUAGCUGCVU AUAGUGAAGUGUUUGGGGAAACUCCGGUUGGCAA, an RNA fragment encoding a hairpin motif, the fragment arising from O. sativa;

SEQ ID NO: 876 is ACACUGCCAGGAAUUCCCUUCAAGCAAUUCAUGAAACAAUAUUUUGAGAGUUGU UGUGAAGCGUUUGGGGGAAAUCUCAGUGUCGC, an RNA fragment encoding a hairpin motif, the fragment arising from O. sativa;

SEQ ID NO: 877 is UUAUCCACUGGAGUUCUCCUCAAACCACUUCAGCAGAUAGCUAGCUAGGCCUCAU UGCAUUGCACCACUGUUGCAUAACUAUGAGCAUGGGGCCAAAAGUUAGCUGCUU AUAGUGAAGUGUUUGGGGAAACUCCGGUUGGCAA, an RNA fragment encoding a hairpin motif, the fragment arising from O. sativa;

SEQ ID NO: 878 is UUAUCCACUGGAGUUCUCCUCAAACCACUUCAGCAGAUAGCUAGCUAGGCCUCAU UGCAUUGCACCACUGUUGCAUAACUAUGAGCAUGGGGCCAAAAGUUAGCUGCUU AUAGUGAAGUGUUUGGGGAAACUCCGGUUGGCAA, an RNA fragment encoding a hairpin motif, the fragment arising from O. sativa;

SEQ ID NO: 879 is ACACUGCCAGGAAUUCCCUUCAAGCAAUUCAUGAAACAAUAUUUUGAGAGUUGU UGUGAAGCGUUUGGGGGAAAUCUCAGUGUCGC, an RNA fragment encoding a hairpin motif, the fragment arising from O. sativa;

SEQ ID NO: 880 is CUUUGUGAUCUUCCACAGCUUUCUUGAACUGCACGCAUGAUGAAUAAUCCCUUU GGUUAAUUGUGAUCUGGUCUCUGAGAGAUCGUAGCUAGACUCGAUCGGUUGCAU UGGCAUCAGAGAGAGCAGUUCAAUAAAGCUGUGGGAAAUUGCAGAG, an RNA fragment encoding a hairpin motif, the fragment arising from O. sativa;

SEQ ID NO: 881 is CUUUGUGGUCUUCCACAGCUUUCUUGAACUGCAUCUUUGAGAGAGAUUAGCAUC CCUAUGUGUGGAUUUUGCUUGCACGAGUGUGCAGUUCAAUAAAGCUGUGGGAAA UUACAGAG, an RNA fragment encoding a hairpin motif, the fragment arising from O. sativa;

SEQ ID NO: 882 is UGCCAUGCCUUUCCACAGCUUUCUUGAACUUCUCUUGUGCCUCACUCACUUUCAU UACUGGAGAGAUAUGCAUCAUCAGUGGAAGCUUAUAGGGAGAGGAGUACAAGAA GAGGGUCAAGAAAGCUGUGGGAAGAAAUGGCA, an RNA fragment encoding a hairpin motif, the fragment arising from O. sativa;

SEQ ID NO: 883 is AUCAAAUGCAUCAUUGAGUGCAGCGUUGAUGAACAACGGUAACCGGUCCAUGUU GAUGCGCAUUUGGCCGGUGAUCUGAUCAUCAUCAGCGCUUCACUCAAUCAUGCG UUUGGC, an RNA fragment encoding a hairpin motif, the fragment arising from O. sativa;

SEQ ID NO: 884 is AGGGAAGGCAUUAUUGAGUGCAGCGUUGAUGAACCUGCCGGCCGGCUAAAUUAA UUAGCAAGAAAGUCUGAAACUGGCUCAAAGGUUCACCAGCACUGCACCCAAUCA CGCCUUUGCU, an RNA fragment encoding a hairpin motif, the fragment arising from 0. sativa;

SEQ ID NO: 885 is GCUGAACCCAGAGGAGUGGUACUGAGAACACAGGUGCCAAUACAAUGUAUGGUG AGCUACUGUAUAAUGGAGUAAUUCUGUAACUGUGUUCUCAGGUCACCCCUUUGG GUUUCUU, an RNA fragment encoding a hairpin motif, the fragment arising from O. sativa;

SEQ ID NO: 886 is GGAGUUCCUACAGGGGCGAGCUGGGAACACACGGUGAUGAGGCGGUCUGGUCUU UCGUGUGUUCUCAGGUCGCCCCUGCCGGGACUCU, an RNA fragment encoding a hairpin motif, the fragment arising from O. sativa;

SEQ ID NO:887is CUGUGAAUUACAGGGCAGUUCACCUUUGGCACAAGGGCAAGCAGUAGAAACCAU GCGUGCUUGCUAGAGCUGGAAAUGAUGCUGGUAGCAUUGCAUGGUUCAGGGAUC ACAGAUCUCGUGCCAAAGGAGAAUUGCCCUGCGAUUUUGUC, an RNA fragment encoding a hairpin motif, the fragment arising from O. sativa;

SEQ ID NO: 888 is GUGAGAAUCACAGUGCGAUUCUCCUCUGGCAUGGCAUGAGAGGCCUAAAAAAGA GACGCACUGCCGUGCCAAAGGAGAAUUGCCCUGCCAUUCAGAA, an RNA fragment encoding a hairpin motif, the fragment arising from O. sativa;

SEQ ID NO: 889 is CGGCGAAUUACAGGGCGGUUUCUCCUUUGGCACGUACGGAGGCAAGGCAUGCGG UGAAAAAUCUCUAGCUAGCCAUGCGUGCCAAAGGAGAAUUGCCCUGCGAUUCAC CA, an RNA fragment encoding a hairpin motif, the fragment arising from O. sativa;

SEQ ID NO: 890 is AAGACAGUAGUAGGCAGCUCUCCUCUGGCAGGUGCAUUCUAGGUGAUUUUGUAA UUGUAUAUGCAUCCAAGGUAUAUACAGUCCGGCCAUGGUGCUACAUUGCAAUCA UCCAUAUGUGAUUGCAUUGUGUAUAUAUAUACAUGGUGGCCUUUGAUAGACCAU CAUAUAUCGGUUGGUUAUGUGCAUGUAUGUAUAUACCAGCUGCUACUAGCUUUG AUCGAUCGCCAUGUAGCGAUUGAAUUCACCAAAACGGCCUGCCAAAGGAGAGUU GCCCUGCGACUGUCUU, an RNA fragment encoding a hairpin motif, the fragment arising from O. sativa;

SEQ ID NO: 891 is ACAUGCAUUACCGGGUGAGUCUUCCUUGGCAGUGUUCGAAUCGGCAGUACCGGU CUGCAAGUGAUCGGUCAAUCACCAGUUCACCACUGCCAAAGGAGAUUUGCCCAGC AAUGCAACU, an RNA fragment encoding a hairpin motif, the fragment arising from O. sativa;

SEQ ID NO: 892 is UGGUGGAUUACCGGGCCAUGUCUCCUUGGGCAGAGGUGAUCAGAUUGCACACUU CACUUCAACCUCUUGCUCUAGCUUGUUCUCUCUGCCAAAGGAGAUUUGCCCAGCA AUCCACAU, an RNA fragment encoding a hairpin motif, the fragment arising from O. sativa;

SEQ ID NO: 893 is AUGUGCAUUGCAGGGCAACUACUCCAUUGGCGAGGGAUGGAUUGGAUAUGGAUA UGGCUGAUGCUUCCAUUUGAUCCCAUCCCUAUCUGCCAAAGGAGAUUUGCCCGGC GAUUCACUC, an RNA fragment encoding a hairpin motif, the fragment arising from O. sativa;

SEQ ID NO: 894 is CCAUGCAUUACUGGGCAGGUCUCCCUUGGCAGUGGCCGAUCGAGCUGAUCAAAA CCACGCAAAAGCCACUGCCAAAGGAGACUUGCCCAGCAAUGCAGAU, an RNA fragment encoding a hairpin motif, the fragment arising from O. sativa;

SEQ ID NO: 895 is GUGAGAAUCACAGUGCAGUUCUCCUCUGGCAUGGAGGGCAAGAGGAGCUGAAUA GCUAAUGGAUGAUAAAUUGCUAGCCUUUCCCUGCCAAAGGAGAGCUGCCCUGCC AUUCAGUG, an RNA fragment encoding a hairpin motif, the fragment arising from O. sativa;

SEQ ID NO: 896 is AGUCCAGUUUCAGGGCUCCUCUCUCUUGGCAGGGAGCAUGUGAAGUCUUUUGUA GCUCACUCAUUUUCAGCCCUCUGCCAAAGGAGAGUUGCCCUAAAACUGGACU, an RNA fragment encoding a hairpin motif, the fragment arising from O. sativa;

SEQ ID NO: 897 is AGCUGCAUUGCUGGGCAAGUUGUCCUUUGGCAGAUGUUGCAGUUCAUCAUCGAU GCCUGGGGGUUACCAGACUACUGCCAAAGGAAAUUUGCCCCOGAAUUCAUCU, an RNA fragment encoding a hairpin motif, the fragment arising from O. sativa;

SEQ ID NO: 898 is UGACAGAAGAGAGUGAGCACA, an miRNA sequence arising from Brassica;

SEQ ID NO: 899 is UUGACAGAAGAUAGAGAGCAC, an miRNA sequence arising from Glycine;

SEQ ID NO: 900 is UUGACAGAAGAUAGAGAGCAC, an miRNA sequence arising from Glycine;

SEQ ID NO: 901 is UGACAGAAGAGAGUGAGCACA, an miRNA sequence arising from Glycine;

SEQ ID NO: 902 is UGACAGAAGAGAGAGAGCACA, an miRNA sequence arising from Glycine;

SEQ ID NO: 903 is UUGACAGAAGAUAGAGAGCAC, an miRNA sequence arising from Helianthus;

SEQ ID NO: 904 is UUGACAGAAGAGAGAGAGCAC, an miRNA sequence arising from Lotus;

SEQ ID NO: 905 is UUGACAGAAGAUAGAGGGCAC, an miRNA sequence arising from Medicago;

SEQ ID NO: 906 is UUGACAGAAGAUAGAGAGCAC, an miRNA sequence arising from Nicotiana;

SEQ ID NO: 907 is UGACAGAAGAGAGUGAGCACA, an miRNA sequence arising from Oryza;

SEQ ID NO: 908 is UGACAGAAGAGAGUGAGCACA, an miRNA sequence arising from Oryza;

SEQ ID NO: 909 is UGACAGAAGAGAGUGAGCACA, an miRNA sequence arising from Saccharum;

SEQ ID NO: 910 is UGACAGAAGAGAGUGAGCACA, an miRNA sequence arising from Saccharum;

SEQ ID NO: 911 is UUGACAGAAGAGAGAGAGCAC, an miRNA sequence arising from Sesamum;

SEQ ID NO: 912 is UUGACAGAAGAUAGAGAGCAC, an miRNA sequence arising from Solanum;

SEQ ID NO: 913 is UGACAGAAGAGAGUGAGCACA, an miRNA sequence arising from Solanum;

SEQ ID NO: 914 is UGACAGAAGAGAGUGAGCACA, an miRNA sequence arising from Sorghum;

SEQ ID NO: 915 is UGACAGAAGAGAGAGAGCAUG, an miRNA sequence arising from Vitis;

SEQ ID NO: 916 is UGACAGAAGAGAGUGGGCACA, an miRNA sequence arising from Zea;

SEQ ID NO: 917 is UGACAGAAGAGAGUGAGCACA, an miRNA sequence arising from Zea;

SEQ ID NO: 918 is UGACAGAAGAGAGUGAGCACA, an miRNA sequence arising from Zea;

SEQ ID NO: 919 is UGACAGAAGAGAGUGAGCACA, an miRNA sequence arising from Zea;

SEQ ID NO: 920 is UGACAGAAGAGAGUGAGCACA, an miRNA sequence arising from Zea;

SEQ ID NO: 921 is UGACAGAAGAGAGUGAGCACA, an miRNA sequence arising from Zea;

SEQ ID NO: 922 is UUGGACUGAAGGGAGCUCCC, an miRNA sequence arising from Glycine;

SEQ ID NO: 923 is UUGGACUGAAAGGAGCUCCU, an miRNA sequence arising from Glycine;

SEQ ID NO: 924 is UUUGGAUUGAAGGGAGCUCUA, an miRNA sequence arising from Glycine;

SEQ ID NO: 925 is AUUGGAGUGAAGGGAGCUCCA, an miRNA sequence arising from Glycine;

SEQ ID NO: 926 is UUGGACUGAAGGGAGCUCCC, an miRNA sequence arising from Glycine;

SEQ ID NO: 927 is UUUGGAUUGAAGGGAGCUCUG, an miRNA sequence arising from Hordeum;

SEQ ID NO: 928 is UUGGACUGAAGGGAGCUCCC, an miRNA sequence arising from Liriodendron;

SEQ ID NO: 929 is UUUGGAUUGAAGGGAGCUCUA, an miRNA sequence arising from Medicago;

SEQ ID NO: 930 is UUUGGAUUGAAGGGAGCUCUG, an miRNA sequence arising from Oryza;

SEQ ID NO: 931 is UUUGGAUUGAAGGGAGCUCUG, an miRNA sequence arising from Oryza;

SEQ ID NO: 932 is UUUGGAUUGAAGGGAGCUCUG, an miRNA sequence arising from Pennisetum;

SEQ ID NO: 933 is UUGGACUGAAGGGAGCUCCA, an miRNA sequence arising from Physcomitrella;

SEQ ID NO: 934 is UUUGGAUUGAAGGGAGCUCUG, an miRNA sequence arising from Saccharum;

SEQ ID NO: 935 is UUUGGAUUGAAGGGAGCUCUG, an miRNA sequence arising from Saccharum;

SEQ ID NO: 936 is UUUGGAUUGAAGGGAGCUCUG, an miRNA sequence arising from Saccharum;

SEQ ID NO: 937 is UUGGAUCGAAGGGAGCUCUU, an miRNA sequence arising from Saccharum;

SEQ ID NO: 938 is CUUGGAUUGAAGGGAGCUCCU, an miRNA sequence arising from Saccharum;

SEQ ID NO: 939 is UUUGGAUUGAAAGGAGCUCUU, an miRNA sequence arising from Saccharum;

SEQ ID NO: 940 is UUUGGAUUGAAGGGAGCUCUG, an miRNA sequence arising from Schedonorus;

SEQ ID NO: 941 is UUUGGAUUGAAGGGAGCUCUG, an miRNA sequence arising from Sorghum;

SEQ ID NO: 942 is UUUGGAUUGAAGGGAGCUCUG, an miRNA sequence arising from Sorghum;

SEQ ID NO: 943 is UUUGGAUUGAAGGGAGCUCUG, an miRNA sequence arising from Sorghum;

SEQ ID NO: 944 is UUGGACUGAAGGGAGCUCCC, an miRNA sequence arising from Triticum;

SEQ ID NO: 945 is UUUGGAUOGAAGGGAGCUCUG, an miRNA sequence arising from Triticum;

SEQ ID NO: 946 is UUUGGAUUGAAGGGAGCUCUG, an miRNA sequence arising from Triticum;

SEQ ID NO: 947 is UUUGGAUUGAAGGGAGCUCUA, an miRNA sequence arising from Vitis;

SEQ ID NO: 948 is UUUGGACUGAAGGGAGCUCCU, an miRNA sequence arising from Vitis;

SEQ ID NO: 949 is UUUGGAUUGAAGGGAGCUCUA, an miRNA sequence arising from Vitis;

SEQ ID NO: 950 is CUUGGAUUGAAGGGAGCUCCU, an miRNA sequence arising from Zea;

SEQ ID NO: 951 is UUGGAUCGAAGGGAGCUCUU, an miRNA sequence arising from Zea;

SEQ ID NO: 952 is UGCCUGGCUCCCUGUAUGCCA, an miRNA sequence arising from Glycine;

SEQ ID NO: 953 is UGCCUGGCUCCCUGUAUGCCA, an miRNA sequence arising from Oryza;

SEQ ID NO: 954 is UGCCUGGCUCCCUGUAUGCCA, an miRNA sequence arising from Oryza;

SEQ ID NO: 955 is UGCCUGGCUCCCUGUAUGCCA, an miRNA sequence arising from Triticum;

SEQ ID NO: 956 is UGCCUGGCUCCCUGUAUGCCA, an miRNA sequence arising from Zea;

SEQ ID NO: 957 is UCGAUAAACCUCUGCAUCCAG, an miRNA sequence arising from Lupinus;

SEQ ID NO: 958 is UCGAUAAACCUCUGCAUCCAG, an miRNA sequence arising from Medicago;

SEQ ID NO: 959 is UCGAUAAGCCUCUGCAUCCAG, an miRNA sequence arising from Oryza;

SEQ ID NO: 960 is UCGAUAAACCUCUGCAUCCAG, an miRNA sequence arising from Vitis;

SEQ ID NO: 961 is UGGAGAAGCAGGGCACGUGCA, an miRNA sequence arising from Populus;

SEQ ID NO: 962 is UGGAGAAGCAGGGCACGUGCA, an miRNA sequence arising from Populus;

SEQ ID NO: 963 is UGGAGAAGCAGGGCACGUGCA, an miRNA sequence arising from Triticum;

SEQ ID NO: 964 is UCGGACCAGGCUUCAUUCCCU, an miRNA sequence arising from Glycine;

SEQ ID NO: 965 is UCGGACCAGGCUUCAUUCCCC, an miRNA sequence arising from Glycine;

SEQ ID NO: 966 is UCGGACCAGGCUUCAUUCCCG, an miRNA sequence arising from Glycine;

SEQ ID NO: 967 is UCGGACCAGGCUUCAUUCCCC, an miRNA sequence arising from Glycine;

SEQ ID NO: 968 is UCGGACCAGGCUUCAUUCCCC, an miRNA sequence arising from Glycine;

SEQ ID NO: 969 is UCGGACCAGGCUUCAUCCCCC, an miRNA sequence arising from Hedyotis;

SEQ ID NO: 970 is UCGGACCAGGCUUCAUUCCCC, an miRNA sequence arising from Hordeum;

SEQ ID NO: 971 is UCGGACCAGGCUUCAUUCCUC, an miRNA sequence arising from Ipomoea;

SEQ ID NO: 972 is UCGGACCAGGCUJCAUUCCCC, an miRNA sequence arising from Medicago;

SEQ ID NO: 973 is UCGGACCAGGCUUCAUUCCCC, an miRNA sequence arising from Medicago;

SEQ ID NO: 974 is UCGGACCAGGCUUCAUUCCCC, an miRNA sequence arising from Medicago;

SEQ ID NO: 975 is UCGGACCAGGCUUCAUUCCUC, an miRNA sequence arising from Medicago;

SEQ ID NO: 976 is UCGGACCAGGCUUCAAUCCCU, an miRNA sequence arising from Oryza;

SEQ ID NO: 977 is UCGGACCAGGCUUCAUUCCCC, an miRNA sequence arising from Sorghum;

SEQ ID NO: 978 is UCGGACCAGGCUUCAUUCCCC, an miRNA sequence arising from Zea;

SEQ ID NO: 979 is UGAAGCUGCCAGCAUGAUCUG, an miRNA sequence arising from Glycine;

SEQ ID NO: 980 is UGAAGCUGCCAGCAUGAUCUA, an miRNA sequence arising from Glycine;

SEQ ID NO: 981 is UGAAGCUGCCAGCAUGAUCUG, an miRNA sequence arising from Oryza;

SEQ ID NO: 982 is UGAAGCUGCCAGCAUGAUCUG, an miRNA sequence arising from Oryza;

SEQ ID NO: 983 is UGAAGCUGCCAGCAUGAUCUU, an miRNA sequence arising from Phaseolus;

SEQ ID NO: 984 is UGAAGCUGCCAGCAUGAUCUG, an miRNA sequence arising from Saccharum;

SEQ ID NO: 985 is UGAAGCUGCCAGCAUGAUCUG, an miRNA sequence arising from Saccharum;

SEQ ID NO: 986 is UGAAGCUGCCAGCAUGAUCUG, an miRNA sequence arising from Saccharum;

SEQ ID NO: 987 is UGAAGCUGCCAGCAUGAUCUG, an miRNA sequence arising from Zea;

SEQ ID NO: 988 is UCGCUUGGUGCAGGUCGGGAA, an miRNA sequence arising from Arabidopsis;

SEQ ID NO: 989 is UCGCUUGGUGCAGGUCGGGAA, an miRNA sequence arising from Betula;

SEQ ID NO: 990 is UCGCUUGGUGCAGGUCGGGAA, an miRNA sequence arising from Glycine;

SEQ ID NO: 991 is UCGCUUGGUGCAGGUCGGGAA, an miRNA sequence arising from Hedyotis;

SEQ ID NO: 992 is UCGCUUGGUGCAGGUCGGGAC, an miRNA sequence arising from Lycopersicon;

SEQ ID NO: 993 is UCGCUUGGUGCAGGUCGGGAC, an miRNA sequence arising from Lycopersicon;

SEQ ID NO: 994 is UCGCUUGGUGCAGAUCGGGAC, an miRNA sequence arising from Oryza;

SEQ ID NO: 995 is UCGCUUGGUGCAGGUCGGGAA, an miRNA sequence arising from Populus;

SEQ ID NO: 996 is UCGCUUGGUGCAGGUCGGGAA, an miRNA sequence arising from Populus;

SEQ ID NO: 997 is UCGCUUGGUGCAGAUCGGGAC, an miRNA sequence arising from Saccharum;

SEQ ID NO: 998 is UCGCUUGGUGCAGAUCGGGAC, an miRNA sequence arising from Saccharum;

SEQ ID NO: 999 is UCGCUUGGUGCAGGUCGGGAC, an miRNA sequence arising from Solanum;

SEQ ID NO: 1000 is UCGCUUGGUGCAGGUCGGGAC, an miRNA sequence arising from Solanum;

SEQ ID NO: 1001 is UCGCUUGGUGCAGGUCGGGAC, an miRNA sequence arising from Solanum;

SEQ ID NO: 1002 is UCGCUUGGUGCAGAUCGGGAC, an miRNA sequence arising from Sorghum;

SEQ ID NO: 1003 is UCGCUUGGUGCAGGUCGGGAA, an miRNA sequence arising from Vitis;

SEQ ID NO: 1004 is UCGCUUGGUGCAGAUCGGGAC, an miRNA sequence arising from Zea;

SEQ ID NO: 1005 is UCGCUUGGUGCAGAUCGGGAC, an miRNA sequence arising from Zea;

SEQ ID NO: 1006 is UCGCUUGGUGCAGAUCGGGAC, an miRNA sequence arising from Zea;

SEQ ID NO: 1007 is CAGCCAAGGAUGACUUGCCGG, an miRNA sequence arising from Glycine;

SEQ ID NO: 1008 is CAGCCAAGGAUGACUUGCCGG, an miRNA sequence arising from Glycine;

SEQ ID NO: 1009 is CAGCCAAGGAUGACUUGCCGG, an miRNA sequence arising from Glycine;

SEQ ID NO: 1010 is CAGCCAAGGAUGACUUGCCGA, an miRNA sequence arising from Oryza;

SEQ ID NO: 1011 is CAGCCAAGGAUGACUUGCCGG, an miRNA sequence arising from Oryza;

SEQ ID NO: 1012 is UAGCCAAGGAGACUGCCUAUG, an miRNA sequence arising from Oryza;

SEQ ID NO: 1013 is CAGCCAAGGAUGACUUGCCGA, an miRNA sequence arising from Oryza;

SEQ ID NO: 1014 is UAGCCAAGGAGACUGCCCAUG, an miRNA sequence arising from Oryza;

SEQ ID NO: 1015 is UAGCCAAGGAGACUGCCUAUC, an miRNA sequence arising from Oryza;

SEQ ID NO: 1016 is UAGCCAAGAAUGGCUUGCCUA, an miRNA sequence arising from Oryza;

SEQ ID NO: 1017 is CAGCCAAGGAUGACUUGCCGA, an miRNA sequence arising from Populus;

SEQ ID NO: 1018 is CAGCCAAGGAUGAUUUGCCGA, an miRNA sequence arising from Populus;

SEQ ID NO: 1019 is UAGCCAAGGAUGAUUUGCCUG, an miRNA sequence arising from Triticum;

SEQ ID NO: 1020 is UAGCCAAGGACAGACUUGCCG, an miRNA sequence arising from Zea;

SEQ ID NO: 1021 is UGAUUGAGCCGCGCCAAUAUC, an miRNA sequence arising from Arabidopsis;

SEQ ID NO: 1022 is UCAUUGAGCCGUGCCAAUAUC, an miRNA sequence arising from Glycine;

SEQ ID NO: 1023 is CGAUUGAGCCGUGCCAAUAUC, an miRNA sequence arising from Hedyotis;

SEQ ID NO: 1024 is UGAUUGAGCCGUGCCAAUAUC, an miRNA sequence arising from Hordeum;

SEQ ID NO: 1025 is UGAUUGAGCCGUGCCAAUAUC, an miRNA sequence arising from Oryza;

SEQ ID NO: 1026 is UGAUUGAGCCGUGCCAAUAUC, an miRNA sequence arising from Oryza;

SEQ ID NO: 1027 is CGAUUGAGCCGUGCCAAUAUC, an miRNA sequence arising from Triticum;

SEQ ID NO: 1028 is UGAUUGAGCCGUGCCAAUAUC, an miRNA sequence arising from Triticum;

SEQ ID NO: 1029 is UGAUUGAGCCGUGCCAAUAUC, an miRNA sequence arising from Zea;

SEQ ID NO: 1030 is UGAUUGAGCCGUGCCAAUAUC, an miRNA sequence arising from Zea;

SEQ ID NO: 1031 is AGAAUCUUGAUGAUGCUGCAA, an miRNA sequence arising from Citrus;

SEQ ID NO: 1032 is AGAAUCUUGAUGAUGCUGCAU, an miRNA sequence arising from Glycine;

SEQ ID NO: 1033 is AGAAUCUUGAUGAUGCUGCAU, an miRNA sequence arising from Glycine;

SEQ ID NO: 1034 is AGAAUCUUGAUGAUGCUGCAU, an miRNA sequence arising from Lycopersicon;

SEQ ID NO: 1035 is AGAAUCUUGAUGAUGCUGCAU, an miRNA sequence arising from Solanum;

SEQ ID NO: 1036 is UCCAAAGGGAUCGCAUUGAUC, an miRNA sequence arising from Oryza;

SEQ ID NO: 1037 is UCCAAAGGGAUCGCAUUGAUC, an miRNA sequence arising from Oryza;

SEQ ID NO: 1038 is UCCAAAGGGAUCGCAUUGAUC, an miRNA sequence arising from Populus;

SEQ ID NO: 1039 is UUGGCAUUCUGUCCACCUCC, an miRNA sequence arising from Glycine;

SEQ ID NO: 1040 is UUGGCAUUCUGUCCACCUCC, an miRNA sequence arising from Glycine;

SEQ ID NO: 1041 is UUGGCAUUCUGUCCACCUCC, an miRNA sequence arising from Robinia;

SEQ ID NO: 1042 is UGAAGUGUUUGGGGGAACUCU, an miRNA sequence arising from Glycine;

SEQ ID NO: 1043 is GUGAAGUGUUUGGGGGAACUC, an miRNA sequence arising from Oryza;

SEQ ID NO: 1044 is GUGAAGUGUUUGGGGGAACUC, an miRNA sequence arising from Oryza;

SEQ ID NO: 1045 is AUGAAGUGUUUGGAGGAACUC, an miRNA sequence arising from Oryza;

SEQ ID NO: 1046 is GUGAAGUGCUUGGGGGAACUC, an miRNA sequence arising from Oryza;

SEQ ID NO: 1047 is GUGAAGUGUUUUGGGGGAACUC, an miRNA sequence arising from Oryza;

SEQ ID NO: 1048 is GUGAAGUGUUUGGAGGAACUC, an miRNA sequence arising from Oryza;

SEQ ID NO: 1049 is GUGAAGUGUUUGGGGGAACUC, an miRNA sequence arising from Oryza;

SEQ ID NO: 1050 is GUGAAGUGUUUGGAGGGACUC, an miRNA sequence arising from Triticum;

SEQ ID NO: 1051 is AUGAAGUGUUUGGGGGAACUC, an miRNA sequence arising from Triticum;

SEQ ID NO: 1052 is UUCCACAGCUUUCUUGAACUU, an miRNA sequence arising from Brassica;

SEQ ID NO: 1053 is UUCCACAGCUUUCUUGAACUU, an miRNA sequence arising from Glycine;

SEQ ID NO: 1054 is UUCCACAGCUUUCUUGAACUG, an miRNA sequence arising from Glycine;

SEQ ID NO: 1055 is UUCCACAGCUUUCUUGAACUG, an miRNA sequence arising from Mesembryanthemum;

SEQ ID NO: 1056 is UUCCACAGCUUUCUUGAACUG, an miRNA sequence arising from Mesembryanthemum;

SEQ ID NO: 1057 is UUCCUCAGCUUUCUUGAACUG, an miRNA sequence arising from Mesembryanthemum;

SEQ ID NO: 1058 is UCCCACAGCUUUCUUGAACUU, an miRNA sequence arising from Mesembryanthemum;

SEQ ID NO: 1059 is UUCCACAGCUUUCUOGAACUG, an miRNA sequence arising from Oryza;

SEQ ID NO: 1060 is UUCCACAGCUUUCUUGAACUG, an miRNA sequence arising from Populus;

SEQ ID NO: 1061 is UUCCACAGCUUUCUUGAACUG, an miRNA sequence arising from Populus;

SEQ ID NO: 1062 is UUCCACAGCUUUCUUGAACUG, an miRNA sequence arising from Prunus;

SEQ ID NO: 1063 is UUCCACAGCUUUCUUGAACUG, an miRNA sequence arising from Prunus;

SEQ ID NO: 1064 is UUCCACAGCUUUCUUGAACUG, an miRNA sequence arising from Saccharum;

SEQ ID NO: 1065 is UUCCACAGCUUUCUUGAACUU, an miRNA sequence arising from Solanum;

SEQ ID NO: 1066 is UUCCACAGCUUUCUUGAACUG, an miRNA sequence arising from Zea;

SEQ ID NO: 1067 is UUCCACAGCUUUCUUGAACUG, an miRNA sequence arising from Zea;

SEQ ID NO: 1068 is CGUUGAGUGCAGCGUUGAUG, an miRNA sequence arising from Hordeum;

SEQ ID NO: 1069 is CGUTJGAGUGCAGCGUUGAUG, an miRNA sequence arising from Hordeum;

SEQ ID NO: 1070 is UGUGLTUCUCAGGUCACCCCUU, an miRNA sequence arising from Citrus;

SEQ ID NO: 1071 is UGUGUUCUCAGGUCGCCCCUG, an miRNA sequence arising from Glycine;

SEQ ID NO: 1072 is UGUGUUCUCAGGUCACCCCUU, an miRNA sequence arising from Glycine;

SEQ ID NO: 1073 is UGUGUUCUCAGGUCACCCCUU, an miRNA sequence arising from Glycine;

SEQ ID NO: 1074 is CGUGUUCUCAGGUCGCCCCUG, an miRNA sequence arising from Helianthus;

SEQ ID NO: 1075 is UGUGUUCUCAGGUCGCCCCUG, an miRNA sequence arising from Lactuca;

SEQ ID NO: 1076 is UGUGUUCUCAGGUCGCCCCUG, an miRNA sequence arising from Lactuca;

SEQ ID NO: 1077 is UGUGUUCUCAGGUCACCCCUU, an miRNA sequence arising from Lotus;

SEQ ID NO: 1078 is UGUGUUCUCAGGUCACCCCUU, an miRNA sequence arising from Medicago;

SEQ ID NO: 1079 is UGUGUUCUCAGGUCGCCCCUG, an miRNA sequence arising from Medicago;

SEQ ID NO: 1080 is UGUGUUCUCAGGUCGCCCCUG, an miRNA sequence arising from Nicotiana;

SEQ ID NO: 1081 is UGUGUUCUCAGGUCGCCCCUG, an miRNA sequence arising from Oryza;

SEQ ID NO: 1082 is UGUGUUCUCAGGUCGCCCCCG, an miRNA sequence arising from Zea;

SEQ ID NO: 1083 is UGCCAAAGGAGAGUUGCCCUG, an miRNA sequence arising from Medicago;

SEQ ID NO: 1084 is UGCCAAGGGAGAAUUGCCCUG, an miRNA sequence arising from Populus;

SEQ ID NO: 1085 is CAUAGCAACUGACAGAAGAGAGUGAGCACACAAAAGUAAUCUGCAUAUACUGCA UUUGCUUCUCUUGCGUGCUCACUGCUCUUUCUGUCAGAUUCUAGU, an RNA fragment encoding a hairpin motif, the fragment arising from Brassica;

SEQ ID NO: 1086 is UUAAGGUUGUUGACAGAAGAUAGAGAGCACAGAUGAUGAUAUGCAUAUUAUAU AAUAUAUAGCAGGGAACUCAUGAUGAAUUGUGCAUCUUACUCCUUUGUGCUCUC UAUACUUCUGUCAUCACCUUCAG, an RNA fragment encoding a hairpin motif, the fragment arising from Glycine;

SEQ ID NO: 1087 is GUGAUGCUGUUGACAGAAGAUAGAGAGCACUGAUGAUGAAAUGCAUGAAAGGG. AAUGGCAUCUCACUCCUUUGUGCUCUCUAGUCUUCUGUCAUCAUCCUUCU, an RNA fragment encoding a hairpin motif, the fragment arising from Glycine;

SEQ ID NO: 1088 is GAGAGAGGCUGACAGAAGAGAGUGAGCACAUGCUAGUGGUAUUUGUAUGAGGG CAUACAAUUGCGGGUGCGUGCUCACUUCUCUAUCUGUCAGCUUCCCAU, an RNA fragment encoding a hairpin motif, the fragment arising from Glycine;

SEQ ID NO: 1089 is AUCUCAUGUUGACAGAAGAGAGAGAGCACAACCCGGGAAUGGCUAAAGGAGUC UUUGCCUUUGUUGGGAGUGUGCCCUCUCUUCCUCUGUCAUCAUCACAU, an RNA fragment encoding a hairpin motif, the fragment arising from Glycine;

SEQ ID NO: 1090 is UGAUGGAUGUUGACAGAAGAUAGAGAGCACAGAGAAGCAUGAAUUGCACAUAG AUAUUGCAAUUCACUCCUUCGUGCUCUCUAUGCUUCUGUCAUUACCUAUUA, an RNA fragment encoding a hairpin motif, the fragment arising from Helianthus;

SEQIDNO: 1091 is UUCAUGCAUGUUGACAGAAGAGAGAGAGCACAACCCAGGAAUGGUGAAAGAGA GUCUUUGCUUUUGUUGGGAGUGUGCUCUCCCUUCUUCUGUCAUCAUCACAUGA, an RNA fragment encoding a hairpin motif, the fragment arising from Lotus;

SEQ ID NO: 1092 is GUAAGGUUGUUGACAGAAGAUAGAGGGCACUAAGGAUGAUAUGCAUACACAUA UAUAUACAACAUGGAGGAGGAGCUUAAUUGCAUUUCAUUUCCUUUGUGCUCUC UAGACUUCUGUCAUCACCUCAUC, an RNA fragment encoding a hairpin motif, the fragment arising from Medicago;

SEQ ID NO: 1093 is UGUGAGAUUGUUGACAGAAGAUAGAGAGCACAGAUGAUGAUCAUGUCUGCUAA AUCUGGGAUUGGAGAGGGCACUGAAUCAAUUAAACUGCAGAGAAUAAAAAGCA UCUCAAUUCAUUUGUGCUCUCUAUGCUUCCGUCAUCACCUUCACC, an RNA fragment encoding a hairpin motif, the fragment arising from Nicotiana;

SEQ ID NO: 1094 is UGGGAGNUCUGACAGAAGAGAGUGAGCACACACGGUGCUUUCUUAGCAUGCAA GAGCCNAUGCUGGGAGCUGUGCGUGCUCACUCUCUAUCUGUCAGCCCGUUCA, an RNA fragment encoding a hairpin motif, the fragment arising from Oryza;

SEQ ID NO: 1095 is GCGAGAUUGUUGACAGAAGAGAGUGAGCACACGGCGCGGCGGCUAGCCAUCGGC GGGAUGCCUGCCCCCGCCGCGUGCUCGCUCCUCUUUCUGUCAGCAUCUCUCA, an RNA fragment encoding a hairpin motif, the fragment arising from Oryza;

SEQ ID NO: 1096 is GGUGGAGGCUGACAGAAGAGAGUGAGCACACAUGGUGCCUUUCUUGCAUGAUG AACGAUCGAGAGGUUCAUGCUCGAAGCUAUGCGUGCUCACUUCUCUCUCUGUCA GCCGUUAGA, an RNA fragment encoding a hairpin motif, the fragment arising from Saccharum;

SEQ ID NO: 1097 is UUUGAAGGUUUGACAGAAGAGAGUGAGCACACACGAUGGUUUCUUANCAUGAG UGCCAUGCUGGGAGCUGUGCGUGCUCACUCUCUAUCUGUCAGCCACUCAUC, an RNA fragment encoding a hairpin motif, the fragment arising from Saccharum;

SEQ ID NO: 1098 is AUUAAUUUGUUGACAGAAGAGAGAGAGCACAGCCCGCCAUUGACAAAGAGGUC UUUGCCUUUUGUGGGAUUGUGCUCUCUUGCUUCUGCCAACGACCGUCU, an RNA fragment encoding a hairpin motif, the fragment arising from Sesamum;

SEQ ID NO: 1099 is UGAUAAUUGUUGACAGAAGAUAGAGAGCACUAAUGAUGAUAUGCUAAUUUCAU UCAGCAAAAGCAUCUCACUUCAUUUGUGCUCUCUAUGCUUCUGUCAUCACCUUC GC, an RNA fragment encoding a hairpin motif, the fragment arising from Solanum;

SEQ ID NO: 1100 is AAUCAAGACUGACAGAAGAGAGUGAGCACACGCAGUCGAAUUGUAUAAACAUU UAUACAAUUGUCAUUUGCGUGUGCUCACUUCUCAUUCUGUCAGCUCUCUCA, an RNA fragment encoding a hairpin motif, the fragment arising from Solanum;

SEQ ID NO: 1101 is CUUGAGAGAUUGACAGAAGAGAGUGAGCACACGGCGCGACGAACGGCAUAAUA UGUAUGUCGUCCUCGCCGCGUGCUCACUUCUCUUUCGGUCAGCCUCUUCUG, an RNA fragment encoding a hairpin motif, the fragment arising from Sorghum;

SEQ ID NO: 1102 is UGCCUCACAAUGACAGAAGAGAGAGAGCAUGCUGGUGGGAAAACAAUUACAACC UUUGCUCAUCUGAUCUGGAAAUGCUUGUAAGCGGCAUUCUCUUGGAUUGUAAU CUGAAUUCUGCCUCUAUCAUCAACCUGCCCACAAACGAGUUCCUUCAGCUGAGU GCCUUUCCGGCUUGAGCCUUCUGCAUGAUCAGCUGAGUUCUUUCUGCGCCUUUC AUUGUGUCCUG, an RNA fragment encoding a hairpin motif, the fragment arising from Vitis;

SEQ ID NO:1103is AGGUGAAAGCUGACAGAAGAGAGUGGGCACACAUGGUGCCUUUCUUGCAUGAU GUAUGAUCGAGAGAGUUCAUGCUCGAAGCUAUGCGUGCUCACUUCUCUCUCUGU CAGCCAUUAGAA, an RNA fragment encoding a hairpin motif, the fragment arising from Zea;

SEQ ID NO: 1104 is UUGAAGGUUUGACAGAAGAGAGUGAGCACACACGGUGGUUUCUUACCAUGAGU GUCAUGCUAGGAGCUGUGCGUGCUCACUCUCUAUCUGUCAGCCACUCAU, an RNA fragment encoding a hairpin motif, the fragment arising from Zea;

SEQ ID NO:1105is AGGUGAAAGCUGACAGAAGAGAGUGAGCACACAUGGUGCCUUUCUUGCAUGAU GUAUGAUCGAGAGAGUUCAUGCUCGAAGCUAUGCGUGCUCACUUCUCUCUCUGU CAGCCAUUAGAA, an RNA fragment encoding a hairpin motif, the fragment arising from Zea;

SEQ ID NO: 1106 is UUGAAGGUUUGACAGAAGAGAGUGAGCACACACGGUGGUUUCUUACCAUGAGU GUCAUGCUAGGAGCUGUGCGUGCUCACUCUCUAUCUGUCAACCACUCAU, an RNA fragment encoding a hairpin motif, the fragment arising from Zea;

SEQ ID NO: 1107 is UUUGAAGGUUUGACAGAAGAGAGUGAGCACACACGGUGGUUCCUUACCAUGAG UGUCAUGCUAGGAGCUGUGCGUGCUCACUCUCUAUCUGUCAGCCACUCAUC, an RNA fragment encoding a hairpin motif, the fragment arising from Zea;

SEQ ID NO: 1108 is UCGAGAGAUUGACAGAAGAGAGUGAGCACACGGCGCGACGAACGACAUAGCAUG UAUGCCGUCCUCGCCGCGUGCUCACUUCUCUUUCUGUCAGCCUCUUUC, an RNA fragment encoding a hairpin motif, the fragment arising from Zea;

SEQ ID NO: 1109 is GCGACGGUAAGAGAGCUUUCUUCAGUCCACUUAUGGGUGACAAUAAGAUUUCA AUUAGCUGCCGACUCAUUCAUCCAAAUGCUGAGUGAAAGCGAAGAAAGAUACUC AGCAAAUGAGUGAAUGAUGCGGGAGACAAAUUGAUUCUUAAGUGUCCUGUACU UGGACUGAAGGGAGCUCCCUUUUUCUUUU, an RNA fragment encoding a hairpin motif, the fragment arising from Glycine;

SEQ ID NO: 1110 is AAGAGAGUGAAGGAGCUUCCCUCAGCCCAUUCAUGGAGAUAACGAAAGAUUGG GUUGCUGAAUUAACUGCUAGCUCACACAUUCAUUCAUACAAUAGUAUUCAAUUA GGGUAAUAUUGUGUGAAUGAAGCGGGAGUAUAUAGUAUCUAUAUUGCAACCCU CUUUCUCUGUGCUUGGACUGAAAGGAGCUCCUUCUUUUUCUG, an RNA fragment encoding a hairpin motif, the fragment arising from Glycine;

SEQ ID NO: 1111 is AUUAUGAAGUGGAGCUCCUUGAAGUCCAAUUGAGGAUCUUACUGGGUGAAUUG AGCUGCUUAGCUAUGGAUCCCACAGUUCUACCCAUCAAUAAGUGCUUUUGUGGU AGUCUUGUGGCUUCCAUAUCUGGGGAGCUUCAUUUGCCUUUAUAGUAUUAACCU UCUUUGGAUUGAAGGGAGCUCUACACCCUUCU, an RNA fragment encoding a hairpin motif, the fragment arising from Glycine;

SEQ ID NO: 1112 is AAACCCAACUUGGAGUUCCCUGCACUCCAAGUCUGAAAGGAUAUGAUGGUAAAC CUCUACUGCUAGUUCAUGGAUACCUCUGACUUCUUAACAACAUGCGUUCGAAGU CAAGGGUUUGCAUGCCCUGGGAGAUGAGUUUACCUUGAUCUUUUGGUAUUGGA GUGAAGGGAGCUCCAGAGGGUAUUC, an RNA fragment encoding a hairpin motif, the fragment arising from Glycine;

SEQ ID NO: 1113 is CCUAAGGUAAGAGAGCUUUCUUCAGUCCACUCAUGGGUGACAGUAAGAUUCAAU UAGCUGCCGACUCAUUCAUCCAAAUGUUGAGUGUAAGCGAAUAAAUAUACUCAG CAGAUGAGUGAAUGAUGCGGGAGACAAAUUGAAUCUUAAGUUUCCUGUACUUG GACUGAAGGGAGCUCCCUUUUCCUUUU, an RNA fragment encoding a hairpin motif, the fragment arising from Glycine;

SEQ ID NO: 1114 is GUUUGGAGGUGGAGCUCCUAUCAUUCCAAUGAAGGGUCUACCGGAAGGGUUUG UGCAGCUGCUUGUUCAUGGUUCCCACUAUCCUAUCUCCAUUAGAACACGAGGAG AUAGGCUUGUGGUUUGCAUGAUCGAGGAGCCGCUUCGAUCCCUCGCUGACCGCU GUUUGGAUUGAAGGGAGCUCUGCAUCUUGAU, an RNA fragment encoding a hairpin motif, the fragment arising from Hordeum;

SEQ ID NO: 1115 is GUUAUGGACUAAGGAGCUCUCUUCAGUCCAGUCCAAGAUAGUAUUAAGCCAAUC UCCGCUGCUGACUCGUUGGCUCAUGAACUCAUCCAACGGCUAGGAUUCCGAUGU GUUUUUGAUCCAACGAUGCGGGAGCCGUGUUUGGUUCUGUCUGUCUCGUCUUGG ACUGAAGGGAGCUCCCUUCUGUUCCAC, an RNA fragment encoding a hairpin motif, the fragment arising from Liriodendron;

SEQ ID NO: 1116 is UUAAAGGGGUGGAGCUUCCUUUAGUCCAAAUAUGGAUCUUGCUAUGUUGAUAG AGCUGCUUAGCUAUGGGUCCCUCAACUCUACCCAUCUUGUUCUUUGUGGUAGUU UUGUGGCUUCCAUAUCUAGGGAGCCUUAUCACCUUUAGUUUAAUCUUUCUUUGG AUUGAAGGGAGCUCUACAUCUUGCU, an RNA fragment encoding a hairpin motif, the fragment arising from Medicago;

SEQ ID NO: 1117 is UUGUGGACGUUGAGCUCCUUUCGGUCCAAAAAGGGGUGUUGCUGUGGGUCGAU UGAGCUGCUGGGUCAUGGAUCCCGUUAGCCUACUCCAUGUUCAUCAUUCAGCUC GAGAUCUGAAAGAAACUACUCCAAUUUAUACUAAUAGUAUGUGUGUAGAUAGG AAAAUGAUGGAGUACUCGUUGUUGGGAUAGGCUUAUGGCUUGCAUGCCCCAGG AGCUGCAUCAACCCUACAUGGACCCUCUUUGGAUUGAAGGGAGCUCUGCAUCUU UUG, an RNA fragment encoding a hairpin motif, the fragment arising from Oryza;

SEQ ID NO: 1118 is UUGUGGACGUUGAGCUCCUUUCGGUCCAAAAAGGGGUGUUGCUGUGGGUCGAU UGAGCUGCUGGGUCAUGGAUCCCGUUAGACUACUCCAUGUUCAUCAUUCAGCUC GAGAUCUGAAAGAAACUACUCCAAUUUAUACUAAUAGUAUGUGUGUAGAUAGG AAAAUGAUGGAGUACUCGUUGGGAUAGGCUUAUGGCUUGCAUGCCCCAGGAGC UGCAUCAACCCUACAUGGACCCUCUUUGGAUUGAAGGGAGCUCUGCAUCUUUGG an RNA fragment encoding a hairpin motif, the fragment arising from Oryza;

SEQ ID NO: 1119 is GAUUGGAAGCGGAGCUCCUAUCAUUCCAAUGAAAGGUUGUUUGUGGGUUGG UACAGCUGCUCGUUCAUGGUUCCCACUAUCCUAUCUCAUGGCUGGAGGUUUAUC UGAGAGAGAGAGAGAGAGAUGAGAUGAGUGGUCGGUCUGGUGUUGGCUUGAGA UAGGCUUGUGGCUUGCAUGACCGAGGAGCUGCACCGUCCCCUUGCUGGCCGCUC UUUGGAUUGAAGGGAGCUCUGCAUCUUGAU, an RNA fragment encoding a hairpin motif, the fragment arising from Pennisetum;

SEQ ID NO: 1120 is ACCUUGAUUGUGGAGCUCCGUUUUCGGUCCAAUAGUGGCUGCGACGGAAGGUGG UCCCGCUGCCGAAUCACACGUCCGGGUUCUUUAUCGGGGGCAGGGCCCCGAUAC GGUAUCCGAACGUUUGUCCCGGGAACUGGUCGACCUUCCGCCCGGCGUCUCUUG GACUGAAGGGAGCUCCACUCUUGGCGU, an RNA fragment encoding a hairpin motif, the fragment arising from Physcomitrella;

SEQ ID NO: 1121 is UUUGAAGCGGAGCUCCUAUCAUUCCAAUGAAGGGCCGUUCUGAAGGGUUGUUCC GCUGCUCGUUCAUGGUUCCCACUAUCCUAUCUCAUCAUGUAUGUGUGUAUGUAU UUUCGAGAGGGAGGAGAGGAGCUAGACUCUCAUGGUGGUCGUCUUUGAGAUAG GCUUGUGGUUUGCAUGACCGAGGAGCUGCACCGUCCCCUUGCUGGCCGCUCUUU GGAUUGAAGGGAGCUCUGCAUCCUGAUC, an RNA fragment encoding a hairpin motif, the fragment arising from Saccharum;

SEQ ID NO:1122is GAUUUGAAGCGGAGCUCCUAUCAUUCCAAUGAAGGGCCGUUCUGAAGGGUUGU UCCGCUGCUCGUUCAUGGUUCCCACUAUCCUAUCUCAUCAUGUAUGUGUGUAUG UAUUUUCGAGAGGGAGGAGAGGAGCUAGACUCUCAUGGGGGUCGUCUAUGAGA UACGCUUGUGGUUUGCAUGACCGAUGAGCUGCACCGUCCCCUUGCUGGCCGCUC UUUGGAUUGAAGGGAGCUCUGCAUCCUGAU, an RNA fragment encoding a hairpin motif, the fragment arising from.Saccharum;

SEQ ID NO: 1123 is GAUUUGAAGCGGAGCUCCUAUCAUUCCAAUGAAGGGCCGUUCUGAAGGGUGGU UCCGCUGCUCGUUCAUGGUUCCCACUAUCCUAUCUCAUCAUGUAUGUGUGUAUG UAUUUUCGGAGAGGGAGGAGAGGAGCUAGACUCUCACGGUGGUCGUCUUUGAG AUAGGCUUGUGGUUUGCAUGACCGAGGAGCUGCACCGUCCCCUUGCUGGCCGCU CUUUGGAUUGAAGGGAGCUCUGCAUCCUGAA, an RNA fragment encoding a hairpin motif, the fragment arising from Saccharum;

SEQ ID NO: 1124 is GGAAAGAGAGAGGAGCUCCCUUCAAUCCAAGCACGAGGGAAAGAUGAUGGUGG GUUCAUCUCCCGGGUCAUGCACACCCAUGCAAGUGCAGGUGAGCAUUAGUCAUU GCUGCACCAGAGAGGCAUCCAUGAACCGGCAGCUGCAACCGACCACUUCCCCUC CUGGAUUGGAUCGAAGGGAGCUCUUCGAUCACUUU, an RNA fragment encoding a hairpin motif, the fragment arising from Saccharum;

SEQ ID NO: 1125 is AAGUGAUCGAAGAGCUCCCUUCGAUCCAAUCCAGGAGGGGAAGUGGUCGGUUGC AGCUGCCGGUUCAUGGAUGCCUCUCUGGUGCAGCAAUGACUAAUGCUCACCUGC ACUUGCAUGGGUGUGCAUGACCCGGGAGAUGAACCCACCAUCAUCUUUCCCUCG UGCUUGGAUUGAAGGGAGCUCCUCUCUCUUUC, an RNA fragment encoding a hairpin motif, the fragment arising from Saccharum;

SEQ ID NO: 1126 is GAUUUGAAGCGGAGCUCCUAUCAUUCCAAUGAAGGGCCGUUCUGAAGGGUGGU UCCGCUGCUCGUUCAUGGUUCCCACUAUCCUAUCUCAUCAUGUAUGUGUGUAUG UAUUUCGAGAGGGACGAGAGGAGCUAGACUCUCACGGUGGUCGUCUUUGAGA UAGGCUUGUGGUUUGCAUGACCGAAGAGCUGCACCGUCCCCUUGCUGGCCGCUC UUUGGAUUGAAAGGAGCUCUUGCAUCUGAU, an RNA fragment encoding a hairpin motif, the fragment arising from Saccharum;

SEQ ID NO: 1127 is GUUUUGAGGUGGAGCUCCUAUCAUUCCAAUGAAAGGUCUUGCUAGAAGGGGUG GUACAGCUGCUCGUUCAUGGUUCCCACUAUCCUACCUCCGUUUGAAACCAGGGA GAUAGGCCUGUGGCUUGCAUGACCGAGGAGCCGCAUCGUCCCCUCGCUGGCCGC UCUUUGGAUUGAAGGGAGCUCUGCAUCUAGGC, an RNA fragment encoding a hairpin motif, the fragment arising from Schedonorus;

SEQ ID NO: 1128 is GAUUCGAAGCGGAGCUCCUAUCAUUCCAAUGAAGGGCCCUUUUCAUGGGUGGUU CCGCUGCUCGUUCAUGGUUCCCACUAUCCUAUCUCAUCAUGUAUCUGUGUAUGU ACUCUAGAGGGCCGGAGAAGAGAUUCAUGUGGUCGUCAGUCUUUGAGAUAGGC UUGUGGUUUGCAUGACCGAGGAGCUGCACCGUCCCCUUGCUGGCCGCUCUUUGG AUUGAAGGGAGCUCUGCAUCCUGAU, an RNA fragment encoding a hairpin motif, the fragment arising from Sorghum;

SEQ ID NO: 1129 is GAUUCGAAGCGGAGCUCCUAUCAUUCCAAUGAAGGGCCCUUUUCAUGGGUGGUU CCGCUGCUCGUUCAUGGUUCCCACUAUCCUAUCUCAUCAUGUAUCUGUGUAUGU ACUCUAGAGGGCCGGAGAAGAGAUUCAUGUGGUCGUCAGUCUUUGAGAUAGGC UUGUGAUUUGCAUGACCGAGGAGCUGCACCGUCCCCUUGCUGGCCGCUCUUUGG AUUGAAGGGAGCUCUGCAUCCUGAU, an RNA fragment encoding a hairpin motif, the fragment arising from Sorghum;

SEQ ID NO: 1130 is GAUUCGAAGCGGAGCUCCUAUCAUUCCAAUGAAGGGCCCUUUUCAUGGGUGGUU CCGCUGCUCGUUCAUGGUUCCCACUAUCCUAUCUCAUCAUGUAUGUGUGUAUGU ACUCUAGAGGGCCCGAGAAGAGAUUCAUGUGGUCGUCAGUCUUUGAGAUAGGC UUGUGGUUUGCAUGACCGAGGAGCUGCACCGUCCCCUUGCUGGCCGCUCUUUGG AUUGAAGGGAGCUCUGCAUCCUGAU, an RNA fragment encoding a hairpin motif, the fragment arising from Sorghum;

SEQ ID NO: 1131 is AGUUUGAGGGAGCUCACUUCAGUCCACUCAUGGGAGGUAGCGGGGAUUGAACG AGCUGCCGACUCAUUCACUCGAGCACACAGUAGAUAUGAGACUAGUCCAGGGCA UACCAGUAUGUUACAAUAUGUACUGUGCGAAUGAGCGAAUGCAGCGGGAGAUU GUUCUCUCUUUCCUCCUCCAUGCUUGGACUGAAGGGAGCUCCCUCAUCUCUCA, an RNA fragment encoding a hairpin motif, the fragment arising from Triticum;

SEQ ID NO: 1132 is UUGUGGACGUUGAGCUCCUUUCGGUCCAAAAAGGGGUGUUGCUGUGGGUCGAU UGAGCUGCUGGGUCAUGGAUCCCGUUAGCCUACUCCAUGUUCAUCAUUCAGCUC GAGAUCUGAAAGAAACUACUCCAAUUUAUACUAAUAGUAUGUGUGUAGAUAGG AAAAUGAUGGAGUACUCGUUGUUGGGAUAGGCUUAUGGCUUGCAUGCCCCAGG AGCUGCAUCAACCCUACAUGGACCCUCUUUGGAUUGAAGGGAGCUCUGCAUCUU UGG, an RNA fragment encoding a hairpin motif, the fragment arising from Triticum;

SEQ ID NO:1133is GUUUAGAGGUGGAGCUCCUAUCAUUCCAAUGAAGGGUCUACCGGAAGGGUUUG UGCAGCUGCUCGUUCAUGGUUCCCACUAUCCUAUCUCCAUAGAAAACGAGGAGA GAGGCCUGUGGUUUGCAUGACCGAGGAGCCGCUUCGAUCCCUCGCUGACCGCUG UUUGGAUUGAAGGGAGCUCUGCAUCUUGAU, an RNA fragment encoding a hairpin motif, the fragment arising from Triticum;

SEQ ID NO: 1134 is GUUUUGGAGUGGAGCUCCUUGAAGUCCAAUAGAGGGUCUUACUGGGUAGAUUG AGCUGCUGACUUAUGGAUCCCACAGCCCUAUCCCGUCAAUGGGGGGCAUUGGAU AGGCUUGUGGCUUGCAUAUCUCAGGAGCUGCAUUAUCCAAGCUUAGAUCCUUGU UUGGAUUGAAGGGAGCUCUACACCUCUCU, an RNA fragment encoding a hairpin motif, the fragment arising from Vitis;

SEQ ID NO:1135is CUGCAGAAAUGGGGGUUCCUUUGCAGCCCAAAACAACUCCAUCGCUGAAGAAGA UGAUGAACUUCAUGCUCCUUGUUUUGGACUGAAGGGAGCUCCUAGUUCUUCUC, an RNA fragment encoding a hairpin motif, the fragment arising from Vitis;

SEQ ID NO:1136is GGUUUGGAGUGAGCUCCUUGAGUCCAAUAGAGGUCUUACUGGGUAGAUGAGCU GCUGACUUAUGGAUCCCACAGCCCUAUCCCGUCAAUGGGGGGCAUUGGAUAGGC UUGUGGCUUGCAUAUCUCAGGAGCUGCAUUAUCCAAGUUUAGAUCCUUGUUUG GAUUGAAGGGAGCUCUACACCUCUCU, an RNA fragment encoding a hairpin motif, the fragment arising from Vitis;

SEQ ID NO:1137is AGGCGAUCGAAGAGCUCCCUUCGAUCCAAUCCAGGAGGGGAAGUGGUCGGUUGC AGCUGCCGGUUCAUGGAUACCUCUCUGGUGCAGCAAUGGCCGCUGCUCACCUCU GCACUUGCAUGGGUGUGCAUGACCCGGGAGAUGAGCCCGCCAUCAUCUUUCCCU CGUGCUUGGAUUGAAGGGAGCUCCUCUCUGUCUG, an RNA fragment encoding a hairpin motif, the fragment arising from Zea;

SEQ ID NO:1138is ACAGACAGAGAGGAGCUCCCUUCAAUCCAAGCACGAGGGAAAGAUGAUGGCGGG CUCAUCUCCCGGGUCAUGCACACCCAUGCAAGUGCAGAGGUGAGCAGCGGCCAU UGCUGCACCAGAGAGGUAUCCAUGAACCGGCAGCUGCAACCGACCACUUCCCCU CCUGGAUUGGAUCGAAGGGAGCUCUUCGAUCGCCUU, an RNA fragment encoding a hairpin motif, the fragment arising from Zea;

SEQ ID NO:.1139is AUGUGUAUGUGCCUGGCUCCCUGUAUGCCAUUUGUAGAGCUCAUCGAAGCAUCA AUGACCUUUGUGGAUGGCGUAUGAGGAGCCAAGCAUAUUUCAUA, an RNA fragment encoding a hairpin motif, the fragment arising from Glycine;

SEQ ID NO:1140is AAAGGGGAUAUGCCUGGCUCCCUGUAUGCCACUCGCGUAGCUGCCAAACUCAGU UGAAACAACUGCCUUCUCCCGGCGAGAUUCAGGCAUUGUGUUCGUACGUUUGGC UCUACUGCGGAUGGCGUGCGAGGAGCCAAGCAUGACCGUCUC, an RNA fragment encoding a hairpin motif, the fragment arising from Oryza;

SEQ ID NO: 1141 is CUUGAGAGCGUGCCUGGCUCCCUGUAUGCCACUCAUGUAGCCCAAUCCAUGGUG UGUUUGGAUGCUGUGGGUGGCGUGCAAGGAGCCAAGCAUGCGUGCCAU, an RNA fragment encoding a hairpin motif, the fragment arising from Oryza;

SEQ ID NO: 1142 is UGAUAUGAUGUGCCUGGCUCCCUGUAUGCCACUCAUCCAGAGCAACACCUUUUG CAAUAAGGUUGCCUGCGAUGGAUGGCGUGCACGGAUCCAAGCAUAUCGAACCC, an RNA fragment encoding a hairpin motif, the fragment arising from Triticum;

SEQ ID NO: 1143 is GUGUCGUGUGUGCCUGGCUCCCUGUAUGCCACACAUGUAGCCAACCCGUGGCGU GAUUGGAUGCUGUGGGUGGCGUGCAAGGAGCCAAGCAUGCAUAACAG, an RNA fragment encoding a hairpin motif, the fragment arising from Zea;

SEQ ID NO: 1144 is GUGAAGUCACUGGAAGCAGCGGUUUAUCGAUCUCUUCCUGAAUUUGAUUAACAC AAACCAUGAAUCGAUCGAUAAACCUCUGCAUCCAGCGCUCACUU, an RNA fragment encoding a hairpin motif, the fragment arising from Lupinus;

SEQ ID NO: 1145 is AAGUUCGUCACUGGAUGCAGCGGUUCAUCGAUCUGUUCCUGAAUUUUGUUUGUC UCGUAAAACAAACAUGAAUCGGUCGAUAAACCUCUGCAUCCAGCGCUCACUUU, an RNA fragment encoding a hairpin motif, the fragment arising from Medicago;

SEQ ID NO: 1146 is GGGUGAUGCCUGGGCGCAGUGGUUUAUCGAUCUCUUCCCUGCCUUGUGCUGCUC CGAUCGAUGCCCGUGCUGAUUCUUGAUAAUAUACAACGCAGGAAUCGAUCGAUA AGCCUCUGCAUCCAGAUCUCACUU, an RNA fragment encoding a hairpin motif, the fragment arising from Oryza;

SEQ ID NO: 1147 is UUGAAGUCACUGGAUGCAGCGGUUCAUCGAUCUCUUCCUGAAAUUGUUGUGAA AAAAGCAGAUCAAGAAUCGGUCGAUAAACCUCUGCAUCCAGCGUUCACUC, an RNA fragment encoding a hairpin motif, the fragment arising from Vitis;

SEQ ID NO: 1148 is UGAGCAAGAUGGAGAAGCAGGGCACGUGCAUUACUAACUCAUGCACACAGAGUG AGAGAGACAUUUCUUGCUGGAGUUAUGACUCUUACCUACAAUAGAUUUUGUUG GCUUCAGCGAGUUAGUUCUUCAUGUGCCUGUCUUCCCCAUCAUGAUCA, an RNA fragment encoding a hairpin motif, the fragment arising from Populus;

SEQ ID NO: 1149 is UGAGCAAGAUGGAGAAGCAGGGCACGUGCAUUACUAACUCAUGCAUACAGAGU GAGAGAGACAUUUCUUGCUCGAGUUAUGACUCUUACCUACUAUAGAUUUUGUU GGCCUCAGUGAGUUAGUUCUUCAUGUGCCUGUCUUCCCCAUCAUGAUCA, an RNA fragment encoding a hairpin motif, the fragment arising from Populus;

SEQ ID NO: 1150 is CGCGCGAGGUGGAGAAGCAGGGCACGUGCAUCCAUUUCCAGCUCGGCAUUCCCG GCGUCCGGCCGGCCGGCUGCCGCGGCCUUGCCUGGCUGGGUAGUGCGUCGCUCG AUCCGGCCGUGCGCCGGCGGCCGGCCCUUGCAUGCAUGUGCCUUUCUUCUCCAC CGUGCACA, an RNA fragment encoding a hairpin motif, the fragment arising from Triticum;

SEQ ID NO: 1151 is CACGUCUUGAGGGGAAUGCAGUGUGGUCCAAGGAGAUGAUAUAUCACUUCACCA UACUCAUAUCUUCACAAACUCAUCAGAUUCAGAUGUAAUAAUAAUUUGUAAUA UAUGCAUAUUAUGUCUCCUCGGACCAGGCUUCAUUCCCUUCAAUUACAG, an RNA fragment encoding a hairpin motif, the fragment arising from Glycine;

SEQ ID NO: 1152 is UGUCUUUUGAGGGGAAUGUUGUCUGGCUCGAGGACCCUUCUUCAUCUUGAUCUU GUGUAGACUACUAUGCUUGUGGUCAAGGAAUACAUAGUGUUGUCGGACCAGGC UUCAUUCCCCCCAAUUAUAU, an RNA fragment encoding a hairpin motif, the fragment arising from Glycine;

SEQ ID NO: 1153 is UGGGGUUGAUGGGAAUGUUGUUUGGCUCGAGGUAACUAUGCAUGGUCUUAAUU UUGUUCAUCUUUGAAGCUUUAAUUUAUGGGUUUCGAUCUCUUUGAUCCCUUGA AACAAAGAAAGCUUUAAAGGUUGGAUUUUGAGGCUUUCUCGGACCAGGCUUCA UUCCCGUAAACCUUA, an RNA fragment encoding a hairpin motif, the fragment arising from Glycine;

SEQ ID NO:1154is AGGUGUUGAGGGGAAUGUUGGCUGGCUCGAGGCUUUUCAAAGAGGAGGUUCUC ACUGGCAAGAACUAUAAGGCUUCGGACCAGGCUUCAUUCCCCUCAAAAAU, an RNA fragment encoding a hairpin motif, the fragment arising from Glycine;

SEQ ID NO: 1155 is UGAGGUUGAGAGGAAUGUUGUCUGGCUCGAGGUCAUGGAGGAGGAGGAGGAGU AGAGUACUGAGAUCAGUGAAAGUUUCCAAUGGAAAUUUACCCUCUUACACAAA AAAAUGAUUCUCGGACCAGGCUUCAUUCCCCCCACCCAAC, an RNA fragment encoding a hairpin motif, the fragment arising from Glycine;

SEQ ID NO: 1156 is UAUCUUUUGAGGGGAAUGUUGUCUGGUGCGAGGCCACCAACUAGAUCCAUGGA AUCCUUCUUUAUAUAUUAUACAGAUCUUUUCUUUUGAAGGGUUUUGACCAUUU UGAUUUUGUUUGAUUUAAGGUUAAGAGGUGGAUCUUGCGUUAGUGUCGUCGGA CCAGGCUUCAUCCCCCCCAAUUGUUU, an RNA fragment encoding a hairpin motif, the fragment arising from Hedyotis;

SEQ ID NO: 1157 is AUGGUUGUCGAGGGGAAUGACNCCGGGUCCNAAAGAGAGACNCUCGCAUGGCGU GCGCGUGGUGCGUUUCGGACCAGGCUUCAUUCCCCAUGACUCCAUC, an RNA fragment encoding a hairpin motif, the fragment arising from Hordeum;

SEQ ID NO: 1158 is GGAGUUUGAGGGGGAUGUUGGCUGGCUCGAUGCACUUACUUAUCAUCUUCCUCA AAAACAUGCGACAUAUACAUACAUAUGGAAGAUCAUAUAUCUAUAAAUAUAUA UUUGUCUUCCAUAUCCCAUAUAUAUAUGUUGAUGAUGGUGGAGUGAUGGCAUC GGACCAGGCUUCAUUCCUCCCAAAACAC, an RNA fragment encoding a hairpin motif, the fragment arising from Ipomoea;

SEQ ID NO: 1159 is GUUAGGUUGAGAGGAACGUUGUCUGGCUCGAGGUGAUGGAGAUGGAAGAGUAC UCUCUACUCACUCAUCACUAACUUUCAAUCUCGGACCAGGCUUCAUUCCCCCCA GCAAACU, an RNA fragment encoding a hairpin motif, the fragment arising from Medicago;

SEQ ID NO: 1160 is CUUAUUUGAGGGGAAUGUUGGCUGGCUCGAGGCUUUUCAGUUUCACAAAGGAA GUUCAGUCUUAAUUGUAUGAACUAUAAGGCUUCGGACCAGGCUUCAUUCCCCUC AAAAUCU, an RNA fragment encoding a hairpin motif, the fragment arising from Medicago;

SEQ ID NO: 1161 is UAUUUUUUGAGGGGAAUGUUGUCUGGCUCGAGGACGCUUUCUUCUCGAUCUAA UGCAAAUUUGUGGUCAUGGAUUGUAAAGUAUUCUCGGACCAGGCUUCAUUCCCC CCAAUUAUAU, an RNA fragment encoding a hairpin motif, the fragment arising from Medicago;

SEQ ID NO: 1162 is AGGAGGUGUUUGGAAUGAGGUUUGUUCCAAGAUCAUCACACAUCAUGUUUCUU CUUCCCUUUCAAUAAUUCUUGAUUAAUUUAUGUUCAUACAUCUAUAUAUAUAU CUAUAUAUAUUAUCAAGUCAUUACAUGCAUGGGAUGAGAUAUUGAGAGUGAUC UCGGACCAGGCUUCAUUCCUCAC, an RNA fragment encoding a hairpin motif, the fragment arising from Medicago;

SEQ ID NO: 1163 is UUAGGUUAAGGGGAUUGUUGUCUGGUUCAAGGUCUCCACAUUGUGCAAAAUGU UCAUUCAUGGAGGCACAGGAUGCUUGGUGAUCUCGGACCAGGCUUCAAUCCCUU UAACCAGG, an RNA fragment encoding a hairpin motif, the fragment arising from Oryza;

SEQ ID NO: 1164 is UUGCUUCUGAGUGGAAUGUUGUCUGGUUCAAGGUCUCGCUUGUGAUUUAAGGA UGAUUUGUGCAUGCGUAAUUUUUAUUCCUUGAAUCUAUGAGAUCUCGGACCAG GCUUCAUUCCCCUCAGCAAUAG, an RNA fragment encoding a hairpin motif, the fragment arising from Sorghum;

SEQ ID NO: 1165 is CUUACUUUGAGGGGAAUGUUGUCUGGCUCGAGGUGCAGAAACAUGCAGAUCUC AUCGGUCUAGGUUCUUGUCGAUCUCGGACCAGGCUUCAUUCCCCUCAAGUGGAG, an RNA fragment encoding a hairpin motif, the fragment arising from Zea;

SEQ ID NO: 1166 is UGCAGCAGUUGAAGCUGCCAGCAUGAUCUGAGUUUACCUUCUAUUGGUAAGAAC AGAUCAUGUGGCUGCUUCACCUGUUGAA, an RNA fragment encoding a hairpin motif, the fragment arising from Glycine;

SEQ ID NO: 1167 is AAGGAAAAAGUGAAGCUGCCAGCAUGAUCUAGCUUUGGUUAGUGGGAGCGAGA UAGUGCUAACCCUCACUAGGUCAUGCUGUGCUAGCCUCACUCCUUCCUA, an RNA fragment encoding a hairpin motif, the fragment arising from Glycine;

SEQ ID NO: 1168 is CAUUAGGAGCUGAAGCUGCCAGCAUGAUCUGAUGAGUGCUUAUUAGGUGAGGG CAGAAUUGACUGCCAAAACAAAGAUCAGAUCAUGCUGUGCAGUUUCAUCUGCUU GUG, an RNA fragment encoding a hairpin motif, the fragment arising from Oryza;

SEQ ID NO: 1169 is ACAAGUUGGUGAAGCUGCCAGCAUGAUCUGAUGAUGAUGAUGAUCCACCUCUCU CAUCUGUGUUCUUGAUUAAUUACGGAUCAAUCGAUCAGGUCAUGCUGUAGUUU CAUCUGCUGGU, an RNA fragment encoding a hairpin motif, the fragment arising from Oryza;

SEQ ID NO: 1170 is ACUACCAGUUGAAGCUGCCAGCAUGAUCUUAACUUCCCUCACUUGGUUGAGGAG AGAUCAGAUCAUGUGGCAGUUUCACCUAGUUGU, an RNA fragment encoding a hairpin motif, the fragment arising from Phaseolus;

SEQ ID NO:1171is ACAAGUUGGUGAAGCUGCCAGCAUGAUCUGAUGGUGGUAUAUAUGAAUAUAUG AUGUCUUUACCUCUGAUCUCUCCCUGACUGUCACGGAUCCAUGAAUCCAGGAUG AGGGGAGGGAAGAAAGAGGGAUAAUGAGCAUCAGGUCAUGCUGUAGUUUCAUC UGCUGGU, an RNA fragment encoding a hairpin motif, the fragment arising from Saccharum;

SEQ ID NO: 1172 is CACAAGUUGGUGAAGCUGCCAGCAUGAUCUGAUGGUGGUAUAUAUGAAUAUAU GAUGUCUUUACCUCUGAUCUCUCCCUGACUGUCACCGAUCCAUGAAUCCAGGAU GAGGGGAGGGAAGAAAGAGGGAUAAUGAGCAUCAGGUCAUGCUGUAGUUUCAU CUGCUGGUG, an RNA fragment encoding a hairpin motif, the fragment arising from Saccharum;

SEQ ID NO: 1173 is ACAAGUUGGUGAAGCUGCCAGCAUGAUCUGAUGGUGGUAUAUAUGAAUAUAUG AUGUCUUUACCUCUGAUCUCUCCCUGACUGUCACGGAUCGAUGAAUCCAGGAUG AGGGGAGGGAAUAAUGAGCAUCAGGUCAUGCUGUAGUUUCAUCUGCUGGU, an RNA fragment encoding a hairpin motif, the fragment arising from Saccharum;

SEQ ID NO: 1174 is CUCUAGUAGCUGAAGCUGCCAGCAUGAUCUGAGGUGUCCACAGCAUAUAUAUGG AAGCAGCUAGCGAUCAGAUCAUGCUGUGCAGUUUCAUCUGCUCGUG, an RNA fragment encoding a hairpin motif, the fragment arising from Zea;

SEQ ID NO: 1175 is GGCUCGGAUUCGCUUGGUGCAGGUCGGGAACCAAUUCGGCUGACACAGCCUCGU GACUUUUAAACCUUUAUUGGUUUGUGAGCAGGGAUUGGAUCCCGCCUUGCAUCA ACUGAAUCGGAUCC, an RNA fragment encoding a hairpin motif, the fragment arising from Arabidopsis;

SEQ ID NO: 1176 is GUCUCUAAUUCGCUUGGUGCAGGUCGGGAACUGCUUCGCUUUUGCCCUUAGAAC ACGCAUACAUGUUUGAGAGUACUGUUAAAGUCUUUAGCCAGCUGGCGCGUUGCG GUUGGCUGGCUGGCUGUGGUCGUGUGGGGAAUAUAGGGCGAAUUGGAUCCCGC CUUGCAUCAACUGAAUCGGAGAC, an RNA fragment encoding a hairpin motif, the fragment arising from Betula;

SEQ ID NO: 1177 is GUCUCUAAUUCGCUUGGUGCAGGUCGGGAACCGGUUUUCGCGCGGAAUGGAGGA GCGGUCGCCGGCGCCGAAUUGGAUCCCGCCUUGCAUCAACUGAAUCGGAGGC, an RNA fragment encoding a hairpin motif, the fragment arising from Glycine;

SEQ ID NO: 1178 is GUCUCCGAUUCGCUUGGUGCAGGUCGGGAACUGCUUCUUCACUCACGGAAAGAA AAAUAUGUGCCUUUACAGAGAAGCAACGUUUUUUAUUUUAUAAUUUUUUGAAC GACGUUUCUGGUGAAUUUAAGUUCAUGCCUUGCAUCAACUGAAUUGGAUGA, an RNA fragment encoding a hairpin motif, the fragment arising from Hedyotis;

SEQ ID NO: 1179 is GCCUCUUAUUCGCUUGGUGCAGGUCGGGACCUCAUUCGCCGGCGCCGGGAAUAA UGCCGGACGAACGACGGCGGUGUUAAUUCUACUAAAGCUGUCACCGACGGAUAG AUGUUUGAUUAGCGGCGAAAUUUGGGUCCUGCCUUGCAUCAACUGAAUUGGAG AC, an RNA fragment encoding a hairpin motif, the fragment arising from Lycopersicon;

SEQ ID NO: 1180 is GCCUCUUAUUCGCUUGGUGCAGGUCGGGACCUCAUUCGGCGGCGCCGGGAAUAA UGCCGGACGAACGACGGCGGUGUUAAUUCUACUAAAGCUGUCACCGACGGAUAG AUGUUUGAUUAGCGGCGAAAUUUGGGUCCUGCCUUGCAUCAACUGAAUUGGAG AC, an RNA fragment encoding a hairpin motif, the fragment arising from Lycopersicon;

SEQ ID NO: 1181 is GCCUCGGGCUCGCUUGGUGCAGAUCGGGACCCGCCGCCGCCGCUGCCGGGGCCG GAUCCCGCCUUGCACCAAGUGAAUCGGAGCC, an RNA fragment encoding a hairpin motif, the fragment arising from Oryza;

SEQ ID NO: 1182 is GUCUCUAAUUCGCUUGGUGCAGGUCGGGAACUGAUUCUGCGAUUUCAUUGCCAG AUGGCUAAACACGAUUGGCUGUGAGGCAAAUUAUAAAAAGAAAGAGAAUUGGA UCCCGCCUUGCAUCAACUGAAUCGGAGAC, an RNA fragment encoding a hairpin motif, the fragment arising from Populus;

SEQ ID NO: 1183 is GUCUCUGAUUCGCUUGGUGCAGGUCGGGAACUGAUUCGGCGAUUUGAUUGCCAG CUGGCUGGACAUGACUGGUUGUUAUGGAAAAAGAAAAGGAAGGAAACAGGAAA AAACAAAGAAUAGCGAAUUGGAUCCCGCCUUGCAUCAACUGAAUCGGAGGC, an RNA fragment encoding a hairpin motif, the fragment arising from Populus;

SEQ ID NO: 1184 is GCCUCGGGCUCGCUUGGUGCAGAUCGGGACCCGCCGCCCGGCCGACGGGACGGA UCCCGCCUUGCACCAAGUGAAUCGGAGCC, an RNA fragment encoding a hairpin motif, the fragment arising from Saccharum;

SEQ ID NO: 1185 is GCCUCAGGCUCGCUUGGUGCAGAUCGGGACCCGCCGCCCGGCCGACGGGACGGA UCCCGCCUUGCACCAAGUGAAUCGGAGCC, an RNA fragment encoding a hairpin motif, the fragment arising from Saccharum;

SEQ ID NO: 1186 is GCCUCUCAUUCGCUUGGUGCAGGUCGGGACCUACCUCGCCGGCACAAUGGCGGU AGCUGACGGCGACGCCAGCGUACCGGUAAAAACUAAUUUUUUACAUGUUGUCUG UGGCGUAGUUUGGGUCCCGCCUUGCAUCAACUGAAUAGGAGAC, an RNA fragment encoding a hairpin motif, the fragment arising from Solanum;

SEQ ID NO: 1187 is GCCUCUCAUUCGCUUGGUGCAGGUCGGGACCUACCUCGCCGGCAACAAUGGCGG UAGCUGACGGCGACGCCAGCGUACCGGUAAAAACUAAUUUUUUACAUGUUGUCU GUGGCGUAGUUUGGGUCCCGCCUUGCAUCAACUGAAUAGGAGAC, an RNA fragment encoding a hairpin motif, the fragment arising from Solanum;

SEQ ID NO: 1188 is GCCUCUCAUUCGCUUGGUGCAGGUCGGGACCUACCUCGCCGGCAACAAUGGCGG UAGCUGACGGCGACGGCAGCUUACCGGUAAAAACUUUUUUUUUUACAUGUCUG UGGCGUAGUUUGGGUCCCGCCUUGCAUCAACUGAAUAGGAGAC, an RNA fragment encoding a hairpin motif, the fragment arising from Solanum;

SEQ ID NO: 1189 is GCCUCGGGCUCGCUUGGUGCAGAUCGGGACCUGCCGCCGUGCUCGGACGGGACA GAUCCCGCCUUGCACCAAGUGAAUCCGAGCC, an RNA fragment encoding a hairpin motif, the fragment arising from Sorghum;

SEQ ID NO: 1190 is GUCUCUAAUUCGCUUGGUGCAGGUCGGGAACCGACUUCGCCGCUCCGGCAGCGC CGGAGGCACGCGGCGGCCUACGAUUGGUUGCUGAGCGAAUUCCGAUCCCGCCUU GCAUCAACUGAAUCGGAGAC, an RNA fragment encoding a hairpin motif, the fragment arising from Vitis;

SEQ ID NO: 1191 is GUCUCGGGCUCQGCUGGUGCAGAUCGGGACCCGCCGCCCGGCCGACGGGACGGA UCCCGCCUUGCAUCAAGUGAAUCGGAGCC, an RNA fragment encoding a hairpin motif, the fragment arising from Zea;

SEQ ID NO: 1192 is GCCUCGGGCUCGCUUGGUGCAGAUCGGGACCCGCCGCCCGGCCGACGGGACGGA UCCCGCCUUGCACCAAGUGAAUCGGAGCC, an RNA fragment encoding a hairpin motif, the fragment arising from Zea;

SEQ ID NO: 1193 is GCCUCGGGCUCGCUUGGUGCAGAUCGGGACCCGCCGCCCGGCCGACGGGACGGA UCCCGCCUUGCACCAAGCGAAUCGGAGCC, an RNA fragment encoding a hairpin motif, the fragment arising from Zea;

SEQ ID NO: 1194 is AGUGUAGUGCAGCCAAGGAUGACUUGCCGGCAUUAGCCAAGUGAAUGAGCAUCA UAUAUAUAUAUAUAUAUAUAUAUAUAUAUAUGACUCAUGUUCUUGUCGGCAAG UUGGCCUUGGCUAUAUUGGACU, an RNA fragment encoding a hairpin motif, the fragment arising from Glycine;

SEQ ID NO: 1195 is AGUGUAGUGCAGCCAAGGAUGACUUGCCGGCAUUAGCCAAGUGAAUGAGCAUCA UAUAUAUAUAUAUAUAUAUAUAUGACUCAUGUUCUUGUCGGCAAGUUGGCCUU GGCUAUAUUGGACU, an RNA fragment encoding a hairpin motif, the fragment arising from Glycine;

SEQ ID NO: 1196 is AGAGUGAUGCAGCCAAGGAUGACUUGCCGGCGUUAUUAUUUGCUCAUGUUCAU GCUCACCGGUUUCCUUGCCGGCAAGUUGUGUUUGGCUAUGUUUUGCU, an RNA fragment encoding a hairpin motif, the fragment arising from Glycine;

SEQ ID NO: 1197 is GCCAUGGUGCAGCCAAGGAUGACUUGCCGAUCGAUCGAUCUAUCUAUGAAGCUA AGCUAGCUGGCCAUGGAUCCAUCCAUCAAUUGGCAAGUUGUUCUUGGCUACAUC UUGGC, an RNA fragment encoding a hairpin motif, the fragment arising from Oryza;

SEQ ID NO: 1198 is AACGGGAUGCAGCCAAGGAUGACUUGCCGGCUCCUGGUAUUGGGGGAAUCUCAG CUUUGCUGAAGCGCCUUGGAGUUAGCCGGCAAGUCUGUCCUUGGCUACACCUAG CU, an RNA fragment encoding a hairpin motif, the fragment arising from Oryza;

SEQ ID NO: 1199 is CGACUCAGACUAGCCAAGGAGACUGCCUAUGAACCAGUUCAAGGCUCAUUUUCU GAAUGAUCCUCUACACAAGGACACAGGCAAGUCAUCCUUGGCUACCAGAGCUAC, an RNA fragment encoding a hairpin motif, the fragment arising from Oryza;

SEQ ID NO: 1200 is GCCAUGGUGCAGCCAAGGAUGACUUGCCGAUCGAUCUAUCUAUGAAGCUAAGCU AGCUGGCCAUGGAUCCAUCCAUCAAUUGGCAAGUUUGUUCUUGGCUACAUCUUGG C, an RNA fragment encoding a hairpin motif, the fragment arising from Oryza;

SEQ ID NO: 1201 is CCACUCAGGCUAGCCAAGGAGACUGCCCAUGAACCAGCUUAAAGGAUCAUUAAA UUGCUAAUCCUUCAGGGAGGACACAGGCAAGUCAUCCUUGGCUAUCAGAGAUAA an RNA fragment encoding a hairpin motif, the fragment arising from Oryza;

SEQ ID NO: 1202 is CCACUCAGGCUAGCCAAGGAGACUGCCUAUCUCGGCUCAUCUGAACCAGCCAGG CCACACUGAUCAUGGCACUGCAUCAUUCAGAAGAGCACAAUAGGCAAGUCAUCC UUGGCUACCAGAGGCAG, an RNA fragment encoding a hairpin motif, the fragment arising from Oryza;

SEQ ID NO: 1203 is UUGCCUCUGUUAGCCAAGAAUGGCUUGCCUAUCUCCACUAUUUGGUUCAUCACU GGAACCCACUUGGGGUUCUCCGAUGGUGGAUGAAAUAUGGAAGAUGGUGAGCC UUCAUGGCUAAGAGAGUGAU, an RNA fragment encoding a hairpin motif, the fragment arising from Oryza;

SEQ ID NO: 1204 is AGAUUGAUGCAGCCAAGGAUGACUUGCCGACGACUCGCUUUGCUUUGCUUCC AUCAAUAUAGGCAUAAUCAAGAAGAGAUGAAUCCGUUGGCAGGUUGUUCUUGG CUACAUUUUUCU, an RNA fragment encoding a hairpin motif, the fragment arising from Populus;

SEQ ID NO: 1205 is AGUUUGAUGCAGCCAAGGAUGAUUUGCCGACGACUCAGUUUUUGCUUCCAUAUG GUAGGAGAGAUGAAGAGGUUGGCAGGUUUUCCUUGGCUACAUUUUCCU, an RNA fragment encoding a hairpin motif, the fragment arising from Populus;

SEQ ID NO: 1206 is CCAUCUUCGAUAGCCAAGGAUGAUUUGCCUGUGAAACUCCCUUGGCAGCCGAGC UCUCUGCCACAGAGAGCGGCGUCCGGCGGUUCCAUGGGCAAGUCACCCUGGGCU ACCCGAAGUAC, an RNA fragment encoding a hairpin motif, the fragment arising from Triticum;

SEQ ID NO: 1207 is GAACUAGGUGUAGCCAAGGACAGACUUGCCGACUGAGUUCUAAGCCUCAGCAGC AAGCUGAGACGCCUCCAGGUUCCAGGAGCCGGCAAGUCAUCCUUGGCUGCAUCC CGUUC, an RNA fragment encoding a hairpin motif, the fragment arising from Zea;

SEQ ID NO: 1208 is GAGUCCCUUUGAUAUUGGCCUGGUUCACUCAGAUCUUACCUGACCACACACGUA GAUAUACAUUAUUCUCUCUAGAUUAUCUGAUUGAGCCGCGCCAAUAUCUCAGUA CUCU, an RNA fragment encoding a hairpin motif, the fragment arising from Arabidopsis;

SEQ ID NO: 1209 is GUUCAACGGGAUAUUGGUCCGGUUCAAUAAGAAAGCAAUGCUCAAAAUGUUUU UGGGUCCUGUUUUUUCAUUGAGCCGUGCCAAUAUCACGAACCAC, an RNA fragment encoding a hairpin motif, the fragment arising from Glycine;

SEQ ID NO: 1210 is CAUAAACGAGAUCUUGGUGCGGUUCAAUGGUAACGGUUGUGGUCGUAACAUUA AGACCCCAAUUUUUCGAUUGAGCCGUGCCAAUAUCACGUACUAU, an RNA fragment encoding a hairpin motif, the fragment arising from Hedyotis;

SEQ ID NO: 1211 is GGUCACUAUGAUGUUGGCUCGACUCACUCAGACCACGCCGGAGGGAGCCAUCUG CGGCGGCGGUUCUGAUUGAGCCGUGCCAAUAUCUUAGUGCUC, an RNA fragment encoding a hairpin motif, the fragment arising from Hordeum;

SEQ ID NO: 1212 is GGGAGAGUGCGAUGUUGGCAUGGUUCAAUCAAACCGGGCAAACUUAUGCACUA GCUAAGCAAGAUGCAGGGAUCUGCAGUAUGGUUUUGUUUGGUCUGAUUGAGCC GUGCCAAUAUCACAAGCUUGC, an RNA fragment encoding a hairpin motif, the fragment arising from Oryza;

SEQ ID NO: 1213 is GGUAGCUAUGAUGUUGGCUCGGCUCACUCAGACGGCAUUGGCGUGAUGCAAAGC AUGCAUGCGUGCUUGCUAGCUCACUUGUGUUUCUGAUUGAGCCGUGCCAAUAUC UUAGUGCUC, an RNA fragment encoding a hairpin motif, the fragment arising from Oryza;

SEQ ID NO: 1214 is AGUGAACGCGGUAUUGGUGCGGUUCAAUCAGAGAGCUGGCGCCCCAGGAGGCAA GGGGUUCCUCCCUUCGAUUGAGCCGUGCCAAUAUCACGCGGUUU, an RNA fragment encoding a hairpin motif, the fragment arising from Triticum;

SEQ ID NO: 1215 is UGGUCACUAUGAUGUUGGCUCGACUCACUCAGACCACGCCUGCCGGCCGGCCGU AGCCAUGCAUCUGCAUGCGGUGGUGGCUCUGAUUGAGCCGUGCCAAUAUCUCAG UGCUCU, an RNA fragment encoding a hairpin motif, the fragment arising from Triticum;

SEQ ID NO: 1216 is GAGAGAGUGCGAUGUUGGCAUGGCUCAAUCAACUCGCCGGCCGCGGGUGGCUUA GCUUAUUAAUUCUGCGUUUUUGAUCGAGGUGCGGGCGCAGUGUUUAAUUGAUU GAGCCGUGCCAAUAUCACAACCUUCU, an RNA fragment encoding a hairpin motif, the fragment arising from Zea;

SEQ ID NO:1217is AGAGAGUGCGAUGUUGGCAUGGCUCAAUCAACUCGCCGGCCGCGGGUGGCUUAG CUUAUUAAUUCUGCGCGUUCGAUCGAGGUGCGGGCGCAGUGUUUAAUUGAUUG AGCCGUGCCAAUAUCACAACCUUC, an RNA fragment encoding a hairpin motif, the fragment arising from Zea;

SEQ ID NO: 1218 is UGCUCGCUGUAGCAGCGUCCUCAAGAUUCACAUCCAGUCUAAAGGCAAAAGCAG CAAUUUUUCUUCAUUUUUGCUUGCCUUGGUUUUUGUCAGUGAGAAUCUUGAUG AUGCUGCAACGGCGAUUA, an RNA fragment encoding a hairpin motif, the fragment arising from Citrus;

SEQ ID NO: 1219 is UGUUUGCGGAUGUAGCAUCAUCAAGAUUCACAUGCAAAUGAAGGUGGGUGGGA CUAUGAUGCAAUCCAAGUGCUCUGCCAAUCCAUCGGUCUUUUUGAUGUGAGAAU CUUGAUGAUGCUGCAUCAGCCAUAA, an RNA fragment encoding a hairpin motif, the fragment arising from Glycine;

SEQ ID NO: 1220 is UUAUUUGCGGAUGUAGCAUCAUCAAGAUUCACAUGCAAGCGCAGGUGGUGGGU GGGACUUGAUGCAAUCUAAGUGCUGUGCCAGCCAAGCCAUAGGUCUUUUGGAAC UGAGAAUCUUGAUGAUGCUGCAUCAGCCAUAAA, an RNA fragment encoding a hairpin motif, the fragment arising from Glycine;

SEQ ID NO: 1221 is UGUUUGCAUAUGUGGCAUAAUCAAGAUUCACGUGAAAAGUUGCAAAUUGGUUA UAUAAUUGAUGAAAUUAAUGGCUGGCUAUUUGAAACUCACGAGAAUCUUGAUG AUGCUGCAUCAGCAAUAA, an RNA fragment encoding a hairpin motif, the fragment arising from Lycopersicon;

SEQ ID NO: 1222 is UGCUUGCUAGUGCAGCACCAUCAAGAUUCACAUAGAAAAUAUGGACUAUGAAA UGAAAUAUGCCCAAUUUUUGAAUACAUGAGAAUCUUGAUGAUGCUGCAUUGGC AAAUU, an RNA fragment encoding a hairpin motif, the fragment arising from Solanum;

SEQ ID NO: 1223 is GGGGAAGCAUCCAAAGGGAUCGCAUUGAUCCUUCAUCGCUCUCGCUCGCUUCCA UGGCGGUCGUCGUCUACAAGCAGCUUGACGGAUCAUGCGAUCCUUUUGGAGGCU UCCUC, an RNA fragment encoding a hairpin motif, the fragment arising from Oryza;

SEQ ID NO: 1224 is GUGGAGGACUCCAAAGGGAUCGCAUUGAUCUGGCUAGCUAUCUCGAUCGAUCGC CUCAUCGAUCGACGACGACGUGCGUGAUCGAUCAGUGCAAUCCCUUUGGAAUUU UCCUC, an RNA fragment encoding a hairpin motif, the fragment arising from Oryza;

SEQ ID NO: 1225 is UUGGAGUGUUCCAAAGGGAUCGCAUUGAUCUAAUGACUUUCGAUGUCUAUAUG AUGUUAAUGUUUAGUCAUUUCAUUGGAUCAUGCGAUCCCUUAGGAAUUUUCCA G, an RNA fragment encoding a hairpin motif, the fragment arising from Populus;

SEQ ID NO: 1226 is CAGAGUUUCUUGGCAUUCUGUCCACCUCCACUUCUUGGCCCUAUCUACGUACUC GGAGGUGGAUAUACUGCCAAUAGAGCUGU, an RNA fragment encoding a hairpin motif, the fragment arising from Glycine;

SEQ ID NO: 1227 is CAGAGUUUAUUGGCAUUCUGUCCACCUCCACUUCCUACUCUCUCUCUGAGCCAC AUGUUCGUGAAGUUGGAGGUGGGCAUACUGUCAACUGAGUUCU, an RNA fragment encoding a hairpin motif, the fragment arising from Glycine;

SEQ ID NO: 1228 is CAGAGUUUCUUGGCAUUCUGUCCACCUCCACUUUCAUCCUCCUGUUUCUGUUAA UGGAUCUCUCUCACCUAAUAUGUGGAGGUGGGCAUACUGCCAACAGAGCUGU, an RNA fragment encoding a hairpin motif, the fragment arising from Robinia;

SEQ ID NO: 1229 is GUUGUCUCUUGGAGUUCCUCUGAACGCUUCAUGUGAUUGGCUAGUUAUAGGCCU UUGAUGAAGUGUUUGGGGGAACUCUUAGGUUCAAC, an RNA fragment encoding a hairpin motif, the fragment arising from Glycine;

SEQ ID NO: 1230 is UAUUAUCGUGAGUUCCCUUCAAGCACUUCACAUGGCCCUAUUUCAAUGUCUAAU AUGUGAAGUGUUUGGGGGAACUCUUGGUAUCG, an RNA fragment encoding a hairpin motif, the fragment arising from Oryza;

SEQ ID NO: 1231 is CAUUGUCGUGAGUUCCCUUCAAGCACUUCACGUGGCACUAUUUCAAUGCGUACC GUGUGAAGUGUUUGGGGGAACUCUUGGCAUCC, an RNA fragment encoding a hairpin motif, the fragment arising from Oryza;

SEQ ID NO: 1232 is UAUUACUAUGAGUUCUCUUUAAGCACUUCAUACGACACCAUUAUUGUUAGGGU UGUUAUGAAGUGUUUGGAGGAACUCUCAGUGCCA, an RNA fragment encoding a hairpin motif, the fragment arising from Oryza;

SEQ ID NO: 1233 is UUGUCCACUGGAGUUCUCCUCAAUCCACUUCAGUAGAUAGCUAUGGCUAGGCCU CAUUGCAUUGCACUGUUACAUAACUGUGAUCAUGGGGCCAAAAGCUAGCUAUGU ACAGUGAAGUGCUUGGGGGAACUCCAGUUGACAC, an RNA fragment encoding a hairpin motif, the fragment arising from Oryza;

SEQ ID NO: 1234 is AGUCCCUAGGAGUUCCUUUCAAGCACUUUACGACACACCGUAUUGAGAGUUGUC GUGAAGUGUUUGGGGGAACUCUUAGUGUCG, an RNA fragment encoding a hairpin motif, the fragment arising from Oryza;

SEQ ID NO: 1235 is UAUUAUCAAGAGUUCUCUUUAAGCACUUCAUACGACACCAUUAUUAUAGGGU UGUUGUGAAGUGUUUGGAGGAACUCUCAGUGUCA, an RNA fragment encoding a hairpin motif, the fragment arising from Oryza;

SEQ ID NO: 1236 is UAUUGUCGUGAGUUCCAUUCAAGCACUUCACGUGGCACUAUCUCAAUGCCUACU AUGUGAAGUGUUUGGGGGAACUCUCGGUAUCA, an RNA fragment encoding a hairpin motif, the fragment arising from Oryza;

SEQ ID NO: 1237 is GUCUUAGCAUGGGUUCCUUACAAGCACUUCACUAGGCAUUGAAAUGCCAAUGUG AAGUGUUUGGAGGGACUCUUAGUGGCAU, an RNA fragment encoding a hairpin motif, the fragment arising from Triticum;

SEQ ID NO: 1238 is UCUUACCAUGGGUUCCUUGCAAGCACUUCAUGAGGCAUUAUUUGAGAUGCCACU AUGAAGUGUUUGGGGGAACUCUUGGUGAUG, an RNA fragment encoding a hairpin motif, the fragment arising from Triticum;

SEQ ID NO: 1239 is GGUUAUAUUUUUCCACAGCUUUCUUGAACUUUCUUUUUCAUUUCCCUUAUUUUA SAGCGAAAUUAAAUAACUAAAAAUCUCUAACAUUUAACACUCUASAAAAAAAAA GCUCAASAAAGCUGUGGGAAAACAUGACA, an RNA fragment encoding a hairpin motif, the fragment arising from Brassica;

SEQ ID NO: 1240 is GUCAUGCUUUUCCACAGCUUUCUUGAACUUCUUAUGCAUCUUAUAUCUCUCCAC CUCCAGGAUUUUAAGCCCUAGAAGCUCAAGAAAGCUGUGGGAGAAUAUGGC, an RNA fragment encoding a hairpin motif, the fragment arising from Glycine;

SEQ ID NO: 1241 is UUUGUAUUCUUCCACAGCUUUCUUGAACUGCAUCCAAAGAGUUCCUUUGCAUGC AUGCCAUGGCACUCUUACUCCCAAAUCUUGUUUUGCGGUUCAAUAAAGCUGUGG GAAGAUACAGA, an RNA fragment encoding a hairpin motif, the fragment arising from Glycine;

SEQ ID NO: 1242 is UUUGUAUUCUUCCACAGCUUUCUUGAACUGCAUCUUCCCUAAUUUUUUUAUCUU UUGUUUUUCCUCUCUCAGAUCCAAUUUUUCUUUGGUAAAUACUUAUAAUUUA UCAAAAAAAAAAAAGAUGGAUUUUUGGGAAUUGAAAGAUGGAAAAUUUUAAUU UGUGGGUUGCGGUUCAAUAAAGCUGUGCGAAGAUACAAA, an RNA fragment encoding a hairpin motif, the fragment arising from Mesembryanthemum;

SEQ ID NO: 1243 is UUUGUAUUCUUCCACAGCUUUCUUGAACUGCAUCUUCCCUAAUUUUUUUAUCUU UUGUUUUUCCUCUCUCAGAUCCAAUUUUUUCUUUGGUAAAUACUUAUAAUUUA UCAAAAAAAAAAAAAGAUGGAUUUUUGGGAAUUGAAAGAUGGAAAAUUUUAAU UUGUGGGUUGCGGUUCAAUAAAGCUGUGGGAAGAUACAAA, an RNA fragment encoding a hairpin motif, the fragment arising from Mesembryanthemum;

SEQ ID NO: 1244 is UUUGUAUUCUUCCUCAGCUUUCUUGAACUGCAUCUUCCCUAAUUUUUUAUCUU UUGUUUUUCCUCUCUCAGAUCCAAUUUUUUCUUUGGUAAAUACUUAUAAUUUA UCAAAAAAAAAAAAAGAUGGAUUUUUGGGAAUUGAAAGAUGGAAAAUUUUAAU UUGUGGGUUGCGGUUCAAUAAAGCUGUGGGAAGAUACAAAU, an RNA fragment encoding a hairpin motif, the fragment arising from Mesembryanthemum;

SEQ ID NO: 1245 is CGCCAUAUUUUCCCACAGCUUUCUUGAACUUUCCCAAUGAUGGUUUGUUUCUCA CUAGAAAGAAAAAAAAAGAAGAAAAGAACCGGAAAGUUCAAGAAAGCUGUGGA AAAGCAUGGCA, an RNA fragment encoding a hairpin motif, the fragment arising from Mesembryanthemum;

SEQ ID NO: 1246 is UUUGUGAUCUUCCACAGCUUUCUUGAACUGCACGCAUGAUGAAUAAUCCCUUUG GUUAAUUGUGAUCUGGUCUCUGAGAGAUCGUAGCUAGACUCGAUCGGUUGCAU UGGCAUCAGAGAGAGCAGUUCAAUAAAGCUGUGGGAAAUUGCAGA, an RNA fragment encoding a hairpin motif, the fragment arising from Oryza;

SEQ ID NO: 1247 is UUUGUAUUCUUCCACAGCUUUCUUGAACUGCACCUAUUAGAUUUAUGUUGAUG UUGUUGUGCGAUUUGCCAUGACCAUAUGACAUUGUAUUCAUUUUUGCUGCGGU UCAAUAAAGCUGUGGGAAGAUACAAA, an RNA fragment encoding a hairpin motif, the fragment arising from Populus;

SEQ ID NO: 1248 is UUUGUAUUCUUCCACAGCUUUCUUGAACUGCACCUAUUAGAUUUAUGUUGAUG UUGUUGUGCGAUAUGCCAUGACCAUAUGACAUUGUAUUCAUUUUUGCUGCGGU UCAAUAAAGCUGUGGGAAGAUACAAA, an RNA fragment encoding a hairpin motif, the fragment arising from Populus;

SEQ ID NO: 1249 is UUUGUAUUCUUCCACAGCUUUCUUGAACUGCAUCCAUGAGAUCGAUCGAUCUUU GCAUGUGAGGCUGCAGUCACUCACUCACUCUCUCUCUAACUGGCUUGCGGUUCA AUAAAGCUGUGGGAAGAUACAGA, an RNA fragment encoding a hairpin motif, the fragment arising from Prunus;

SEQ ID NO: 1250 is UUUGUAUUCUUCCACAGCUUUCUUGAACUGCAUCCAUGAGAUCGAUCGAUCUUU GCAUGUGAUGCUGCAGUCACUCACUCACUCUCUCUCUAACUGGCUUGCGGGUCA AUAAAGCUGUGGGAAGAUACAGA, an RNA fragment encoding a hairpin motif, the fragment arising from Prunus;

SEQ ID NO: 1251 is UUUGUGAUCUUCCACAGCUUUCUUGAACUGCAUCUCUAAGAGGAGCAGCUCGAA GCCUCGAACUCUACCUGCAUGAGCAGGUGCAGUUCAAUAAAGCUGUGGGAAACU GCAGA, an RNA fragment encoding a hairpin motif, the fragment arising from Saccharum;

SEQ ID NO: 1252 is GUCAUGCUUUUCCACAGCUUUCUUGAACUUCUUCUUGCUAAAUUUUGAUCUCUA AAUUGAUAAUUUUGAGAUGAAAUUUUUGAAGCUAUGAAAGUCCAAGAAAGCUG UGGGAAAAGAUGGC, an RNA fragment encoding a hairpin motif, the fragment arising from Solanum;

SEQ ID NO: 1253 is CUUUGUGAUCUUCCACAGCUUUCUUGAACUGCAUCUUUCAGAGGAGCGGCAGUU UCAACUCCUCCACCCGCAUCAGCAGGUGCAUGCAGUUCAAUAAAGCUGUGGGAA ACUGGAAAG, an RNA fragment encoding a hairpin motif, the fragment arising from Zea;

SEQ ID NO: 1254 is CCUGCCAUCUUCCACAGCUUUCUUGAACUGCAUCAUGCAUGCAGCAGGCUGUGC UGUGGACCUGAUCGAGUUUCAAUUGAUCCAAGCAAGCAAGAGGGCAGUUCAAU AAAGCUGUGGGAAAUUGCAGA, an RNA fragment encoding a hairpin motif, the fragment arising from Zea;

SEQ ID NO: 1255 is GCAGAGGUGCCGUUGAGUGCAGCGUUGAUGAACCGUCCGGCCAUGGCCCGUCCG CCUCCACCGAGGCCGGAGCGGUUCACCGGCGCUGCACGCAAUGACGCCUCUGC, an RNA fragment encoding a hairpin motif, the fragment arising from Hordeum;

SEQ ID NO: 1256 is GCAQAGGUGCCGUUGAGUGCAGCGUUGAUGAACCGUCCGGCCAUGGNCCGUCCG CCUCCACCGAGGCCGGAGCGGUUCACCGGCGCUGCACGCAAUGACGCCUCUGC, an RNA fragment encoding a hairpin motif, the fragment arising from Hordeum;

SEQ ID NO: 1257 is GUGAACCCCAGAGGAGUGAACCUGAGAACAGAGGGUGGCGUUGGCUUAAAUUU GAUTJUGCAUUGCUGCUGCCUGCUCCUGCCAUACAUUAAAUCAAAAAGAUUGUAU GUGGCCAACGCACCUUGUGUUCUCAGGUCACCCCUUUGGGAAUUCA, an RNA fragment encoding a hairpin motif, the fragment arising from Citrus;

SEQ ID NO: 1258 is AGGAAUUCUACAGGGUCGUCCUGAGACCACAUGAAACAGAUUCAAAAUACAAGC AUAUUUGCUUGUGACCUUUUGUGACUCAGUUCAUGUGUUCUCAGGUCGCCCCUG CUGAACUUUU, an RNA fragment encoding a hairpin motif, the fragment arising from Glycine;

SEQ ID NO: 1259 is GUUUAUCUCAGAGGAGUGGAUCUGAGAACACAAGGCUGGUUUGCACUGCUAUA UUAUGAUCGAUUGGUAUAAGGUGAAUUUACUUUGUGUUCUCAGGUCACCCCUU UGAGCCAACC, an RNA fragment encoding a hairpin motif, the fragment arising from Glycine;

SEQ ID NO: 1260 is CUCGGAGGAGUGAAUCUGAGAACACAAGGCUGGUUUGCACUGCUAUAUCAUCUA UUGGUAUAAGGUGAAUUUACUUUGUGUUCUCAGGUCACCCCUUUGAGCCAACC, an RNA fragment encoding a hairpin motif, the fragment arising from Glycine;

SEQ ID NO:1261is GACAUCCAACAGGUGUGACAUGAGAACACAUGAUGAUAUACUUAGAUAUUUUC ACGUGUUCUCAGGUCGCCCCUGCUGAAUUUU, an RNA fragment encoding a hairpin motif, the fragment arising from Helianthus;

SEQ ID NO: 1262 is AGGCAUCCAACAGGUGCGACAUGGGAACACAUGUUAAAUGUGCAACAAAUCACA UUCCGCCAUGUGUUCUCAGGUCGCCCCUGCAGGGUUUUU, an RNA fragment encoding a hairpin motif, the fragment arising from Lactuca;

SEQ ID NO: 1263 is AGGCAUCCAACAGGUGCGACAUGGCAACACAUGUUAAAUGUGCAACAAAUCACA UUCCGCCAUGUGUUCUCAGGUCGCCCCUGCAGGGUUUUU, an RNA fragment encoding a hairpin motif, the fragment arising from Lactuca;

SEQ ID NO: 1264 is GUCUAUCUCAAAGGAGUGAGCCUGAGAACACAAGCUGAAUUGGUUUGAAUUGC CAUAUCACAUACUGAUAUCUGGUAUAGGCUUUAUGUUGCUAAUUUAUUUUGUG UUCUCAGGUCACCCCUUUGAGCUGACC, an RNA fragment encoding a hairpin motif, the fragment arising from Lotus;

SEQ ID NO:1265is UUCUAUCUCAGAGGAGUGACACUGAGAACACAAGAUUGAUUAAUCAUAUAAUG UAUUUGGUUGUUACUAGUUGAUUUUGUGUUCUCAGGUCACCCCUUUGAGUCAA CC, an RNA fragment encoding a hairpin motif, the fragment arising from Medicago;

SEQ ID NO: 1266 is UGAUGUUCUACAGGGUCGACAUGAGAGCACAUGAAGCUAUCAUGGUUGUCUAU GUUAUCCAACUCAUGUGUUCUCAGGUCGCCCCUGCUGAAUUUUC, an RNA fragment encoding a hairpin motif, the fragment arising from Medicago;

SEQ ID NO: 1267 is AAGUGUUCAACAGGGGCAACCUGAGAUCACAUAUUGUCAUUUUUCUUUAGUUG UUGAGUCUGGUUCAAUAUCUCACUAUGUGUUCUCAGGUCGCCCCUGUCGAAUUA UU, an RNA fragment encoding a hairpin motif, the fragment arising from Nicotiana;

SEQ ID NO: 1268 is GAGUUCCUACAGGGGCGAGCUGGGAACACACGGUGAUGAGGCGGUCUGGUCUUU CGUGUGUUCUCAGGUCGCCCCUGCCGGGACUC, an RNA fragment encoding a hairpin motif, the fragment arising from Oryza;

SEQ ID NO: 1269 is CAGUUCCGGCGGGGGCGGACUGGGAACACAUGGGAAUGAGAUGAGAUCAUUGC UCGGUCGUGCUGGCCUGGGCCGUCGGCGCGCGUUGAUCUUGCAUGUGUUCUCAG GUCGCCCCCGGAGGGCCUU, an RNA fragment encoding a hairpin motif, the fragment arising from Zea;

SEQ ID NO: 1270 is AAAUCAGCUAUAGGGCUUCUCUUUCUUGGCAGGAAAUUAUCAUGACCAUUCCAU CAUGUGUCUUGCCAAAGGAGAGUUGCCCUGUUGCUGUUUU, an RNA fragment encoding a hairpin motif, the fragment arising from Medicago;

SEQ ID NO: 1271 is UAAAGAAUAACAGGGCUUUAUCCUCCUUUGGCAAACAGAACAUGGAAAUAAAU GCCUGCAUAUUUCUGUUUUGCCAAGGGAGAAUUGCCCUGCCAUUCGAUU, an RNA fragment encoding a hairpin motif, the fragment arising from Populus;

SEQ ID NO: 1272 is UCCAAAGGGAUCGCAUUGAUC, an miRNA sequence arising from A. thaliana;

SEQ ID NO: 1273 is CUAAUUANNNNNNNNNNNNNNNNNNNNNNNNNNGGCAAAUAAAUCACAAAAAU UUGCUUGGUUUUG, an RNA fragment encoding a stem-loop motif, the fragment arising from A. thaliana;

SEQ ID NO: 1274 is AAUUAAGCNNNNNNNNNNNNNNNNNNUUGUUUUUCUUUUCCUUCUCAAUCGAA AGAUGGAAGAAAAACAANNNNNNNNNNNNNNGCUUACUUUUCCG, and RNA fragment encoding a stem-loop motif, the fragment arising from A. thaliana;

SEQ ID NO: 1275 is UCCAAAGGGAUCGCAUUGAUC, an miRNA sequence arising from O. sativa;

SEQ ID NO: 1276 is CUUCAUCGCUCUCGCUCGCUUCCAUGGCGGUCGNNNNNNNNNAGCAGCUU, an RNA fragment encoding a stem-loop motif, the fragment arising from O. sativa;

SEQ ID NO: 1277 is UUGGCAUUCUGUCCACCUCC, an miRNA sequence arising from A. thaliana;

SEQ ID NO: 1278 is UCUCUCUAUAUUUAUGUGUAAUAAGUGUANNNNNNNNNNNNNNNNNNNNNNNG A, an RNA fragment encoding a stem-loop motif, the fragment arising from A. thaliana;

SEQ ID NO: 1279 is UAUACAUAUAUGCAUGUGUAUAUAUAUAUGCGUCUUGUGUGAAA, an RNA fragment encoding a stem-loop motif, the fragment arising from A. thaliana;

SEQ ID NO: 1280 is UUGGCAUUCUGUCCACCUCC, an miRNA sequence arising from O. sativa;

SEQ ID NO: 1281 is UUGUCGAAUCCUCAGAGACAGAAAUCUCAUACCUGUUGAUCUU, an RNA fragment encoding a stem-loop motif, the fragment arising from O. sativa;

SEQ ID NO: 1282 is ACAAAGCUGGAGACAAUGCGAUCCCUUUGGAUGUCUUCUUG, a fragment of the TIR1 gene arising from A. thaliana;

SEQ ID NO: 1283 is UCCAAAGGGAUCGCAUUGAUC, a fragment of miR393a arising from A. thaliana;

SEQ ID NO: 1284 is GGUAGGUACGAAACAAUGCGAUCCCUUUGGAUGUCGUCUUG, a fragment of the At1g12820 gene arising from A. thaliana;

SEQ ID NO: 1285 is-UCCAAAGGGAUCGCAUUGAUC, a fragment of miR393a arising from A. thaliana;

SEQ ID NO: 1286 is AGCAAGUAUGAAACAAUGCGAUCCCUUUGGAUGUCUUCAUG, a fragment of the At3g26810 gene arising from A. thaliana;

SEQ ID NO: 1287 is UCCAAAGGGAUCGCAUUGAUC, a fragment of miR393a arising from A. thaliana;

SEQ ID NO: 1288 is GCCAAGCUAGAGACCAUGCGAUCCCUUUGGAUGUCAUCUUG, a fragment of the At4g03190 gene arising from A. thaliana;

SEQ ID NO: 1289 is UCCAAAGGGAUCGCAUUGAUC, a fragment of miR393a arising from A. thaliana;

SEQ ID NO: 1290 is CUUACCUUUGGGUCAGAGCGAUCCCUUUGGCAAUGGCAAUG, a fragment of the At3g23690gene arising from A. thaliana;

SEQ ID NO: 1291 is UCCAAAGGGAUCGCAUUGAUC, a fragment of miR393a arising from A. thaliana;

SEQ ID NO: 1292 is CUGUUGUGGAAGGAGGUUGACAGAAUGCCAAACAUAUGGUC, a fragment of the At1g27340 arising from A. thaliana;

SEQ ID NO: 1293 is UUUGGCAUUCUGUCCACCUCC, a fragment of miR394a arising from A. thaliana;

SEQ ID NO: 1294 is GAGACAGUCAGAGUUCCUCCAAACACUUCAUUUUAACUCGU, a fragment of the APS4 arising from A. thaliana;

SEQ ID NO: 1295 is CUGAAGUGUUUGGGGGAACUC, a fragment of miR395a arising from A. thaliana;

SEQ ID NO: 1296 is GAGGCCGCCAUCGUUCAAGAAAGCCUGUGGAAGGCCAAAAU, a fragment of the GRL1 arising from A. thaliana;

SEQ ID NO: 1297 is UUCCACAGCUUUCUUGAACUG, a fragment of miR396a arising from A. thaliana;

SEQ ID NO: 1298 is GAGGCCGUCAUCGUUCAAGAAAGCCUGUGGAAGUCCAAUCU, a fragment of the GRL2 arising from A. thaliana;

SEQ ID NO: 1299 is UUCCACAGCUUUCUUGAACUG, a fragment of miR396a arising from A. thaliana;

SEQ ID NO: 1300 is GUGGCCGCAACCGUUCAAGAAAGCCUGUGGAAACUCCAACC, a fragment of the GRL3 arising from A. thaliana;

SEQ ID NO: 1301 is UUCCACAGCUUUCUUGAACUG, a fragment of miR396a arising from A. thaliana;

SEQ ID NO: 1302 is GAGGUCGUCCUCGUUCAAGAAAGCAUGUGGAACCUCCUUAU, a fragment of the GRL7 arising from A. thaliana;

SEQ ID NO: 1303 is UUCCACAGCUUUCUUGAACUG, a fragment of miR396a arising from A. thaliana;

SEQ ID NO: 1304 is AGAGCCGUCCUCGUUCAAGAAAGCAUGUGGAAUCAUCUCAC, a fragment of the GRL8 arising from A. thaliana;

SEQ ID NO: 1305 is UUCCACAGCUUUCUUGAACUG, a fragment of miR396a arising from A. thaliana;

SEQ ID NO: 1306 is GAGGUCGUAAACGUUCAAGAAAGCUUGUGGAAUCUUCUUCU, a fragment of the GRL9 arising from A. thaliana;

SEQ ID NO: 1307 is TUCCACAGCUUUCUUGAACUG, a fragment of miR396a arising from A. thaliana;

SEQ ID NO: 1308 is UACUACGAUUAAUCAAUQCUGCACUCAAUGACGAACUCUUC, a fragment of the At2g29130 arising from A. thaliana;

SEQ ID NO: 1309 is UCAUUGAGUGCAGCGUUGAUG, a fragment of miR397a arising from A. thaliana;

SEQ ID NO: 1310 is UGCUACGACUAGUCAACGCUGCACUUAAUGAAGAACUCUUU, a fragment of the At2g38080 arising from A. thaliana;

SEQ ID NO: 1311 is UCAUUGAGUGCAGCGUUGAUG, a fragment of miR397a arising from A. thaliana;

SEQ ID NO: 1312 is UUCUCAGGCUAAUCAAUGCUGCACUUAAUGACGAGCUCUUU, a fragment of the At2g60020 arising from A. thaliana;

SEQ ID NO: 1313 is UCAUUGAGUGCAGCGUUGAUG, a fragment of miR397a arising from A. thaliana;

SEQ ID NO: 1314 is AUUCUUUCCAAAGGGGUUUCCUGAGAUCACAAAGGCCAAGU, a fragment of the CSD1 arising from A. thaliana;

SEQ ID NO: 1315 is UGUGUUCUCAGGUCACCCCTU, a fragment of miR398a arising from A. thaliana;

SEQ ID NO: 1316 is AGUGCCGUCAUGCGGGUGACCUGGGAAACAUAAAUGCCAAU, a fragment of the CSD2 arising from A. thaliana;

SEQ ID NO: 1317 is UGUGUUCUCAGGUCACCCCUU, a fragment of miR398a arising from A. thaliana;

SEQ ID NO: 1318 is CUAAUCCUUCAAGGUGUGACCUGAGAAUCACAACACAAAAC, a fragment of the At3g15640 arising from A. thaliana; and

SEQ ID NO: 1319 is UGUGUUCUCAGGUCACCCCUU, a fragment of miR398a arising from A. thaliana.

### DETAILED DESCRIPTION

The present invention generally relates to the production and expression of microRNA (miRNA) in plants. In some cases, production and expression of miRNA can be used to at least partially inhibit or alter gene expression in plants. For instance, in some embodiments, a nucleotide sequence, which may encode a sequence substantially complementary to a gene to be inhibited or otherwise altered, may be prepared and inserted into a plant cell. Expression of the nucleotide sequence may cause the formation of precursor miRNA, which may, in turn, be cleaved (for example, with Dicer or other nucleases, including, for example, nucleases associated with RNA interference), to produce an miRNA sequence substantially complementary to the gene. The miRNA sequence may then interact with the gene (e.g., complementary binding) to inhibit the gene. In some cases, the nucleotide sequence may be an isolated nucleotide sequence. Other embodiments of the invention are directed to the precursor miRNA and/or the final miRNA sequence, as well as methods of making, promoting, and use thereof.

The following definitions will aid in the understanding of the invention. As used herein, the term “sample” is used in its broadest sense. In one sense, it can refer to a plant cell or tissue. In another sense, it is meant to include a plant specimen or culture obtained from any source, as well as biological and environmental samples. Biological samples may be obtained from plants and encompass fluids, solids, tissues, and gases. Environmental samples include environmental material such as surface matter, soil, water, industrial samples, etc., e.g., which may contain plants. These examples are not to be construed as limiting the sample types applicable to the present invention.

As used herein, the term “plant” is used in its broadest sense, including, but is not limited to, any species of woody, ornamental or decorative, crop or cereal, fruit or vegetable plant, and algae (e.g., Chlamydomonas reinhardtii). Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia. Still other examples of plants include, but are not limited to, wheat, cauliflower, tomato, tobacco, corn, petunia, trees, etc. As used herein, the term “cereal crop” is used in its broadest sense. The term includes, but is not limited to, any species of grass, or grain plant (e.g., barley, corn, oats, rice, wild rice, rye, wheat, millet, sorghum, triticale, etc.), non-grass plants (e.g., buckwheat flax, legumes or soybeans, etc.). As used herein, the term “crop” or “crop plant” is used in its broadest sense. The term includes, but is not limited to, any species of plant or algae edible by humans or used as a feed for animals or used, or consumed by humans, or any plant or algae used in industry or commerce.

The term “nucleic acid,” as used herein, is given its ordinary meaning as used in the art. Typically, a nucleic acid includes multiple nucleotides. Nucleotides typically are formed from molecules comprising a sugar (e.g. ribose or deoxyribose) linked to a phosphate group and an exchangeable organic base. A sugar and a base (without the phosphate) together form a nucleoside. Examples of suitable organic bases include, but are not limited to various pyrimidines or purines, which may be naturally occurring (e.g., adenosine (“A”), thymidine (“T”), guanosine (“G”), cytidine (“C”), and uridine (“U”)), nucleoside analogs (e.g., 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, O6-methylguanosine, 2-thiocytidine, 2-aminopurine, 2-amino-6-chloropurine, 2,6-diaminopurine, hypoxanthine), chemically or biologically modified bases (e.g., methylated bases), intercalated bases, modified sugars (2′-fluororibose, arabinose, or hexose), modified phosphate moieties (e.g., phosphorothioates or 5′-N-phosphoramidite linkages), and/or other naturally and non-naturally occurring bases substitutable into the nucleic acid, including substituted and unsubstituted aromatic moieties. Other suitable base modifications are well-known to those of skill in the art.

As used herein, terms such as “polynucleotide” or “oligonucleotide” generally refer to a polymer of at least two nucleotides. Typically, an “oligonucleotide” is a polymer having 20 bases or less, and a “polynucleotide” is a polymer having at least 20 bases. Those of ordinary skill in the art will recognize that these terms are not always precisely defined in terms of the number of bases present within the polymer. Polynucleotides where the sugars are predominantly deoxyribose are referred to as DNA or deoxyribonucleic acid, while polynucleotides where the sugars are predominantly ribose are referred to as RNA or ribonucleic acid.

U.S. Provisional Patent Application Ser. No. 60/484,481, filed Jul. 1, 2003, entitled “Micro RNAs in Plants,” by Reinhart, et al., is incorporated herein by reference.

Various aspects of the present invention relate to the discovery of microRNAs (miRNAs) in plants. Plant miRNAs can be processed from a portion of an miRNA transcript (i.e., a precursor miRNA) that can fold into a stable hairpin or stem-loop structure. Typically, a portion of the precursor miRNA is cleaved to produce the final miRNA molecule, as further discussed below. However, the hairpin structures of the precursor miRNAs of the plant miRNAs are typically more variable in size than their animal counterparts. For instance, the hairpin structures may range from about 64 nucleotides to about 303 nucleotides (counting the miRNA residues, those pairing to the miRNA, and any intervening segment(s), but excluding more distal base pairs). In contrast, animal miRNA hairpin structures typically have 60 to 70 nucleotides. Plant miRNAs also generally pair to the opposite arm of their precursor hairpin with fewer mismatches and bulges than do the animal miRNAs.

One aspect of the invention is directed to plant-derived miRNA. As used herein, a “microRNA” or an “miRNA” is given its ordinary meaning in the art. Typically, the miRNA is a RNA molecule derived from genomic loci processed from transcripts that can form local RNA precursor miRNA structures. The mature miRNA usually has 20 to 24 nucleotides, although in some cases, other numbers of nucleotides may be present (for example, between 18 and 26 nucleotides). miRNAs are usually detectable on Northern blots. The miRNA has the potential to pair to flanking genomic sequences, placing the mature miRNA within an imperfect RNA duplex which may be needed for its processing from a longer precursor transcript. In addition, miRNAs are typically derived from a segment of the genome that is distinct from predicted protein-coding regions. Thus far, >150 RNAs that satisfy these criteria have been identified in animals (e.g., lin-4 and let-7 in C. elegans), although none had been previously identified in plants. As used herein, “plant-derived” miRNA is miRNA that is produced using precursor miRNAs expressed naturally in a plant cell. For instance, the miRNA precursor, or at least a portion thereof (for example, a hairpin or stem-loop motif, as further discussed below), can be expressed from a native plant gene.

In some embodiments, the plant-derived miRNA may be isolated, e.g., from plant cells. An “isolated” molecule, as used herein, is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

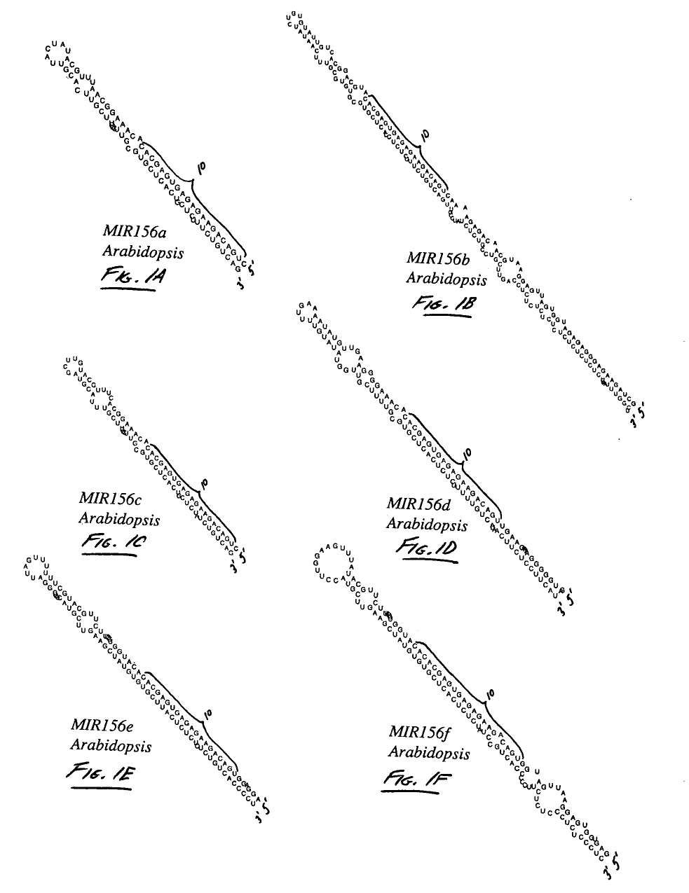

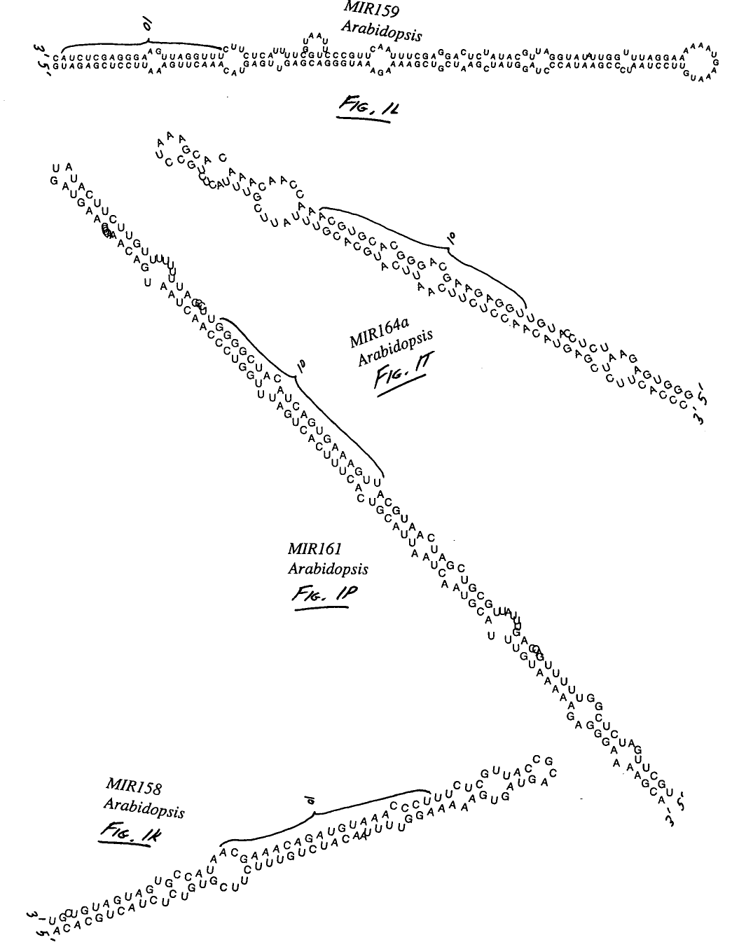

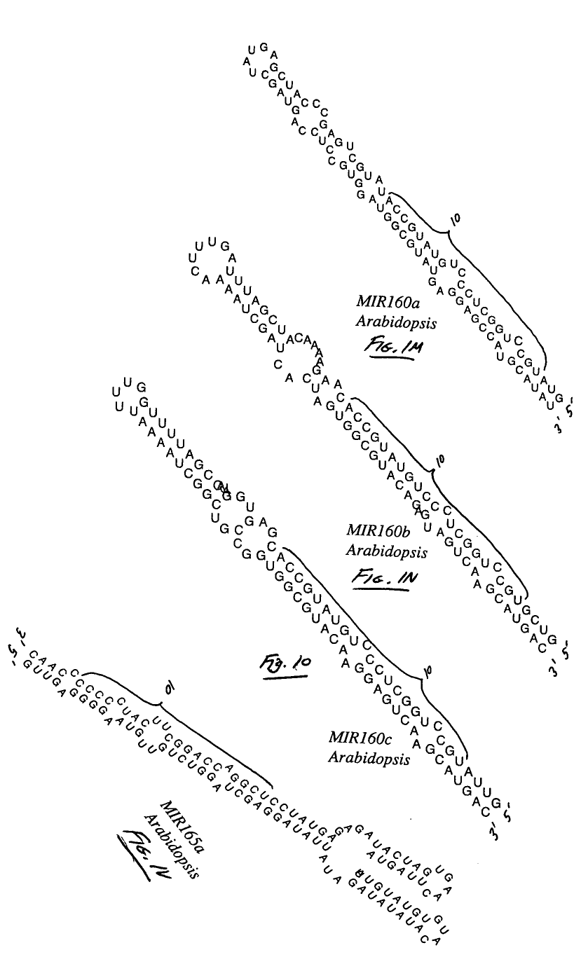

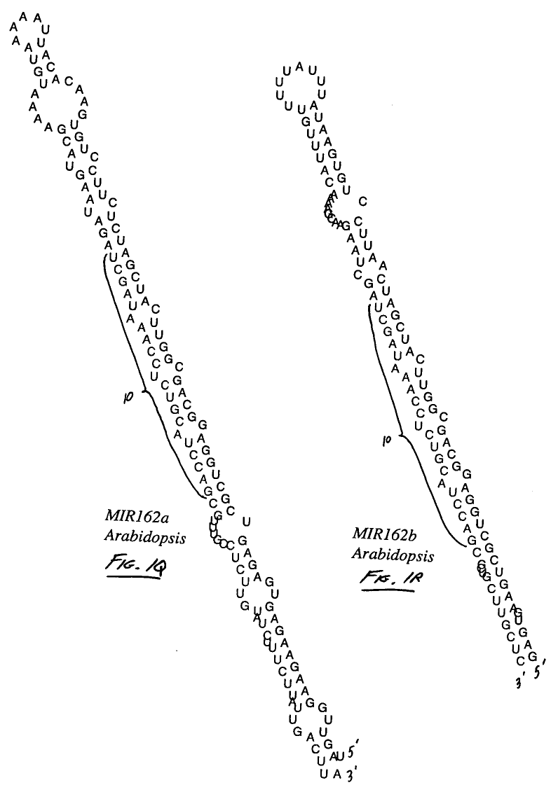

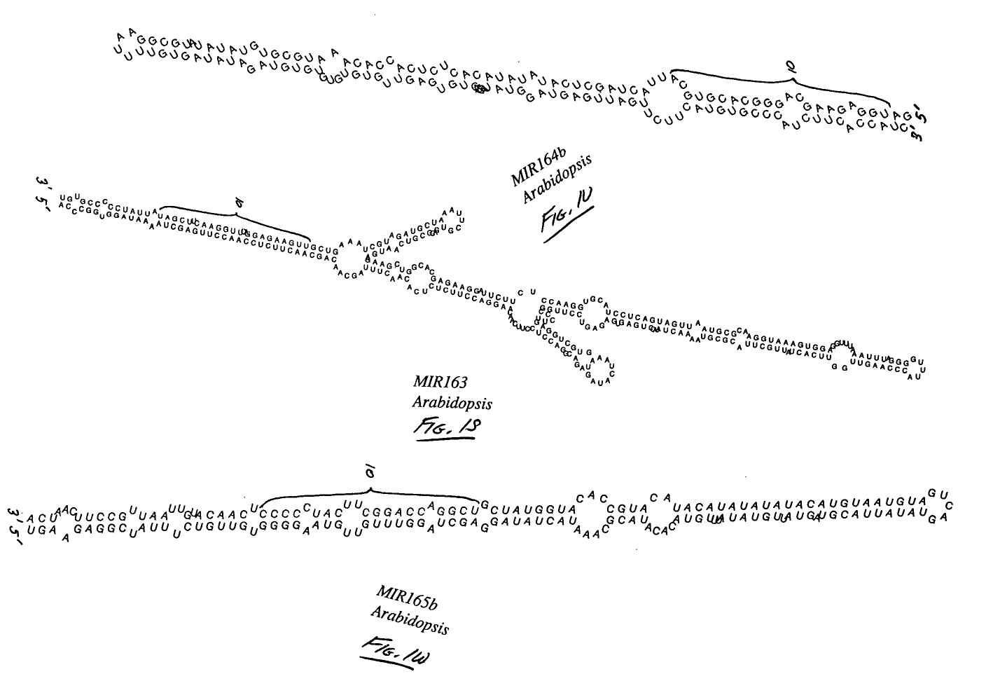

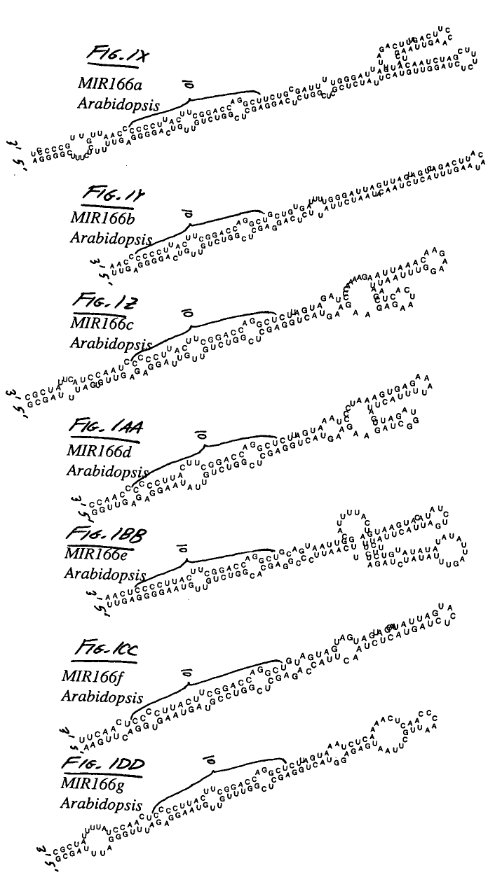

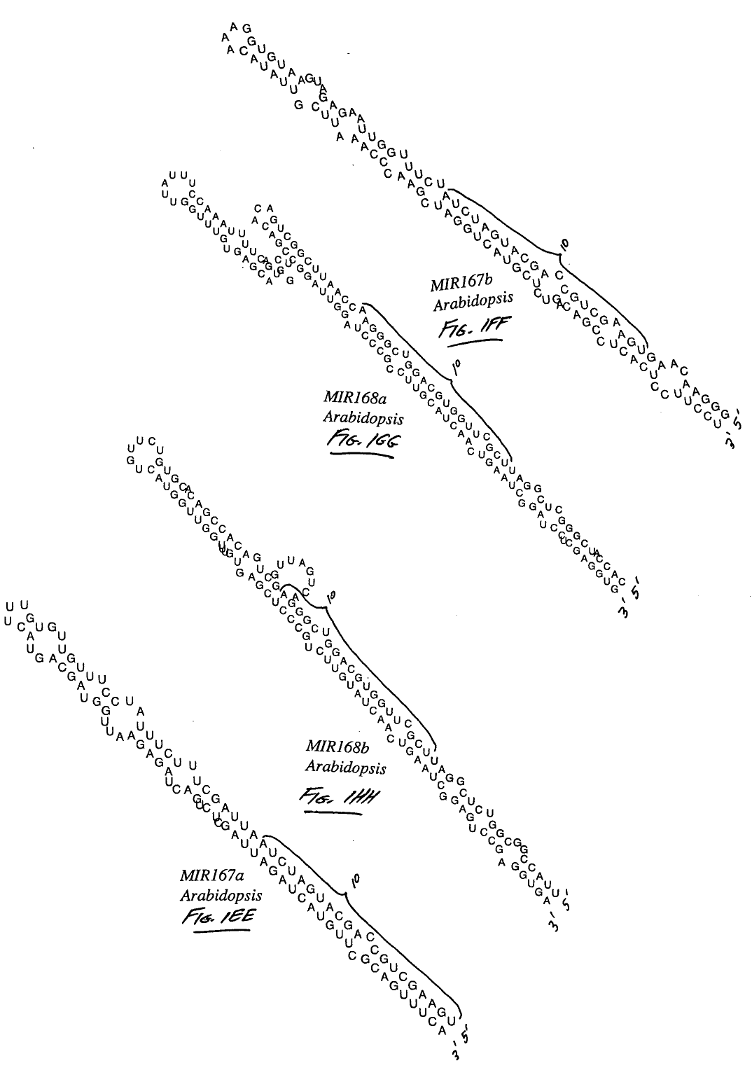

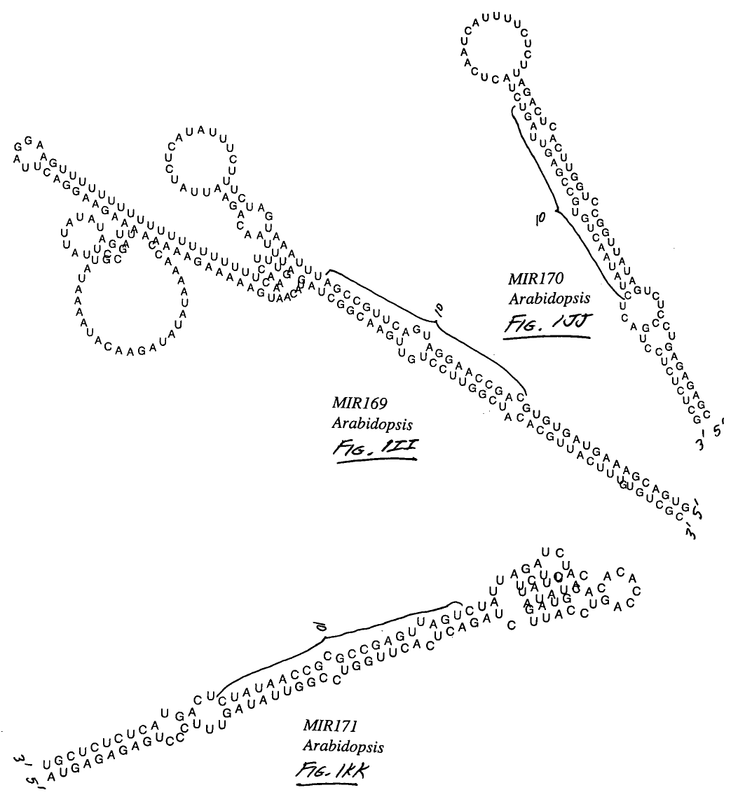

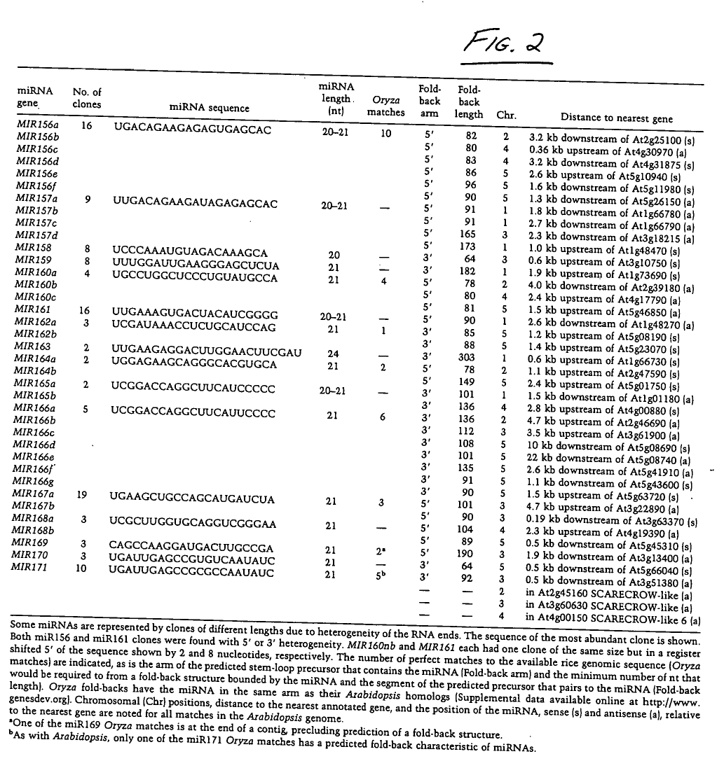

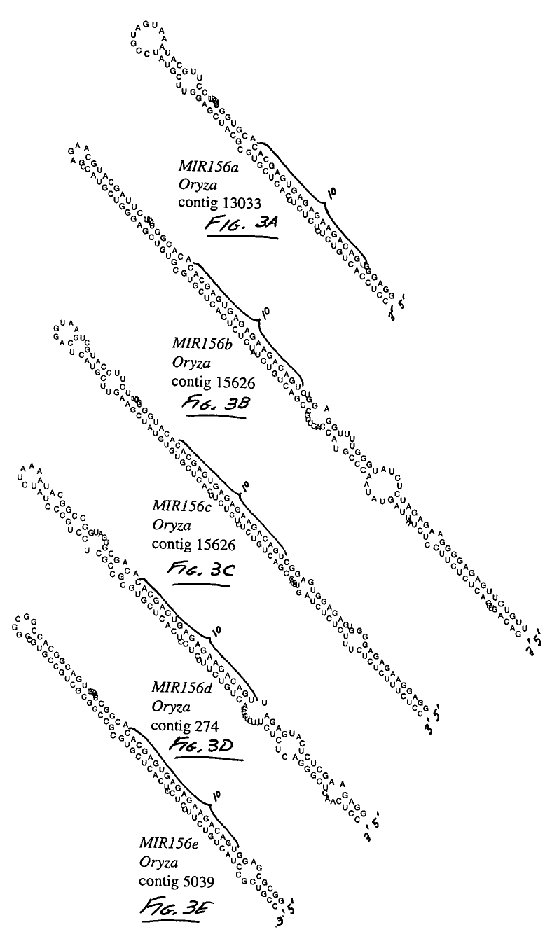

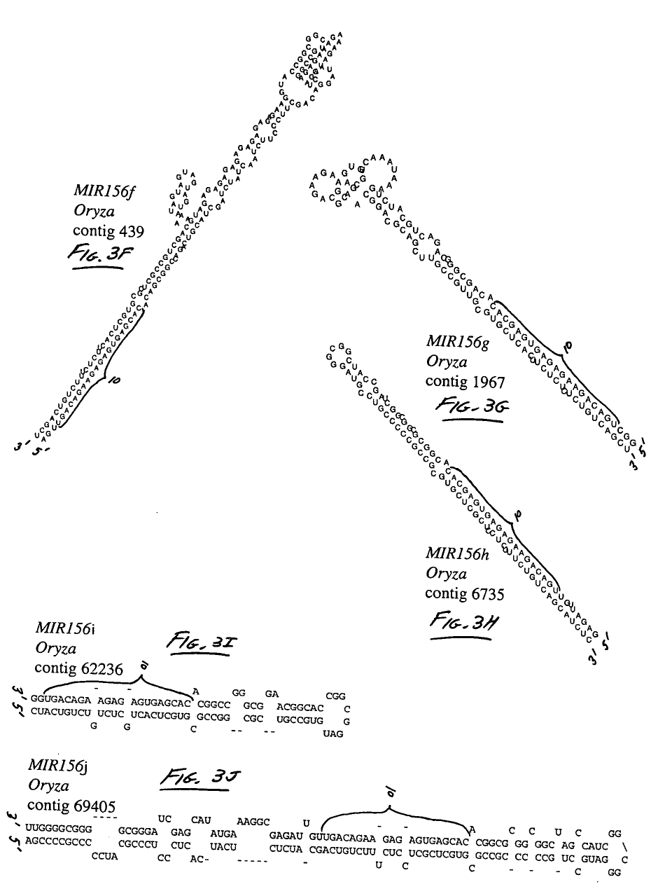

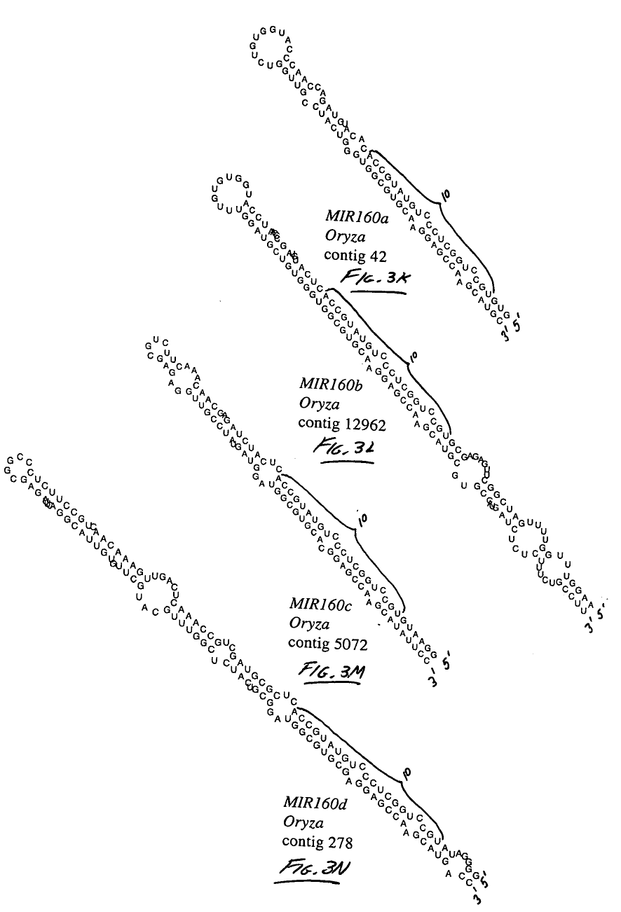

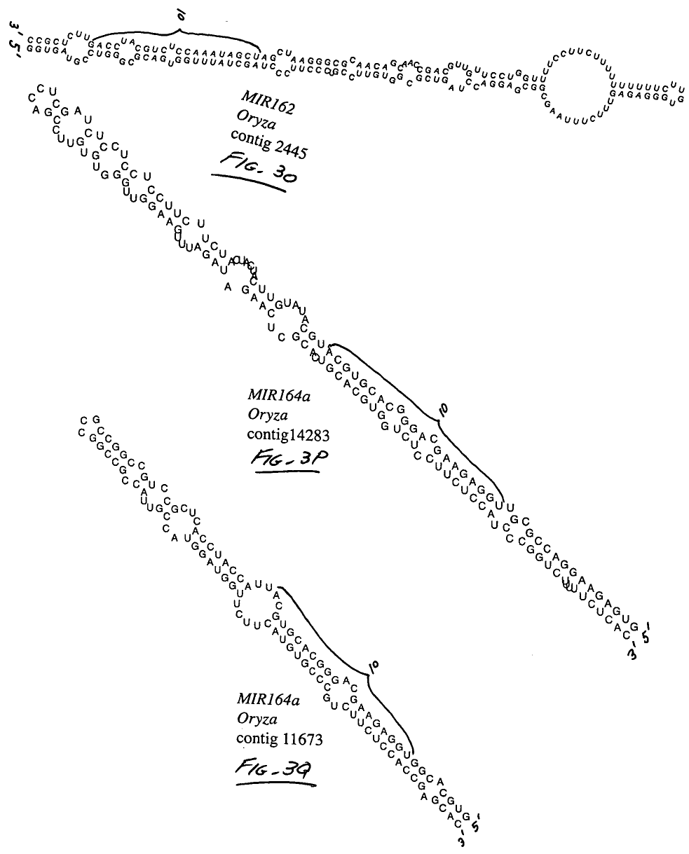

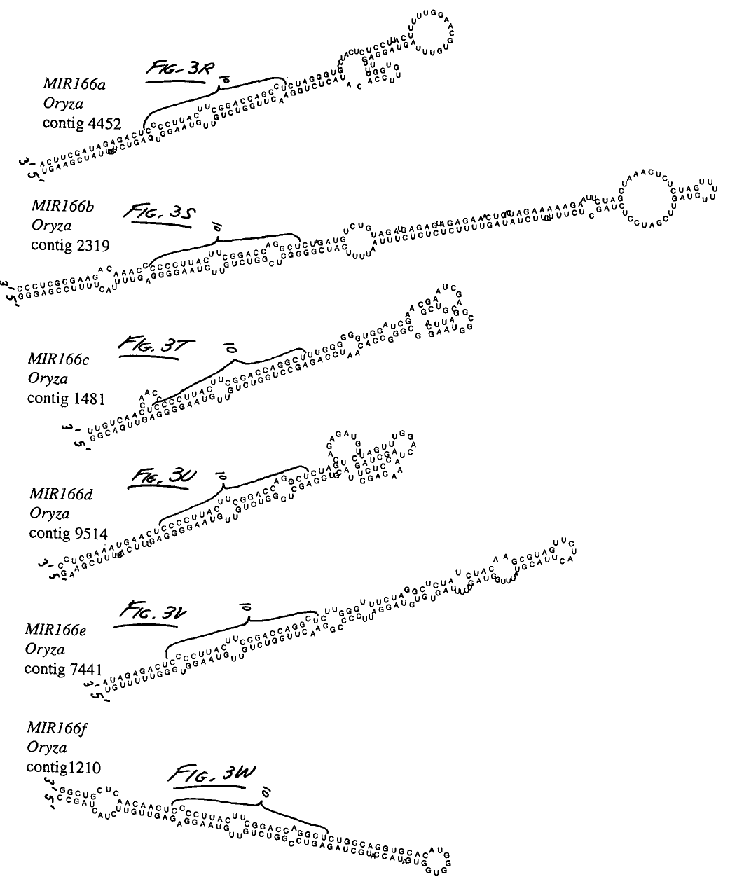

miRNA is typically produced through the processing of precursor miRNA. Thus, another aspect of the invention relates to precursor miRNA that can be processed to produce miRNA in a plant cell. Additionally, the precursor miRNA may be isolated, e.g., from plant cells, according to certain embodiments. One example technique is illustrated in Example 4. As used herein, “precursor miRNA” is generally composed of any type of nucleic acid-based molecules capable of accommodating miRNA sequences and stem-loop motifs incorporating the miRNA sequences. The precursor miRNA may be naturally or artificially generated. Typically, the precursor microRNA molecule is an isolated nucleic acid having a stem-loop structure and a microRNA sequence incorporated therein. The miRNA sequences and the sequences including the stem-loop motifs do not all necessarily have to originate from the same organism. Non-limiting examples of precursor miRNAs include those shown in FIGS. 1A-1KK and FIGS. 3A-3BB. In each sequence, the miRNA portion 10 of each precursor miRNA is indicated as shown. In some embodiments, the primary sequence of the precursor miRNA, exclusive of the miRNA, is derived from natural sequences flanking plant-derived miRNAs.

The precursor miRNA can be cleaved or otherwise processed by the plant cell to produce miRNA substantially complementary to at least a portion of an mRNA sequence encoding a gene. As used herein, “substantially complementary,” in reference to nucleic acids, refers to sequences of nucleotides (which may be on the same nucleic acid molecule or on different molecules) that are sufficiently complementary to be able to interact with each other in a predictable fashion, for example, producing a generally predictable secondary structure, such as a stem-loop motif. In some cases, two sequences of nucleotides that are substantially complementary may be at least about 75% complementary to each other, and in some cases, are at least about 80%, at least about 85%, at least about 90%, at least about 95%, at least about 96%, at least about 97%, at least about 98%, at least about 99%, at least about 99.5%, or 100% complementary to each other. In some cases, two molecules that are sufficiently complementary may have a maximum of 40 mismatches (e.g., where one base of the nucleic acid sequence does not have a complementary partner on the other nucleic acid sequence, for example, due to additions, deletions, substitutions, bulges, etc.), and in other cases, the two molecules may have a maximum of 30 mismatches, 20 mismatches, 10 mismatches, or 7 mismatches. In still other cases, the two sufficiently complementary nucleic acid sequences may have a maximum of 0, 1, 2, 3, 4, 5, or 6 mismatches.

As used herein, a “stem-loop motif” or a “stem-loop structure,” sometimes also referred to as a “hairpin structure,” is given its ordinary meaning in the art, i.e., in reference to a single nucleic acid molecule having a secondary structure that includes a double-stranded region (a “stem” portion) composed of two regions of nucleotides (of the same molecule) forming either side of the double-stranded portion, and at least one “loop” region, comprising uncomplemented nucleotides (i.e., a single-stranded region). The double-stranded portion of the nucleic acid may remain double-stranded even if the two nucleotide regions forming the double-stranded portions are not perfectly complementary to each other, i.e., the two regions are substantially complementary to each other. For example, additions, deletions, substitutions, etc. may occur in one region relative to the other, and in some cases, one region itself may contain stem-loop motifs or other secondary structures that are not found in the complementary region. However, the two regions may be substantially complementary in that the two regions can interact in a predictable fashion to produce the double-stranded or “stem” portion of the stem-loop motif. Stem-loop motifs are well known in the art. The actual primary sequence of nucleotides within the stem-loop structure is not critical to the practice of the invention, as long as the secondary structure is generally present. Those of ordinary skill in the art will be able to determine, given a nucleic acid having a primary sequence of nucleotides, whether the nucleic acid is able to form a stem-loop motif. Non-limiting examples of RNAs having stem-loop motifs can be seen in FIGS. 1A-1KK and FIGS. 3A-3BB. For example, in FIG. 1G, the “loop” portion of the stem-loop motif has four nucleotides, while the remainder of the molecule is the “stem” portion of the stem-loop motif. In FIG. 1F, the “loop” portion of the stem-loop motif has 13 nucleotides, while in FIG. 1J, the “loop” portion of the stem-loop motif itself contains 4 stem-loop motifs.

Certain stem-loop motifs of the present invention can be represented by a structure: 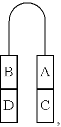
where 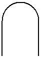
includes a “loop” motif as previously described (alone, or in combination with a “stem” portion). Examples of motifs that may be suitable include, but are not limited to, SEQ ID NO:17 to SEQ ID NO: 53, SEQ ID NO: 62 to SEQ ID NO: 89, and/or variants thereof. Other examples of stem-loop or hairpin motifs are given in the sequence listings above. “Variants,” as used herein in reference to nucleic acids, refers to sequences of nucleotides similar to a given nucleotide sequence, but with minor differences, for example, in reference to stem-loop motifs, such that the stem-loop motif generally retains its secondary stem-loop structure. For example, the variant sequence may be substantially the same as the reference sequence, with at least about 75%, at least about 80%, at least about 85%, at least about 90%, at least about 95%, at least about 96%, at least about 97%, at least about 98%, at least about 99%, at least about 99.5%, or 100% of the nucleotides being the same as the reference sequence. In other cases, the variant sequence may have a maximum of 40 mismatches (e.g., where one base of the variant sequence is not identical to the reference sequence, for example, due to additions, deletions, substitutions, bulges, etc.), 30 mismatches, 20 mismatches, 10 mismatches, or 7 mismatches, as compared to the reference sequence. In still other cases, the variant sequence may have a maximum of 0, 1, 2, 3, 4, 5, or 6 mismatches, as compared to the reference sequence.

Also, in the structure above, 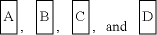
each represent nucleic acid sequences that are part of the stem-loop structure, i.e., 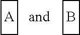
may be substantially complementary to each other, and/or 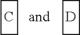
may be substantially complementary to each other. Thus, if the above structure represents a precursor miRNA, with 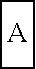
encoding the final, mature miRNA, the precursor miRNA, upon cleavage by the plant cell, may produce a final structure 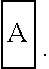
Typically, in a precursor miRNA, 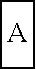
has between 20 to 24 nucleotides, inclusive, as previously described. on-limiting examples of 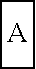
include SEQ ID NO: 1 to SEQ ID NO: 16 and SEQ ID NO: 54 to SEQ ID NO: 61. Still other examples are given in the sequence listings above. In such precursor miRNAs, 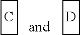
may each independently be absent or comprise at least one nucleotide. As one particular example, in the miRNA identified as MIR156a of Arabidopsis in FIG. 1A, 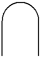
is SEQ ID NO: 17, 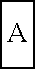
is SEQ ID NO: 1, 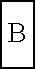
is substantially complementary to 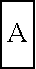
with 2 mismatches, 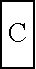
is C, and 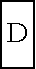
is G, which is perfectly complementary to. 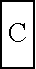
As another example, in the miRNA identified as MIR167a of Arabidopsis in FIG. 1EE, 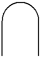
is SEQ ID NO:47, 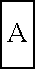
is SEQ ID NO: 12, 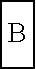
is substantially complementary to 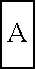
with 1 mismatch, and 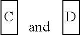
are both absent.

The precursor miRNA may include homologous or heterologous stem-loop and miRNA sequence components. A “homologous” sequence is an identifiable segment of nucleic acid within a larger nucleic acid molecule that is found in association with the larger molecule in nature. A “heterologous” structure is an identifiable segment of nucleic acid within a larger nucleic acid molecule that is not found in association with the larger molecule in nature. Transfection of a precursor miRNA containing a heterologous sequence into a cell may result in the formation of a transgenic plant cell. As used herein, the term “transgenic,” when used in reference to a plant (i.e., a “transgenic plant”) refers to a plant that contains at least one heterologous gene in one or more of its cells. Thus, in some instances, the precursor miRNA will include a stem-loop structure that is not ordinarily associated in nature with the miRNA with which it is associated in the precursor molecule. In a homologous structure the two components are ordinarily found in association with one another in nature. A heterologous precursor miRNA may be produced by replacing a portion (e.g., the homologous miRNA from the stem-loop structure) of a precursor miRNA taken from a plant cell with a sequence substantially complementary to another gene, for example, a gene that is desired to be inhibited or otherwise altered. The portion of the precursor miRNA that is substantially complementary to the replaced miRNA portion may also be replaced with a sequence that is substantially complementary to the gene newly added to the precursor miRNA. In some cases, a heterologous precursor miRNA may be produced by selecting a sequence substantially complementary to a gene that is desired to be inhibited or otherwise altered, pairing it with a substantially complementary, and adding the paired sequence to a stem-loop structure, which may be artificially generated in some cases. For example, with reference to the above structure, a precursor miRNA may be created by selecting a sequence substantially complementary to a gene that is desired to be inhibited or otherwise altered 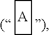
pairing it with a substantially complementary sequence 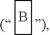
and adding a sequence that includes a stem-loop motif 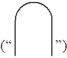
(other sequences may optionally be included within the stem-loop motif as well, in some embodiments). Optionally, one or more other sequences may also be added to the precursor miRNA

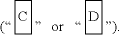

Plant cells may process the precursor miRNA into the mature miRNA through the action of certain nucleases. One non-limiting example is Dicer, an RNAse III enzyme. Dicer acts on many nucleic acids, in addition to precursor miRNA. For example, long double-stranded RNA can be processed by Dicer into many siRNAs, as previously discussed. Although these siRNAs are initially short double-stranded species with 5′-phosphates and 2-nucleotide 3′-overhangs characteristic of RNAse III cleavage products, they eventually can become incorporated as single-stranded RNAs into a ribonucleoprotein complex known as the RNA-induced silencing complex (“RISC”). The RISC identifies target messages based on antisense complementarity between the siRNA and the mRNA, and then a RISC endonuclease cleaves the mRNA near the middle of the siRNA complementarity region.

Like siRNAs, precursor miRNAs may also be processed by Dicer to produce mature miRNAs having a similar length as siRNAs, and possessing 5′-phosphate and 3′-hydroxyl termini. The miRNAs also may be incorporated into a ribonucleoprotein complex, known as the miRNP, which is similar to the RISC. miRNAs can also direct the cleavage of their mRNA targets as if they were functioning as siRNAs within the RISC complex.

Despite the chemical, biochemical, and mechanistic similarities to siRNAs, there are several differences between miRNAs and siRNAs, both in origin and evolutionary conservation, and in identification. Those of ordinary skill in the art, in applying one or more of these indications, will be able to determine whether a given RNA is an miRNA or an siRNA. (1) miRNAs derive from genomic loci distinct from other recognized genes, whereas siRNAs derive from mRNAs, transposons, viruses, or heterochromatic DNA. (2) miRNAs are processed from transcripts that can form local RNA hairpin precursor structures, whereas siRNAs are processed from long bimolecular RNA duplexes or extended hairpins. (3) A single miRNA molecule ultimately accumulates from one arm of each miRNA hairpin precursor molecule, whereas many different siRNAs accumulate from both strands of siRNA precursors. (4) miRNA sequences are nearly always conserved in related organisms, whereas siRNA sequences are rarely conserved in related organisms. (5) siRNAs mediate the silencing of the same (or very similar) genes from which they originate, whereas miRNAs are encoded by their own genes and regulate different genes.

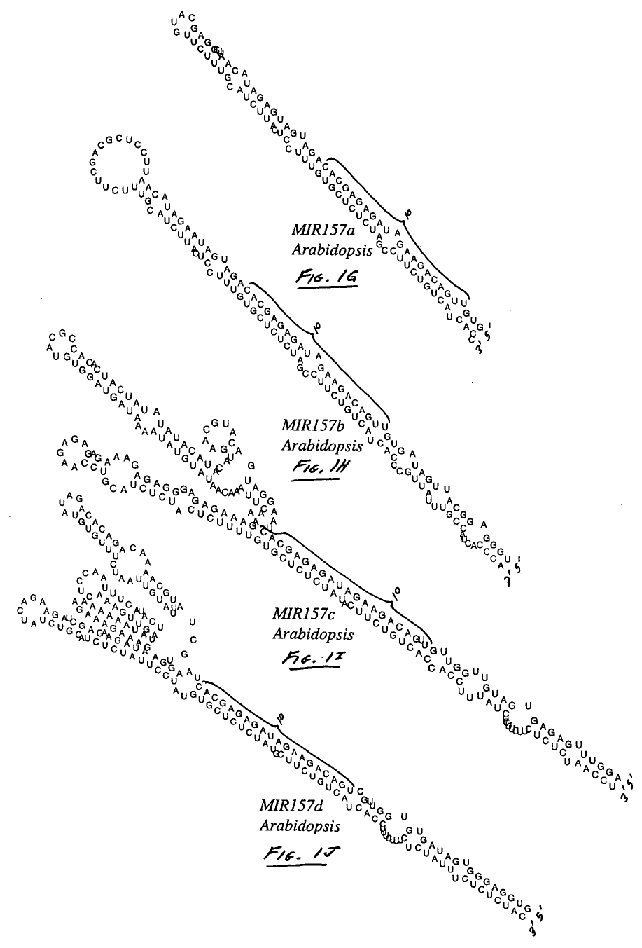

Various non-limiting examples of mature miRNAs include SEQ ID NO:1 through SEQ ID NO: 16, each derived from Arabidopsis thaliana (see also FIG. 2), and SEQ ID NO: 54 through SEQ ID NO: 61, each derived from Oryza sativa. miRNAs may be derived from other plant species as well, for example, other plant species including other Arabidopsis species, other Oryza species, or the like. Still other examples are given in the sequence listings above. The presence of miRNAs in plants expands the known phylogenetic distribution of this class of tiny noncoding RNAs, and suggests that miRNAs arose early in eukaryotic evolution, before the last common ancestor of plants and animals. The presence of miRNAs in plants also suggests that the developmental defects of carpel factory (caf), a mutation in a Dicer homolog, and mutations in the ARGONAUTE family proteins could result from miRNA processing defects. For instance, the accumulation of plant miRNAs may be substantially reduced in the caf mutant.

Precursor miRNA sequences are typically produced by transcribing a portion of the cell's DNA into RNA. Thus, another aspect of the invention relates to a nucleotide sequence able to be transcribed by a plant cell into precursor miRNA that is cleavable by the plant cell to produce miRNA. The gene to be partially or totally inhibited, or otherwise altered, may be any plant cell gene that is capable of being transcribed into a protein. Many examples of such genes are well known in the art and others have yet to be identified. The particular gene to be inhibited will depend on the desired change to the cell. The methods and compositions of the invention are not limited to a particular gene. The nucleotide sequence may be isolated, e.g., from plant cells, according to certain embodiments, and the nucleotide sequence may be either DNA or RNA. Those of ordinary skill in the art will be able to determine if a given nucleotide sequence encodes a precursor miRNA sequence. In some embodiments, as further discussed below, the nucleotide sequence may be delivered to a plant cell. The nucleotide sequence may then be expressed by the plant cell.

Precursor miRNAs, according to the invention, are not limited to wild-type or homologous precursor miRNAs. Certain aspects of the invention contemplates modified precursor miRNAs, where a portion of the precursor miRNA, such as the region encoding the mature miRNA, is replaced in some fashion with another miRNA sequence. Any suitable miRNA sequence may be used, for example, miRNA sequences directed to the inhibition of a gene, partially or totally, within the plant cell. In some cases, the new miRNA sequence added to the precursor miRNA may be shorter or longer than the original miRNA sequence. For instance, one aspect of the invention is generally directed to an isolated precursor miRNA able to inhibit a gene in a plant cell. A portion of a precursor miRNA, or a nucleotide sequence able to be transcribed by a plant cell into precursor miRNA, may be replaced with a sequence substantially complementary to a gene to be inhibited. Methods using such isolated precursor miRNA, or nucleotide sequences encoding such precursor miRNA, to partially or totally inhibit, or otherwise alter a gene are also provided in certain embodiments of the present invention. For instance, a precursor miRNA may be inserted into a plant cell, and/or a nucleotide sequence encoding a precursor miRNA may be inserted into a plant cell such that the nucleotide sequence can be transcribed by the plant cell into precursor miRNA.

Thus, the present invention also provides, according to various aspects, methods and compositions for the expression of precursor miRNA in plants, for example, to inhibit a gene. In some cases, the expression of miRNA and/or precursor miRNA in a plant cell may be altered by altering the environment that the cell is in. For example, a concentration of a species, such as SO₄²⁻, may be altered, which, in some cases, causes the plant cell to alter its expression of miRNA, i.e., by stimulating or inhibiting expression of miRNA.

Any method or delivery system may be used for the delivery and/or transfection of the precursor miRNA, or a nucleotide sequence able to be transcribed to produce precursor miRNA in the cell. The precursor miRNA, or the nucleotide sequence able to be transcribed to produce precursor miRNA, may be delivered to the plant cell alone, or in combination with other agents. Examples of delivery systems include, but are not limited to, particle gun technology, colloidal dispersion systems, electroporation, vectors, and the like. In its broadest sense, a “delivery system,” as used herein, is any vehicle capable of facilitating delivery of a nucleic acid (or nucleic acid complex) to a cell and/or uptake of the nucleic acid by the cell. Other example delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell).

The term “transfection,” as used herein, refers to the introduction of a nucleic acid into a cell, for example, a precursor miRNA, or a nucleotide sequence able to be transcribed to produce precursor miRNA. Transfection may be accomplished by a wide variety of means, as is known to those of ordinary skill in the art. Such methods include, but are not limited to, Agrobacterium-mediated transformation (e.g., Komari, et al., Curr. Opin. Plant Biol., 1:161 (1998)), particle bombardment mediated transformation (e.g., Finer, et al., Curr. Top. Microbiol. Immunol., 240:59 (1999)), protoplast electroporation (e.g., Bates, Methods Mol. Biol., 111:359 (1999)), viral infection (e.g., Porta and Lomonossoff, Mol. Biotechnol. 5:209 (1996)), microinjection, and liposome injection. Standard molecular biology techniques are common in the art (e.g., Sambrook, et al., Molecular Cloning: A Laboratory Manual, 2^{nd} ed., Cold Spring Harbor Laboratory Press, New York (1989)). For example, in one embodiment of the present invention, Arabidopsis or another plant is transformed with a gene encoding a precursor miRNA using Agrobacterium.

In one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry RNA or other nucleic acids into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of a nucleic acid (or nucleic acid complex) into the cell, for example, precursor miRNA, or a nucleotide sequence able to be transcribed to produce precursor miRNA. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which-range in size from 0.2 to 4.0 microns, can encapsulate large macromolecules within the aqueous interior and these macromolecules can be delivered to cells in a biologically active form (e.g., Fraley, et al., Trends Biochem. Sci., 6:77 (1981)).

Lipid formulations for the transfection and/or intracellular delivery of nucleic acids are commercially available, for instance, from QIAGEN, for example as EFFECTENE® (a non-liposomal lipid with a special DNA condensing enhancer) and SUPER-FECT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (“DOTMA”) and dimethyl dioctadecylammonium bromide (“DDAB”). Liposomes are well known in the art and have been widely described in the literature, for example, in Gregoriadis, G., Trends in Biotechnology 3:235-241 (1985).

Electroporation may be used, in another set of embodiments, to deliver a nucleic acid (or nucleic acid complex) to the cell, e.g., precursor miRNA, or a nucleotide sequence able to be transcribed to produce precursor miRNA. “Electroporation,” as used herein, is the application of electricity to a cell in such a way as to cause delivery of a nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by those of ordinary skill in the art.

In yet another set of embodiments, a nucleic acid (e.g., precursor miRNA, or a nucleotide sequence able to be transcribed to produce precursor miRNA) may be delivered to the cell in a vector. In its broadest sense, a “vector” is any vehicle capable of facilitating the transfer of the nucleic acid to the cell such that the nucleic acid can be processed and/or expressed in the cell. The vector may transport the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors, including those cells previously described.

In general, vectors useful in the invention include, but are not limited to, plasmids, phagemids, viruses, other vehicles derived from viral or bacterial sources that have been manipulated by the insertion or incorporation of the nucleotide sequences (or precursor nucleotide sequences) of the invention. Viral vectors useful in certain embodiments include, but are not limited to, nucleic acid sequences from the following viruses: retroviruses; adenovirus, or other adeno-associated viruses; mosaic viruses such as tobamoviruses; potyviruses, nepoviruses, and RNA viruses such as retroviruses. One can readily employ other vectors not named but known to the art. Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) are well known to those of ordinary skill in the art. Examples of standard protocols can be found in Kriegler, M., Gene Transfer and Expression, A Laboratory Manual, W.H. Freeman Co., New York (1990), or Murry, E. J. Ed., Methods in Molecular Biology, Vol. 7, Humana Press, Inc., Cliffton, N.J. (1991).

Another-example of a virus for certain applications is the adeno-associated virus, which is a double-stranded DNA virus. The adeno-associated virus can be engineered to be replication-deficient and is capable of infecting a wide range of-cell types and species. The adeno-associated virus further has advantages, such as heat and lipid solvent stability; high transduction frequencies in cells of diverse lineages; and/or lack of superinfection inhibition, which may allow multiple series of transductions.

Another vector suitable for use with the invention is a plasmid vector. Plasmid vectors, have been extensively described in the art and are well-known to those of skill in the art. See, e.g., Sambrook, et al., Molecular Cloning: A Laboratory Manual, Second Edition, Cold Spring Harbor Laboratory Press, 1989. These plasmids may have a promoter compatible with the host cell, and the plasmids can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence (which can, for instance, then be cleaved or otherwise processed within the cell to produce a precursor miRNA). These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell (e.g., to produce a precursor miRNA). The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). In some embodiments, the nucleic acid is linked to a gene expression sequence which permits expression of the nucleic acid in a plant cell. A sequence which permits expression of the nucleic acid in a plant cell is one which is selectively active in the particular plant cell and thereby causes the expression of the nucleic acid in these cells. Those of ordinary skill in the art will be able to easily identify promoters that are capable of expressing a nucleic acid in a cell based on the type of plant cell.

The selection of a particular promoter and enhancer depends on what cell type is to be used and the mode of delivery. For example, a wide variety of promoters have been isolated from plants and animals, which are functional not only in the cellular source of the promoter, but also in numerous other plant species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus (“CaMV”). Promoters that have been isolated and reported for use in plants include ribulose-1,3-biphosphate carboxylase small subunit promoter, phaseolin promoter, etc.

Exemplary viral promoters which function constitutively in eukaryotic cells include constitutive promoters from plants are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those-of ordinary skill in the art.

Thus, a variety of promoters and regulatory elements may be used in the expression vectors of the present invention. For example, in some preferred embodiments an inducible promoter is used to allow control of nucleic acid expression through the presentation of external stimuli (e.g., environmentally inducible promoters). Thus, the timing and amount of nucleic acid expression can be controlled in some cases. Non-limiting examples of expression systems, promoters, inducible promoters, environmentally inducible promoters, and enhancers are well known to those of ordinary skill in the art. Examples include those described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472. A general descriptions of plant expression vectors and reporter genes can also be found in Gruber, et al., “Vectors for Plant Transformation,” in Methods in Plant Molecular Biology & Biotechnology, Glich, et al., Eds., p. 89-119, CRC Press (1993).

An efficient plant promoter that may be used is an “overproducing plant promoter.” Overproducing plant promoters that may be used in this invention include the promoter of the small sub-unit (“ss”) of the ribulose-1,5-biphosphate carboxylase from soybean (e.g., Berry-Lowe, et al., J. Molecular & App. Genet., 1:483 (1982)), and the promoter of the chorophyll a-b binding protein. These two promoters are known to be light-induced in eukaryotic plant cells. For example, see Cashmore, Genetic Engineering of plants: An Agricultural Perspective, p. 29-38; Coruzzi, et al., J. Biol. Chem., 258:1399 (1983); and Dunsmuir, et al., J. Molecular & App. Genet., 2:285 (1983).

As used herein, an “expression element” can be any regulatory nucleotide sequence, such as a promoter sequence or promoter-enhancer combination, which facilitates the efficient expression of a nucleic acid, for example, precursor miRNA, or a nucleotide sequence able to be transcribed to produce precursor miRNA. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (“HPTR”), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus, Rous sarcoma virus, cytomegalovirus, the long terminal repeats of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

Using any gene transfer technique, such as the above-listed techniques, an expression vector harboring the nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (“GFP”) is used to allow simple selection of transfected cells and to monitor expression levels. Non-limiting examples of such vectors include Clontech's “Living Colors Vectors” pEYFP and pEYFP-C1.

In some cases, a selectable marker may be included with the nucleic acid being delivered to the cell. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

Optionally, germ line cells may be used in the methods described herein rather than, or in addition to, somatic cells. The term “germ line cells” refers to cells in the plant organism which can trace their eventual cell lineage to either the male or female reproductive cell of the plant. Other cells, referred to as “somatic cells” are cells which give rise to leaves, roots and vascular elements which, although important to the plant, do not directly give rise to gamete cells. Somatic cells, however, also may be used. With regard to callus and suspension cells which have somatic embryogenesis, many or most of the cells in the culture have the potential capacity to give rise to an adult plant. If the plant originates from single cells or a small number of cells from the embryogenic callus or suspension culture, the cells in the callus and suspension can therefore be referred to as germ cells. In the case of immature embryos which are prepared for treatment by the methods described herein, certain cells in the apical meristem region of the plant have been shown to produce a cell lineage which eventually gives rise to the female and male reproductive organs. With many or most species, the apical meristem is generally regarded as giving rise to the lineage that eventually will give rise to the gamete cells. An example of a non-gamete cell in an embryo would be the first leaf primordia in corn which is destined to give rise only to the first leaf and none of the reproductive structures.

In one aspect, the present invention provides any of the above-mentioned compositions in kits, optionally including instructions for use of the composition e.g., for the inhibition of a gene. The “kit” typically defines a package including one or more compositions of the invention and the instructions, and/or analogs, derivatives, or functionally equivalent compositions thereof. Thus, for example, the kit can include a description of use of the composition for participation in any technique associated in the inhibition of genes. The kit can include a description of use of the compositions as discussed herein. Instructions also may be provided for use of the composition in any suitable technique as previously described. The instructions may be of any form provided in connection with the composition.

The kits described herein may also contain one or more containers, which may contain the inventive composition and other ingredients as previously described. The kits also may contain instructions for mixing, diluting, and/or administrating the compositions in some cases. The kits also can include other containers with one or more solvents, surfactants, preservative and/or diluents (e.g., normal saline (0.9% NaCl), or ⁵⁰/o dextrose) as well as containers for mixing, diluting and/or administrating the compositions.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the active compound(s) within the composition. Suitable solvents are well known, for example as previously described, and are available in the literature.

The invention also involves, in another aspect, promotion of the inhibition of genes according to any of the systems or methods described herein. As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention. “Instructions” can define a component of promotion, and typically involve written instructions on or associated with packaging of compositions of the invention. Instructions also can include any oral or electronic instructions provided in any manner.

The present invention is further illustrated by the following examples, which in no way should be construed as further limiting.

### EXAMPLE 1

In this example, endogenous RNAs were cloned from Arabidopsis to illustrate the isolation of 16 plant miRNAs. A brief description of plant growth and RNA isolation is as follows. Total RNA from wild-type Arabidopsis thaliana (Columbia accession) was isolated from 6-day-old seedlings grown on agar-based medium overlaid with filter paper, and from flowers and stems of 4-week-old plants grown in soil using Trizol (GIBCO BRL). Total RNA was prepared from leaves and siliques using a modification of the method described in Nagy, et al., “Analysis of Gene Expression in Transgenic Plants,” in Plant Molecular Biology Manual, Part B4, Gelvin, et al., Eds., p.1-29, Kluwer, Dordrect (1988), in which LiCl precipitation was replaced by ethanol precipitation. For isolation of RNA from carpel factory plants, progeny of CAF/caf heterozygous plants (in the Landsberg erecta accession) were grown on medium supplemented with 12 microgram/ml kanamycin for 8 days, after which kanamycin-resistant individuals were transferred to soil and grown for an additional 24 days under continuous illumination. Plants were then scored as having (caf/caf) or lacking (CAF/caf) the carpel factory phenotype (Jacobsen, et al., Development, 126: 5231 (1999)), and RNA was prepared from leaves, stems, and flowers using a modification of the Nagy method. Wild-type plants (Landsberg erecta accession) were processed similarly, except that seeds were originally sown on medium lacking kanamycin.

For RNA analysis, endogenous 18-nucleotide to 26-nucleotide RNAs from seedlings and flowers were isolated from total RNA by 15% PAGE and cloned as described in Lau, et al., Science, 294:858 (2001). For Northern analysis, 20 microgram of total RNA per lane was separated on a 15% polyacrylamide gel, electroblotted to a nylon membrane, and hybridized to end-labeled anti-sense DNA probes, using procedures similar to those described in Lee, et al., Science, 294:862 (2001).

Sequences of RNA clones were compared with the Arabidopsis genome downloaded from the National Center for Biotechnology Information at ftp://ncbi.nlm.nih.gov/genbank/genomes/A_thaliana/on Aug. 13, 2001. Predicted secondary structures were generated using the Zucker folding algorithm and manually inspected for fold-backs with the RNA sequence in the stem, as is characteristic of metazoan miRNAs. To identify Oryza sativa homologs, the miRNAs were compared with the rice genome sequence downloaded from the Beijing Genomics Institute Web site at http://btn.genomics.org.cn/rice (first draft) using the BLAST algorithm, and the adjoining sequences were analyzed for fold-back secondary structures as described above.

In this example, by using methods designed to clone Dicer cleavage products, which are 20-nt to 24-nt RNAs with 5′-phosphate and 3′-hydroxyl groups, approximately 200 tiny RNAs were cloned from Arabidopsis seedlings and approximately 100 were cloned from flowers. Of these, 18 sequences were represented by more than one clone and were the subject of further analysis, as described here. Of these 18 RNAs, 16 had striking similarities to the miRNAs of animals and have therefore been named miR156 through miR171, with genes designated MIR156 through MIR171 (FIG. 2). Six of the miRNAs represent three pairs of closely related RNA sequences differing only by one or two nucleotides. Interestingly, most of the plant miRNAs begin with a U, a trend previously observed in animals.

Five of the plant miRNA sequences found in this example have a single copy in the Arabidopsis genome, whereas each of the other 11 sequences correspond to multiple (2-7) loci (FIG. 2), possibly because of duplications in the Arabidopsis genome. As expected for miRNA loci, nearly all (37 of 40) of the genomic loci lie outside of annotated segments of the genome, and thus do not correspond to previously identified genes. The three exceptions are for a single miRNA, miR171. Furthermore, each of these 37 loci placed the cloned RNA sequence in a context where it can pair with a nearby genomic segment to form a dsRNA hairpin structure resembling those thought to be required for Dicer processing of miRNAs (FIG. 4). The mature miRNA can be processed from either the 5′ or the 3′ arm of the fold-back precursor. Each miRNA with multiple matches to the genome was found to be present on the same arm of its potential precursors, suggesting that these loci share a common ancestry.

Fold-back secondary structures of Arabidopsis miRNA precursors MIR156a-f, MIR169, and MIR170, shown in FIG. 4, were determined by the RNAfold program. The miRNA portion 10 of each miRNA precursor is as indicated. For miR156 and miR169, RNAs from the other side of the fold-back 20 were each cloned once. The duplexes that could form between these substantially complementary RNAs and the miRNA portions 10 from the other strand have about 2-nucleotide 3′ overhangs, which is a characteristic of Dicer cleavage.

The sizes of the Arabidopsis hairpins were more variable than those of animals. For example, Caenorhabditis elegans miRNAs tend to be cleaved from precursors approximately 70 nucleotides in length, with the mature miRNA located only 2 to 10 base pairs from the terminal loop of the stem-loop. Although some of the Arabidopsis precursors resemble those of C. elegans, others are larger, as seen for the approximately 190 nucleotides precursor of miR169.

For most (14 out of 16) of the plant miRNAs, sequences were cloned from only one arm of the fold-back precursor. For two loci, a single 21 nucleotide sequence was also cloned from the other arm of the fold-back (FIG. 4). The disparity in cloning frequency between the two sides, 16:1 in the case of MIR156, was similar to that seen for metazoan miRNAs. The isolation of these two sequences generated from the opposite arm of the predicted fold-back supports the existence of these stem-loops as miRNA precursors. Furthermore, the duplexes that could be formed between the sequences isolated from both sides of the stems have 2-nucleotide 3′ overhangs (FIG. 4), suggesting that they are products of a Dicer-like activity similar to that which processes the metazoan miRNAs.

Northern analysis confirmed that the 16 miRNAs were stably expressed as approximately 21-nucleotide RNAs (FIG. 5). All of the 16 miRNAs were expressed at some level in seedlings, leaves, stems, flowers, and siliques (seed pods). Whereas miR163 accumulated in all tissues, with only slightly lower levels in seedlings and siliques, other miRNAs had quite variable levels among the tissues tested. For example, miR157 was most highly expressed in seedlings, and miR171 was most highly expressed in flowers, suggesting that they play roles in the development of these stages/organs. The size of the RNAs detected approximately matched those that were cloned. In some cases, RNAs of two sizes were detected, reflecting the heterogeneity of the cloned sequences (FIG. 3). For example, a probe to miR156 detected both 20 and 21nucleotide RNAs, and the miR156 cloned were of both sizes. As another example, miR167, a 21 -nucleotide RNA accumulated in all tissues except stem tissue, where a 22-nucleotide RNA accumulated instead. This may reflect either differential transcription of the two MIR167 genes that have differently processed precursors, and/or tissue-specific differences in the Arabidopsis miRNA processing machinery.

FIG. 5 illustrates developmental expression of Arabidopsis miRNAs. Total RNA from Columbia seedlings (Se), leaves (L), stems (St), flowers (F), and siliques (Si) was analyzed on Northern blots by hybridization to end-labeled DNA oligonucleotide probes-complementary to the miRNA. The lengths of end-labeled RNA oligonucleotides run as a size marker (M) are noted to the left of each panel. Although miR165 and miR166 sequences and miR170 and miR171 sequences were too closely related to be reliably distinguished by hybridization probes, miR156 and miR157 can be specifically recognized, as reflected in their different levels of expression in seedlings and siliques. miR159 and miR164 showed a similar expression profile to miR165, whereas miR160, miR162, and miR168 had similar profiles to miR158. The low expression level of most miRNAs in leaves and siliques may reflect a difference in the efficiency of small RNA recovery with the RNA isolation method used for these two tissues, as previously discussed. Blots were stripped and reprobed with an oligonucleotide probe complementary to U6 as a loading control.

Although the presence of precursors in Arabidopsis was not detected on Northern blots, the potential for their production prompted this investigation as to whether the approximately 21 -nucleotide miRNAs was processed from a longer dsRNA by proteins homologous to those that generate metazoan miRNAs. Dicer is thought to cleave the double-stranded region of the miRNA precursors in Drosophila, C. elegans, and humans. Mutations have been isolated in only one of the four Dicer homologs in Arabidopsis, CARPEL FACTORY (CAF; also named SHORT INTEGUMENT or SIN1). The pleiotropic phenotypes associated with loss of CAF/SIN1 function, such as floral meristem proliferation defects, floral organ morphogenesis defects, and altered ovule development, emphasize the critical developmental role of RNAs processed by CAF.

Northern analysis showed that the expression level of the three miRNAs tested was significantly reduced in carpel factory homozygotes (FIG. 6). In this figure, expression of miR169 was shown to be dependent on CARPEL FACTORY. Total RNA from wild-type Landsberg erecta (CAF/CAF), heterozygous (CAF/caf), and homozygous (caf/caf) carpel factory leaves (L), stems (St), and flowers (F) was analyzed on a Northern blot. RNA size markers (M) are noted to the left. The blot probed for miR1 58 was stripped and reprobed with a U6 end-labeled DNA probe as a loading control.

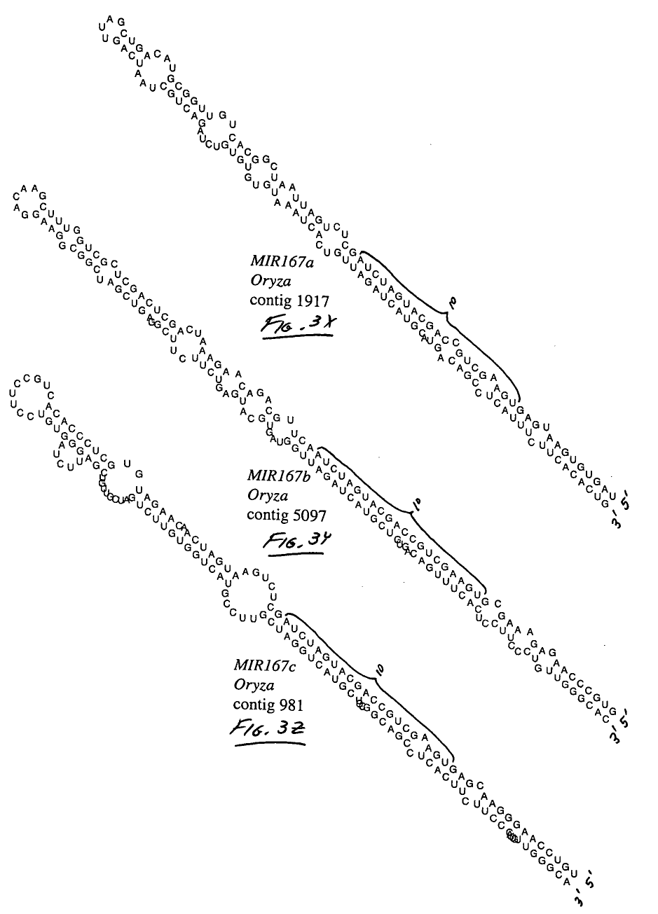

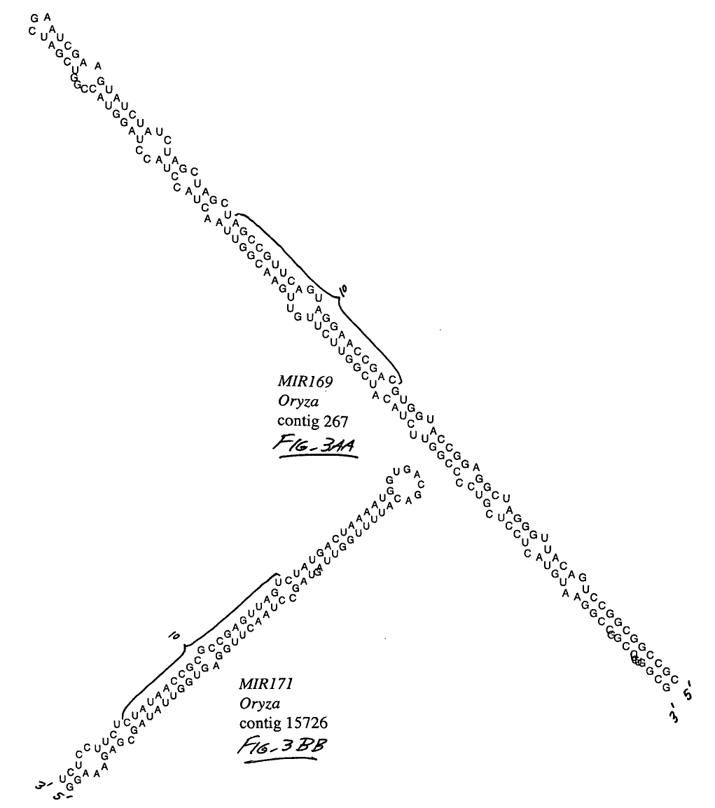

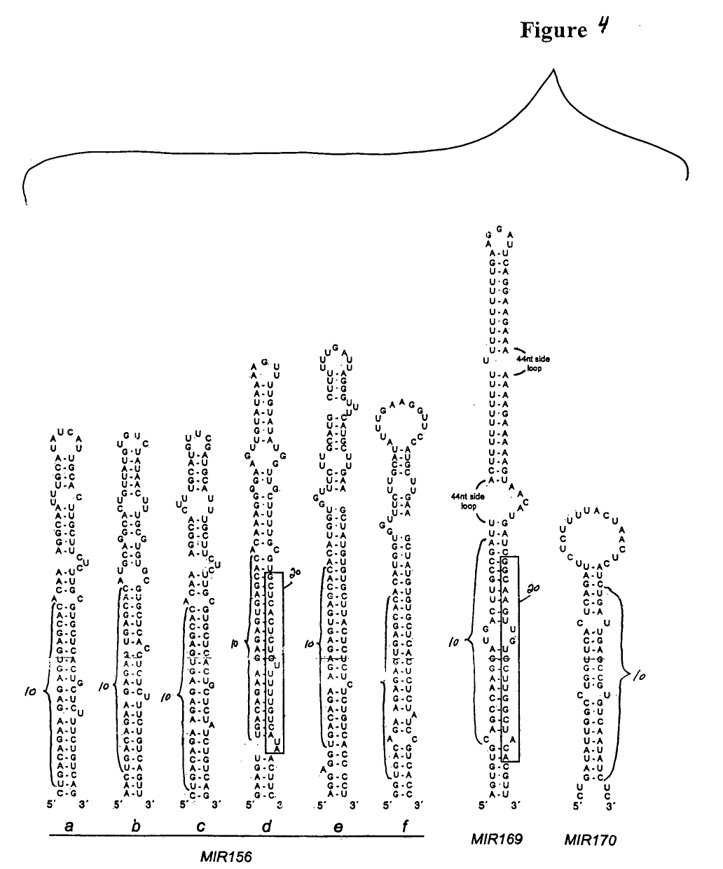

Similar miRNA sequences were found in different plant species. The evolutionary conservation of the miRNA sequences in different species indicated that they may have important biological functions. Eight Arabidopsis miRNAs had sets of identical matches in the genome of the rice Oryza sativa L. ssp. indica (FIGS. 7A-7B), which was estimated to have 92% functional coverage at the time of this analysis. With rare exceptions (noted in FIG. 3), these sets of Oryza homologs have adjacent sequences that could form stem-loop precursors analogous to those of Arabidopsis, with the miRNA sequence generally on the same arm of the precursor in both species. The Arabidopsis and Oryza sequences have drifted considerably in regions outside the miRNA sequence, but selective pressure can be seen in the segments predicted to base-pair with the miRNAs, resulting in only a few base changes in these segments and a conserved overall propensity for dsRNA formation (FIGS. 7A-7B). For each set of related loci, the precursor duplexes extended beyond the length of the miRNA, but the sequence of the flanking duplex RNA was variable. This conservation in secondary structure accompanied by variability in sequence provides added evidence that the secondary structural context of these RNAs is important, presumably for their processing from stem-loop precursors. FIG. 7A illustrates miR162 homologs, while FIG. 7B illustrates miR164 homologs. Sequence homology is seen within the miRNA sequence 10 within the precursor miRNA, its paired sequences, and a few base pairs adjacent to the miRNA. The remainder of the sequence has drifted considerably, with the main constraint being the formation of the stem-loop structures.

In nematodes, lin-4 and let-7 RNA recognize their target mRNAs through limited base-pairing to complementary sites within the 3′ UTR of their targets. The largest regions of uninterrupted complementarity were only about 8 nucleotides. Similarly, the plant miRNA sequences did not perfectly match coding regions, with the exception of miR171, which was found to have four matches to the genome. One locus is 0.5 kb from the nearest predicted coding region and adjacent to genomic sequence that can form a classical miRNA precursor, consistent with the idea that it is thus miRNA. In support, it was observed that a closely related sequence, miR170, was also cloned multiple times and has all the characteristics of the other plant miRNAs. However, the other three MIR171 loci differ from those of the other miRNAs (FIG. 3). These loci were anti-sense to the coding region of three SCARECROW-like genes of the GRAS family of putative transcription factors. This is the first example of a convincing miRNA candidate that is also the perfect anti-sense match to a coding region. Although this miR171 sequence identity may be a coincidence, the targets of this 2 1 -nucleotide RNA could include these three SCARECROW-like genes. miR171 (and perhaps the related miRNA, miR170) may act like a translational regulator similar to the lin-4 and let- 7 RNAs, or it may pair with these three genes for a very different type of regulatory interaction. miR171 could direct cleavage of the messages as if it were an siRNA of the RNAi pathway, or it could direct a nucleic acid modification such as the methylation of genomic DNA seen in PTGS and transcriptional gene silencing of plants. The five perfect matches to miR171 in Oryza also included one miRNA homolog and four anti-sense matches to SCARECROW family members. This observation indicates that these SCARECROW segments may be conserved based on their function as miRNA targets, in addition to their function in coding proteins.

It is believed that the other two RNAs cloned multiple times, SEQ ID NO: 92 (“Sequence C” in FIG. 8) and SEQ ID NO: 95 (“Sequence F” in FIG. 8), are not likely to be miRNAs. The arrows 30 in FIG. 8 represent the two predicted genes in this region (At2g39670 and At2g39680), and vertical lines 35 (labeled A-F) represent the genomic positions of the six cloned RNAs (SEQ ID NO: 90-SEQ ID: 95, labeled A-F). Sequences of the RNAs are listed, with cloning frequencies in parentheses. Expression of SEQ ID NO: 95, but not SEQ ID NO: 92, was detected on Northern blots. Nonetheless, neither appeared to have the potential to form extended pairing with the adjoining sequence like that seen for the other 16 sequences. Both of these sequences matched single loci in the same 2.3-kb region of Chromosome 2 that is also the source of four other approximately 22-nucleotide RNAs that were cloned once (FIG. 8, SEQ ID NO: 90, SEQ ID NO: 91, SEQ ID NO: 93, and SEQ ID NO: 94). It is believed that these RNAs are unlikely to be simply degradation products of mRNAs. Only two of these six sequences corresponded to the same DNA strand as the two predicted protein-coding genes in this 2.3-kb region. Moreover, one of the single-clone RNAs (FIG. 8, SEQ ID NO: 91, “Sequence B”) was a 2-nucleotide-offset reverse-complement of SEQ ID NO: 92. A duplex formed between them would have 1-nucleotide and 2-nucleotide 3′ overhangs, reminiscent of Dicer cleavage products during RNAi. The high density of 21-nucleotide to 22-nucleotide RNAs cloned from this region may implicate either endogenous RNAi or some other, unknown Dicer-mediated event.

### EXAMPLE 2

In this example, miRNAs were demonstrated that have near-perfect complementarity to mRNAs, particularly transcription factor mRNAs. Additionally, regulatory targets for 14 of the 16 miRNAs were identified and studied by searching for mRNAs capable of base pairing with three or fewer mismatches to one of the miRNAs. Many of these potential targets are members of gene families with roles in plant development. Particularly noteworthy targets include the PHABULOSA and PHAVULOTA mRNAs, for which the identification of miRNA complementary sites may explain the ectopic expression previously described for mutations in these genes.

The set of annotated Arabidopsis mRNA sequences was extracted from GenBank files, January 2002 release (Arabidopsis Genome Initiative, 2000). This set was searched for complementary sites to any of 16 miRNAs (described in Example 1; GenBank accession numbers AJ493620-AJ493656) using PatScan. When the miRNA was cloned as both a 20 and 21 nucleotide mRNA, the 21 nucleotide RNA was used (Example 1). Thus, the miRNA158 sequence was 20 nucleotides, the miR163 sequence was 24 nucleotides, and the remaining 14 miRNA sequences were 21 nucleotides. One mismatch was added to all miRNA158 complementary sites to compensate for their smaller size and the correspondingly greater chance of fortuitous complementarity. Complementary sites were also identified for 10 cohorts of 16 randomly permuted sequences that had identical sizes and base compositions to the authentic miRNAs. One mismatch was added to the sites complementary to the randomly permuted versions of miRNA158. Analogous searches for animal miRNA complementary sites queried annotated mRNAs in the D. melanogaster genome (GenBank October release) and annotated coding regions in the C. elegans genome (GenBank April 1999 release).

For each Arabidopsis target mRNA, the mRNAs of up to ten homologous Oryza proteins were predicted from the un-annotated Oryza contigs (a map of contiguous genomic DNA, assembled using overlapping cloned segments) by GenomeScan, a program that identifies genes within genomic sequence using homology to input protein sequences combined with an ab initio gene-finding algorithm. Complementary sites in this data set were identified by PatScan searches and homology to the Arabidopsis targets was confirmed by alignment of the inferred protein sequences (ClustalIX). One additional target homolog (TC79868) was found by searching the TIGR Rice Gene Index (9.0). For the control study, the identical GenomeScan/PatScan procedure was applied to the 44 Arabidopsis mRNAs with sites complementary (allowing up to 3 mismatches) to the 160 sequences in the 10 cohorts of randomized miRNAs.

To identify potential regulatory targets, Arabidopsis mRNAs were searched for sequences that were complementary, with four or fewer mismatches, to at least one of the 16 Arabidopsis miRNAs identified in Example 1. Gaps were not allowed, and G:U and other noncanonical pairs were treated as mismatches. To evaluate the significance of these hits to annotated mRNAs, parallel analyses were preformed using cohorts of randomly permuted sequences that had identical sizes and base compositions as the set of authentic miRNAs. There were substantially more antisense hits to the authentic miRNAs than to the randomized sequences (FIG. 9). This difference was especially striking at higher stringency; when summing the hits with two or fewer mismatches, the number of hits to the authentic miRNA set outnumbered those to the randomized cohorts by a ratio of 30:0.2 (FIG. 9). Considering the low probability of so many antisense hits occurring by chance, these results therefore indicate that these complementary sites reflect a functional relationship between the miRNAs and the identified mRNAs, i.e., that these protein-coding genes are regulatory targets of the miRNAs to which they are able to base-pair. In FIG. 9, sites complementary to the 16 Arabidopsis miRNAs with 0 to 4 mismatches are shown as solid bars. Identical searches with cohorts of 16 randomized RNAs are also illustrated (open bars, mean values from ten cohorts; error bars, one standard deviation). Two hits by similar miRNAs to the same complementary site within an mRNA were counted as separate hits (FIG. 10).

At lower stringencies, there were also significantly more hits with the authentic set of miRNAs than with the randomized cohorts. Most of the 31 hits with three mismatches were viable miRNA target candidates, although a few are likely to be mRNAs with fortuitous complementarity, as judged by the observation that on average the randomized cohorts hit 4.2 mRNAs when three mismatches were permitted (FIG. 9). Some hits with four mismatches may also be genuine targets. However, they were not included in the present analysis because of the greater likelihood that their complementarity is fortuitous, or occurs because they are targets of unidentified miRNAs related to the query set of 16 miRNAs from Example 1.

In FIG. 10, for each gene, the number of mismatches between the miRNA and the mRNA is indicated in parentheses. The sequences of three pairs of miRNAs (miR156/miR157, miR165/miR166, and miR170/miR171) were closely related and therefore were sometimes complementary to the same sites within the target mRNAs. Sites complementary to miR158 had an additional mismatch added to compensate for the fact that miR158 was at least 1 nucleotide shorter than the other miRNAs.

Potential regulatory targets with three or fewer mismatches were found for 14 of the 16 miRNAs (FIG. 10). Targets for the other two miRNAs could be identified through slight changes in the search algorithm. For example, miR163, one of the two miRNAs without predicted targets in Table 1 has extensive complementarity to members of the AtPP-like gene family (At1g66690, At1g66700, At1g66720, At3g44860, At3g44870). The 24 nucleotides of this miRNA paired to complementary sites within these mRNAs when a single-nucleotide gap was permitted near the 3′ terminus of the miRNA. Nonetheless, when searching for miRNA targets, permitting gaps did not substantially increase the number of targets predicted for the other miRNAs (data not shown). Perhaps a bulge is accommodated near the miRNA terminus more readily for miR163 because this miRNA is 24 nucleotides in length, which is 3 nucleotides longer than the other miRNAs queried.

In the cases where an miRNA was complementary to more than one mRNA, most of the potential targets were members of the same gene family (FIG. 10). The fraction of the gene family members with miRNA complementary sites varied considerably. Of the 16 Squamosa-promoter Binding Protein (“SBP”)-like genes inArabidopsis, ten had miR156 complementary sites. In contrast, the MYB and NAC families each have over 100 members in Arabidopsis, of which five in each case have sites complementary to miR159 or miR164, respectively. Unrelated miRNAs could also be complementary to different members of the same gene family, as illustrated by miR160 and miR167, which apparently target different members of the Auxin Response Factor family.

FIG. 11 illustrates the sequence context of certain miRNA complementary sites. FIG. 11A illustrates four miR165 complementary sites. These complementary sites lie within the START domain present in a subfamily of HDZip transcription factors. The altered protein sequences of the reported phv and phb gain-of-function alleles are as indicated. Each of these lesions also disrupted the miR165 complementary site. Amino acids conserved in a majority of the proteins are shaded. FIG. 11B illustrates miR156 complementary sites. All ten predicted targets contained the Squamosa-promoter Binding Protein (SBP) box, but the complementary sites were downstream of this conserved domain, within a poorly conserved protein-coding context or the 3′ UTR. Amino acids conserved in a majority of the proteins are shaded.

When considering the significance of multiple hits to the same gene family, it is important to address the possibility that these hits are merely the consequence of complementarity to a nucleotide sequence that encodes a critical protein motif. Indeed, for miR161, miR165, miR170, and miR171, the miRNA complementary sites were within the context of a domain strongly conserved among family members, as shown for the miR165 complementary sites (FIG. 11A). Therefore, the possibility that only a subset of the hits for these miRNAs are authentic targets could not be ruled out. This possibility was less likely in the cases of miR156, miR157, miR159, miR160, miR164, and miR169. The complementary sites for these miRNAs fell outside the conserved domains that define the families and instead fell within sequence contexts that were only weakly conserved among the family members, as shown for the miR156 sites within SBP-like mRNAs (FIG. 11B). Indeed, there are examples where the conservation of the miRNA complementary sites among family members must be independent of conserved protein function. In the case of the MYB genes with miR159 complementary sites, four genes translated the complementary site in the same reading frame, while the fifth gene translates the site in a different reading frame. In four other cases (miR156/157 to At1g53160, miR156 to At2g33810, and miR169 to At1g17590 and At1g54160), the miRNA complementary sites were not in the coding regions, but rather in the 3′ UTRs, as illustrated for miR156 and its complementary sites (FIG. 11B).

Many complementary sites observed in Arabidopsis are conserved in rice (Oryza sativa). Analysis of rice homologs focused on the seven miRNAs perfectly conserved in Oryza, for which complementary sites had been identified in Arabidopsis (FIG. 10). When using a three-mismatch cutoff, six of the seven conserved miRNAs (miR156, miR160, miR164, miR167, miR169, and miR171) had at least one potential target gene in Oryza homologous to a corresponding Arabidopsis target. As a control, an analogous study was performed using Arabidopsis hits to the cohorts of randomized miRNAs; no miRNA complementary sites were found in rice homologs of these Arabidopsis hits, even when four mismatches were allowed.

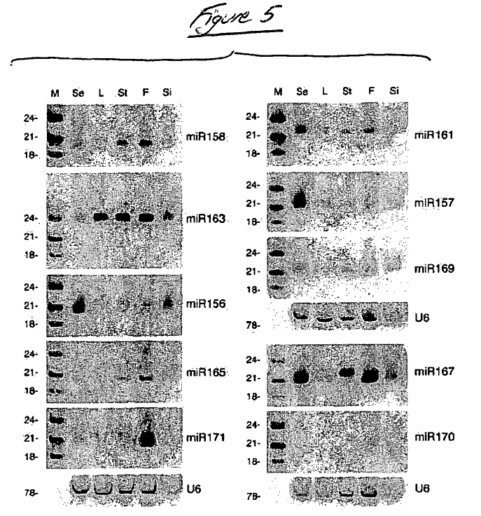

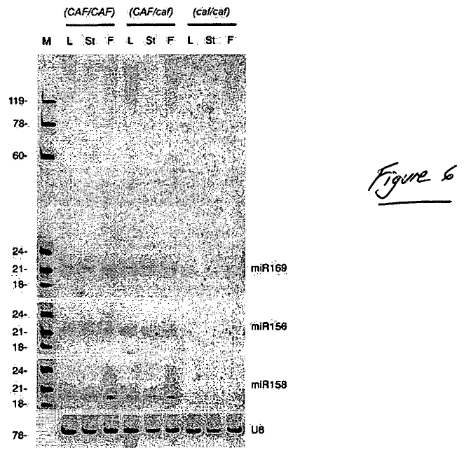

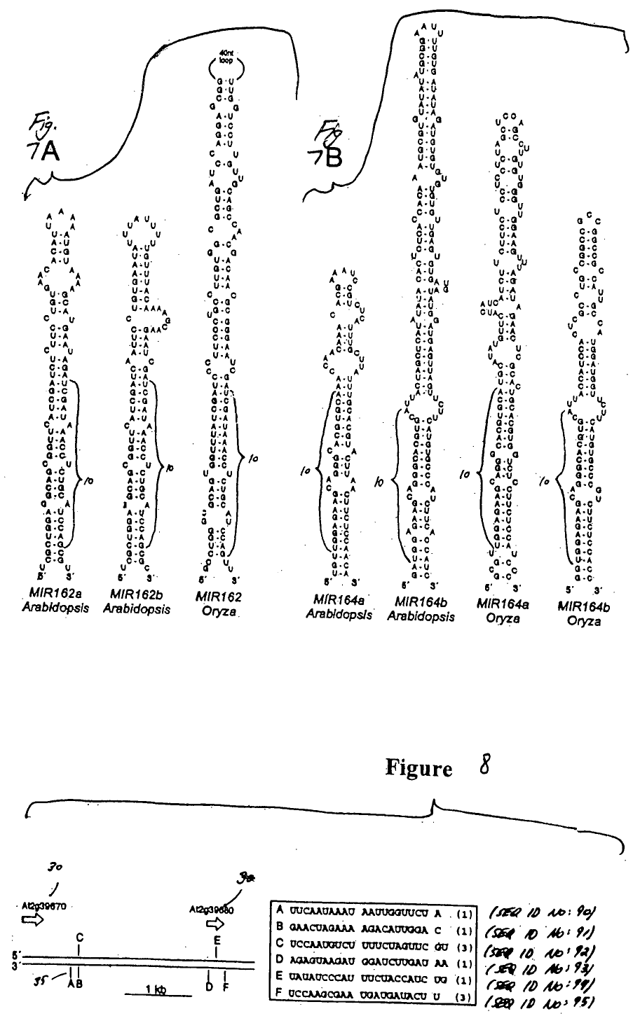

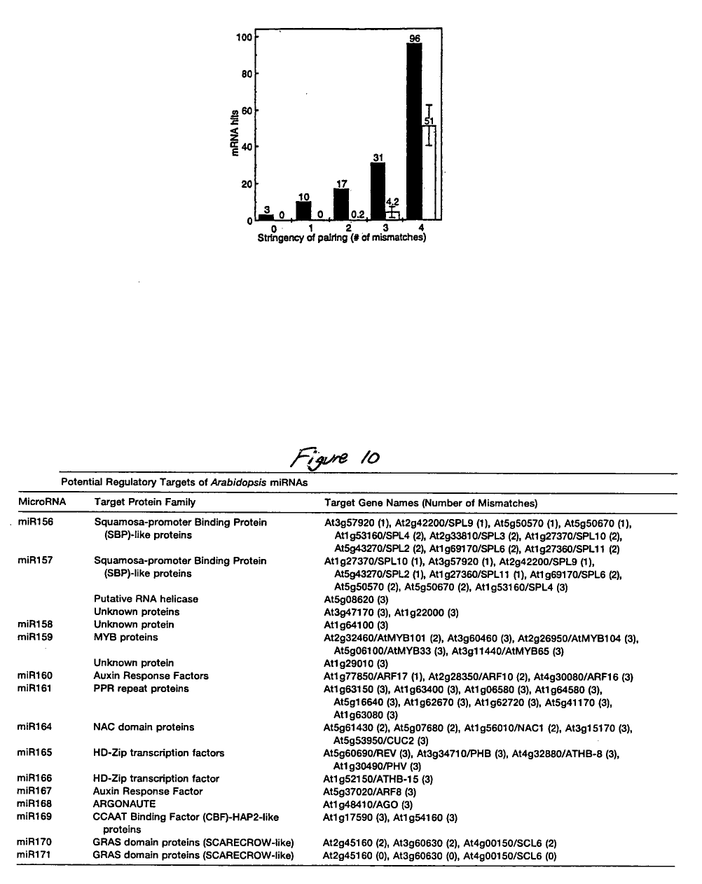

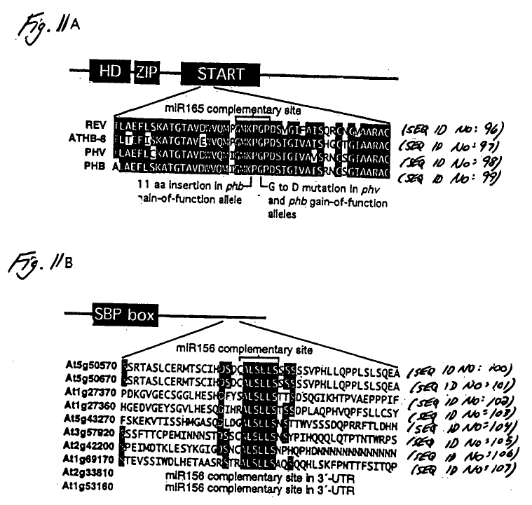

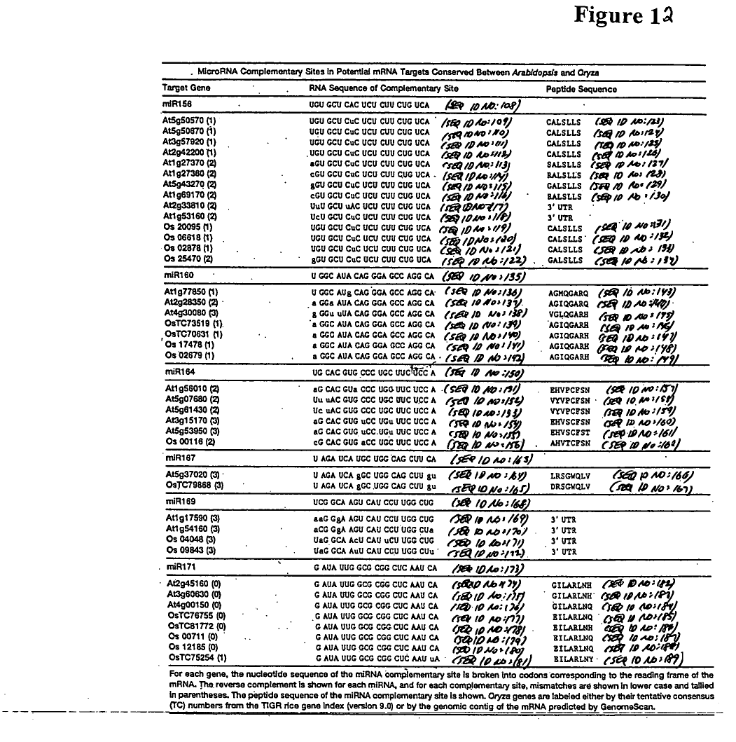

The location of the miRNA complementary sites within the mRNAs was conserved between Arabidopsis and rice. Importantly, when there were differences between Arabidopsis and rice complementary sites within homologous genes, these differences were distributed evenly across the three codon positions (FIG. 12). Homologous regions under selection only at the protein level tended to exhibit a higher frequency of differences at codon position 3. Thus, the substantially even distribution of mismatches across the codon positions indicated selection occurring at the nucleic acid level, in addition to any selection at the protein level, as would be expected if these segments act in miRNA recognition.

In FIG. 12, for each gene, the nucleotide sequence of the miRNA complementary site is broken into codons corresponding to the reading frame of the mRNA. The reverse complement is shown for each miRNA, and for each complementary site, mismatches are shown in lower case and tallied in parentheses. The peptide sequence of the miRNA complementary site is shown. Oryza genes are labeled either by their tentative consensus (TC) numbers from the TIGR rice gene index (version 9.0) or by the genomic contig of the mRNA predicted by GenomeScan.

Further evidence that these genes are regulatory targets of the miRNAs is provided by the identity of the genes themselves. Sixty-one miRNA/mRNA pairings are reported, which, due to overlap between similar miRNAs, represent 49 unique genes (FIG. 10). Of these 49 predicted targets, 34 are known or putative transcription factors (FIG. 10), even though transcription factors are thought to represent only 6% of protein-coding genes in Arabidopsis. Many of these genes specify shoot and floral meristem development or, for those with unknown functions, are in families that have members involved in meristem development. For example, the predicted targets of miR164 include CUPSHAPED COTYLEDON2 (CUC2), which is required for shoot apical meristem formation, and miR165 predicted targets include PHABULOSA (PHB) and PHAVOLUTA (PHV), which encode HD-Zip transcription factors that regulate axillary meristem initiation and leaf development. A miR159 predicted target, AtMYB33, can bind to the promoter of the floral meristem identity gene LEAFY. Homologs of the SBPs, which are thought to regulate the Antirrhinum floral meristem identity gene SQUAMOSA (Klein et al., 1996), may in turn be regulated by miR156 and miR157.

Genetic evidence supports the regulatory roles of miR165 complementary sites within PHB and PHV (FIG. 11A). Multiple gain-of-function alleles have been isolated for both genes, and each of these mutations disrupts the miR165 complementary site, usually as a single-nucleotide substitution. In the mutant examined, phb mRNA expression extends more broadly than in wild-type, suggesting that complementarity to miR165 is required for confining PHB mRNA accumulation to the proper cell types.

A connection between miRNAs and meristem development is consistent with the phenotypes of the Arabidopsis carpel factory (caf) mutant. Dicer and CAF are homologous RNAseIII domain proteins required for the accumulation of mature miRNAs in animals and plants, respectively. Mutant alleles of CAF, which is also known as SHORT INTEGUMENT1 (SIN1), delayed the meristem switch from vegetative to floral development and cause overproliferation of the floral meristem. Other genes required for miRNA accumulation in animals are homologs of the Arabidopsis gene ARGONAUTE (AGO1), which is required for axillary shoot meristem formation and leaf development in Arabidopsis. While AGO1 has not previously been reported to influence miRNA accumulation in plants, it is a predicted target of miR168 (FIG. 10), suggesting a negative-feedback mechanism for controlling expression of the AGO1 gene.

Other predicted targets of miRNAs do not have direct roles in meristem identity, but rather may have roles in cell division or differentiation. For example, miR160 and miR167 were predicted to target auxin response factors, DNA binding proteins thought to control transcription in response to the phytohormone auxin. Transcriptional regulation may be important for many of the diverse developmental responses to auxin signals, which include, for instance cell elongation, division, and differentiation in both roots and shoots. The predicted targets--of miR170 and miR171 are three SCARECROW-like proteins, a family of transcription factors whose members have been implicated in radial patterning in roots, signaling by the =phytohormone gibberellin, and light signaling. Overall, the high percentage of predicted miRNA targets that acted as developmental regulators suggested that miRNAs were involved in a wide range of cell division and cell fate decisions throughout the plant.

Other experiments were conducted to show that this computational approach could also identify miRNA targets in C. elegans and D. melanogaster. In both organisms, the miRNAs had few mRNA hits with complementary sites, essentially the same number of hits as seen for randomized cohorts (data not shown). While the possibility that a few animal miRNAs do recognize their targets with near-perfect complementarity cannot be excluded, the general phenomenon of near-perfect complementarity appears to be specific to plants, and thus represents an important difference between plant and animal miRNAs. Two other differences emerge when comparing the predicted target sites of plant miRNAs with those of the C. elegans lin-4 and let-7 miRNAs. First, the plant complementary sites are primarily, though not, exclusively, within the ORFs (open reading frames), whereas the only proposed lin-4 and let-7 sites are within 3′ UTRs in animals. Second, multiple sites within the same target mRNA were not detected in plants, whereas there are typically multiple lin-4 and let-7 sites within each mRNA target.

These differences observed between plant and animal miRNA target recognition have certain mechanistic implications for plant miRNA function (FIGS. 13A-13B). Plant miRNA target recognition appears to resemble that of small interfering RNAs (siRNAs) much more than that of animal miRNAs. During RNA interference (RNAi), long double-stranded RNA is processed by Dicer into approximately 22 nucleotide siRNAs, which serve as guide RNAs to target homologous mRNA sequences for cleavage. Targeting either the ORF or the UTRs can be effective in certain cases, provided that the siRNA has near-perfect complementarity, or at least substantial complementarity, to the targeted mRNA. Plants also have siRNAs. Indeed, these tiny RNAs were first observed in plants and are associated with a process related to RNAi, known as posttranscriptional gene silencing (PTGS), which leads to the destruction of mRNA from plant viruses and trans-genes. Plant miRNAs resemble animal miRNAs in their biogenesis, in that they are derived from endogenous, evolutionarily conserved genes and are processed from stem-loop precursors by a Dicer homolog, with accumulation of mature miRNA from only one arm of the precursor stem-loop. However, plant miRNAs resemble siRNAs in their target recognition, suggesting that they may also resemble siRNAs in their mechanism of action (FIG. 13A). Thus, plant miRNAs may hybridize to mRNAs with near-perfect complementarity and target the mRNAs for cleavage, according to certain embodiments. A function in mediating RNA cleavage may allow the plant miRNAs to target any region of the mRNA (which can be used to inhibit genes in some cases), whereas the animal miRNAs that mediate translational attenuation may be relegated to 3′ UTRs in order to avoid the mRNA-clearing activity of ribosomes. The efficiency and finality of mRNA cleavage may require only a single complementary site in each message, whereas the regulatory mechanism of lin-4 and let-7 miRNAs, which leaves the mRNA intact, may generally require multiple target sites.

The observation that many plant miRNAs potentially target the mRNAs of transcription factors involved development suggests that some miRNAs may function to clear key regulatory transcripts from certain daughter cell lineages (FIG. 13B). Through the action of miRNAs, these inherited mRNAs could be eliminated without relying on constitutively unstable messages, and any remaining transcription from these genes be neutralized.

### EXAMPLE 3

In this example, it was shown that Arabidopsis ago1 mutants have increased accumulation of mRNAs known to be targeted for cleavage by miRNAs. In hypomorphic ago1 alleles, this compromised miRNA function occurs without a substantial change in miRNA accumulation, whereas in null alleles it is accompanied by a drop in some of the miRNAs. Therefore, AGO1 acts within the Arabidopsis miRNA pathway, probably within the miRNA-programmed RISC, such that the absence of AGO1 destabilizes some of the miRNAs. It was also shown that targeting of AGO1 mRNA by miR168 is needed for proper plant development, illustrating the importance of feedback control by this miRNA. Transgenic plants expressing a mutant AGO1 mRNA with decreased complementarity to miR168 overaccumulated AGO1 mRNA and exhibit developmental defects partially overlapping with those of dcl1, hen1, and hyl1 mutants, showing a decrease in miRNA accumulation. miRNA targets overaccumulated in miR168-resistant plants, showing that a large excess of AGO1 protein interfered with the function of RISC and/or sequesters miRNAs and/or other RISC components. Moreover, it was also shown that developmental defects induced by a miR168-resistant AGO1 mRNA could be rescued by compensatory miRNA that was substantially complementary to the mutant AGO1 mRNA, showing the regulatory relationship between miR168 and its target as well as demonstrating the engineering of artificial miRNAs for plants.

In plants, mutants that lack the nuclear DICERLIKE1 (DCL1) protein are embryo-lethal, indicating that DCL1 is required for plant viability, at least during reproduction and/or at early stages of development. Partial loss-of-function dcl1 mutants exhibiting point mutations in the RNA helicase domain (dcl1-7, dcl1-8) or truncation of the second dsRNA-binding domain (dcl1-9) are viable, but show developmental defects including sterility. Accompanying these defects are greatly reduced miRNA levels, suggesting the crucial role of miRNAs during plant development and reproduction. The proper accumulation of miRNAs also depends on the activity of two other nuclear proteins: HEN1 and HYL1. hen1- and hyl1-null alleles show reduced miRNA levels and developmental defects that overlap with that of partial loss-of-function dcl1 mutants. However, in contrast to dcl1 -null alleles, hen1- and hyl1-null alleles are viable. This suggests either that HEN1 and HYL1 do not have essential functions in the miRNA pathway or that other genes encode proteins with partially redundant functions.

Two other proteins may play a role in the plant miRNA pathway: HASTY (HST) and ARGONAUTE 1 (AGO1). HST encodes a protein that is homologous to Exportin-5, and hst mutants have developmental phenotypes, which may suggest that HST participates in the transport of miRNAs or precursor miRNAs from the nucleus to the cytoplasm. AGO1 is the founding member of the ARGONAUTE protein family, which comprises 10 members in Arabidopsis. AGO1 may be specifically required for siRNA accumulation and DNA methylation triggered by sense transgenes (“S-PTGS”), but not inverted repeat transgenes (“IR-PTGS”). Thus, AGO1 may not be part of RISC, but rather, may act upstream from the mRNA degradation step in the S-PTGS pathway. S-PTGS and IR-PTGS are two branches of the PTGS pathway that may converge toward a common RISC that contains other AGO proteins.

One or multiple AGO proteins may be a component of the miRNA RISC that mediates cleavage of plant mRNAs. AGO1, ZIP/AGO7, and PNH/ZLL/AGO10 are examples because ago1, zip/ago7, and pnh/zll/ago10 mutations affect development. AGO1 may act in the miRNA RISC, owing to the possible feedback regulation of AGO1 mRNA by miR168 (Example 2). Among the ten known plant AGO mRNA homologs, only AGO1 has extensive complementary to miR168 or any of the other known miRNAs, suggesting that AGO1 may be the only member of the ARGONAUTE family that is regulated by an miRNA, just as DCL1, the Dicer family member known to be required for miRNA accumulation, is the only one of the four Dicer homologs known to be an miRNA target. 5′-RACE experiments have revealed that AGO1 mRNA fragments that terminate precisely at the predicted site of miR168-directed cleavage accumulate in wild-type plants, showing that miR168 directs the cleavage of AGO1 mRNA. Furthermore, the steady-state level of uncleaved AGO1 mRNA increases in flowers of hen1 mutants that show reduced accumulation of miR168, indicating that miR168-directed cleavage of AGO1 mRNA is important for AGO1 regulation.

In this example, the role of AGO1 in the miRNA pathway was demonstrated. AGO1 may act in RISC; however, other AGO proteins may also participate in the miRNA RISC, together with AGO1 or in cells where AGO1 is not present. It was also demonstrated in this example that the feedback regulation of AGO1 mRNA by the miRNA pathway through the action of miR168 is crucial for proper plant development. Thus, decreasing the complementarity of AGO1 mRNA with miR168 resulted in increased accumulation of AGO1 mRNA and developmental defects. It was also shown that these defects could be rescued by expressing a compensatory miRNA that was substantially complementary to the mutant AGO1 mRNA, which may prove the regulatory relationship between miR168 and its target. This demonstration was also an example of the engineering of artificial miRNAs.

The ago1-1, ago1-3, ago1-25, ago1-26, ago1-27, dcl1-9, hen1-4, and hyl1-2 mutants have been previously described in the′ literature. Plants were grown under cool-white light in long days (16 h of light, 8 h of dark) at 23° C. or short days (8 h of light, 16 h of dark) at 17° C.

The total RNA was extracted as described using established protocols, separated by denaturing 15% polyacrylamide gel electrophoresis, and blotted to a nylon membrane (Genescreen Plus; PerkinElmer Inc.). MicroRNA probes were prepared by end-labeling antisense oligonucleotides using T4 polynucleotide kinase (New England Biolabs). Blots were rehybridized with a probe complementary to U6.

RNA was extracted from mutant and wild-type siblings segregating from heterozygote parents grown in short days for 4 months. Poly(dT) cDNAs were made by using the Invitrogen cDNA firststrand synthesis system. Quantifications were performed on a Bio-Rad IQcycler apparatus with the Quantitech SYBR green kit (QIAGEN) upon recommendations of the manufacturer. PCR was carried out in 96-well optical reaction plates heated for 10 minutes to 95° C. to activate hot start Taq DNA polymerase, followed by 50 cycles of denaturation for 30 seconds at 95° C. and annealing-extension for 45 seconds at 60° C. Target quantifications were performed with specific primer pairs designed for each side of the cleavage site by using Beacon Designer from Biosoft. Primers used for At1g27370/SPL10, At3g11440/MYB65, At1g77850/ARF7, At1g06580/PPR, At5g53950/CUC2, At5g37020/ARF8, At1g48410/AGO1, At3g60630/SCL6-III, and At3g18780/ACTIN2 have also been described in the literature. The primers used to quantify additional mRNAs are At1g01040/DCL1, 5′-GATCCATTCCTAAGCGAAGTTTCAGAG-3′ (SEQ ID NO: 196) and 5′-GCCCGAGCAACATAAAGATCCATAG-3′ (SEQ ID NO: 197); At1g30490/PHV, 5′-AGACCTTGGCGGAGTTCCTTTG-3′ (SEQ ID NO: 198) and 5′-GTTGCGTGAAACAGCTACGATACC-3′ (SEQ ID NO: 199); At1g429701GAPDH, 5′-TCTTTCCCTGCTCAATGCTCCTC-3′ (SEQ ID NO: 200) and 5′-TTTCGCCACTGTCTCTCCTCTAAC-3′ (SEQ ID NO: 201); At5g60390/eEF-1(A4), 5′-CTGGAGGTTTTGAGGCTGGTAT-3′ (SEQ ID NO: 202) and 5′-CCAAGGGTGAAAGCAAGAAGA-3′ (SEQ ID NO: 203).

For each cDNA synthesis, quantifications were made in triplicate. For each quantification, conditions were, as recommended, 1≧E≧0.85 and r²≧0.985, where E is the PCR efficiency and r² corresponds to the correlation coefficient obtained with the standard curve. For each quantification, a melt curve was realized at the end of the amplification experiment, using steps of 0.5° C. from 55° C. to 95° C., to ensure that quantification was not caused by primer self-amplification, but by a pure and common PCR product. Results were normalized to that of ACTIN2, then to the value of the isogenic wild-type sibling. For each mutant analyzed, results were considered as acceptable if the variation between the wild-type sibling and a true wild-type plant was <15%.

A KpnI-SalI fragment carrying the 3′-half of the AGO1 gene was subcloned from BAC F11A17 (position 12600-18030) into the binary vector pBin+. The 5′-half of the AGO1 gene was amplified by PCR from BAC F11A17 using the following pair of primers: 5′-CTCGACTCTCGAGGTAGTATTAATTAACGAGTTCTAAGTTCTTCTTCCGTTATGAG-3′ (SEQ ID NO: 204) and 5′-GGTTCTGGTACCTGGGTAGGACTCACCTCAGACAGTGTAGGCTGAGAAGACACCGC-3′ (SEQ ID NO: 205), cut with XhoI and KpnI (at positions 10,050 and 12,600 on BAC F11A17) and cloned into the Bluescript vector pKS+. The 5′-primer introduced a PacI site downstream from the XhoI site so that the 5′-half of the AGO1 gene can be mobilized as a PacI-KpnI fragment and cloned into the pBin+vector containing the 3′-half of the gene to reconstitute a complete AGO1 gene (WT-AGO1). Silent mutations were introduced into the miR168 complementary site using the Quick Change Site-Directed Mutagenesis Kit (Stratagene) and the following pair of primers: 5′-CCACCGCAGAGACAATCAGTGCCGGAGCTCCATCAGGCTACCTCACCTACTTATCA AGCG-3′ (SEQ ID NO: 206) and 5′-CGCTTGATAAGTAGGTGAGGTAGCCTGATGGAGCTCCGGCACTGATTGTCTCTGCG GTGG-3′ (SEQ ID NO: 207). The 4m-AGO1 construct resulted from the perfect replacement of the wild-type sequence by the primer sequence, whereas the 2m-AGO1 construct resulted from partial replacement of the wild-type sequence by the primer sequence. The wild-type and mutagenized PacI-KpnI fragments were entirely sequenced to ensure that no other mutations have been introduced and transferred from pKS+ into the pBin+ vector containing the 3′-half of the gene to reconstitute a complete AGO1 gene.

The MIR168a gene was subcloned from BAC T5K18 into the Bluescript vector pKS+ as a PstI-ClaI fragment (position 55287-57718 on T5K18). Compensatory mutations that restore complementarity to the 4m-AGO1 mRNA were introduced into the MIR168a gene using the Quick Change Site-Directed Mutagenesis Kit (Stratagene). The miR168 sequence was first mutageneized using the following pair of primers: 5′-CACCATCGGGCTCGGATTCGCCTGGTGGAGGTCCGGCACCAATTCGGCTGACACAG CC-3′ (SEQ ID NO: 208) and 5′-GGCTGTGTCAGCCGAATTGGTGCCGGACCTCCACCAGGCGAATCCGAGCCCGATGG TG-3′ (SEQ ID NO: 209). The miR168*sequence was subsequently mutageneized using the following pair of primers: 5′-TTGGTfTGTGAGCAGGGATTGGAGCCGGCCTTCCATCAGCTGAATCGGATCCTCGAG GTGTA-3′ (SEQ ID NO: 210) and 5′-TACACCTCGAGGATCCGATTCAGCTGATGGAAGGCCGGCTCCAATCCCTGCTCACA AACCAA-3′ (SEQ ID NO: 211). The mutagenized PstI-ClaI fragment was sequenced to ensure that no other mutations have been introduced and then was transferred from pKS+ into the pCambia1200 binary vector.

The WT-AGO1 , 2m-AGO1, and 4m-AGO1 constructs (in pBin+) and the 4m-MIR168a construct (in pCambia1200) were transferred from Escherichia coli to Agrobacterium tumefaciens by triparental mating. Arabidopsis plants were transformed by the flower-dipping method. Transformants were selected by sowing seeds onto a medium supplemented with kanamycin (pBin+) or hygromycin (pCambia1200).

Three alleles (ago1-22, ago1-23, ago1-24) exhibited a phenotype similar to that of the ago1-3-null allele previously identified through a phenotypic screen for developmental mutants (FIG. 14). In FIGS. 14A-F, rosettes of plants grown under short-day conditions are shown, while in FIGS. 14G-14L are flowers of plants grown under long-day conditions are shown. The mutants are indicated in the upper right corner of each figure. Three other alleles (ago1-25, ago1-26, ago1-27) exhibited less dramatic developmental defects, although they were deficient for PTGS, suggesting that PTGS is more sensitive to perturbation in AGO1 than is development. Additionally, ago1 hypomorphic mutants exhibited plant stature, leaf shape; and flower phenotypes partially overlapping those of dcl1, hen1, or hyl1 mutants, which were impaired in the accumulation of miRNAs (FIG. 14), showing that AGO1 may play a role in the miRNA pathway.

In the dcl1, hen1, and hyl1 mutants, the impairment of the miRNA pathway resulted in increased steady-state levels of uncleaved target mRNAs or decreased steady-state levels of cleavage products, with the exception of AP2 mRNA, for which regulation by miR172 relied on translational repression. To determine if AGO1 also participates in the regulation of endogenous mRNAs by miRNAs, steady-state levels of 10 mRNAs targeted for cleavage by miRNAs of 10 different families were quantified. As controls, the level of GAPDH and eEF-1 (A4) mRNA were also quantified, which are not believed to be targeted by miRNAs. Because it was not possible to extract enough RNA from flowers of ago1-null alleles, the mRNA steady-state levels in rosettes of representative ago1 mutants were analyzed: ago1-27 (hypomorphic), ago1-26 (intermediate), ago1-3 (null), as well as hen1-4- and hyl1-2-null alleles (all in the Col ecotype) grown in short days.

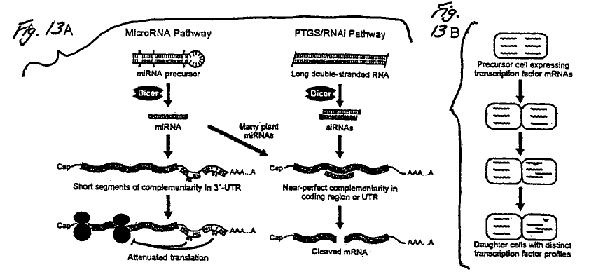

A limited increase in the accumulation of miRNA targets was observed in hen1, hyl1, and ago1 hypomorphic mutants (FIGS. 15A-15L), with each target responding to a different extent in the different genetic backgrounds as previously reported in leaves or flowers of dcl1, hen1, and hyl1 mutants. In FIGS. 15A-15L, RNA extracted from rosettes of isogenic wild-type or mutant siblings deriving from heterozygote parents and of untransformed plants or 2m-AGO1 transformants was quantified for the indicated mRNA by real-time quantitative PCR using primers surrounding the cleavage site. GAPDH and eEF-1(A4) were used as nontarget controls. Quantifications were normalized to that of ACTIN2, then to the value of the wild-type plants or wild-type siblings, which was arbitrarily fixed to 1. A consistent and stronger increase in the accumulation of miRNA targets but not of GAPDH and eEF-1 (A4) mRNAs was observed in the ago1-3-null allele, which was confirmed on a second set of independent plants (data not shown). Most of the miRNA targets analyzed are expressed at much higher levels in meristem than in leaves. Because ago1-null alleles develop smaller leaves than hen1, hyl1, and ago1 hypomorphic mutants (FIGS. 14A-14F), the ratio between meristematic cells and leaf cells in a rosette is probably higher in ago1-null alleles, thus introducing a possible bias in the analysis. Nevertheless, the similar increase observed in hen1, hyl1, and ago1 hypomorphic mutants, which all exhibit similar development, indicated that miRNA-directed mRNA cleavage involves AGO1.

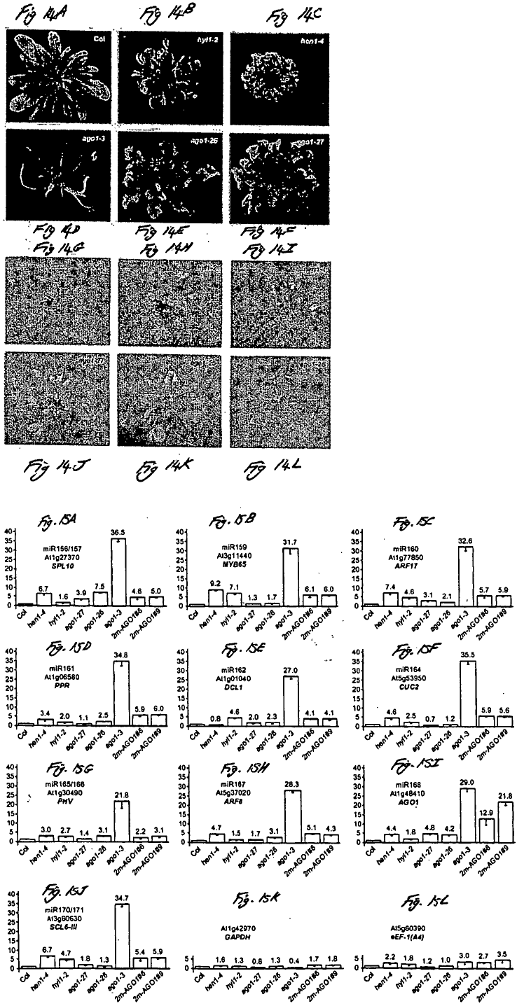

The increased accumulation of target mRNAs observed in dcl1, hen1, and hyl1 mutants may be caused by reduced miRNA accumulation and subsequent reduced cleavage efficiency. To examine at which step ago1 mutants were impaired in the miRNA pathway, miRNA accumulation in rosettes of wild-type plants-and ago1 mutants was analyzed using the same plant material used for the RT-qPCR experiments. It was observed that miRNAs were present in ago1-27 and ago1-26 at levels similar to those observed in wild-type plants (FIG. 16A), suggesting that AGO1 acted downstream from DCL1, Hen1, and HYL1 in the miRNA pathway to promote cleavage of target mRNAs. In the ago1-3-null allele, only miR156/157 and miR167 accumulated to a level similar to that of wild-type plants (FIG. 16B). For the eight other miRNAs examined, accumulation in ago1-3 was reduced, in some cases to below the level of detection. Thus, AGO1 may be important for the stabilization of miRNAs, although it may also have a role in miRNA production. It seems likely that Arabidopsis AGO1 functions similarly to Drosophila AGO2 or human eIF2C, which associate with miRNAs in the RISC, which, in turn, mediates the posttranscriptional regulation of target messages. Alternatively, AGO1 may act before miRNA processing, as suggested by the recent observation that the precursor of miR165 is mislocalized in ago1 mutants.

The results presented in FIG. 15 show that the steady-state level of uncleaved AGO1 mRNA was increased in hen1, hyl1, and ago1 mutants, strongly supporting the idea that AGO1 mRNA undergoes a negative feedback regulation by the miRNA pathway through the action of miR168. FIG. 15 shows miRNA accumulation in ago1 mutants, where miRNA accumulation was determined by RNA gel blot analysis using 30 micrograms (FIG. 15A) or 10 micrograms (FIG. 15B) of the same RNA used for RT-qPCR analyses. Blots were successively hybridized to different probes complementary to miRNAs. FIG. 15A shows miRNA accumulation in the ago1-26 and ago1-27 hypomorphic alleles. FIG. 15B shows miRNA accumulation in the ago1-3-null allele. If the negative feedback regulation of AGO1 mRNA by the miRNA pathway is essential to maintain a proper regulation of plant development by miRNAs, plants impaired in this feedback regulation but expressing functional AGO1, DCL1, Hen1, and HYL1 proteins should exhibit developmental defects. To test this hypothesis, silent mutations were introduced in the AGO1 gene to decrease the complementarity between AGO1 mRNA and miR168 without changing the AGO1 protein sequence. In the first step, the wild-type AGO1 gene was subcloned as an 8 kb fragment carrying the entire transcribed region plus 1.5 kb upstream of the transcription start and 0.5 kb downstream from the polyadenylation signal. Introduction of this construct (WTAGO1) into the ago1-27 hypomorphic allele or the ago1-]-null allele restored a wild-type phenotype in about 80% of the transformants, indicating that the WT-AGO1 construct contained the upstream and downstream regulatory elements required for wild-type function. Introduction of WT-AGO1 into wild-type plants had no effect on plant development in 94 out of the 131 transformants analyzed. The remaining 37 transformants looked normal at early stages of development but progressively exhibited the characteristics of ago1 hypomorphic alleles, including late flowering, serrated leaves, fused flowers, and limited fertility (data not shown), suggesting that the WT-AGO1 construct had triggered late co-suppression of the endogenous AGO1 gene. To test this hypothesis, the WT-AGO1 construct was introduced into the co-suppression-deficient sgs2 mutant. None of the 130 transformants analyzed showed an ago1 hypomorphic phenotype, thus confirming that the phenotype observed in transformed wild-type plants resulted from co-suppression of the endogenous AGO1 gene.

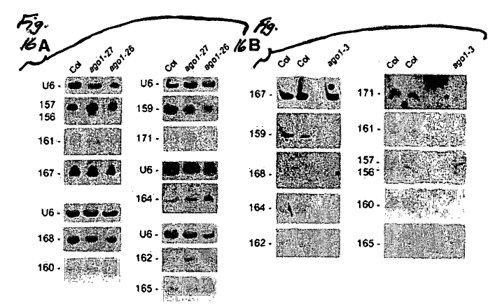

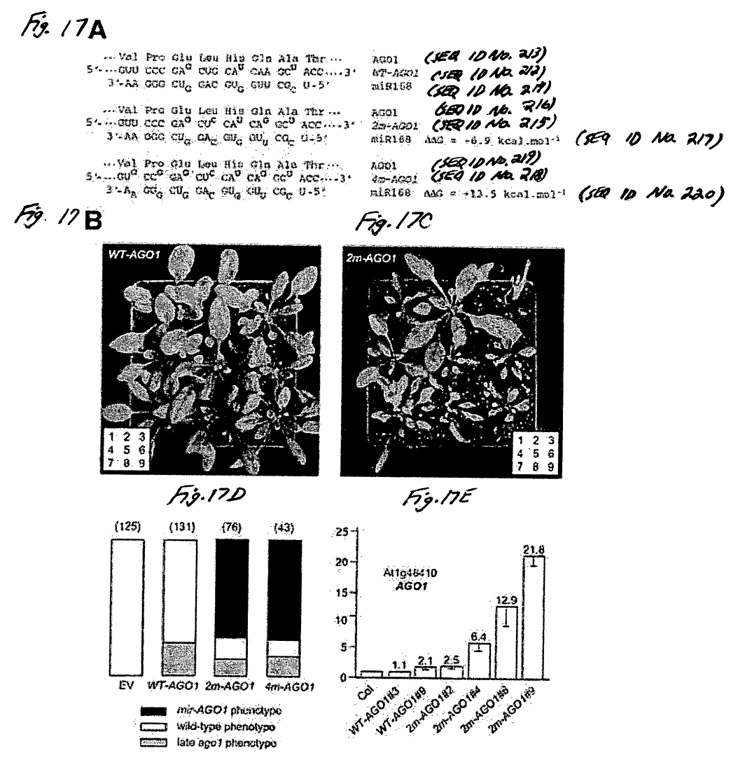

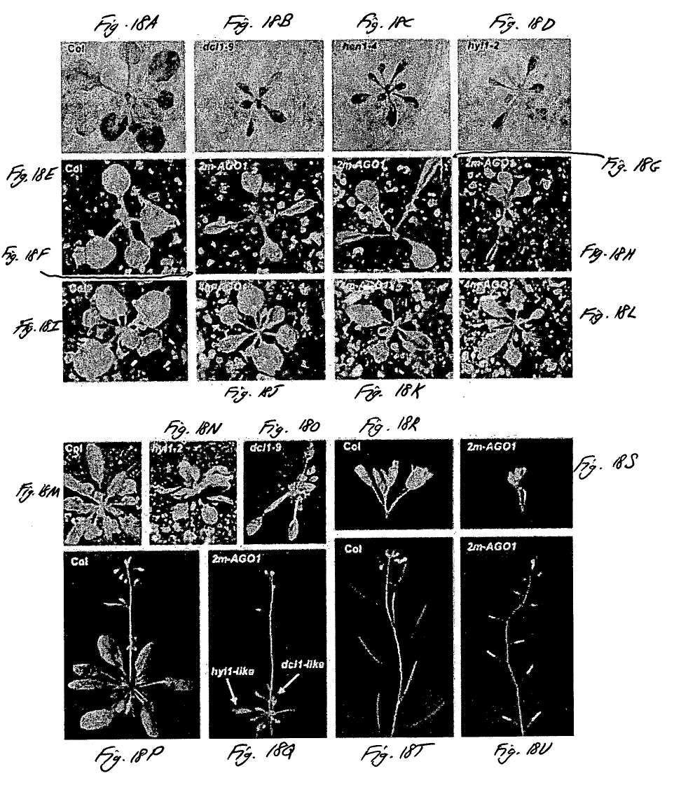

The wild-type AGO1 gene encodes an mRNA that naturally contains three mismatches with miR168 (Example 2). Two mutant constructs (2m-AGO1 and 4m-AGO1) were made by introducing two or four silent mutations into the WT-AGO1 construct, thus adding two or four mismatches with miR168 that increased the free energy of the miRNA/target duplex by 6.9 kcal/mole and 13.5 kcal/mole, respectively (FIG. 17A). When introduced into wild-type plants, the 2m-AGO1 and 4m-AGO1 constructs yielded 13% to 18% of transformants with a cosuppressed phenotype similar to the 28% of cosuppressed transformants observed with the WT-AGO1 construct (FIG. 17D). In addition, the 2m-AGO1 and 4m-AGO1 constructs yielded 63% to 68% of transformants exhibiting developmental defects (referred to as the miR-resistant AGO1 or mir-AGO1 phenotype) that were not observed in plants carrying the WT-AGO1 construct (FIG. 17B-17D).

Silent mutations in the miR168 complementary site of the AGO1 mRNA induced developmental defects, illustrated in FIGS. 17A-17E. In FIG. 17A, the WT-AGO1 mRNA naturally contains three mismatches with miR]68 (indicated by*'s), including a G:U wobble pair. Silent mutations in 2m-AGO1 and 4m-AGO1 constructs introduce two and four additional mismatches (indicated by x's), reducing complementarity with miR168. ΔΔG was calculated using mfold. FIGS. 17B and 17C indicate representative sets of transformants carrying the WT-AGO1 or 2m-AGO1 construct. FIG. 17D shows the proportion of transformants showing a wild-type phenotype (open bars), an ago1 phenotype caused by late co-suppression (shaded bars), or an mir-AGO1 phenotype caused by AGO1 overexpression (black bars). Plants were transformed with either an empty vector (“EV”) or the WT-AGO1, 2m-AGO1, or 4m-AGO1 constructs. The number of transformants analyzed is indicated in parentheses. FIG. 17E shows AGO1 mRNA accumulation determined by real-time quantitative PCR in untransformed plants (Col) or plants transformed with the WT-AGO1 or 2m-AGO1 constructs. Quantifications were normalized to that of ACTIN2. The value in Col was arbitrarily fixed to 1. Numbers correspond to the plants shown in B.

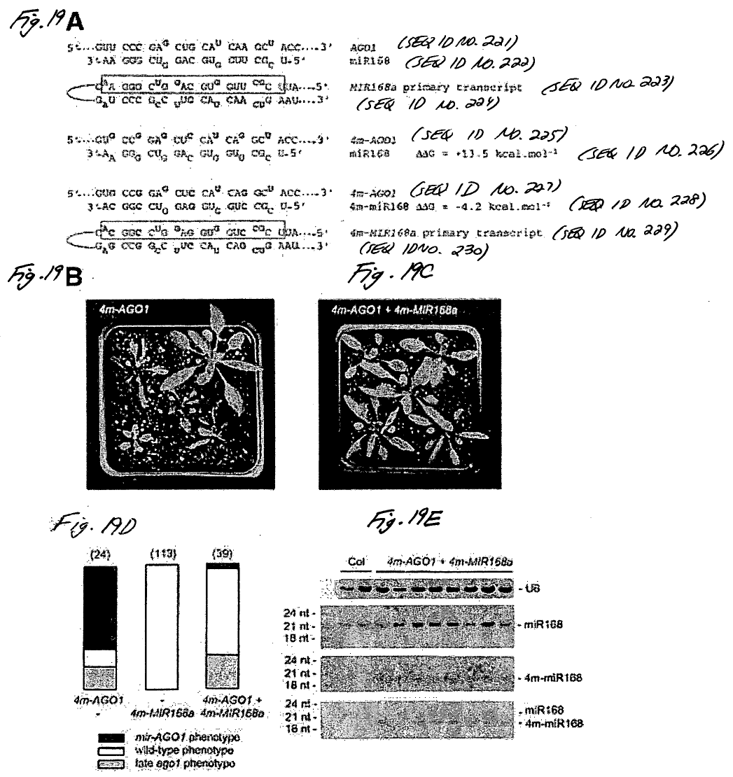

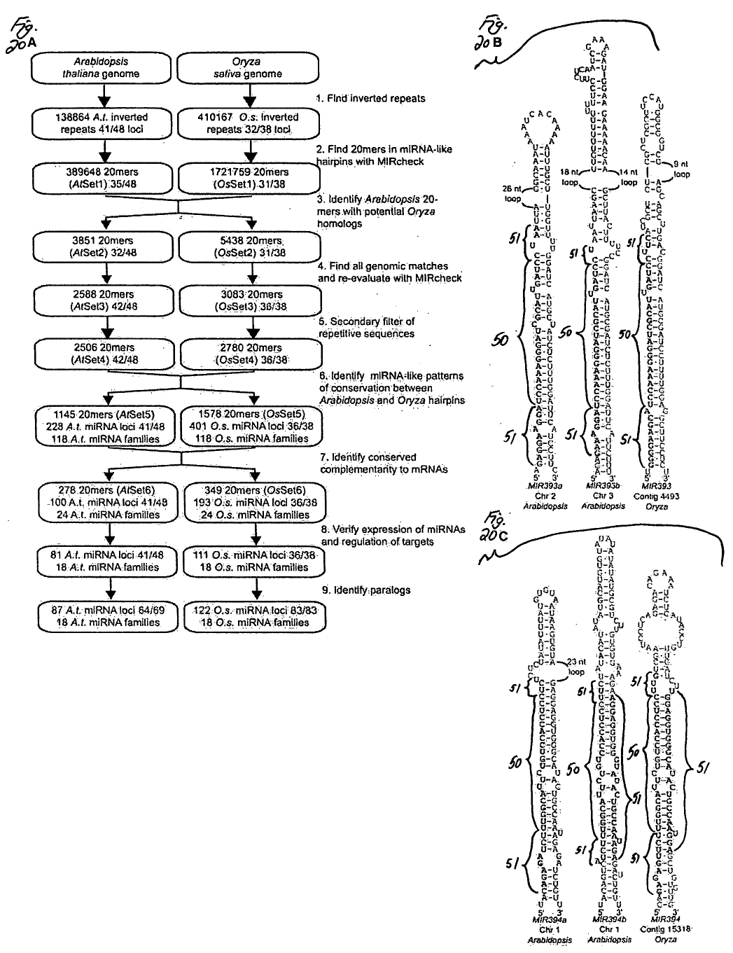

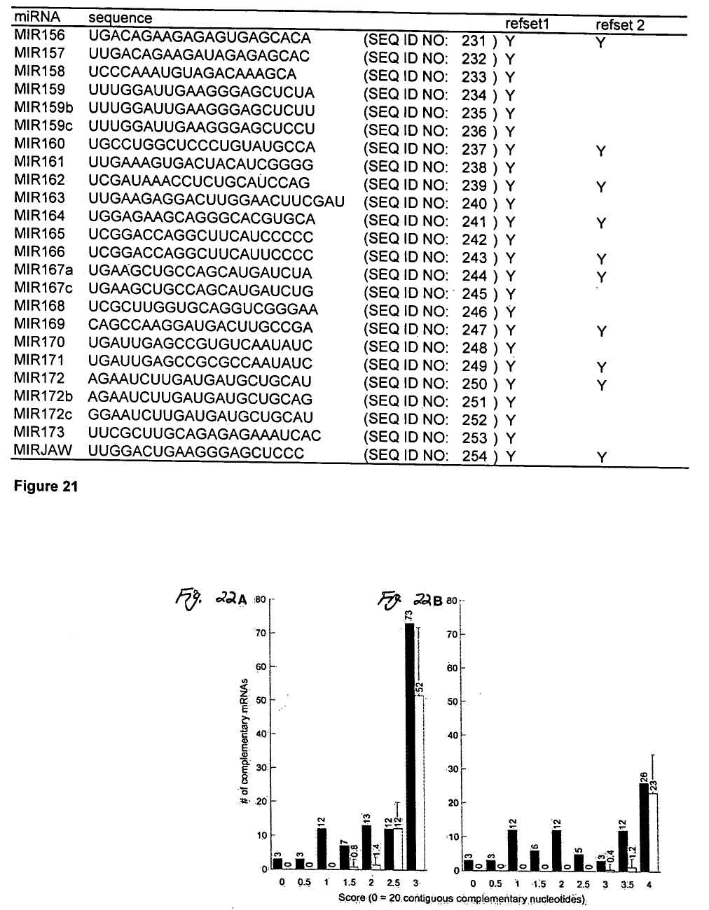

When introduced into the co-suppression-deficient mutant sgs2, the 4m-AGO1 construct yielded 91% of transformants with the mir-AGO1 phenotype, and no transformants with the cosupppression phenotype (data not shown). In young seedlings, the mir-AGO1 phenotype was mostly characterized by the emergence of curled leaves resembling those of hen1 and hyl1 mutants (FIG. 18A-L). Additional developmental defects were also observed, including abnormal cotyledons. As plants grew, developmental defects became more variable from plant to plant and from one leaf to another (FIG. 18A-L). Adult transformants exhibited a shorter stature, asymmetric rosette leaf formation, twisted or spoon-shaped leaves resembling those observed in hyl1 and dcl1 mutants, respectively, a disorganized phyllotaxy, and an accelerated senescence of leaves exemplified by photobleaching spots on a highly anthocyaned background (FIGS. 17B, 18M-18Q). In the most affected transformants, the shoot apical meristem aborted and the plants died before flowering. In less severely affected transformants, a short stem developed bearing degenerate flowers that were mostly sterile (FIG. 18R-18U). The rest of the transformants produced seeds, the amount of which inversely correlated with the severity of the developmental defects observed in the vegetative phase.

FIG. 18A-18Q illustrates developmental defects in 2m-AGO1 transformants. FIGS. 18A-18D show wild-type plant (Col) and dcl1, hen1, and hyl1 mutants. FIGS. 18E-18H show wild-type plant (Col) and 2m-AGO1 transformants exhibiting curled leaves resembling those of hen1 and hyl1 mutants, at 10 days. Transformants with aberrant cotyledons were also occasionally observed. FIGS. 18I-18L show wild-type plant (Col) and 4m-AGO1 transformants exhibiting a variety of developmental defects, including asymmetric rosette leaf formation and curled or twisted leaves, at 19 days. FIGS. 18M-18O show wild-type plant (Col) and dcl1 and hyl1 mutants. FIGS. 18P and 18Q show adult wild-type plant (Col) and a representative 2m-AGO1 transformant exhibiting spoon-shaped or twisted anthocyaned leaves resembling those of dcl1 and hyl1 mutants. FIGS. 18R and 18S show inflorescence of a wild-type plant (Col) and of a representative 2m-AGO1 transformant. FIGS. 18T-18U show stems and siliques (seed pods) of the same plants. The wild-type Col plant was fertile, whereas the 2m-AGO1 transformant was sterile with aborted siliques.

In the progeny of fertile transformants exhibiting a weak phenotype, a 3:1 ratio of abnormal/wild-type plants was observed, consistent with the expected dominant character of constructs triggering ectopic gene expression. This result also indicated that developmental changes induced by 2m-AGO1 or 4m-AGO1 constructs were reversible by segregation of the constructs at meiosis and did not induce inherited imprints, consistent with the posttranscriptional level of deregulation induced by the constructs. Transformants exhibiting more severe phenotypes produced small siliques containing either no seeds or very few seeds. In the progeny of such transformants, the ratio of abnormal/wild-type plants was less than 3:1 and, in the case of the most affected fertile transformant, declined to 1:5. This deficit in abnormal plants showed that expression of the 2m-AGO1 or 4m-AGO1 construct may compromise gamete or embryo viability. This is consistent with the lower number of transformants generated with the 2m-AGO1 construct, compared with the WT-AGO1 construct, and the even lower number obtained with the 4m-AGO1 construct (FIG. 17D). Because the T-DNA stably integrates into mature female gametes during the floral dipping procedure used to transform Arabidopsis, only the transformed embryos that do not express the 2m-AGO1 or 4m-AGO1 construct at high levels could survive and develop into seeds.

The accumulation of uncleaved AGO1 mRNA was determined by RT-qPCR in rosettes of representative transformants (FIG. 17E). No difference or a slight increase of 2.1-fold was observed between wild-type plants and transformants carrying the WT-AGO1 construct (WTAGO1#3 and #9). A limited increase (2.5-fold and 6.4-fold) was observed in 2m-AGO1 transformants that did not exhibit a strong phenotype (2m-AGO1 #2 and #4). A stronger increase (12.9-fold and 21.7-fold) was observed in 2m-AGO1-transformants exhibiting a strong phenotype (2m-AGO1 #6 and #9). These results are consistent with RT-qPCR analyses showing that AGO1 mRNA strongly overaccumulated in the ago1-3 mutant, in which miRNA-mediated cleavage was generally impaired (FIGS. 15A-15L), which confirms that miR168 regulated the AGO1 mRNA level through cleavage. They also indicated that plants can tolerate a limited increase in the amount of AGO1 mRNA accumulation without dramatically affecting development, whereas a strong increase in the amount of AGO1 mRNA triggered dramatic developmental defects.

Because 2m-AGO1 and 4m-AGO1 transformants exhibited spoon-shaped, curled or twisted leaves resembling those of dcl1, hen1, and hyl1 mutants (FIGS. 18A-18Q), the steady-state level of mRNAs targeted for cleavage by miRNAs were analyzed in rosettes of two 2m-AGO1 transformants exhibiting a strong phenotype (2m-AGO1 #6 and #9). For every miRNA target examined, the level in 2m-AGO1 transformants was slightly higher than that in wild-type plants (FIG. 15A-15L), showing that an increase in the amount of AGO1 perturbates the miRNA pathway. The excess of AGO1 protein may interfere with the formation or the functioning of RISC by displacing other AGO proteins. Alternatively, the excess of free AGO1 protein may independently titrate miRNAs and/or other RISC components into separate incomplete complexes. Such a sequestration of miRNAs-and/or other RISC components by free AGO1 protein may therefore mimic the effect of dcl1, hen1, or hyl1 mutations that reduce the accumulation of miRNAs, thus leading to some of the developmental defects.

To show that the developmental abnormalities observed in 4m-AGO1 transformants resulted from the absence of regulation of 4m-AGO1 mRNA by miR168, the MIR168a gene was mutagenized to introduce compensatory mutations that would allow the production of an miRNA that can pair with the mutant AGO1 mRNA transcribed from the 4m-AGO1 construct. miR168 is processed from an imperfect hairpin in which 15 of 21 miRNA residues are predicted to take part in Watson-Crick pairs involving residues on the other arm of the hairpin (FIG. 19A). When the AGO1 gene was altered to introduce mutations that decrease the complementarity with miR168, nucleotides were chosen that kept the amino acid sequence unchanged and also corresponded to paired nucleotides in the miRNA precursor. Therefore, compensatory mutations could be introduced on both sides of the stem of the dsRNA precursor encoded by the MIR168a gene to generate a new miRNA precursor with the same predicted folding geometry as the original precursor (FIG. 19A). The MIR168a gene was used because of the existence of a corresponding EST starting 20 bp (base pairs) upstream of the fold-back stem-loop and ending 180 bp downstream, thus indicating that this gene is transcribed. The wild-type MIR168a gene was subcloned as a 2.4 kb-fragment with 14 kb upstream of and 1.0 kb downstream from the miR168 sequence so as to include regulatory sequences. The miR168 and miR168*sequences were successively mutagenized to generate the 4m-MIR168a construct (FIG. 19A). The predicted free energy of the 4m-miR168/4m-AGO1 duplex was slightly more favorable than that of the wild-type mir168/wt-AGO1 duplex (by 4.2 kcal/mole), suggesting that the newly generated miRNA would cleave the mutant 4m-AGO1 mRNA at least as well as the wild-type miRNA cleaves the wild-type AGO1 mRNA (FIG. 19A).

Wild-type plants were transformed with either 4m-AGO1 or 4m-MIR168a or cotransformed with both constructs (each carried on a different vector with unique selectable markers). The number of transformants carrying the two constructs was low because the frequency of double transformation by independent bacteria was low. Nevertheless, it was higher than that observed with the 4m-AGO1 construct alone, suggesting that the deleterious effect of the 4m-AGO1 construct was abolished in the presence of the 4m-MIR168a construct. Further, it was observed that only one out of 39 transformants carrying both 4m-AGO1 and 4m-MIR168a constructs displayed the mir-AGO1 phenotype, whereas 17 out of 24 transformants carrying the 4m-AGO1 construct alone displayed this phenotype (FIG. 19B-19D). To confirm the efficiency of the rescue, a larger number of wild-type plants were transformed with the two constructs. Only two out of 77 additional double transformants displayed the mir-AGO1 phenotype (data not shown). No phenotype was observed in plants transformed with the 4m-MIR168 construct alone (FIG. 19D). Double transformants that carry both 4m-AGO1 and 4m-MIR168a constructs and exhibited a wild-type phenotype accumulated 4m-miR168, whereas no signal was visible in untransformed plants (FIG. 19E), indicating that the probe does not cross-hybridize with the endogenous miR168 and that the signal corresponds to bona fide 4m-miR168. Rehybridization of the blot with a probe complementary to miR168 revealed that the level of accumulation of miR168 was not affected by the expression of 4m-miR168. Interestingly, 4m-miR168 migrated faster than miR168, probably by 1 nucleotide. These results show that the 4m-MIR168a construct was functional and expressed sufficient 4m-miR168 to rescue the phenotype conferred by the 4m-AGO1 construct. However, the observation that 4m-miR168 is shorter than miR168 by 1 nucleotide indicated that subtle changes in the duplex induced by changes in the primary sequence of the miRNA transcript may affect the boundaries of the cleavage.

FIGS. 19A-19E shows compensatory mutations in the MIR168a gene rescue developmental defects induced by silent mutations in the miR168 complementary site of the AGO1 mRNA. In FIG. 19A, the MIR168a gene encodes a primary transcript that is partially, paired. The miRNA is boxed. Compensatory mutations in the 4m-MIR168a transgene (x's); conserved the structure of the primary transcript and restored pairing with the 4m-AGO1 mRNA. Original mismatches (*'s) were kept. ΔΔG was calculated using mfold. FIGS. 19B and 19C show representative sets of transformants carrying the 4m-AGO1 construct alone or the 4m-AGO1 and 4m-MIR168a constructs together. FIG. 19D shows the proportion of transformants showing a wild-type phenotype (open bars), an ago1 phenotype caused by late co-suppression (shaded bars), or a mir-AGO1 phenotype caused by AGO1 overexpression (black bars). Plants were transformed with the 4m-AGO1 or 4m-MIR168a constructs, or both. The number of transformants analyzed is indicated in parentheses. FIG. 19E shows the accumulation of the compensatory miRNA (m4-miR168) in double transformants carrying the 4m-AGO1 and 4m-MIR168a constructs. RNA gel blot analysis was performed using 20 micrograms of total RNA extracted from two nontransformed plants (Col) and eight independent double transformants. The blot was hybridized with a probe complementary to 4m-miR168, stripped, rehybridized with a probe complementary to miR168, stripped, and finally rehybridized with the two probes simultaneously.

In this example, it was shown that Arabidopsis ago1 mutants that exhibit a range of developmental defects (FIG. 14A-14L) also exhibited increased accumulation of mRNAs targeted by miRNAs (FIGS. 15A-15L). miRNA accumulation is not affected in ago1 hypomorpohic alleles that retain a PAZ domain, but is reduced in ago1-null alleles (FIG. 16A-16B), supporting a role for AGO1 in the miRNA pathway. AGO1 may participate in the distribution of miRNAs or miRNA precursor transcripts, or could function in RISC, similar to eIF2C2 in human, AGO2 in Drosophila, or QDE-2 in Neurospora. However, it is unlikely that AGO1 is the only AGO protein that associates with miRNAs in RISC.

ago1-null alleles were typically viable, although they exhibited dramatic developmental defects (FIG. 14A-14L). This viability may be caused by the function of other AGO protein(s), in particular PINHEAD/ZWILLE, which is 75% similar to AGO1 and has a pattern of expression overlapping with that of AGO1. In addition, plants homozygous for both ago1-null and pnh-null mutations were embryo-lethal, pointing out the crucial roles of these two proteins in plant development. Interestingly, ago1 hypomorphic mutants resemble transgenic plants expressing the viral suppressor of PTGS HC-Pro. The molecular phenotypes were also similar. The overlapping physical and molecular phenotypes of ago1 mutants and HC-Pro transgenic plants shows that HC-Pro can alter the miRNA pathway by interfering with the action of AGO1.

Decreasing the complementarity of AGO1 mRNA-with miR168 increased the level of AGO1 mRNA (FIG. 17A-17E) and may have dramatic consequences on plant development and reproduction (FIG. 18A-18U). This observation, together with the finding that AGO1 is needed for proper miRNA function, supports a conclusion that AGO1 mRNA is subjected to negative feedback regulation through the action of miR168. In this scenario, if the amount of AGO1 activity in a wild-type cell decreases below a critical level, the efficiency of miR168-mediated cleavage of AGO1 mRNA would also decrease, allowing more AGO1 mRNA to be translated into AGO1 protein and restoring activity to the initial level. Of course, the miR162 regulation of DCL1 mRNA could also come into play, evoking more complex scenarios. For example, lowered AGO1 activity would decrease the amount of miR162-directed DCL1 mRNA cleavage, which could increase the amount of miRNAs produced, increasing the amount of AGO1 mRNA cleavage and further lowering AGO1 activity. Thus, the outcome of a change in AGO1 activity could depend on many factors, including which of the components, DCL1, AGO1, miR162, and miR168, are limiting or in excess at the time of the perturbation. Another consideration is that a large increase in AGO1 mRNA, and presumably AGO1 protein, results in an apparent decrease in RISC activity. Such a large increase may be attainable in a wild-type plant, or alternatively, marginal increases in AGO1 may be held in check by miR168 feedback repression. In transgenic plants expressing an mir168-resistant AGO1 mRNA, the system cannot return to the equilibrium, because the mutant AGO1 mRNA is insensitive to feedback regulation by miR168.

The expression of a mutant miRNA able to pair with the mutant AGO1 mRNA can restore the feedback regulation and rescue the developmental defects induced by the mutant AGO1 mRNA (FIG. 19A-19E). This phenotypic rescue proves the regulatory relationship between miR168 and its target. It also demonstrates the feasibility of engineering artificial miRNAs in plants. Thus, this example demonstrates their use as tools for targeted silencing of a gene or a gene family, as well as their utility for exploring facets of miRNA maturation and function.

### EXAMPLE 4

This example develops comparative genomic approaches to systematically identify both miRNAs and their targets in Arabidopsis thaliana and rice (Oryza sativa). In this example, 23 miRNAs, representing seven newly identified gene families, were experimentally validated in Arabidopsis. Nineteen newly identified target candidates were confirmed by detecting mRNA fragments diagnostic of miRNA-directed cleavage in plants. Overall, plant miRNAs have a strong propensity to target genes controlling development, particularly those of transcription factors and F-box proteins. As shown in this example, plant miRNAs also may have conserved regulatory functions extending beyond development., in that they also target superoxide dismutases, laccases, and ATP sulfurylases. The expression of miR395, the sulfurylase-targeting miRNA, increases upon sulfate starvation, showing that miRNAs can be induced by environmental stress.

Because miRNAs recognize their regulatory targets through base pairing, computational methods have been invaluable for identifying these targets. The extensive complementarity between plant miRNAs and mRNAs makes systematic target identification easier in plants than in animals (Example 2). A search for targets of 13 Arabidopsis miRNA families predicted 49 unique targets, with a signal-to-noise ratio exceeding 10:1, simply by looking for Arabidopsis messages with three or fewer mismatches (Example 2). Evolutionary conservation of the miRNA:mRNA pairing in rice, as shown above, supports the validity of these predictions. In contrast, metazoan miRNAs only rarely recognize their targets with such extensive complementarity; thus, more sophisticated methods that search for short segments of conserved complementarity to the miRNAs are required to identify metazoan miRNA targets.

The above-identified plant miRNAs have a remarkable propensity to target genes involved in development, particularly those of transcription factors (Example 2). In cases where disruption of plant miRNA regulation has-been reported, striking developmental abnormalities have been observed. Dominant gain-of-function mutations in HD-ZIP transcription factor genes PHABULOSA, PHAVULOTA, and REVOLUTA that destabilize pairing to miR165/miR166 cause loss of adaxial/abaxial polarity in developing leaves. In maize, similar mutations in the HD-ZIP gene ROLLED LEAF1 also cause adaxilization of the abaxial surface of leaves, indicating that the miR165/miR166 family has a conserved role in determining leaf polarity despite the morphological differences between Arabidopsis and maize leaves. Transgenic plants with silent mutations in the miR-JAW complementary sites of TCP transcription factors arrest as seedlings with fused cotyledons and lack shoot apical meristems, while those with mutations in the miR159 complementary site of MYB33 have upwardly curled leaves. Plants deficient in miR172-mediated regulation of APETALA2 have altered patterns of floral organ development. Plants deficient in miR164-mediated regulation of CUP-SHAPED COTYLEDON1 have altered patterns of embryonic, vegetative, and floral development. Finally, silent mutations in the miR168 complementary site of ARGONAUTE1 lead to misregulation of miRNA targets and numerous developmental defects (Example 3).

This example illustrates a computational procedure to identify conserved miRNA genes. Using criteria that retain all 11 of the above-identified miRNA gene families conserved between Arabidopsis thaliana and Oryza sativa, 13 additional families were identified. Molecular evidence verified that at least seven of these newly identified families of candidate miRNAs are authentic, and that at least six out of the seven mediate the cleavage of their mRNA targets. These seven newly identified families were represented by 23 loci. Some targets of the miRNAs, such as F-box proteins and GRL transcription factors, represent genes with demonstrated or probable roles in controlling developmental processes. Other miRNA targets, such as ATP sulfurylases, laccases, and superoxide dismutases, showed that the range of functionalities regulated by miRNAs is quite broad. Furthermore, the expression of miR395, which targets genes involved in sulfate assimilation, is responsive to the sulfate concentration of the growth media, demonstrating that miRNA expression can be modulated by levels of external metabolites.

A PCR based assay was used to detect expression and map the 5′ ends of predicted miRNAs. miRNAs were PCR amplified out of a library of small cDNAs from leaf, flower, and seedling flanked by 5′ and 3′ adaptor oligos (see Example 1). Each PCR reaction used one common primer corresponding the 5′ adaptor oligo and one specific primer antisense to the 3′ portion of the predicted miRNA.

RNA was isolated as previously described. For developmental Northerns, 30 micrograms per lane of total RNA from soil grown Colombia plants were separated by 15% polyacrylamide electrophoresis and blotted to a nylon membrane. For plants grown on media, Columbia plants were grown in long-day conditions on modified MS/agarose media, containing 0.8% Agarose-LE (USBiochem), in which the SO₄²⁻ containing salts of minimal MS media were replaced with their chloride counterparts and the media supplemented with 20 micromolar to 2 mM (NH₄)₂SO₄. RNA was harvested from 2-week old plants; For miRNA Northerns, 40 micrograms per lane was used in Northern blots as above. For miR393, miR394, miR396a and miR398b, end-labeled antisense DNA probes were used. For miR395a, miR397b, and miR399b, higher specific activity Starfire (Integrated DNA technologies) probes were used. MicroRNA Northerns were hybridized and washed using established techniques. For mRNA Northerns, 10 micrograms per lane were separated by agarose electrophoresis and blotted using known techniques. Probes to exon 1 of APS 1 were made using the Megaprime DNA labeling system (Amersham).

5′-RACE was performed on poly(A)-selected RNA from Columbia inflorescences and rosette leaves using the GeneRacer Kit (Invitrogen), except that nested PCR was done for each gene, with each round of PCR using one gene-specific primer and the GeneRacer 5′ Nested Primer. For each gene gene-specific primers were designed that were 180 bp to 450 bp away from the predicted miRNA binding site. PCR reactions were separated by, agarose gel electrophoresis, and distinct bands of the appropriate size for miRNA-mediated cleavage were purified (excised gel slices corresponded to a size range of approximately 100 base pairs), cloned, and sequenced.

The computational approach used in this example to identify plant miRNAs was based upon six characteristics that describe previously known plant miRNAs. 1) The base pairing of the mature miRNA to its miRNA*within the hairpin precursors was relatively consistent. In contrast, both the size of the foldback and the extent of base pairing outside of the immediate vicinity of the miRNA were highly variable among the hairpins of plant miRNAs, even among those of miRNAs from the same gene family. 2) The majority of known Arabidopsis miRNAs have identifiable homologs in the Oryza sativa genome, in which the predicted mature Oryza miRNAs had 0 to 2 base substitutions relative to their Arabidopsis homologs. 3) The secondary structures of known miRNA hairpins were accurately predicted by RNAfold when given a sequence sufficiently long to contain both the miRNA and the miRNA*. 4) The sequences of the Arabidopsis and Oryza hairpins were generally more conserved in the miRNA and miRNA*than in the segment joining the miRNA and miRNA*. 5) All matches to known miRNAs in the Arabidopsis genome, with the exception of those antisense to coding regions, had potential miRNA-like hairpins and were thus properly annotated as miRNA genes. 6) Most known Arabidopsis miRNAs were highly complementary to target mRNAs, and this complementarity was conserved to Oryza.

FIG. 20A illustrates an outline of the computational approach used to identify conserved plant miRNAs used in this example. In Steps 1-8, the sensitivity is reported as the fraction of miRNA loci retained with perfect matches to previously identified miRNAs (refset1). In Step 9, this fraction extends to imperfect matches to previously identified miRNAs. In the later steps, the total numbers of predicted miRNA loci are also reported.

As the first step to identifying miRNAs in the genomes of Arabidopsis thaliana and Oryza sativa, only those genomic portions were considered contained in imperfect inverted repeats as defined by EINVERTED (FIG. 20A, Step 1). Within these 133,864 Arabidopsis and 410,167 Oryza inverted repeats were 73 of 86 reference set loci corresponding to the 24 established miRNAs (refset 1, FIG. 21; see also Example 1). Secondary structures for the inverted repeats were predicted with RNAfold, and all 20-mers within the inverted repeats were checked against MIRcheck, an algorithm written to, identify 20-mers with the potential to encode miRNAs (FIG. 20A, Step 2). MIRcheck takes as input a) the sequence of a putative miRNA hairpin, b) a secondary structure of the putative hairpin, and c) a 20-mer sequence within the hairpin to be considered as a potential miRNA. MIRcheck takes into account the total number of unpaired nucleotides (no more than 4 in the putative miRNA), the number of bulged or asymmetrically unpaired nucleotides (no more than 1 in the putative miRNA), the number of consecutive unpaired nucleotides (no more than 2 in the putative miRNA) and the length of the hairpin (at least 60 nucleotides inclusive of the putative miRNA and miRNA*). In contrast to the algorithms designed to identify metazoan miRNAs, MIRcheck has no requirements pertaining to the pattern or extent of base pairing in other parts of the predicted secondary structure. Even though these parameters were chosen to be relatively stringent, only 7 of the 73 remaining Arabidopsis and Oryza refset1 loci were lost at this step.

After removal of 20-mers that overlap with repetitive elements, or which have highly biased sequence compositions, 389,648 Arabidopsis 20-mers (AtSet1) and 1,721,759 Oryza 20-mers (OsSet1) had at least 1 locus that passed MIRcheck. Patscan was then used to identify 20-mers in AtSet1 that matched at least one 20-mer in OsSet1 with 0 to 2 base substitutions, considering only 20-mers on the same arm of their putative hairpins (FIG. 20A, Step 3). 3,851 Arabidopsis 20-mers had at least 1 Oryza match (AtSet2), and 5,438 Oryza 20mers were matched at least once (OsSet2).

For the known plant miRNAs, RNAfold predicted a secondary structure in which the miRNA is paired to the miRNA*, provided that the flanking sequence is sufficiently long to contain the miRNA* (see Example 1). The presence of additional flanking sequence did not interfere with the prediction of an miRNA-like secondary structure. This robustly predicted folding was observed for all of the loci of each cloned miRNA, even though they had widely divergent flanking sequences. While recognizing that the predicted folds were unlikely to be correct in all their details, it is reasonable to assume that the overall robustness of the predicted folding reflects an evolutionary optimization for defined folding in the plant. To eliminate candidates that did not fold as robustly as the previously known miRNAs, the AtSet2 and OsSet2 20 -mers were required to pass MIRcheck a second time after being computationally folded in the context of sequences flanking the hairpin. Patscan was used to find all matches of AtSet2 and OsSet2 to their respective genomes, RNAfold was used to predict the secondary structure of each match in the context of a 500 nucleotide genomic sequence centered on the 20-mer, and each match was evaluated by MIRcheck (FIG. 20A, step 4). 2,588 Arabidopsis 20-mers (AtSet3) and 3,083 Oryza 20-mers (OsSet3) had at least one locus that passed MIRcheck. Because EINVERTED misses some hairpins and because this second MIRcheck evaluation used more relaxed cutoffs (up to 6 unpaired nucleotides each in the putative miRNA and miRNA*), this step also recovered paralogs that were missed in steps 1 or 2:

The genomic matches to known Arabidopsis miRNAs were all either in hairpins or antisense to coding regions. To ensure that computationally identified miRNAs met this criterion, Arabidopsis 20-mers were removed from the analysis if less than 50% of intergenic matches passed MIRcheck, or if more than 50% of genomic matches overlapped with repetitive sequence elements (FIG. 20A, Step 5), resulting in 2,506 20-mers (AtSet4). Because gene annotation in Oryza was poor, the matches could not be reliably defined as genic or intergenic. The 2,780 Oryza 20-mers that had at least 1 locus that passed MIRcheck and had no more than 50% of genomic matches in repetitive sequence elements were included in OsSet4.

The next step in this analysis was to identify pairs of Arabidopsis and Oryza hairpins that had miRNA-like patterns of sequence conservation (FIG. 1a, step 6). MicroRNA precursors are generally most conserved in the miRNA:mRNA*portion of the hairpin. In this procedure, homologous pairs were retained for which both the miRNA and miRNA*20-mers were more conserved than any 20-mer from the loop regions. Doing pairwise comparisons of the hairpins of AtSet4 against those of OsSet4 resulted in 1,145 20-mers (AtSet5) with at least 1 acceptable Oryza homolog.

AtSet5 was mapped to the Arabidopsis genome, and overlapping 20-mers were joined together to form 379 sequences with miRNA encoding potential. A single miRNA gene could be represented by up to four of these potential miRNA sequences, representing the miRNA, the miRNA*, the antisense miRNA, and the antisense miRNA*. After accounting for multiple potential miRNAs mapping to a single locus, the 379 potential miRNAs represented 228 potential miRNA loci. These 228 loci were grouped into 118 families of potential miRNA loci based on sequence similarity as determined by blastn. Many of these newly identified miRNA candidates had patterns of secondary structure conservation resembling those of previously known plant miRNAs (e.g., FIG. 20B and 20C). For many of the miRNA loci corresponding to previously reported miRNAs, the computationally identified sequences extended 1-9 nucleotides on either side of the cloned miRNAs, although in a few cases the actual miRNA overlapped with but extended beyond the predicted sequence.

FIGS. 20B and 20C illustrate hairpin secondary structures of two newly identified miRNA families, 393 (FIG. 20B) and 394 (FIG. 20C) that target mRNAs of F-box proteins. Nucleotide sequences 50 comprise the sequence of the most common mature miRNA as deduced from PCR validation and Northern hybridization. Nucleotide sequences 51 indicate additional portions of the hairpins predicted to have miRNA-encoding potential after identification of conserved 20-mers in miRNA-like hairpins (FIG. 20A, Step 6), but before identification of conserved complementarity to mRNAs or experimental evaluation. For all three MIR393 loci, sequences antisense to the validated miRNA were also identified as potentially miRNA-encoding, but the miRNA*segments were not.

The procedure in this example allows for gaps and mismatches in the mRNA:miRNA duplex but requires that the miRNA complementarity be conserved between homologous Arabidopsis and Oryza mRNAs. Each miRNA complementary site was scored, with perfect matches given a score of 0, and points were added for each G:U wobble (0.5 points), each non-G:U mismatch (1 point) and each bulged nucleotide in the miRNA or target strand (2 points). To allow the same cutoffs to be applied more evenly to miRNAs of different lengths and to avoid penalizing mismatches at the ends of longer miRNAs, those miRNAs that were longer than 20 nucleotides were broken into overlapping 20-mers, with the mRNA:miRNA pair receiving the score of the most favorable 20-mer.

This scoring was tested using a set of 10 unrelated miRNAs that were highly conserved (0 to 1 substitutions) between Arabidopsis and Oryza (refset2, FIG. 21). As a control, 5 cohorts of permuted miRNAs were generated, in which each permuted miRNA has the same dinucleotide composition as the corresponding miRNA in refset2. For all 20-mers from the sets of real and permuted miRNAs complementary sites in Arabidopsis and Oryza mRNAs were searched. Compared to their shuffled cohorts, the real miRNAs had many more complementary Arabidopsis mRNAs with scores≦2 (FIG. 22A), which was in agreement with Example 2. Filtering the miRNA-complementary mRNAs to include only those conserved to Oryza showed that nearly all the complementary sites to authentic miRNAs with scores of ≦2 are conserved (FIG. 22B).

In FIG. 22A, the number of mRNAs with each of the indicated scores is graphed (solid bars). Complementary sites were found and scored in the same manner for 5 cohorts of permuted miRNAs with the same dinucleotide composition as the authentic miRNAs (open bars, average number of complementary mRNAs per cohort; error bars, 2 standard deviations). FIG. 22B illustrates mRNAs complementary to 10 miRNAs as were found as in FIG. 22A, with the additional requirement that at least one homologous Oryza mRNA be complementary to the same miRNA (solid bars). Each conserved miRNA complementary site was counted as having the either the Arabidopsis or Oryza score, whichever is higher (i.e. less complementary). Messenger RNAs-with conserved complementarity to cohorts of dinucleotide shuffled miRNAs were found in the same manner (open bars, average number of complementary mRNAs; error bars, 2 standard deviations).

For the permuted miRNAs, requiring conservation reduced to nearly zero the number of complementary sites with scores of 2.0 to 3.5, whereas for the authentic miRNAs a small but significant number of sites scoring in this range were conserved (FIG. 22B). Thus, adding a requirement for conservation raised the threshold at which spurious matches were found, thereby enabling confident prediction of targets that were less extensively paired to the miRNAs, in some cases forming Watson-Crick pairs to only 15 of 20 miRNA nucleotides.

Each of the conserved miRNAs had at least one predicted target with score≦3.0, suggesting that the possession of predicted targets could be a criterion for screening the newly identified miRNA candidates. For each 20-mer in AtSet5 and OsSet5, miRNA complementary sites were found and scored (FIG. 20A, Step 7). As would be expected even for permuted sequences, nearly all of the AtSet4 20-mers (1,124 out of 1,145) had a complementary score of ≦3.0 to at least 1 Arabidopsis mRNA. Of these, 278 20-mers (AtSet6) had at least one homologous Oryza 20-mer with complementarity to a homologous Oryza mRNA. AtSet6 represented 24 families of potential miRNAs, which account for 100 potential miRNA loci. Eleven of these families, represented by 60 loci (including 41 refset1 loci), corresponded to all previously known miRNA families with identifiable Oryza homologs, showing that this method also identified most of the previously unknown families that have extensive conserved complementarity in Oryza.

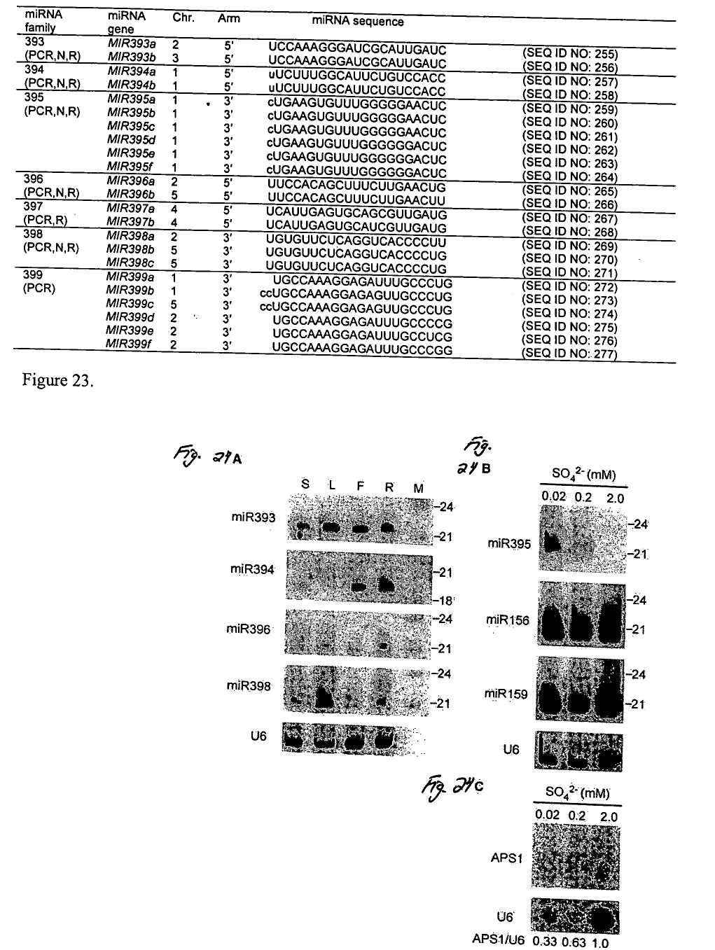

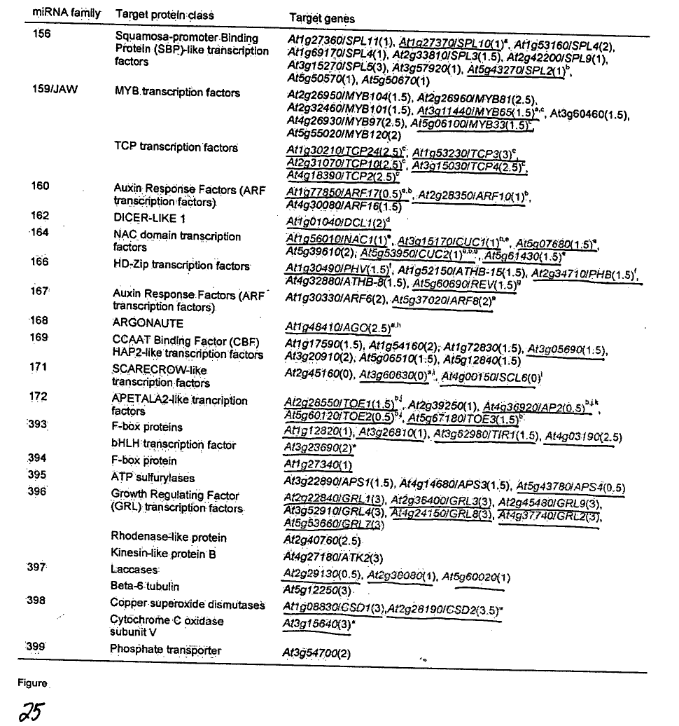

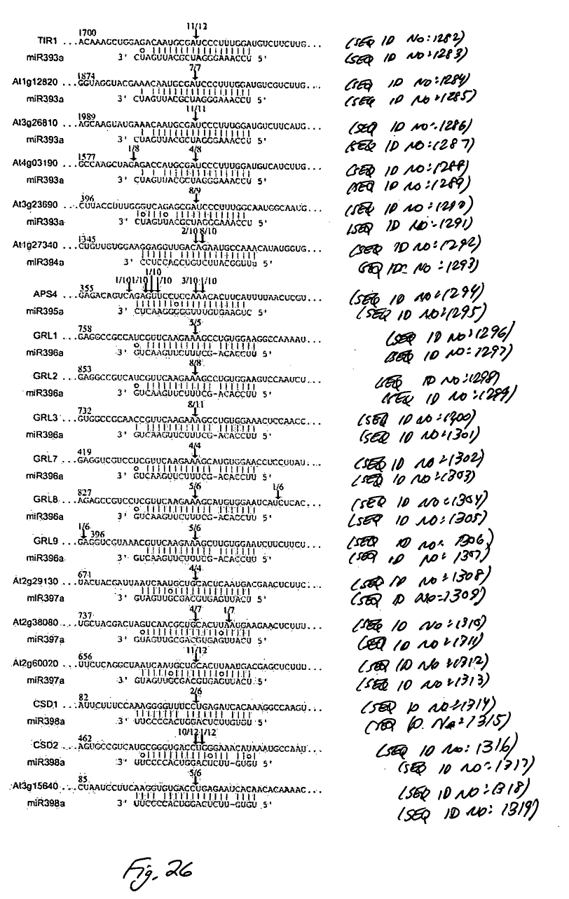

This computational screen identified 13 previously unreported families of conserved miRNA candidates with conserved complementarity to mRNAs; To determine which of these putative miRNAs were expressed, a PCR based assay was used to search for the predicted miRNAs in a library of small cDNAs. In addition to verifying the expression of the miRNAs, this assay mapped the 5′ ends of the miRNAs (FIG. 23). In FIG. 23, the miRNA families are listed with a summary of the experimental validation (PCR, PCR validation of miRNA; N, Northern blot of miRNA; R, 5′-RACE of target mRNA). The chromosome of each locus is also indicated (Chr.), as is the arm of the predicted stem-loop that contains the miRNA (arm). 5′ ends of miRNAs were determined from PCR of small cDNAs, and lengths of miRNAs were inferred from mobility on Northern blots. For miRNAs not detected on Northern blots (families 397 and 399), lengths of 21 nucleotide were assumed. For miRNA families for which multiple 5′ ends were detected by PCR, nucleotides present in some but not all clones are listed in lower case.

Each PCR reaction used one common primer corresponding to the adaptor oligo attached to the 5′ end of all members of the library and one primer specific the 3′ portion of the predicted miRNA. For seven miRNA families, PCR reactions resulted in products in which the specific primer was extended by at least 3 nucleotides that matched the predicted miRNA sequence. In sum, the seven newly identified miRNA families comprised 23 genomic loci in Arabidopsis (FIG. 23). All clones for families 393, 396, 397, and 398 had the same 5′ end, while for families 394, 395, and 399 miRNAs were detected with differing 5′ ends that could result from inconsistent processing of precursors transcripts from a single locus, or from differential processing of precursors from different loci. Several of these miRNA families include loci that would encode distinct but highly similar miRNAs (FIG. 23). Because the PCR primers overlapped with the residues that differ, it is not possible to know which variants were detected.

Six families of putative miRNAs passed all computational checks but were not validated by the PCR assay. Five of these families had a single locus in Arabidopsis, whereas the sixth had 14 Arabidopsis loci and 52 Oryza loci and likely represented a repetitive element not identified by RepeatMasker.

The expression of newly identified miRNAs was also tested by Northern blot analysis. Hybridization probes were designed for representative members of the 7 miRNA families detected by the PCR assay. Probes complementary to miR393, miR394, miR396a, miR398b detected 20-21 nt RNAs in samples from wild-type, soil-grown Columbia plants (FIG. 24A), whereas probes complementary to miR395a, miR397b, and miR399b did not detect expressed small RNAs in these samples. These miRNAs that are difficult to detect on a Northern blot are likely to be expressed only at low levels or only in a subset of tissues or growth conditions.

FIG. 24A shows the total RNA (30 micrograms) from seedlings (S), rosette leaves (L), flowers (F), and roots (R) that were analyzed on a Northern blot, successively using radio-labeled DNA probes complementary to newly identified miRNAs. The lengths of 5′-phosphorulated radio-labeled RNA size markers (M) are as indicated. As a loading control, he blot was probed for the U6 mRNA. FIG. 24B shows that miR395 was induced with low sulfate. Total RNA (40 microgram) from 2-week-old Columbia plants grown on modified MS media containing the indicated concentrations of SO₄²⁻ were analyzed by Northern blot, probing for the indicated miRNAs as in FIG. 24A. FIG. 24C illustrates APS1 mRNA decreases in low sulfate. Total RNA (10 microgram) from 2-week-old plants grown on modified MS media containing the indicated concentrations of SO₄⁻² were analyzed by Northern hybridization using randomly primed body-labeled DNA probes corresponding to exon 1 of the APS1 mRNA. Normalized ratios of APS1 mRNA to U6 splicosomal RNA are indicated.

Because miR395 was complementary to mRNAs of ATP sulfurylase (APS) proteins (FIG. 25), and because the expression levels of numerous sulfate metabolizing genes were responsive to sulfate levels, the expression of miR395 may depend on cellular sulfate levels. To test this, RNA samples were probed from plants grown in modified MS media containing various amount of sulfate. As seen for plants grown in soil, miR395 was not detected in the samples from plants grown in 2 mM SO₄⁻². However, miR39.5 was readily detected in the samples grown in very low sulfate (FIG. 23B, 0.2 or 0.02 mM SO₄⁻²). Induction of miR395 by low external sulfate concentrations caused changes of greater than 100 fold. APS1 expression was examined to determine changes in the conditions that induced miR395, and it was found that its expression decreased when miR395 increased, as would be expected if APS1 was a cleavage target of miR395 (FIG. 23C).

MicroRNAs can direct the cleavage of their mRNA targets when these messages have extensive complementarity to the miRNAs. This miRNA-directed cleavage can be detected by sing a modified form of 5′-RACE (rapid amplification of cDNA ends) because the 3′ product f the cleavage had these diagnostic properties: 1) a 5′ terminal phosphate, making it a suitable substrate for ligation to an RNA adaptor using T4 RNA ligase, and 2) a 5′ terminus that maps precisely to the nucleotide that pairs with the tenth nucleotide of the miRNA. To examine whether any of the newly identified miRNAs can direct cleavage of their predicted targets in vivo, RNA was isolated from vegetative and floral tissues and the 5′-RACE procedure was performed using primers specific to the predicted targets. For 19 predicted targets the 5′-RACE PCR yielded a distinct band of the predicted size on an agarose gel, which was isolated, cloned and sequenced. In these 19 cases, the most common 5′ end of the mRNA fragment mapped to the nucleotide that pairs to the tenth nucleotide of one of the miRNAs validated by PCR (FIG. 26), indicating cleavage at sites precisely analogous to those seen for other miRNA targets.

In FIG. 26, each top strand depicts a miRNA complementary site, and each bottom strand depicts the miRNA. Watson-Crick pairing (vertical dashes) and G:U wobble pairing (circles) are indicated. Arrows indicate the 5′ termini of mRNA fragments isolated from plants, as identified by cloned 5′-RACE products, with the frequency of clones shown. Only cloned sequences that matched the correct gene and had 5′ ends within a 100 nucleotide window centered on the miRNA complementary site are counted. The miRNA sequence shown corresponds to the most common miRNA suggested by miRNA PCR validation (FIG. 23). For miR394, the 5′ end of a less common variant (1 out of 4 PCR clones) is indicated in lower case and corresponds to the most commonly cloned cleavage product. These observations also corroborate the 5′ ends of the miRNAs as mapped by PCR (FIG. 23).

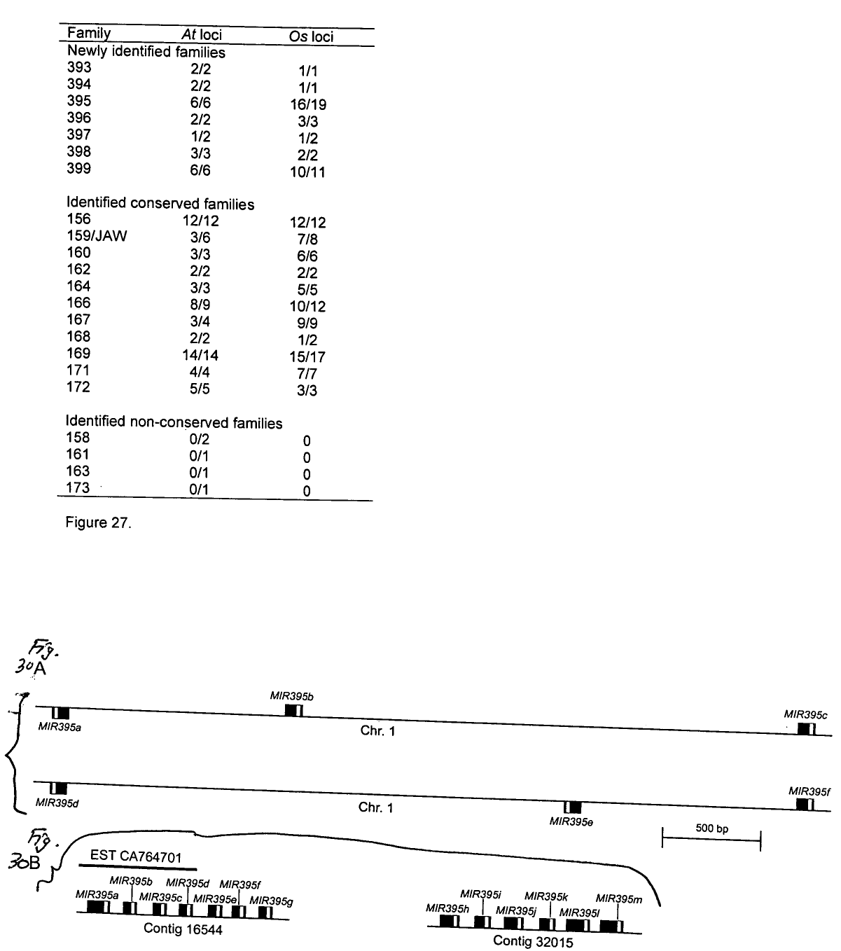

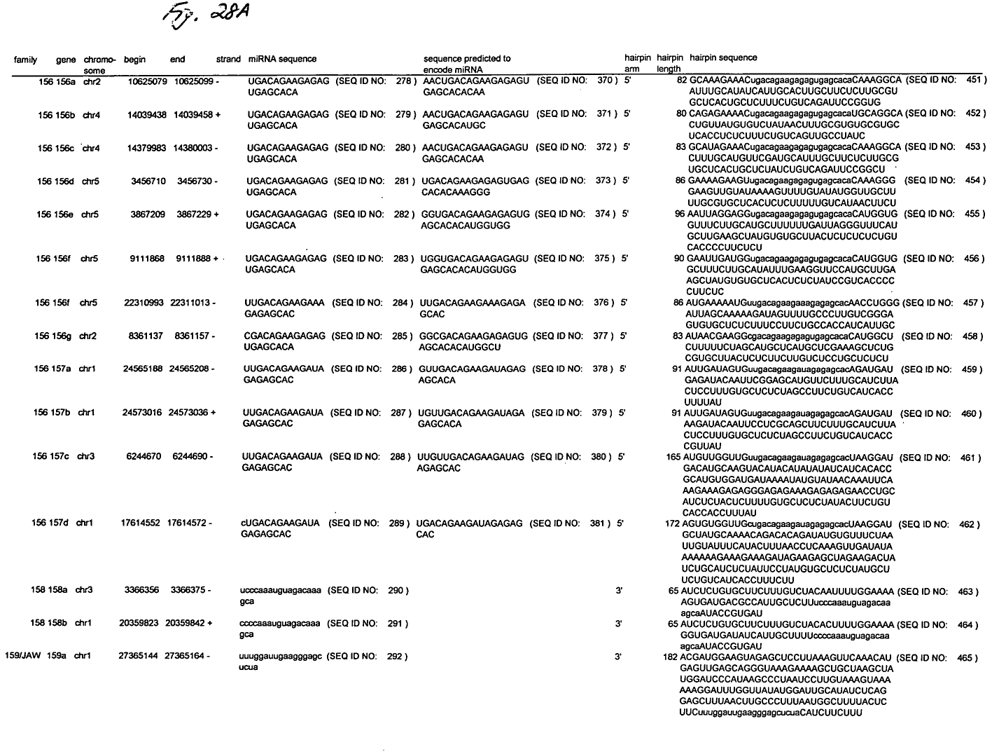

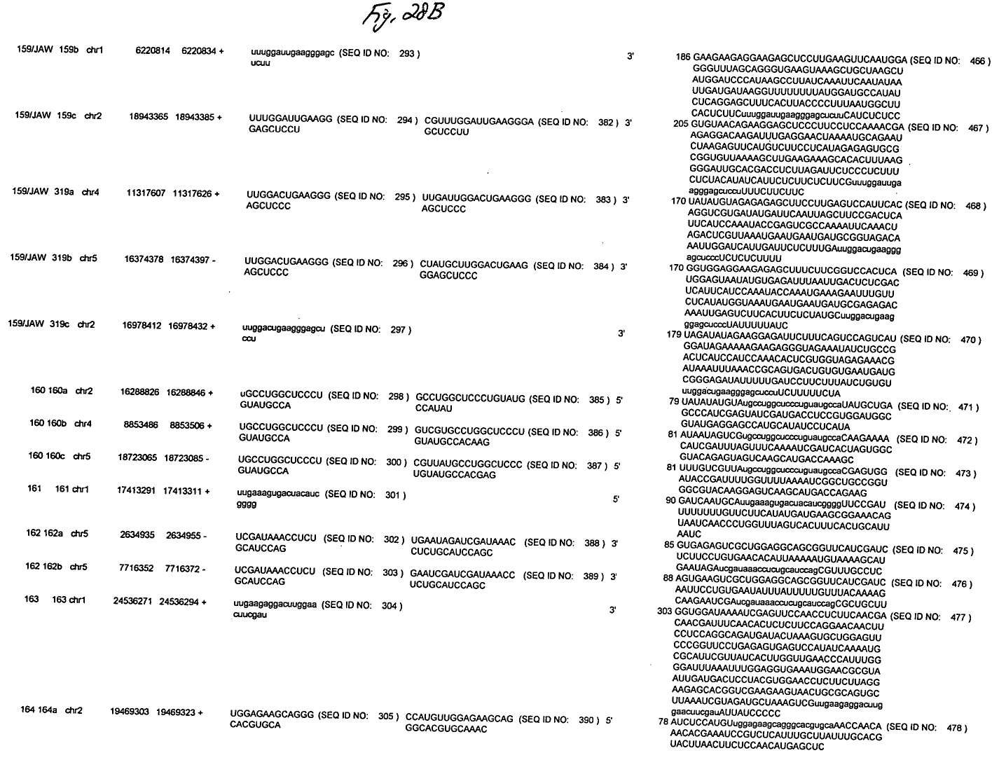

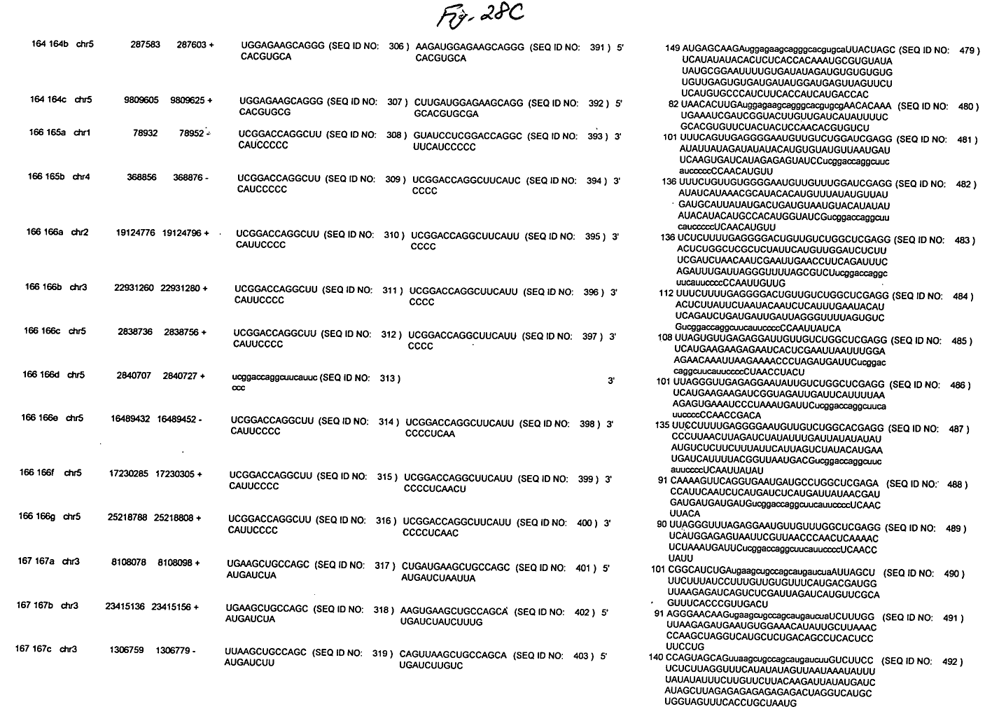

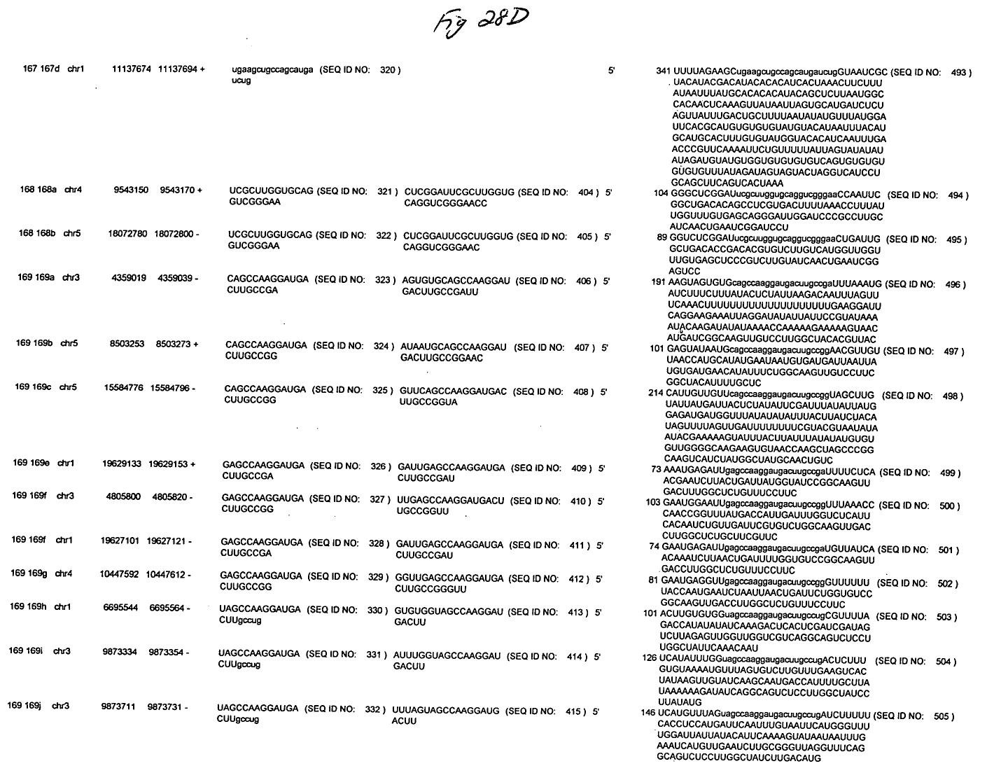

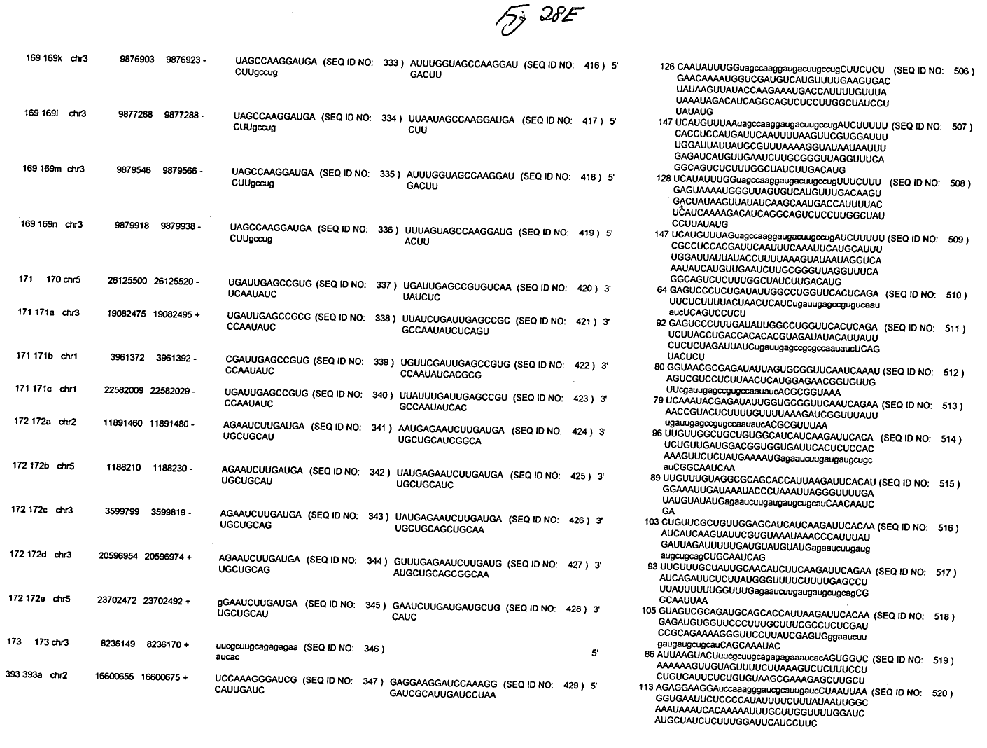

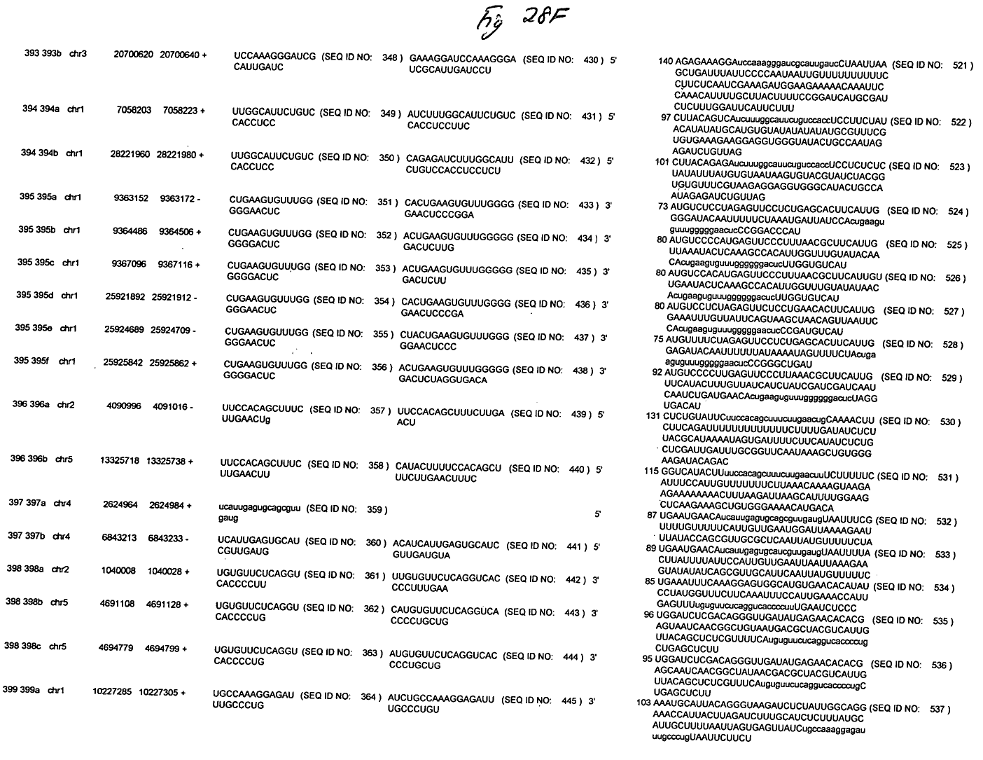

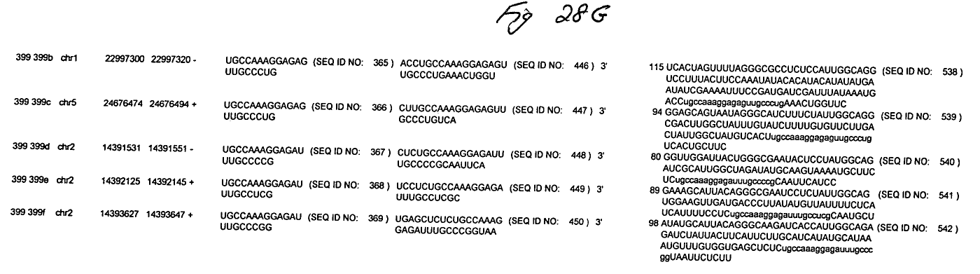

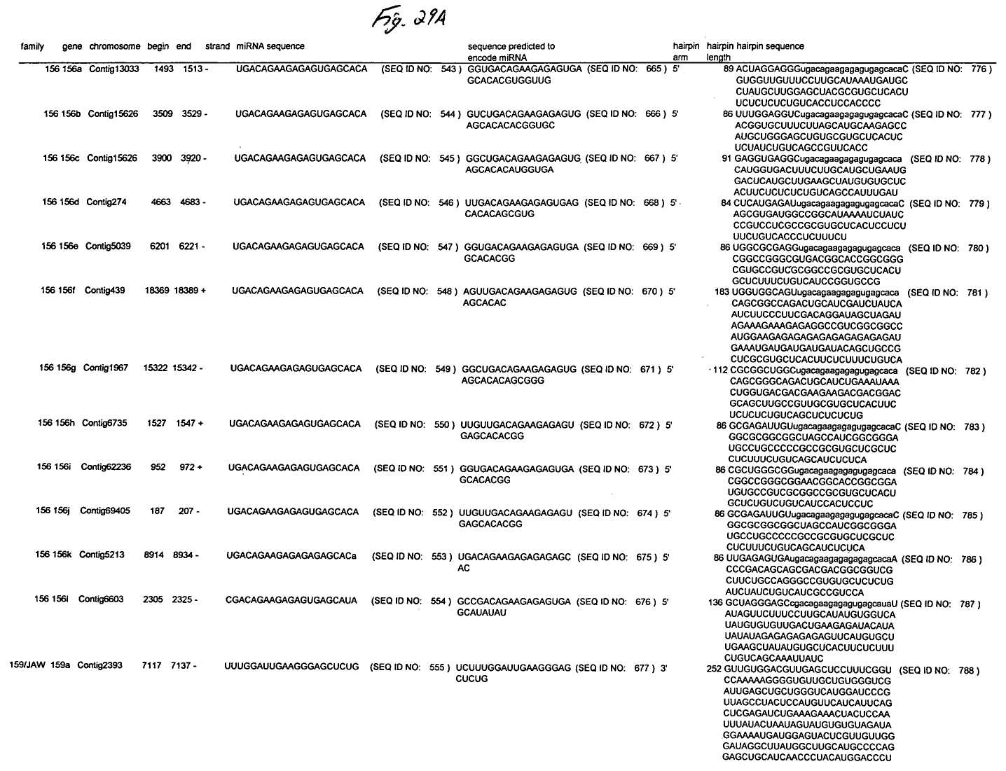

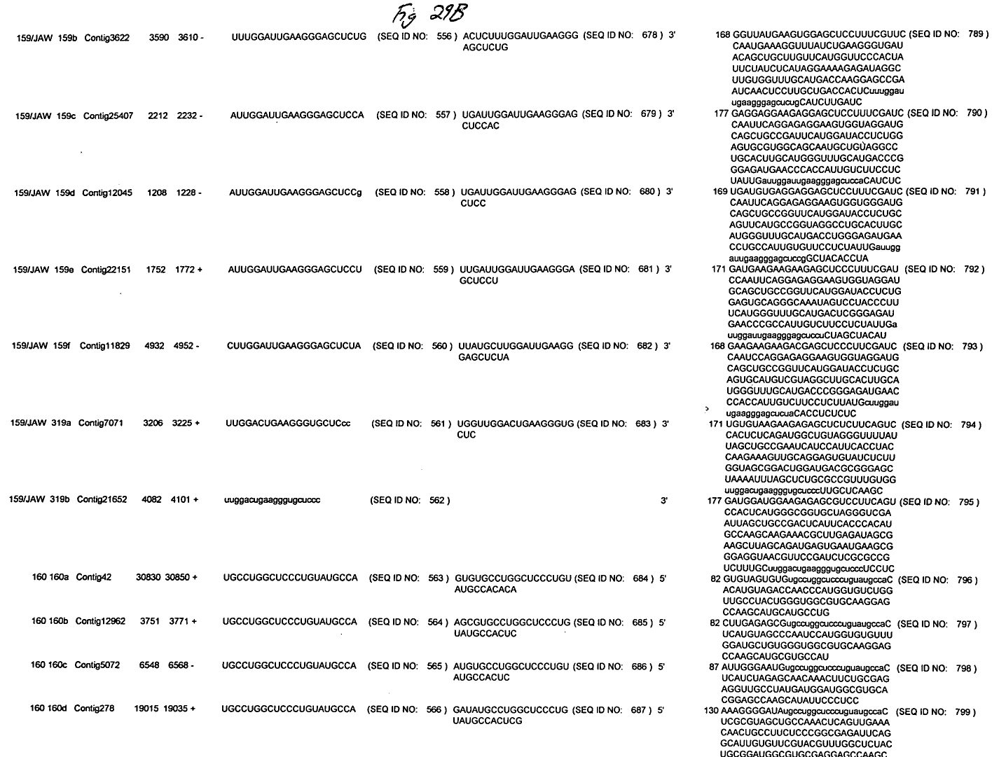

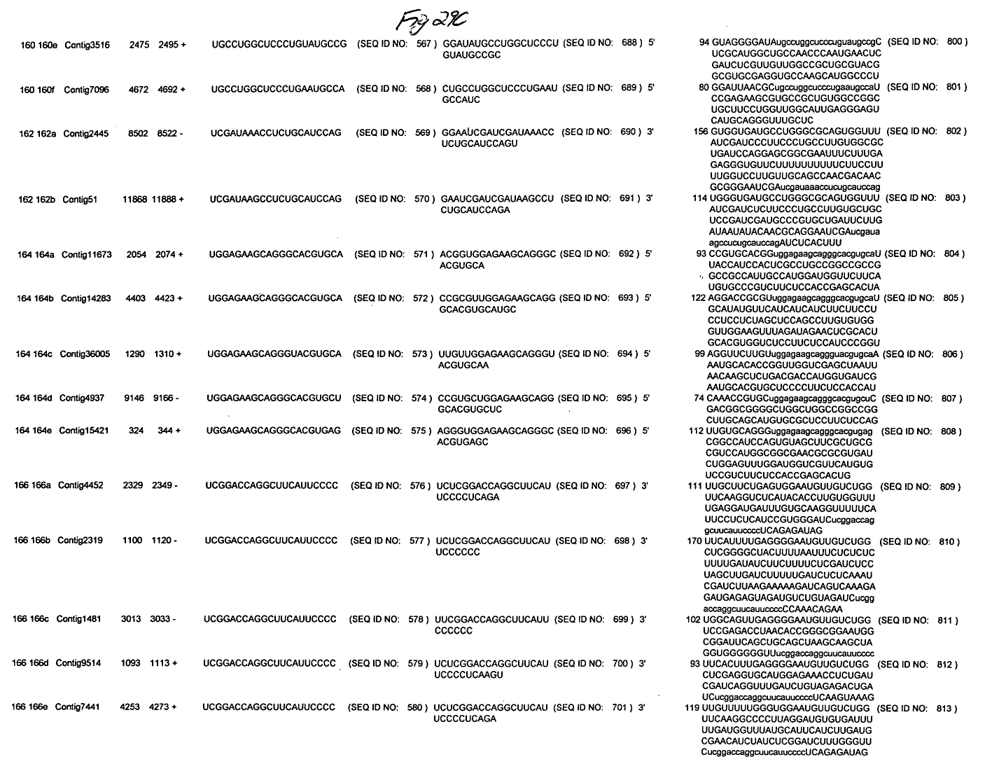

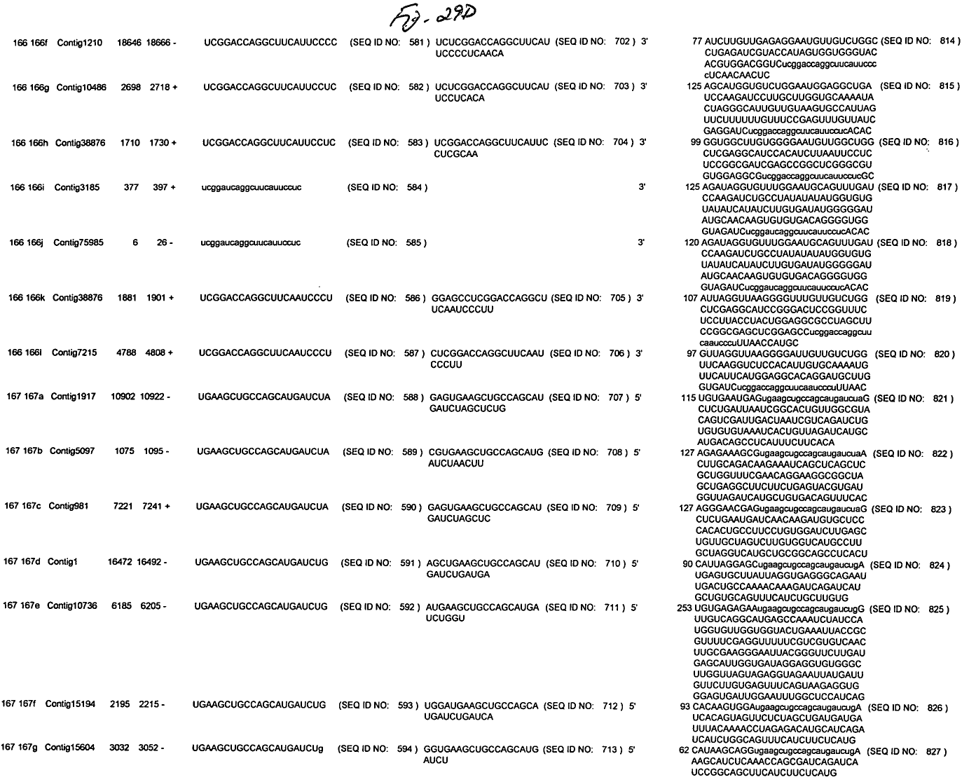

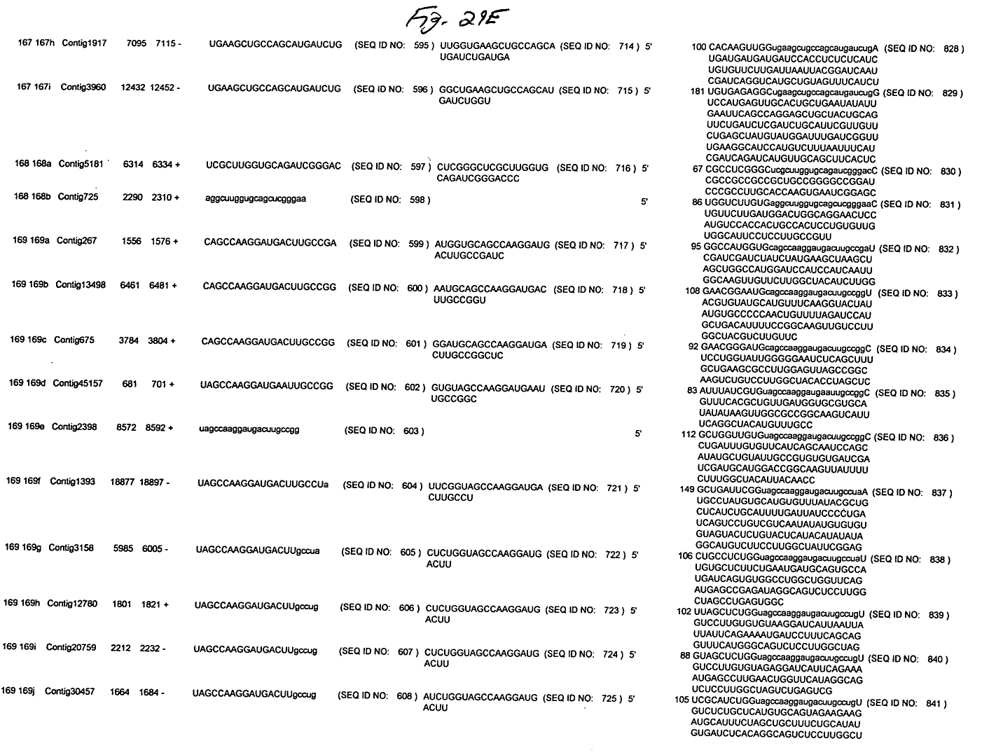

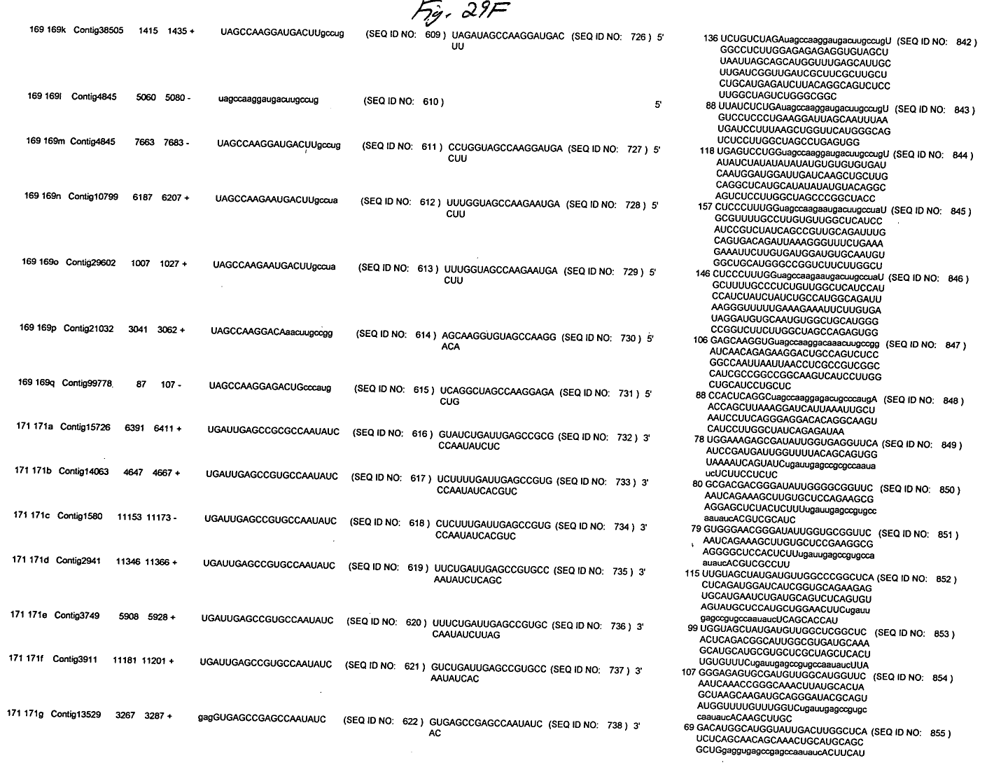

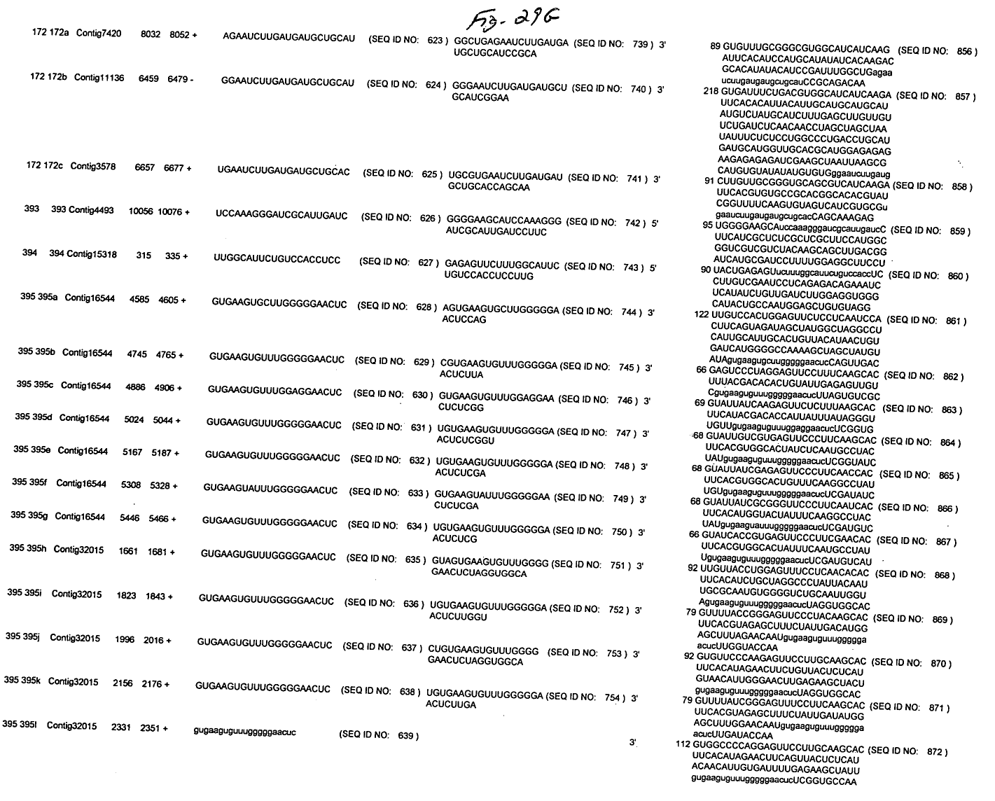

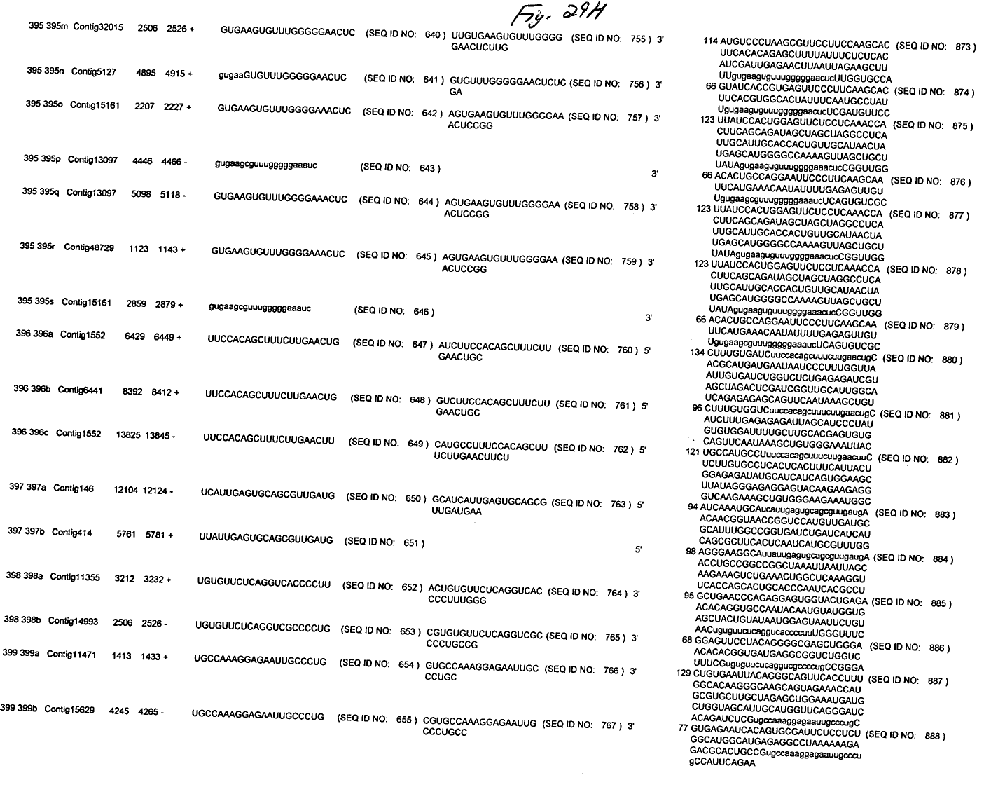

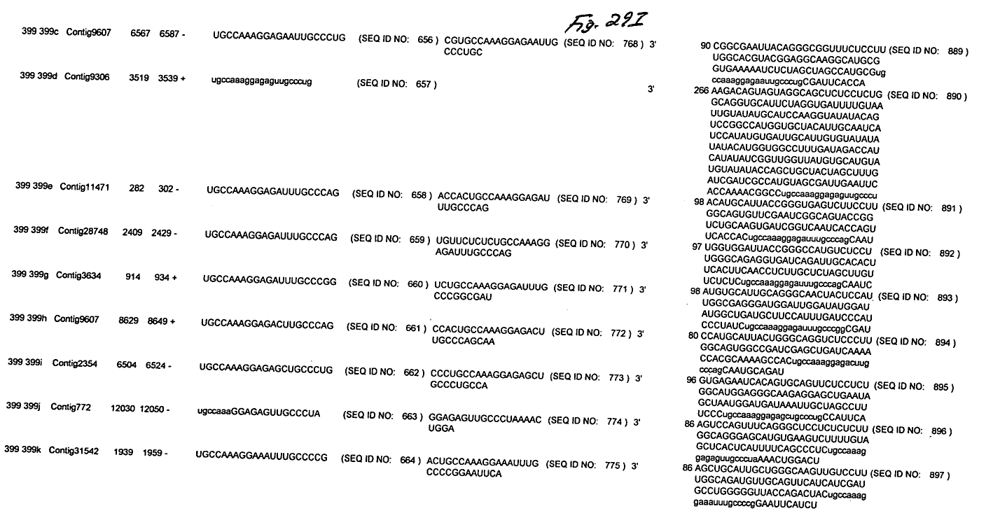

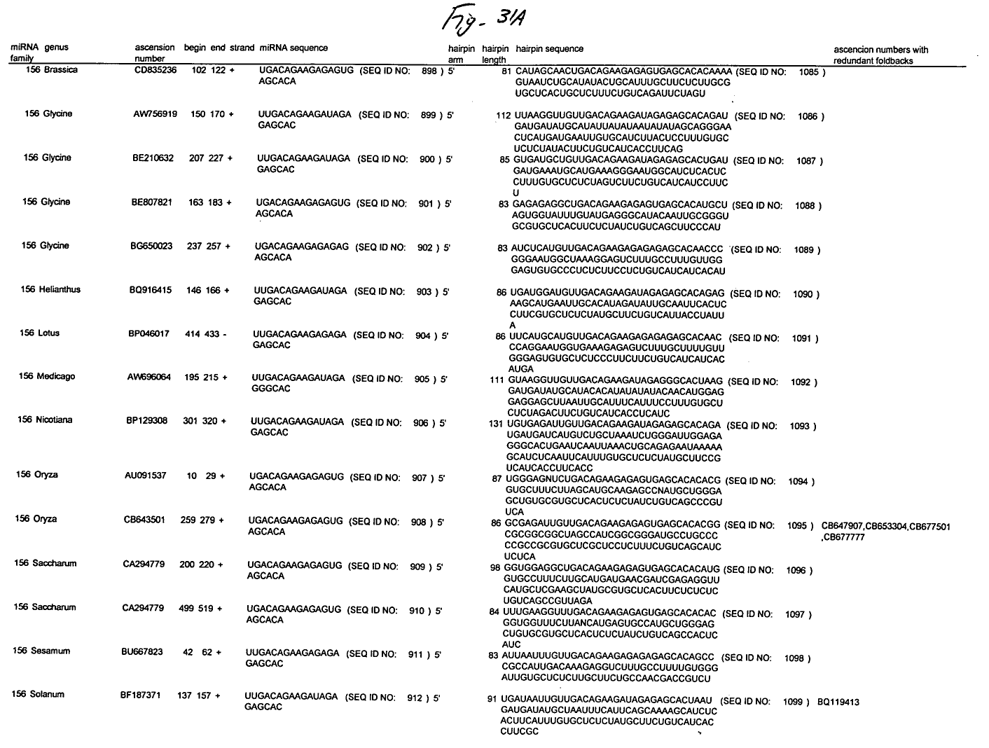

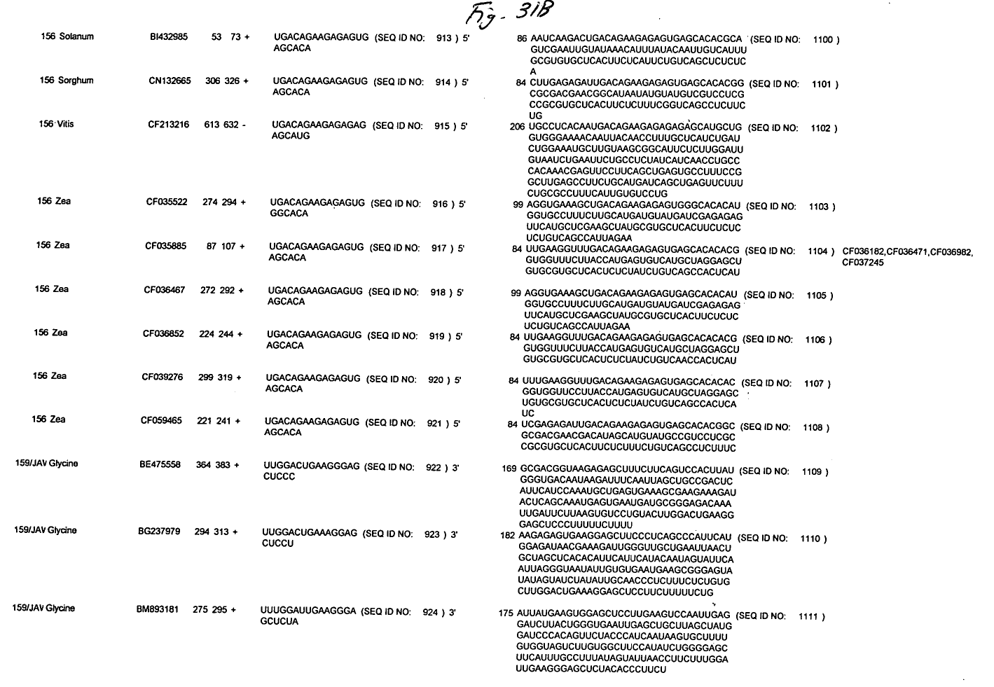

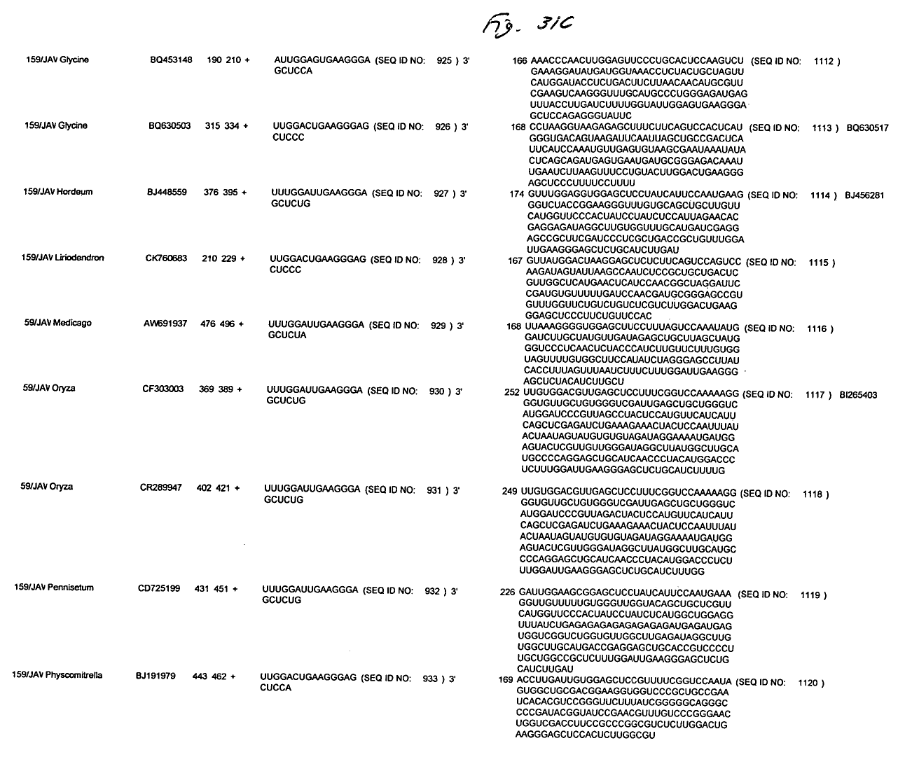

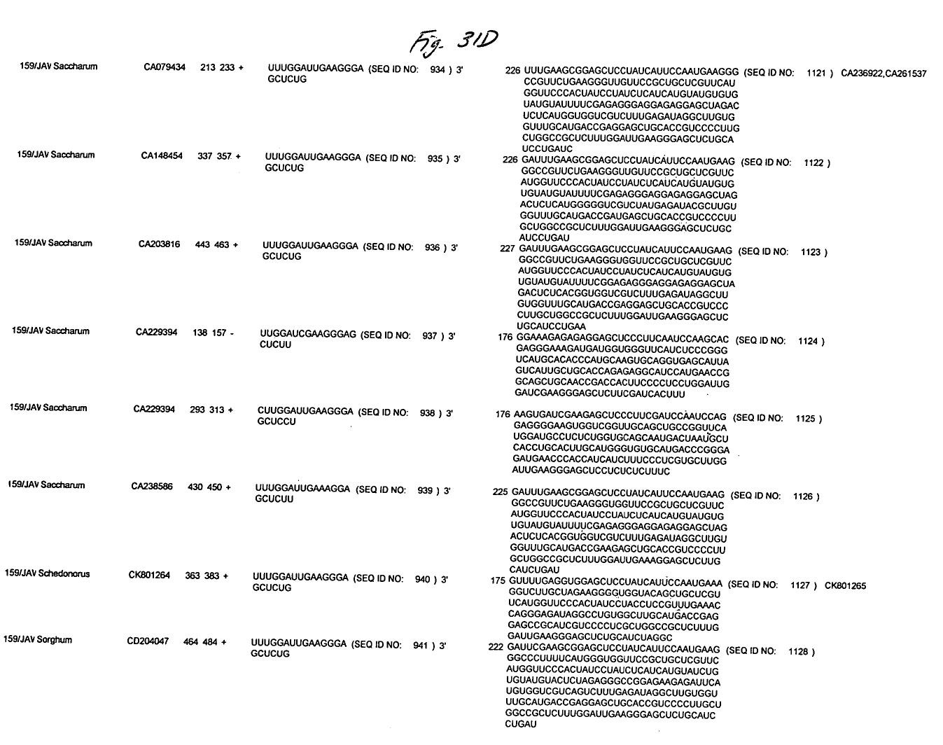

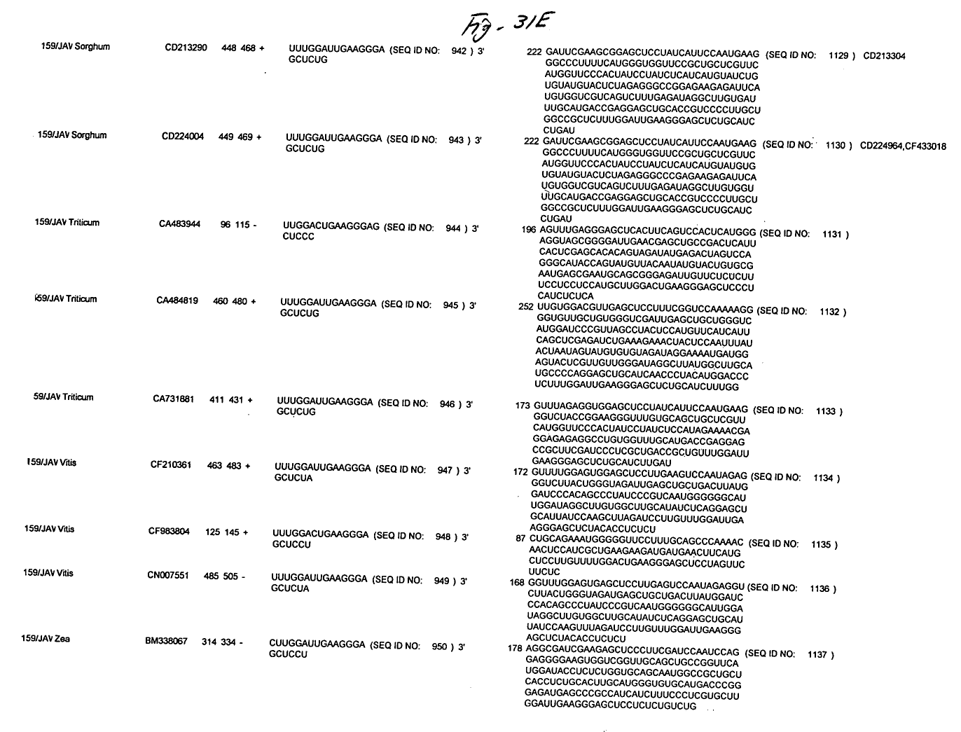

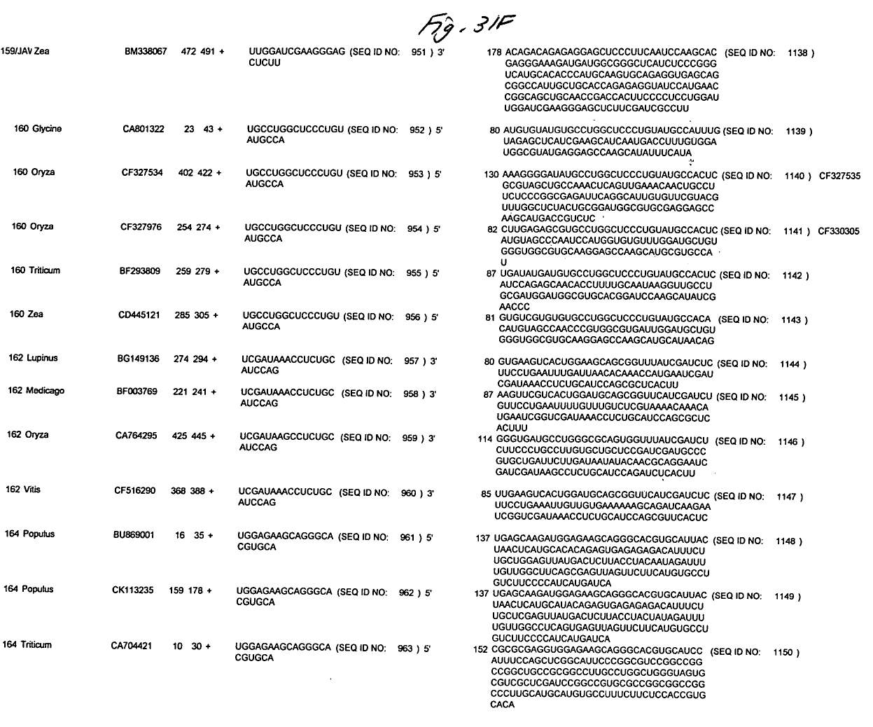

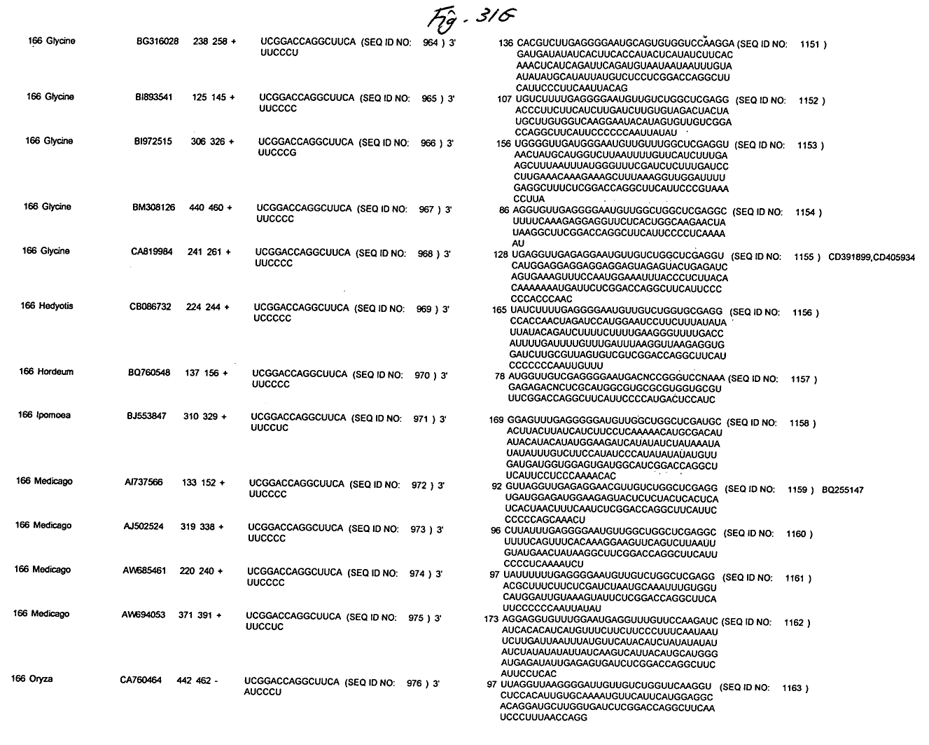

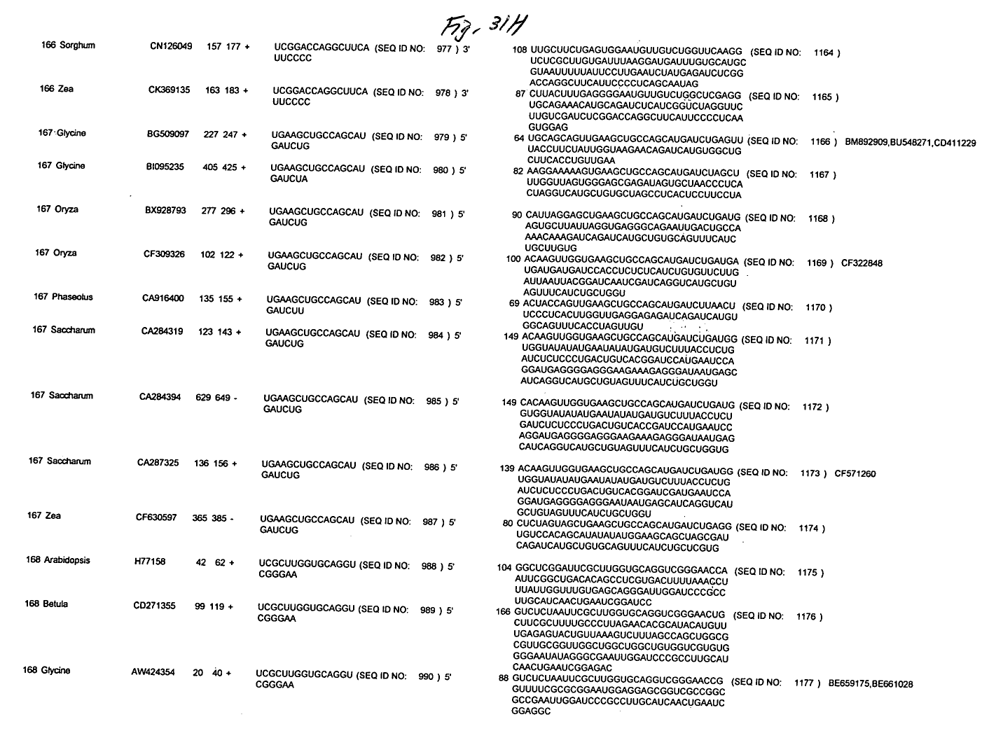

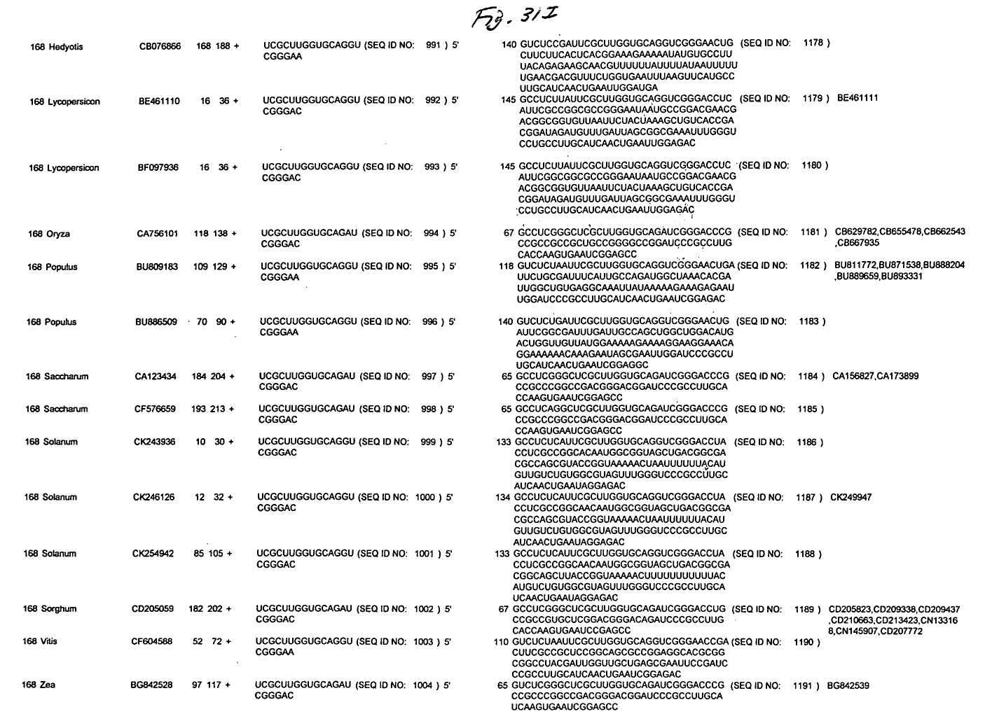

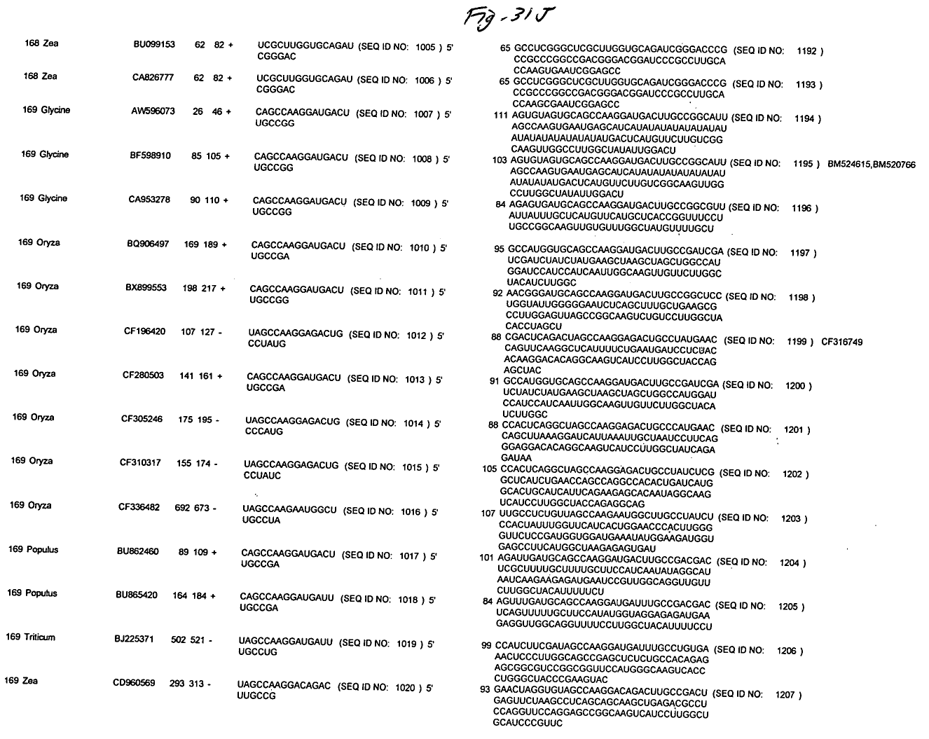

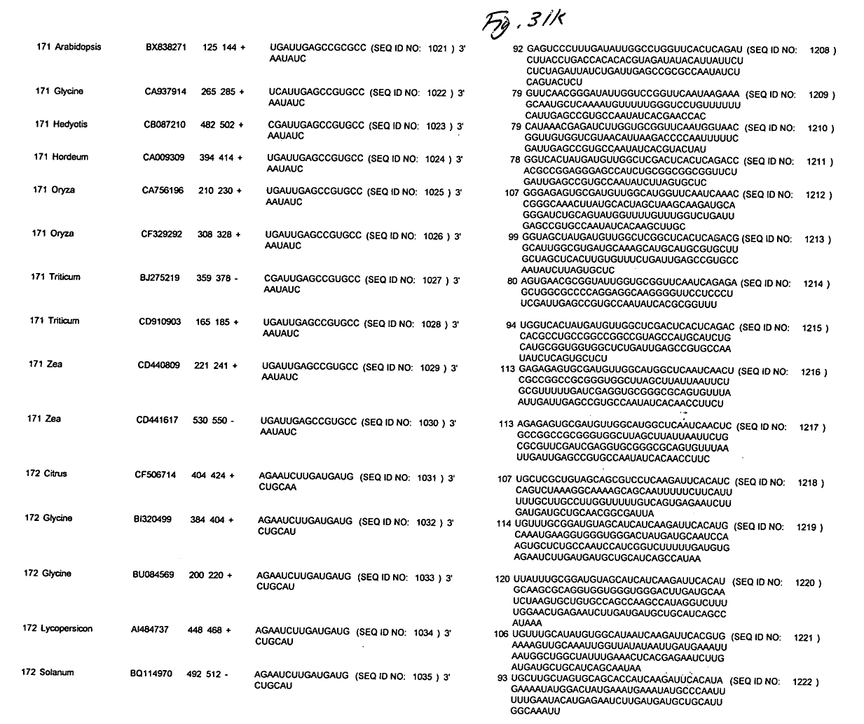

This approach identified 81 miRNA loci from 18 miRNA families (FIG. 27 and 28). FIG. 27 illustrates a number of miRNA families. The number of loci found by de novo computational prediction (FIG. 20A) is shown (numerator) as fraction of total found by searching for near paralogs to miRNAs with verified expression (denominator). Additional details regarding the miRNA loci are reported in FIGS. 28 and 29 (Arabidopsis and Oryza loci, respectively). In FIGS. 28 and 29, the Arabdiopsis and Oryza miRNAs were identified by cloning, computational prediction, and homology to validated miRNAs. The sequences of the mature miRNAs are shown, as are the portions of each locus computationally predicted to have miRNA encoding potential. Nucleotides in the mature miRNAs outside of the computationally predicted region are in lowercase. For miRNA families that have not been cloned, the 5′ end of the sequence was determined by PCR of the miRNA and the length is inferred from mobility on a Northern blot. For miRNA loci that are related to a cloned miRNA, the 5′ and 3′ ends are inferred from the ends of the cloned homolog. Because many plant miRNAs have heterogeneity at either the 5′ or 3′ end, the ends of the sequences listed may be considered to be approximations. Hairpin length is defined as the minimal sequence length containing the miRNA, miRNA*, and intervening sequence.

Additional members of these families were found by searching the Arabidopsis genome for near matches (0 to 3) to the miRNAs of these 81 loci (FIG. 20A, Step 9). After manual inspection for potential hairpin-like secondary structures, this identified six additional loci in miRNA families that were conserved to Oryza. These de novo miRNA-finding algorithm found 88% of these, and 93% of those with Oryza homologs. These Arabidopsis genes corresponded to 122 Oryza miRNA genes, of which 111 (91%) were found de-novo by this algorithm (FIG. 22A, Step 9; FIG. 29).

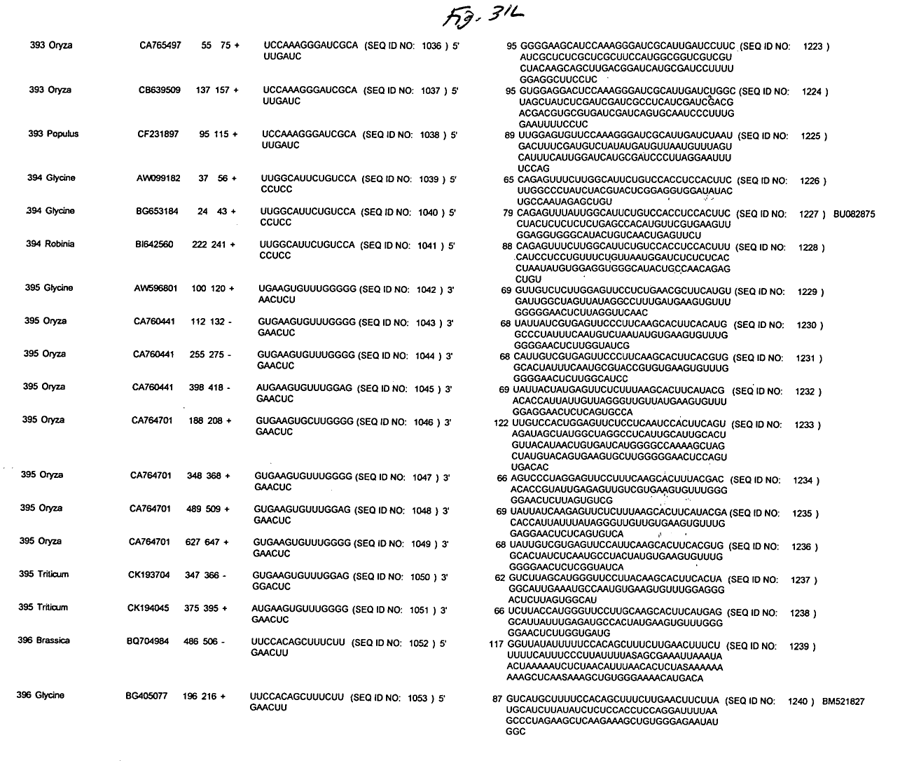

Some of the plant miRNA genes were clustered in the genome, most strikingly the genes of the 395 family. In Arabidopsis, miRNAs of the 395 family were located in two clusters, each containing three hairpins within 4 kb (FIG. 30A). In each cluster, two M!R395 hairpins were on one strand while the third is on the opposite. Thus each cluster could not be expressed as a single primary transcript, but could be expressed as two transcripts sharing common regulatory elements. The Oryza MIR395 hairpins were also clustered, but with a different arrangement than in Arabidopsis. The two largest Oryza MIR395 clusters contained seven and six hairpins, respectively, within 1 kb, with all hairpins encoded on the same strand of DNA (FIG. 30B). These clusters may be expressed as transcripts containing multiple miRNAs, an idea supported by Oryza EST CA764701, which contains four miR395 hairpins.

The above-described computational methods were applied to the prediction of conserved mRNA targets of certain known Arabidopsis and Oryza miRNAs (FIG. 25). FIG. 25 shows predicted miRNA targets with scores of 3.0 or less in both Arabidopsis and Oryza. The score of the best scoring 20-mer from any member of the miRNA family to each gene is given in parentheses. Predicted targets with scores greater than 3.0 in either Arabidopsis or Oryza but have been validated by 5′-RACE are also listed and marked with an asterisks. Underlined genes were validated as miRNA targets by 5′-RACE experiments.

Control experiments with refset2 and 5 sets of permuted miRNAs suggested that a score cutoff of ≦3.5 was appropriate to identify conserved miRNA targets with high sensitivity and 30 selectivity. However, when searching for targets of the entire set of miNRAs, this cutoff identified a number of mRNAs for which miRNA mediated cleavage products could not be found by 5′-RACE. Thus, a cutoff of ≦3 was chosen to minimize the number of non-authentic targets. The previously validated targets miRNA targets were identified at this level of sensitivity, although several newly validated targets had scores of 3.5 in one or both species and are not retained using this cutoff. A score of <3.0 in this method identified targets with very high confidence.

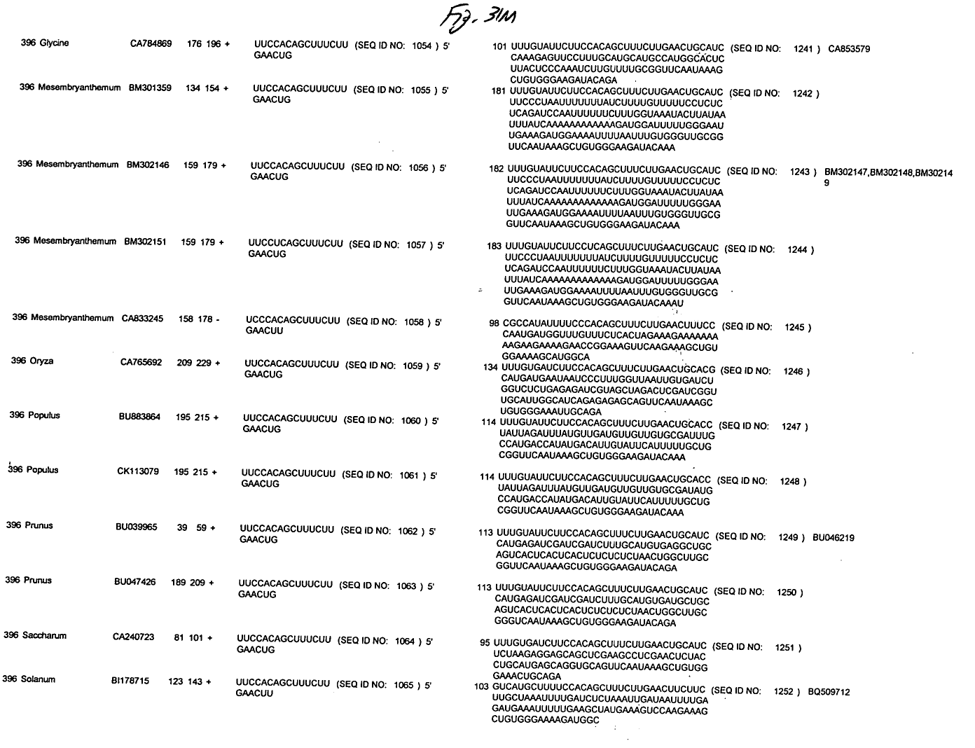

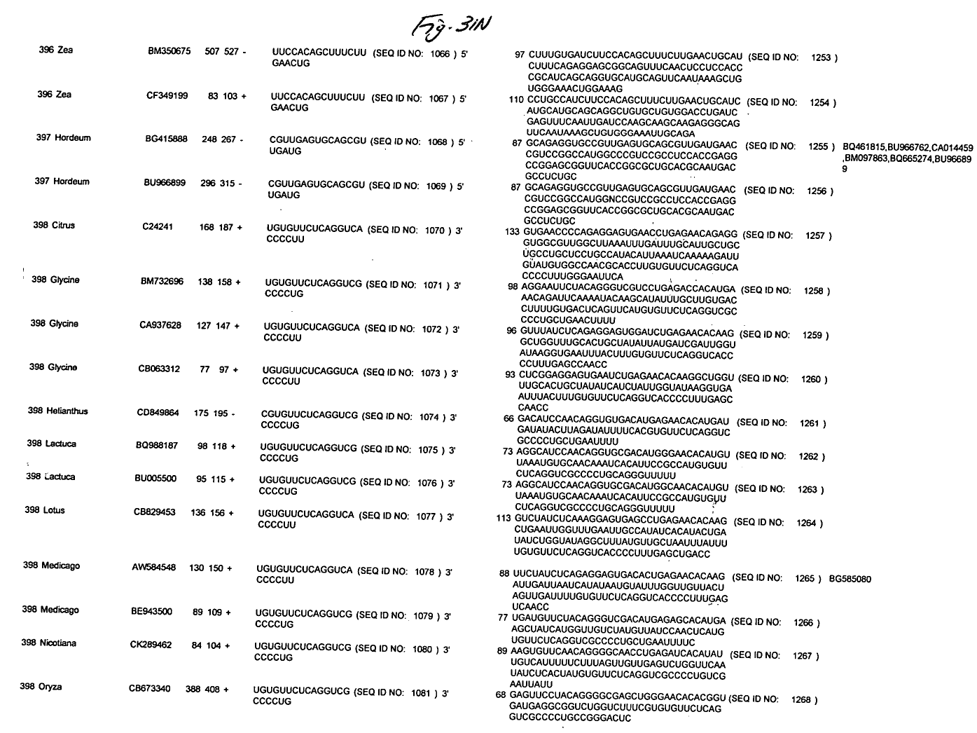

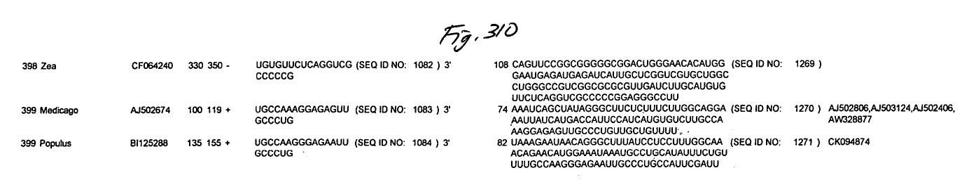

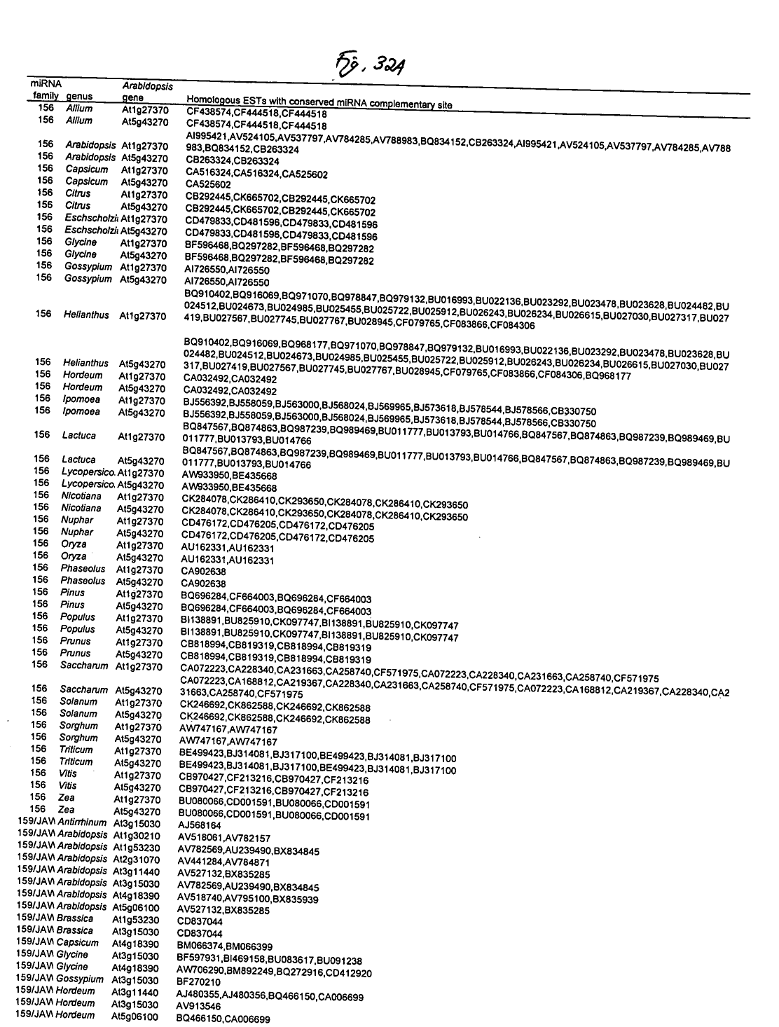

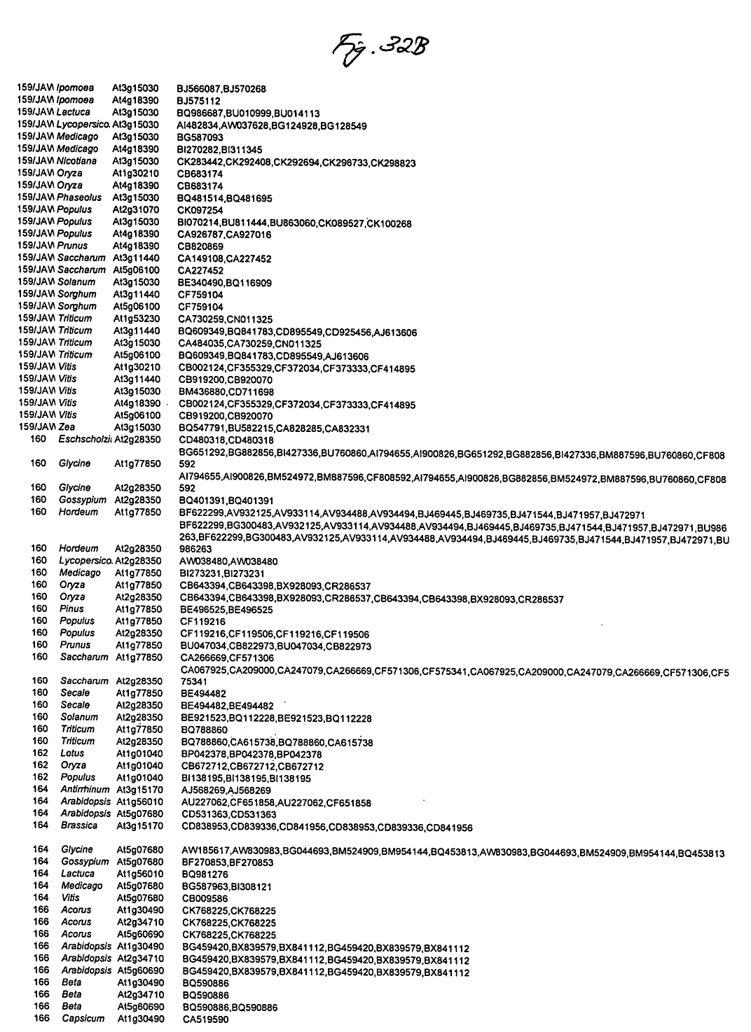

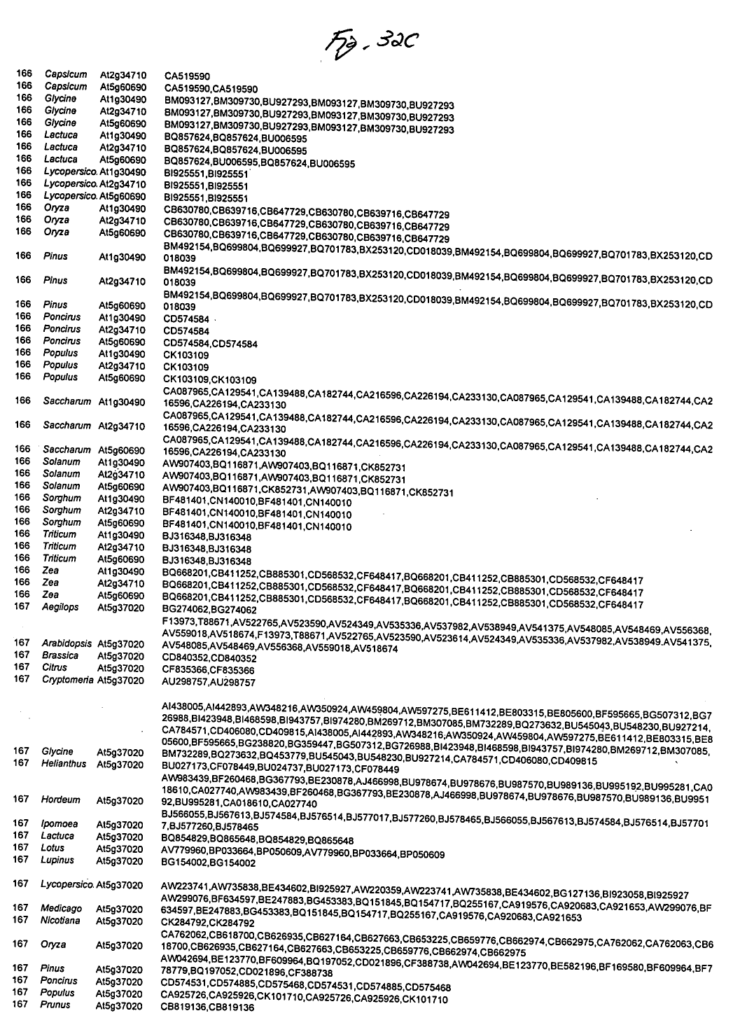

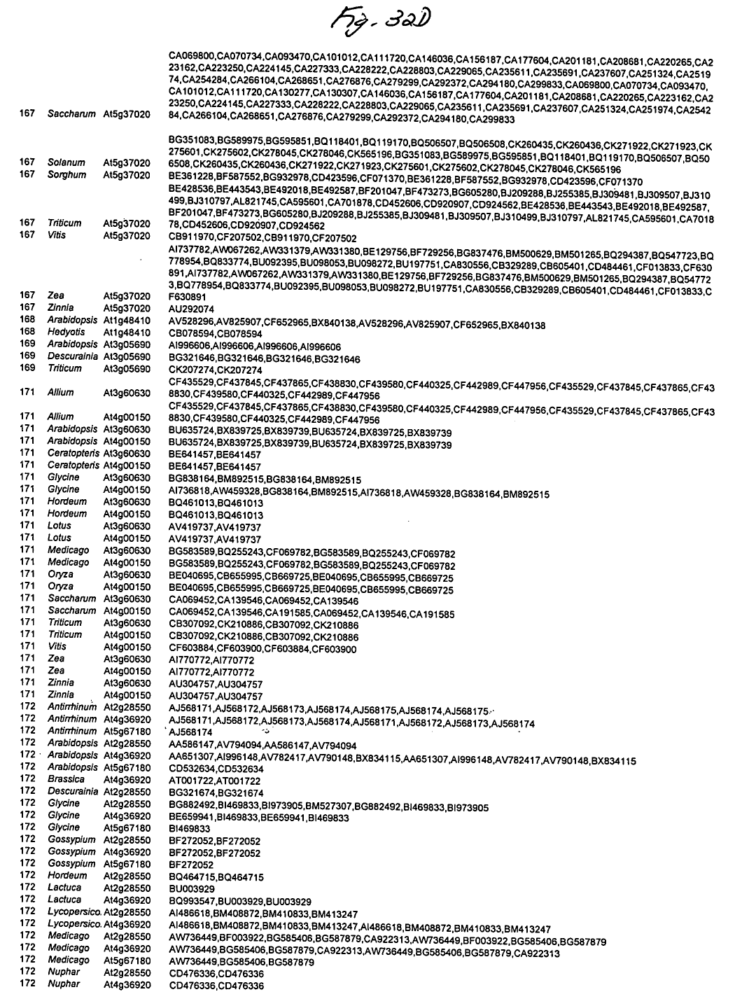

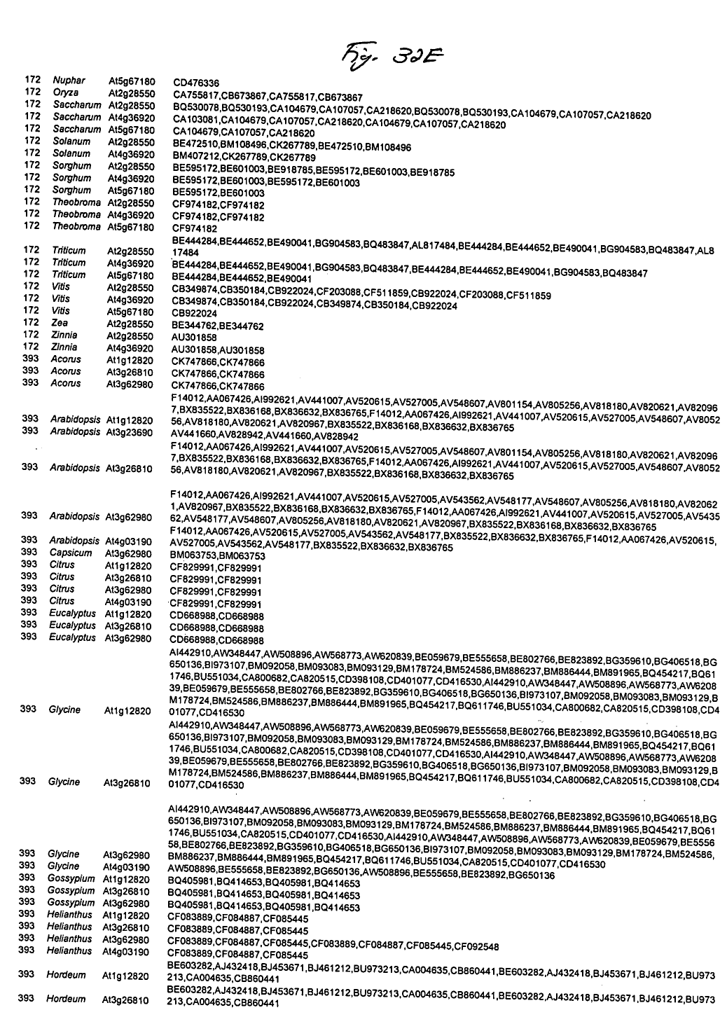

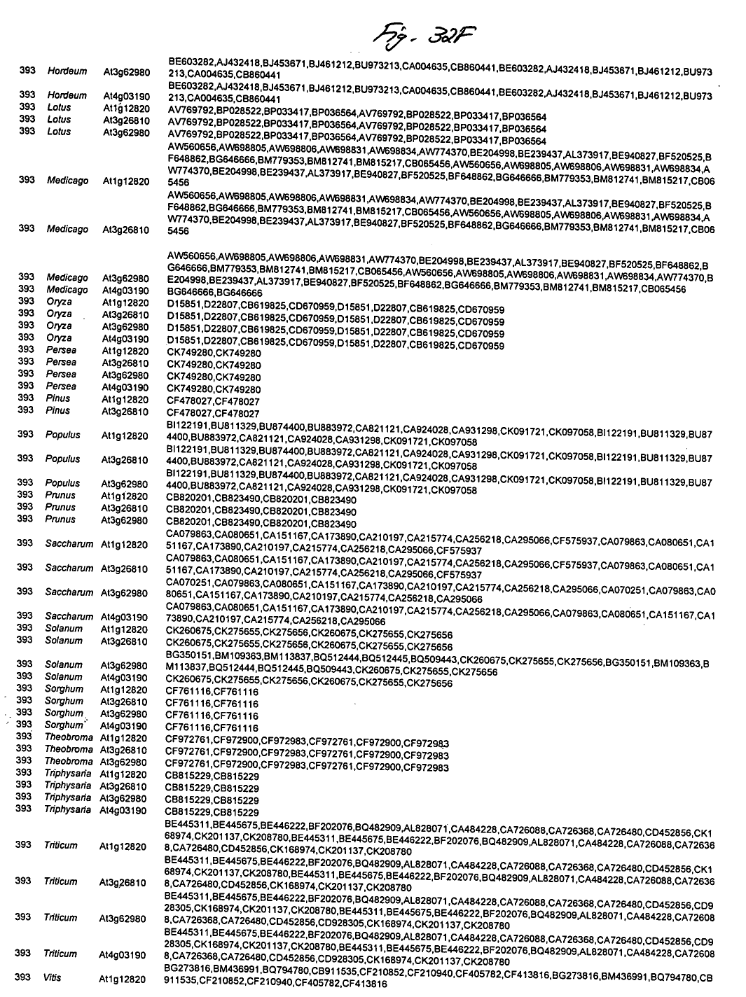

MicroRNAs were conserved between the dicot Arabidopsis thaliana and the monocot Oryza sativa, and can be found in most flowering plants. Homologs of miR-JAW and miR-JAW complementary sites have been found in ESTs from numerous angiosperms. ESTs representing potential homologs of Arabidopsis and Oryza miRNAs were also searched in this example, defined here as having 19/20 nucleotide matches and a predicted foldback that passes MIRcheck. This search identified 187 putative miRNA homologs in the ESTs (FIG. 31).

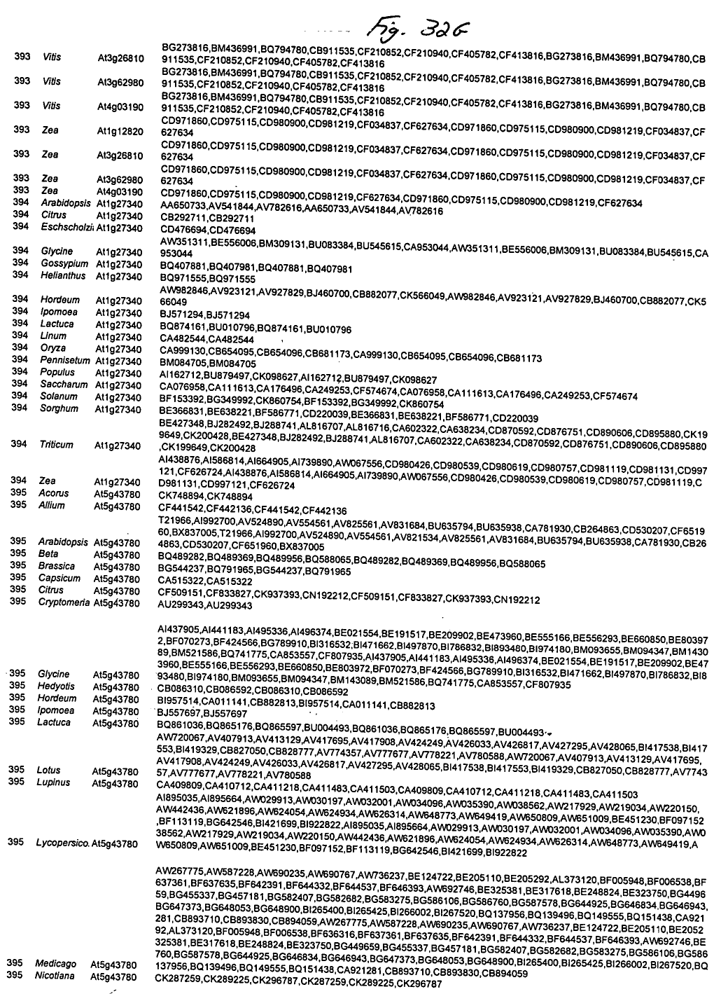

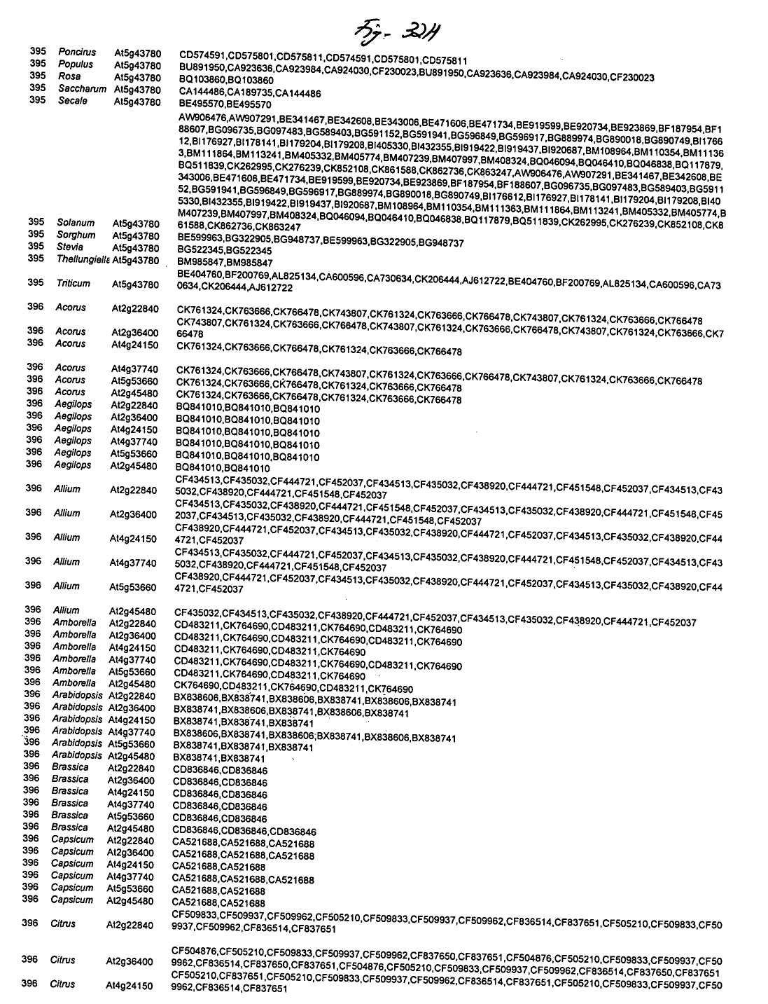

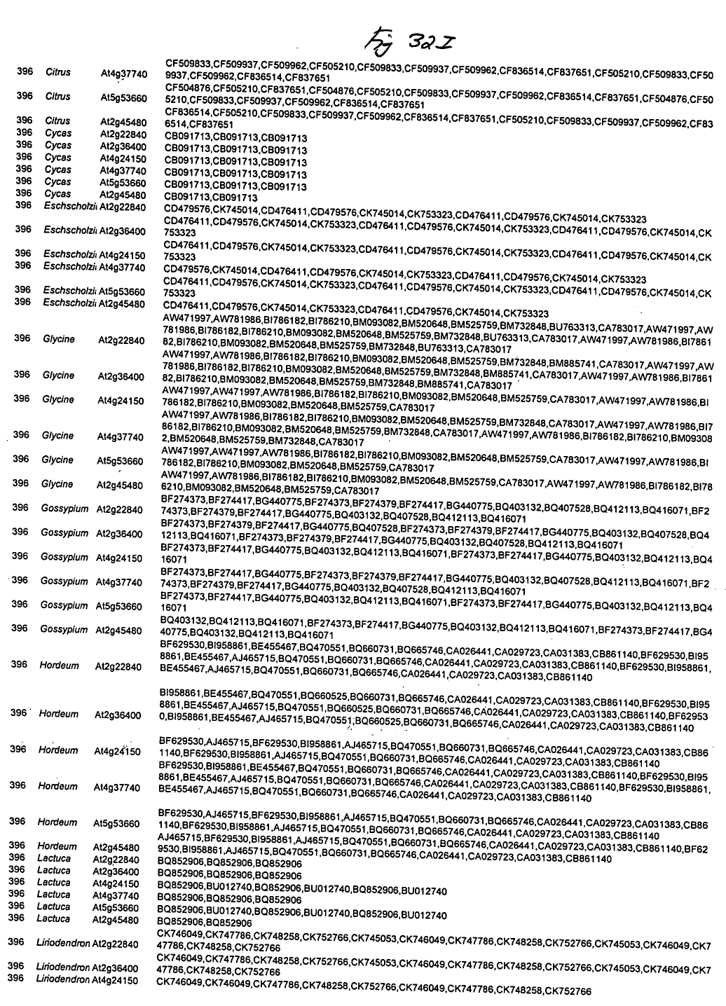

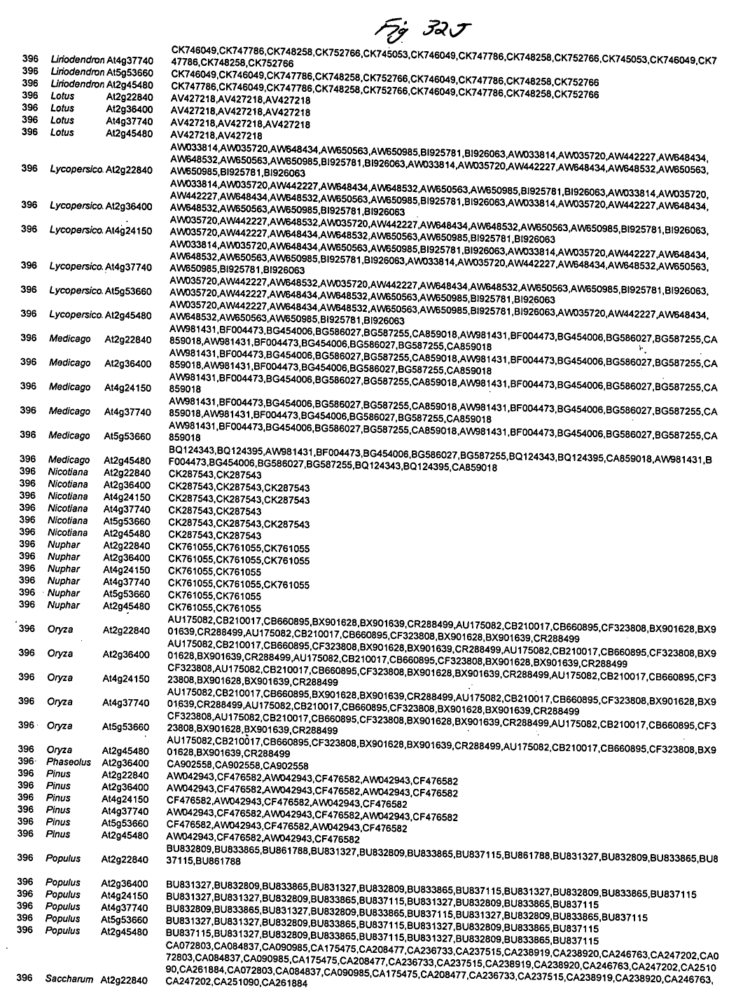

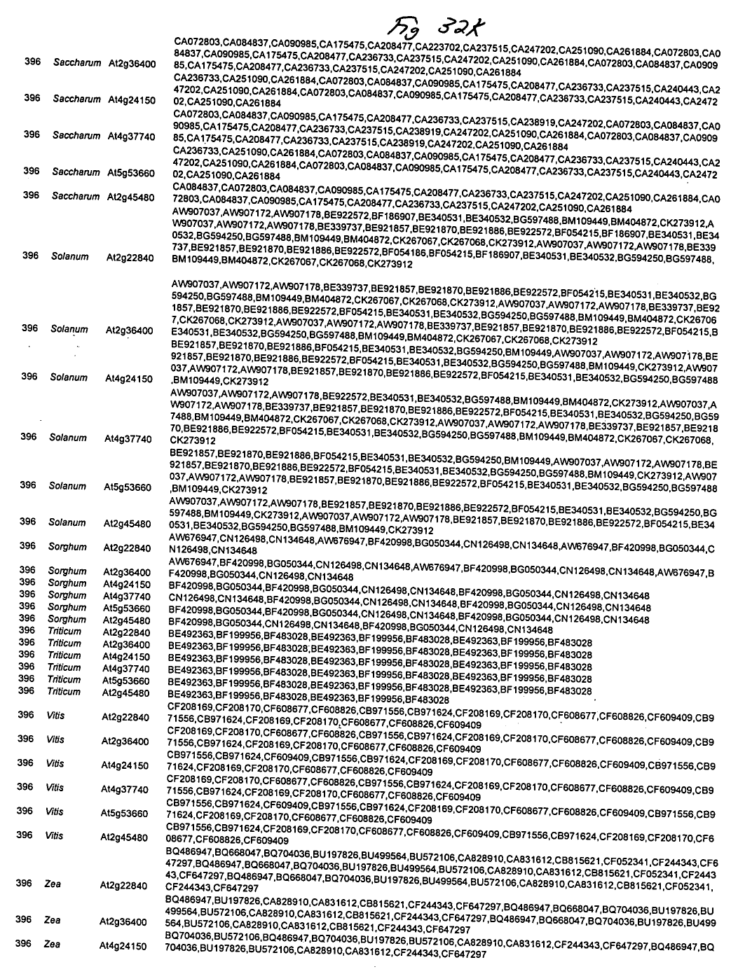

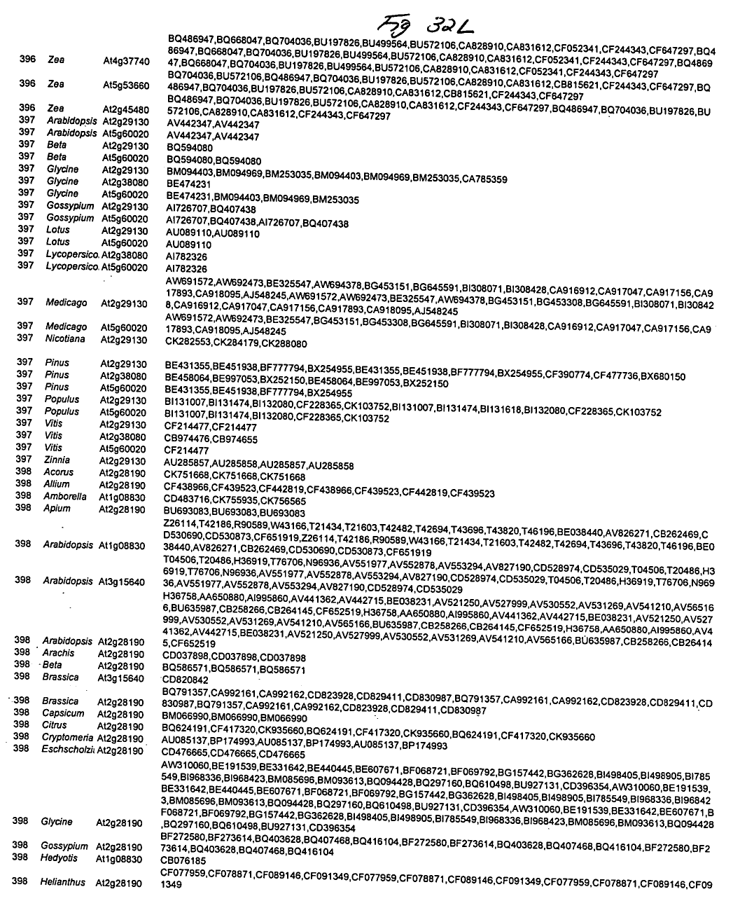

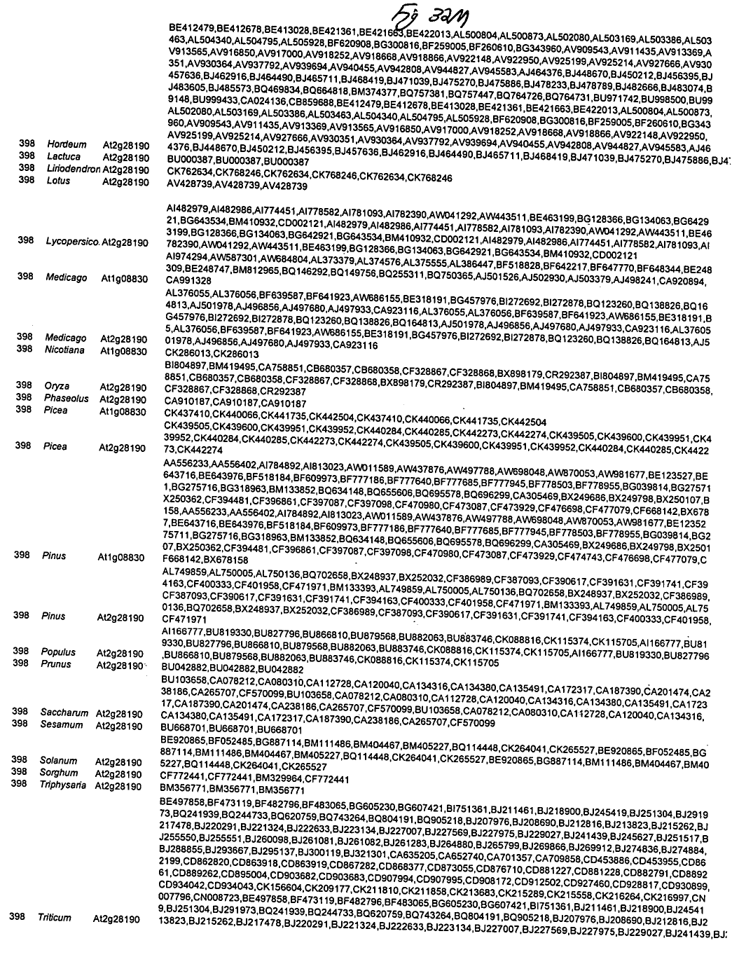

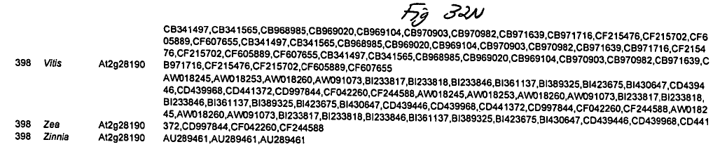

The 10 miRNAs in refset2 each had on average 9.7 EST matches that passed MIRcheck, whereas the set of 50 permuted miRNAs averaged only 0.04 matches that passed MIRcheck. For all 18 miRNA families that were conserved between Arabidopsis and Oryza, potential miRNA precursors were found in at least one additional angiosperm species (FIG. 31). For miRNAs that were not conserved between Arabidopsis and Oryza, no homologous miRNAs in additional species were identified, showing that the lack of conservation in Oryza is a consequence of recent emergence rather than loss in the Oryza lineage. Matches to experimentally confirmed miRNA complementary sites were also searched in ESTs encoding proteins homologous to Arabidopsis targets (blastx score >10-6). For all miRNA families with validated miRNA targets, conserved miRNA complementary sites (19/20 nucleotide matches) were found in at least one additional angiosperm (FIG. 32). In FIG. 32, near matches (19/20 nucleotide matches) to miRNA complementary sites of confirmed Arabidopsis miRNA targets were found in non-human, non-mouse ESTs in the Apr. 5, 2004 release of dbEST from NCBI. Complementary sites were considered to be onserved if the EST encodes a protein with homology (blastx E value <10-6) to the protein encoded by the Arabidopsis target.

On average, the miRNA complementary sites from 17 unrelated Arabidopsis miRNA targets were each conserved in 191 homologous ESTs, representing 14 species. This is far more than would be expected by chance; when repeating the analysis using 170 sites chosen at random from the same Arabidopsis mRNAs, the average number of ESTs and species were 2.6 and 0.5, respectively.

MicroRNAs of the 166 family, as well as their binding sites in mRNAs of HD-ZIP proteins, predate the emergence of seed plants. Nine miRNA families (156, 160, 166, 167, 393, 395, 396, 397 and 398) that had complementary sites conserved in gymnosperms, while a miR171 complementary site was conserved in a SCL mRNA from a fern (Ceratopteris richarii). In addition, an miRNA hairpin of the 159/JAW family was present in an EST from moss (Physomitrella patens). These data suggest that multiple miRNAs have deep origins in plant phylogeny.

Families 393, 394, 395 and 396 were absent from the reported sets of cloned, sequenced small RNAs. These were each detectable by Northern analysis, and as with families 397, 398 and 399 were detected by PCR. They may represent miRNAs that were needed at low levels, or whose expression is limited to rare cell types or particular growth conditions. The expression of miR395 may be greatly increased by sulfate starvation; other miRNAs with seemingly low expression may also be inducible by metabolite levels or environmental stimuli. It is the identification of these miRNAs that makes computational prediction a useful method of to cloning of small RNAs.

Some of the newly identified targets resemble those of previous predictions with regard to their proven or inferred roles in regulating developmental processes (FIG. 25). miR396 targets seven Growth Regulating Factor genes, which may be transcription factors that regulate cell expansion in leaf and cotyledon. miR393 and miR394 both target the messages of F-box proteins, which in turn target specific proteins for proteolysis by making them substrates for ubiquitination by SCF E3 ubiquitin ligases. At2g27340, targeted by miR394, is in the same subfamily of F-box genes as UNUSUAL FLORAL ORGANS (UFO), which is involved in floral initiation and development. miR393 targets four closely related F-box genes, including TRANSPORT INHIBITOR RESPONSE1 (TIR1), which targets AUX/IAA proteins for proteolysis in an auxin-dependent manner and is necessary for auxin-induced growth processes.

The identification of TIR1 as a miRNA target implies that miRNAs regulate auxin-responsiveness at multiple points. Other auxin related miRNA targets include Auxin Response Factors (miR160 and miR167), which are thought to regulate transcription in response to auxin, and NAC1 (miR164), which promotes auxin-induced lateral root growth downstream of TIR1. Finally, in addition to targeting F-box genes, miR393 also targets At3g23690, a basic helix-loop-helix transcription factor with homology to GBOF-1 from tulip, which Genbank. annotates as auxin-inducible.

Other newly identified miRNA targets have less obvious connections to the control of developmental patterning (FIG. 25). miR397 targets putative laccases, members of a family of enzymes with numerous described roles in fungal biology but without well defined roles in plant biology. miR399 targets two copper superoxide dismutases, CSD1 and CSD2, enzymes which protect the cell against radicals and whose expression patterns respond to oxidative stress.

The most definitive example of a plant miRNA operating outside the gene regulatory circuitry controlling development is miR395. miR395 targets the ATP sulfurylases, APS1, APS3 and APS4, enzymes that catalyze the first step of inorganic sulfate assimilation. The observations that the expression of miR395 depends on sulfate concentration and that APS1 expression declines with increasing miR395 corroborate the idea that this miRNA regulates sulfate metabolism (FIG. 24).

An overwhelming propensity for targeting messages of known or suspected plant transcription factors was found (63 of 83, or 76% of genes in FIG. 25) and similar propensity for targeting messages of genes with known or suspected roles in plant development (70 of 83, or 84% of genes in FIG. 25). The conserved targets of plant miRNAs may extend beyond the regulatory circuitry of development. The discovery that miRNAs regulate genes such as ATP sulfurylases, laccases, and superoxide dismutases shows that miRNAs also have an ancient role in regulating other aspects of plant biology.

In summary, the sensitivity of this computational approach, which found all 11 conserved miRNA families previously identified through cloning, suggests that many plant miRNAs with properties similar to previously cloned miRNAs were identified. The detection of the RNA fragments diagnostic of miRNA-directed cleavage confirms in planta these identified miRNA-target interactions. For many of the other plant miRNA targets examined, inhibition of the miRNA pathway leads to increased accumulation of target mRNA, showing that mRNA cleavage typically plays a significant regulatory role.

The entire contents of all of references (including literature references, issued patents, published patent applications, and co-pending patent applications) cited throughout the specification are hereby incorporated by reference.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving, “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition, comprising:
an isolated nucleotide sequence able to be transcribed by a plant cell into precursor miRNA that is cleavable by the plant cell to produce miRNA substantially complementary to at least a portion of an mRNA sequence encoding a gene.

2-7. (canceled)

8. The composition of claim 1, wherein the precursor miRNA has a structure: 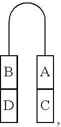
wherein 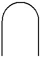
is a nucleotide sequence comprising a stem-loop motif, 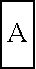
is a nucleotide sequence expressable as miRNA in the plant cell, 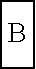
is a nucleotide sequence substantially complementary to 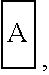
and each of 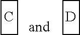
independently is either absent or is a nucleotide sequence having at least one nucleotide.

9. (canceled)

10. The composition of claim 8, wherein 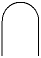
comprises a nucleotide sequence selected from the group consisting of SEQ ID NO: 17 to SEQ ID NO: 53 and SEQ ID NO: 62 to SEQ ID NO: 89, inclusive, or a variant thereof comprising 1-40 nucleotide mismatches.

11-14. (canceled)

15. The composition of claim 8, wherein 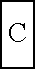
is substantially complementary to

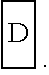

16-21. (canceled)

22. The composition of claim 1, wherein the miRNA comprises a nucleotide sequence selected from the group consisting of SEQ ID NO:1 to SEQ ID NO: 16, inclusive, or a variant thereof comprising 1-7 nucleotide mismatches.

23-25. (canceled)

26. A method of inhibiting a gene, comprising:
replacing at least a portion of a nucleotide sequence, able to be transcribed by a plant cell into precursor miRNA cleavable by the plant cell to produce miRNA, with a sequence substantially complementary to a gene to be inhibited; and
contacting the plant cell with the nucleotide to inhibit gene expression.

27. The method of claim 26, wherein the precursor miRNA has a structure: 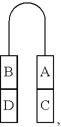
wherein 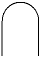
is a nucleotide sequence comprising a stem-loop motif, 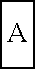
is a nucleotide sequence expressable as miRNA in the plant cell, 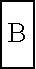
is a nucleotide sequence substantially complementary to 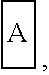
and each of 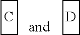
independently is either absent or is a nucleotide sequence having at least one nucleotide.

28. The method of claim 27, wherein 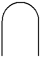
comprises a nucleotide sequence selected from the group consisting of SEQ ID NO:17 to SEQ ID NO: 53, inclusive, or a variant thereof comprising 1-40 nucleotide mismatches.

29. The method of claim 27, wherein 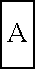
comprises a nucleotide sequence selected from the group consisting of SEQ ID NO:1 to SEQ ID NO: 16, inclusive, or a variant thereof comprising 1-7 nucleotide mismatches.

30-32. (canceled)

33. A method of inhibiting a gene, comprising:
replacing a portion of a precursor miRNA taken from a plant cell with a sequence substantially complementary to a gene to be inhibited; and
contacting the plant cell with the precursor miRNA to inhibit gene expression.

34. The method of claim 33, wherein the precursor miRNA has a structure: 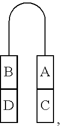
wherein 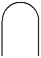
is a nucleotide sequence comprising a stem-loop motif, 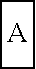
is a nucleotide sequence expressable as miRNA in the plant cell, 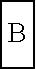
is a nucleotide sequence substantially complementary to 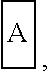
and each of 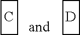
independently is either absent or is a nucleotide sequence having at least one nucleotide.

35. The method of claim 34, wherein 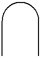
comprises a nucleotide sequence selected from the group consisting of SEQ ID NO:17 to SEQ ID NO: 53, inclusive, or a variant thereof comprising 1-40 nucleotide mismatches.

36. The method of claim 34, wherein 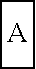
comprises a nucleotide sequence selected from the group consisting of SEQ ID NO:1 to SEQ ID NO: 16, inclusive, or a variant thereof comprising 1-7 nucleotide mismatches.

37-39. (canceled)

40. A composition, comprising:
an isolated precursor miRNA able to inhibit a gene in a plant cell.

41. (canceled)

42. The composition of claim 40, wherein the precursor miRNA has a structure: 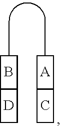
wherein 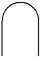
is a nucleotide sequence comprising a stem-loop motif, 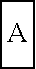
is a nucleotide sequence expressable as miRNA in the plant cell, 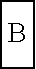
is a nucleotide sequence substantially complementary to 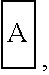
and each of 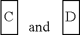
independently is either absent or is a nucleotide sequence having at least one nucleotide.

43. The composition of claim 42, wherein 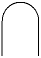
comprises a nucleotide sequence selected from the group consisting of SEQ ID NO:17 to SEQ ID NO: 53, inclusive, or a variant thereof comprising 1-40 nucleotide mismatches.

44. The composition of claim 42, wherein 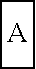
comprises a nucleotide sequence selected from the group consisting of SEQ ID NO:1 to SEQ ID NO: 16, inclusive, or a variant thereof comprising 1-7 nucleotide mismatches.

45-47. (canceled)

48. A composition, comprising:
isolated plant-derived miRNA.

49-50. (canceled)

51. The composition of claim 48, wherein the isolated plant-derived miRNA comprises a nucleotide sequence selected from the group consisting of SEQ ID NO:1 to SEQ ID NO: 16, inclusive, or a variant thereof comprising 1-7 nucleotide mismatches.

52. A method, comprising:
altering expression of miRNA in a plant cell by altering an environmental condition surrounding the plant cell.

53-54. (canceled)


SEQUENCE LISTING (1319 sequences)

SEQ ID NO: 1 - RNA, 20 nt, Arabidopsis thaliana
ugacagaagagagugagcac

SEQ ID NO: 2 - RNA, 21 nt, Arabidopsis thaliana
uugacagaagauagagagcac

SEQ ID NO: 3 - RNA, 20 nt, Arabidopsis thaliana
ucccaaauguagacaaagca

SEQ ID NO: 4 - RNA, 21 nt, Arabidopsis thaliana
uuuggauugaagggagcucua

SEQ ID NO: 5 - RNA, 21 nt, Arabidopsis thaliana
ugccuggcucccuguaugcca

SEQ ID NO: 6 - RNA, 21 nt, Arabidopsis thaliana
uugaaagugacuacaucgggg

SEQ ID NO: 7 - RNA, 21 nt, Arabidopsis thaliana
ucgauaaaccucugcauccag

SEQ ID NO: 8 - RNA, 24 nt, Arabidopsis thaliana
uugaagaggacuuggaacuucgau

SEQ ID NO: 9 - RNA, 21 nt, Arabidopsis thaliana
uggagaagcagggcacgugca

SEQ ID NO: 10 - RNA, 21 nt, Arabidopsis thaliana
ucggaccaggcuucauccccc

SEQ ID NO: 11 - RNA, 21 nt, Arabidopsis thaliana
ucggaccaggcuucauucccc

SEQ ID NO: 12 - RNA, 21 nt, Arabidopsis thaliana
ugaagcugccagcaugaucua

SEQ ID NO: 13 - RNA, 21 nt, Arabidopsis thaliana
ucgcuuggugcaggucgggaa

SEQ ID NO: 14 - RNA, 21 nt, Arabidopsis thaliana
cagccaaggaugacuugccga

SEQ ID NO: 15 - RNA, 21 nt, Arabidopsis thaliana
ugauugagccgugucaauauc

SEQ ID NO: 16 - RNA, 21 nt, Arabidopsis thaliana
ugauugagccgcgccaauauc

SEQ ID NO: 17 - RNA, 40 nt, Arabidopsis thaliana
acaaaggcaauuugcauaucauugcacuugcuucucuugc

SEQ ID NO: 18 - RNA, 38 nt, Arabidopsis thaliana
augcaggcacuguuaugugucuauaacuuugcgugugc

SEQ ID NO: 19 - RNA, 41 nt, Arabidopsis thaliana
acaaaggcacuuugcauguucgaugcauuugcuucucuugc

SEQ ID NO: 20 - RNA, 45 nt, Arabidopsis thaliana
acaaaggggaaguuguauaaaaguuuuguauaugguugcuuuugc

SEQ ID NO: 21 - RNA, 55 nt, Arabidopsis thaliana
acauggugguuucuugcaugcuuuuuugauuaggguuucaugcuugaagcuaugu

SEQ ID NO: 22 - RNA, 49 nt, Arabidopsis thaliana
acaugguggcuuucuugcauauuugaagguuccaugcuugaagcuaugu

SEQ ID NO: 23 - RNA, 48 nt, Arabidopsis thaliana
agaugaugagauacaauucggagcauguucuuugcaucuuacuccuuu

SEQ ID NO: 24 - RNA, 48 nt, Arabidopsis thaliana
agaugauaagauacaauuccucgcagcuucuuugcaucuuacuccuuu

SEQ ID NO: 25 - RNA, 123 nt, Arabidopsis thaliana
uaaggaugacaugcaaguacauacauauauaucaucacaccgcauguggaugauaaaauauguauaacaaauucaaagaaagagagggagagaaagagagagaaccugcaucucuacucuuuu

SEQ ID NO: 26 - RNA, 129 nt, Arabidopsis thaliana
uaaggaugcuauugcaaaacagacacagauauguguuucuaauuguauucauacuuuaaccucaaaguugauauaaaaaaagaaagaaagauagaagagcuagaagacuaucugcaucucuauuccuau

SEQ ID NO: 27 - RNA, 24 nt, Arabidopsis thaliana
aaaagugaugacgccauugcucuu

SEQ ID NO: 28 - RNA, 140 nt, Arabidopsis thaliana
caugaguugagcaggguaaagaaaagcugcuaagcuauggaucccauaagcccuaauccuuguaaaguaaaaaaggauuugguuauauggauugcauaucucaggagcuuuaacuugcccuuuaauggcuuuuacucuuc

SEQ ID NO: 29 - RNA, 37 nt, Arabidopsis thaliana
uaugcugagcccaucgaguaucgaugaccuccgugga

SEQ ID NO: 30 - RNA, 38 nt, Arabidopsis thaliana
caagaaaacaucgauuuaguuucaaaaucgaucacuag

SEQ ID NO: 31 - RNA, 39 nt, Arabidopsis thaliana
cgaguggauaccgauuuugguuuuaaaaucggcugccgg

SEQ ID NO: 32 - RNA, 48 nt, Arabidopsis thaliana
uuccgauuuuuuuuguucuucauaugaugaagcggaaacaguaaucaa

SEQ ID NO: 33 - RNA, 43 nt, Arabidopsis thaliana
ucucuuccugugaacacauuaaaaauguaaaagcaugaauaga

SEQ ID NO: 34 - RNA, 46 nt, Arabidopsis thaliana
ucaauuccugugaauauuuauuuuuguuuacaaaagcaagaaucga

SEQ ID NO: 35 - RNA, 257 nt, Arabidopsis thaliana
cgacaacgauuucaacacucucuuccaggaacaacuuccuccaggcagaugauacuaaagugcuggaguucccgguuccugagagugaguccauaucaaaaugcgcauucguuaucacuugguugaacccauuuggggauuuaaauuuggaggugaaauggaacgcguaauugaugacuccuacguggaaccucuucuuaggaagagcacggucgaagaguaacugcgcagugcuuaaaucguagaugcuaaagucg

SEQ ID NO: 36 - RNA, 36 nt, Arabidopsis thaliana
aaccaacaaacacgaaauccgucucauuugcuuauu

SEQ ID NO: 37 - RNA, 107 nt, Arabidopsis thaliana
uuacuagcucauauauacacucucaccauaaaugcguguauauaugcggaauuuugugauauagauguguguguguguugagugugaugauauggaugaguuaguuc

SEQ ID NO: 38 - RNA, 59 nt, Arabidopsis thaliana
ggauauuauagauauauacauguguauguuaaugauucaagugaucauagagaguaucc

SEQ ID NO: 39 - RNA, 94 nt, Arabidopsis thaliana
ggauaucauaaacgcauacacauguuuauauguuaugaugcauuauaugacugauguaauguacauauauauacauacaugccacaugguaucg

SEQ ID NO: 40 - RNA, 94 nt, Arabidopsis thaliana
ggacucuggcucgcucuauucauguuggaucucuuucgaucuaacaaucgaauugaaccuucagauuucagauuugauuaggguuuuagcgucu

SEQ ID NO: 41 - RNA, 70 nt, Arabidopsis thaliana
ggacucuuauucuaauacaaucucauuugaauacauucagaucugaugauugauuaggguuuuagugucg

SEQ ID NO: 42 - RNA, 66 nt, Arabidopsis thaliana
ggucaugaagaagagaaucacucgaauuaauuuggaagaacaaauuaagaaaacccuagaugauuc

SEQ ID NO: 43 - RNA, 59 nt, Arabidopsis thaliana
ggucaugaagaagaucgguagauugauucauuuuaaagagugaaaucccuaaaugauuc

SEQ ID NO: 44 - RNA, 93 nt, Arabidopsis thaliana
ggcccuuaacuuagaucuauauuugauuauauauauaugucucuucuuuauucauuagucuauacaugaaugaucauuuuacgguuaaugacg

SEQ ID NO: 45 - RNA, 49 nt, Arabidopsis thaliana
gaccauucaaucucaugaucucaugauuauaacgaugaugaugaugaug

SEQ ID NO: 46 - RNA, 48 nt, Arabidopsis thaliana
ggucauggagaguaauucguuaacccaacucaaaacucuaaaugauuc

SEQ ID NO: 47 - RNA, 59 nt, Arabidopsis thaliana
auuagcuuucuuuauccuuuguuguguuucaugacgaugguuaagagaucagucucgau

SEQ ID NO: 48 - RNA, 47 nt, Arabidopsis thaliana
ucuuugguuaagagaugaauguggaaacauauugcuuaaacccaagc

SEQ ID NO: 49 - RNA, 62 nt, Arabidopsis thaliana
ccaauucggcugacacagccgacuuuuaaaccuuuauugguuugugagcauggucggauugg

SEQ ID NO: 50 - RNA, 48 nt, Arabidopsis thaliana
cugauuggcugacaccgacacgugucuugucaugguugguuugugagc

SEQ ID NO: 51 - RNA, 148 nt, Arabidopsis thaliana
uuuaaaugaucuuucuuuauacucuauuaagacaauuuaguuucaaacuuuuuuuuuuuuuuuuuuuugaaggauucaggaagaaauuaggauauauuauuccguauaaaauacaagauauauaaaaccaaaagaaaaaguaacauga

SEQ ID NO: 52 - RNA, 22 nt, Arabidopsis thaliana
gauucucuuuuaucaacucauc

SEQ ID NO: 53 - RNA, 50 nt, Arabidopsis thaliana
gaucuuaccugaccacacacguagauauacauuauucucucuagauuauc

SEQ ID NO: 54 - RNA, 20 nt, Oryza sativa
ugacagaagagagugagcac

SEQ ID NO: 55 - RNA, 21 nt, Oryza sativa
ugccuggcucccuguaugcca

SEQ ID NO: 56 - RNA, 21 nt, Oryza sativa
ucgauaaaccucugcauccag

SEQ ID NO: 57 - RNA, 21 nt, Oryza sativa
uggagaagcagggcacgugca

SEQ ID NO: 58 - RNA, 21 nt, Oryza sativa
ucggaccaggcuucauucccc

SEQ ID NO: 59 - RNA, 21 nt, Oryza sativa
ugaagcugccagcaugaucua

SEQ ID NO: 60 - RNA, 21 nt, Oryza sativa
cagccaaggaugacuugccga

SEQ ID NO: 61 - RNA, 21 nt, Oryza sativa
ugauugagccgcgccaauauc

SEQ ID NO: 62 - RNA, 47 nt, Oryza sativa
acgugguuguuuccuugcauaaaugaugccuaugcuuggagcuacgc

SEQ ID NO: 63 - RNA, 45 nt, Oryza sativa
acacggugcuuucuuagcaugcaagagccaugcugggagcugugc

SEQ ID NO: 64 - RNA, 49 nt, Oryza sativa
acauggugacuuucuugcaugcugaauggacucaugcuugaagcuaugu

SEQ ID NO: 65 - RNA, 42 nt, Oryza sativa
acagcgugauggccggcauaaaaucuaucccguccucgccgc

SEQ ID NO: 66 - RNA, 44 nt, Oryza sativa
acggccgggcgugacggcaccggcgggcgugccgucgcggccgc

SEQ ID NO: 67 - RNA, 140 nt, Oryza sativa
acagcggccagacugcaucgaucuaucaaucuucccuucgacaggauaacuagguagaaagaaagagaggccgucggcggccauggaagagagagagagagagagagaugaaugaugaugaugauacagcugccgcucgc

SEQ ID NO: 68 - RNA, 70 nt, Oryza sativa
acagcgggcagacugcaucugaaauaaacuggugacgacgaagaagacgacggacgcagcuugccguugc

SEQ ID NO: 69 - RNA, 44 nt, Oryza sativa
acggcgcggcggcuagccaucggcgggaugccugcccccgccgc

SEQ ID NO: 70 - RNA, 44 nt, Oryza sativa
cgccggcgcugccguguaggcggccacggcaaggcgggccggca

SEQ ID NO: 71 - RNA, 44 nt, Oryza sativa
cgccgcccccguccguagggcggcuaccgaucggcggcgcggca

SEQ ID NO: 72 - RNA, 40 nt, Oryza sativa
cacauguagaccaacccauggugucugguugccuacuggg

SEQ ID NO: 73 - RNA, 40 nt, Oryza sativa
cucauguagcccaauccaugguguguuuggaugcuguggg

SEQ ID NO: 74 - RNA, 45 nt, Oryza sativa
cucaucuagagcaacaaacuucugcgagagguugccuaugaugga

SEQ ID NO: 75 - RNA, 88 nt, Oryza sativa
cucgcguagcugccaaacucaguugaaacaacugccuucucccggcgagauucaggcauuguguucguacguuuggcucuacugcgga

SEQ ID NO: 76 - RNA, 114 nt, Oryza sativa
ucccuucccugccuuguggcgcugauccaggagcggcgaauuucuuugagaggguguucuuuuuuuuuucuuccuuuugguccuuguugcagccaacgacaacgcgggaaucga

SEQ ID NO: 77 - RNA, 79 nt, Oryza sativa
ugcauauguucaucaucaucuucuuccuccuccucuagcuccagccuuguguggguuggaaguuuagauagaacucgca

SEQ ID NO: 78 - RNA, 51 nt, Oryza sativa
uuaccauccacucgccugccggccgccggccgccauugccauggaugguuc

SEQ ID NO: 79 - RNA, 69 nt, Oryza sativa
ggucucauacaccuugugguuuugaggaugauuugugcaagguuuuucauuccucucauccgugggauc

SEQ ID NO: 80 - RNA, 128 nt, Oryza sativa
ggcuacuuuuaauuucucucucuuuugauaucuucuuuucucgaucuccuagcuugaucuuuuugaucucucaaaucgaucuuaagaaaaagaucagucaaagagaugagaguagaugucuguagauc

SEQ ID NO: 81 - RNA, 60 nt, Oryza sativa
gaccuaacaccgggcggaauggcggauucagcugcagcuaagcaagcuaggugggggguu

SEQ ID NO: 82 - RNA, 51 nt, Oryza sativa
ggugcauggagaaaccucugaucgaucagguuugaucuguagagacugauc

SEQ ID NO: 83 - RNA, 77 nt, Oryza sativa
ggccccuuaggaugugugauuuuugaugguuuaugcauucaucuugaugcgaacaucuaucucggaucuuuggguuc

SEQ ID NO: 84 - RNA, 35 nt, Oryza sativa
gaucguaccauagugguggguacacguggacgguc

SEQ ID NO: 85 - RNA, 72 nt, Oryza sativa
gcucugauuaaucggcacuguuggcguacagucgauugacuaaucgucagaucuguguguguaaaucacugu

SEQ ID NO: 86 - RNA, 83 nt, Oryza sativa
acuugcagacaagaaaucagcucagcucgcugguuucgaacaggaaggcggcuagcugaggcuucuucugaguacgugauggu

SEQ ID NO: 87 - RNA, 83 nt, Oryza sativa
gcucugaaugaucaacaagaugugcucccacacugccuuccuguggaucuugagcuguugcuagucuuguggucaugccuugc

SEQ ID NO: 88 - RNA, 53 nt, Oryza sativa
ucgaucgaucuaucuaugaagcuaagcuagcuggccauggauccauccaucaa

SEQ ID NO: 89 - RNA, 36 nt, Oryza sativa
gaugauugguuuuacagcagugguaaaaucaguauc

SEQ ID NO: 90 - RNA, 21 nt, Arabidopsis thaliana
uucaauaaauaauugguucua

SEQ ID NO: 91 - RNA, 21 nt, Arabidopsis thaliana
gaacuagaaaagacauuggac

SEQ ID NO: 92 - RNA, 22 nt, Arabidopsis thaliana
uccaaugucuuuucuaguucgu

SEQ ID NO: 93 - RNA, 22 nt, Arabidopsis thaliana
agaguaagauggaucuugauaa

SEQ ID NO: 94 - RNA, 22 nt, Arabidopsis thaliana
uauaucccauuucuaccaucug

SEQ ID NO: 95 - RNA, 21 nt, Arabidopsis thaliana
uccaagcgaaugaugauacuu

SEQ ID NO: 96 - protein, 46 aa, Arabidopsis thaliana
TLAEFLSKATGTAVDWVQMPGMKPGPDSVGIFAISQRCNGVAARAC

SEQ ID NO: 97 - protein, 46 aa, Arabidopsis thaliana
TLTEFISKATGTAVEWVQMPGMKPGPDSIGIVAISHGCTGIAARAC

SEQ ID NO: 98 - protein, 46 aa, Arabidopsis thaliana
TLAEFLCKATGTAVDWVQMIGMKPGPDSIGIVAVSRNCSGIAARAC

SEQ ID NO: 99 - protein, 46 aa, Arabidopsis thaliana
ALAEFLSKATGTAVDWVQMIGMKPGPDSIGIVAISRNCSGIAARAC

SEQ ID NO: 100 - protein, 46 aa, Arabidopsis thaliana
SSRTASLCERMTSCIHDSDCALSLLSSSSSSVPHLLQPPLSLSQEA

SEQ ID NO: 101 - protein, 46 aa, Arabidopsis thaliana; identical to SEQ ID NO: 100

SEQ ID NO: 102 - protein, 46 aa, Arabidopsis thaliana
PDKGVGECSGGLHESHDFYSALSLLSTTSDSQGIKHTPVAEPPPIF

SEQ ID NO: 103 - protein, 46 aa, Arabidopsis thaliana
HGEDVGEYSGVLHESQDIHRALSLLSTSSDPLAQPHVQPFSLLCSY

SEQ ID NO: 104 - protein, 46 aa, Arabidopsis thaliana
FSKEKVTISSHMGASQDLDGALSLLSNSTTWVSSSDQPRRFTLDHH

SEQ ID NO: 105 - protein, 46 aa, Arabidopsis thaliana
SSSFTTCPEMINNNSTDSSCALSLLSNSYPIHQQQLQTPTNTWRPS

SEQ ID NO: 106 - protein, 46 aa, Arabidopsis thaliana
SPEIMDTKLESYKGIGDSNCALSLLSNPHQPHDNNNNNNNNNNNNN

SEQ ID NO: 107 - protein, 46 aa, Arabidopsis thaliana
STEVSSIWDLHETAASRSTRALSLLSAQSQQHLSKFPNTTFSITQP

SEQ ID NO: 108 - RNA, 21 nt, Arabidopsis thaliana
ugugcucacucucuucuguca

SEQ ID NO: 109 - RNA, 21 nt, Arabidopsis thaliana
ugugcucucucucuucuguca

SEQ ID NO: 110 - RNA, 21 nt, Arabidopsis thaliana
ugugcucucucucuucuguca

SEQ ID NO: 111 - RNA, 21 nt, Arabidopsis thaliana
ugugcucucucucuucuguca

SEQ ID NO: 112 - RNA, 21 nt, Arabidopsis thaliana
ugugcucucucucuucuguca

SEQ ID NO: 113 - RNA, 21 nt, Arabidopsis thaliana
agugcucucucucuucuguca

SEQ ID NO: 114 - RNA, 21 nt, Arabidopsis thaliana
cgugcucucucucuucuguca

SEQ ID NO: 115 - RNA, 21 nt, Arabidopsis thaliana
ggugcucucucucuucuguca

SEQ ID NO: 116 - RNA, 21 nt, Arabidopsis thaliana
cgugcucucucucuucuguca

SEQ ID NO: 117 - RNA, 21 nt, Arabidopsis thaliana
uuugcuuacucucuucuguca

SEQ ID NO: 118 - RNA, 21 nt, Arabidopsis thaliana
ucugcucucucucuucuguca

SEQ ID NO: 119 - RNA, 21 nt, Arabidopsis thaliana
ugugcucucucucuucuguca

SEQ ID NO: 120 - RNA, 21 nt, Arabidopsis thaliana
ugugcucucucucuucuguca

SEQ ID NO: 121 - RNA, 21 nt, Arabidopsis thaliana
ugugcucucucucuucuguca

SEQ ID NO: 122 - RNA, 21 nt, Arabidopsis thaliana
ggugcucucucucuucuguca

SEQ ID NO: 123 - protein, 7 aa, Arabidopsis thaliana
CALSLLS

SEQ ID NO: 124 - protein, 7 aa, Arabidopsis thaliana
CALSLLS

SEQ ID NO: 125 - protein, 7 aa, Arabidopsis thaliana
CALSLLS

SEQ ID NO: 126 - protein, 7 aa, Arabidopsis thaliana
CALSLLS

SEQ ID NO: 127 - protein, 7 aa, Arabidopsis thaliana
SALSLLS

SEQ ID NO: 128 - protein, 7 aa, Arabidopsis thaliana
RALSLLS

SEQ ID NO: 129 - protein, 7 aa, Arabidopsis thaliana
GALSLLS

SEQ ID NO: 130 - protein, 7 aa, Arabidopsis thaliana
RALSLLS

SEQ ID NO: 131 - protein, 7 aa, Arabidopsis thaliana
CALSLLS

SEQ ID NO: 132 - protein, 7 aa, Arabidopsis thaliana
CALSLLS

SEQ ID NO: 133 - protein, 7 aa, Arabidopsis thaliana
CALSLLS

SEQ ID NO: 134 - protein, 7 aa, Arabidopsis thaliana
GALSLLS

SEQ ID NO: 135 - RNA, 21 nt, Arabidopsis thaliana
uggcauacagggagccaggca

SEQ ID NO: 136 - RNA, 21 nt, Arabidopsis thaliana
uggcaugcagggagccaggca

SEQ ID NO: 137 - RNA, 21 nt, Arabidopsis thaliana
aggaauacagggagccaggca

SEQ ID NO: 138 - RNA, 21 nt, Arabidopsis thaliana
ggguuuacagggagccaggca

SEQ ID NO: 139 - RNA, 21 nt, Arabidopsis thaliana
aggcauacagggagccaggca

SEQ ID NO: 140 - RNA, 21 nt, Arabidopsis thaliana
aggcauacagggagccaggca

SEQ ID NO: 141 - RNA, 21 nt, Arabidopsis thaliana
aggcauacagggagccaggca

SEQ ID NO: 142 - RNA, 21 nt, Arabidopsis thaliana
aggcauacagggagccaggca

SEQ ID NO: 143 - protein, 8 aa, Arabidopsis thaliana
AGMQGARQ

SEQ ID NO: 144 - protein, 8 aa, Arabidopsis thaliana
AGIQGARQ

SEQ ID NO: 145 - protein, 8 aa, Arabidopsis thaliana
VGLQGARH

SEQ ID NO: 146 - protein, 8 aa, Arabidopsis thaliana
AGIQGARH

SEQ ID NO: 147 - protein, 8 aa, Arabidopsis thaliana
AGIQGARH

SEQ ID NO: 148 - protein, 8 aa, Arabidopsis thaliana
AGIQGARH

SEQ ID NO: 149 - protein, 8 aa, Arabidopsis thaliana
AGIQGARH

SEQ ID NO: 150 - RNA, 21 nt, Arabidopsis thaliana
ugcacgugcccugcuucucca

SEQ ID NO: 151 - RNA, 21 nt, Arabidopsis thaliana
agcacguacccugcuucucca

SEQ ID NO: 152 - RNA, 21 nt, Arabidopsis thaliana
uuuacgugcccugcuucucca

SEQ ID NO: 153 - RNA, 21 nt, Arabidopsis thaliana
ucuacgugcccugcuucucca

SEQ ID NO: 154 - RNA, 21 nt, Arabidopsis thaliana
agcacguguccuguuucucca

SEQ ID NO: 155 - RNA, 21 nt, Arabidopsis thaliana
agcacguguccuguuucucca

SEQ ID NO: 156 - RNA, 21 nt, Arabidopsis thaliana
cgcacgugaccugcuucucca

SEQ ID NO: 157 - protein, 8 aa, Arabidopsis thaliana
EHVPCFSN

SEQ ID NO: 158 - protein, 8 aa, Arabidopsis thaliana
VYVPCFSN

SEQ ID NO: 159 - protein, 8 aa, Arabidopsis thaliana
VYVPCFSN

SEQ ID NO: 160 - protein, 8 aa, Arabidopsis thaliana
EHVSCFSN

SEQ ID NO: 161 - protein, 8 aa, Arabidopsis thaliana
EHVSCFST

SEQ ID NO: 162 - protein, 8 aa, Arabidopsis thaliana
AHVTCFSN

SEQ ID NO: 163 - RNA, 21 nt, Arabidopsis thaliana
uagaucaugcuggcagcuuca

SEQ ID NO: 164 - RNA, 21 nt, Arabidopsis thaliana
uagaucaggcuggcagcuugu

SEQ ID NO: 165 - RNA, 21 nt, Arabidopsis thaliana
uagaucaggcuggcagcuugu

SEQ ID NO: 166 - protein, 8 aa, Arabidopsis thaliana
LRSGWQLV

SEQ ID NO: 167 - protein, 8 aa, Arabidopsis thaliana
DRSGWQLV

SEQ ID NO: 168 - RNA, 21 nt, Arabidopsis thaliana
ucggcaagucauccuuggcug

SEQ ID NO: 169 - RNA, 21 nt, Arabidopsis thaliana
aagggaagucauccuuggcug

SEQ ID NO: 170 - RNA, 21 nt, Arabidopsis thaliana
acgggaagucauccuuggcua

SEQ ID NO: 171 - RNA, 21 nt, Arabidopsis thaliana
uaggcaacucauucuuggcug

SEQ ID NO: 172 - RNA, 21 nt, Arabidopsis thaliana
uaggcaauucauccuuggcuu

SEQ ID NO: 173 - RNA, 21 nt, Arabidopsis thaliana
gauauuggcgcggcucaauca

SEQ ID NO: 174 - RNA, 21 nt, Arabidopsis thaliana
gauauuggcgcggcucaauca

SEQ ID NO: 175 - RNA, 21 nt, Arabidopsis thaliana
gauauuggcgcggcucaauca

SEQ ID NO: 176 - RNA, 21 nt, Arabidopsis thaliana
gauauuggcgcggcucaauca

SEQ ID NO: 177 - RNA, 21 nt, Arabidopsis thaliana
gauauuggcgcggcucaauca

SEQ ID NO: 178 - RNA, 21 nt, Arabidopsis thaliana
gauauuggcgcggcucaauca

SEQ ID NO: 179 - RNA, 21 nt, Arabidopsis thaliana
gauauuggcgcggcucaauca

SEQ ID NO: 180 - RNA, 21 nt, Arabidopsis thaliana
gauauuggcgcggcucaauca

SEQ ID NO: 181 - RNA, 21 nt, Arabidopsis thaliana
gauauuggcgcggcucaauua

SEQ ID NO: 182 - protein, 8 aa, Arabidopsis thaliana
GILARLNH

SEQ ID NO: 183 - protein, 8 aa, Arabidopsis thaliana
GILARLNH

SEQ ID NO: 184 - protein, 8 aa, Arabidopsis thaliana
GILARLNQ

SEQ ID NO: 185 - protein, 8 aa, Arabidopsis thaliana
EILARLNQ

SEQ ID NO: 186 - protein, 8 aa, Arabidopsis thaliana
EILARLNH

SEQ ID NO: 187 - protein, 8 aa, Arabidopsis thaliana
EILARLNQ

SEQ ID NO: 188 - protein, 8 aa, Arabidopsis thaliana
EILARLNQ

SEQ ID NO: 189 - protein, 8 aa, Arabidopsis thaliana
EILARLNY

SEQ ID NO: 190 - RNA, 21 nt, Arabidopsis thaliana
uugggaugaagccugguccgg

SEQ ID NO: 191 - RNA, 21 nt, Arabidopsis thaliana
ucggaccaggcuucauucccc

SEQ ID NO: 192 - RNA, 21 nt, Arabidopsis thaliana
ucggaccaggcuucauccccc

SEQ ID NO: 193 - RNA, 21 nt, Arabidopsis thaliana
ccggaccaggcuucaucccaa

SEQ ID NO: 194 - RNA, 21 nt, Arabidopsis thaliana
ccggaucaggcuucaucccaa

SEQ ID NO: 195 - RNA, 21 nt, Arabidopsis thaliana
uugggaugaagccugauccgg

SEQ ID NO: 196 - DNA, 27 bp, artificial sequence
  oligonucleotide
gatccattcctaagcgaagtttcagag

SEQ ID NO: 197 - DNA, 25 bp, artificial sequence
  oligonucleotide
gcccgagcaacataaagatccatag

SEQ ID NO: 198 - DNA, 22 bp, artificial sequence
  oligonucleotide
agaccttggcggagttcctttg

SEQ ID NO: 199 - DNA, 24 bp, artificial sequence
  oligonucleotide
gttgcgtgaaacagctacgatacc

SEQ ID NO: 200 - DNA, 23 bp, artificial sequence
  oligonucleotide
tctttccctgctcaatgctcctc

SEQ ID NO: 201 - DNA, 24 bp, artificial sequence
  oligonucleotide
tttcgccactgtctctcctctaac

SEQ ID NO: 202 - DNA, 22 bp, artificial sequence
  oligonucleotide
ctggaggttttgaggctggtat

SEQ ID NO: 203 - DNA, 21 bp, artificial sequence
  oligonucleotide
ccaagggtgaaagcaagaaga

SEQ ID NO: 204 - DNA, 56 bp, artificial sequence
  oligonucleotide
ctcgactctcgaggtagtattaattaacgagttctaagttcttcttccgttatgag

SEQ ID NO: 205 - DNA, 56 bp, artificial sequence
  oligonucleotide
ggttctggtacctgggtaggactcacctcagacagtgtaggctgagaagacaccgc

SEQ ID NO: 206 - DNA, 60 bp, artificial sequence
  oligonucleotide
ccaccgcagagacaatcagtgccggagctccatcaggctacctcacctacttatcaagcg

SEQ ID NO: 207 - DNA, 60 bp, artificial sequence
  oligonucleotide
cgcttgataagtaggtgaggtagcctgatggagctccggcactgattgtctctgcggtgg

SEQ ID NO: 208 - DNA, 58 bp, artificial sequence
  oligonucleotide
caccatcgggctcggattcgcctggtggaggtccggcaccaattcggctgacacagcc

SEQ ID NO: 209 - DNA, 58 bp, artificial sequence
  oligonucleotide
ggctgtgtcagccgaattggtgccggacctccaccaggcgaatccgagcccgatggtg

SEQ ID NO: 210 - DNA, 62 bp, artificial sequence
  oligonucleotide
ttggtttgtgagcagggattggagccggccttccatcagctgaatcggatcctcgaggtgta

SEQ ID NO: 211 - DNA, 62 bp, artificial sequence
  oligonucleotide
tacacctcgaggatccgattcagctgatggaaggccggctccaatccctgctcacaaaccaa

SEQ ID NO: 212 - RNA, 24 nt, Arabidopsis thaliana
guucccgagcugcaucaagcuacc

SEQ ID NO: 213 - protein, 8 aa, Arabidopsis thaliana
VPELHQAT

SEQ ID NO: 214 - RNA, 21 nt, Arabidopsis thaliana
ucgcuuggugcaggucgggaa

SEQ ID NO: 215 - RNA, 24 nt, Arabidopsis thaliana
guucccgagcuccaucaggcuacc

SEQ ID NO: 216 - protein, 8 aa, Arabidopsis thaliana
VPELHQAT

SEQ ID NO: 217 - RNA, 21 nt, Arabidopsis thaliana
ucgcuuggugcaggucgggaa

SEQ ID NO: 218 - RNA, 24 nt, Arabidopsis thaliana
gugccggagcuccaucaggcuacc

SEQ ID NO: 219 - protein, 8 aa, Arabidopsis thaliana
VPELHQAT

SEQ ID NO: 220 - RNA, 21 nt, Arabidopsis thaliana
ucgcuuggugcaggucgggaa

SEQ ID NO: 221 - RNA, 24 nt, Arabidopsis thaliana
guucccgagcugcaucaagcuacc

SEQ ID NO: 222 - RNA, 21 nt, Arabidopsis thaliana
aagggcuggacgugguucccu

SEQ ID NO: 223 - RNA, 21 nt, Arabidopsis thaliana
ucgcuuggugcaggucgggaa

SEQ ID NO: 224 - RNA, 24 nt, Arabidopsis thaliana
gaucccgccuugcaucaacugaau

SEQ ID NO: 225 - RNA, 24 nt, Arabidopsis thaliana
gugccggagcuccaucaggcuacc

SEQ ID NO: 226 - RNA, 21 nt, Arabidopsis thaliana
ucgcuuggugcaggucgggaa

SEQ ID NO: 227 - RNA, 24 nt, Arabidopsis thaliana
gugccggagcuccaucaggcuacc

SEQ ID NO: 228 - RNA, 20 nt, Arabidopsis thaliana
cgccugguggagguccggca

SEQ ID NO: 229 - RNA, 24 nt, Arabidopsis thaliana
auucgccugguggagguccggcag

SEQ ID NO: 230 - RNA, 24 nt, Arabidopsis thaliana
gagccggccuuccaucagcugaau

SEQ ID NO: 231 - RNA, 21 nt, Arabidopsis thaliana
ugacagaagagagugagcaca

SEQ ID NO: 232 - RNA, 21 nt, Arabidopsis thaliana
uugacagaagauagagagcac

SEQ ID NO: 233 - RNA, 20 nt, Arabidopsis thaliana
ucccaaauguagacaaagca

SEQ ID NO: 234 - RNA, 21 nt, Arabidopsis thaliana
uuuggauugaagggagcucua

SEQ ID NO: 235 - RNA, 21 nt, Arabidopsis thaliana
uuuggauugaagggagcucuu

SEQ ID NO: 236 - RNA, 21 nt, Arabidopsis thaliana
uuuggauugaagggagcuccu

SEQ ID NO: 237 - RNA, 21 nt, Arabidopsis thaliana
ugccuggcucccuguaugcca

SEQ ID NO: 238 - RNA, 21 nt, Arabidopsis thaliana
uugaaagugacuacaucgggg

SEQ ID NO: 239 - RNA, 21 nt, Arabidopsis thaliana
ucgauaaaccucugcauccag

SEQ ID NO: 240 - RNA, 24 nt, Arabidopsis thaliana
uugaagaggacuuggaacuucgau

SEQ ID NO: 241 - RNA, 21 nt, Arabidopsis thaliana
uggagaagcagggcacgugca

SEQ ID NO: 242 - RNA, 21 nt, Arabidopsis thaliana
ucggaccaggcuucauccccc

SEQ ID NO: 243 - RNA, 21 nt, Arabidopsis thaliana
ucggaccaggcuucauucccc

SEQ ID NO: 244 - RNA, 21 nt, Arabidopsis thaliana
ugaagcugccagcaugaucua

SEQ ID NO: 245 - RNA, 21 nt, Arabidopsis thaliana
ugaagcugccagcaugaucug

SEQ ID NO: 246 - RNA, 21 nt, Arabidopsis thaliana
ucgcuuggugcaggucgggaa

SEQ ID NO: 247 - RNA, 21 nt, Arabidopsis thaliana
cagccaaggaugacuugccga

SEQ ID NO: 248 - RNA, 21 nt, Arabidopsis thaliana
ugauugagccgugucaauauc

SEQ ID NO: 249 - RNA, 21 nt, Arabidopsis thaliana
ugauugagccgcgccaauauc

SEQ ID NO: 250 - RNA, 21 nt, Arabidopsis thaliana
agaaucuugaugaugcugcau

SEQ ID NO: 251 - RNA, 21 nt, Arabidopsis thaliana
agaaucuugaugaugcugcag

SEQ ID NO: 252 - RNA, 21 nt, Arabidopsis thaliana
ggaaucuugaugaugcugcau

SEQ ID NO: 253 - RNA, 22 nt, Arabidopsis thaliana
uucgcuugcagagagaaaucac

SEQ ID NO: 254 - RNA, 20 nt, Arabidopsis thaliana
uuggacugaagggagcuccc

SEQ ID NO: 255 - RNA, 21 nt, Arabidopsis thaliana
uccaaagggaucgcauugauc

SEQ ID NO: 256 - RNA, 21 nt, Arabidopsis thaliana
uccaaagggaucgcauugauc

SEQ ID NO: 257 - RNA, 21 nt, Arabidopsis thaliana
uucuuuggcauucuguccacc

SEQ ID NO: 258 - RNA, 21 nt, Arabidopsis thaliana
uucuuuggcauucuguccacc

SEQ ID NO: 259 - RNA, 21 nt, Arabidopsis thaliana
cugaaguguuugggggaacuc

SEQ ID NO: 260 - RNA, 21 nt, Arabidopsis thaliana
cugaaguguuugggggaacuc

SEQ ID NO: 261 - RNA, 21 nt, Arabidopsis thaliana
cugaaguguuugggggaacuc

SEQ ID NO: 262 - RNA, 21 nt, Arabidopsis thaliana
cugaaguguuuggggggacuc

SEQ ID NO: 263 - RNA, 21 nt, Arabidopsis thaliana
cugaaguguuuggggggacuc

SEQ ID NO: 264 - RNA, 21 nt, Arabidopsis thaliana
cugaaguguuuggggggacuc

SEQ ID NO: 265 - RNA, 21 nt, Arabidopsis thaliana
uuccacagcuuucuugaacug

SEQ ID NO: 266 - RNA, 21 nt, Arabidopsis thaliana
uuccacagcuuucuugaacuu

SEQ ID NO: 267 - RNA, 21 nt, Arabidopsis thaliana
ucauugagugcagcguugaug

SEQ ID NO: 268 - RNA, 21 nt, Arabidopsis thaliana
ucauugagugcaucguugaug

SEQ ID NO: 269 - RNA, 21 nt, Arabidopsis thaliana
uguguucucaggucaccccuu

SEQ ID NO: 270 - RNA, 21 nt, Arabidopsis thaliana
uguguucucaggucaccccug

SEQ ID NO: 271 - RNA, 21 nt, Arabidopsis thaliana
uguguucucaggucaccccug

SEQ ID NO: 272 - RNA, 21 nt, Arabidopsis thaliana
ugccaaaggagauuugcccug

SEQ ID NO: 273 - RNA, 23 nt, Arabidopsis thaliana
ccugccaaaggagaguugcccug

SEQ ID NO: 274 - RNA, 23 nt, Arabidopsis thaliana
ccugccaaaggagaguugcccug

SEQ ID NO: 275 - RNA, 21 nt, Arabidopsis thaliana
ugccaaaggagauuugccccg

SEQ ID NO: 276 - RNA, 21 nt, Arabidopsis thaliana
ugccaaaggagauuugccucg

SEQ ID NO: 277 - RNA, 21 nt, Arabidopsis thaliana
ugccaaaggagauuugcccgg

SEQ ID NO: 278 - RNA, 21 nt, Arabidopsis thaliana
ugacagaagagagugagcaca

SEQ ID NO: 279 - RNA, 21 nt, Arabidopsis thaliana
ugacagaagagagugagcaca

SEQ ID NO: 280 - RNA, 21 nt, Arabidopsis thaliana
ugacagaagagagugagcaca

SEQ ID NO: 281 - RNA, 21 nt, Arabidopsis thaliana
ugacagaagagagugagcaca

SEQ ID NO: 282 - RNA, 21 nt, Arabidopsis thaliana
ugacagaagagagugagcaca

SEQ ID NO: 283 - RNA, 21 nt, Arabidopsis thaliana
ugacagaagagagugagcaca

SEQ ID NO: 284 - RNA, 21 nt, Arabidopsis thaliana
uugacagaagaaagagagcac

SEQ ID NO: 285 - RNA, 21 nt, Arabidopsis thaliana
cgacagaagagagugagcaca

SEQ ID NO: 286 - RNA, 21 nt, Arabidopsis thaliana
uugacagaagauagagagcac

SEQ ID NO: 287 - RNA, 21 nt, Arabidopsis thaliana
uugacagaagauagagagcac

SEQ ID NO: 288 - RNA, 21 nt, Arabidopsis thaliana
uugacagaagauagagagcac

SEQ ID NO: 289 - RNA, 21 nt, Arabidopsis thaliana
cugacagaagauagagagcac

SEQ ID NO: 290 - RNA, 20 nt, Arabidopsis thaliana
ucccaaauguagacaaagca

SEQ ID NO: 291 - RNA, 20 nt, Arabidopsis thaliana
ccccaaauguagacaaagca

SEQ ID NO: 292 - RNA, 21 nt, Arabidopsis thaliana
uuuggauugaagggagcucua

SEQ ID NO: 293 - RNA, 21 nt, Arabidopsis thaliana
uuuggauugaagggagcucuu

SEQ ID NO: 294 - RNA, 21 nt, Arabidopsis thaliana
uuuggauugaagggagcuccu

SEQ ID NO: 295 - RNA, 20 nt, Arabidopsis thaliana
uuggacugaagggagcuccc

SEQ ID NO: 296 - RNA, 20 nt, Arabidopsis thaliana
uuggacugaagggagcuccc

SEQ ID NO: 297 - RNA, 20 nt, Arabidopsis thaliana
uuggacugaagggagcuccu

SEQ ID NO: 298 - RNA, 21 nt, Arabidopsis thaliana
ugccuggcucccuguaugcca

SEQ ID NO: 299 - RNA, 21 nt, Arabidopsis thaliana
ugccuggcucccuguaugcca

SEQ ID NO: 300 - RNA, 21 nt, Arabidopsis thaliana
ugccuggcucccuguaugcca

SEQ ID NO: 301 - RNA, 21 nt, Arabidopsis thaliana
uugaaagugacuacaucgggg

SEQ ID NO: 302 - RNA, 21 nt, Arabidopsis thaliana
ucgauaaaccucugcauccag

SEQ ID NO: 303 - RNA, 21 nt, Arabidopsis thaliana
ucgauaaaccucugcauccag

SEQ ID NO: 304 - RNA, 24 nt, Arabidopsis thaliana
uugaagaggacuuggaacuucgau

SEQ ID NO: 305 - RNA, 21 nt, Arabidopsis thaliana
uggagaagcagggcacgugca

SEQ ID NO: 306 - RNA, 21 nt, Arabidopsis thaliana
uggagaagcagggcacgugca

SEQ ID NO: 307 - RNA, 21 nt, Arabidopsis thaliana
uggagaagcagggcacgugcg

SEQ ID NO: 308 - RNA, 21 nt, Arabidopsis thaliana
ucggaccaggcuucauccccc

SEQ ID NO: 309 - RNA, 21 nt, Arabidopsis thaliana
ucggaccaggcuucauccccc

SEQ ID NO: 310 - RNA, 21 nt, Arabidopsis thaliana
ucggaccaggcuucauucccc

SEQ ID NO: 311 - RNA, 21 nt, Arabidopsis thaliana
ucggaccaggcuucauucccc

SEQ ID NO: 312 - RNA, 21 nt, Arabidopsis thaliana
ucggaccaggcuucauucccc

SEQ ID NO: 313 - RNA, 21 nt, Arabidopsis thaliana
ucggaccaggcuucauucccc

SEQ ID NO: 314 - RNA, 21 nt, Arabidopsis thaliana
ucggaccaggcuucauucccc

SEQ ID NO: 315 - RNA, 21 nt, Arabidopsis thaliana
ucggaccaggcuucauucccc

SEQ ID NO: 316 - RNA, 21 nt, Arabidopsis thaliana
ucggaccaggcuucauucccc

SEQ ID NO: 317 - RNA, 21 nt, Arabidopsis thaliana
ugaagcugccagcaugaucua

SEQ ID NO: 318 - RNA, 21 nt, Arabidopsis thaliana
ugaagcugccagcaugaucua

SEQ ID NO: 319 - RNA, 21 nt, Arabidopsis thaliana
uuaagcugccagcaugaucuu

SEQ ID NO: 320 - RNA, 21 nt, Arabidopsis thaliana
ugaagcugccagcaugaucug

SEQ ID NO: 321 - RNA, 21 nt, Arabidopsis thaliana
ucgcuuggugcaggucgggaa

SEQ ID NO: 322 - RNA, 21 nt, Arabidopsis thaliana
ucgcuuggugcaggucgggaa

SEQ ID NO: 323 - RNA, 21 nt, Arabidopsis thaliana
cagccaaggaugacuugccga

SEQ ID NO: 324 - RNA, 21 nt, Arabidopsis thaliana
cagccaaggaugacuugccgg

SEQ ID NO: 325 - RNA, 21 nt, Arabidopsis thaliana
cagccaaggaugacuugccgg

SEQ ID NO: 326 - RNA, 21 nt, Arabidopsis thaliana
gagccaaggaugacuugccga

SEQ ID NO: 327 - RNA, 21 nt, Arabidopsis thaliana
gagccaaggaugacuugccgg

SEQ ID NO: 328 - RNA, 21 nt, Arabidopsis thaliana
gagccaaggaugacuugccga

SEQ ID NO: 329 - RNA, 21 nt, Arabidopsis thaliana
gagccaaggaugacuugccgg

SEQ ID NO: 330 - RNA, 21 nt, Arabidopsis thaliana
uagccaaggaugacuugccug

SEQ ID NO: 331 - RNA, 21 nt, Arabidopsis thaliana
uagccaaggaugacuugccug

SEQ ID NO: 332 - RNA, 21 nt, Arabidopsis thaliana
uagccaaggaugacuugccug

SEQ ID NO: 333 - RNA, 21 nt, Arabidopsis thaliana
uagccaaggaugacuugccug

SEQ ID NO: 334 - RNA, 21 nt, Arabidopsis thaliana
uagccaaggaugacuugccug

SEQ ID NO: 335 - RNA, 21 nt, Arabidopsis thaliana
uagccaaggaugacuugccug

SEQ ID NO: 336 - RNA, 21 nt, Arabidopsis thaliana
uagccaaggaugacuugccug

SEQ ID NO: 337 - RNA, 21 nt, Arabidopsis thaliana
ugauugagccgugucaauauc

SEQ ID NO: 338 - RNA, 21 nt, Arabidopsis thaliana
ugauugagccgcgccaauauc

SEQ ID NO: 339 - RNA, 21 nt, Arabidopsis thaliana
cgauugagccgugccaauauc

SEQ ID NO: 340 - RNA, 21 nt, Arabidopsis thaliana
ugauugagccgugccaauauc

SEQ ID NO: 341 - RNA, 21 nt, Arabidopsis thaliana
agaaucuugaugaugcugcau

SEQ ID NO: 342 - RNA, 21 nt, Arabidopsis thaliana
agaaucuugaugaugcugcau

SEQ ID NO: 343 - RNA, 21 nt, Arabidopsis thaliana
agaaucuugaugaugcugcag

SEQ ID NO: 344 - RNA, 21 nt, Arabidopsis thaliana
agaaucuugaugaugcugcag

SEQ ID NO: 345 - RNA, 21 nt, Arabidopsis thaliana
ggaaucuugaugaugcugcau

SEQ ID NO: 346 - RNA, 22 nt, Arabidopsis thaliana
uucgcuugcagagagaaaucac

SEQ ID NO: 347 - RNA, 21 nt, Arabidopsis thaliana
uccaaagggaucgcauugauc

SEQ ID NO: 348 - RNA, 21 nt, Arabidopsis thaliana
uccaaagggaucgcauugauc

SEQ ID NO: 349 - RNA, 20 nt, Arabidopsis thaliana
uuggcauucuguccaccucc

SEQ ID NO: 350 - RNA, 20 nt, Arabidopsis thaliana
uuggcauucuguccaccucc

SEQ ID NO: 351 - RNA, 21 nt, Arabidopsis thaliana
cugaaguguuugggggaacuc

SEQ ID NO: 352 - RNA, 21 nt, Arabidopsis thaliana
cugaaguguuuggggggacuc

SEQ ID NO: 353 - RNA, 21 nt, Arabidopsis thaliana
cugaaguguuuggggggacuc

SEQ ID NO: 354 - RNA, 21 nt, Arabidopsis thaliana
cugaaguguuugggggaacuc

SEQ ID NO: 355 - RNA, 21 nt, Arabidopsis thaliana
cugaaguguuugggggaacuc

SEQ ID NO: 356 - RNA, 21 nt, Arabidopsis thaliana
cugaaguguuuggggggacuc

SEQ ID NO: 357 - RNA, 21 nt, Arabidopsis thaliana
uuccacagcuuucuugaacug

SEQ ID NO: 358 - RNA, 21 nt, Arabidopsis thaliana
uuccacagcuuucuugaacuu

SEQ ID NO: 359 - RNA, 21 nt, Arabidopsis thaliana
ucauugagugcagcguugaug

SEQ ID NO: 360 - RNA, 21 nt, Arabidopsis thaliana
ucauugagugcaucguugaug

SEQ ID NO: 361 - RNA, 21 nt, Arabidopsis thaliana
uguguucucaggucaccccuu

SEQ ID NO: 362 - RNA, 21 nt, Arabidopsis thaliana
uguguucucaggucaccccug

SEQ ID NO: 363 - RNA, 21 nt, Arabidopsis thaliana
uguguucucaggucaccccug

SEQ ID NO: 364 - RNA, 21 nt, Arabidopsis thaliana
ugccaaaggagauuugcccug

SEQ ID NO: 365 - RNA, 21 nt, Arabidopsis thaliana
ugccaaaggagaguugcccug

SEQ ID NO: 366 - RNA, 21 nt, Arabidopsis thaliana
ugccaaaggagaguugcccug

SEQ ID NO: 367 - RNA, 21 nt, Arabidopsis thaliana
ugccaaaggagauuugccccg

SEQ ID NO: 368 - RNA, 21 nt, Arabidopsis thaliana
ugccaaaggagauuugccucg

SEQ ID NO: 369 - RNA, 21 nt, Arabidopsis thaliana
ugccaaaggagauuugcccgg

SEQ ID NO: 370 - RNA, 27 nt, Arabidopsis thaliana
aacugacagaagagagugagcacacaa

SEQ ID NO: 371 - RNA, 27 nt, Arabidopsis thaliana
aacugacagaagagagugagcacaugc

SEQ ID NO: 372 - RNA, 27 nt, Arabidopsis thaliana
aacugacagaagagagugagcacacaa

SEQ ID NO: 373 - RNA, 28 nt, Arabidopsis thaliana
ugacagaagagagugagcacacaaaggg

SEQ ID NO: 374 - RNA, 31 nt, Arabidopsis thaliana
ggugacagaagagagugagcacacauggugg

SEQ ID NO: 375 - RNA, 32 nt, Arabidopsis thaliana
uggugacagaagagagugagcacacauggugg

SEQ ID NO: 376 - RNA, 21 nt, Arabidopsis thaliana
uugacagaagaaagagagcac

SEQ ID NO: 377 - RNA, 30 nt, Arabidopsis thaliana
ggcgacagaagagagugagcacacauggcu

SEQ ID NO: 378 - RNA, 23 nt, Arabidopsis thaliana
guugacagaagauagagagcaca

SEQ ID NO: 379 - RNA, 24 nt, Arabidopsis thaliana
uguugacagaagauagagagcaca

SEQ ID NO: 380 - RNA, 24 nt, Arabidopsis thaliana
uuguugacagaagauagagagcac

SEQ ID NO: 381 - RNA, 20 nt, Arabidopsis thaliana
ugacagaagauagagagcac

SEQ ID NO: 382 - RNA, 24 nt, Arabidopsis thaliana
cguuuggauugaagggagcuccuu

SEQ ID NO: 383 - RNA, 24 nt, Arabidopsis thaliana
uugauuggacugaagggagcuccc

SEQ ID NO: 384 - RNA, 26 nt, Arabidopsis thaliana
cuaugcuuggacugaagggagcuccc

SEQ ID NO: 385 - RNA, 23 nt, Arabidopsis thaliana
gccuggcucccuguaugccauau

SEQ ID NO: 386 - RNA, 29 nt, Arabidopsis thaliana
gucgugccuggcucccuguaugccacaag

SEQ ID NO: 387 - RNA, 30 nt, Arabidopsis thaliana
cguuaugccuggcucccuguaugccacgag

SEQ ID NO: 388 - RNA, 30 nt, Arabidopsis thaliana
ugaauagaucgauaaaccucugcauccagc

SEQ ID NO: 389 - RNA, 29 nt, Arabidopsis thaliana
gaaucgaucgauaaaccucugcauccagc

SEQ ID NO: 390 - RNA, 30 nt, Arabidopsis thaliana
ccauguuggagaagcagggcacgugcaaac

SEQ ID NO: 391 - RNA, 25 nt, Arabidopsis thaliana
aagauggagaagcagggcacgugca

SEQ ID NO: 392 - RNA, 27 nt, Arabidopsis thaliana
cuugauggagaagcagggcacgugcga

SEQ ID NO: 393 - RNA, 27 nt, Arabidopsis thaliana
guauccucggaccaggcuucauccccc

SEQ ID NO: 394 - RNA, 21 nt, Arabidopsis thaliana
ucggaccaggcuucauccccc

SEQ ID NO: 395 - RNA, 21 nt, Arabidopsis thaliana
ucggaccaggcuucauucccc

SEQ ID NO: 396 - RNA, 21 nt, Arabidopsis thaliana
ucggaccaggcuucauucccc

SEQ ID NO: 397 - RNA, 21 nt, Arabidopsis thaliana
ucggaccaggcuucauucccc

SEQ ID NO: 398 - RNA, 25 nt, Arabidopsis thaliana
ucggaccaggcuucauuccccucaa

SEQ ID NO: 399 - RNA, 27 nt, Arabidopsis thaliana
ucggaccaggcuucauuccccucaacu

SEQ ID NO: 400 - RNA, 26 nt, Arabidopsis thaliana
ucggaccaggcuucauuccccucaac

SEQ ID NO: 401 - RNA, 29 nt, Arabidopsis thaliana
cugaugaagcugccagcaugaucuaauua

SEQ ID NO: 402 - RNA, 30 nt, Arabidopsis thaliana
aagugaagcugccagcaugaucuaucuuug

SEQ ID NO: 403 - RNA, 27 nt, Arabidopsis thaliana
caguuaagcugccagcaugaucuuguc

SEQ ID NO: 404 - RNA, 30 nt, Arabidopsis thaliana
cucggauucgcuuggugcaggucgggaacc

SEQ ID NO: 405 - RNA, 29 nt, Arabidopsis thaliana
cucggauucgcuuggugcaggucgggaac

SEQ ID NO: 406 - RNA, 29 nt, Arabidopsis thaliana
agugugcagccaaggaugacuugccgauu

SEQ ID NO: 407 - RNA, 30 nt, Arabidopsis thaliana
auaaugcagccaaggaugacuugccggaac

SEQ ID NO: 408 - RNA, 26 nt, Arabidopsis thaliana
guucagccaaggaugacuugccggua

SEQ ID NO: 409 - RNA, 26 nt, Arabidopsis thaliana
gauugagccaaggaugacuugccgau

SEQ ID NO: 410 - RNA, 25 nt, Arabidopsis thaliana
uugagccaaggaugacuugccgguu

SEQ ID NO: 411 - RNA, 26 nt, Arabidopsis thaliana
gauugagccaaggaugacuugccgau

SEQ ID NO: 412 - RNA, 28 nt, Arabidopsis thaliana
gguugagccaaggaugacuugccggguu

SEQ ID NO: 413 - RNA, 22 nt, Arabidopsis thaliana
gugugguagccaaggaugacuu

SEQ ID NO: 414 - RNA, 22 nt, Arabidopsis thaliana
auuugguagccaaggaugacuu

SEQ ID NO: 415 - RNA, 21 nt, Arabidopsis thaliana
uuuaguagccaaggaugacuu

SEQ ID NO: 416 - RNA, 22 nt, Arabidopsis thaliana
auuugguagccaaggaugacuu

SEQ ID NO: 417 - RNA, 20 nt, Arabidopsis thaliana
uuaauagccaaggaugacuu

SEQ ID NO: 418 - RNA, 22 nt, Arabidopsis thaliana
auuugguagccaaggaugacuu

SEQ ID NO: 419 - RNA, 21 nt, Arabidopsis thaliana
uuuaguagccaaggaugacuu

SEQ ID NO: 420 - RNA, 23 nt, Arabidopsis thaliana
ugauugagccgugucaauaucuc

SEQ ID NO: 421 - RNA, 31 nt, Arabidopsis thaliana
uuaucugauugagccgcgccaauaucucagu

SEQ ID NO: 422 - RNA, 30 nt, Arabidopsis thaliana
uguucgauugagccgugccaauaucacgcg

SEQ ID NO: 423 - RNA, 28 nt, Arabidopsis thaliana
uuauuugauugagccgugccaauaucac

SEQ ID NO: 424 - RNA, 30 nt, Arabidopsis thaliana
aaugagaaucuugaugaugcugcaucggca

SEQ ID NO: 425 - RNA, 26 nt, Arabidopsis thaliana
uaugagaaucuugaugaugcugcauc

SEQ ID NO: 426 - RNA, 31 nt, Arabidopsis thaliana
uaugagaaucuugaugaugcugcagcugcaa

SEQ ID NO: 427 - RNA, 32 nt, Arabidopsis thaliana
guuugagaaucuugaugaugcugcagcggcaa

SEQ ID NO: 428 - RNA, 21 nt, Arabidopsis thaliana
gaaucuugaugaugcugcauc

SEQ ID NO: 429 - RNA, 34 nt, Arabidopsis thaliana
gaggaaggauccaaagggaucgcauugauccuaa

SEQ ID NO: 430 - RNA, 30 nt, Arabidopsis thaliana
gaaaggauccaaagggaucgcauugauccu

SEQ ID NO: 431 - RNA, 27 nt, Arabidopsis thaliana
aucuuuggcauucuguccaccuccuuc

SEQ ID NO: 432 - RNA, 32 nt, Arabidopsis thaliana
cagagaucuuuggcauucuguccaccuccucu

SEQ ID NO: 433 - RNA, 28 nt, Arabidopsis thaliana
cacugaaguguuugggggaacucccgga

SEQ ID NO: 434 - RNA, 25 nt, Arabidopsis thaliana
acugaaguguuuggggggacucuug

SEQ ID NO: 435 - RNA, 24 nt, Arabidopsis thaliana
acugaaguguuuggggggacucuu

SEQ ID NO: 436 - RNA, 27 nt, Arabidopsis thaliana
cacugaaguguuugggggaacucccga

SEQ ID NO: 437 - RNA, 26 nt, Arabidopsis thaliana
cuacugaaguguuugggggaacuccc

SEQ ID NO: 438 - RNA, 31 nt, Arabidopsis thaliana
acugaaguguuuggggggacucuaggugaca

SEQ ID NO: 439 - RNA, 20 nt, Arabidopsis thaliana
uuccacagcuuucuugaacu

SEQ ID NO: 440 - RNA, 30 nt, Arabidopsis thaliana
cauacuuuuccacagcuuucuugaacuuuc

SEQ ID NO: 441 - RNA, 26 nt, Arabidopsis thaliana
acaucauugagugcaucguugaugua

SEQ ID NO: 442 - RNA, 26 nt, Arabidopsis thaliana
uuguguucucaggucaccccuuugaa

SEQ ID NO: 443 - RNA, 26 nt, Arabidopsis thaliana
cauguguucucaggucaccccugcug

SEQ ID NO: 444 - RNA, 25 nt, Arabidopsis thaliana
auguguucucaggucaccccugcug

SEQ ID NO: 445 - RNA, 25 nt, Arabidopsis thaliana
aucugccaaaggagauuugcccugu

SEQ ID NO: 446 - RNA, 32 nt, Arabidopsis thaliana
accugccaaaggagaguugcccugaaacuggu

SEQ ID NO: 447 - RNA, 26 nt, Arabidopsis thaliana
cuugccaaaggagaguugcccuguca

SEQ ID NO: 448 - RNA, 31 nt, Arabidopsis thaliana
cucugccaaaggagauuugccccgcaauuca

SEQ ID NO: 449 - RNA, 27 nt, Arabidopsis thaliana
uccucugccaaaggagauuugccucgc

SEQ ID NO: 450 - RNA, 33 nt, Arabidopsis thaliana
ugagcucucugccaaaggagauuugcccgguaa

SEQ ID NO: 451 - RNA, 102 nt, Arabidopsis thaliana
gcaaagaaacugacagaagagagugagcacacaaaggcaauuugcauaucauugcacuugcuucucuugcgugcucacugcucuuucugucagauuccggug

SEQ ID NO: 452 - RNA, 100 nt, Arabidopsis thaliana
cagagaaaacugacagaagagagugagcacaugcaggcacuguuaugugucuauaacuuugcgugugcgugcucaccucucuuucugucaguugccuauc

SEQ ID NO: 453 - RNA, 103 nt, Arabidopsis thaliana
gcauagaaacugacagaagagagugagcacacaaaggcacuuugcauguucgaugcauuugcuucucuugcgugcucacugcucuaucugucagauuccggcu

SEQ ID NO: 454 - RNA, 106 nt, Arabidopsis thaliana
gaaaagaaguugacagaagagagugagcacacaaaggggaaguuguauaaaaguuuuguauaugguugcuuuugcgugcucacucucuuuuugucauaacuucucc

SEQ ID NO: 455 - RNA, 116 nt, Arabidopsis thaliana
aauuaggaggugacagaagagagugagcacacauggugguuucuugcaugcuuuuuugauuaggguuucaugcuugaagcuaugugugcuuacucucucucugucaccccuucucu

SEQ ID NO: 456 - RNA, 110 nt, Arabidopsis thaliana
gaauugauggugacagaagagagugagcacacaugguggcuuucuugcauauuugaagguuccaugcuugaagcuaugugugcucacucucuauccgucacccccuucuc

SEQ ID NO: 457 - RNA, 106 nt, Arabidopsis thaliana
augaaaaauguugacagaagaaagagagcacaaccugggauuagcaaaaagauaguuuugcccuugucgggagugugcucucuuuccuucugccaccaucauugcg

SEQ ID NO: 458 - RNA, 103 nt, Arabidopsis thaliana
auaacgaaggcgacagaagagagugagcacacauggcucuuuuucuagcaugcucaugcucgaaagcucugcgugcuuacucucuucuugucuccugcucucu

SEQ ID NO: 459 - RNA, 111 nt, Arabidopsis thaliana
auugauaguguugacagaagauagagagcacagaugaugagauacaauucggagcauguucuuugcaucuuacuccuuugugcucucuagccuucugucaucaccuuuuau

SEQ ID NO: 460 - RNA, 111 nt, Arabidopsis thaliana
auugauaguguugacagaagauagagagcacagaugauaagauacaauuccucgcagcuucuuugcaucuuacuccuuugugcucucuagccuucugucaucacccguuau

SEQ ID NO: 461 - RNA, 185 nt, Arabidopsis thaliana
auguugguuguugacagaagauagagagcacuaaggaugacaugcaaguacauacauauauaucaucacaccgcauguggaugauaaaauauguauaacaaauucaaagaaagagagggagagaaagagagagaaccugcaucucuacucuuuugugcucucuauacuucugucaccaccuuuau

SEQ ID NO: 462 - RNA, 192 nt, Arabidopsis thaliana
agugugguugcugacagaagauagagagcacuaaggaugcuaugcaaaacagacacagauauguguuucuaauuguauuucauacuuuaaccucaaaguugauauaaaaaaagaaagaaagauagaagagcuagaagacuaucugcaucucuauuccuaugugcucucuaugcuucugucaucaccuuucuu

SEQ ID NO: 463 - RNA, 85 nt, Arabidopsis thaliana
aucucugugcuucuuugucuacaauuuuggaaaaagugaugacgccauugcucuuucccaaauguagacaaagcaauaccgugau

SEQ ID NO: 464 - RNA, 85 nt, Arabidopsis thaliana
aucucugugcuucuuugucuacacuuuuggaaaaggugaugauaucauugcuuuuccccaaauguagacaaagcaauaccgugau

SEQ ID NO: 465 - RNA, 202 nt, Arabidopsis thaliana
acgauggaaguagagcuccuuaaaguucaaacaugaguugagcaggguaaagaaaagcugcuaagcuauggaucccauaagcccuaauccuuguaaaguaaaaaaggauuugguuauauggauugcauaucucaggagcuuuaacuugcccuuuaauggcuuuuacucuucuuuggauugaagggagcucuacaucuucuuu

SEQ ID NO: 466 - RNA, 206 nt, Arabidopsis thaliana
gaagaagaggaagagcuccuugaaguucaauggaggguuuagcagggugaaguaaagcugcuaagcuauggaucccauaagccuuaucaaauucaauauaauugaugauaagguuuuuuuuauggaugccauaucucaggagcuuucacuuaccccuuuaauggcuucacucuucuuuggauugaagggagcucuucaucucucca

SEQ ID NO: 467 - RNA, 225 nt, Arabidopsis thaliana
guguaacagaaggagcucccuuccuccaaaacgaagaggacaagauuugaggaacuaaaaugcagaaucuaagaguucaugucuuccucauagagagugcgcgguguuaaaagcuugaagaaagcacacuuuaaggggauugcacgaccucuuagauucucccucuuucucuacauaucauucucuucucuucguuuggauugaagggagcuccuuuucuucuuc

SEQ ID NO: 468 - RNA, 190 nt, Arabidopsis thaliana
uauauguagagagagcuuccuugaguccauucacaggucgugauaugauucaauuagcuuccgacucauucauccaaauaccgagucgccaaaauucaaacuagacucguuaaaugaaugaaugaugcgguagacaaauuggaucauugauucucuuugauuggacugaagggagcucccucucucuuuu

SEQ ID NO: 469 - RNA, 190 nt, Arabidopsis thaliana
gguggaggaagagagcuuucuucgguccacucauggaguaauaugugagauuuaauugacucucgacucauucauccaaauaccaaaugaaagaauuuguucucauaugguaaaugaaugaaugaugcgagagacaaauugagucuucacuucucuaugcuuggacugaagggagcucccuauuuuuauc

SEQ ID NO: 470 - RNA, 199 nt, Arabidopsis thaliana
uagauauagaaggagauucuuucaguccagucauggauagaaaaagaagaggguagaaauaucugccgacucauccauccaaacacucgugguagagaaacgauaaauuuaaaccgcagugacugugugaaugaugcgggagauauuuuugauccuucuuuaucuguguuuggacugaagggagcuccuucuuuuucua

SEQ ID NO: 471 - RNA, 99 nt, Arabidopsis thaliana
uauauauguaugccuggcucccuguaugccauaugcugagcccaucgaguaucgaugaccuccguggauggcguaugaggagccaugcauauccucaua

SEQ ID NO: 472 - RNA, 101 nt, Arabidopsis thaliana
auaauagucgugccuggcucccuguaugccacaagaaaacaucgauuuaguuucaaaaucgaucacuaguggcguacagaguagucaagcaugaccaaagc

SEQ ID NO: 473 - RNA, 101 nt, Arabidopsis thaliana
uuugucguuaugccuggcucccuguaugccacgaguggauaccgauuuugguuuuaaaaucggcugccgguggcguacaaggagucaagcaugaccagaag

SEQ ID NO: 474 - RNA, 110 nt, Arabidopsis thaliana
gaucaaugcauugaaagugacuacaucgggguuccgauuuuuuuuguucuucauaugaugaagcggaaacaguaaucaacccugguuuagucacuuucacugcauuaauc

SEQ ID NO: 475 - RNA, 105 nt, Arabidopsis thaliana
gugagagucgcuggaggcagcgguucaucgaucucuuccugugaacacauuaaaaauguaaaagcaugaauagaucgauaaaccucugcauccagcguuugccuc

SEQ ID NO: 476 - RNA, 108 nt, Arabidopsis thaliana
agugaagucgcuggaggcagcgguucaucgaucaauuccugugaauauuuauuuuuguuuacaaaagcaagaaucgaucgauaaaccucugcauccagcgcugcuugc

SEQ ID NO: 477 - RNA, 323 nt, Arabidopsis thaliana
gguggauaaaaucgaguuccaaccucuucaacgacaacgauuucaacacucucuuccaggaacaacuuccuccaggcagaugauacuaaagugcuggaguucccgguuccugagagugaguccauaucaaaaugcgcauucguuaucacuugguugaacccauuuggggauuuaaauuuggaggugaaauggaacgcguaauugaugacuccuacguggaaccucuucuuaggaagagcacggucgaagaaguaacugcgcagugcuuaaaucguagaugcuaaagucguugaagaggacuuggaacuucgauauuauccccc

SEQ ID NO: 478 - RNA, 98 nt, Arabidopsis thaliana
aucuccauguuggagaagcagggcacgugcaaaccaacaaacacgaaauccgucucauuugcuuauuugcacguacuuaacuucuccaacaugagcuc

SEQ ID NO: 479 - RNA, 169 nt, Arabidopsis thaliana
augagcaagauggagaagcagggcacgugcauuacuagcucauauauacacucucaccacaaaugcguguauauaugcggaauuuugugauauagauguguguguguguugagugugaugauauggaugaguuaguucuucaugugcccaucuucaccaucaugaccac

SEQ ID NO: 480 - RNA, 102 nt, Arabidopsis thaliana
uaacacuugauggagaagcagggcacgugcgaacacaaaugaaaucgaucgguacuuguugaucauauuuucgcacguguucuacuacuccaacacgugucu

SEQ ID NO: 481 - RNA, 121 nt, Arabidopsis thaliana
uuucaguugaggggaauguugucuggaucgaggauauuauagauauauacauguguauguuaaugauucaagugaucauagagaguauccucggaccaggcuucaucccccccaacauguu

SEQ ID NO: 482 - RNA, 156 nt, Arabidopsis thaliana
uuucuguuguggggaauguuguuuggaucgaggauaucauaaacgcauacacauguuuauauguuaugaugcauuauaugacugauguaauguacauauauauacauacaugccacaugguaucgucggaccaggcuucaucccccucaacauguu

SEQ ID NO: 483 - RNA, 156 nt, Arabidopsis thaliana
ucucuuuugaggggacuguugucuggcucgaggacucuggcucgcucuauucauguuggaucucuuucgaucuaacaaucgaauugaaccuucagauuucagauuugauuaggguuuuagcgucuucggaccaggcuucauuccccccaauuguug

SEQ ID NO: 484 - RNA, 132 nt, Arabidopsis thaliana
uuucuuuugaggggacuguugucuggcucgaggacucuuauucuaauacaaucucauuugaauacauucagaucugaugauugauuaggguuuuagugucgucggaccaggcuucauuccccccaauuauca

SEQ ID NO: 485 - RNA, 128 nt, Arabidopsis thaliana
uuaguguugagaggauuguugucuggcucgaggucaugaagaagagaaucacucgaauuaauuuggaagaacaaauuaagaaaacccuagaugauucucggaccaggcuucauucccccuaaccuacu

SEQ ID NO: 486 - RNA, 121 nt, Arabidopsis thaliana
uuaggguugagaggaauauugucuggcucgaggucaugaagaagaucgguagauugauucauuuuaaagagugaaaucccuaaaugauucucggaccaggcuucauuccccccaaccgaca

SEQ ID NO: 487 - RNA, 155 nt, Arabidopsis thaliana
uuccuuuugaggggaauguugucuggcacgaggcccuuaacuuagaucuauauuugauuauauauauaugucucuucuuuauucauuagucuauacaugaaugaucauuuuacgguuaaugacgucggaccaggcuucauuccccucaauuauau

SEQ ID NO: 488 - RNA, 111 nt, Arabidopsis thaliana
caaaaguucaggugaaugaugccuggcucgagaccauucaaucucaugaucucaugauuauaacgaugaugaugaugaugucggaccaggcuucauuccccucaacuuaca

SEQ ID NO: 489 - RNA, 110 nt, Arabidopsis thaliana
uuaggguuuagaggaauguuguuuggcucgaggucauggagaguaauucguuaacccaacucaaaacucuaaaugauucucggaccaggcuucauuccccucaaccuauu

SEQ ID NO: 490 - RNA, 121 nt, Arabidopsis thaliana
cggcaucugaugaagcugccagcaugaucuaauuagcuuucuuuauccuuuguuguguuucaugacgaugguuaagagaucagucucgauuagaucauguucgcaguuucacccguugacu

SEQ ID NO: 491 - RNA, 111 nt, Arabidopsis thaliana
agggaacaagugaagcugccagcaugaucuaucuuugguuaagagaugaauguggaaacauauugcuuaaacccaagcuaggucaugcucugacagccucacuccuuccug

SEQ ID NO: 492 - RNA, 160 nt, Arabidopsis thaliana
ccaguagcaguuaagcugccagcaugaucuugucuuccucucuuagguuucauauauaguuaauaaauauuuuauauauuucuuguucuuacaagauuauaugaucauagcuuagagagagagagagacuaggucaugcugguaguuucaccugcuaaug

SEQ ID NO: 493 - RNA, 361 nt, Arabidopsis thaliana
uuuuagaagcugaagcugccagcaugaucugguaaucgcuacauacgacauacacacaucacuaaacuucuuuauaauuuaugcacacacauacagcucuuaauggccacaacucaaaguuauaauuagugcaugaucucuaguuauuugacugcuuuuaauauauguuuauggauucacgcauguguguguauguacauaauuuacaugcaugcacuuuguguaugguacacaucaauuugaacccguucaaaauucuguuuuuauuaguauauauauagauguauguggugugugugucaguguguguguguguuuauagauaguaguacuaggucauccugcagcuucagucacuaaa

SEQ ID NO: 494 - RNA, 124 nt, Arabidopsis thaliana
gggcucggauucgcuuggugcaggucgggaaccaauucggcugacacagccucgugacuuuuaaaccuuuauugguuugugagcagggauuggaucccgccuugcaucaacugaaucggauccu

SEQ ID NO: 495 - RNA, 109 nt, Arabidopsis thaliana
ggucucggauucgcuuggugcaggucgggaacugauuggcugacaccgacacgugucuugucaugguugguuugugagcucccgucuuguaucaacugaaucggagucc

SEQ ID NO: 496 - RNA, 211 nt, Arabidopsis thaliana
aaguagugugcagccaaggaugacuugccgauuuaaaugaucuuucuuuauacucuauuaagacaauuuaguuucaaacuuuuuuuuuuuuuuuuuuuugaaggauucaggaagaaauuaggauauauuauuccguauaaaauacaagauauauaaaaccaaaaagaaaaaguaacaugaucggcaaguuguccuuggcuacacguuacuu

SEQ ID NO: 497 - RNA, 121 nt, Arabidopsis thaliana
gaguauaaugcagccaaggaugacuugccggaacguuguuaaccaugcauaugaauaaugugaugauuaauuaugugaugaacauauuucuggcaaguuguccuucggcuacauuuugcuc

SEQ ID NO: 498 - RNA, 234 nt, Arabidopsis thaliana
cauuguuguucagccaaggaugacuugccgguagcuuguauuaugauuacucuauauucgauuuauauuauggagaugaugguuuauauauauuuacuuaucuacauaguuuuaguugauuuuuuuucguacguaauauaauacgaaaaaguauuuacuuauuuauauauguguguuggggcaagaaguguaaccaagcuagcccggcaagucaucuauggcuaugcaacuguc

SEQ ID NO: 499 - RNA, 93 nt, Arabidopsis thaliana
aaaugagauugagccaaggaugacuugccgauuuucucaacgaaucuuacugauuaugguauccggcaaguugacuuuggcucuguuuccuuc

SEQ ID NO: 500 - RNA, 123 nt, Arabidopsis thaliana
gaauggaauugagccaaggaugacuugccgguuuaaacccaaccgguuuaugaccauugauuuggucucauucacaaucuguugauucgugucuggcaaguugaccuuggcucugcuucguuc

SEQ ID NO: 501 - RNA, 94 nt, Arabidopsis thaliana
gaaugagauugagccaaggaugacuugccgauguuaucaacaaaucuuaacugauuuugguguccggcaaguugaccuuggcucuguuuccuuc

SEQ ID NO: 502 - RNA, 101 nt, Arabidopsis thaliana
gaaugagguugagccaaggaugacuugccggguuuuuuuaccaaugaaucuaauuaacugauucugguguccggcaaguugaccuuggcucuguuuccuuc

SEQ ID NO: 503 - RNA, 121 nt, Arabidopsis thaliana
acuugugugguagccaaggaugacuugccugcguuuuagaccauauauaucaaagacucacucgaucgauagucuuagaguugguuggucgucaggcagucuccuuggcuauucaaacaau

SEQ ID NO: 504 - RNA, 146 nt, Arabidopsis thaliana
ucauauuugguagccaaggaugacuugccugacucuuuguguaaaauguuuagugucuuguuugaagucacuauaaguuguaucaagcaaugaccauuuugcuuauaaaaaagauaucaggcagucuccuuggcuauccuuauaug

SEQ ID NO: 505 - RNA, 166 nt, Arabidopsis thaliana
ucauguuuaguagccaaggaugacuugccugaucuuuuucaccuccaugauucaauuuguaauucauggguuuuggauuauuauacauucaaaaguauaauaauuugaaaucauguugaaucuugcggguuagguuucaggcagucuccuuggcuaucuugacaug

SEQ ID NO: 506 - RNA, 146 nt, Arabidopsis thaliana
caauauuugguagccaaggaugacuugccugcuucucugaacaaaauggucgaugucauguuuugaagugacuauaaguuauaccaagaaaugaccauuuuguuuauaaauagacaucaggcagucuccuuggcuauccuuauaug

SEQ ID NO: 507 - RNA, 167 nt, Arabidopsis thaliana
ucauguuuaauagccaaggaugacuugccugaucuuuuucaccuccaugauucaauuuuaaguucguggauuuuggauuauuaugcguuuaaaagguauaauaauuugagaucauguugaaucuugcggguuagguuucaggcagucucuuuggcuaucuugacaug

SEQ ID NO: 508 - RNA, 148 nt, Arabidopsis thaliana
ucauauuugguagccaaggaugacuugccuguuucuuugaguaaaauggguuagugucauguuugacaagugacuauaaguuauaucaagcaaugaccauuuuacucaucaaaagacaucaggcagucuccuuggcuauccuuauaug

SEQ ID NO: 509 - RNA, 167 nt, Arabidopsis thaliana
ucauguuuaguagccaaggaugacuugccugaucuuuuucgccuccacgauucaauuucaaauucaugcauuuuggauuauuauaccuuuuaaaguauaauaggucaaauaucauguugaaucuugcggguuagguuucaggcagucucuuuggcuaucuugacaug

SEQ ID NO: 510 - RNA, 84 nt, Arabidopsis thaliana
gagucccucugauauuggccugguucacucagauucucuuuuacuaacucaucugauugagccgugucaauaucucaguccucu

SEQ ID NO: 511 - RNA, 112 nt, Arabidopsis thaliana
gagucccuuugauauuggccugguucacucagaucuuaccugaccacacacguagauauacauuauucucucuagauuaucugauugagccgcgccaauaucucaguacucu

SEQ ID NO: 512 - RNA, 100 nt, Arabidopsis thaliana
gguaacgcgagauauuagugcgguucaaucaaauagucguccucuuaacucauggagaacgguguuguucgauugagccgugccaauaucacgcgguaaa

SEQ ID NO: 513 - RNA, 99 nt, Arabidopsis thaliana
ucaaauacgagauauuggugcgguucaaucagaaaaccguacucuuuuguuuuaaagaucgguuuauuugauugagccgugccaauaucacgcguuuaa

SEQ ID NO: 514 - RNA, 116 nt, Arabidopsis thaliana
uuguuggcugcuguggcaucaucaagauucacaucuguugauggacgguggugauucacucuccacaaaguucucuaugaaaaugagaaucuugaugaugcugcaucggcaaucaa

SEQ ID NO: 515 - RNA, 109 nt, Arabidopsis thaliana
uuguuuguaggcgcagcaccauuaagauucacauggaaauugauaaauacccuaaauuaggguuuugauauguauaugagaaucuugaugaugcugcaucaacaaucga

SEQ ID NO: 516 - RNA, 123 nt, Arabidopsis thaliana
cuguucgcuguuggagcaucaucaagauucacaaaucaucaaguauucguguaaauaaacccauuuaugauuagauuuuugauguauguaugagaaucuugaugaugcugcagcugcaaucag

SEQ ID NO: 517 - RNA, 113 nt, Arabidopsis thaliana
uuguuugcuauugcaacaucuucaagauucagaaaucagauucucuuauggguuuucuuuugagccuuuauuuuuugguuugagaaucuugaugaugcugcagcggcaauuaa

SEQ ID NO: 518 - RNA, 125 nt, Arabidopsis thaliana
guagucgcagaugcagcaccauuaagauucacaagagaugugguucccuuugcuuucgccucucgauccgcagaaaaggguuccuuaucgagugggaaucuugaugaugcugcaucagcaaauac

SEQ ID NO: 519 - RNA, 106 nt, Arabidopsis thaliana
auuaaguacuuucgcuugcagagagaaaucacaguggucaaaaaaguuguaguuuucuuaaagucucuuuccucugugauucucuguguaagcgaaagagcuugcu

SEQ ID NO: 520 - RNA, 133 nt, Arabidopsis thaliana
agaggaaggauccaaagggaucgcauugauccuaauuaaggugaauucuccccauauuuucuuuauaauuggcaaauaaaucacaaaaauuugcuugguuuuggaucaugcuaucucuuuggauucauccuuc

SEQ ID NO: 521 - RNA, 160 nt, Arabidopsis thaliana
agagaaaggauccaaagggaucgcauugauccuaauuaagcugauuuauuccccaauaauuguuuuuuuuuuccuucucaaucgaaagauggaagaaaaacaaauuccaaacauuuugcuuacuuuuccggaucaugcgaucucuuuggauucauucuuu

SEQ ID NO: 522 - RNA, 117 nt, Arabidopsis thaliana
cuuacagucaucuuuggcauucuguccaccuccuucuauacauauaugcauguguauauauauaugcguuucgugugaaagaaggagguggguauacugccaauagagaucuguuag

SEQ ID NO: 523 - RNA, 121 nt, Arabidopsis thaliana
cuuacagagaucuuuggcauucuguccaccuccucucucuauauuuauguguaauaaguguacguaucuacgguguguuucguaagaggaggugggcauacugccaauagagaucuguuag

SEQ ID NO: 524 - RNA, 93 nt, Arabidopsis thaliana
augucuccuagaguuccucugagcacuucauuggggauacaauuuuucuaaaugauuauccacugaaguguuugggggaacucccggacccau

SEQ ID NO: 525 - RNA, 100 nt, Arabidopsis thaliana
auguccccaugaguucccuuuaacgcuucauuguuaaauacucaaagccacauugguuuguauacaacacugaaguguuuggggggacucuuggugucau

SEQ ID NO: 526 - RNA, 100 nt, Arabidopsis thaliana
auguccacaugaguucccuuuaacgcuucauuguugaauacucaaagccacauugguuuguauauaacacugaaguguuuggggggacucuuggugucau

SEQ ID NO: 527 - RNA, 100 nt, Arabidopsis thaliana
auguccucuagaguucuccugaacacuucauuggaaauuuguuauucaguaagcuaacaguuaauuccacugaaguguuugggggaacucccgaugucau

SEQ ID NO: 528 - RNA, 95 nt, Arabidopsis thaliana
auguuuucuagaguuccucugagcacuucauuggagauacaauuuuuuauaaaauaguuuucuacugaaguguuugggggaacucccgggcugau

SEQ ID NO: 529 - RNA, 112 nt, Arabidopsis thaliana
auguccccuugaguucccuuaaacgcuucauuguucauacuuuguuaucaucuaucgaucgaucaaucaaucugaugaacacugaaguguuuggggggacucuaggugacau

SEQ ID NO: 530 - RNA, 151 nt, Arabidopsis thaliana
cucuguauucuuccacagcuuucuugaacugcaaaacuucuucagauuuuuuuuuuuuucuuuugauaucucuuacgcauaaaauagugauuuucuucauaucucugcucgauugauuugcgguucaauaaagcugugggaagauacagac

SEQ ID NO: 531 - RNA, 135 nt, Arabidopsis thaliana
ggucauacuuuuccacagcuuucuugaacuuucuuuuucauuuccauuguuuuuuucuuaaacaaaaguaagaagaaaaaaaacuuuaagauuaagcauuuuggaagcucaagaaagcugugggaaaacaugaca

SEQ ID NO: 532 - RNA, 107 nt, Arabidopsis thaliana
ugaaugaacaucauugagugcagcguugauguaauuucguuuuguuuuucauuguugaauggauuaaaagaauuuauaccagcguugcgcucaauuauguuuuucua

SEQ ID NO: 533 - RNA, 109 nt, Arabidopsis thaliana
ugaaugaacaucauugagugcaucguugauguaauuuuacuuauuuuauuccauuguugaauuaauuaaagaaguauauaucagcguugcauucaauuauguuuuucua

SEQ ID NO: 534 - RNA, 105 nt, Arabidopsis thaliana
ugaaauuucaaaggaguggcaugugaacacauauccuaugguuucuucaaauuuccauugaaaccauugaguuuuguguucucaggucaccccuuugaaucuccc

SEQ ID NO: 535 - RNA, 116 nt, Arabidopsis thaliana
uggaucucgacaggguugauaugagaacacacgaguaaucaacggcuguaaugacgcuacgucauuguuacagcucucguuuucauguguucucaggucaccccugcugagcucuu

SEQ ID NO: 536 - RNA, 115 nt, Arabidopsis thaliana
uggaucucgacaggguugauaugagaacacacgagcaaucaacggcuauaacgacgcuacgucauuguuacagcucucguuucauguguucucaggucaccccugcugagcucuu

SEQ ID NO: 537 - RNA, 123 nt, Arabidopsis thaliana
aaaugcauuacaggguaagaucucuauuggcaggaaaccauuacuuagaucuuugcaucucuuuaugcauugcuuuuaauuagugaguuaucugccaaaggagauuugcccuguaauucuucu

SEQ ID NO: 538 - RNA, 135 nt, Arabidopsis thaliana
ucacuaguuuuagggcgccucuccauuggcagguccuuuacuuccaaauauacacauacauauaugaauaucgaaaauuuccgaugaucgauuuauaaaugaccugccaaaggagaguugcccugaaacugguuc

SEQ ID NO: 539 - RNA, 114 nt, Arabidopsis thaliana
ggagcaguaauagggcaucuuucuauuggcaggcgacuuggcuauuuguaucuuuuguguucuugacuauuggcuaugucacuugccaaaggagaguugcccugucacugcuuc

SEQ ID NO: 540 - RNA, 100 nt, Arabidopsis thaliana
gguuggauuacugggcgaauacuccuauggcagaucgcauuggcuagauaugcaaguaaaaugcuucucugccaaaggagauuugccccgcaauucaucc

SEQ ID NO: 541 - RNA, 109 nt, Arabidopsis thaliana
gaaagcauuacagggcgaauccucuauuggcaguggaaguugaugacccuuauauguuauuuucucaucauuuuccucugccaaaggagauuugccucgcaaugcuuca

SEQ ID NO: 542 - RNA, 118 nt, Arabidopsis thaliana
auaugcauuacagggcaagaucaccauuggcagagaucuauuacuucauucuugcaucauaugcauaaauguuuguggugagcucucugccaaaggagauuugcccgguaauucucuu

SEQ ID NO: 543 - RNA, 21 nt, Oryza sativa
ugacagaagagagugagcaca

SEQ ID NO: 544 - RNA, 21 nt, Oryza sativa
ugacagaagagagugagcaca

SEQ ID NO: 545 - RNA, 21 nt, Oryza sativa
ugacagaagagagugagcaca

SEQ ID NO: 546 - RNA, 21 nt, Oryza sativa
ugacagaagagagugagcaca

SEQ ID NO: 547 - RNA, 21 nt, Oryza sativa
ugacagaagagagugagcaca

SEQ ID NO: 548 - RNA, 21 nt, Oryza sativa
ugacagaagagagugagcaca

SEQ ID NO: 549 - RNA, 21 nt, Oryza sativa
ugacagaagagagugagcaca

SEQ ID NO: 550 - RNA, 21 nt, Oryza sativa
ugacagaagagagugagcaca

SEQ ID NO: 551 - RNA, 21 nt, Oryza sativa
ugacagaagagagugagcaca

SEQ ID NO: 552 - RNA, 21 nt, Oryza sativa
ugacagaagagagugagcaca

SEQ ID NO: 553 - RNA, 21 nt, Oryza sativa
ugacagaagagagagagcaca

SEQ ID NO: 554 - RNA, 21 nt, Oryza sativa
cgacagaagagagugagcaua

SEQ ID NO: 555 - RNA, 21 nt, Oryza sativa
uuuggauugaagggagcucug

SEQ ID NO: 556 - RNA, 21 nt, Oryza sativa
uuuggauugaagggagcucug

SEQ ID NO: 557 - RNA, 21 nt, Oryza sativa
auuggauugaagggagcucca

SEQ ID NO: 558 - RNA, 21 nt, Oryza sativa
auuggauugaagggagcuccg

SEQ ID NO: 559 - RNA, 21 nt, Oryza sativa
auuggauugaagggagcuccu

SEQ ID NO: 560 - RNA, 21 nt, Oryza sativa
cuuggauugaagggagcucua

SEQ ID NO: 561 - RNA, 20 nt, Oryza sativa
uuggacugaagggugcuccc

SEQ ID NO: 562 - RNA, 20 nt, Oryza sativa
uuggacugaagggugcuccc

SEQ ID NO: 563 - RNA, 21 nt, Oryza sativa
ugccuggcucccuguaugcca

SEQ ID NO: 564 - RNA, 21 nt, Oryza sativa
ugccuggcucccuguaugcca

SEQ ID NO: 565 - RNA, 21 nt, Oryza sativa
ugccuggcucccuguaugcca

SEQ ID NO: 566 - RNA, 21 nt, Oryza sativa
ugccuggcucccuguaugcca

SEQ ID NO: 567 - RNA, 21 nt, Oryza sativa
ugccuggcucccuguaugccg

SEQ ID NO: 568 - RNA, 21 nt, Oryza sativa
ugccuggcucccugaaugcca

SEQ ID NO: 569 - RNA, 21 nt, Oryza sativa
ucgauaaaccucugcauccag

SEQ ID NO: 570 - RNA, 21 nt, Oryza sativa
ucgauaagccucugcauccag

SEQ ID NO: 571 - RNA, 21 nt, Oryza sativa
uggagaagcagggcacgugca

SEQ ID NO: 572 - RNA, 21 nt, Oryza sativa
uggagaagcagggcacgugca

SEQ ID NO: 573 - RNA, 21 nt, Oryza sativa
uggagaagcaggguacgugca

SEQ ID NO: 574 - RNA, 21 nt, Oryza sativa
uggagaagcagggcacgugcu

SEQ ID NO: 575 - RNA, 21 nt, Oryza sativa
uggagaagcagggcacgugag

SEQ ID NO: 576 - RNA, 21 nt, Oryza sativa
ucggaccaggcuucauucccc

SEQ ID NO: 577 - RNA, 21 nt, Oryza sativa
ucggaccaggcuucauucccc

SEQ ID NO: 578 - RNA, 21 nt, Oryza sativa
ucggaccaggcuucauucccc

SEQ ID NO: 579 - RNA, 21 nt, Oryza sativa
ucggaccaggcuucauucccc

SEQ ID NO: 580 - RNA, 21 nt, Oryza sativa
ucggaccaggcuucauucccc

SEQ ID NO: 581 - RNA, 21 nt, Oryza sativa
ucggaccaggcuucauucccc

SEQ ID NO: 582 - RNA, 21 nt, Oryza sativa
ucggaccaggcuucauuccuc

SEQ ID NO: 583 - RNA, 21 nt, Oryza sativa
ucggaccaggcuucauuccuc

SEQ ID NO: 584 - RNA, 21 nt, Oryza sativa
ucggaucaggcuucauuccuc

SEQ ID NO: 585 - RNA, 21 nt, Oryza sativa
ucggaucaggcuucauuccuc

SEQ ID NO: 586 - RNA, 21 nt, Oryza sativa
ucggaccaggcuucaaucccu

SEQ ID NO: 587 - RNA, 21 nt, Oryza sativa
ucggaccaggcuucaaucccu

SEQ ID NO: 588 - RNA, 21 nt, Oryza sativa
ugaagcugccagcaugaucua

SEQ ID NO: 589 - RNA, 21 nt, Oryza sativa
ugaagcugccagcaugaucua

SEQ ID NO: 590 - RNA, 21 nt, Oryza sativa
ugaagcugccagcaugaucua

SEQ ID NO: 591 - RNA, 21 nt, Oryza sativa
ugaagcugccagcaugaucug

SEQ ID NO: 592 - RNA, 21 nt, Oryza sativa
ugaagcugccagcaugaucug

SEQ ID NO: 593 - RNA, 21 nt, Oryza sativa
ugaagcugccagcaugaucug

SEQ ID NO: 594 - RNA, 21 nt, Oryza sativa
ugaagcugccagcaugaucug

SEQ ID NO: 595 - RNA, 21 nt, Oryza sativa
ugaagcugccagcaugaucug

SEQ ID NO: 596 - RNA, 21 nt, Oryza sativa
ugaagcugccagcaugaucug

SEQ ID NO: 597 - RNA, 21 nt, Oryza sativa
ucgcuuggugcagaucgggac

SEQ ID NO: 598 - RNA, 21 nt, Oryza sativa
aggcuuggugcagcucgggaa

SEQ ID NO: 599 - RNA, 21 nt, Oryza sativa
cagccaaggaugacuugccga

SEQ ID NO: 600 - RNA, 21 nt, Oryza sativa
cagccaaggaugacuugccgg

SEQ ID NO: 601 - RNA, 21 nt, Oryza sativa
cagccaaggaugacuugccgg

SEQ ID NO: 602 - RNA, 21 nt, Oryza sativa
uagccaaggaugaauugccgg

SEQ ID NO: 603 - RNA, 21 nt, Oryza sativa
uagccaaggaugacuugccgg

SEQ ID NO: 604 - RNA, 21 nt, Oryza sativa
uagccaaggaugacuugccua

SEQ ID NO: 605 - RNA, 21 nt, Oryza sativa
uagccaaggaugacuugccua

SEQ ID NO: 606 - RNA, 21 nt, Oryza sativa
uagccaaggaugacuugccug

SEQ ID NO: 607 - RNA, 21 nt, Oryza sativa
uagccaaggaugacuugccug

SEQ ID NO: 608 - RNA, 21 nt, Oryza sativa
uagccaaggaugacuugccug

SEQ ID NO: 609 - RNA, 21 nt, Oryza sativa
uagccaaggaugacuugccug

SEQ ID NO: 610 - RNA, 21 nt, Oryza sativa
uagccaaggaugacuugccug

SEQ ID NO: 611 - RNA, 21 nt, Oryza sativa
uagccaaggaugacuugccug

SEQ ID NO: 612 - RNA, 21 nt, Oryza sativa
uagccaagaaugacuugccua

SEQ ID NO: 613 - RNA, 21 nt, Oryza sativa
uagccaagaaugacuugccua

SEQ ID NO: 614 - RNA, 22 nt, Oryza sativa
uagccaaggacaaacuugccgg

SEQ ID NO: 615 - RNA, 21 nt, Oryza sativa
uagccaaggagacugcccaug

SEQ ID NO: 616 - RNA, 21 nt, Oryza sativa
ugauugagccgcgccaauauc

SEQ ID NO: 617 - RNA, 21 nt, Oryza sativa
ugauugagccgugccaauauc

SEQ ID NO: 618 - RNA, 21 nt, Oryza sativa
ugauugagccgugccaauauc

SEQ ID NO: 619 - RNA, 21 nt, Oryza sativa
ugauugagccgugccaauauc

SEQ ID NO: 620 - RNA, 21 nt, Oryza sativa
ugauugagccgugccaauauc

SEQ ID NO: 621 - RNA, 21 nt, Oryza sativa
ugauugagccgugccaauauc

SEQ ID NO: 622 - RNA, 21 nt, Oryza sativa
gaggugagccgagccaauauc

SEQ ID NO: 623 - RNA, 21 nt, Oryza sativa
agaaucuugaugaugcugcau

SEQ ID NO: 624 - RNA, 21 nt, Oryza sativa
ggaaucuugaugaugcugcau

SEQ ID NO: 625 - RNA, 21 nt, Oryza sativa
ugaaucuugaugaugcugcac

SEQ ID NO: 626 - RNA, 21 nt, Oryza sativa
uccaaagggaucgcauugauc

SEQ ID NO: 627 - RNA, 20 nt, Oryza sativa
uuggcauucuguccaccucc

SEQ ID NO: 628 - RNA, 21 nt, Oryza sativa
gugaagugcuugggggaacuc

SEQ ID NO: 629 - RNA, 21 nt, Oryza sativa
gugaaguguuugggggaacuc

SEQ ID NO: 630 - RNA, 21 nt, Oryza sativa
gugaaguguuuggaggaacuc

SEQ ID NO: 631 - RNA, 21 nt, Oryza sativa
gugaaguguuugggggaacuc

SEQ ID NO: 632 - RNA, 21 nt, Oryza sativa
gugaaguguuugggggaacuc

SEQ ID NO: 633 - RNA, 21 nt, Oryza sativa
gugaaguauuugggggaacuc

SEQ ID NO: 634 - RNA, 21 nt, Oryza sativa
gugaaguguuugggggaacuc

SEQ ID NO: 635 - RNA, 21 nt, Oryza sativa
gugaaguguuugggggaacuc

SEQ ID NO: 636 - RNA, 21 nt, Oryza sativa
gugaaguguuugggggaacuc

SEQ ID NO: 637 - RNA, 21 nt, Oryza sativa
gugaaguguuugggggaacuc

SEQ ID NO: 638 - RNA, 21 nt, Oryza sativa
gugaaguguuugggggaacuc

SEQ ID NO: 639 - RNA, 21 nt, Oryza sativa
gugaaguguuugggggaacuc

SEQ ID NO: 640 - RNA, 21 nt, Oryza sativa
gugaaguguuugggggaacuc

SEQ ID NO: 641 - RNA, 21 nt, Oryza sativa
gugaaguguuugggggaacuc

SEQ ID NO: 642 - RNA, 21 nt, Oryza sativa
gugaaguguuuggggaaacuc

SEQ ID NO: 643 - RNA, 21 nt, Oryza sativa
gugaagcguuugggggaaauc

SEQ ID NO: 644 - RNA, 21 nt, Oryza sativa
gugaaguguuuggggaaacuc

SEQ ID NO: 645 - RNA, 21 nt, Oryza sativa
gugaaguguuuggggaaacuc

SEQ ID NO: 646 - RNA, 21 nt, Oryza sativa
gugaagcguuugggggaaauc

SEQ ID NO: 647 - RNA, 21 nt, Oryza sativa
uuccacagcuuucuugaacug

SEQ ID NO: 648 - RNA, 21 nt, Oryza sativa
uuccacagcuuucuugaacug

SEQ ID NO: 649 - RNA, 21 nt, Oryza sativa
uuccacagcuuucuugaacuu

SEQ ID NO: 650 - RNA, 21 nt, Oryza sativa
ucauugagugcagcguugaug

SEQ ID NO: 651 - RNA, 21 nt, Oryza sativa
uuauugagugcagcguugaug

SEQ ID NO: 652 - RNA, 21 nt, Oryza sativa
uguguucucaggucaccccuu

SEQ ID NO: 653 - RNA, 21 nt, Oryza sativa
uguguucucaggucgccccug

SEQ ID NO: 654 - RNA, 21 nt, Oryza sativa
ugccaaaggagaauugcccug

SEQ ID NO: 655 - RNA, 21 nt, Oryza sativa
ugccaaaggagaauugcccug

SEQ ID NO: 656 - RNA, 21 nt, Oryza sativa
ugccaaaggagaauugcccug

SEQ ID NO: 657 - RNA, 21 nt, Oryza sativa
ugccaaaggagaguugcccug

SEQ ID NO: 658 - RNA, 21 nt, Oryza sativa
ugccaaaggagauuugcccag

SEQ ID NO: 659 - RNA, 21 nt, Oryza sativa
ugccaaaggagauuugcccag

SEQ ID NO: 660 - RNA, 21 nt, Oryza sativa
ugccaaaggagauuugcccgg

SEQ ID NO: 661 - RNA, 21 nt, Oryza sativa
ugccaaaggagacuugcccag

SEQ ID NO: 662 - RNA, 21 nt, Oryza sativa
ugccaaaggagagcugcccug

SEQ ID NO: 663 - RNA, 21 nt, Oryza sativa
ugccaaaggagaguugcccua

SEQ ID NO: 664 - RNA, 21 nt, Oryza sativa
ugccaaaggaaauuugccccg

SEQ ID NO: 665 - RNA, 31 nt, Oryza sativa
ggugacagaagagagugagcacacgugguug

SEQ ID NO: 666 - RNA, 32 nt, Oryza sativa
gucugacagaagagagugagcacacacggugc

SEQ ID NO: 667 - RNA, 32 nt, Oryza sativa
ggcugacagaagagagugagcacacaugguga

SEQ ID NO: 668 - RNA, 29 nt, Oryza sativa
uugacagaagagagugagcacacagcgug

SEQ ID NO: 669 - RNA, 26 nt, Oryza sativa
ggugacagaagagagugagcacacgg

SEQ ID NO: 670 - RNA, 25 nt, Oryza sativa
aguugacagaagagagugagcacac

SEQ ID NO: 671 - RNA, 31 nt, Oryza sativa
ggcugacagaagagagugagcacacagcggg

SEQ ID NO: 672 - RNA, 28 nt, Oryza sativa
uuguugacagaagagagugagcacacgg

SEQ ID NO: 673 - RNA, 26 nt, Oryza sativa
ggugacagaagagagugagcacacgg

SEQ ID NO: 674 - RNA, 28 nt, Oryza sativa
uuguugacagaagagagugagcacacgg

SEQ ID NO: 675 - RNA, 20 nt, Oryza sativa
ugacagaagagagagagcac

SEQ ID NO: 676 - RNA, 26 nt, Oryza sativa
gccgacagaagagagugagcauauau

SEQ ID NO: 677 - RNA, 23 nt, Oryza sativa
ucuuuggauugaagggagcucug

SEQ ID NO: 678 - RNA, 25 nt, Oryza sativa
acucuuuggauugaagggagcucug

SEQ ID NO: 679 - RNA, 24 nt, Oryza sativa
ugauuggauugaagggagcuccac

SEQ ID NO: 680 - RNA, 22 nt, Oryza sativa
ugauuggauugaagggagcucc

SEQ ID NO: 681 - RNA, 24 nt, Oryza sativa
uugauuggauugaagggagcuccu

SEQ ID NO: 682 - RNA, 26 nt, Oryza sativa
uuaugcuuggauugaagggagcucua

SEQ ID NO: 683 - RNA, 21 nt, Oryza sativa
ugguuggacugaagggugcuc

SEQ ID NO: 684 - RNA, 28 nt, Oryza sativa
gugugccuggcucccuguaugccacaca

SEQ ID NO: 685 - RNA, 28 nt, Oryza sativa
agcgugccuggcucccuguaugccacuc

SEQ ID NO: 686 - RNA, 27 nt, Oryza sativa
augugccuggcucccuguaugccacuc

SEQ ID NO: 687 - RNA, 29 nt, Oryza sativa
gauaugccuggcucccuguaugccacucg

SEQ ID NO: 688 - RNA, 27 nt, Oryza sativa
ggauaugccuggcucccuguaugccgc

SEQ ID NO: 689 - RNA, 24 nt, Oryza sativa
cugccuggcucccugaaugccauc

SEQ ID NO: 690 - RNA, 30 nt, Oryza sativa
ggaaucgaucgauaaaccucugcauccagu

SEQ ID NO: 691 - RNA, 29 nt, Oryza sativa
gaaucgaucgauaagccucugcauccaga

SEQ ID NO: 692 - RNA, 25 nt, Oryza sativa
acgguggagaagcagggcacgugca

SEQ ID NO: 693 - RNA, 30 nt, Oryza sativa
ccgcguuggagaagcagggcacgugcaugc

SEQ ID NO: 694 - RNA, 26 nt, Oryza sativa
uuguuggagaagcaggguacgugcaa

SEQ ID NO: 695 - RNA, 28 nt, Oryza sativa
ccgugcuggagaagcagggcacgugcuc

SEQ ID NO: 696 - RNA, 26 nt, Oryza sativa
aggguggagaagcagggcacgugagc

SEQ ID NO: 697 - RNA, 28 nt, Oryza sativa
ucucggaccaggcuucauuccccucaga

SEQ ID NO: 698 - RNA, 25 nt, Oryza sativa
ucucggaccaggcuucauucccccc

SEQ ID NO: 699 - RNA, 24 nt, Oryza sativa
uucggaccaggcuucauucccccc

SEQ ID NO: 700 - RNA, 29 nt, Oryza sativa
ucucggaccaggcuucauuccccucaagu

SEQ ID NO: 701 - RNA, 28 nt, Oryza sativa
ucucggaccaggcuucauuccccucaga

SEQ ID NO: 702 - RNA, 29 nt, Oryza sativa
ucucggaccaggcuucauuccccucaaca

SEQ ID NO: 703 - RNA, 26 nt, Oryza sativa
ucucggaccaggcuucauuccucaca

SEQ ID NO: 704 - RNA, 25 nt, Oryza sativa
ucggaccaggcuucauuccucgcaa

SEQ ID NO: 705 - RNA, 28 nt, Oryza sativa
ggagccucggaccaggcuucaaucccuu

SEQ ID NO: 706 - RNA, 23 nt, Oryza sativa
cucggaccaggcuucaaucccuu

SEQ ID NO: 707 - RNA, 30 nt, Oryza sativa
gagugaagcugccagcaugaucuagcucug

SEQ ID NO: 708 - RNA, 27 nt, Oryza sativa
cgugaagcugccagcaugaucuaacuu

SEQ ID NO: 709 - RNA, 28 nt, Oryza sativa
gagugaagcugccagcaugaucuagcuc

SEQ ID NO: 710 - RNA, 28 nt, Oryza sativa
agcugaagcugccagcaugaucugauga

SEQ ID NO: 711 - RNA, 24 nt, Oryza sativa
augaagcugccagcaugaucuggu

SEQ ID NO: 712 - RNA, 29 nt, Oryza sativa
uggaugaagcugccagcaugaucugauca

SEQ ID NO: 713 - RNA, 22 nt, Oryza sativa
ggugaagcugccagcaugaucu

SEQ ID NO: 714 - RNA, 29 nt, Oryza sativa
uuggugaagcugccagcaugaucugauga

SEQ ID NO: 715 - RNA, 26 nt, Oryza sativa
ggcugaagcugccagcaugaucuggu

SEQ ID NO: 716 - RNA, 30 nt, Oryza sativa
cucgggcucgcuuggugcagaucgggaccc

SEQ ID NO: 717 - RNA, 29 nt, Oryza sativa
auggugcagccaaggaugacuugccgauc

SEQ ID NO: 718 - RNA, 26 nt, Oryza sativa
aaugcagccaaggaugacuugccggu

SEQ ID NO: 719 - RNA, 29 nt, Oryza sativa
ggaugcagccaaggaugacuugccggcuc

SEQ ID NO: 720 - RNA, 25 nt, Oryza sativa
guguagccaaggaugaauugccggc

SEQ ID NO: 721 - RNA, 25 nt, Oryza sativa
uucgguagccaaggaugacuugccu

SEQ ID NO: 722 - RNA, 22 nt, Oryza sativa
cucugguagccaaggaugacuu

SEQ ID NO: 723 - RNA, 22 nt, Oryza sativa
cucugguagccaaggaugacuu

SEQ ID NO: 724 - RNA, 22 nt, Oryza sativa
cucugguagccaaggaugacuu

SEQ ID NO: 725 - RNA, 22 nt, Oryza sativa
aucugguagccaaggaugacuu

SEQ ID NO: 726 - RNA, 20 nt, Oryza sativa
uagauagccaaggaugacuu

SEQ ID NO: 727 - RNA, 21 nt, Oryza sativa
ccugguagccaaggaugacuu

SEQ ID NO: 728 - RNA, 21 nt, Oryza sativa
uuugguagccaagaaugacuu

SEQ ID NO: 729 - RNA, 21 nt, Oryza sativa
uuugguagccaagaaugacuu

SEQ ID NO: 730 - RNA, 21 nt, Oryza sativa
agcaagguguagccaaggaca

SEQ ID NO: 731 - RNA, 21 nt, Oryza sativa
ucaggcuagccaaggagacug

SEQ ID NO: 732 - RNA, 28 nt, Oryza sativa
guaucugauugagccgcgccaauaucuc

SEQ ID NO: 733 - RNA, 31 nt, Oryza sativa
ucuuuugauugagccgugccaauaucacguc

SEQ ID NO: 734 - RNA, 31 nt, Oryza sativa
cucuuugauugagccgugccaauaucacguc

SEQ ID NO: 735 - RNA, 29 nt, Oryza sativa
uucugauugagccgugccaauaucucagc

SEQ ID NO: 736 - RNA, 29 nt, Oryza sativa
uuucugauugagccgugccaauaucuuag

SEQ ID NO: 737 - RNA, 26 nt, Oryza sativa
gucugauugagccgugccaauaucac

SEQ ID NO: 738 - RNA, 20 nt, Oryza sativa
gugagccgagccaauaucac

SEQ ID NO: 739 - RNA, 31 nt, Oryza sativa
ggcugagaaucuugaugaugcugcauccgca

SEQ ID NO: 740 - RNA, 27 nt, Oryza sativa
gggaaucuugaugaugcugcaucggaa

SEQ ID NO: 741 - RNA, 31 nt, Oryza sativa
ugcgugaaucuugaugaugcugcaccagcaa

SEQ ID NO: 742 - RNA, 34 nt, Oryza sativa
ggggaagcauccaaagggaucgcauugauccuuc

SEQ ID NO: 743 - RNA, 32 nt, Oryza sativa
gagaguucuuuggcauucuguccaccuccuug

SEQ ID NO: 744 - RNA, 25 nt, Oryza sativa
agugaagugcuugggggaacuccag

SEQ ID NO: 745 - RNA, 25 nt, Oryza sativa
cgugaaguguuugggggaacucuua

SEQ ID NO: 746 - RNA, 25 nt, Oryza sativa
gugaaguguuuggaggaacucucgg

SEQ ID NO: 747 - RNA, 27 nt, Oryza sativa
ugugaaguguuugggggaacucucggu

SEQ ID NO: 748 - RNA, 26 nt, Oryza sativa
ugugaaguguuugggggaacucucga

SEQ ID NO: 749 - RNA, 25 nt, Oryza sativa
gugaaguauuugggggaacucucga

SEQ ID NO: 750 - RNA, 25 nt, Oryza sativa
ugugaaguguuugggggaacucucg

SEQ ID NO: 751 - RNA, 33 nt, Oryza sativa
guagugaaguguuugggggaacucuagguggca

SEQ ID NO: 752 - RNA, 27 nt, Oryza sativa
ugugaaguguuugggggaacucuuggu

SEQ ID NO: 753 - RNA, 32 nt, Oryza sativa
cugugaaguguuugggggaacucuagguggca

SEQ ID NO: 754 - RNA, 26 nt, Oryza sativa
ugugaaguguuugggggaacucuuga

SEQ ID NO: 755 - RNA, 26 nt, Oryza sativa
uugugaaguguuugggggaacucuug

SEQ ID NO: 756 - RNA, 20 nt, Oryza sativa
guguuugggggaacucucga

SEQ ID NO: 757 - RNA, 25 nt, Oryza sativa
agugaaguguuuggggaaacuccgg

SEQ ID NO: 758 - RNA, 25 nt, Oryza sativa
agugaaguguuuggggaaacuccgg

SEQ ID NO: 759 - RNA, 25 nt, Oryza sativa
agugaaguguuuggggaaacuccgg

SEQ ID NO: 760 - RNA, 25 nt, Oryza sativa
aucuuccacagcuuucuugaacugc

SEQ ID NO: 761 - RNA, 25 nt, Oryza sativa
gucuuccacagcuuucuugaacugc

SEQ ID NO: 762 - RNA, 30 nt, Oryza sativa
caugccuuuccacagcuuucuugaacuucu

SEQ ID NO: 763 - RNA, 26 nt, Oryza sativa
gcaucauugagugcagcguugaugaa

SEQ ID NO: 764 - RNA, 27 nt, Oryza sativa
acuguguucucaggucaccccuuuggg

SEQ ID NO: 765 - RNA, 26 nt, Oryza sativa
cguguguucucaggucgccccugccg

SEQ ID NO: 766 - RNA, 23 nt, Oryza sativa
gugccaaaggagaauugcccugc

SEQ ID NO: 767 - RNA, 25 nt, Oryza sativa
cgugccaaaggagaauugcccugcc

SEQ ID NO: 768 - RNA, 24 nt, Oryza sativa
cgugccaaaggagaauugcccugc

SEQ ID NO: 769 - RNA, 26 nt, Oryza sativa
accacugccaaaggagauuugcccag

SEQ ID NO: 770 - RNA, 30 nt, Oryza sativa
uguucucucugccaaaggagauuugcccag

SEQ ID NO: 771 - RNA, 27 nt, Oryza sativa
ucugccaaaggagauuugcccggcgau

SEQ ID NO: 772 - RNA, 28 nt, Oryza sativa
ccacugccaaaggagacuugcccagcaa

SEQ ID NO: 773 - RNA, 27 nt, Oryza sativa
cccugccaaaggagagcugcccugcca

SEQ ID NO: 774 - RNA, 22 nt, Oryza sativa
ggagaguugcccuaaaacugga

SEQ ID NO: 775 - RNA, 30 nt, Oryza sativa
acugccaaaggaaauuugccccggaauuca

SEQ ID NO: 776 - RNA, 109 nt, Oryza sativa
acuaggagggugacagaagagagugagcacacgugguuguuuccuugcauaaaugaugccuaugcuuggagcuacgcgugcucacuucucucucugucaccuccacccc

SEQ ID NO: 777 - RNA, 106 nt, Oryza sativa
uuuggaggucugacagaagagagugagcacacacggugcuuucuuagcaugcaagagccaugcugggagcugugcgugcucacucucuaucugucagccguucacc

SEQ ID NO: 778 - RNA, 111 nt, Oryza sativa
gaggugaggcugacagaagagagugagcacacauggugacuuucuugcaugcugaauggacucaugcuugaagcuaugugugcucacuucucucucugucagccauuugau

SEQ ID NO: 779 - RNA, 104 nt, Oryza sativa
cucaugagauugacagaagagagugagcacacagcgugauggccggcauaaaaucuaucccguccucgccgcgugcucacuccucuuucugucacccucuuucu

SEQ ID NO: 780 - RNA, 106 nt, Oryza sativa
uggcgcgaggugacagaagagagugagcacacggccgggcgugacggcaccggcgggcgugccgucgcggccgcgugcucacugcucuuucugucauccggugccg

SEQ ID NO: 781 - RNA, 203 nt, Oryza sativa
ugguggcaguugacagaagagagugagcacacagcggccagacugcaucgaucuaucaaucuucccuucgacaggauagcuagauagaaagaaagagaggccgucggcggccauggaagagagagagagagagagagaugaaaugaugaugaugauacagcugccgcucgcgugcucacuucucuuucugucagcucucccug

SEQ ID NO: 782 - RNA, 132 nt, Oryza sativa
cgcggcuggcugacagaagagagugagcacacagcgggcagacugcaucugaaauaaacuggugacgacgaagaagacgacggacgcagcuugccguugcgugcucacuucucucucugucagcucucucug

SEQ ID NO: 783 - RNA, 106 nt, Oryza sativa
gcgagauuguugacagaagagagugagcacacggcgcggcggcuagccaucggcgggaugccugcccccgccgcgugcucgcuccucuuucugucagcaucucuca

SEQ ID NO: 784 - RNA, 106 nt, Oryza sativa
cgcugggcggugacagaagagagugagcacacggccgggcggaacggcaccggcggaugugccgucgcggccgcgugcucacugcucugucugucauccacuccuc

SEQ ID NO: 785 - RNA, 106 nt, Oryza sativa; identical to SEQ ID NO: 783

SEQ ID NO: 786 - RNA, 106 nt, Oryza sativa
uugagagugaugacagaagagagagagcacaacccgacagcagcgacgacggcggucgcuucugccagggccgugugcucucugaucuaucugucaucgccgucca

SEQ ID NO: 787 - RNA, 156 nt, Oryza sativa
gcuagggagccgacagaagagagugagcauauauaguucuuuccuugcauauguggucauauguguguugacugaagagauacauauauauagagagagagaguucaugugcuugaagcuauaugugcucacuucucuuucugucagcaaauuauc

SEQ ID NO: 788 - RNA, 272 nt, Oryza sativa
guuguggacguugagcuccuuucgguccaaaaagggguguugcugugggucgauugagcugcugggucauggaucccguuagccuacuccauguucaucauucagcucgagaucugaaagaaacuacuccaauuuauacuaauaguauguguguagauaggaaaaugauggaguacucguuguugggauaggcuuauggcuugcaugccccaggagcugcaucaacccuacauggacccucuuuggauugaagggagcucugcaucuuuggu

SEQ ID NO: 789 - RNA, 188 nt, Oryza sativa
gguuaugaaguggagcuccuuucguuccaaugaaagguuuaucugaagggugauacagcugcuuguucaugguucccacuauucuaucucauaggaaaagagauaggcuugugguuugcaugaccaaggagccgaaucaacuccuugcugaccacucuuuggauugaagggagcucugcaucuugauc

SEQ ID NO: 790 - RNA, 197 nt, Oryza sativa
gaggaggaagaggagcuccuuucgauccaauucaggagaggaagugguaggaugcagcugccgauucauggauaccucuggagugcguggcagcaaugcuguaggccugcacuugcauggguuugcaugacccgggagaugaacccaccauugucuuccucuauugauuggauugaagggagcuccacaucucucuc

SEQ ID NO: 791 - RNA, 189 nt, Oryza sativa
ugaugugaggaggagcuccuuucgauccaauucaggagaggaaguggugggaugcagcugccgguucauggauaccucugcaguucaugccgguaggccugcacuugcauggguuugcaugaccugggagaugaaccugccauuguguuccucuauugauuggauugaagggagcuccggcuacaccua

SEQ ID NO: 792 - RNA, 191 nt, Oryza sativa
gaugaagaagaagagcucccuuucgauccaauucaggagaggaagugguaggaugcagcugccgguucauggauaccucuggagugcagggcaaauaguccuacccuuucauggguuugcaugacucgggagaugaacccgccauugucuuccucuauugauuggauugaagggagcuccucuagcuacau

SEQ ID NO: 793 - RNA, 188 nt, Oryza sativa
gaagaagaagacgagcucccuucgauccaauccaggagaggaagugguaggaugcagcugccgguucauggauaccucugcagugcaugucguaggcuugcacuugcauggguuugcaugacccgggagaugaacccaccauugucuuccucuuaugcuuggauugaagggagcucuacaccucucuc

SEQ ID NO: 794 - RNA, 191 nt, Oryza sativa
uguguaagaagagagcucucuucaguccacucucagauggcuguaggguuuuauuagcugccgaaucauccauucaccuaccaagaaaguugcaggaguguaucucuugguagcggacuggaugacgcgggagcuaaaauuuagcucugcgccguuugugguuggacugaagggugcucccuugcucaagc

SEQ ID NO: 795 - RNA, 197 nt, Oryza sativa
gauggauggaagagagcguccuucaguccacucaugggcggugcuagggucgaauuagcugccgacucauucacccacaugccaagcaagaaacgcuugagauagcgaagcuuagcagaugagugaaugaagcgggagguaacguuccgaucucgcgccgucuuugcuuggacugaagggugcucccuccuccucga

SEQ ID NO: 796 - RNA, 102 nt, Oryza sativa
guguagugugugccuggcucccuguaugccacacauguagaccaacccauggugucugguugccuacuggguggcgugcaaggagccaagcaugcaugccug

SEQ ID NO: 797 - RNA, 102 nt, Oryza sativa
cuugagagcgugccuggcucccuguaugccacucauguagcccaauccaugguguguuuggaugcuguggguggcgugcaaggagccaagcaugcgugccau

SEQ ID NO: 798 - RNA, 107 nt, Oryza sativa
auugggaaugugccuggcucccuguaugccacucaucuagagcaacaaacuucugcgagagguugccuaugauggauggcgugcacggagccaagcauauucccucc

SEQ ID NO: 799 - RNA, 150 nt, Oryza sativa
aaaggggauaugccuggcucccuguaugccacucgcguagcugccaaacucaguugaaacaacugccuucucccggcgagauucaggcauuguguucguacguuuggcucuacugcggauggcgugcgaggagccaagcaugaccgucuc

SEQ ID NO: 800 - RNA, 114 nt, Oryza sativa
guaggggauaugccuggcucccuguaugccgcucgcauggcugccaacccaaugaacucgaucucguuguuggccgcugcguacggcgugcgaggugccaagcauggcccucuu

SEQ ID NO: 801 - RNA, 100 nt, Oryza sativa
ggauuaacgcugccuggcucccugaaugccauccgagaagcgugccgcuguggccggcugcuuccugguuggcauugagggagucaugcaggguuugcuc

SEQ ID NO: 802 - RNA, 176 nt, Oryza sativa
guggugaugccugggcgcagugguuuaucgaucccuucccugccuuguggcgcugauccaggagcggcgaauuucuuugagaggguguucuuuuuuuuuucuuccuuuugguccuuguugcagccaacgacaacgcgggaaucgaucgauaaaccucugcauccaguucucgccuu

SEQ ID NO: 803 - RNA, 134 nt, Oryza sativa
ugggugaugccugggcgcagugguuuaucgaucucuucccugccuugugcugcuccgaucgaugcccgugcugauucuugauaauauacaacgcaggaaucgaucgauaagccucugcauccagaucucacuuu

SEQ ID NO: 804 - RNA, 113 nt, Oryza sativa
ccgugcacgguggagaagcagggcacgugcauuaccauccacucgccugccggccgccggccgccauugccauggaugguucuucaugugcccgucuucuccaccgagcacua

SEQ ID NO: 805 - RNA, 142 nt, Oryza sativa
aggaccgcguuggagaagcagggcacgugcaugcauauguucaucaucaucuucuuccuccuccucuagcuccagccuuguguggguuggaaguuuagauagaacucgcacugcacguggucuccuucuccaucccggucuu

SEQ ID NO: 806 - RNA, 119 nt, Oryza sativa
agguucuuguuggagaagcaggguacgugcaaaaugcacaccgguuggucgagcuaauuaacaagcucugacgaccauggugaucgaaugcacgugcuccccuucuccaccauggccuu

SEQ ID NO: 807 - RNA, 94 nt, Oryza sativa
caaaccgugcuggagaagcagggcacgugcucgacggcggggcuggcuggccggccggcuugcagcaugugcgcuccuucuccagcauggcuuc

SEQ ID NO: 808 - RNA, 132 nt, Oryza sativa
uugugcaggguggagaagcagggcacgugagcggccauccaguguagcuucgcugcgcguccauggcggcgaacgcgcgugaucuggaguuuggauggucguucauguguccgucuucuccaccgagcacug

SEQ ID NO: 809 - RNA, 131 nt, Oryza sativa
uugcuucugaguggaauguugucugguucaaggucucauacaccuugugguuuugaggaugauuugugcaagguuuuucauuccucucauccgugggaucucggaccaggcuucauuccccucagagauag

SEQ ID NO: 810 - RNA, 190 nt, Oryza sativa
uucauuuugaggggaauguugucuggcucggggcuacuuuuaauuucucucucuuuugauaucuucuuuucucgaucuccuagcuugaucuuuuugaucucucaaaucgaucuuaagaaaaagaucagucaaagagaugagaguagaugucuguagaucucggaccaggcuucauuccccccaaacagaa

SEQ ID NO: 811 - RNA, 122 nt, Oryza sativa
uggcaguugaggggaauguugucugguccgagaccuaacaccgggcggaauggcggauucagcugcagcuaagcaagcuaggugggggguuucggaccaggcuucauuccccccaacucaac

SEQ ID NO: 812 - RNA, 113 nt, Oryza sativa
uucacuuugaggggaauguugucuggcucgaggugcauggagaaaccucugaucgaucagguuugaucuguagagacugaucucggaccaggcuucauuccccucaaguaaag

SEQ ID NO: 813 - RNA, 139 nt, Oryza sativa
uuguuuuuggguggaauguugucugguucaaggccccuuaggaugugugauuuuugaugguuuaugcauucaucuugaugcgaacaucuaucucggaucuuuggguucucggaccaggcuucauuccccucagagauag

SEQ ID NO: 814 - RNA, 97 nt, Oryza sativa
aucuuguugagaggaauguugucuggccugagaucguaccauagugguggguacacguggacggucucggaccaggcuucauuccccucaacaacuc

SEQ ID NO: 815 - RNA, 145 nt, Oryza sativa
agcauggugucuggaauggaggcugauccaagauccuugcuuggugcaaaauacuagggcauuguuguaagugccauuaguucuuuuuuguuuccgaguuuguuaucgaggaucucggaccaggcuucauuccucacaccgugcu

SEQ ID NO: 816 - RNA, 119 nt, Oryza sativa
gguggcuuguggggaauguuggcuggcucgaggcauccacaucuuaauuccucuccggcgaucgagccggcucgggcguguggaggcgucggaccaggcuucauuccucgcaagccgau

SEQ ID NO: 817 - RNA, 145 nt, Oryza sativa
agauagguguuuggaaugcaguuugauccaagaucugccuauauauaugguguguauaucauaucuugugauaugggggauaugcaacaagugugugacagggguggguagaucucggaucaggcuucauuccucacaccaauac

SEQ ID NO: 818 - RNA, 140 nt, Oryza sativa
agauagguguuuggaaugcaguuugauccaagaucugccuauauauaugguguguauaucauaucuugugauaugggggauaugcaacaagugugugacagggguggguagaucucggaucaggcuucauuccucacacc

SEQ ID NO: 819 - RNA, 127 nt, Oryza sativa
auuagguuaagggguuuguugucuggcucgaggcauccgggacuccgguuucuccuuaccuacuggaggcgccuagcuuccggcgagcucggagccucggaccaggcuucaaucccuuuaaccaugc

SEQ ID NO: 820 - RNA, 117 nt, Oryza sativa
guuagguuaaggggauuguugucugguucaaggucuccacauugugcaaaauguucauucauggaggcacaggaugcuuggugaucucggaccaggcuucaaucccuuuaaccagca

SEQ ID NO: 821 - RNA, 135 nt, Oryza sativa
ugugaaugagugaagcugccagcaugaucuagcucugauuaaucggcacuguuggcguacagucgauugacuaaucgucagaucuguguguguaaaucacuguuagaucaugcaugacagccucauuucuucaca

SEQ ID NO: 822 - RNA, 147 nt, Oryza sativa
agagaaagcgugaagcugccagcaugaucuaacuugcagacaagaaaucagcucagcucgcugguuucgaacaggaaggcggcuagcugaggcuucuucugaguacgugaugguuagaucaugcugugacaguuucacuccuucccu

SEQ ID NO: 823 - RNA, 147 nt, Oryza sativa
agggaacgagugaagcugccagcaugaucuagcucugaaugaucaacaagaugugcucccacacugccuuccuguggaucuugagcuguugcuagucuuguggucaugccuugcuaggucaugcugcggcagccucacuucuuccca

SEQ ID NO: 824 - RNA, 110 nt, Oryza sativa
cauuaggagcugaagcugccagcaugaucugaugagugcuuauuaggugagggcagaauugacugccaaaacaaagaucagaucaugcugugcaguuucaucugcuugug

SEQ ID NO: 825 - RNA, 273 nt, Oryza sativa
ugugagagaaugaagcugccagcaugaucugguugucaggcaugagccaaaucuauccaugguguuggugguacugaaauuaccgcguuuucgagguuuuucgucgugucaacuugcgaagggaauuacggguucuugaugagcauuggugauaggaggugugggcuugguuaguagagguagaauuaugauuguucuugugaguuucaguaagaggugggagugauuggaauuuggcuccaucagaucauguugcagcuucacucucucacc

SEQ ID NO: 826 - RNA, 113 nt, Oryza sativa
cacaaguggaugaagcugccagcaugaucugaucacaguaguucucuagcugaugaugauuuacaaaaccuagagacaugcaucagaucaucuggcaguuucaucuucucaug

SEQ ID NO: 827 - RNA, 82 nt, Oryza sativa
cauaagcaggugaagcugccagcaugaucugaaagcaucucaaaccagcgaucagaucauccggcagcuucaucuucucaug

SEQ ID NO: 828 - RNA, 120 nt, Oryza sativa
cacaaguuggugaagcugccagcaugaucugaugaugaugaugauccaccucucucaucuguguucuugauuaauuacggaucaaucgaucaggucaugcuguaguuucaucugcugguu

SEQ ID NO: 829 - RNA, 201 nt, Oryza sativa
ugugagaggcugaagcugccagcaugaucugguccaugaguugcacugcugaauauauugaauucagccaggagcugcuacugcaguucugaucucgaucugcauucguuguucugagcuauguauggauuugaucgguuugaaggcauccaugucuuuaauuucaucgaucagaucauguugcagcuucacucucucacu

SEQ ID NO: 830 - RNA, 87 nt, Oryza sativa
cgccucgggcucgcuuggugcagaucgggacccgccgccgccgcugccggggccggaucccgccuugcaccaagugaaucggagccg

SEQ ID NO: 831 - RNA, 106 nt, Oryza sativa
uggucuugugaggcuuggugcagcucgggaacuguucuugauggacuggcaggaacuccauguccaccacugccacuccuguguuguggcauuccuccuugccguu

SEQ ID NO: 832 - RNA, 115 nt, Oryza sativa
ggccauggugcagccaaggaugacuugccgaucgaucgaucuaucuaugaagcuaagcuagcuggccauggauccauccaucaauuggcaaguuguucuuggcuacaucuuggcc

SEQ ID NO: 833 - RNA, 128 nt, Oryza sativa
gaacggaaugcagccaaggaugacuugccgguacguguaugcauguuucaagguacuauaugugcccccaacuguuuuagauccaugcugacauuuuccggcaaguuguccuuggcuacgucuuguuc

SEQ ID NO: 834 - RNA, 112 nt, Oryza sativa
gaacgggaugcagccaaggaugacuugccggcuccugguauugggggaaucucagcuuugcugaagcgccuuggaguuagccggcaagucuguccuuggcuacaccuagcuc

SEQ ID NO: 835 - RNA, 103 nt, Oryza sativa
auuuaucguguagccaaggaugaauugccggcguuucacgcuguugauggugcgugcauauauaaguuggcgccggcaagucauuucaggcuacauguuugcc

SEQ ID NO: 836 - RNA, 132 nt, Oryza sativa
gcugguuguguagccaaggaugacuugccggccugauuuguguucaucagcaauccagcauaugcuguauugccgugugugaucgaucgaugcauggaccggcaaguuauuuucuuuggcuacauuacaacc

SEQ ID NO: 837 - RNA, 169 nt, Oryza sativa
gcugauucgguagccaaggaugacuugccuaaugccuaugugcauguguuuauacgcugcucaucugcauuuugauuauccccugaucaguccugucgucaauauauguguguguaguacucuguacucauacauauauaggcaugucuuccuuggcuauucggagcgg

SEQ ID NO: 838 - RNA, 126 nt, Oryza sativa
cugccucugguagccaaggaugacuugccuauugugcucuucugaaugaugcagugccaugaucaguguggccuggcugguucagaugagccgagauaggcagucuccuuggcuagccugaguggc

SEQ ID NO: 839 - RNA, 122 nt, Oryza sativa
uuagcucugguagccaaggaugacuugccuguguccuuguguguaaggaucauuaauuauuauucagaaaaugauccuuucagcagguuucaugggcagucuccuuggcuagccugagugau

SEQ ID NO: 840 - RNA, 108 nt, Oryza sativa
guagcucugguagccaaggaugacuugccuguguccuuguguagaggaucauucagaaaaugagccuugaacugguucauaggcagucuccuuggcuagucugagucg

SEQ ID NO: 841 - RNA, 125 nt, Oryza sativa
ucgcaucugguagccaaggaugacuugccugugucucugcucaugugcaguagaagaagaugcauuucuagcugcuuucugcauaugugaucucacaggcagucuccuuggcuagccugagcggc

SEQ ID NO: 842 - RNA, 156 nt, Oryza sativa
ucugucuagauagccaaggaugacuugccuguggccucuuggagagagagguguagcuuaauuagcagcaugguuugagcauugcuugaucgguugaucgcuucgcuugcucugcaugagaucuuacaggcagucuccuuggcuagucugggcggc

SEQ ID NO: 843 - RNA, 108 nt, Oryza sativa
uuaucucugauagccaaggaugacuugccuguguccucccugaaggauuagcaauuuaaugauccuuuaagcugguucaugggcagucuccuuggcuagccugagugg

SEQ ID NO: 844 - RNA, 138 nt, Oryza sativa
ugaguccugguagccaaggaugacuugccuguauaucuauauauauaugugugugugaucaauggauggauugaucaagcugcuugcaggcucaugcauauauauguacaggcagucuccuuggcuagcccggcuacc

SEQ ID NO: 845 - RNA, 177 nt, Oryza sativa
cucccuuugguagccaagaaugacuugccuaugcguuuugccuuguguuggcucauccauccgucuaucagccguugcagauuugcagugacagauuaaaggguuucugaaagaaauucuugugauggaugugcaauguggcugcaugggccggucuucuuggcuagccagaguggc

SEQ ID NO: 846 - RNA, 166 nt, Oryza sativa
cucccuuugguagccaagaaugacuugccuaugcuuuugcccucuguuggcucauccauccaucuaucuaucugccauggcagauuaaggguuuuugaaagaaauucuugugauaggaugugcaauguggcugcaugggccggucuucuuggcuagccagaguggc

SEQ ID NO: 847 - RNA, 126 nt, Oryza sativa
gagcaagguguagccaaggacaaacuugccggaucaacagagaaggacugccagucuccggccaauuaauuaaccucgccgucggccaucgccggccggcaagucauccuuggcugcauccugcuc

SEQ ID NO: 848 - RNA, 108 nt, Oryza sativa
ccacucaggcuagccaaggagacugcccaugaaccagcuuaaaggaucauuaaauugcuaauccuucagggaggacacaggcaagucauccuuggcuaucagagauaa

SEQ ID NO: 849 - RNA, 98 nt, Oryza sativa
uggaaagagcgauauuggugagguucaauccgaugauugguuuuacagcagugguaaaaucaguaucugauugagccgcgccaauaucucuuccucuc

SEQ ID NO: 850 - RNA, 100 nt, Oryza sativa
gcgacgacgggauauuggggcgguucaaucagaaagcuugugcuccagaagcgaggagcucuacucuuuugauugagccgugccaauaucacgucgcauc

SEQ ID NO: 851 - RNA, 99 nt, Oryza sativa
gugggaacgggauauuggugcgguucaaucagaaagcuugugcuccgaaggcgaggggcuccacucuuugauugagccgugccaauaucacgucgccuu

SEQ ID NO: 852 - RNA, 135 nt, Oryza sativa
uuguagcuaugauguuggcccggcucacucagauggaucaucggugcagaagagugcaugaaucugaugcagucucaguguaguaugcuccaugcuggaacuucugauugagccgugccaauaucucagcaccau

SEQ ID NO: 853 - RNA, 119 nt, Oryza sativa
ugguagcuaugauguuggcucggcucacucagacggcauuggcgugaugcaaagcaugcaugcgugcucgcuagcucacuuguguuucugauugagccgugccaauaucuuagugcucu

SEQ ID NO: 854 - RNA, 127 nt, Oryza sativa
gggagagugcgauguuggcaugguucaaucaaaccgggcaaacuuaugcacuagcuaagcaagaugcagggauacgcaguaugguuuuguuuggucugauugagccgugccaauaucacaagcuugc

SEQ ID NO: 855 - RNA, 89 nt, Oryza sativa
gacauggcaugguauugacuuggcucaucucagcaacagcaaacugcaugcagcgcuggaggugagccgagccaauaucacuucauguc

SEQ ID NO: 856 - RNA, 109 nt, Oryza sativa
guguuugcgggcguggcaucaucaagauucacauccaugcauauaucacaagacgcacauauacauccgauuuggcugagaaucuugaugaugcugcauccgcagacaa

SEQ ID NO: 857 - RNA, 238 nt, Oryza sativa
gugauuucugacguggcaucaucaagauucacacauuacauugcaugcaugcauaugucuaugcaucuuugagcuuguuguucugaucucaacaaccuagcuagcuaauauuucucuccuggcccugaccugcaugaugcaugguugcacgcauggagagagaagagagagaucgaagcuaauuaagcgcauguguauauaugugugggaaucuugaugaugcugcaucggaaauuaa

SEQ ID NO: 858 - RNA, 111 nt, Oryza sativa
cuuguugcgggugcagcgucaucaagauucacgugugccgcacggcacacguaucgguuuucaaguguagucaucgugcgugaaucuugaugaugcugcaccagcaaagag

SEQ ID NO: 859 - RNA, 115 nt, Oryza sativa
uggggaagcauccaaagggaucgcauugauccuucaucgcucucgcucgcuuccauggcggucgucgucuacaagcagcuugacggaucaugcgauccuuuuggaggcuuccucu

SEQ ID NO: 860 - RNA, 110 nt, Oryza sativa
uacugagaguucuuuggcauucuguccaccuccuugucgaauccucagagacagaaaucucauaucuguugaucuuggaggugggcauacugccaauggagcuguguagg

SEQ ID NO: 861 - RNA, 142 nt, Oryza sativa
uuguccacuggaguucuccucaauccacuucaguagauagcuauggcuaggccucauugcauugcacuguuacauaacugugaucauggggccaaaagcuagcuauguauagugaagugcuugggggaacuccaguugacac

SEQ ID NO: 862 - RNA, 86 nt, Oryza sativa
gagucccuaggaguuccuuucaagcacuuuacgacacacuguauugagaguugucgugaaguguuugggggaacucuuagugucgc

SEQ ID NO: 863 - RNA, 89 nt, Oryza sativa
guauuaucaagaguucucuuuaagcacuucauacgacaccauuauuuauaggguuguugugaaguguuuggaggaacucucggugucuu

SEQ ID NO: 864 - RNA, 88 nt, Oryza sativa
guauugucgugaguucccuucaagcacuucacguggcacuaucucaaugccuacuaugugaaguguuugggggaacucucgguaucac

SEQ ID NO: 865 - RNA, 88 nt, Oryza sativa
guauuaucgagaguucccuucaaccacuucacguggcacuguuucaaggccuauugugugaaguguuugggggaacucucgauaucac

SEQ ID NO: 866 - RNA, 88 nt, Oryza sativa
guauuaucgcggguucccuucaaucacuucacaugguacuauuucaaggccuacuaugugaaguauuugggggaacucucgaugucac

SEQ ID NO: 867 - RNA, 86 nt, Oryza sativa
guaucaccgugaguucccuucgaacacuucacguggcacuauuucaaugccuauugugaaguguuugggggaacucucgaugucau

SEQ ID NO: 868 - RNA, 112 nt, Oryza sativa
uuguuaccuggaguuuccucaacacacuucacaucugcuaggcccuauuacaauugcgcaauguggggucugcaauugguagugaaguguuugggggaacucuagguggcac

SEQ ID NO: 869 - RNA, 99 nt, Oryza sativa
guuuuaccgggaguucccuacaagcacuucacguagagcuuucuauugacauggagcuuuagaacaaugugaaguguuugggggaacucuugguaccaa

SEQ ID NO: 870 - RNA, 112 nt, Oryza sativa
guguucccaagaguuccuugcaagcacuucacauagaacuucuguuacucucauguaacauugggaacuugagaagcuacugugaaguguuugggggaacucuagguggcac

SEQ ID NO: 871 - RNA, 99 nt, Oryza sativa
guuuuaucgggaguuuccuucaagcacuucacguagagcuuucuauugauauggagcuuuggaacaaugugaaguguuugggggaacucuugauaccaa

SEQ ID NO: 872 - RNA, 112 nt, Oryza sativa
guggccccaggaguuccuugcaagcacuucacauagaacuucaguuacucucauacaacauugugauuuugagaagcuauugugaaguguuugggggaacucucggugccaa

SEQ ID NO: 873 - RNA, 114 nt, Oryza sativa
augucccuaagcguuccuuccaagcacuucacacagagcuuuuauuucucucacaucgauugagaacuuaauuagaagcuuuugugaaguguuugggggaacucuuggugccac

SEQ ID NO: 874 - RNA, 86 nt, Oryza sativa
guaucaccgugaguucccuucaagcacuucacguggcacuauuucaaugccuauugugaaguguuugggggaacucucgauguucc

SEQ ID NO: 875 - RNA, 143 nt, Oryza sativa
uuauccacuggaguucuccucaaaccacuucagcagauagcuagcuaggccucauugcauugcaccacuguugcauaacuaugagcauggggccaaaaguuagcugcuuauagugaaguguuuggggaaacuccgguuggcaa

SEQ ID NO: 876 - RNA, 86 nt, Oryza sativa
acacugccaggaauucccuucaagcaauucaugaaacaauauuuugagaguuguugugaagcguuugggggaaaucucagugucgc

SEQ ID NO: 877 - RNA, 143 nt, Oryza sativa; identical to SEQ ID NO: 875

SEQ ID NO: 878 - RNA, 143 nt, Oryza sativa; identical to SEQ ID NO: 875

SEQ ID NO: 879 - RNA, 86 nt, Oryza sativa; identical to SEQ ID NO: 876

SEQ ID NO: 880 - RNA, 154 nt, Oryza sativa
cuuugugaucuuccacagcuuucuugaacugcacgcaugaugaauaaucccuuugguuaauugugaucuggucucugagagaucguagcuagacucgaucgguugcauuggcaucagagagagcaguucaauaaagcugugggaaauugcagag

SEQ ID NO: 881 - RNA, 116 nt, Oryza sativa
cuuuguggucuuccacagcuuucuugaacugcaucuuugagagagauuagcaucccuauguguggauuuugcuugcacgagugugcaguucaauaaagcugugggaaauuacagag

SEQ ID NO: 882 - RNA, 141 nt, Oryza sativa
ugccaugccuuuccacagcuuucuugaacuucucuugugccucacucacuuucauuacuggagagauaugcaucaucaguggaagcuuauagggagaggaguacaagaagagggucaagaaagcugugggaagaaauggca

SEQ ID NO: 883 - RNA, 114 nt, Oryza sativa
aucaaaugcaucauugagugcagcguugaugaacaacgguaaccgguccauguugaugcgcauuuggccggugaucugaucaucaucagcgcuucacucaaucaugcguuuggc

SEQ ID NO: 884 - RNA, 118 nt, Oryza sativa
agggaaggcauuauugagugcagcguugaugaaccugccggccggcuaaauuaauuagcaagaaagucugaaacuggcucaaagguucaccagcacugcacccaaucacgccuuugcu

SEQ ID NO: 885 - RNA, 115 nt, Oryza sativa
gcugaacccagaggagugguacugagaacacaggugccaauacaauguauggugagcuacuguauaauggaguaauucuguaacuguguucucaggucaccccuuuggguuucuu

SEQ ID NO: 886 - RNA, 88 nt, Oryza sativa
ggaguuccuacaggggcgagcugggaacacacggugaugaggcggucuggucuuucguguguucucaggucgccccugccgggacucu

SEQ ID NO: 887 - RNA, 149 nt, Oryza sativa
cugugaauuacagggcaguucaccuuuggcacaagggcaagcaguagaaaccaugcgugcuugcuagagcuggaaaugaugcugguagcauugcaugguucagggaucacagaucucgugccaaaggagaauugcccugcgauuuuguc

SEQ ID NO: 888 - RNA, 97 nt, Oryza sativa
gugagaaucacagugcgauucuccucuggcauggcaugagaggccuaaaaaagagacgcacugccgugccaaaggagaauugcccugccauucagaa

SEQ ID NO: 889 - RNA, 110 nt, Oryza sativa
cggcgaauuacagggcgguuucuccuuuggcacguacggaggcaaggcaugcggugaaaaaucucuagcuagccaugcgugccaaaggagaauugcccugcgauucacca

SEQ ID NO: 890 - RNA, 286 nt, Oryza sativa
aagacaguaguaggcagcucuccucuggcaggugcauucuaggugauuuuguaauuguauaugcauccaagguauauacaguccggccauggugcuacauugcaaucauccauaugugauugcauuguguauauauauacaugguggccuuugauagaccaucauauaucgguugguuaugugcauguauguauauaccagcugcuacuagcuuugaucgaucgccauguagcgauugaauucaccaaaacggccugccaaaggagaguugcccugcgacugucuu

SEQ ID NO: 891 - RNA, 118 nt, Oryza sativa
acaugcauuaccgggugagucuuccuuggcaguguucgaaucggcaguaccggucugcaagugaucggucaaucaccaguucaccacugccaaaggagauuugcccagcaaugcaacu

SEQ ID NO: 892 - RNA, 117 nt, Oryza sativa
ugguggauuaccgggccaugucuccuugggcagaggugaucagauugcacacuucacuucaaccucuugcucuagcuuguucucucugccaaaggagauuugcccagcaauccacau

SEQ ID NO: 893 - RNA, 118 nt, Oryza sativa
augugcauugcagggcaacuacuccauuggcgagggauggauuggauauggauauggcugaugcuuccauuugaucccaucccuaucugccaaaggagauuugcccggcgauucacuc

SEQ ID NO: 894 - RNA, 100 nt, Oryza sativa
ccaugcauuacugggcaggucucccuuggcaguggccgaucgagcugaucaaaaccacgcaaaagccacugccaaaggagacuugcccagcaaugcagau

SEQ ID NO: 895 - RNA, 116 nt, Oryza sativa
gugagaaucacagugcaguucuccucuggcauggagggcaagaggagcugaauagcuaauggaugauaaauugcuagccuuucccugccaaaggagagcugcccugccauucagug

SEQ ID NO: 896 - RNA, 106 nt, Oryza sativa
aguccaguuucagggcuccucucucuuggcagggagcaugugaagucuuuuguagcucacucauuuucagcccucugccaaaggagaguugcccuaaaacuggacu

SEQ ID NO: 897 - RNA, 106 nt, Oryza sativa
agcugcauugcugggcaaguuguccuuuggcagauguugcaguucaucaucgaugccuggggguuaccagacuacugccaaaggaaauuugccccggaauucaucu

SEQ ID NO: 898 - RNA, 21 nt, Brassica
ugacagaagagagugagcaca

SEQ ID NO: 899 - RNA, 21 nt, Glycine
uugacagaagauagagagcac

SEQ ID NO: 900 - RNA, 21 nt, Glycine
uugacagaagauagagagcac

SEQ ID NO: 901 - RNA, 21 nt, Glycine
ugacagaagagagugagcaca

SEQ ID NO: 902 - RNA, 21 nt, Glycine
ugacagaagagagagagcaca

SEQ ID NO: 903 - RNA, 21 nt, Helianthus
uugacagaagauagagagcac

SEQ ID NO: 904 - RNA, 21 nt, Lotus
uugacagaagagagagagcac

SEQ ID NO: 905 - RNA, 21 nt, Medicago
uugacagaagauagagggcac

SEQ ID NO: 906 - RNA, 21 nt, Nicotiana
uugacagaagauagagagcac

SEQ ID NO: 907 - RNA, 21 nt, Oryza
ugacagaagagagugagcaca

SEQ ID NO: 908 - RNA, 21 nt, Oryza
ugacagaagagagugagcaca

SEQ ID NO: 909 - RNA, 21 nt, Saccharum
ugacagaagagagugagcaca

SEQ ID NO: 910 - RNA, 21 nt, Saccharum
ugacagaagagagugagcaca

SEQ ID NO: 911 - RNA, 21 nt, Sesamum
uugacagaagagagagagcac

SEQ ID NO: 912 - RNA, 21 nt, Solanum
uugacagaagauagagagcac

SEQ ID NO: 913 - RNA, 21 nt, Solanum
ugacagaagagagugagcaca

SEQ ID NO: 914 - RNA, 21 nt, Sorghum
ugacagaagagagugagcaca

SEQ ID NO: 915 - RNA, 21 nt, Vitis
ugacagaagagagagagcaug

SEQ ID NO: 916 - RNA, 21 nt, Zea
ugacagaagagagugggcaca

SEQ ID NO: 917 - RNA, 21 nt, Zea
ugacagaagagagugagcaca

SEQ ID NO: 918 - RNA, 21 nt, Zea
ugacagaagagagugagcaca

SEQ ID NO: 919 - RNA, 21 nt, Zea
ugacagaagagagugagcaca

SEQ ID NO: 920 - RNA, 21 nt, Zea
ugacagaagagagugagcaca

SEQ ID NO: 921 - RNA, 21 nt, Zea
ugacagaagagagugagcaca

SEQ ID NO: 922 - RNA, 20 nt, Glycine
uuggacugaagggagcuccc

SEQ ID NO: 923 - RNA, 20 nt, Glycine
uuggacugaaaggagcuccu

SEQ ID NO: 924 - RNA, 21 nt, Glycine
uuuggauugaagggagcucua

SEQ ID NO: 925 - RNA, 21 nt, Glycine
auuggagugaagggagcucca

SEQ ID NO: 926 - RNA, 20 nt, Glycine
uuggacugaagggagcuccc

SEQ ID NO: 927 - RNA, 21 nt, Hordeum
uuuggauugaagggagcucug

SEQ ID NO: 928 - RNA, 20 nt, Oryza
uuggacugaagggagcuccc

SEQ ID NO: 929 - RNA, 21 nt, Medicago
uuuggauugaagggagcucua

SEQ ID NO: 930 - RNA, 21 nt, Oryza
uuuggauugaagggagcucug

SEQ ID NO: 931 - RNA, 21 nt, Oryza
uuuggauugaagggagcucug

SEQ ID NO: 932 - RNA, 21 nt, Pennisetum
uuuggauugaagggagcucug

SEQ ID NO: 933 - RNA, 20 nt, Physcomitrella
uuggacugaagggagcucca

SEQ ID NO: 934 - RNA, 21 nt, Saccharum
uuuggauugaagggagcucug

SEQ ID NO: 935 - RNA, 21 nt, Saccharum
uuuggauugaagggagcucug

SEQ ID NO: 936 - RNA, 21 nt, Saccharum
uuuggauugaagggagcucug

SEQ ID NO: 937 - RNA, 20 nt, Saccharum
uuggaucgaagggagcucuu

SEQ ID NO: 938 - RNA, 21 nt, Saccharum
cuuggauugaagggagcuccu

SEQ ID NO: 939 - RNA, 21 nt, Saccharum
uuuggauugaaaggagcucuu

SEQ ID NO: 940 - RNA, 21 nt, Saccharum
uuuggauugaagggagcucug

SEQ ID NO: 941 - RNA, 21 nt, Sorghum
uuuggauugaagggagcucug

SEQ ID NO: 942 - RNA, 21 nt, Sorghum
uuuggauugaagggagcucug

SEQ ID NO: 943 - RNA, 21 nt, Sorghum
uuuggauugaagggagcucug

SEQ ID NO: 944 - RNA, 20 nt, Triticum
uuggacugaagggagcuccc

SEQ ID NO: 945 - RNA, 21 nt, Triticum
uuuggauugaagggagcucug

SEQ ID NO: 946 - RNA, 21 nt, Triticum
uuuggauugaagggagcucug

SEQ ID NO: 947 - RNA, 21 nt, Vitis
uuuggauugaagggagcucua

SEQ ID NO: 948 - RNA, 21 nt, Vitis
uuuggacugaagggagcuccu

SEQ ID NO: 949 - RNA, 21 nt, Vitis
uuuggauugaagggagcucua

SEQ ID NO: 950 - RNA, 21 nt, Zea
cuuggauugaagggagcuccu

SEQ ID NO: 951 - RNA, 20 nt, Zea
uuggaucgaagggagcucuu

SEQ ID NO: 952 - RNA, 21 nt, Glycine
ugccuggcucccuguaugcca

SEQ ID NO: 953 - RNA, 21 nt, Oryza
ugccuggcucccuguaugcca

SEQ ID NO: 954 - RNA, 21 nt, Oryza
ugccuggcucccuguaugcca

SEQ ID NO: 955 - RNA, 21 nt, Triticum
ugccuggcucccuguaugcca

SEQ ID NO: 956 - RNA, 21 nt, Zea
ugccuggcucccuguaugcca

SEQ ID NO: 957 - RNA, 21 nt, Lupinus
ucgauaaaccucugcauccag

SEQ ID NO: 958 - RNA, 21 nt, Medicago
ucgauaaaccucugcauccag

SEQ ID NO: 959 - RNA, 21 nt, Oryza
ucgauaagccucugcauccag

SEQ ID NO: 960 - RNA, 21 nt, Vitis
ucgauaaaccucugcauccag

SEQ ID NO: 961 - RNA, 21 nt, Populus
uggagaagcagggcacgugca

SEQ ID NO: 962 - RNA, 21 nt, Populus
uggagaagcagggcacgugca

SEQ ID NO: 963 - RNA, 21 nt, Triticum
uggagaagcagggcacgugca

SEQ ID NO: 964 - RNA, 21 nt, Glycine
ucggaccaggcuucauucccu

SEQ ID NO: 965 - RNA, 21 nt, Glycine
ucggaccaggcuucauucccc

SEQ ID NO: 966 - RNA, 21 nt, Glycine
ucggaccaggcuucauucccg

SEQ ID NO: 967 - RNA, 21 nt, Glycine
ucggaccaggcuucauucccc

SEQ ID NO: 968 - RNA, 21 nt, Glycine
ucggaccaggcuucauucccc

SEQ ID NO: 969 - RNA, 21 nt, Hedyotis
ucggaccaggcuucauccccc

SEQ ID NO: 970 - RNA, 21 nt, Hordeum
ucggaccaggcuucauucccc

SEQ ID NO: 971 - RNA, 21 nt, Ipomoea
ucggaccaggcuucauuccuc

SEQ ID NO: 972 - RNA, 21 nt, Medicago
ucggaccaggcuucauucccc

SEQ ID NO: 973 - RNA, 21 nt, Medicago
ucggaccaggcuucauucccc

SEQ ID NO: 974 - RNA, 21 nt, Medicago
ucggaccaggcuucauucccc

SEQ ID NO: 975 - RNA, 21 nt, Medicago
ucggaccaggcuucauuccuc

SEQ ID NO: 976 - RNA, 21 nt, Oryza
ucggaccaggcuucaaucccu

SEQ ID NO: 977 - RNA, 21 nt, Sorghum
ucggaccaggcuucauucccc

SEQ ID NO: 978 - RNA, 21 nt, Zea
ucggaccaggcuucauucccc

SEQ ID NO: 979 - RNA, 21 nt, Glycine
ugaagcugccagcaugaucug

SEQ ID NO: 980 - RNA, 21 nt, Glycine
ugaagcugccagcaugaucua

SEQ ID NO: 981 - RNA, 21 nt, Oryza
ugaagcugccagcaugaucug

SEQ ID NO: 982 - RNA, 21 nt, Oryza
ugaagcugccagcaugaucug

SEQ ID NO: 983 - RNA, 21 nt, Phaseolus
ugaagcugccagcaugaucuu

SEQ ID NO: 984 - RNA, 21 nt, Saccharum
ugaagcugccagcaugaucug

SEQ ID NO: 985 - RNA, 21 nt, Saccharum
ugaagcugccagcaugaucug

SEQ ID NO: 986 - RNA, 21 nt, Saccharum
ugaagcugccagcaugaucug

SEQ ID NO: 987 - RNA, 21 nt, Zea
ugaagcugccagcaugaucug

SEQ ID NO: 988 - RNA, 21 nt, Arabidopsis
ucgcuuggugcaggucgggaa

SEQ ID NO: 989 - RNA, 21 nt, Betula
ucgcuuggugcaggucgggaa

SEQ ID NO: 990 - RNA, 21 nt, Glycine
ucgcuuggugcaggucgggaa

SEQ ID NO: 991 - RNA, 21 nt, Hedyotis
ucgcuuggugcaggucgggaa

SEQ ID NO: 992 - RNA, 21 nt, Lycopersicon
ucgcuuggugcaggucgggac

SEQ ID NO: 993 - RNA, 21 nt, Lycopersicon
ucgcuuggugcaggucgggac

SEQ ID NO: 994 - RNA, 21 nt, Oryza
ucgcuuggugcagaucgggac

SEQ ID NO: 995 - RNA, 21 nt, Populus
ucgcuuggugcaggucgggaa

SEQ ID NO: 996 - RNA, 21 nt, Populus
ucgcuuggugcaggucgggaa

SEQ ID NO: 997 - RNA, 21 nt, Saccharum
ucgcuuggugcagaucgggac

SEQ ID NO: 998 - RNA, 21 nt, Saccharum
ucgcuuggugcagaucgggac

SEQ ID NO: 999 - RNA, 21 nt, Solanum
ucgcuuggugcaggucgggac

SEQ ID NO: 1000 - RNA, 21 nt, Solanum
ucgcuuggugcaggucgggac

SEQ ID NO: 1001 - RNA, 21 nt, Solanum
ucgcuuggugcaggucgggac

SEQ ID NO: 1002 - RNA, 21 nt, Sorghum
ucgcuuggugcagaucgggac

SEQ ID NO: 1003 - RNA, 21 nt, Vitis
ucgcuuggugcaggucgggaa

SEQ ID NO: 1004 - RNA, 21 nt, Zea
ucgcuuggugcagaucgggac

SEQ ID NO: 1005 - RNA, 21 nt, Zea
ucgcuuggugcagaucgggac

SEQ ID NO: 1006 - RNA, 21 nt, Zea
ucgcuuggugcagaucgggac

SEQ ID NO: 1007 - RNA, 21 nt, Glycine
cagccaaggaugacuugccgg

SEQ ID NO: 1008 - RNA, 21 nt, Glycine
cagccaaggaugacuugccgg

SEQ ID NO: 1009 - RNA, 21 nt, Glycine
cagccaaggaugacuugccgg

SEQ ID NO: 1010 - RNA, 21 nt, Oryza
cagccaaggaugacuugccga

SEQ ID NO: 1011 - RNA, 21 nt, Oryza
cagccaaggaugacuugccgg

SEQ ID NO: 1012 - RNA, 21 nt, Oryza
uagccaaggagacugccuaug

SEQ ID NO: 1013 - RNA, 21 nt, Oryza
cagccaaggaugacuugccga

SEQ ID NO: 1014 - RNA, 21 nt, Oryza
uagccaaggagacugcccaug

SEQ ID NO: 1015 - RNA, 21 nt, Oryza
uagccaaggagacugccuauc

SEQ ID NO: 1016 - RNA, 21 nt, Oryza
uagccaagaauggcuugccua

SEQ ID NO: 1017 - RNA, 21 nt, Populus
cagccaaggaugacuugccga

SEQ ID NO: 1018 - RNA, 21 nt, Populus
cagccaaggaugauuugccga

SEQ ID NO: 1019 - RNA, 21 nt, Triticum
uagccaaggaugauuugccug

SEQ ID NO: 1020 - RNA, 21 nt, Zea
uagccaaggacagacuugccg

SEQ ID NO: 1021 - RNA, 21 nt, Arabidopsis
ugauugagccgcgccaauauc

SEQ ID NO: 1022 - RNA, 21 nt, Glycine
ucauugagccgugccaauauc

SEQ ID NO: 1023 - RNA, 21 nt, Hedyotis
cgauugagccgugccaauauc

SEQ ID NO: 1024 - RNA, 21 nt, Hordeum
ugauugagccgugccaauauc

SEQ ID NO: 1025 - RNA, 21 nt, Oryza
ugauugagccgugccaauauc

SEQ ID NO: 1026 - RNA, 21 nt, Oryza
ugauugagccgugccaauauc

SEQ ID NO: 1027 - RNA, 21 nt, Triticum
cgauugagccgugccaauauc

SEQ ID NO: 1028 - RNA, 21 nt, Triticum
ugauugagccgugccaauauc

SEQ ID NO: 1029 - RNA, 21 nt, Zea
ugauugagccgugccaauauc

SEQ ID NO: 1030 - RNA, 21 nt, Zea
ugauugagccgugccaauauc

SEQ ID NO: 1031 - RNA, 21 nt, Citrus
agaaucuugaugaugcugcaa

SEQ ID NO: 1032 - RNA, 21 nt, Glycine
agaaucuugaugaugcugcau

SEQ ID NO: 1033 - RNA, 21 nt, Glycine
agaaucuugaugaugcugcau

SEQ ID NO: 1034 - RNA, 21 nt, Lycopersicon
agaaucuugaugaugcugcau

SEQ ID NO: 1035 - RNA, 21 nt, Solanum
agaaucuugaugaugcugcau

SEQ ID NO: 1036 - RNA, 21 nt, Oryza
uccaaagggaucgcauugauc

SEQ ID NO: 1037 - RNA, 21 nt, Oryza
uccaaagggaucgcauugauc

SEQ ID NO: 1038 - RNA, 21 nt, Populus
uccaaagggaucgcauugauc

SEQ ID NO: 1039 - RNA, 20 nt, Glycine
uuggcauucuguccaccucc

SEQ ID NO: 1040 - RNA, 20 nt, Glycine
uuggcauucuguccaccucc

SEQ ID NO: 1041 - RNA, 20 nt, Robinia
uuggcauucuguccaccucc

SEQ ID NO: 1042 - RNA, 21 nt, Glycine
ugaaguguuugggggaacucu

SEQ ID NO: 1043 - RNA, 21 nt, Oryza
gugaaguguuugggggaacuc

SEQ ID NO: 1044 - RNA, 21 nt, Oryza
gugaaguguuugggggaacuc

SEQ ID NO: 1045 - RNA, 21 nt, Oryza
augaaguguuuggaggaacuc

SEQ ID NO: 1046 - RNA, 21 nt, Oryza
gugaagugcuugggggaacuc

SEQ ID NO: 1047 - RNA, 21 nt, Oryza
gugaaguguuugggggaacuc

SEQ ID NO: 1048 - RNA, 21 nt, Oryza
gugaaguguuuggaggaacuc

SEQ ID NO: 1049 - RNA, 21 nt, Oryza
gugaaguguuugggggaacuc

SEQ ID NO: 1050 - RNA, 21 nt, Triticum
gugaaguguuuggagggacuc

SEQ ID NO: 1051 - RNA, 21 nt, Triticum
augaaguguuugggggaacuc

SEQ ID NO: 1052 - RNA, 21 nt, Brassica
uuccacagcuuucuugaacuu

SEQ ID NO: 1053 - RNA, 21 nt, Glycine
uuccacagcuuucuugaacuu

SEQ ID NO: 1054 - RNA, 21 nt, Glycine
uuccacagcuuucuugaacug

SEQ ID NO: 1055 - RNA, 21 nt, Mesembryanthemum
uuccacagcuuucuugaacug

SEQ ID NO: 1056 - RNA, 21 nt, Mesembryanthemum
uuccacagcuuucuugaacug

SEQ ID NO: 1057 - RNA, 21 nt, Mesembryanthemum
uuccucagcuuucuugaacug

SEQ ID NO: 1058 - RNA, 21 nt, Mesembryanthemum
ucccacagcuuucuugaacuu

SEQ ID NO: 1059 - RNA, 21 nt, Oryza
uuccacagcuuucuugaacug

SEQ ID NO: 1060 - RNA, 21 nt, Populus
uuccacagcuuucuugaacug

SEQ ID NO: 1061 - RNA, 21 nt, Populus
uuccacagcuuucuugaacug

SEQ ID NO: 1062 - RNA, 21 nt, Prunus
uuccacagcuuucuugaacug

SEQ ID NO: 1063 - RNA, 21 nt, Prunus
uuccacagcuuucuugaacug

SEQ ID NO: 1064 - RNA, 21 nt, Saccharum
uuccacagcuuucuugaacug

SEQ ID NO: 1065 - RNA, 21 nt, Solanum
uuccacagcuuucuugaacuu

SEQ ID NO: 1066 - RNA, 21 nt, Zea
uuccacagcuuucuugaacug

SEQ ID NO: 1067 - RNA, 21 nt, Zea
uuccacagcuuucuugaacug

SEQ ID NO: 1068 - RNA, 20 nt, Hordeum
cguugagugcagcguugaug

SEQ ID NO: 1069 - RNA, 20 nt, Hordeum
cguugagugcagcguugaug

SEQ ID NO: 1070 - RNA, 21 nt, Citrus
uguguucucaggucaccccuu

SEQ ID NO: 1071 - RNA, 21 nt, Glycine
uguguucucaggucgccccug

SEQ ID NO: 1072 - RNA, 21 nt, Glycine
uguguucucaggucaccccuu

SEQ ID NO: 1073 - RNA, 21 nt, Glycine
uguguucucaggucaccccuu

SEQ ID NO: 1074 - RNA, 21 nt, Helianthus
cguguucucaggucgccccug

SEQ ID NO: 1075 - RNA, 21 nt, Lactuca
uguguucucaggucgccccug

SEQ ID NO: 1076 - RNA, 21 nt, Lactuca
uguguucucaggucgccccug

SEQ ID NO: 1077 - RNA, 21 nt, Lotus
uguguucucaggucaccccuu

SEQ ID NO: 1078 - RNA, 21 nt, Medicago
uguguucucaggucaccccuu

SEQ ID NO: 1079 - RNA, 21 nt, Medicago
uguguucucaggucgccccug

SEQ ID NO: 1080 - RNA, 21 nt, Nicotiana
uguguucucaggucgccccug

SEQ ID NO: 1081 - RNA, 21 nt, Oryza
uguguucucaggucgccccug

SEQ ID NO: 1082 - RNA, 21 nt, Zea
uguguucucaggucgcccccg

SEQ ID NO: 1083 - RNA, 21 nt, Medicago
ugccaaaggagaguugcccug

SEQ ID NO: 1084 - RNA, 21 nt, Populus
ugccaagggagaauugcccug

SEQ ID NO: 1085 - RNA, 99 nt, Brassica
cauagcaacugacagaagagagugagcacacaaaaguaaucugcauauacugcauuugcuucucuugcgugcucacugcucuuucugucagauucuagu

SEQ ID NO: 1086 - RNA, 130 nt, Glycine
uuaagguuguugacagaagauagagagcacagaugaugauaugcauauuauauaauauauagcagggaacucaugaugaauugugcaucuuacuccuuugugcucucuauacuucugucaucaccuucag

SEQ ID NO: 1087 - RNA, 103 nt, Glycine
gugaugcuguugacagaagauagagagcacugaugaugaaaugcaugaaagggaauggcaucucacuccuuugugcucucuagucuucugucaucauccuucu

SEQ ID NO: 1088 - RNA, 101 nt, Glycine
gagagaggcugacagaagagagugagcacaugcuagugguauuuguaugagggcauacaauugcgggugcgugcucacuucucuaucugucagcuucccau

SEQ ID NO: 1089 - RNA, 101 nt, Glycine
aucucauguugacagaagagagagagcacaacccgggaauggcuaaaggagucuuugccuuuguugggagugugcccucucuuccucugucaucaucacau

SEQ ID NO: 1090 - RNA, 104 nt, Helianthus
ugauggauguugacagaagauagagagcacagagaagcaugaauugcacauagauauugcaauucacuccuucgugcucucuaugcuucugucauuaccuauua

SEQ ID NO: 1091 - RNA, 106 nt, Lotus
uucaugcauguugacagaagagagagagcacaacccaggaauggugaaagagagucuuugcuuuuguugggagugugcucucccuucuucugucaucaucacauga

SEQ ID NO: 1092 - RNA, 129 nt, Medicago
guaagguuguugacagaagauagagggcacuaaggaugauaugcauacacauauauauacaacauggaggaggagcuuaauugcauuucauuuccuuugugcucucuagacuucugucaucaccucauc

SEQ ID NO: 1093 - RNA, 151 nt, Nicotiana
ugugagauuguugacagaagauagagagcacagaugaugaucaugucugcuaaaucugggauuggagagggcacugaaucaauuaaacugcagagaauaaaaagcaucucaauucauuugugcucucuaugcuuccgucaucaccuucacc

SEQ ID NO: 1094 - RNA, 105 nt, Oryza
  misc_feature (7)..(7): n is a, c, g, or u
ugggagnucugacagaagagagugagcacacacggugcuuucuuagcaugcaagagccnaugcugggagcugugcgugcucacucucuaucugucagcccguuca

SEQ ID NO: 1095 - RNA, 106 nt, Oryza; identical to SEQ ID NO: 783

SEQ ID NO: 1096 - RNA, 116 nt, Saccharum
gguggaggcugacagaagagagugagcacacauggugccuuucuugcaugaugaacgaucgagagguucaugcucgaagcuaugcgugcucacuucucucucugucagccguuaga

SEQ ID NO: 1097 - RNA, 104 nt, Saccharum
  misc_feature (47)..(47): n is a, c, g, or u
uuugaagguuugacagaagagagugagcacacacggugguuucuuancaugagugccaugcugggagcugugcgugcucacucucuaucugucagccacucauc

SEQ ID NO: 1098 - RNA, 101 nt, Sesamum
auuaauuuguugacagaagagagagagcacagcccgccauugacaaagaggucuuugccuuuugugggauugugcucucuugcuucugccaacgaccgucu

SEQ ID NO: 1099 - RNA, 109 nt, Solanum
ugauaauuguugacagaagauagagagcacuaaugaugauaugcuaauuucauucagcaaaagcaucucacuucauuugugcucucuaugcuucugucaucaccuucgc

SEQ ID NO: 1100 - RNA, 104 nt, Solanum
aaucaagacugacagaagagagugagcacacgcagucgaauuguauaaacauuuauacaauugucauuugcgugugcucacuucucauucugucagcucucuca

SEQ ID NO: 1101 - RNA, 104 nt, Sorghum
cuugagagauugacagaagagagugagcacacggcgcgacgaacggcauaauauguaugucguccucgccgcgugcucacuucucuuucggucagccucuucug

SEQ ID NO: 1102 - RNA, 226 nt, Vitis
ugccucacaaugacagaagagagagagcaugcuggugggaaaacaauuacaaccuuugcucaucugaucuggaaaugcuuguaagcggcauucucuuggauuguaaucugaauucugccucuaucaucaaccugcccacaaacgaguuccuucagcugagugccuuuccggcuugagccuucugcaugaucagcugaguucuuucugcgccuuucauuguguccug

SEQ ID NO: 1103 - RNA, 119 nt, Zea
aggugaaagcugacagaagagagugggcacacauggugccuuucuugcaugauguaugaucgagagaguucaugcucgaagcuaugcgugcucacuucucucucugucagccauuagaa

SEQ ID NO: 1104 - RNA, 102 nt, Zea
uugaagguuugacagaagagagugagcacacacggugguuucuuaccaugagugucaugcuaggagcugugcgugcucacucucuaucugucagccacucau

SEQ ID NO: 1105 - RNA, 119 nt, Zea
aggugaaagcugacagaagagagugagcacacauggugccuuucuugcaugauguaugaucgagagaguucaugcucgaagcuaugcgugcucacuucucucucugucagccauuagaa

SEQ ID NO: 1106 - RNA, 102 nt, Zea
uugaagguuugacagaagagagugagcacacacggugguuucuuaccaugagugucaugcuaggagcugugcgugcucacucucuaucugucaaccacucau

SEQ ID NO: 1107 - RNA, 104 nt, Zea
uuugaagguuugacagaagagagugagcacacacggugguuccuuaccaugagugucaugcuaggagcugugcgugcucacucucuaucugucagccacucauc

SEQ ID NO: 1108 - RNA, 102 nt, Zea
ucgagagauugacagaagagagugagcacacggcgcgacgaacgacauagcauguaugccguccucgccgcgugcucacuucucuuucugucagccucuuuc

SEQ ID NO: 1109 - RNA, 189 nt, Glycine
gcgacgguaagagagcuuucuucaguccacuuaugggugacaauaagauuucaauuagcugccgacucauucauccaaaugcugagugaaagcgaagaaagauacucagcaaaugagugaaugaugcgggagacaaauugauucuuaaguguccuguacuuggacugaagggagcucccuuuuucuuuu

SEQ ID NO: 1110 - RNA, 202 nt, Glycine
aagagagugaaggagcuucccucagcccauucauggagauaacgaaagauuggguugcugaauuaacugcuagcucacacauucauucauacaauaguauucaauuaggguaauauugugugaaugaagcgggaguauauaguaucuauauugcaacccucuuucucugugcuuggacugaaaggagcuccuucuuuuucug

SEQ ID NO: 1111 - RNA, 193 nt, Glycine
auuaugaaguggagcuccuugaaguccaauugaggaucuuacugggugaauugagcugcuuagcuauggaucccacaguucuacccaucaauaagugcuuuugugguagucuuguggcuuccauaucuggggagcuucauuugccuuuauaguauuaaccuucuuuggauugaagggagcucuacacccuucu

SEQ ID NO: 1112 - RNA, 186 nt, Glycine
aaacccaacuuggaguucccugcacuccaagucugaaaggauaugaugguaaaccucuacugcuaguucauggauaccucugacuucuuaacaacaugcguucgaagucaaggguuugcaugcccugggagaugaguuuaccuugaucuuuugguauuggagugaagggagcuccagaggguauuc

SEQ ID NO: 1113 - RNA, 188 nt, Glycine
ccuaagguaagagagcuuucuucaguccacucaugggugacaguaagauucaauuagcugccgacucauucauccaaauguugaguguaagcgaauaaauauacucagcagaugagugaaugaugcgggagacaaauugaaucuuaaguuuccuguacuuggacugaagggagcucccuuuuccuuuu

SEQ ID NO: 1114 - RNA, 192 nt, Hordeum
guuuggagguggagcuccuaucauuccaaugaagggucuaccggaaggguuugugcagcugcuuguucaugguucccacuauccuaucuccauuagaacacgaggagauaggcuugugguuugcaugaucgaggagccgcuucgaucccucgcugaccgcuguuuggauugaagggagcucugcaucuugau

SEQ ID NO: 1115 - RNA, 189 nt, Liriodendron
guuauggacuaaggagcucucuucaguccaguccaagauaguauuaagccaaucuccgcugcugacucguuggcucaugaacucauccaacggcuaggauuccgauguguuuuugauccaacgaugcgggagccguguuugguucugucugucucgucuuggacugaagggagcucccuucuguuccac

SEQ ID NO: 1116 - RNA, 186 nt, Medicago
uuaaagggguggagcuuccuuuaguccaaauauggaucuugcuauguugauagagcugcuuagcuaugggucccucaacucuacccaucuuguucuuugugguaguuuuguggcuuccauaucuagggagccuuaucaccuuuaguuuaaucuuucuuuggauugaagggagcucuacaucuugcu

SEQ ID NO: 1117 - RNA, 270 nt, Oryza
uuguggacguugagcuccuuucgguccaaaaagggguguugcugugggucgauugagcugcugggucauggaucccguuagccuacuccauguucaucauucagcucgagaucugaaagaaacuacuccaauuuauacuaauaguauguguguagauaggaaaaugauggaguacucguuguugggauaggcuuauggcuugcaugccccaggagcugcaucaacccuacauggacccucuuuggauugaagggagcucugcaucuuuug

SEQ ID NO: 1118 - RNA, 267 nt, Oryza
uuguggacguugagcuccuuucgguccaaaaagggguguugcugugggucgauugagcugcugggucauggaucccguuagacuacuccauguucaucauucagcucgagaucugaaagaaacuacuccaauuuauacuaauaguauguguguagauaggaaaaugauggaguacucguugggauaggcuuauggcuugcaugccccaggagcugcaucaacccuacauggacccucuuuggauugaagggagcucugcaucuuugg

SEQ ID NO: 1119 - RNA, 244 nt, Pennisetum
gauuggaagcggagcuccuaucauuccaaugaaagguuguuuuuguggguugguacagcugcucguucaugguucccacuauccuaucucauggcuggagguuuaucugagagagagagagagagaugagaugaguggucggucugguguuggcuugagauaggcuuguggcuugcaugaccgaggagcugcaccguccccuugcuggccgcucuuuggauugaagggagcucugcaucuugau

SEQ ID NO: 1120 - RNA, 189 nt, Physcomitrella
accuugauuguggagcuccguuuucgguccaauaguggcugcgacggaagguggucccgcugccgaaucacacguccggguucuuuaucgggggcagggccccgauacgguauccgaacguuugucccgggaacuggucgaccuuccgcccggcgucucuuggacugaagggagcuccacucuuggcgu

SEQ ID NO: 1121 - RNA, 243 nt, Saccharum
uuugaagcggagcuccuaucauuccaaugaagggccguucugaaggguuguuccgcugcucguucaugguucccacuauccuaucucaucauguauguguguauguauuuucgagagggaggagaggagcuagacucucaugguggucgucuuugagauaggcuugugguuugcaugaccgaggagcugcaccguccccuugcuggccgcucuuuggauugaagggagcucugcauccugauc

SEQ ID NO: 1122 - RNA, 244 nt, Saccharum
gauuugaagcggagcuccuaucauuccaaugaagggccguucugaaggguuguuccgcugcucguucaugguucccacuauccuaucucaucauguauguguguauguauuuucgagagggaggagaggagcuagacucucaugggggucgucuaugagauacgcuugugguuugcaugaccgaugagcugcaccguccccuugcuggccgcucuuuggauugaagggagcucugcauccugau

SEQ ID NO: 1123 - RNA, 245 nt, Saccharum
gauuugaagcggagcuccuaucauuccaaugaagggccguucugaagggugguuccgcugcucguucaugguucccacuauccuaucucaucauguauguguguauguauuuucggagagggaggagaggagcuagacucucacgguggucgucuuugagauaggcuugugguuugcaugaccgaggagcugcaccguccccuugcuggccgcucuuuggauugaagggagcucugcauccugaa

SEQ ID NO: 1124 - RNA, 196 nt, Saccharum
ggaaagagagaggagcucccuucaauccaagcacgagggaaagaugaugguggguucaucucccgggucaugcacacccaugcaagugcaggugagcauuagucauugcugcaccagagaggcauccaugaaccggcagcugcaaccgaccacuuccccuccuggauuggaucgaagggagcucuucgaucacuuu

SEQ ID NO: 1125 - RNA, 194 nt, Saccharum
aagugaucgaagagcucccuucgauccaauccaggaggggaaguggucgguugcagcugccgguucauggaugccucucuggugcagcaaugacuaaugcucaccugcacuugcaugggugugcaugacccgggagaugaacccaccaucaucuuucccucgugcuuggauugaagggagcuccucucucuuuc

SEQ ID NO: 1126 - RNA, 244 nt, Saccharum
gauuugaagcggagcuccuaucauuccaaugaagggccguucugaagggugguuccgcugcucguucaugguucccacuauccuaucucaucauguauguguguauguauuuucgagagggaggagaggagcuagacucucacgguggucgucuuugagauaggcuugugguuugcaugaccgaagagcugcaccguccccuugcuggccgcucuuuggauugaaaggagcucuugcaucugau

SEQ ID NO: 1127 - RNA, 193 nt, Schedonorus
guuuugagguggagcuccuaucauuccaaugaaaggucuugcuagaaggggugguacagcugcucguucaugguucccacuauccuaccuccguuugaaaccagggagauaggccuguggcuugcaugaccgaggagccgcaucguccccucgcuggccgcucuuuggauugaagggagcucugcaucuaggc

SEQ ID NO: 1128 - RNA, 240 nt, Sorghum
gauucgaagcggagcuccuaucauuccaaugaagggcccuuuucaugggugguuccgcugcucguucaugguucccacuauccuaucucaucauguaucuguguauguacucuagagggccggagaagagauucauguggucgucagucuuugagauaggcuugugguuugcaugaccgaggagcugcaccguccccuugcuggccgcucuuuggauugaagggagcucugcauccugau

SEQ ID NO: 1129 - RNA, 240 nt, Sorghum
gauucgaagcggagcuccuaucauuccaaugaagggcccuuuucaugggugguuccgcugcucguucaugguucccacuauccuaucucaucauguaucuguguauguacucuagagggccggagaagagauucauguggucgucagucuuugagauaggcuugugauuugcaugaccgaggagcugcaccguccccuugcuggccgcucuuuggauugaagggagcucugcauccugau

SEQ ID NO: 1130 - RNA, 240 nt, Sorghum
gauucgaagcggagcuccuaucauuccaaugaagggcccuuuucaugggugguuccgcugcucguucaugguucccacuauccuaucucaucauguauguguguauguacucuagagggcccgagaagagauucauguggucgucagucuuugagauaggcuugugguuugcaugaccgaggagcugcaccguccccuugcuggccgcucuuuggauugaagggagcucugcauccugau

SEQ ID NO: 1131 - RNA, 213 nt, Triticum
aguuugagggagcucacuucaguccacucaugggagguagcggggauugaacgagcugccgacucauucacucgagcacacaguagauaugagacuaguccagggcauaccaguauguuacaauauguacugugcgaaugagcgaaugcagcgggagauuguucucucuuuccuccuccaugcuuggacugaagggagcucccucaucucuca

SEQ ID NO: 1132 - RNA, 270 nt, Triticum
uuguggacguugagcuccuuucgguccaaaaagggguguugcugugggucgauugagcugcugggucauggaucccguuagccuacuccauguucaucauucagcucgagaucugaaagaaacuacuccaauuuauacuaauaguauguguguagauaggaaaaugauggaguacucguuguugggauaggcuuauggcuugcaugccccaggagcugcaucaacccuacauggacccucuuuggauugaagggagcucugcaucuuugg

SEQ ID NO: 1133 - RNA, 191 nt, Triticum
guuuagagguggagcuccuaucauuccaaugaagggucuaccggaaggguuugugcagcugcucguucaugguucccacuauccuaucuccauagaaaacgaggagagaggccugugguuugcaugaccgaggagccgcuucgaucccucgcugaccgcuguuuggauugaagggagcucugcaucuugau

SEQ ID NO: 1134 - RNA, 190 nt, Vitis
guuuuggaguggagcuccuugaaguccaauagagggucuuacuggguagauugagcugcugacuuauggaucccacagcccuaucccgucaauggggggcauuggauaggcuuguggcuugcauaucucaggagcugcauuauccaagcuuagauccuuguuuggauugaagggagcucuacaccucucu

SEQ ID NO: 1135 - RNA, 107 nt, Vitis
cugcagaaauggggguuccuuugcagcccaaaacaacuccaucgcugaagaagaugaugaacuucaugcuccuuguuuuggacugaagggagcuccuaguucuucuc

SEQ ID NO: 1136 - RNA, 186 nt, Vitis
gguuuggagugagcuccuugaguccaauagaggucuuacuggguagaugagcugcugacuuauggaucccacagcccuaucccgucaauggggggcauuggauaggcuuguggcuugcauaucucaggagcugcauuauccaaguuuagauccuuguuuggauugaagggagcucuacaccucucu

SEQ ID NO: 1137 - RNA, 196 nt, Zea
aggcgaucgaagagcucccuucgauccaauccaggaggggaaguggucgguugcagcugccgguucauggauaccucucuggugcagcaauggccgcugcucaccucugcacuugcaugggugugcaugacccgggagaugagcccgccaucaucuuucccucgugcuuggauugaagggagcuccucucugucug

SEQ ID NO: 1138 - RNA, 198 nt, Zea
acagacagagaggagcucccuucaauccaagcacgagggaaagaugauggcgggcucaucucccgggucaugcacacccaugcaagugcagaggugagcagcggccauugcugcaccagagagguauccaugaaccggcagcugcaaccgaccacuuccccuccuggauuggaucgaagggagcucuucgaucgccuu

SEQ ID NO: 1139 - RNA, 98 nt, Glycine
auguguaugugccuggcucccuguaugccauuuguagagcucaucgaagcaucaaugaccuuuguggauggcguaugaggagccaagcauauuucaua

SEQ ID NO: 1140 - RNA, 150 nt, Oryza; identical to SEQ ID NO: 799

SEQ ID NO: 1141 - RNA, 102 nt, Oryza; identical to SEQ ID NO: 797

SEQ ID NO: 1142 - RNA, 107 nt, Triticum
ugauaugaugugccuggcucccuguaugccacucauccagagcaacaccuuuugcaauaagguugccugcgauggauggcgugcacggauccaagcauaucgaaccc

SEQ ID NO: 1143 - RNA, 101 nt, Zea
gugucgugugugccuggcucccuguaugccacacauguagccaacccguggcgugauuggaugcuguggguggcgugcaaggagccaagcaugcauaacag

SEQ ID NO: 1144 - RNA, 98 nt, Lupinus
gugaagucacuggaagcagcgguuuaucgaucucuuccugaauuugauuaacacaaaccaugaaucgaucgauaaaccucugcauccagcgcucacuu

SEQ ID NO: 1145 - RNA, 107 nt, Medicago
aaguucgucacuggaugcagcgguucaucgaucuguuccugaauuuuguuugucucguaaaacaaacaugaaucggucgauaaaccucugcauccagcgcucacuuu

SEQ ID NO: 1146 - RNA, 132 nt, Oryza
gggugaugccugggcgcagugguuuaucgaucucuucccugccuugugcugcuccgaucgaugcccgugcugauucuugauaauauacaacgcaggaaucgaucgauaagccucugcauccagaucucacuu

SEQ ID NO: 1147 - RNA, 103 nt, Vitis
uugaagucacuggaugcagcgguucaucgaucucuuccugaaauuguugugaaaaaagcagaucaagaaucggucgauaaaccucugcauccagcguucacuc

SEQ ID NO: 1148 - RNA, 155 nt, Populus
ugagcaagauggagaagcagggcacgugcauuacuaacucaugcacacagagugagagagacauuucuugcuggaguuaugacucuuaccuacaauagauuuuguuggcuucagcgaguuaguucuucaugugccugucuuccccaucaugauca

SEQ ID NO: 1149 - RNA, 155 nt, Populus
ugagcaagauggagaagcagggcacgugcauuacuaacucaugcauacagagugagagagacauuucuugcucgaguuaugacucuuaccuacuauagauuuuguuggccucagugaguuaguucuucaugugccugucuuccccaucaugauca

SEQ ID NO: 1150 - RNA, 170 nt, Triticum
cgcgcgagguggagaagcagggcacgugcauccauuuccagcucggcauucccggcguccggccggccggcugccgcggccuugccuggcuggguagugcgucgcucgauccggccgugcgccggcggccggcccuugcaugcaugugccuuucuucuccaccgugcaca

SEQ ID NO: 1151 - RNA, 156 nt, Glycine
cacgucuugaggggaaugcagugugguccaaggagaugauauaucacuucaccauacucauaucuucacaaacucaucagauucagauguaauaauaauuuguaauauaugcauauuaugucuccucggaccaggcuucauucccuucaauuacag

SEQ ID NO: 1152 - RNA, 127 nt, Glycine
ugucuuuugaggggaauguugucuggcucgaggacccuucuucaucuugaucuuguguagacuacuaugcuuguggucaaggaauacauaguguugucggaccaggcuucauuccccccaauuauau

SEQ ID NO: 1153 - RNA, 174 nt, Glycine
ugggguugaugggaauguuguuuggcucgagguaacuaugcauggucuuaauuuuguucaucuuugaagcuuuaauuuauggguuucgaucucuuugaucccuugaaacaaagaaagcuuuaaagguuggauuuugaggcuuucucggaccaggcuucauucccguaaaccuua

SEQ ID NO: 1154 - RNA, 103 nt, Glycine
agguguugaggggaauguuggcuggcucgaggcuuuucaaagaggagguucucacuggcaagaacuauaaggcuucggaccaggcuucauuccccucaaaaau

SEQ ID NO: 1155 - RNA, 146 nt, Glycine
ugagguugagaggaauguugucuggcucgaggucauggaggaggaggaggaguagaguacugagaucagugaaaguuuccaauggaaauuuacccucuuacacaaaaaaaugauucucggaccaggcuucauuccccccacccaac

SEQ ID NO: 1156 - RNA, 185 nt, Hedyotis
uaucuuuugaggggaauguugucuggugcgaggccaccaacuagauccauggaauccuucuuuauauauuauacagaucuuuucuuuugaaggguuuugaccauuuugauuuuguuugauuuaagguuaagagguggaucuugcguuagugucgucggaccaggcuucaucccccccaauuguuu

SEQ ID NO: 1157 - RNA, 100 nt, Hordeum
  misc_feature (22)..(22): n is a, c, g, or u
augguugucgaggggaaugacnccggguccnaaagagagacncucgcauggcgugcgcguggugcguuucggaccaggcuucauuccccaugacuccauc

SEQ ID NO: 1158 - RNA, 188 nt, Ipomoea
ggaguuugagggggauguuggcuggcucgaugcacuuacuuaucaucuuccucaaaaacaugcgacauauacauacauauggaagaucauauaucuauaaauauauauuugucuuccauaucccauauauauauguugaugaugguggagugauggcaucggaccaggcuucauuccucccaaaacac

SEQ ID NO: 1159 - RNA, 114 nt, Medicago
guuagguugagaggaacguugucuggcucgaggugauggagauggaagaguacucucuacucacucaucacuaacuuucaaucucggaccaggcuucauuccccccagcaaacu

SEQ ID NO: 1160 - RNA, 114 nt, Medicago
cuuauuugaggggaauguuggcuggcucgaggcuuuucaguuucacaaaggaaguucagucuuaauuguaugaacuauaaggcuucggaccaggcuucauuccccucaaaaucu

SEQ ID NO: 1161 - RNA, 117 nt, Medicago
uauuuuuugaggggaauguugucuggcucgaggacgcuuucuucucgaucuaaugcaaauuuguggucauggauuguaaaguauucucggaccaggcuucauuccccccaauuauau

SEQ ID NO: 1162 - RNA, 182 nt, Medicago
aggagguguuuggaaugagguuuguuccaagaucaucacacaucauguuucuucuucccuuucaauaauucuugauuaauuuauguucauacaucuauauauauaucuauauauauuaucaagucauuacaugcaugggaugagauauugagagugaucucggaccaggcuucauuccucac

SEQ ID NO: 1163 - RNA, 115 nt, Oryza
uuagguuaaggggauuguugucugguucaaggucuccacauugugcaaaauguucauucauggaggcacaggaugcuuggugaucucggaccaggcuucaaucccuuuaaccagg

SEQ ID NO: 1164 - RNA, 128 nt, Sorghum
uugcuucugaguggaauguugucugguucaaggucucgcuugugauuuaaggaugauuugugcaugcguaauuuuuauuccuugaaucuaugagaucucggaccaggcuucauuccccucagcaauag

SEQ ID NO: 1165 - RNA, 107 nt, Zea
cuuacuuugaggggaauguugucuggcucgaggugcagaaacaugcagaucucaucggucuagguucuugucgaucucggaccaggcuucauuccccucaaguggag

SEQ ID NO: 1166 - RNA, 82 nt, Glycine
ugcagcaguugaagcugccagcaugaucugaguuuaccuucuauugguaagaacagaucauguggcugcuucaccuguugaa

SEQ ID NO: 1167 - RNA, 102 nt, Glycine
aaggaaaaagugaagcugccagcaugaucuagcuuugguuagugggagcgagauagugcuaacccucacuaggucaugcugugcuagccucacuccuuccua

SEQ ID NO: 1168 - RNA, 110 nt, Oryza; identical to SEQ ID NO: 824

SEQ ID NO: 1169 - RNA, 118 nt, Oryza
acaaguuggugaagcugccagcaugaucugaugaugaugaugauccaccucucucaucuguguucuugauuaauuacggaucaaucgaucaggucaugcuguaguuucaucugcuggu

SEQ ID NO: 1170 - RNA, 87 nt, Saccharum
acuaccaguugaagcugccagcaugaucuuaacuucccucacuugguugaggagagaucagaucauguggcaguuucaccuaguugu

SEQ ID NO: 1171 - RNA, 167 nt, Saccharum
acaaguuggugaagcugccagcaugaucugauggugguauauaugaauauaugaugucuuuaccucugaucucucccugacugucacggauccaugaauccaggaugaggggagggaagaaagagggauaaugagcaucaggucaugcuguaguuucaucugcuggu

SEQ ID NO: 1172 - RNA, 169 nt, Saccharum
cacaaguuggugaagcugccagcaugaucugauggugguauauaugaauauaugaugucuuuaccucugaucucucccugacugucaccgauccaugaauccaggaugaggggagggaagaaagagggauaaugagcaucaggucaugcuguaguuucaucugcuggug

SEQ ID NO: 1173 - RNA, 157 nt, Saccharum
acaaguuggugaagcugccagcaugaucugauggugguauauaugaauauaugaugucuuuaccucugaucucucccugacugucacggaucgaugaauccaggaugaggggagggaauaaugagcaucaggucaugcuguaguuucaucugcuggu

SEQ ID NO: 1174 - RNA, 100 nt, Zea
cucuaguagcugaagcugccagcaugaucugagguguccacagcauauauauggaagcagcuagcgaucagaucaugcugugcaguuucaucugcucgug

SEQ ID NO: 1175 - RNA, 122 nt, Arabidopsis
ggcucggauucgcuuggugcaggucgggaaccaauucggcugacacagccucgugacuuuuaaaccuuuauugguuugugagcagggauuggaucccgccuugcaucaacugaaucggaucc

SEQ ID NO: 1176 - RNA, 184 nt, Betula
gucucuaauucgcuuggugcaggucgggaacugcuucgcuuuugcccuuagaacacgcauacauguuugagaguacuguuaaagucuuuagccagcuggcgcguugcgguuggcuggcuggcuguggucguguggggaauauagggcgaauuggaucccgccuugcaucaacugaaucggagac

SEQ ID NO: 1177 - RNA, 106 nt, Glycine
gucucuaauucgcuuggugcaggucgggaaccgguuuucgcgcggaauggaggagcggucgccggcgccgaauuggaucccgccuugcaucaacugaaucggaggc

SEQ ID NO: 1178 - RNA, 158 nt, Hedyotis
gucuccgauucgcuuggugcaggucgggaacugcuucuucacucacggaaagaaaaauaugugccuuuacagagaagcaacguuuuuuauuuuauaauuuuuugaacgacguuucuggugaauuuaaguucaugccuugcaucaacugaauuggauga

SEQ ID NO: 1179 - RNA, 163 nt, Lycopersicon
gccucuuauucgcuuggugcaggucgggaccucauucgccggcgccgggaauaaugccggacgaacgacggcgguguuaauucuacuaaagcugucaccgacggauagauguuugauuagcggcgaaauuuggguccugccuugcaucaacugaauuggagac

SEQ ID NO: 1180 - RNA, 163 nt, Lycopersicon
gccucuuauucgcuuggugcaggucgggaccucauucggcggcgccgggaauaaugccggacgaacgacggcgguguuaauucuacuaaagcugucaccgacggauagauguuugauuagcggcgaaauuuggguccugccuugcaucaacugaauuggagac

SEQ ID NO: 1181 - RNA, 85 nt, Oryza
gccucgggcucgcuuggugcagaucgggacccgccgccgccgcugccggggccggaucccgccuugcaccaagugaaucggagcc

SEQ ID NO: 1182 - RNA, 136 nt, Populus
gucucuaauucgcuuggugcaggucgggaacugauucugcgauuucauugccagauggcuaaacacgauuggcugugaggcaaauuauaaaaagaaagagaauuggaucccgccuugcaucaacugaaucggagac

SEQ ID NO: 1183 - RNA, 158 nt, Populus
gucucugauucgcuuggugcaggucgggaacugauucggcgauuugauugccagcuggcuggacaugacugguuguuauggaaaaagaaaaggaaggaaacaggaaaaaacaaagaauagcgaauuggaucccgccuugcaucaacugaaucggaggc

SEQ ID NO: 1184 - RNA, 83 nt, Saccharum
gccucgggcucgcuuggugcagaucgggacccgccgcccggccgacgggacggaucccgccuugcaccaagugaaucggagcc

SEQ ID NO: 1185 - RNA, 83 nt, Saccharum
gccucaggcucgcuuggugcagaucgggacccgccgcccggccgacgggacggaucccgccuugcaccaagugaaucggagcc

SEQ ID NO: 1186 - RNA, 151 nt, Solanum
gccucucauucgcuuggugcaggucgggaccuaccucgccggcacaauggcgguagcugacggcgacgccagcguaccgguaaaaacuaauuuuuuacauguugucuguggcguaguuugggucccgccuugcaucaacugaauaggagac

SEQ ID NO: 1187 - RNA, 152 nt, Solanum
gccucucauucgcuuggugcaggucgggaccuaccucgccggcaacaauggcgguagcugacggcgacgccagcguaccgguaaaaacuaauuuuuuacauguugucuguggcguaguuugggucccgccuugcaucaacugaauaggagac

SEQ ID NO: 1188 - RNA, 151 nt, Solanum
gccucucauucgcuuggugcaggucgggaccuaccucgccggcaacaauggcgguagcugacggcgacggcagcuuaccgguaaaaacuuuuuuuuuuuacaugucuguggcguaguuugggucccgccuugcaucaacugaauaggagac

SEQ ID NO: 1189 - RNA, 85 nt, Sorghum
gccucgggcucgcuuggugcagaucgggaccugccgccgugcucggacgggacagaucccgccuugcaccaagugaauccgagcc

SEQ ID NO: 1190 - RNA, 128 nt, Vitis
gucucuaauucgcuuggugcaggucgggaaccgacuucgccgcuccggcagcgccggaggcacgcggcggccuacgauugguugcugagcgaauuccgaucccgccuugcaucaacugaaucggagac

SEQ ID NO: 1191 - RNA, 83 nt, Zea
gucucgggcucgcuuggugcagaucgggacccgccgcccggccgacgggacggaucccgccuugcaucaagugaaucggagcc

SEQ ID NO: 1192 - RNA, 83 nt, Zea; identical to SEQ ID NO: 1184

SEQ ID NO: 1193 - RNA, 83 nt, Zea
gccucgggcucgcuuggugcagaucgggacccgccgcccggccgacgggacggaucccgccuugcaccaagcgaaucggagcc

SEQ ID NO: 1194 - RNA, 129 nt, Glycine
aguguagugcagccaaggaugacuugccggcauuagccaagugaaugagcaucauauauauauauauauauauauauauauauaugacucauguucuugucggcaaguuggccuuggcuauauuggacu

SEQ ID NO: 1195 - RNA, 121 nt, Glycine
aguguagugcagccaaggaugacuugccggcauuagccaagugaaugagcaucauauauauauauauauauauauaugacucauguucuugucggcaaguuggccuuggcuauauuggacu

SEQ ID NO: 1196 - RNA, 100 nt, Glycine
agagugaugcagccaaggaugacuugccggcguuauuauuugcucauguucaugcucaccgguuuccuugccggcaaguuguguuuggcuauguuuugcu

SEQ ID NO: 1197 - RNA, 113 nt, Oryza
gccauggugcagccaaggaugacuugccgaucgaucgaucuaucuaugaagcuaagcuagcuggccauggauccauccaucaauuggcaaguuguucuuggcuacaucuuggc

SEQ ID NO: 1198 - RNA, 110 nt, Oryza
aacgggaugcagccaaggaugacuugccggcuccugguauugggggaaucucagcuuugcugaagcgccuuggaguuagccggcaagucuguccuuggcuacaccuagcu

SEQ ID NO: 1199 - RNA, 108 nt, Oryza
cgacucagacuagccaaggagacugccuaugaaccaguucaaggcucauuuucugaaugauccucuacacaaggacacaggcaagucauccuuggcuaccagagcuac

SEQ ID NO: 1200 - RNA, 109 nt, Oryza
gccauggugcagccaaggaugacuugccgaucgaucuaucuaugaagcuaagcuagcuggccauggauccauccaucaauuggcaaguuguucuuggcuacaucuuggc

SEQ ID NO: 1201 - RNA, 108 nt, Oryza; identical to SEQ ID NO: 848

SEQ ID NO: 1202 - RNA, 125 nt, Oryza
ccacucaggcuagccaaggagacugccuaucucggcucaucugaaccagccaggccacacugaucauggcacugcaucauucagaagagcacaauaggcaagucauccuuggcuaccagaggcag

SEQ ID NO: 1203 - RNA, 127 nt, Oryza
uugccucuguuagccaagaauggcuugccuaucuccacuauuugguucaucacuggaacccacuugggguucuccgaugguggaugaaauauggaagauggugagccuucauggcuaagagagugau

SEQ ID NO: 1204 - RNA, 119 nt, Populus
agauugaugcagccaaggaugacuugccgacgacucgcuuuugcuuuugcuuccaucaauauaggcauaaucaagaagagaugaauccguuggcagguuguucuuggcuacauuuuucu

SEQ ID NO: 1205 - RNA, 102 nt, Populus
aguuugaugcagccaaggaugauuugccgacgacucaguuuuugcuuccauaugguaggagagaugaagagguuggcagguuuuccuuggcuacauuuuccu

SEQ ID NO: 1206 - RNA, 119 nt, Triticum
ccaucuucgauagccaaggaugauuugccugugaaacucccuuggcagccgagcucucugccacagagagcggcguccggcgguuccaugggcaagucacccugggcuacccgaaguac

SEQ ID NO: 1207 - RNA, 113 nt, Zea
gaacuagguguagccaaggacagacuugccgacugaguucuaagccucagcagcaagcugagacgccuccagguuccaggagccggcaagucauccuuggcugcaucccguuc

SEQ ID NO: 1208 - RNA, 112 nt, Arabidopsis; identical to SEQ ID NO: 511

SEQ ID NO: 1209 - RNA, 97 nt, Glycine
guucaacgggauauugguccgguucaauaagaaagcaaugcucaaaauguuuuuggguccuguuuuuucauugagccgugccaauaucacgaaccac

SEQ ID NO: 1210 - RNA, 97 nt, Hedyotis
cauaaacgagaucuuggugcgguucaaugguaacgguuguggucguaacauuaagaccccaauuuuucgauugagccgugccaauaucacguacuau

SEQ ID NO: 1211 - RNA, 96 nt, Hordeum
ggucacuaugauguuggcucgacucacucagaccacgccggagggagccaucugcggcggcgguucugauugagccgugccaauaucuuagugcuc

SEQ ID NO: 1212 - RNA, 127 nt, Oryza
gggagagugcgauguuggcaugguucaaucaaaccgggcaaacuuaugcacuagcuaagcaagaugcagggaucugcaguaugguuuuguuuggucugauugagccgugccaauaucacaagcuugc

SEQ ID NO: 1213 - RNA, 117 nt, Oryza
gguagcuaugauguuggcucggcucacucagacggcauuggcgugaugcaaagcaugcaugcgugcuugcuagcucacuuguguuucugauugagccgugccaauaucuuagugcuc

SEQ ID NO: 1214 - RNA, 98 nt, Triticum
agugaacgcgguauuggugcgguucaaucagagagcuggcgccccaggaggcaagggguuccucccuucgauugagccgugccaauaucacgcgguuu

SEQ ID NO: 1215 - RNA, 114 nt, Triticum
uggucacuaugauguuggcucgacucacucagaccacgccugccggccggccguagccaugcaucugcaugcggugguggcucugauugagccgugccaauaucucagugcucu

SEQ ID NO: 1216 - RNA, 133 nt, Zea
gagagagugcgauguuggcauggcucaaucaacucgccggccgcggguggcuuagcuuauuaauucugcguuuuugaucgaggugcgggcgcaguguuuaauugauugagccgugccaauaucacaaccuucu

SEQ ID NO: 1217 - RNA, 131 nt, Zea
agagagugcgauguuggcauggcucaaucaacucgccggccgcggguggcuuagcuuauuaauucugcgcguucgaucgaggugcgggcgcaguguuuaauugauugagccgugccaauaucacaaccuuc

SEQ ID NO: 1218 - RNA, 125 nt, Citrus
ugcucgcuguagcagcguccucaagauucacauccagucuaaaggcaaaagcagcaauuuuucuucauuuuugcuugccuugguuuuugucagugagaaucuugaugaugcugcaacggcgauua

SEQ ID NO: 1219 - RNA, 132 nt, Glycine
uguuugcggauguagcaucaucaagauucacaugcaaaugaaggugggugggacuaugaugcaauccaagugcucugccaauccaucggucuuuuugaugugagaaucuugaugaugcugcaucagccauaa

SEQ ID NO: 1220 - RNA, 140 nt, Glycine
uuauuugcggauguagcaucaucaagauucacaugcaagcgcagguggugggugggacuugaugcaaucuaagugcugugccagccaagccauaggucuuuuggaacugagaaucuugaugaugcugcaucagccauaaa

SEQ ID NO: 1221 - RNA, 124 nt, Lycopersicon
uguuugcauauguggcauaaucaagauucacgugaaaaguugcaaauugguuauauaauugaugaaauuaauggcuggcuauuugaaacucacgagaaucuugaugaugcugcaucagcaauaa

SEQ ID NO: 1222 - RNA, 111 nt, Solanum
ugcuugcuagugcagcaccaucaagauucacauagaaaauauggacuaugaaaugaaauaugcccaauuuuugaauacaugagaaucuugaugaugcugcauuggcaaauu

SEQ ID NO: 1223 - RNA, 113 nt, Oryza
ggggaagcauccaaagggaucgcauugauccuucaucgcucucgcucgcuuccauggcggucgucgucuacaagcagcuugacggaucaugcgauccuuuuggaggcuuccuc

SEQ ID NO: 1224 - RNA, 113 nt, Oryza
guggaggacuccaaagggaucgcauugaucuggcuagcuaucucgaucgaucgccucaucgaucgacgacgacgugcgugaucgaucagugcaaucccuuuggaauuuuccuc

SEQ ID NO: 1225 - RNA, 107 nt, Populus
uuggaguguuccaaagggaucgcauugaucuaaugacuuucgaugucuauaugauguuaauguuuagucauuucauuggaucaugcgaucccuuaggaauuuuccag

SEQ ID NO: 1226 - RNA, 83 nt, Glycine
cagaguuucuuggcauucuguccaccuccacuucuuggcccuaucuacguacucggagguggauauacugccaauagagcugu

SEQ ID NO: 1227 - RNA, 97 nt, Glycine
cagaguuuauuggcauucuguccaccuccacuuccuacucucucucugagccacauguucgugaaguuggaggugggcauacugucaacugaguucu

SEQ ID NO: 1228 - RNA, 106 nt, Robinia
cagaguuucuuggcauucuguccaccuccacuuucauccuccuguuucuguuaauggaucucucucaccuaauauguggaggugggcauacugccaacagagcugu

SEQ ID NO: 1229 - RNA, 89 nt, Glycine
guugucucuuggaguuccucugaacgcuucaugugauuggcuaguuauaggccuuugaugaaguguuugggggaacucuuagguucaac

SEQ ID NO: 1230 - RNA, 86 nt, Oryza
uauuaucgugaguucccuucaagcacuucacauggcccuauuucaaugucuaauaugugaaguguuugggggaacucuugguaucg

SEQ ID NO: 1231 - RNA, 86 nt, Oryza
cauugucgugaguucccuucaagcacuucacguggcacuauuucaaugcguaccgugugaaguguuugggggaacucuuggcaucc

SEQ ID NO: 1232 - RNA, 87 nt, Oryza
uauuacuaugaguucucuuuaagcacuucauacgacaccauuauuguuaggguuguuaugaaguguuuggaggaacucucagugcca

SEQ ID NO: 1233 - RNA, 142 nt, Oryza
uuguccacuggaguucuccucaauccacuucaguagauagcuauggcuaggccucauugcauugcacuguuacauaacugugaucauggggccaaaagcuagcuauguacagugaagugcuugggggaacuccaguugacac

SEQ ID NO: 1234 - RNA, 84 nt, Oryza
agucccuaggaguuccuuucaagcacuuuacgacacaccguauugagaguugucgugaaguguuugggggaacucuuagugucg

SEQ ID NO: 1235 - RNA, 87 nt, Oryza
uauuaucaagaguucucuuuaagcacuucauacgacaccauuauuuauaggguuguugugaaguguuuggaggaacucucaguguca

SEQ ID NO: 1236 - RNA, 86 nt, Oryza
uauugucgugaguuccauucaagcacuucacguggcacuaucucaaugccuacuaugugaaguguuugggggaacucucgguauca

SEQ ID NO: 1237 - RNA, 82 nt, Triticum
gucuuagcauggguuccuuacaagcacuucacuaggcauugaaaugccaaugugaaguguuuggagggacucuuaguggcau

SEQ ID NO: 1238 - RNA, 84 nt, Triticum
ucuuaccauggguuccuugcaagcacuucaugaggcauuauuugagaugccacuaugaaguguuugggggaacucuuggugaug

SEQ ID NO: 1239 - RNA, 137 nt, Brassica
gguuauauuuuuccacagcuuucuugaacuuucuuuuucauuucccuuauuuuasagcgaaauuaaauaacuaaaaaucucuaacauuuaacacucuasaaaaaaaaagcucaasaaagcugugggaaaacaugaca

SEQ ID NO: 1240 - RNA, 105 nt, Glycine
gucaugcuuuuccacagcuuucuugaacuucuuaugcaucuuauaucucuccaccuccaggauuuuaagcccuagaagcucaagaaagcugugggagaauauggc

SEQ ID NO: 1241 - RNA, 119 nt, Glycine
uuuguauucuuccacagcuuucuugaacugcauccaaagaguuccuuugcaugcaugccauggcacucuuacucccaaaucuuguuuugcgguucaauaaagcugugggaagauacaga

SEQ ID NO: 1242 - RNA, 199 nt, Mesembryanthemum
uuuguauucuuccacagcuuucuugaacugcaucuucccuaauuuuuuuaucuuuuguuuuuccucucucagauccaauuuuuucuuugguaaauacuuauaauuuaucaaaaaaaaaaaagauggauuuuugggaauugaaagauggaaaauuuuaauuuguggguugcgguucaauaaagcugugggaagauacaaa

SEQ ID NO: 1243 - RNA, 200 nt, Mesembryanthemum
uuuguauucuuccacagcuuucuugaacugcaucuucccuaauuuuuuuaucuuuuguuuuuccucucucagauccaauuuuuucuuugguaaauacuuauaauuuaucaaaaaaaaaaaaagauggauuuuugggaauugaaagauggaaaauuuuaauuuguggguugcgguucaauaaagcugugggaagauacaaa

SEQ ID NO: 1244 - RNA, 201 nt, Mesembryanthemum
uuuguauucuuccucagcuuucuugaacugcaucuucccuaauuuuuuuaucuuuuguuuuuccucucucagauccaauuuuuucuuugguaaauacuuauaauuuaucaaaaaaaaaaaaagauggauuuuugggaauugaaagauggaaaauuuuaauuuguggguugcgguucaauaaagcugugggaagauacaaau

SEQ ID NO: 1245 - RNA, 118 nt, Mesembryanthemum
cgccauauuuucccacagcuuucuugaacuuucccaaugaugguuuguuucucacuagaaagaaaaaaaaagaagaaaagaaccggaaaguucaagaaagcuguggaaaagcauggca

SEQ ID NO: 1246 - RNA, 152 nt, Oryza
uuugugaucuuccacagcuuucuugaacugcacgcaugaugaauaaucccuuugguuaauugugaucuggucucugagagaucguagcuagacucgaucgguugcauuggcaucagagagagcaguucaauaaagcugugggaaauugcaga

SEQ ID NO: 1247 - RNA, 132 nt, Populus
uuuguauucuuccacagcuuucuugaacugcaccuauuagauuuauguugauguuguugugcgauuugccaugaccauaugacauuguauucauuuuugcugcgguucaauaaagcugugggaagauacaaa

SEQ ID NO: 1248 - RNA, 132 nt, Populus
uuuguauucuuccacagcuuucuugaacugcaccuauuagauuuauguugauguuguugugcgauaugccaugaccauaugacauuguauucauuuuugcugcgguucaauaaagcugugggaagauacaaa

SEQ ID NO: 1249 - RNA, 131 nt, Prunus
uuuguauucuuccacagcuuucuugaacugcauccaugagaucgaucgaucuuugcaugugaggcugcagucacucacucacucucucucuaacuggcuugcgguucaauaaagcugugggaagauacaga

SEQ ID NO: 1250 - RNA, 131 nt, Prunus
uuuguauucuuccacagcuuucuugaacugcauccaugagaucgaucgaucuuugcaugugaugcugcagucacucacucacucucucucuaacuggcuugcgggucaauaaagcugugggaagauacaga

SEQ ID NO: 1251 - RNA, 113 nt, Saccharum
uuugugaucuuccacagcuuucuugaacugcaucucuaagaggagcagcucgaagccucgaacucuaccugcaugagcaggugcaguucaauaaagcugugggaaacugcaga

SEQ ID NO: 1252 - RNA, 121 nt, Solanum
gucaugcuuuuccacagcuuucuugaacuucuucuugcuaaauuuugaucucuaaauugauaauuuugagaugaaauuuuugaagcuaugaaaguccaagaaagcugugggaaaagauggc

SEQ ID NO: 1253 - RNA, 117 nt, Zea
cuuugugaucuuccacagcuuucuugaacugcaucuuucagaggagcggcaguuucaacuccuccacccgcaucagcaggugcaugcaguucaauaaagcugugggaaacuggaaag

SEQ ID NO: 1254 - RNA, 128 nt, Zea
ccugccaucuuccacagcuuucuugaacugcaucaugcaugcagcaggcugugcuguggaccugaucgaguuucaauugauccaagcaagcaagagggcaguucaauaaagcugugggaaauugcaga

SEQ ID NO: 1255 - RNA, 107 nt, Hordeum
gcagaggugccguugagugcagcguugaugaaccguccggccauggcccguccgccuccaccgaggccggagcgguucaccggcgcugcacgcaaugacgccucugc

SEQ ID NO: 1256 - RNA, 107 nt, Hordeum
  misc_feature (47)..(47): n is a, c, g, or u
gcagaggugccguugagugcagcguugaugaaccguccggccauggnccguccgccuccaccgaggccggagcgguucaccggcgcugcacgcaaugacgccucugc

SEQ ID NO: 1257 - RNA, 153 nt, Citrus
gugaaccccagaggagugaaccugagaacagaggguggcguuggcuuaaauuugauuugcauugcugcugccugcuccugccauacauuaaaucaaaaagauuguauguggccaacgcaccuuguguucucaggucaccccuuugggaauuca

SEQ ID NO: 1258 - RNA, 118 nt, Glycine
aggaauucuacagggucguccugagaccacaugaaacagauucaaaauacaagcauauuugcuugugaccuuuugugacucaguucauguguucucaggucgccccugcugaacuuuu

SEQ ID NO: 1259 - RNA, 116 nt, Glycine
guuuaucucagaggaguggaucugagaacacaaggcugguuugcacugcuauauuaugaucgauugguauaaggugaauuuacuuuguguucucaggucaccccuuugagccaacc

SEQ ID NO: 1260 - RNA, 107 nt, Glycine
cucggaggagugaaucugagaacacaaggcugguuugcacugcuauaucaucuauugguauaaggugaauuuacuuuguguucucaggucaccccuuugagccaacc

SEQ ID NO: 1261 - RNA, 84 nt, Helianthus
gacauccaacaggugugacaugagaacacaugaugauauacuuagauauuuucacguguucucaggucgccccugcugaauuuu

SEQ ID NO: 1262 - RNA, 93 nt, Lactuca
aggcauccaacaggugcgacaugggaacacauguuaaaugugcaacaaaucacauuccgccauguguucucaggucgccccugcaggguuuuu

SEQ ID NO: 1263 - RNA, 93 nt, Lactuca
aggcauccaacaggugcgacauggcaacacauguuaaaugugcaacaaaucacauuccgccauguguucucaggucgccccugcaggguuuuu

SEQ ID NO: 1264 - RNA, 133 nt, Lotus
gucuaucucaaaggagugagccugagaacacaagcugaauugguuugaauugccauaucacauacugauaucugguauaggcuuuauguugcuaauuuauuuuguguucucaggucaccccuuugagcugacc

SEQ ID NO: 1265 - RNA, 108 nt, Medicago
uucuaucucagaggagugacacugagaacacaagauugauuaaucauauaauguauuugguuguuacuaguugauuuuguguucucaggucaccccuuugagucaacc

SEQ ID NO: 1266 - RNA, 97 nt, Medicago
ugauguucuacagggucgacaugagagcacaugaagcuaucaugguugucuauguuauccaacucauguguucucaggucgccccugcugaauuuuc

SEQ ID NO: 1267 - RNA, 109 nt, Nicotiana
aaguguucaacaggggcaaccugagaucacauauugucauuuuucuuuaguuguugagucugguucaauaucucacuauguguucucaggucgccccugucgaauuauu

SEQ ID NO: 1268 - RNA, 86 nt, Oryza
gaguuccuacaggggcgagcugggaacacacggugaugaggcggucuggucuuucguguguucucaggucgccccugccgggacuc

SEQ ID NO: 1269 - RNA, 126 nt, Zea
caguuccggcgggggcggacugggaacacaugggaaugagaugagaucauugcucggucgugcuggccugggccgucggcgcgcguugaucuugcauguguucucaggucgcccccggagggccuu

SEQ ID NO: 1270 - RNA, 94 nt, Medicago
aaaucagcuauagggcuucucuuucuuggcaggaaauuaucaugaccauuccaucaugugucuugccaaaggagaguugcccuguugcuguuuu

SEQ ID NO: 1271 - RNA, 102 nt, Populus
uaaagaauaacagggcuuuauccuccuuuggcaaacagaacauggaaauaaaugccugcauauuucuguuuugccaagggagaauugcccugccauucgauu

SEQ ID NO: 1272 - RNA, 21 nt, Arabidpsis thaliana
uccaaagggaucgcauugauc

SEQ ID NO: 1273 - RNA, 66 nt, Arabidpsis thaliana
  misc_feature (8)..(33): n is a, c, g, or u
cuaauuannnnnnnnnnnnnnnnnnnnnnnnnnggcaaauaaaucacaaaaauuugcuugguuuug

SEQ ID NO: 1274 - RNA, 97 nt, Arabidpsis thaliana
  misc_feature (9)..(26): n is a, c, g, or u
aauuaagcnnnnnnnnnnnnnnnnnnuuguuuuucuuuuccuucucaaucgaaagauggaagaaaaacaannnnnnnnnnnnnngcuuacuuuuccg

SEQ ID NO: 1275 - RNA, 21 nt, Oryza sativa
uccaaagggaucgcauugauc

SEQ ID NO: 1276 - RNA, 50 nt, Oryza sativa
  misc_feature (34)..(42): n is a, c, g, or u
cuucaucgcucucgcucgcuuccauggcggucgnnnnnnnnnagcagcuu

SEQ ID NO: 1277 - RNA, 20 nt, Arabidopsis thaliana
uuggcauucuguccaccucc

SEQ ID NO: 1278 - RNA, 54 nt, Arabidopsis thaliana
  misc_feature (30)..(52): n is a, c, g, or u
ucucucuauauuuauguguaauaaguguannnnnnnnnnnnnnnnnnnnnnnga

SEQ ID NO: 1279 - RNA, 44 nt, Arabidopsis thaliana
uauacauauaugcauguguauauauauaugcgucuugugugaaa

SEQ ID NO: 1280 - RNA, 20 nt, Oryza sativa
uuggcauucuguccaccucc

SEQ ID NO: 1281 - RNA, 43 nt, Oryza sativa
uugucgaauccucagagacagaaaucucauaccuguugaucuu

SEQ ID NO: 1282 - RNA, 41 nt, Arabidopsis thaliana
acaaagcuggagacaaugcgaucccuuuggaugucuucuug

SEQ ID NO: 1283 - RNA, 21 nt, Arabidopsis thaliana
uccaaagggaucgcauugauc

SEQ ID NO: 1284 - RNA, 41 nt, Arabidopsis thaliana
gguagguacgaaacaaugcgaucccuuuggaugucgucuug

SEQ ID NO: 1285 - RNA, 21 nt, Arabidopsis thaliana
uccaaagggaucgcauugauc

SEQ ID NO: 1286 - RNA, 41 nt, Arabidopsis thaliana
agcaaguaugaaacaaugcgaucccuuuggaugucuucaug

SEQ ID NO: 1287 - RNA, 21 nt, Arabidopsis thaliana
uccaaagggaucgcauugauc

SEQ ID NO: 1288 - RNA, 41 nt, Arabidopsis thaliana
gccaagcuagagaccaugcgaucccuuuggaugucaucuug

SEQ ID NO: 1289 - RNA, 21 nt, Arabidopsis thaliana
uccaaagggaucgcauugauc

SEQ ID NO: 1290 - RNA, 41 nt, Arabidopsis thaliana
cuuaccuuugggucagagcgaucccuuuggcaauggcaaug

SEQ ID NO: 1291 - RNA, 21 nt, Arabidopsis thaliana
uccaaagggaucgcauugauc

SEQ ID NO: 1292 - RNA, 41 nt, Arabidopsis thaliana
cuguuguggaaggagguugacagaaugccaaacauaugguc

SEQ ID NO: 1293 - RNA, 21 nt, Arabidopsis thaliana
uuuggcauucuguccaccucc

SEQ ID NO: 1294 - RNA, 41 nt, Arabidopsis thaliana
gagacagucagaguuccuccaaacacuucauuuuaacucgu

SEQ ID NO: 1295 - RNA, 21 nt, Arabidopsis thaliana
cugaaguguuugggggaacuc

SEQ ID NO: 1296 - RNA, 41 nt, Arabidopsis thaliana
gaggccgccaucguucaagaaagccuguggaaggccaaaau

SEQ ID NO: 1297 - RNA, 21 nt, Arabidopsis thaliana
uuccacagcuuucuugaacug

SEQ ID NO: 1298 - RNA, 41 nt, Arabidopsis thaliana
gaggccgucaucguucaagaaagccuguggaaguccaaucu

SEQ ID NO: 1299 - RNA, 21 nt, Arabidopsis thaliana
uuccacagcuuucuugaacug

SEQ ID NO: 1300 - RNA, 41 nt, Arabidopsis thaliana
guggccgcaaccguucaagaaagccuguggaaacuccaacc

SEQ ID NO: 1301 - RNA, 21 nt, Arabidopsis thaliana
uuccacagcuuucuugaacug

SEQ ID NO: 1302 - RNA, 41 nt, Arabidopsis thaliana
gaggucguccucguucaagaaagcauguggaaccuccuuau

SEQ ID NO: 1303 - RNA, 21 nt, Arabidopsis thaliana
uuccacagcuuucuugaacug

SEQ ID NO: 1304 - RNA, 41 nt, Arabidopsis thaliana
agagccguccucguucaagaaagcauguggaaucaucucac

SEQ ID NO: 1305 - RNA, 21 nt, Arabidopsis thaliana
uuccacagcuuucuugaacug

SEQ ID NO: 1306 - RNA, 41 nt, Arabidopsis thaliana
gaggucguaaacguucaagaaagcuuguggaaucuucuucu

SEQ ID NO: 1307 - RNA, 21 nt, Arabidopsis thaliana
uuccacagcuuucuugaacug

SEQ ID NO: 1308 - RNA, 41 nt, Arabidopsis thaliana
uacuacgauuaaucaaugcugcacucaaugacgaacucuuc

SEQ ID NO: 1309 - RNA, 21 nt, Arabidopsis thaliana
ucauugagugcagcguugaug

SEQ ID NO: 1310 - RNA, 41 nt, Arabidopsis thaliana
ugcuacgacuagucaacgcugcacuuaaugaagaacucuuu

SEQ ID NO: 1311 - RNA, 21 nt, Arabidopsis thaliana
ucauugagugcagcguugaug

SEQ ID NO: 1312 - RNA, 41 nt, Arabidopsis thaliana
uucucaggcuaaucaaugcugcacuuaaugacgagcucuuu

SEQ ID NO: 1313 - RNA, 21 nt, Arabidopsis thaliana
ucauugagugcagcguugaug

SEQ ID NO: 1314 - RNA, 41 nt, Arabidopsis thaliana
auucuuuccaaagggguuuccugagaucacaaaggccaagu

SEQ ID NO: 1315 - RNA, 21 nt, Arabidopsis thaliana
uguguucucaggucaccccuu

SEQ ID NO: 1316 - RNA, 41 nt, Arabidopsis thaliana
agugccgucaugcgggugaccugggaaacauaaaugccaau

SEQ ID NO: 1317 - RNA, 21 nt, Arabidopsis thaliana
uguguucucaggucaccccuu

SEQ ID NO: 1318 - RNA, 41 nt, Arabidopsis thaliana
cuaauccuucaaggugugaccugagaaucacaacacaaaac

SEQ ID NO: 1319 - RNA, 21 nt, Arabidopsis thaliana
uguguucucaggucaccccuu